(19) United States
(12) Patent Application Publication
(10) Pub. No.: US 2003/0232421 A1
(43) Pub. Date: Dec. 18, 2003

(54) Protein-protein interactions in adipocyte cells (3)

(75) Inventors: Pierre Legrain, Paris (FR); Simon Whiteside, Cambridge (GB); Jen-I Mao, Palo Alto, CA (US); Irina Khrebtukova, San Francisco, CA (US); Shujun Luo, Berkeley, CA (US)

Correspondence Address: LERNER, DAVID, LITTENBERG,, KRUMHOLZ & MENTLIK, 600 SOUTH AVENUE WEST, WESTFIELD, NJ, 07090, US

(73) Assignee: Hybrigenics, Paris (FR)

(21) Appl. No.: 10/139,794

(22) Filed: May 6, 2002

Related U.S. Application Data
(60) Provisional application No. 60/288,885, filed on May 4, 2001.

(51) Int. Cl. A61K 31/00; G01N 33/53; C07H 21/04; C12N 9/64; C12P 21/02; C12N 5/06

(52) U.S. Cl. 435/226; 435/69.1; 435/7.1; 435/320.1; 435/325; 536/23.2; 514/1

(57) ABSTRACT

The present invention relates to protein-protein interactions of adipocytes, e.g., complexes of such polypeptides, polynucleotides encoding the polypeptides and complexes thereof, fragments of the polypeptides, antibodies to the complexes, Selected Interacting Domains (SID) which are identified due to the protein-protein interactions, methods for screening drugs for agents which modulate the interaction of proteins and pharmaceutical compositions that modulate the protein-protein interactions.

### PRIORITY

This application claims priority on the basis of U.S. Provisional Application No. 60/288,885 filed May 4, 2001, incorporated herein by reference.

### BACKGROUND

Most biological processes involve specific protein-protein interactions. Protein-protein interactions enable two or more proteins to associate. A large number of non-covalent bonds form between the proteins when two protein surfaces are precisely matched. These bonds account for the specificity of recognition. Thus, protein-protein interactions are involved, for example, in the assembly of enzyme subunits, in antibody-antigen recognition, in the formation of biochemical complexes, in the correct folding of proteins, in the metabolism of proteins, in the transport of proteins, in the localization of proteins, in protein turnover, in first translation modifications, in the core structures of viruses and in signal transduction.

General methodologies to identify interacting proteins or to study these interactions have been developed. Among these methods are the two-hybrid system originally developed by Fields and co-workers and described, for example, in U.S. Pat. Nos. 5,283,173, 5,468,614 and 5,667,973, which are hereby incorporated by reference.

The earliest and simplest two-hybrid system, which acted as basis for development of other versions, is an in vivo assay between two specifically constructed proteins. The first protein, known in the art as the “bait protein” is a chimeric protein which binds to a site on DNA upstream of a reporter gene by means of a DNA-binding domain or BD. Commonly, the binding domain is the DNA-binding domain from either Gal4 or native E. coli LexA and the sites placed upstream of the reporter are Gal4 binding sites or LexA operators, respectively.

The second protein is also a chimeric protein known as the “prey” in the art. This second chimeric protein carries an activation domain or AD. This activation domain is typically derived from Gal4, from VP16 or from B42.

Besides the-two hybrid systems, other improved systems have been developed to detect protein-protein interactions. For example, a two-hybrid plus one system was developed that allows the use of two proteins as bait to screen available cDNA libraries to detect a third partner. This method permits the detection between proteins that are part of a larger protein complex such as the RNA polymerase II holoenzyme and the TFIIH or TFIID complexes. Therefore, this method, in general, permits the detection of ternary complex formation as well as inhibitors preventing the interaction between the two previously defined fused proteins.

Another advantage of the two-hybrid plus one system is that it allows or prevents the formation of the transcriptional activator since the third partner can be expressed from a conditional promoter such as the methionine-repressed Met25 promoter which is positively regulated in medium lacking methionine. The presence of the methionine-regulated promoter provides an excellent control to evaluate the activation or inhibition properties of the third partner due to its “on” and “off” switch for the formation of the transcriptional activator. The three-hybrid method is described, for example in Tirode et al., The Journal of Biological Chemistry, 272, No. 37 pp. 22995-22999 (1997) incorporated herein by reference.

Besides the two and two-hybrid plus one systems, yet another variant is that described in Vidal et al, Proc. Natl. Sci. 93 pgs. 10315-10320 called the reverse two- and one-hybrid systems where a collection of molecules can be screened that inhibit a specific protein-protein or protein-DNA interactions, respectively.

A summary of the available methodologies for detecting protein-protein interactions is described in Vidal and Legrain (Nucleic Acids Research Vol. 27, No. 4 pgs. 919-929 (1999)) and Legrain and Selig (FEBS Letters 480 pgs. 32-36 (2000)) which references are incorporated herein by reference.

However, the above conventionally used approaches and especially the commonly used two-hybrid methods have their drawbacks. For example, it is known in the art that, more often than not, false positives and false negatives exist in the screening method. In fact, a doctrine has been developed in this field for interpreting the results and in common practice an additional technique such as co-immunoprecipitation or gradient sedimentation of the putative interactors from the appropriate cell or tissue type are generally performed. The methods used for interpreting the results are described by Brent and Finley (Jr. in Ann. Rev. Genet., 31 pgs. 663-704 (1997)). Thus, the data interpretation is very questionable using the conventional systems.

One method to overcome the difficulties encountered with the methods in the prior art is described in WO99/42612, incorporated herein by reference. This method is similar to the two-hybrid system described in the prior art in that it also uses bait and prey polypeptides. However, the difference with this method is that a step of mating at least one first haploid recombinant yeast cell containing the prey polypeptide to be assayed with a second haploid recombinant yeast cell containing the bait polynucleotide is performed. Of course the person skilled in the art would appreciate that either the first recombinant yeast cell or the second recombinant yeast cell also contains at least one detectable reporter gene that is activated by a polypeptide including a transcriptional activation domain.

The method described in WO99/42612 permits the screening of more prey polynucleotides with a given bait polynucleotide in a single step than in the prior art systems due to the cell to cell mating strategy between haploid yeast cells. Furthermore, this method is more thorough and reproducible, as well as sensitive. Thus, the presence of false negatives and/or false positives is extremely minimal as compared to the conventional prior art methods.

The causes of Non-insulin dependent diabetes mellitus (NIDDM) and obesity are often related to defects or problems with adipose tissue. Adipocytes play a critical role in lipid storage and metabolism. Adipocytes also act as endocrine cells to influence physiological parameters such as insulin sensitivity and body weight (Flier, et al., Cell, (1995) 80: 15-18). For example, the ob gene encodes leptin, an adipocyte secreted endocrine factor (Zhang, et al., Nature (1994) 372: 425-432). Leptin has been shown to reduce body weight and blood glucose in obese, diabetic rodents (Pelleymounter, et al., Science, (1995) 269: 540-543).

NIDDM is treated predominately with insulin. However, insulin is not convenient to use in that it must be injected 2-4 times per day and must be stored properly to prevent loss of efficacy. Other drugs used to treat NIDDM include troglitazone (“Rezulin”), a PPARY agonist, Glucophage and sulfonylureas. Unfortunately, there are safety concerns related to the use of these drugs. The identification of safe, effective, orally available drugs for the treatment of NIDDM would greatly enhance the quality of life of patients who suffer from this disease.

Several adipocyte-specific enzymes and receptors have been shown to be important targets for anti-obesity and anti-diabetic drug discovery. For example, agonists of the β3 adrenergic receptor, which is found predominantly in the adipose tissue in man (Arner, et al., New England Journal of Medicine, (1995) 333: 382-383), have anti-obesity and anti-diabetic properties in rodents and are currently in phase 11/111 trials in man. The thiazolidinedione class of compounds (TZDs), including troglitazone and ciglitazone, has been shown to improve insulin sensitivity and thereby reduce hyperglycemia and hyperlipidemia conditions in rodents and in humans (Saltiel, et al., Diabetes, (1996) 45: 1661-1669; Sreenan, et al., American Journal Physiol, (1996) 271: E742-E747; Nolan, et al., New England Journal of Medicine, (1994) 331: 1188-1193. Troglitazone (“Rezulin”) is approved for use in the U.S. and Japan. Many TZDs, including troglitazone and ciglitazone, are potent activators of Peroxisome Proliferator Activated Receptor gamma (PPARy), a member of the nuclear receptor family of transcription factors (Tontonoz, et al., Cell, (1994) 79: 1147-1156; Lehmann, et al., Journal of Biological Chemistry, (1995) 270: 12953-12955). PPARB, is a key regulator of adipocyte differentiation and is most abundant in adipose tissue.

This shows that it is still needed to explore all mechanisms of adipocyte differentiation and to identify drug targets for metabolism diseases.

The present invention has allowed the identification of protein interactions of the Wnt pathway that is a pathway of particular interest: combined work in flies, worms and mammals has produced the main outline of the canonical Wnt pathway that play key roles during normal animal development. Wnts are a family of autocrine and paracrine factors that regulate cell growth and development.

In unstimulated cells, free cytoplasmic beta-catenin (βCat) protein is phosphorylated by a multiprotein complex containing Axin (or its homologue Conductin), Glycogen Synthase Kinase 3β (GSK3β) and the tumour suppressor protein Adenomatous polyposis coli (APC). Interaction between Axin and GSK3β in the complex facilitates efficient phosphorylation of βCat, most likely at critical Serine and Threonine residues in its N-terminus. This phosphorylation event earmarks βCat for interaction with and ubiquitination by the SCF complex (containing the F-box protein β-TrCP) and subsequent degradation by the 26S proteasome.

Signalling is initiated when Wnt ligands bind to the transmembrane receptors of the Frizzled (Fz) family. This in turn leads to the recruitement to the plasma membrane of the members of the Dishevelled (DvI) family of cytoplasmic proteins, of which three, DvI1, 2 and 3, have been identified in mammals. Dishevelled appears to inhibit the Axin/APC/GSK3β complex by direct binding to Axin, although the precise molecular mechanism is unknown. Whether DvI proteins bind directly to Fz receptors or whether intermediary proteins are involved in the signal transmission event is, as yet, unknown.

Thus, once hypophosphorylated as a result of Wnt signalling, βCat is stabilised, and translocates to the nucleus where it binds to the TCF/LEF family of transcription factors to regulate the expression of Wnt target genes (see FIG. 1).

In addition to an important role in colorectal cancer, the Wnt signalling pathway has recently been demonstrated to play a crucial role in the process of adipogenesis (Ross et al., Science (2000), 289, 950ff). Wnt signalling maintains pre-adipocytes in an undifferentiated state through inhibition of the adipogenic transcription factors CCAAT/enhancer binding protein alpha (C/EBPα) and peroxisome proliferator-activated receptor gamma (PPARα). Disruption of Wnt signalling in pre-adipocytes or myoblasts can cause these cells to differentiate into mature adipocytes.

The non ATPase subunit of the 26S proteasome, PSMD8, is the human orthologue of the Nin1 protein of S. cerevisiae, a protein implicated in the regulation of cell cycle and the G1/S and G2/M transitions (Kominami et a. (1995) EMBO J. 14, 3105ff).

### SUMMARY OF THE INVENTION

Thus, the present invention relates to identifying protein-protein interactions in adipocytes. For this purpose, the research on proteins selected after comparison of protein expression profiles in undifferentiated and in differentiated adipocytes was undertaken. Proteins were selected that are specifically over-expressed in differentiated adipocytes.

The present invention also relates to identifying specific protein-protein interactions in differentiated or undifferentiated adipocytes for the development of more effective and better targeted therapeutic applications.

The present invention also identifies complexes of polypeptides or polynucleotides encoding the polypeptides and fragments of the polypeptides of adipocytes.

Thus the present invention relates to a complex of interacting proteins of columns 1 and 4 of Table 2.

The present invention also identifies antibodies to these complexes of polypeptides or polynucleotides encoding the polypeptides and fragments of the polypeptides of adipocytes including polyclonal, as well as monoclonal antibodies that are used for detection.

The present invention also discloses selected interacting domains of the polypeptides, called SID polypeptides.

The present invention also discloses selected interacting domains of the polynucleotides, called SID polynucleotides.

The present invention relates to the generation of protein-protein interactions maps called PIMs.

The present invention also discloses a method for screening drugs for agents which modulate the interaction of proteins and pharmaceutical compositions that modulate the protein-protein interactions in adipocytes.

In another aspect, the present invention relates to administering the nucleic acids of the present invention via gene therapy.

In yet another aspect the present invention provides protein chips or protein microarrays.

In yet another aspect the present invention provides a report in, for example paper, electronic and/or digital form, concerning the protein-protein interactions, the modulating compounds and the like as well as a PIM.

### BRIEF DESCRIPTION OF THE DRAWINGS

FIG. 1 is a schematic representation of the Wnt pathway: left, in the absence of Wnt stimulation; right, in the presence of Wnt stimulation. (Protein names refer to abbreviations used in the background text).

FIG. 2 is a schematic representation of the pB27 plasmid.

FIG. 3 is a schematic representation of the pB20 plasmid.

FIG. 4 is a schematic representation of the pP6 plasmid.

FIG. 5 is a schematic representation of vectors expressing the T25 fragment.

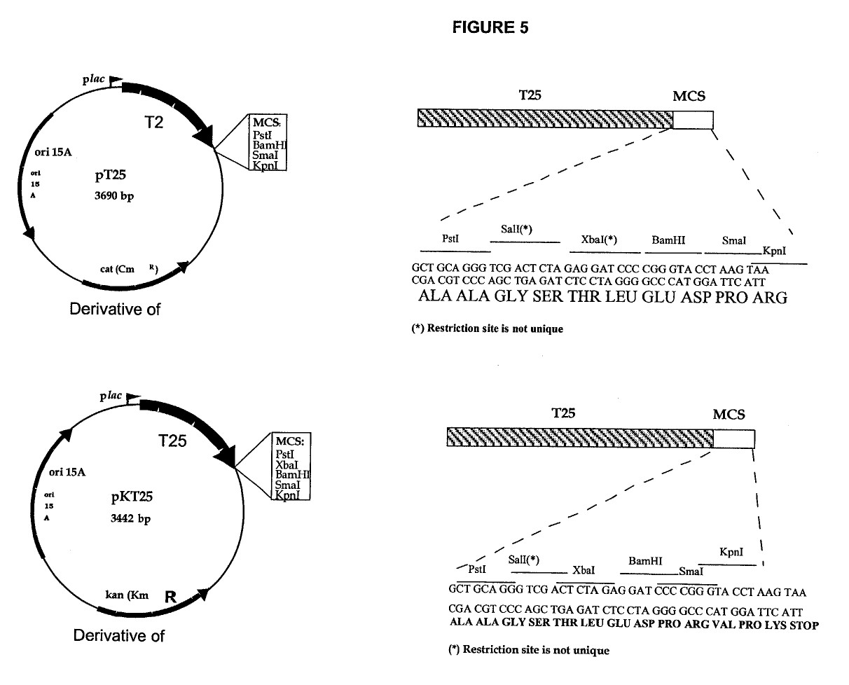

FIG. 6 is a schematic representation of vectors expressing the T18 fragment.

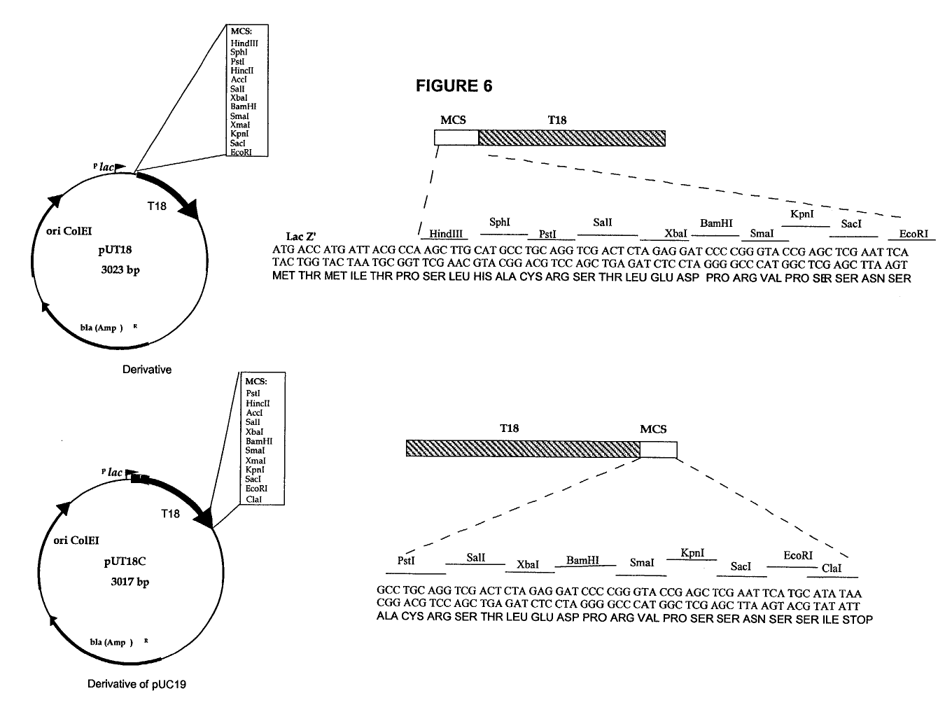

FIG. 7 is a schematic representation of various vectors of pCmAHL1, pT25 and pT18.

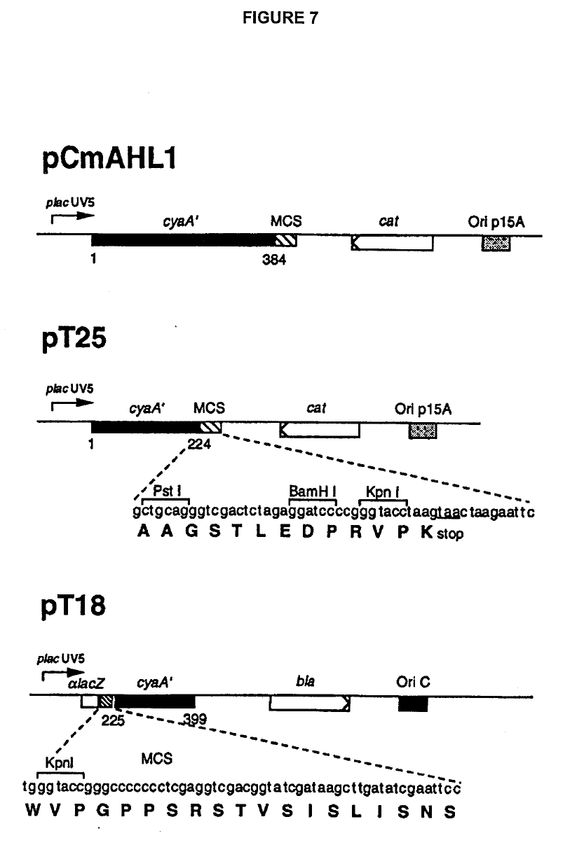

FIG. 8 is a schematic representation identifying the SID's of adipocytes. In this figure the “Full-length prey protein” is the Open Reading Frame (ORF) or coding sequence (CDS) where the identified prey polypeptides are included. The Selected Interaction Domain (SID®) is determined by the commonly shared polypeptide domain of every selected prey fragment.

FIG. 9 is a protein map (PIM).

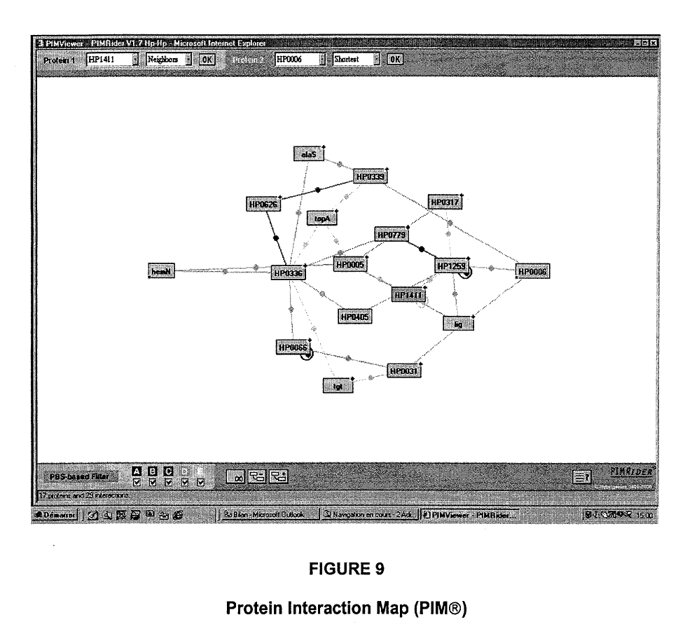

FIG. 10 is a schematic representation of the pB28 plasmid.

### DETAILED DESCRIPTION OF THE INVENTION

As used herein the terms “polynucleotides”, “nucleic acids” and “oligonucleotides” are used interchangeably and include, but are not limited to RNA, DNA, RNA/DNA sequences of more than one nucleotide in either single chain or duplex form. The polynucleotide sequences of the present invention may be prepared from any known method including, but not limited to, any synthetic method, any recombinant method, any ex vivo generation method and the like, as well as combinations thereof.

Polynucleotides which can hybridize to any of the polynucleotides discussed above are also covered by the present invention. Such polynucleotides are referred to herein as “hybridizing” polynucleotides. Hybridizing polynucleotides can be useful as probes or primers, for example.

According to an embodiment of the present invention, such hybridizing molecules are at least about 10 (e.g., 10) nucleotides in length. In another embodiment, they are at least about 25 (e.g., at least 25) or at least about 50 (e.g., at least 50) nucleotides in length.

In yet another embodiment, the hybridizing molecules will hybridize to such molecules under stringent hybridization conditions. One example of stringent hybridization conditions is where attempted hybridization is carried out at a temperature of from about 35° C. to about 65° C. using a salt solution which is about 0.9 molar. However, the skilled person will be able to vary such conditions as appropriate in order to take into account variables such as probe length, base composition, type of ions present, etc.

The term “polypeptide” means herein a polymer of amino acids having no specific length. Thus, peptides, oligopeptides and proteins are included in the definition of “polypeptide” and these terms are used interchangeably throughout the specification, as well as in the claims. The term “polypeptide” does not exclude post-translational modifications such as polypeptides having covalent attachment of glycosyl groups, aceteyl groups, phosphate groups, lipid groups and the like. Also encompassed by this definition of “polypeptide” are homologs thereof.

By the term “homologs” is meant structurally similar genes contained within a given species, orthologs are functionally equivalent genes from a given species or strain, as determined for example, in a standard complementation assay. Thus, a polypeptide of interest can be used not only as a model for identifying similiar genes in given strains, but also to identify homologs and orthologs of the polypeptide of interest in other species. The orthologs, for example, can also be identified in a conventional complementation assay. In addition or alternatively, such orthologs can be expected to exist in bacteria (or other kind of cells) in the same branch of the phylogenic tree, as set forth, for example, at ftp://ftp.cme.msu.edu/pub/rdp/SSU-rRNA/SSU/Prok.phylo.

As used herein the term “prey polynucleotide” means a chimeric polynucleotide encoding a polypeptide comprising (i) a specific domain; and (ii) a polypeptide that is to be tested for interaction with a bait polypeptide. The specific domain is preferably a transcriptional activating domain.

As used herein, a “bait polynucleotide” is a chimeric polynucleotide encoding a chimeric polypeptide comprising (i) a complementary domain; and (ii) a polypeptide that is to be tested for interaction with at least one prey polypeptide. The complementary domain is preferably a DNA-binding domain that recognizes a binding site that is further detected and is contained in the host organism.

As used herein “complementary domain” is meant a functional constitution of the activity when bait and prey are interacting; for example, enzymatic activity.

As used herein “specific domain” is meant a functional interacting activation domain that may work through different mechanisms by interacting directly or indirectly through intermediary proteins with RNA polymerase 11 or III-associated proteins in the vicinity of the transcription start site.

As used herein the term “complementary” means that, for example, each base of a first polynucleotide is paired with the complementary base of a second polynucleotide whose orientation is reversed. The complementary bases are A and T (or A and U) or C and G.

The term “sequence identity” refers to the identity between two peptides or between two nucleic acids. Identity between sequences can be determined by comparing a position in each of the sequences which may be aligned for the purposes of comparison. When a position in the compared sequences is occupied by the same base or amino acid, then the sequences are identical at that position. A degree of sequence identity between nucleic acid sequences is a function of the number of identical nucleotides at positions shared by these sequences. A degree of identity between amino acid sequences is a function of the number of identical amino acid sequences that are shared between these sequences.

Examples of comparison methods are the following: optimal alignment of sequences for determining a comparison window may be conducted by the local homology algorithm of Smith and Waterman (J. Theor. Biol., 91 (2) pgs. 370-380 (1981), by the homology alignment algorithm of Needleman and Wunsch, J. Miol. Biol., 48(3) pgs. 443-453 (1972), by the search for similarity via the method of Pearson and Lipman, PNAS, USA, 85(5) pgs. 2444-2448 (1988), by computerized implementations of these algorithms (GAP, BESTFIT, FASTA and TFASTA in the Wisconsin Genetics Software Package Release 7.0, Genetic Computer Group, 575, Science Drive, Madison, Wis.) or by inspection.

The term “sequence identity” means that two polynucleotide sequences are identical (i.e., on a nucleotide by nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size) and multiplying the result by 100 to yield the percentage of sequence identity. The same process can be applied to polypeptide sequences.

The percentage of sequence identity of a nucleic acid sequence or an amino acid sequence can also be calculated using BLAST software (Version 2.06 of September 1998) with the default or user defined parameter.

The term “sequence similarity” means that amino acids can be modified while retaining the same function. It is known that amino acids are classified according to the nature of their side groups and some amino acids such as the basic amino acids can be interchanged for one another while their basic function is maintained.

The term “isolated” as used herein means that a biological material such as a nucleic acid or protein has been removed from its original environment in which it is naturally present. For example, a polynucleotide present in a plant, mammal or animal is present in its natural state and is not considered to be isolated. The same polynucleotide separated from the adjacent nucleic acid sequences in which it is naturally inserted in the genome of the plant or animal is considered as being “isolated.”

The term “isolated” is not meant to exclude artificial or synthetic mixtures with other compounds, or the presence of impurities which do not interfere with the biological activity and which may be present, for example, due to incomplete purification, addition of stabilizers or mixtures with pharmaceutically acceptable excipients and the like.

“Isolated polypeptide” or “isolated protein” as used herein means a polypeptide or protein which is substantially free of those compounds that are normally associated with the polypeptide or protein in a naturally state such as other proteins or polypeptides, nucleic acids, carbohydrates, lipids and the like.

The term “purified” as used herein means at least one order of magnitude of purification is achieved, preferably two or three orders of magnitude, most preferably four or five orders of magnitude of purification of the starting material or of the natural material. Thus, the term “purified” as utilized herein does not necessarily mean that the material is 100% purified so as to exclude any other material.

The term “variants” when referring to, for example, polynucleotides encoding a polypeptide variant of a given reference polypeptide are polynucleotides that differ from the reference polypeptide but generally maintain their functional characteristics of the reference polypeptide. A variant of a polynucleotide may be a naturally occurring allelic variant or it may be a variant that is known naturally not to occur. Such non-naturally occurring variants of the reference polynucleotide can be made by, for example, mutagenesis techniques, including those mutagenesis techniques that are applied to polynucleotides, cells or organisms.

Generally, differences are limited so that the nucleotide sequences of the reference and variant are closely similar overall and, in many regions identical.

Variants of polynucleotides according to the present invention include, but are not limited to, nucleotide sequences which are at least about 95% (e.g., 95%) identical after alignment to the reference polynucleotide encoding the reference polypeptide. These variants can also have 96%, 97%, 98% and 99.99% sequence identity to the reference polynucleotide.

Nucleotide changes present in a variant polynucleotide may be silent, which means that these changes do not alter the amino acid sequences encoded by the reference polynucleotide.

Substitutions, additions and/or deletions can involve one or more nucleic acids. Alterations can produce conservative or non-conservative amino acid substitutions, deletions and/or additions.

Variants of a prey or a SID polypeptide encoded by a variant polynucleotide can possess a higher affinity of binding and/or a higher specificity of binding to its protein or polypeptide counterpart, against which it has been initially selected. In another context, variants can also loose their ability to bind to their protein or polypeptide counterpart.

By “fragment of a polynucleotide” or “fragment of a SID® polynucleotide” is meant that fragments of these sequences have at least about 12 (e.g., at least 12) consecutive nucleotides or between about 12 (e.g., 12) and about 5,000 consecutive nucleotides, or between about 12 and about 10,000 consecutive nucleotides, or between about 12 and about 20,000 consecutive nucleotides.

By “fragment of a polypeptide” or fragment of a SID® polypeptide is meant that fragments of these sequences have at least about 4 (e.g., at least 4) consecutive amino acids, or between about 4 (e.g., 4) and about 1,700 consecutive amino acids, or between about 4 and about 3,300 consecutive amino acids, or between about 4 and about 6,600 consecutive amino acids.

As used herein, “drug metabolism” is meant the study of how drugs are processed and broken down by the body. Drug metabolism can involve the study of enzymes that break down drugs, the study of how different drugs interact within the body and how diet and other ingested compounds affect the way the body processes drugs.

As used herein, “metabolism” means the sum of all of the enzyme-catalyzed reactions in living cells that transform organic molecules.

By “secondary metabolism” is meant pathways producing specialized metabolic products that are not found in every cell.

As used herein, “SID” means a Selected Interacting Domain and is identified as follows: for each bait polypeptide screened, selected prey polypeptides are compared. Overlapping fragments in the same ORF or CDS define the selected interacting domain.

As used herein the term “PIM” means a protein-protein interaction map. This map is obtained from data acquired from a number of separate screens using different bait polypeptides and is designed to map out all of the interactions between the polypeptides.

The term “affinity of binding”, as used herein, can be defined as the affinity constant Ka when a given SID polypeptide of the present invention which binds to a polypeptide and is the following mathematical relationship:

$$Ka = \frac{[ SID / polypeptide complex ]}{[ free SID ] [ free polypeptide ]}$$

wherein [free SID], [free polypeptide] and [SID/polypeptide complex] consist of the concentrations at equilibrium respectively of the free SID polypeptide, of the free polypeptide onto which the SID polypeptide binds and of the complex formed between SID polypeptide and the polypeptide onto which said SID polypeptide specifically binds.

The affinity of a SID polypeptide of the present invention or a variant thereof for its polypeptide counterpart can be assessed, for example, on a Biacore™ apparatus marketed by Amersham Pharmacia Biotech Company such as described by Szabo et al. (Curr. Opin. Struct. Biol. 5 pgs. 699-705 (1995)) and by Edwards and Leartherbarrow (Anal. Biochem 246 pgs. 1-6 (1997)).

As used herein the phrase “at least the same affinity” with respect to the binding affinity between a SID polypeptide of the present invention to another polypeptide means that the Ka is identical or can be at least two-fold, at least three-fold or at least five fold greater than the Ka value of reference.

As used herein, the term “modulating compound” means a compound that inhibits or stimulates or can act on another protein which can inhibit or stimulate the protein-protein interaction of a complex of two polypeptides or the protein-protein interaction of two polypeptides.

More specifically, the present invention comprises complexes of polypeptides or polynucleotides encoding the polypeptides composed of a bait polypeptide, or a bait polynucleotide encoding a bait polypeptide and a prey polypeptide or a prey polynucleotide encoding a prey polypeptide. The prey polypeptide or prey polynucleotide encoding the prey polypeptide is capable of interacting with a bait polypeptide of interest in various hybrid systems.

As described in the Background of the present invention, there are various methods known in the art to identify prey polypeptides that interact with bait polypeptides of interest. These methods include, but are not limited to, generic two-hybrid systems as described by Fields et al. (Nature, 340:245-246 (1989)) and more specifically in U.S. Pat. Nos. 5,283,173, 5,468,614 and 5,667,973, which are hereby incorporated by reference; the reverse two-hybrid system described by Vidal et al. (supra); the two plus one hybrid method described, for example, in Tirode et al. (supra); the yeast forward and reverse ‘n’-hybrid systems as described in Vidal and Legrain (supra); the method described in WO 99/42612; those methods described in Legrain et al. (FEBS Letters 480 pgs. 32-36 (2000)) and the like.

The present invention is not limited to the type of method utilized to detect protein-protein interactions and therefore any method known in the art and variants thereof can be used. It is however better to use the method described in WO99/42612 or WO00/66722, both references incorporated herein by reference due to the methods' sensitivity, reproducibility and reliability.

Protein-protein interactions can also be detected using complementation assays such as those described by Pelletier et al. at http://www.abrf.org/JBT/Articles/JBT0012/jbt0012.html, WO00/07038 and WO98/34120.

Although the above methods are described for applications in the yeast system, the present invention is not limited to detecting protein-protein interactions using yeast, but also includes similar methods that can be used in detecting protein-protein interactions in, for example, mammalian systems as described, for example in Takacs et al. (Proc. Natl. Acad. Sci., USA, 90 (21):10375-79 (1993)) and Vasavada et al. (Proc. Natl. Acad. Sci., USA, 88 (23):10686-90 (1991)), as well as a bacterial two-hybrid system as described in Karimova et al. (1998), WO99/28746, WO00/66722 and Legrain et al. (FEBS Letters, 480 pgs. 32-36 (2000)).

The above-described methods are limited to the use of yeast, mammalian cells and Escherichia coli cells, the present invention is not limited in this manner. Consequently, mammalian and typically human cells, as well as bacterial, yeast, fungus, insect, nematode and plant cells are encompassed by the present invention and may be transfected by the nucleic acid or recombinant vector as defined herein.

Examples of suitable cells include, but are not limited to, VERO cells, HELA cells such as ATCC No. CCL2, CHO cell lines such as ATCC No. CCL61, COS cells such as COS-7 cells and ATCC No. CRL 1650 cells, W138, BHK, HepG2, 3T3 such as ATCC No. CRL6361, A549, PC12, K562 cells, 293 cells, Sf9 cells such as ATCC No. CRL1711 and Cv1 cells such as ATCC No. CCL70.

Other suitable cells that can be used in the present invention include, but are not limited to, prokaryotic host cells strains such as Escherichia coli, (e.g., strain DH5-α), Bacillus subtilis, Salmonella typhimurium, or strains of the genera of Pseudomonas, Streptomyces and Staphylococcus.

Further suitable cells that can be used in the present invention include yeast cells such as those of Saccharomyces such as Saccharomyces cerevisiae.

The bait polynucleotide, as well as the prey polynucleotide can be prepared according to the methods known in the art such as those described above in the publications and patents reciting the known method per se.

The bait and the prey polynucleotides of the present invention are obtained from adipocytes' cDNA (either from human differentiated PAZ6 adipocytes or from human undifferentiated PAZ6 adipocytes), or variants of cDNA fragment from a library of human differentiated PAZ6 adipocytes or of human undifferentiated PAZ6 adipocytes, and fragments from the genome or transcriptome of human differentiated PAZ6 adipocytes or of human undifferentiated PAZ6 adipocytes ranging from about 12 to about 5,000, or about 12 to about 10,000 or from about 12 to about 20,000. The prey polynucleotide is then selected, sequenced and identified.

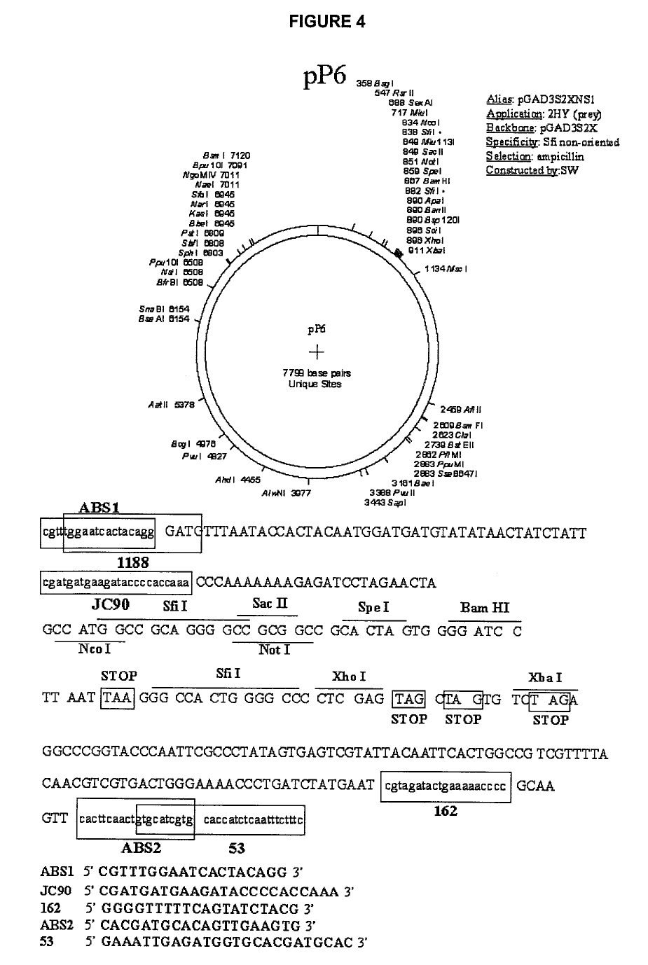

Human differentiated PAZ6 adipocytes and human undifferentiated PAZ6 adipocytes prey libraries are prepared from the human differentiated PAZ6 adipocytes and human undifferentiated PAZ6 adipocytes, respectively, and constructed in the specially designed prey vector pP6 as shown in FIG. 4 after ligation of suitable linkers such that every cDNA insert is fused to a nucleotide sequence in the vector that encodes the transcription activation domain of a reporter gene. Any transcription activation domain can be used in the present invention. Examples include, but are not limited to, Gal4,YP16, B42, His and the like. Toxic reporter genes, such as CAT^{R}, CYH2, CYH1, URA3, bacterial and fungi toxins and the like can be used in reverse two-hybrid systems.

The polypeptides encoded by the nucleotide inserts of the human differentiated PAZ6 adipocytes or human undifferentiated PAZ6 adipocytes prey library thus prepared are termed “prey polypeptides” in the context of the presently described selection method of the prey polynucleotides.

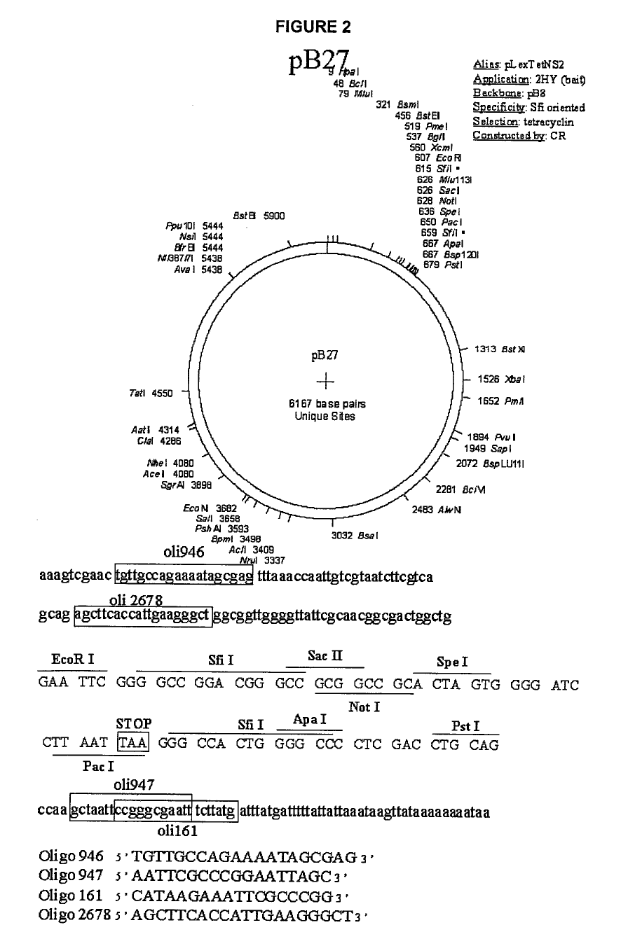

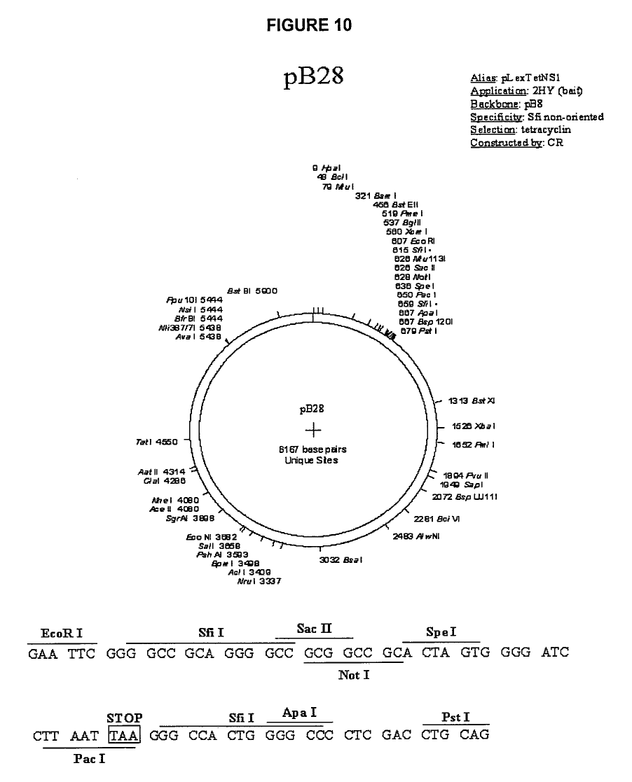

The bait polynucleotides can be inserted in bait plasmid pB27 or pB28 as illustrated in FIG. 2 and FIG. 10, respectively. The bait polynucleotide insert is fused to a polynucleotide encoding the binding domain of, for example, the Gal4 DNA binding domain and the shuttle expression vector is used to transform cells.

The bait polynucleotides used in the present invention are described in Table 1.

As stated above, any cells can be utilized in transforming the bait and prey polynucleotides of the present invention including mammalian cells, bacterial cells, yeast cells, insect cells and the like.

In an embodiment, the present invention identifies protein-protein interactions in yeast. In using known methods a prey positive clone is identified containing a vector which comprises a nucleic acid insert encoding a prey polypeptide which binds to a bait polypeptide of interest. The method in which protein-protein interactions are identified comprises:

i)mating at least one first haploid recombinant yeast cell clone from a recombinant yeast cell clone library that has been transformed with a plasmid containing the prey polynucleotide to be assayed with a second haploid recombinant yeast cell clone transformed with a plasmid containing a bait polynucleotide encoding for the bait polypeptide;

ii)cultivating diploid cell clones obtained in step i) on a selective medium; and

iii)selecting recombinant cell clones which grow on the selective medium.

This method may further comprise:

iv)characterizing the prey polynucleotide contained in each recombinant cell clone which is selected in step iii).

In yet another embodiment of the present invention, in lieu of yeast, Escherichia coli is used in a bacterial two-hybrid system, which encompasses a similar principle to that described above for yeast, but does not involve mating for characterizing the prey polynucleotide.

In yet another embodiment of the present invention, mammalian cells and a method similar to that described above for yeast for characterizing the prey polynucleotide are used.

By performing the yeast, bacterial or mammalian two-hybrid system, it is possible to identify for one particular bait an interacting prey polypeptide. The prey polypeptide that has been selected by testing the library of preys in a screen using the two-hybrid, two plus one hybrid methods and the like, encodes the polypeptide interacting with the protein of interest.

The present invention is also directed, in a general aspect, to a complex of polypeptides, polynucleotides encoding the polypeptides composed of a bait polypeptide or bait polynucleotide encoding the bait polypeptide and a prey polypeptide or prey polynucleotide encoding the prey polypeptide capable of interacting with the bait polypeptide of interest. These complexes are identified in Table 2.

Particular protein-protein interactions and protein complexes identified in the present invention are interactions between PSMD8 and three members of the Dishevelled (DvI) family of proteins, DvI1, 2 and 3. As detained in the background, these interactions are involved in the Wnt pathway and may have numerous applications such as:

1/ the use of PSMD8 expression as a molecular marker for adipogenesis and/or obesity;

2/ the modulation of the expression of PSMD8 to influence cell fate and/or the differentiation of pre-adipocytes and/or adipocytes;

3/ the modulation of the degradation of DvI proteins to influence cell fate and/or differentiation of pre-adipocytes and/or adipocytes;

4/ the modulation of the interaction between DvI proteins and PSMD8 proteins to influence cell fate and/or differentiation of pre-adipocytes and/or adipocytes;

5/ furthermore, the present invention has also allowed the determination of SID of DvI, 2 and 3 protein involved in the interaction, another application may be the use of SIDs of DvI proteins to do the above mentionned modulation.

Modulator compounds of the above mentionned interactions are usefull to cure metabolic diseases such as diabetes, obesity, lipodystrophy and the like.

In another aspect, the present invention relates to a complex of polynucleotides consisting of a first polynucleotide, or a fragment thereof, encoding a prey polypeptide that interacts with a bait polypeptide and a second polynucleotide or a fragment thereof. This fragment has at least about 12 (e.g., at least 12) consecutive nucleotides, but can have between about 12 (e.g., 12) and about 5,000 consecutive nucleotides, or between about 12 and about 10,000 consecutive nucleotides or between about 12 and about 20,000 consecutive nucleotides.

The complexes of the two interacting polypeptides listed in Table 2 and the sets of two polynucleotides encoding these polypeptides also form part of the present invention.

In yet another embodiment, the present invention relates to an isolated complex of at least two polypeptides encoded by two polynucleotides wherein said two polypeptides are associated in the complex by affinity binding and are depicted in columns 1 and 4 of Table 2.

In yet another embodiment, the present invention relates to an isolated complex comprising at least a polypeptide as described in column 1 of Table 2 and a polypeptide as described in column 4 of Table 2. The present invention is not limited to these polypeptide complexes alone but also includes the isolated complex of the two polypeptides in which fragments and/or homologous polypeptides exhibiting at least 95% sequence identity, as well as from 96% sequence identity to 99.99% sequence identity.

Also encompassed in another embodiment of the present invention is an isolated complex in which the SID of the prey polypeptides of SEQ ID NOS: 28, 30, 32 in Table 3 and the odd sequences starting from SEQ ID NOS: 49 to 1173 in column 4 of Table 3 form the isolated complex.

Isolated SID polynucleotides of SEQ ID NOS: 27, 29 or 31 in Table 3 and SID polypeptides of SEQ ID NOS:28, 30 or 32 (Table 3) and the even sequences starting from SEQ ID NOS: 48 to 1172 in column 2 of Table 3 are part of the invention.

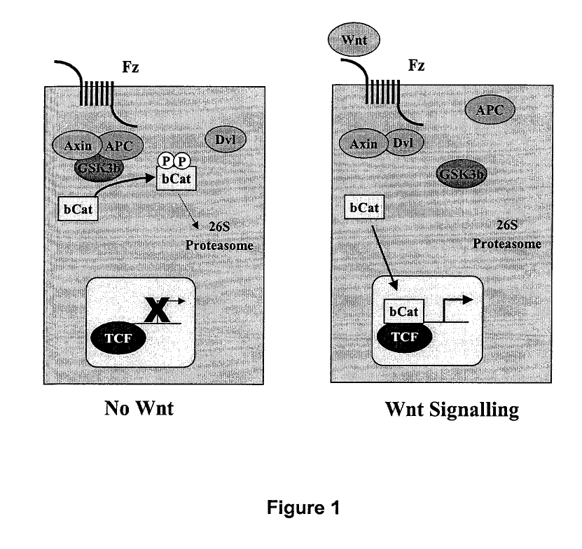

These SIDs, selected interacting domains from Dishevelled proteins 1, 2 and 3 interacting with human PSMD8, are of particular interest since they are involved in the Wnt pathway of adipogenesis (see FIG. 1).

Besides the isolated complexes described above, nucleic acids coding for a Selected Interacting Domain (SID) polypeptide or a variant thereof or any of the nucleic acids set forth in Table 3 can be inserted into an expression vector which contains the necessary elements for the transcription and translation of the inserted protein-coding sequence. Such transcription elements include a regulatory region and a promoter. Thus, the nucleic acid which encodes a marker compound of the present invention is operably linked to a promoter in the expression vector. The expression vector may also include a replication origin.

A wide variety of host/expression vector combinations are employed in expressing the nucleic acids of the present invention. Useful expression vectors that can be used include, for example, segments of chromosomal, non-chromosomal and synthetic DNA sequences.

Suitable vectors include, but are not limited to, derivatives of SV40 and pcDNA and known bacterial plasmids such as col EI, pCR1, pBR322, pMal-C2, pET, pGEX as described by Smith et al Gene, 67:31-40 (1988), pMB9 and derivatives thereof, plasmids such as RP4, phage DNAs such as the numerous derivatives of phage I such as NM989, as well as other phage DNA such as M13 and filamentous single stranded phage DNA; yeast plasmids such as the 2 micron plasmid or derivatives of the 2 m plasmid, as well as centomeric and integrative yeast shuttle vectors; vectors useful in eukaryotic cells such as vectors useful in insect or mammalian cells; vectors derived from combinations of plasmids and phage DNAs, such as plasmids that have been modified to employ phage DNA or the expression control sequences; and the like.

For example in a baculovirus expression system, both non-fusion transfer vectors, such as, but not limited to pVL941 (BamHI cloning site Summers, pVL1393 (BamHI, SmaI, XbaI, EcoRI, NotI, XmaIII, BgIII and PstI cloning sites; Invitrogen) pVL1392 (BgIII, PstI, NotI, XmaIII, EcoRI, XbaII SmaI and BamHI cloning site; Summers and Invitrogen) and pBlueBacIII (BamHI, BgAI, PstI, NcoI and HindIII cloning site, with blue/white recombinant screening, Invitrogen), and fusion transfer vectors such as, but not limited to, pAc700 (BamHI and KpnI cloning sites, in which the BamHI recognition site begins with the initiation codon; Summers), pAc701 and pAc70-2 (same as pAc700, with different reading frames), pAc360 (BamHII cloning site 36 base pairs downstream of a polyhedrin initiation codon; Invitrogen (1995)) and pBlueBacHisA, B, C (three different reading frames with BamHI, BgiIII, PstI, NcoI and HincIII cloning site, an N-terminal peptide for ProBond purification and blue/white recombinant screening of plaques; Invitrogen (220) can be used.

Mammalian expression vectors contemplated for use in the invention include vectors with inducible promoters, such as the dihydrofolate reductase promoters, any expression vector with a DHFR expression cassette or a DHFR/methotrexate co-amplification vector such as pED (PstI, SalI, SbaI, Smal and EcoRI cloning sites, with the vector expressing both the cloned gene and DHFR; Kaufman, 1991). Alternatively a glutamine synthetase/methionine sulfoximine co-amplification vector, such as pEE14 (HindIII, XbalI, SmaI, SbaI, EcoRI and BcII cloning sites in which the vector expresses glutamine synthetase and the cloned gene; Celltech). A vector that directs episomal expression under the control of the Epstein Barr Virus (EBV) or nuclear antigen (EBNA) can be used such as pREP4 (BamHI, SflI, XhoI, NotI, NheI, HindIII, NheI, PvuII and KpnI cloning sites, constitutive RSV-LTR promoter, hygromycin selectable marker; Invitrogen), pCEP4 (BamHI, SflI, XhoI, NolI, NheI, HindIII, NheI, PvuII and KpnI cloning sites, constitutive hCMV immediate early gene promoter, hygromycin selectable marker; Invitrogen), pMEP4 (KpnI, PvuI, NheI, HindIII, NotI, XhoI, SflI, BamHI cloning sites, inducible methallothionein IIa gene promoter, hygromycin selectable marker, Invitrogen), pREP8 (BamHI, XhoI, NotI, HindIII, NheI and KpnI cloning sites, RSV-LTR promoter, histidinol selectable marker; Invitrogen), pREP9 (KpnI, NheI, HindIII, NotI, XhoI, SflI, BamHI cloning sites, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen).

Selectable mammalian expression vectors for use in the invention include, but are not limited to, pRc/CMV (HindIII, BstXI, NotI, SbaI and ApaI cloning sites, G418 selection, Invitrogen), pRc/RSV (HindII, SpeI, BsfXI, NotI, XbaI cloning sites, G418 selection, Invitrogen) and the like. Vaccinia virus mammalian expression vectors (see, for example Kaufman 1991 that can be used in the present invention include, but are not limited to, pSC11 (SmaI cloning site, TK- and β-gal selection), pMJ601 (SalI, SmaI, AflI, NarI, BspMII, BamHI, ApaI, NheI, SacII, KpnI and HindIII cloning sites; TK- and β-gal selection), pTKgptF1S (EcoRI, PstI, SalII, AccI, HindIII, SbaI, BamHI and Hpa cloning sites, TK or XPRT selection) and the like.

Yeast expression systems that can also be used in the present invention include, but are not limited to, the non-fusion pYES2 vector (XbaI, SphI, ShoI, NotI, GstXI, EcoRI, BstXI, BamHI, SacI, KpnI and HindIII cloning sites, Invitrogen), the fusion pYESHisA, B, C (XbaI, SphI, ShoI, NotI, BstXI, EcoRI, BamHI, SacI, KpnI and HindIII cloning sites, N-terminal peptide purified with ProBond resin and cleaved with enterokinase; Invitrogen), pRS vectors and the like.

Consequently, mammalian and typically human cells, as well as bacterial, yeast, fungi, insect, nematode and plant cells an used in the present invention and may be transfected by the nucleic acid or recombinant vector as defined herein.

Examples of suitable cells include, but are not limited to, VERO cells, HELA cells such as ATCC No. CCL2, CHO cell lines such as ATCC No. CCL61, COS cells such as COS-7 cells and ATCC No. CRL 1650 cells, W138, BHK, HepG2, 3T3 such as ATCC No. CRL6361, A549, PC12, K562 cells, 293 cells, Sf9 cells such as ATCC No. CRL1711 and Cv1 cells such as ATCC No. CCL70.

Other suitable cells that can be used in the present invention include, but are not limited to, prokaryotic host cells strains such as Escherichia coli (e.g., strain DH5-α), Bacillus subtilis, Salmonella typhimurium, or strains of the genera of Pseudomonas, Streptomyces and Staphylococcus.

Further suitable cells that can be used in the present invention include yeast cells such as those of Saccharomyces such as Saccharomyces cerevisiae.

Besides the specific isolated complexes, as described above, the present invention relates to and also encompasses Selected Interacting Domain (SID®) polynucleotides. As explained above, for each bait polypeptide, several prey polypeptides can be identified by comparing and selecting the intersection of every isolated fragment that are included in the same polypeptide. Thus the Selected Interacting Domain (SID®) polynucleotides of the present invention are represented by the shared nucleic acid sequences of SEQ ID NOS: 27, 29, 31 in Table 3 and the even sequences starting from SEQ ID NO: 48 to 1172 in column 2 of Table 3 encoding the Selected Interacting Domain (SID®) polypeptides of SEQ ID NOS: 28, 30 or 32 (Table 3) and the odd sequences starting from SEQ ID NO: 49 to 1173 in column 4 of Table 3.

The present invention is not limited to the Selected Interacting Domain (SID®) sequences as described in the above paragraph, but also includes fragments of these sequences having at least about 12 (e.g., at least 12) consecutive nucleic acids, between about 12 (e.g., 12) and about 5,000 consecutive nucleic acids and between about 12 and about 10,000 consecutive nucleic acids and between about 12 and about 20,000 consecutive nucleic acids, as well as variants thereof. The fragments or variants of the SID® sequences possess at least the same affinity of binding to its protein or polypeptide counterpart, against which it has been initially selected. Moreover this variant and/or fragment of the SID® sequences alternatively can have between about 95% (e.g., 95%) and 99.999% sequence identity to its protein or polypeptide counterpart.

According to the present invention variants of polynucleotide or polypeptides can be created by known mutagenesis techniques either in vitro or in vivo. Such a variant can be created such that it has altered binding characteristics with respect to the target protein and more specifically that the variant binds the target sequence with either higher or lower affinity.

Polynucleotides that are complementary to the above sequences which include the polynucleotides of the Selected Interacting Domain's (SID®'s), their fragments, variants and those that have specific sequence identity are also included in the present invention.

The polynucleotide encoding the Selected Interacting Domain (SID®) polypeptide, a fragment or a variant thereof can also be inserted into recombinant vectors which are described in detail above.

The present invention also relates to a composition comprising the above-mentioned recombinant vectors containing the Selected Interacting Domain (SID®) polynucleotides in Table 3, fragments or variants thereof, as well as recombinant host cells transformed by the vectors. The recombinant host cells that can be used in the present invention were discussed in greater detail above.

The compositions comprising the recombinant vectors can contain physiological acceptable carriers such as diluents, adjuvants, excipients and any vehicle in which this composition can be delivered therapeutically and can include, but are not limited to sterile liquids such as water and oils.

Yet another aspect of the present invention relates to a method of selecting modulating compounds, as well as the modulating molecules or compounds themselves which may be used in a pharmaceutical composition. These modulating compounds may act as a cofactor, as an inhibitor, as antibodies, as tags, as a competitive inhibitor, as an activator or alternatively have agonistic or antagonistic activity on the protein-protein interactions.

The activity of the modulating compound does not necessarily, for example, have to be 100% activation or inhibition. Indeed, even partial activation or inhibition can be achieved that is of pharmaceutical interest.

The modulating compound can be selected according to a method which comprises:

cultivating a recombinant host cell containing a reporter gene the expression of which is toxic for said recombinant host cell with a modulating compound on a selective medium and wherein said recombinant host cell is transformed with two vectors:

(i) wherein said first vector comprises a polynucleotide encoding a first hybrid polypeptide having a DNA binding domain;

(ii) wherein said second vector comprises a polynucleotide encoding a second hybrid polypeptide having a transcriptional activating domain that activates said reporter gene when the first and second hybrid polypeptides interact; and

selecting said modulating compound which inhibits or permits the growth of said recombinant host cell.

Thus, the present invention relates to a modulating compound that inhibits the protein-protein interactions of a complex of two polypeptides of columns 1 and 4 of Table 2. The present invention also relates to a modulating compound that activates the protein-protein interactions of a complex of two polypeptides of columns 1 and 4 of Table 2.

In yet another aspect, the present invention relates to a method of identifying such modulating compound, which modulating compound inhibits the interactions of two polypeptides of columns 1 and 4 of Table 2. This method comprises:

(a) cultivating a recombinant host cell and a reporter gene the expression of which is toxic for said recombinant host cell with a modulating compound on a selective medium wherein said recombinant host cell is transformed with two vectors:

(i) wherein said first vector comprises a polynucleotide encoding a first hybrid polypeptide having a first domain of an enzyme;

(ii) wherein said second vector comprises a polynucleotide encoding a second hybrid polypeptide having an enzymatic transcriptional activating domain that activates said toxic reporter gene when the first and second hybrid polypeptides interact; and

(b) selecting said modulating compound which inhibits or permits the growth of said recombinant host cell.

In the two methods described above any toxic reporter gene can be utilized including those reporter genes that can be used for negative selection including the URA3 gene, the CYH1 gene, the CYH2 gene and the like.

In yet another aspect, the present invention provides a kit for screening a modulating compound. This kit comprises a recombinant host cell which comprises a reporter gene the expression of which is toxic for the recombinant host cell. The host cell is transformed with two vectors. The first vector comprises a polynucleotide encoding a first hybrid polypeptide having a DNA binding domain; and a second vector comprises a polynucleotide encoding a second hybrid polypeptide having a transcriptional activating domain that activates said toxic reporter gene when the first and second hybrid polypeptides interact.

In yet another aspect, a kit is provided for screening a modulating compound by providing a recombinant host cell, as described in the paragraph above, but instead of a DNA binding domain, the first vector comprises a first hybrid polypeptide containing a first domain of a protein. The second vector comprises a second polypeptide containing a second part of a complementary domain of a protein that activates the toxic reporter gene when the first and second hybrid polypeptides interact.

In the selection methods described above, the activating domain can be p42 Gal 4, YP16 (HSV) and the DNA-binding domain can be derived from Gal4 or Lex A. The protein or enzyme can be adenylate cyclase, guanylate cyclase, DHFR and the like.

In yet another aspect, the present invention relates to a pharmaceutical composition comprising the modulating compounds for preventing or treating obesity or metabolic diseases in a human or animal, most preferably in a mammal.

This pharmaceutical composition comprises a pharmaceutically acceptable amount of the modulating compound. The pharmaceutically acceptable amount can be estimated from cell culture assays. For example, a dose can be formulated in animal models to achieve a circulating concentration range that includes or encompasses a concentration point or range having the desired effect in an in vitro system. This information can thus be used to accurately determine the doses in other mammals, including humans and animals.

The therapeutically effective dose refers to that amount of the compound that results in amelioration of symptoms in a patient. Toxicity and therapeutic efficacy of such compounds can be determined by standard pharmaceutical procedures in cell cultures or in experimental animals. For example, the LD50 (the dose lethal to 50% of the population) as well as the ED50 (the dose therapeutically effective in 50% of the population) can be determined using methods known in the art. The dose ratio between toxic and therapeutic effects is the therapeutic index which can be expressed as the ratio between LD50 and ED50 compounds that exhibit high therapeutic indexes.

The data obtained from the cell culture and animal studies can be used in formulating a range of dosage of such compounds which lies preferably within a range of circulating concentrations that include the ED50 with little or no toxicity.

The pharmaceutical composition can be administered via any route such as locally, orally, systemically, intravenously, intramuscularly, mucosally, using a patch and can be encapsulated in liposomes, microparticles, microcapsules, and the like. The pharmaceutical composition can be embedded in liposomes or even encapsulated.

Any pharmaceutically acceptable carrier or adjuvant can be used in the pharmaceutical composition. The modulating compound will be preferably in a soluble form combined with a pharmaceutically acceptable carrier. The techniques for formulating and administering these compounds can be found in “Remington's Pharmaceutical Sciences” Mack Publication Co., Easton, Pa., latest edition.

The mode of administration optimum dosages and galenic forms can be determined by the criteria known in the art taken into account the seriousness of the general condition of the mammal, the tolerance of the treatment and the side effects.

The present invention also relates to a method of treating or preventing obesity or metabolic diseases in a human or mammal in need of such treatment. This method comprises administering to a mammal in need of such treatment a pharmaceutically effective amount of a modulating compound which binds to a targeted mammalian or human or adipocyte protein. In a preferred embodiment, the modulating compound is a polynucleotide which may be placed under the control of a regulatory sequence which is functional in the mammal or human.

In yet another aspect, the present invention relates to a pharmaceutical composition comprising a Selected Interacting Domain (SID®) polypeptide, a fragment or a variant thereof. The Selected Interacting Domain (SID®) polypeptide, fragment or variant thereof can be used in a pharmaceutical composition provided that it is endowed with specific binding properties to a bait polypeptide of interest.

The original properties of the Selected Interacting Domain (SID®) polypeptide or variants thereof interfere with the naturally occurring interaction between a first protein and a second protein within the cells of the organism. Thus, the Selected Interacting Domain (SID®) polypeptide binds specifically to either the first polypeptide or the second polypeptide.

Therefore, the Selected Interacting Domain (SID®) polypeptides of the present invention or variants thereof interfere with protein-protein interactions between human proteins.

Thus, the present invention relates to a pharmaceutical composition comprising a pharmaceutically acceptable amount of a Selected Interacting Domain (SID®) polypeptide or variant thereof, provided that the variant has the above-mentioned two characteristics; i.e., that it is endowed with specific binding properties to a bait polypeptide of interest and is devoid of biological activity of the naturally occurring protein.

In yet another embodiment, the present invention relates to a pharmaceutical composition comprising a pharmaceutically effective amount of a polynucleotide encoding a Selected Interacting Domain (SID®) polypeptide or a variant thereof wherein the polynucleotide is placed under the control of an appropriate regulatory sequence. Appropriate regulatory sequences that are used are polynucleotide sequences derived from promoter elements and the like.

Polynucleotides that can be used in the pharmaceutical composition of the present invention include the nucleotide sequences of SEQ ID NOS:. 27, 29 or 31 in Table 3 and the even sequences starting from SEQ ID NOS: 48 to 1172 in column 2 of Table 3.

Besides the Selected Interacting Domain (SID®) polypeptides and polynucleotides, the pharmaceutical composition of the present invention can also include a recombinant expression vector comprising the polynucleotide encoding the SID® polypeptide, a fragment or a variant thereof.

The above described pharmaceutical compositions can be administered by any route such as orally, systemically, intravenously, intramuscularly, intradermally, mucosally, encapsulated, using a patch and the like. Any pharmaceutically acceptable carrier or adjuvant can be used in this pharmaceutical composition.

The Selected Interacting Domain (SID®) polypeptides as active ingredients will be for instance in a soluble form combined with a pharmaceutically acceptable carrier. The techniques for formulating and administering these compounds can be found in “Remington's Pharmaceutical Sciences” supra.

The amount of pharmaceutically acceptable Selected Interacting Domain (SID®) polypeptides can be determined as described above for the modulating compounds using cell culture and animal models.

Such compounds can be used in a pharmaceutical composition to treat or prevent metabolic diseases such as diabetes, obesity, lipodystrophy and the like.

Thus, the present invention also relates to a method of preventing or treating diabetes, obesity, lipodystrophy and the like, said method comprising the steps of administering to a human in need of such treatment a pharmaceutically effective amount of:

(1) a Selected Interacting Domain SID® polypeptide of SEQ ID NOS: 28, 30, 32 in Table 3 and the odd sequences starting from SEQ ID NOS: 49 to 1173 in column 4 of Table 3 or a variant or a fragment thereof which binds to the targeted protein; or

(2) a Selected Interacting Domain (SID®) polynucleotide corresponding to a SID® polypeptide of SEQ ID NOS: 28, 30, 32 in Table 3 and the odd sequences starting from SEQ ID NOS: 49 to 1173 in column 4 of Table 3 or a variant or a fragment thereof wherein said polynucleotide is placed under the control of a regulatory sequence which is functional in said human; or

(3) a recombinant expression vector comprising a polynucleotide encoding a Selected Interacting Domain (SID®) polypeptide which binds to at least one of the disclosed proteins in Table 3.

In another aspect the present invention relates to a method wherein nucleic acids comprising a sequence of SEQ ID NOS: 27, 29 or 31 in Table 3 and the even sequences starting from SEQ ID NOS: 48 to 1172 in column 2 of Table 3 which encodes the protein of sequence SEQ ID NOS: 28, 30, 32 in Table 3 and the odd sequences starting from SEQ ID NOS: 49 to 1173 in column 4 of Table 3 and/or functional derivatives thereof are administered to modulate complex (from Table 2) function by way of gene therapy. Any of the methodologies relating to gene therapy available within the art can be used in the practice of the present invention such as those described by Goldspiel et al Clin. Pharm. 12 pgs. 488-505 (1993).

The above described pharmaceutical compositions can be administered by any route such as orally, systemically, intravenously, intramuscularly, intradermally, mucosally, encapsulated, using a patch and the like. Any pharmaceutically acceptable carrier or adjuvant can be used in this pharmaceutical composition.

Delivery of the therapeutic nucleic acid into a patient may be direct in vivo gene therapy (i.e., the patient is directly exposed to the nucleic acid or nucleic acid-containing vector) or indirect ex vivo gene therapy (i.e., cells are first transformed with the nucleic acid in vitro and then transplanted into the patient).

For example for in vivo gene therapy, an expression vector containing the nucleic acid is administered in such a manner that it becomes intracellular; i.e., by infection using a defective or attenuated retroviral or other viral vectors as described, for example in U.S. Pat. No. 4,980,286 or by Robbins et al, Pharmacol. Ther., 80 No. 1 pgs. 35-47 (1998).

The various retroviral vectors that are known in the art are such as those described in Miller et al. (Meth. Enzymol. 217pgs. 581-599 (1993)) which have been modified to delete those retroviral sequences which are not required for packaging of the viral genome and subsequent integration into host cell DNA. Also adenoviral vectors can be used which are advantageous due to their ability to infect non-dividing cells and such high-capacity adenoviral vectors are described in Kochanek (Human Gene Therapy, 10, pgs. 2451-2459 (1999)). Chimeric viral vectors that can be used are those described by Reynolds et al. (Molecular Medicine Today, pgs. 25 -31 (1999)). Hybrid vectors can also be used and are described by Jacoby et al. (Gene Therapy, 4, pgs. 1282-1283 (1997)).

Direct injection of naked DNA or through the use of microparticle bombardment (e.g., Gene Gun®; Biolistic, Dupont) or by coating it with lipids can also be used in gene therapy. Cell-surface receptors/transfecting agents or through encapsulation in liposomes, microparticles or microcapsules or by administering the nucleic acid in linkage to a peptide which is known to enter the nucleus or by administering it in linkage to a ligand predisposed to receptor-mediated endocytosis (See Wu & Wu, J. Biol. Chem., 262 pgs. 4429-4432 (1987)) can be used to target cell types which specifically express the receptors of interest.

In another embodiment a nucleic acid ligand compound may be produced in which the ligand comprises a fusogenic viral peptide designed so as to disrupt endosomes, thus allowing the nucleic acid to avoid subsequent lysosomal degradation. The nucleic acid may be targeted in vivo for cell specific endocytosis and expression by targeting a specific receptor such as that described in WO92/06180, WO93/14188 and WO93/20221. Alternatively the nucleic acid may be introduced intracellularly and incorporated within the host cell genome for expression by homologous recombination (See Zijistra et al, Nature, 342, pgs. 435-428 (1989)).

In ex vivo gene therapy, a gene is transferred into cells in vitro using tissue culture and the cells are delivered to the patient by various methods such as injecting subcutaneously, application of the cells into a skin graft and the intravenous injection of recombinant blood cells such as hematopoietic stem or progenitor cells.

Cells into which a nucleic acid can be introduced for the purposes of gene therapy include, for example, epithelial cells, endothelial cells, keratinocytes, fibroblasts, muscle cells, hepatocytes and blood cells. The blood cells that can be used include, for example, T-lymphocytes B-lymphocytes, monocytes, macrophages, neutrophils, eosinophils, megakaryotcytes, granulocytes, hematopoietic cells or progenitor cells and the like.

In yet another embodiment the present invention relates to protein chips or protein microarrays. It is well known in the art that microarrays can contain more than 10,000 spots of a protein that can be robotically deposited on a surface of a glass slide or nylon filter. The proteins attach covalently to the slide surface, yet retain their ability to interact with other proteins or small molecules in solution. In some instances the protein samples can be made to adhere to glass slides by coating the slides with an aldehyde-containing reagent that attaches to primary amines. A process for creating microarrays is described, for example by MacBeath and Schreiber (Science, Volume 289, Number 5485, pgs, 1760-1763 (2000)) or (Service, Science, Vol, 289, Number 5485 pg. 1673 (2000)). An apparatus for controlling, dispensing and measuring small quantities of fluid is described, for example, in U.S. Pat. No. 6,112,605.

The present invention also provides a record of protein-protein interactions, PIM's and any data encompassed in the following Tables. It will be appreciated that this record can be provided in paper or electronic or digital form.

In order to fully illustrate the present invention and advantages thereof, the following specific examples are given, it being understood that the same are intended only as illustrative and in no way limitative. The two adipocytes strains (undifferentiated and differentiated PAZ-6 adipocytes) studied in the present invention are obtained by the method described in the PCT patent application WO96/34100 but products of the present invention may also be used for any other adipocyte strain.

### EXAMPLES

### Example 1

Comparison of cDNA Expression Profiles in Differentiated and Undifferentiated Adipocytes

A Megasort analysis of the two RNA samples from undifferentiated and differentiated PAZ6 cells has been performed two times according to the protocol described in Brenner et al. (Brenner, S., Williams, S. R., Vermaas, E. H., Storck, T., Moon, K., McCollum, C., Mao, J., Luo, S., Kirchner, J. J., Eletr, S., DuBridge, R. B., Burcham, T., and Albrecht, G., In vitro cloning of complex mixtures of DNA microbeads: physical separation of differentially expressed cDNA, PNAS, 97(4), 1665-1670, 2000).

1.A. Library Preparation

Four libraries have been prepared:

an Oligonucleotide Tag Library and a Probe Library with 2 μg of polyA+ RNA from differentiated PAZ6 adipocytes; and

an Oligonucleotide Tag Library and a Probe Library with 2 μg of polyA+ RNA from undifferentiated PAZ6 adipocytes.

The two Oligonucleotide Tag Libraries are mixed together leading to a global Tag Library with a total of 600,000 microbeads.

1.B. Hybridization Test

Each Probe Library, differentiated and undifferentiated adipocytes, was tested against the global Tag Library.

1. C. Analysis of the Results

By analysis of the obtained differential gene expression profiles, cDNA specifically over-expressed in differentiated adipocytes have been selected in order to be clone as prey in two-hybrid bait vector (pB27):

HSTUMP (SEQ ID NO: 5 and 6, Genbank ref|NM_{—}003295.1| Homo sapiens tumor protein, translationally-controlled 1 (TPT1));

Leptin (SEQ ID NO: 9 and 10, Genbank ref|NM_{—}000230.1| Homo sapiens leptin);

C1R (Genbank ref|NM_{—}001733.1| Homo sapiens complement component 1, r subcomponent);

Thymosin beta-4 (Genbank ref|NM_{—}021109.1| Homo sapiens thymosin, beta 4, X chromosome (TMSB4X));

Fibulin 1C (SEQ ID NO: 13 and 14, Genbank ref|NM_{—}001996.1| Homo sapiens fibulin 1 (FBLN1), transcript variant C);

SPARC/Osteonectin (SEQ ID NO: 1 and 2, Genbank ref|NM_{—}003118.1| Homo sapiens secreted protein, acidic, cysteine-rich),

Beta 2 microglobulin (SEQ ID NO: 7 and 8, Genbank ref|NM_{—}004048.1| Homo sapiens beta-2-microglobulin),

Human proteasome subunit p31 (SEQ ID NO: 15 and 16, Genbank ref|NM_{—}002812.1| Homo sapiens proteasome (prosome, macropain) 26S subunit, non-ATPase, 8 (PSMD8));

Human interferon-inducible peptide (6-16) gene (SEQ ID NO: 21 and 22, 23 and 24 or 25 and 26, Genbank gb|U22970.1| HSU22970);

Huntingtin-interacting protein 2 (SEQ ID NO: 11 and 12, genbank ref|NM_{—}005339.21|)

and Human 1-8U gene from interferon-inducible gene family (SEQ ID NO: 17 and 18, Genbank ref|NM_{—}021034.1|).

For the bait sequence of SEQ ID NO: 42 to 47 (see Table 1), pB28 was used as bait plasmid.

### Example 2

Preparation of a Collection of Random-Primed CDNA Fragments

2.A. Collection Preparation and Transformation in Escherichia coli

2.A.1. Random-Primed cDNA Fragment Preparation

For mRNA sample from differentiated PAZ6 adipocytes, random-primed cDNA was prepared from 5 μg of polyA+ mRNA using a TimeSaver cDNA Synthesis Kit (Amersham Pharmacia Biotech) and with 5 μg of random N9-mers according to the manufacturer's instructions. Following phenolic extraction, the cDNA was precipitated and resuspended in water. The resuspended cDNA was phosphorylated by incubating in the presence of T4 DNA Kinase (Biolabs) and ATP for 30 minutes at 37° C. The resulting phosphorylated cDNA was then purified over a separation column (Chromaspin TE 400, Clontech), according to the manufacturer's protocol.

2.A.2. Ligation of Linkers to Blunt-Ended cDNA

Oligonucleotide HGX931 (5′-end phosphorylated) 1 μg/μl and HGX932 μg/μl.

Sequence of the oligo HGX931: 5′-GGGCCACGAA-3′ (SEQ ID NO: 35)

Sequence of the oligo HGX932: 5′-TTCGTGGCCCCTG-3′ (SEQ ID NO: 36)

Linkers were preincubated (5 minutes at 95° C., 10 minutes at 68° C., 15 minutes at 42° C.) then cooled down at room temperature and ligated with cDNA fragments at 16° C. overnight.

Linkers were removed on a separation column (ChromaspinTE400, Clontech), according to the manufacturer's protocol.

2.A.3. Vector Preparation

Plasmid pP6 (see FIG. 4) was prepared by replacing the Spel/XhoI fragment of pGAD3S2X with the double-stranded oligonucleotide:

(SEQ ID NO: 37)

5′-CTAGCCATGGCCGCAGGGGCCGCGGCCGCACTAGTGGGGATCCTTAA
TTAAAGGGCCACTGGGGCCCCCGGTACCGGCGTCCCCGGCGCCGGCGTGA
TCACCCCTAGGAATTAATTTCCCGGTGACCCCGGGGGAGCT-3′

The pP6 vector was successively digested with Sfi1 and BamHI restriction enzymes (Biolabs) for 1 hour at 37° C., extracted, precipitated and resuspended in water. Digested plasmid vector backbones were purified on a separation column (Chromaspin TE 400, Clontech), according to the manufacturer's protocol.

2.A.4. Ligation Between Vector and Insert of CDNA

The prepared vector was ligated overnight at 15° C. with the blunt-ended cDNA described in section 2 using T4 DNA ligase (Biolabs). The DNA was then precipitated and resuspended in water.

2.A.5. Library Transformation in Escherichia coli

The DNA from section 1.A.4 was transformed into Electromax DH10B electrocompetent cells (Gibco BRL) with a Cell Porator apparatus (Gibco BRL). 1 ml SOC medium was added and the transformed cells were incubated at 37° C. for 1 hour. 9 mis of SOC medium per tube was added and the cells were plated on LB+ampicillin medium. The colonies were scraped with liquid LB medium, aliquoted and frozen at −80°C.

The obtained collection of recombinant cell clones was named HGXBPZDRP1.

2.B. Collection Transformation in Saccharomyces cerevisiae

The Saccharomyces cerevisiae strain (Y187 (MATαGal4Δ Gal8Δ ade2-1 01, his3, leu2-3, −112, trpl-901, ura3-52 URA3::UASGAL1-LacZ Met)) was transformed with the cDNA library.

The plasmid DNA contained in E. coli were extracted (Qiagen) from aliquoted E. coli frozen cells (1.A.5.). Saccharomyces cerevisiae yeast Y187 in YPGlu were grown.

Yeast transformation was performed according to standard protocol (Giest et al. Yeast, 11, 355-360, 1995) using yeast carrier DNA (Clontech). This experiment leads to 10⁴ to 5×10⁴ cells/μg DNA. 2×10⁴ cells were spread on DO-Leu medium per plate. The cells were aliquoted into vials containing 1 ml of cells and frozen at −80° C.

The obtained collection of recombinant cell clones was named HGXYPZDRP1.

2.C. Construction of Bait Plasmids

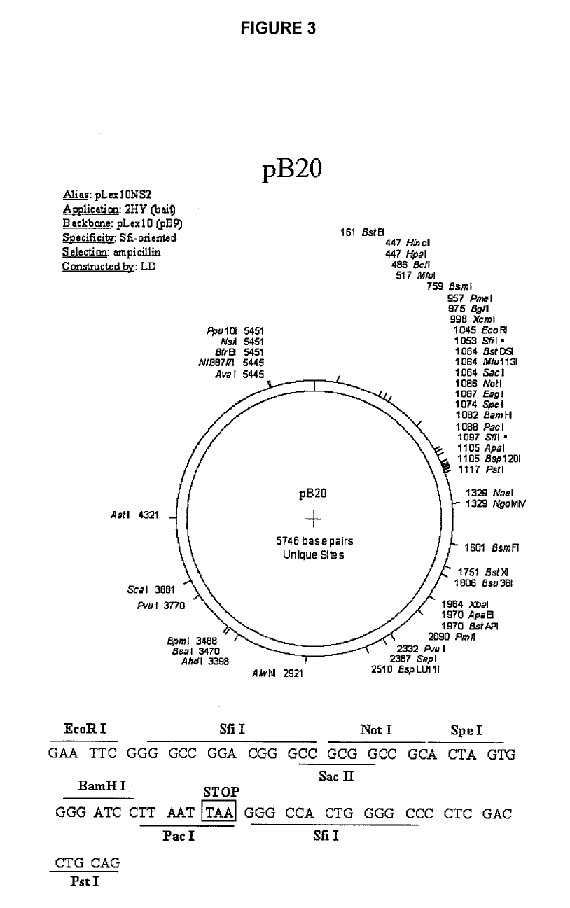

For fusions of the bait protein (see selection of bait proteins in section 1.C.) to the DNA-binding domain of the GAL4 protein of S. cerevisiae, bait fragments were cloned into plasmid pB27 (see FIG. 2). For fusions of the bait protein to the DNA-binding domain of the LexA protein of E. coli, bait fragments were cloned into plasmid pB20 (see FIG. 3).

Plasmid pB27 was prepared by replacing the Nco1/Sal1 polylinker fragment of pAS≢Δ with the double-stranded DNA fragment:

(SEQ ID NO: 38)

5′ CATGGCCGGACGGGCCGCGGCCGCACTAGTGGGGATCCTTAATTAAA
GGGCCACTGGGGCCCCC 3′

(SEQ ID NO: 39)

3′ CGGCCTGCCCGGCGCCGGCGTGATCACCCCTAGGAATTAATTTCCCG
GTGACCCCGGGGGAGCT 5′

Plasmid pB20 was prepared by replacing the EcoRIPsfI polylinker fragment of pLex10 with the double-stranded DNA fragment:

(SEQ ID NO: 40)

5′ AATTCGGGGCCGGACGGGCCGCGGCCGCACTAGTGGGGATCCTTAAT
TAAGGGCCACTGGGGCCCCTCGACCTGCA 3′

(SEQ ID NO: 41)

3′ GCCCCGGCCTGCCCGGCGCCGGCGTGATCACCCCTAGGAATTAATTC
CCGGTGACCCCGGGGAGCTGG 5′

Plasmid pB28 was prepared by replacing the EcoRI/PstI polylinker fragment of pB27 with the double stranded DNA fragment:

(SEQ ID NO: 1174)

5′ GAATTCGGGGCCGCAGGGGCCGCGGCCGCACTAGTGGGGATCCTTAATTA
AGGGCCACTGGGGCCCCTCGACCTGCAG 3′

(SEQ ID NO: 1175)

5′ CTGCAGGTCGAGGGGCCCCAGTGGCCCTTAATTAAGGATCCCCACTAGTGC
GGCCGCGGCCCCTGCGGCCCCGAATTC 3′

The amplification of the bait ORF was obtained by PCR using the Pfu proof-reading Taq polymerase (Stratagene), 10 pmol of each specific amplification primer and 200 ng of plasmid DNA as template.

The PCR program was set up as follows:

$$\begin{matrix} 94 {°45}^{″} \\ \begin{matrix}94 {°45}^{″} \\ 48 {°45}^{″} \times 30 cycles \\ 72 {°6}^{′}\end{matrix} \} \\ 72 {°10}^{′} \\ 15 °∞\end{matrix}$$

The amplification was checked by agarose gel electrophoresis.

The PCR fragments were purified with Qiaquick column (Qiagen) according to the manufacturer's protocol.

Purified PCR fragments were digested with adequate restriction enzymes.

The PCR fragments were purified with Qiaquick column (Qiagen) according to the manufacturer's protocol.

The digested PCR fragments were ligated into an adequately digested and dephosphorylated bait vector (pB27 or pB20) according to standard protocol (Sambrook et al.) and were transformed into competent bacterial cells. The cells were grown, the DNA extracted and the plasmid was sequenced.

### Example 3

Screening the Collection With the Two-Hybrid in Yeast System

3.A. The Mating Protocol

The mating two-hybrid in yeast system (as described by Legrain et al., Nature Genetics, vol. 16, 277-282 (1997), Toward a functional analysis of the yeast genome through exhaustive two-hybrid screens) was used for its advantages but one could also screen the cDNA collection in classical two-hybrid system as described in Fields et al. or in a yeast reverse two-hybrid system.

The mating procedure allows a direct selection on selective plates because the two fusion proteins are already produced in the parental cells. No replica plating is required.

This protocol was written for the use of the library transformed into the Y187 strain.

For bait proteins fused to the DNA-binding domain of GAL4, bait-encoding plasmids were first transformed into S. cerevisiae (CG1945 strain (MATa Gal4-542 Gall 80-538 ade2-101 his3Δ200, leu2-3, 112, trp1-901, ura3-52, lys2-801, URA3::GAL4 17mer (X3)-CyC1TATA-LacZ, LYS2::GAL1UAS-GAL1TATA-HIS3 CYH^{R})) according to step 2.B. and spread on DO-Trp medium.

For bait proteins fused to the DNA-binding domain of LexA, bait-encoding plasmids were first transformed into S. cerevisiae (L40Δgal4 strain (MATa ade2, trp1-901, leu2 3,112, lys2-801, his3Δ200, LYS2::(lexAop)₄-HIS3, ura3-52::URA3 (lexAop)₈-LacZ, GAL4::Kan^{R})) according to step 2.B. and spread on DO-Trp medium.

Day 1, Morning: Preculture

The cells carrying the bait plasmid obtained at step 2.C. were precultured in 20 ml DO-Trp medium and grown at 30° with vigorous agitation.

Day 1, Late Afternoon: Culture

The OD_{600 nm} of the DO-Trp pre-culture of cells carrying the bait plasmid pre-culture was measured. The OD_{600 nm} must lie between 0.1 and 0.5 in order to correspond to a linear measurement.

50 ml DO-Trp at OD_{600 nm} 0.006/ml was inoculated and grown overnight at 30° C. with vigorous agitation.

Day 2: Mating Medium and Plates

1 YPGlu 15 cm plate

50 ml tube with 13 ml DO-Leu-Trp-His

100 ml flask with 5 ml of YPGlu

8 DO-Leu-Trp-His plates

2 DO-Leu plates

2 DO-Trp plates

2 DO-Leu-Trp plates

The OD_{600 nm} of the DO-Trp culture was measured. It should be around 1. For the mating, twice as many bait cells as library cells were used. To get a good mating efficiency, one must collect the cells at 10⁸ cells per cm².

The amount of bait culture (in ml) that makes up 50 OD_{600 nm} units for the mating with the prey library was estimated.

A vial containing the HGXYPZDRP1 library was thawed slowly on ice. 1.0 ml of the vial was added to 5 ml YPGlu. Those cells were recovered at 30° C., under gentle agitation for 10 minutes.

Mating

The 50 OD_{600 nm} units of bait culture was placed into a 50 ml falcon tube.

The HGXYPZDRP1 library culture was added to the bait culture, then centrifuged, the supernatant discarded and resuspended in 1.6 ml YPGlu medium.

The cells were distributed onto two 15 cm YPGlu plates with glass beads. The cells were spread by shaking the plates. The plate cells-up at 30° C. for 4 h 30 min were incubated.

Collection of Mated Cells

The plates were washed and rinsed with 6 ml and 7 ml respectively of DO-Leu-Trp-His. Two parallel serial ten-fold dilutions were performed in 500 μl DO-Leu-Trp-His up to 1/10,000. 50 μl of each 1/10000 dilution was spread onto DO-Leu and DO-trp plates and 50 μi of each 1/1000 dilution onto DO-Leu-Trp plates. 22.4 ml of collected cells were spread in 400 μl aliquots on DO-Leu-Trp-His+Tet plates.

Day 4

Clones that were able to grow on DO-Leu-Trp-His+Tetracyclin were then selected. This medium allows one to isolate diploid clones presenting an interaction.

The His+colonies were counted on control plates.

The number of His+cell clones will define which protocol is to be processed

Upon 60.106 Trp+Leu+colonies:

if the number His+cell clones <285: then use the process luminometry protocol on all colonies

if the number of His+cell clones >285 and <5000: then process via overlay and then luminometry protocols on blue colonies (3.B and 3.C).

if number of His+cell clones >5000: repeat screen using DO-Leu-Trp-His+Tetracyclin plates containing 3-aminotriazol.

3. B. The X-Gal Overlay Assay

The X-Gal overlay assay was performed directly on the selective medium plates after scoring the number of His+colonies.

Materials

A waterbath was set up. The water temperature should be 50° C.

0.5 M Na₂HPO₄ pH 7.5.

1.2% Bacto-agar.

2% X-Gal in DMF.

Overlay mixture: 0.25 M Na₂HPO₄ pH7.5, 0.5% agar, 0.1% SDS, 7% DMF (LABOSI), 0.04% X-Gal (ICN). For each plate, 10 ml overlay mixture are needed.

DO-Leu-Trp-His plates.

Sterile toothpicks.

Experiment

The temperature of the overlay mix should be between 45° C. and 50° C. The overlay-mix was poured over the plates in portions of 10 ml. When the top layer was settled, they were collected. The plates were incubated overlay-up at 30° C. and the time was noted. Blue colonies were checked for regularly. If no blue colony appeared, overnight incubation was performed.

Using a pen the number of positives was marked. The positives colonies were streaked on fresh DO-Leu-Trp-His plates with a sterile toothpick.

Either the luminometry or the stamp-overlay assay was next performed.

3. C. The Luminometry Assay

His+colonies were grown overnight at 30° C, in microtiter plates containing DO-Leu-Trp-His+Tetracyclin medium with shaking. The day after, the overnight culture was diluted 15 times into a new microtiter plate containing the same medium and was incubated for 5 hours at 30° C. with shaking. The samples were diluted 5 times and read OD_{600 nm}. The samples were diluted again to obtain between 10,000 and 75,000 yeast cells/well in 100 μl final volume.

Per well, 76,μl of One Step Yeast Lysis Buffer (Tropix) was added, 20 μl Sapphirell Enhancer (Tropix), 4 μl Galacton Star (Tropix) and incubated 40 minutes at 300° C. The β-Gal read-out (L) was measured using a Luminometer (Trilux, Wallach). The value of (OD_{600 nm}×L) was calculated and interacting preys having the highest values were selected.

At this step of the protocol, diploid cell clones presenting interaction were isolated. The next step was now to identify polypeptides involved in the selected interactions.

3.C. The Stamp Overlay Assay

His+colonies were grown overnight at 30° C. in microtiter plates containing DO-Leu-Trp-His+Tetracyclin medium with shaking. The day after the overnight culture, the 96 colonies were stamped on a 15 cm plate of DO-Leu-Trp-His. 4 control yeast colonies were spotted on the same plate. After 2 days of growing at 30° C., an overlay assay was performed on this plate with 80 ml of overlay mixture (see step 2.B.). After 2 hours of incubation, the plate was photographed with a CCD camera. The blue intensity was quantified by Genetool® software (SYNGENE) and normalized to the control spots.

### Example 4

Identification of Positive Clones

4.A. PCR on Yeast Colonies

Introduction

PCR amplification of fragments of plasmid DNA directly on yeast colonies is a quick and efficient procedure to identify sequences cloned into this plasmid. It is directly derived from a published protocol (Wang H. et al., Analytical Biochemistry, 237, 145-146, (1996)). However, it is not a standardized protocol and it varies from strain to strain and it is dependent of experimental conditions (number of cells, Taq polymerase source, etc). This protocol should be optimized to specific local conditions.

Materials

For 1 well, PCR mix composition was:

32.5 μl water,

5 μl 10× PCR buffer (Pharmacia),

1μL dNTP 10 mM,

0.5 μl Taq polymerase (5μ/μl) (Pharmacia),

0.5 μl oligonucleotide ABS1 10 pmole/μl: 5′-GCGTTTGGAATCACTACAGG-3′,(SEQ ID NO: 33)

0.5 μl oligonucleotide ABS2 10 pmole/μI: 5′-CACGATGCACGTTGAAGTG-3′.(SEQ ID NO: 34)

−1 N NaOH.

Experiment

The positive colonies were grown overnight at 30° C. on a 96 well cell culture cluster (Costar), containing 150 μl DO-Leu-Trp-His+Tetracyclin with shaking. The culture was resuspended and 100 μl was transferred immediately on a Thermowell 96 (Costar) and centrifuged for 5 minutes at 4,000 rpm at room temperature. The supernatant was removed. 5 μl NaOH was added to each well and shaken for 1 minute.

The Thermowell was placed in the thermocycler (GeneAmp 9700, Perkin Elmer) for 5 minutes at 99.9° C. and then 10 minutes at 4° C. In each well, the PCR mix was added and shaken well.

The PCR program was set up as followed

$$\begin{matrix} 94 ° C . 3 minutes \\ \begin{matrix}94 ° C . 30 seconds \\ 53 ° C . 1 minute 30 seconds \times 35 cycles \\ 72 ° C . 3 minutes\end{matrix} \} \\ 72 ° C . 5 minutes \\ 15 ° C . ∞\end{matrix}$$

The quality, the quantity and the length of the PCR fragment was checked on an agarose gel. The length of the cloned fragment was the estimated length of the PCR fragment minus 300 base pairs that corresponded to the amplified flanking plasmid sequences.

4.B. Plasmids Rescue From Yeast by Electroporation

Introduction

The previous protocol of PCR on yeast cell may not be successful, in such a case, plasmids from yeast by electroporation can be rescued. This experiment allows the recovery of prey plasmids from yeast cells by transformation of E. Coli with a yeast cellular extract. The prey plasmid can then be amplified and the cloned fragment can be sequenced.

Materials

Plasmid Rescue

Glass beads 425-600 μm (Sigma)

Phenol/chloroform (1/1) premixed with isoamyl alcohol (Amresco)

Extraction buffer: 2% Triton ×100, 1% SDS, 100 mM NaCl, 10 mM TrisHCI pH 8.0,1 mM

EDTA pH 8.0.

Mix ethanol/NH₄Ac: 6 volumes ethanol with 7.5 M NH₄ acetate, 70% ethanol and yeast cells in patches on plates.

Electroporation

SOC medium

M9 medium

Selective plates: M9-Leu+Ampicillin

2 mm electroporation cuvettes (Eurogentech)

Experiment

Plasmid Rescue

The cell patch on DO-Leu-Trp-His was prepared with the cell culture of section 2.C. The cell of each patch was scraped into an Eppendorf tube, 300 μl of glass beads was added in each tube, then, 200 μl extraction buffer and 200 μl phenol:chloroform:isoamyl alcohol (25:24:1) were added.

The tubes were centrifuged for 10 minutes at 15,000 rpm.

180 μl supernatant was transferred to a sterile Eppendorf tube and 500 μl each of ethanol/NH₄Ac was added and the tubes were vortexed. The tubes were centrifuged for 15 minutes at 15,000 rpm at 4° C. The pellet was washed with 200 μl 70% ethanol and the ethanol was removed and the pellet was dried. The pellet was resuspended in 10 μl water. Extracts were stored at −20° C.

Electroporation

Materials: Electrocompetent MC1066 cells prepared according to standard protocols (Sambrook et al., supra).

1 μl of yeast plasmid DNA-extract was added to a pre-chilled Eppendorf tube, and kept on ice.

1 μl plasmid yeast DNA-extract sample was mixed and 20 μl electrocompetent cells was added and transferred in a cold electroporation cuvette.

Set the Biorad electroporator on 200 ohms resistance, 25 μF capacity; 2.5 kV. Place the cuvette in the cuvette holder and electroporate.

1 ml of SOC was added into the cuvette and the cell-mix was transferred into a sterile Eppendorf tube. The cells were recovered for 30 minutes at 37° C., then spun down for 1 minute at 4,000×g and the supernatant was poured off. About 100 μl medium were kept and used to resuspend the cells and spread them on selective plates (e.g., M9-Leu plates). The plates were then incubated for 36 hours at 37° C.

One colony was grown and the plasmids were extracted. Check for the presence and size of the insert through enzymatic digestion and agarose gel electrophoresis. The insert was then sequenced.

Results are shown in Table 1.

### Example 5

Protein-Protein Interaction

For each bait, the previous protocol leads to the identification of prey polynucleotide sequences. Using a suitable software program (e.g., Blastwun, available on the Internet site of the University of Washington: httD://bioweb.pasteur.fr/secianal/interfaces/blastwu.html) the identity of the mRNA transcript that is encoded by the prey fragment may be determined and whether the fusion protein encoded is in the same open reading frame of translation as the predicted protein or not.

Alternatively, prey nucleotide sequences can be compared with one another and those which share identity over a significant region (60 nt) can be grouped together to form a contiguous sequence (Contig) whose identity can be ascertained in the same manner as for individual prey fragments described above.

### Example 6

Identification of SID®

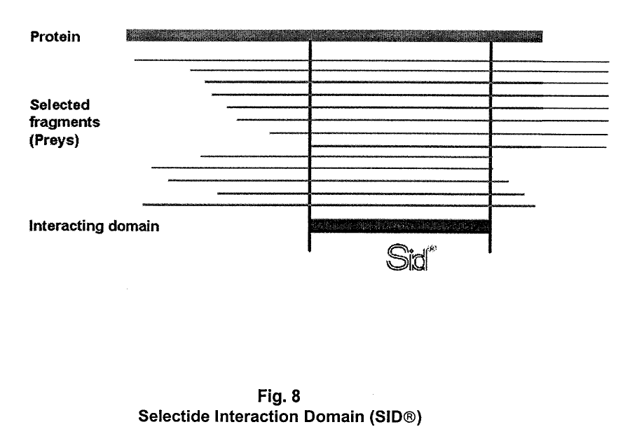

By comparing and selecting the intersection of all isolated fragments that are included in the same polypeptide, one can define the Selected Interacting Domain (SID) is determined as illustrated in FIG. 8. The obtained SIDs are illustrated in Table 3.

### Example 7

Modulating Compounds Identification

Each specific protein-protein complex of columns 1 and 4 of Table 2 may be used to screen for modulating compounds.

One appropriate construction for this modulating compound screening is:

bait polynucleotide inserted in pB27;

prey polynucleotide inserted in pP6;

transformation of these two vectors in a permeable yeast cell;

growth of the transformed yeast cell on a medium containing the modulating compound to be tested,

and observation of the growth of the yeast cells.

### Example 8

1 “Rebound ” screens:

Additional yeast two-hybrid screens were carried out to investigate further the interaction between PSDM8 and Dishevelled 1, 2 and 3 (DVL1, 2 and 3). In particular, these tests were performed to determine whether these interactions could be detected in the reciprocal bait to prey orientation. For this purpose, DVL 1, 2 and 3 prey fragments (see Table 1) were transferred into the pB28 bait vector and screened against the human placenta (random- and oligo dT-primed) and differentiated PAZ6 cell line (random-primed) libraries. As shown in Tables 2 and 3, DVL1, 2 and 3 were found to interact with PSDM8 in these “rebounds” screens. Taken together, these data considerably enforce the DVUPSDM8 interaction (Rain et al, Nature 2001, 409 :211-15; Walhout et al., Science. 2000, 287:116-22).

2-MPSS Analysis:

Using the MegaSort technology (designed to identify differentially expressed cDNAs; Brenner et al., PNAS 2000, 97 :1665-70), PSDM8 was identified as a gene that is specifically over-expressed in differentiated PAZ-6 adipocytes when compared with undifferentiated PAZ-6 preadipocytes.

To confirm this initial observation, gene expression analysis was performed on mRNA samples isolated from pre- and adipocytes PAZ-6 cells using the MPSS (for Massively Parallel Signature Sequencing) technology (Brenner et al., Nature Biotech 2000, 18 :630-4). Using this independent approach, PSDM8 was found to be induced 2.45-fold in mature adipocyte when compare to pre-adipocyte, confirming the initial observation.

### Example 9

Making of Polyclonal and Monoclonal Antibodies

The protein-protein complex of columns 1 and 4 of Table 2 is injected into mice and polyclonal and monoclonal antibodies are made following the procedure set forth in Sambrook et al supra.

More specifically, mice are immunized with an immunogen comprising the above mentioned complexes conjugated to keyhole limpet hemocyanin using glutaraldehyde or EDC as is well known in the art. The complexes can also be stabilized by crosslinking as described in WO00/37483. The immunogen is then mixed with an adjuvant. Each mouse receives four injections of 10 μg to 100 μg of immunogen, and after the fourth injection, blood samples are taken from the mice to determine if the serum contains antibodies to the immunogen. Serum titer is determined by ELISA or RIA. Mice with sera indicating the presence of antibody to the immunogen are selected for hybridoma production.

Spleens are removed from immune mice and single-cell suspension is prepared (Harlow et al 1988). Cell fusions are performed essentially as described by Kohler et al. Briefly, P365.3 myeloma cells (ATTC Rockville, Md.) or NS-1 myeloma cells are fused with spleen cells using polyethylene glycol as described by Harlow et al (1989). Cells are plated at a density of 2×10⁵ cells/well in 96-well tissue culture plates. Individual wells are examined for growth and the supernatants of wells with growth are tested for the presence of complex-specific antibodies by ELISA or RIA using the protein-protein complex of columns 1 and 4 of Table 2 as a target protein. Cells in positive wells are expanded and subcloned to establish and confirm monoclonality.

Clones with the desired specificities are expanded and grown as ascites in mice or in a hollow fiber system to produce sufficient quantities of antibodies for characterization and assay development. Antibodies are tested for binding to bait polypeptide of column 1 of Table 2 alone or to prey polypeptide of column 4 of Table 2 alone, to determine which are specific for the protein-protein complex of columns 1 and 4 of Table 2 as opposed to those that bind to the individual proteins.

Monoclonal antibodies against each of the complexes set forth in columns 1 and 4 of Table 2 are prepared in a similar manner by mixing specified proteins together, immunizing an animal, fusing spleen cells with myeloma cells and isolating clones which produce antibodies specific for the protein complex, but not for individual proteins.

The following results obtained from these Examples, as well as the teachings in the specification are set forth in the Tables below.

All patent and non-patent publications, as well as non-patented websites cited in the present specification are incorporated herein by reference as if each were individually incorporated by reference.

While the invention has been described in terms of the various preferred embodiments, the skilled artisan will appreciate that various modifications, substitutions, omissions and changes may be made without departing from the scope thereof. Accordingly, it is intended that the present invention be limited by the scope of the following claims, including equivalents thereof.

TABLE 1

bait names and sequences

| | | | | |
|---|---|---|---|---|
| | 2. Bait | | | |
| | acid | | 4. Bait | |
| | nucleic | | Aminoacid | |
| | SEQ ID | | SEQ ID | 5. Bait aminoacid |
| 1. Bait Name | number | 3. Bait acid nucleic sequence | number | sequence |

| | | | | |
|---|---|---|---|---|
| Full-length human | 1 | GCCCTGCCTGATGAGACAGAGGTGGTGGAAGAAACTGT | 2 | ALPDETEWEETVAEVTEVSVGANP |
| SPARC | | GGCAGAGGTGACTGAGGTATCTGTGGGAGCTAATCCTGT | | VQVEVGEFDDGAEETEEEVVAENP |
| | | CCAGGTGGAAGTAGGAGAATTTGATGATGGTGCAGAGGA | | CQNHHCKHGKVCELDENNTPMCVC |
| | | AACCGAAGAGGAGGTGGTGGCGGAAAATCCCTGCCAGA | | QDPTSCPAPIGEFEKVCSNDNKTF |
| | | ACCACCACTGCAAACACGGCAAGGTGTGCGAGCTGGAT | | DSSCHFFATKCTLEGTKKGHKLHL |
| | | GAGAACAACACCCCCATGTGCGTGTGCCAGGACCCCAC | | DYIGPCKYIPPCLDSELTEFPLRM |
| | | CAGCTGCCCAGCCCCCATTGGCGAGTTTGAGAAGGTGT | | RDWLKNVLVTLYERDEDNNLLTEK |
| | | GCAGCAATGACAACAAGACCTTCGACTCTTCCTGCCACT | | QKLRVKKIHENEKRLEAGDHPVEL |
| | | TCTTTGCCACAAAGTGCACCCTGGAGGGCACCAAGAAGG | | LARDFEKNYNMYIFPVHWQFGQLD |
| | | GCCACAAGCTCCACCTGGACTACATCGGGCCTTGCAAAT | | QHPIDGYLSHTELAPLRAPLIPME |
| | | ACATCCCCCCTTGCCTGGACTCTGAGCTGACCGATTCC | | HCTTRFFETCDLDNDKYIALDEWA |
| | | CCCTGCGCATGCGGGACTGGCTCAAGAACGTCCTGGTC | | GCFGIKQKDIDKDLVI |
| | | ACCCTGTATGAGAGGGATGAGGACAACAACCTTCTGACT | | |
| | | GAGAAGCAGAAGCTGCGGGTGAAGAAGATCCATGAGAAT | | |
| | | GAGAAGCGCCTGGAGGCAGGAGACCACCCCGTGGAGCT | | |
| | | GCTGGCCCGGGACTTCGAGAAGAACTATAACATGTACAT | | |
| | | CTTCCCTGTACACTGGCAGTTCGGCCAGCTGGACCAGCA | | |
| | | CCCCATTGACGGGTACCTCTCCCACACCGAGCTGGCTCC | | |
| | | ACTGCGTGCTCCCCTCATCCCCATGGAGCATTGCACCAC | | |
| | | CCGCTTTTTCGAGACCTGTGACCTGGACAATGACAAGTA | | |
| | | CATCGCCCTGGATGAGTGGGCCGGCTGCTTCGGCATCA | | |
| | | AGCAGAAGGATATCGACAAGGATCTTGTGATCTAA | | |
| 71v1 | 3 | ATGTCTGACAAACCCGATATGGCTGAGATCGAGAAATTC | 4 | MSDKPDMAEIEKFDKSKLKKTET |
| | | GATAAGTCGAAACTGAAGAAGACAGAGACGCAAGAGAAA | | QEKNPLPSKETIEQEKQAGES |
| | | AATCCACTGCCTTCCAAAGAAACGATTGAACAGGAGAAG | | |
| | | CAAGCAGGCGAATCGTAA | | |
| Full-length human | 5 | ATGATTATCTACCGGGACCTCATCAGCCACGATGAGATG | 6 | MIIYRDLISHDEMFSDIYKIREI |
| TPT1 | | TTCTCCGACATCTACAAGATCCGGGAGATCGCGGACGGG | | ADGLCLEVEGKMVSRTEGNIDDS |
| | | TTGTGCCTGGAGGTGGAGGGGAAGATGGTCAGTAGGAC | | LIGGNASAEGPEGEGTESTVITG |
| | | AGAAGGTAACATTGATGACTCGCTCATTGGTGGAAATGC | | VDIVMNHHLQETSFTKEAYKKYI |
| | | CTCCGCTGAAGGCCCCGAGGGCGAAGGTACCGAAAGCA | | KDYMKSIKGKLEEQRPERVKPFM |
| | | CAGTAATCACTGGTGTCGATATTGTCATGAACCATCACCT | | TGAAEQIKHILANFKNYQFFIGE |
| | | GCAGGAAACAAGTTTCACAAAAGAAGCCTACAAGAAGTA | | NMNPDGMVALLDYREDGVTPYMI |
| | | CATCAAAGATTACATGAAATCAATCAAAGGGAAACTTGAA | | FFKDGLEMEKC |
| | | GAACAGAGACCAGAAAGAGTAAAACCTTTTATGACAGGG | | |
| | | GCTGCAGAACAAATCAAGCACATCCTTGCTAATTTCAAAA | | |
| | | ACTACCAGTTCTTTATTGGTGAAAACATGAATCCAGATGG | | |
| | | CATGGTTGCTCTATTGGACTACCGTGAGGATGGTGTGAC | | |
| | | CCCATATATGATTTTCTTTAAGGATGGTTTAGAAATGGAA | | |
| | | AAATGTTAA | | |
| Full-length human | 7 | ATCCAGCGTACTCCAAAGATTCAGGTTTACTCACGTCATC | 8 | IQRTPKIQVYSRHPAENGKSNFL |
| B2M | | CAGCAGAGAATGGAAAGTCAAATTTCCTGAATTGCTATGT | | NCYVSGFHPSDIEVDLLKNGERI |
| | | GTCTGGGTTTCATCCATCCGACATTGAAGTTGACTTACTG | | EKVEHSDLSFSKDWSFYLLYYTE |
| | | AAGAATGGAGAGAGAATTGAAAAAGTGGAGCATTCAGAC | | FTPTEKDEYACRVNHVTLSQPKI |
| | | TTGTCTTTCAGCAAGGACTGGTCTTTCTATCTCTTGTACT | | VKWDRDM |
| | | ACACTGAATTCACCCCCACTGAAAAAGATGAGTATGCCT | | |
| | | GCCGTGTGAACCATGTGACTTTGTCACAGCCCAAGATAG | | |
| | | TTAAGTGGGATCGAGACATGTAA | | |
| Fragment human | 9 | GTGCCCATCCAAAAAGTCCAAGATGACACCAAAACCCTC | 10 | VPIQKVQDDTKTLIKTIVTRIND |
| LEP | | ATCAAGACAATTGTCACCAGGATCAATGACATTTCACACA | | ISHTQSVSSKQKVTGLDFIPGLH |
| | | CGCAGTCAGTCTCCTCCAAACAGAAAGTCACCGGTTTGG | | PILTLSKMDQTLAVYQQILTSMP |
| | | ACTTCATTCCTGGGCTCCACCCCATCCTGACCTTATCCAA | | SRNVIQISNDLENLRDLLHVLAF |
| | | GATGGACCAGACACTGGCAGTCTACCAACAGATCCTCAC | | SKSCHLPWASGLETLDSLGGVLE |
| | | CAGTATGCCTTCCAGAAACGTGATCCAAATATCCAACGAC | | ASGYSTEWALSRLQGSLQDMLWQ |
| | | CTGGAGAACCTCCGGGATCTTCTTCACGTGCTGGCCTTC | | LDLSPGC |
| | | TCTAAGAGCTGCCACTTGCCCTGGGCCAGTGGCCTGGA | | |
| | | GACCTTGGACAGCCTGGGGGGTGTCCTGGAAGCTTCAG | | |
| | | GCTACTCCACAGAGGTGGTGGCCCTGAGCAGGCTGCAG | | |
| | | GGGTCTCTGCAGGACATGCTGTGGCAGCTGGACCTCAG | | |
| | | CCCTGGGTGCTGA | | |
| Full-length human | 11 | ATGGCCAACATCGCGGTGCAGCGAATCAAGCGGGAGTT | 12 | MANIAVQRIKREFKEVLKSEETS |
| HP2 | | CAAGGAGGTGCTGAAGAGCGAGGAGACGAGCAAAAATC | | KNQIKVDLVDENFTELRGEIAGP |
| | | AAATTAAAGTAGATCTTGTAGATGAGAATTTTACAGAATTA | | PDTPYEGGRYQLEIKIPETYPFN |
| | | AGAGGAGAAATAGCAGGACCTCCAGACACACCATATGAA | | PPKVRFITKIWHPNISSVTGAIC |
| | | GGAGGMGATACCAACTAGAGATAAAAATACCAGAAACA | | LDILKDQWAAAMTLRTVLLSLQA |
| | | TACCCATTTAATCCCCCTAAGGTCCGGTTTATCACTAAAA | | LLAAAEPDDPQDAWANQYKQNPE |
| | | TATGGCATCCTAATATTAGTTCCGTCACAGGGGCTATTTG | | MFKQTARLWAHVYAGAPVSSPEY |
| | | TTTGGATATCCTGAAAGATCAATGGGCAGCTGCAATGACT | | TKKIENLCAMGFDRNAVIVALSS |
| | | CTCCGCACGGTATTATTGTCATTGCAAGCACTATTGGCAG | | KSWDVETATELLLSN |
| | | CTGCAGAGCCAGATGATCCACAGGATGCTGTAGTAGCA | | |
| | | ATCAGTACAAACAAAATCCCGAAATGTTCAAACAGACAGC | | |
| | | TCGACTTTGGGCACATGTGTATGCTGGAGCACCAGTTTC | | |
| | | TAGTCCAGAATACACCAAAAAAATAGAAAACCTATGTGCT | | |
| | | ATGGGCTTTGATAGGAATGCAGTAATAGTGGCCTTGTCTT | | |
| | | CAAAATCATGGGATGTAGAGACTGCAACAGAATTGCTTCT | | |
| | | GAGTAACTGA | | |
| Fragment human | 13 | GTCCTCCTGGAGGCCTGCTGTGCGGACGGACACCGGAT | 14 | VLLEACCADGHRMATHQKDCSLP |
| FBLN1 | | GGCCACTCATCAGAAGGACTGCTCGCTGCCATATGCTAC | | YATESKECRMVQEQCCHSQLEEL |
| | | GGAATCCAAAGAATGCAGGATGGTGCAGGAGCAGTGCT | | HCATGISLANEQDRCATPHGDNA |
| | | GCCACAGCCAGCTGGAGGAGCTGCACTGTGCCACGGGC | | SLEATFVKRCCHCCLLGRAAQAQ |
| | | ATCAGCCTGGCCAACGAGCAGGACCGCTGTGCCACGCC | | GQSCEYSLMVGYQCGQVFRACCV |
| | | CCACGGTGACAACGCCAGCCTGGAGGCCACATTTGTGM | | KSQETGDLDVGGLQETDKIIEVE |
| | | GAGGTGCTGCCATTGCTGTCTGCTGGGGAGGGCGGCCC | | EEQEDPYLNDRCRGGGPCKQQCR |
| | | AGGCCCAGGGCCAGAGCTGCGAGTACAGCCTCATGGTT | | DTGDEWCSCFVGYQLLSDGVSCE |
| | | GGCTACCAGTGTGGACAGGTCTTCCGGGCATGCTGTGTC | | DVNECITGSHSCRLGESCINTVG |
| | | AAGAGCCAGGAGACCGGAGATTTGGATGTCGGGGGCCT | | SFRCQRDSSCGTGYELTEDNSCK |
| | | CCAAGAAACGGATAAGATCATTGAGGTTGAGGAGGAACA | | DIDECESGHNCLPDFICQNTLGS |
| | | AGAGGACCCATATCTGAATGACCGCTGCCGAGGAGGCG | | FRCRPKLQCKSGFIQDALGNCID |
| | | GGCCCTGCAAGCAGCAGTGCCGAGACACGGGTGACGAG | | INECLSISAPCPIGHTCINTEGS |
| | | GTGGTCTGCTCCTGCTTCGTGGGCTACCAGCTGCTGTCT | | YTCQKNVPNCGRGYHLNEEGTRC |
| | | GATGGTGTCTCCTGTGAAGATGTCAATGAATGCATCACG | | VDVDECAPPAEPCGKGHRCVNSP |
| | | GGCAGCCACAGCTGCCGGCTTGGAGAATCCTGCATCAA | | GSFRCECKTGYYFDGISRMCVDV |
| | | CACAGTGGGCTCTTTCCGCTGCCAGCGGGACAGCAGCT | | NECQRYPGRLCGHKCENTLGSYL |
| | | GCGGGACTGGCTATGAGCTCACAGAGGACAATAGCTGC | | CSCSVGFRLSVDGRSCEDINECS |
| | | AAAGATATTGACGAGTGTGAGAGTGGTATTCATAACTGCC | | SSPCSQECANVYGSYQCYCRRGY |
| | | TCCCCGATTTTATCTGTCAGAATACTCTGGGATCCTTCCG | | QLSDVDGVTCEDIDECALPTGGH |
| | | CTGCCGACCCAAGCTACAGTGCAAGAGTGGCTTTATACA | | ICSYRGINIPGSFQCSCPSSGYR |
| | | AGATGCTCTAGGCAACTGTATTGATATCAATGAGTGTTTG | | LAPNGRNCGDIDECVTGIHNCSI |
| | | AGTATCAGTGCCCCGTGCCCTATTGGGCATACATGCATC | | NETCFNIQGAFRCLAFECPENYR |
| | | AACACAGAGGGCTCCTACACGTGCCAGAAGAACGTGCC | | RSAATRCERLPCHENRECSKLPL |
| | | CAACTGTGGCCGTGGCTACCATCTCAACGAGGAGGGAA | | RITYYHLSFPTNIQAPAWFRMGP |
| | | CGCGCTGTGTTGATGTGGACGAGTGCGCGCCACCTGCT | | SSAVPGDSMQLAITGGNEEGFFT |
| | | GAGCCCTGTGGGAAGGGACATC | | TRKVSPHSGWALTKPVPEPRDLL |
| | | | | LTVKMDLSRHGTVSSFVAKLFIF |
| | | | | VSAEL |
| Full-length human | 15 | ATGTACGAGCAACTCAAGGGCGAGTGGAACCGTAAAAGC | 16 | MYEQLKGEWNRKSPNLSKCGEEL |
| PSMD8 | | CCCAATCTTAGCAAGTGCGGGGAAGAGCTGGGTCGACT | | GRLKLVLLELNFLPTTGTKLTKQ |
| | | CAAGCTAGTTCTTCTGGAGCTCAACTTCTTGCCAACCACA | | QLILARDILEIGAQWSILRKDIP |
| | | GGGACCAAGCTGACCAAACAGCAGCTAATTCTGGCCCGT | | SFERYMAQLKCYYFDYKEQLPES |
| | | GACATACTGGAGATCGGGGCCCAATGGAGCATCCTACG | | AYMHQLLGLNLLFLLSQNRVAEF |
| | | CAAGGACATCCCCTCCTTCGAGCGCTACATGGCCCAGCT | | HTELERLPAKDIQTNVYIKHPVS |
| | | CAAATGCTACTACTTTGATTACAAGGAGCAGCTCCCCGA | | LEQYLMEGSYNKVFLAKGNIPAE |
| | | GTCAGCCTATATGCACCAGCTCTTGGGCCTCAACCTCCT | | SYTFFIDlLLDTIRDEIAGCIEK |
| | | CTTCCTGCTGTCCCAGAACCGGGTGGCTGAGTTCCACAC | | AYEKILFTEATRILFFNTPKKMT |
| | | GGAGTTGGAGCGGCTGCCTGCCAAGGACATACAGACCA | | DYAKKRGWVLGPNNYYSFASQQQ |
| | | ATGTCTACATCAAGCACCCAGTGTCCCTGGAGCAATACC | | KPEDTTIPSTELAKQVIEYARQL |
| | | TGATGGAGGGCAGCTACAACAAAGTGTTCCTGGCCAAGG | | EMIV |
| | | GTAACATCCCCGCCGAGAGCTACACCTTCTTCATTGACAT | | |
| | | CCTGCTCGACACTATCAGGGATGAGATCGCTGGGTGCAT | | |
| | | CGAGAAGGCCTACGAGAAAATCCTTTTCACTGAGGCCAC | | |
| | | CCGGATCCTCTTCTTCAACACACCCAAAAAGATGACAGA | | |
| | | CTACGCCAAGAAGCGAGGGTGGGTCCTGGGCCCCAACA | | |
| | | ACTACTACAGTTTTGCCAGCCAGCAGCAGAAGCCGGAAG | | |
| | | ACACCACCATTCCCTCCACAGAACTGGCCAAACAGGTCA | | |
| | | TCGAGTATGCCCGGCAGCTGGAGATGATCGTCTGA | | |
| Fragment human | 17 | ATGAGTCACACTGTCCAAACCTTCTTCTCTCCTGTCAACA | 18 | MSHTVQTFFSPVNSGQPPNYEML |
| IFITM3 | | GTGGCCAGCCCCCCAACTATGAGATGCTCAAGGAGGAG | | KEEHEVAVLGGPHNPAPPTSTVI |
| | | CACGAGGTGGCTGTGCTGGGGGGGCCCCACAACCCTGC | | HIRSETSVPDH |
| | | TCCCCCGACGTCCACCGTGATCCACATCCGCAGCGAGA | | |
| | | CCTCCGTGCCCGACCAT | | |
| Fragment human | 21 | ATGCGGCAGAAGGCGGTATCCGACGGACTAGTCATGCG | 22 | MRQKAVSLFLCYLLLFTCSGVEA |
| 1FI616 | | GCAGAAGGCGGTATCGCTTTTCTTGTGCTACCTGCTGCT | | GKKKCSESSDSGSGFWKA |
| | | CTTCACTTGCAGTGGGGTGGAGGCAGGTAAGAAAAAGTG | | |
| | | CTCGGAGAGCTCGGACAGCGGCTCCGGGTTCTGGAAGG | | |
| | | CT | | |
| Fragment human | 23 | ATGCGGTACGCCACCCACAAGTATCTCGATAGTGAGGAG | 24 | MRYATHKYLDSEEDEE |
| IFI616 | | GATGAGGAGTAG | | |
| Fragment human | 25 | ATGCGGCAGAAGGCGGTATCCGTTTTCTTGTGCTACCTG | 26 | MRQKAVSVFLCYLLLFTCSGVEA |
| IFI616 | | CTGCTCTTCACTTGCAGTGGGGTGGAGGCAGGTGAGAAT | | GENAGKDAGKKKCSESSDSGSGF |
| | | GCGGGTAAGGATGCAGGTAAGAAAAAGTGCTCGGAGAG | | WKALTFMAVGGGLAVAGLPALGF |
| | | CTCGGACAGCGGCTCCGGGTTCTGGAAGGCCCTGACCT | | TGAGIAANSVAASLMSWSAILNG |
| | | TCATGGCCGTCGGAGGAGGACTCGCAGTCGCCGGGCTG | | GGVPAGGLVATLQSLGAGGSSWI |
| | | CCCGCGCTGGGCTTCACCGGCGCCGGCATCGCGGCCAA | | GNIGALMRYATHKYLDSEEDEE |
| | | CTCGGTGGCTGCCTCGCTGATGAGCTGGTCTGCGATCCT | | |
| | | GAATGGGGGCGGCGTGCCCGCCGGGGGGCTAGTGGCC | | |
| | | ACGCTGCAGAGCCTCGGGGCTGGTGGCAGCAGCGTCGT | | |
| | | CATAGGTAATATTGGTGCCCTGATGCGGTACGCCACCCA | | |
| | | CAAGTATCTCGATAGTGAGGAGGATGAGGAGTAG | | |
| Human DVL1_v1 | 42 | ACCGACTCCACCATGTCCCTCAACATCGTCACTGTCACGCTC | 43 | TDSTMSLNIVTVTLNMERHHFLG |
| | | AACATGGAAGACATCACTTTCTGGGCATCAGCATCGTGGGGC | | ISIVGQSNDRGDGGIYIGSIMKG |
| | | AGAGCAACGACCGTGGAGACGGCGGCATCTACATTGGCTCCA | | GAVAADGRIEPGDMLLQVNDVNF |
| | | TCATGAAGGGCGGGGCTGTGGCCGCTGACGGCCGCATCGAGC | | ENMSNDDAVRVLREIVSQTGPIS |
| | | CCGGCGACATGTTGCTGCAGGTGAATGACGTGAACTTTGAGA | | LTVAKCWDPTPRSYFTVPRADPV |
| | | ACATGAGCAATCACGATGCCGTGCGGGTGCTGCGGGAGATCG | | RPIDPAAWLSHTAALTGALPRYE |
| | | TTTCCCAGACGGGGCCCATCAGCCTCACTGTGGCCAAGTGCT | | LE |
| | | GGGACCCAACGCCCCGAAGCTACTTCACCGTCCCACGGGCTG | | |
| | | ACCCGGTGCGGCCCATCGACCCCGCCGCCTGGCTGTCCCACA | | |
| | | CGGCGGCACTGACAGGAGCCCTGCCCCGCTACGAGCTGGAAG | | |
| Human DVL2_v1 | 44 | AGCCATGAGAATCTGGAGCCTGAGACAGAAACCGAGTCAGTA | 45 | SHENLEPHTETESVVSLRRERPR |
| | | GTGTCACTGAGGCGGGAGCGGCCTCGCAGGAGAGACAGCAGT | | RRDSSEHGAGGHRTGGPSRLHRH |
| | | GAGCATGGCGCTGGGGGCCACAGGACTGGTGGCCCCTCAAGG | | LAGYESSSTLMTSELESTSLGDS |
| | | CTGGAGCGCCACCTGGCCGGATACGAGAGCTCCTCTACCCTC | | DEHDTMSRFSSSTEQSSASRLLK |
| | | ATGACCAGCGAGCTGGAGAGTACCAGTCTGGGGGACTCGGAC | | RHRRRRKQRPPRLERTSSFSSVT |
| | | GAGGAGGACACCATGAGCAGGTTCAGCAGCTCCACGGAGCAG | | DSTMSLNIITVTLNMEKYNFLGI |
| | | AGCAGTGCCTCCCGCCTCCTTAAGCGCCACCGGCGGCGAAGG | | SIVGQSNERGDGGIYIGSIMKGG |
| | | AAGCAGAGGCCGCCCCGCCTGGAGAGGACGTCATCCTTCAGC | | AVAADGRIEPGDMLLQVNDMNFE |
| | | AGCGTCACAGATTCCACAATGTCTCTCAATATCATCACAGTC | | NMSNDDAVRVLRDIVHKPGPIVL |
| | | ACGCTAAACATGGAGAAGTACAACTTCCTGGGTATCTCCATT | | TVAKCWDPSPQAYFTLP |
| | | GTTGGCCAAAGCAATGAGCGGGGAGACGGAGGCATCTACATT | | |
| | | CGCTCCATCATGAAGGGTGGGGCTGTGGCGGCCGACGGGCGC | | |
| | | ATTGAGCCAGGGGACATGCTTTTGCAGGTGAATGACATGAAC | | |
| | | TTTGAGAACATGAGCAACGATGACGCTGTGCGGGTGCTGAGG | | |
| | | GACATTGTGCACAAGCCTGGCCCCATTGTGCTGACTGTGGCC | | |
| | | AAGTGCTGGGATCCCTCTCCTCAGGCCTATTTCACTCTCCCC | | |
| | | C | | |
| Human DVL3_v1 | 46 | CGGCGAGAACCAGGGGGTTATGATAGCTCATCCACCCTTATG | 47 | RREPGGYDSSSTLMSSELETTSF |
| | | AGCAGTGACCTGGAGACCACCAGCTTCTTTGACTCAGATGAG | | FDSDEDDSTSRFSSSTEQSSASR |
| | | GATGACTCCACCAGCAGGTTCAGCAGCTCCACAGAACAGAGC | | LMRRHKRRRRKQKVSRIERSSSF |
| | | AGTGCCTCACGCCTGATGAGAAGACACAAGCGGCGGCGGCGG | | SSITDSTMSLNIITVTLNMEKYN |
| | | AAGCAGAAGGTTTCTCGGATTGAGCGGTCCTCGTCCTTCAGC | | FLGISIVGQSNERGDGGIYIGSI |
| | | AGCATCACGGACTCCACCATGTCACTCAACATCATCACGGTC | | MKGGAVAADGRIEPGDMLLQVNE |
| | | ACTCTCAACATGGAAAATATAACTTCTTGGGCATCTCCATTG | | INFENMSNDDAVRVLREIVHKPG |
| | | TGGGCCAAAGCAACGAGCGTGGTGACGGCGGCATCTACATTG | | PITLTVAKCWDPSPRGCFTLPRS |
| | | GCTCTATCATGAAGGGTGGGGCCGTGGCTGCTGATGGACGCA | | EPIRPIDP |
| | | TCGAGCCAGGAGATATGTTGTTACAGGTAAACGAGATCAACT | | |
| | | TTGAGAACATGAGTAATGACGATGCAGTCCGGGTACTGCGGG | | |
| | | AGATTGTGCACAAACCGGGGCCCATCACCCTGACTGTAGCCA | | |
| | | AGTGCTGGGACCCAAGTCCACGTGGTTGCTTCACATTGCCCA | | |
| | | GGAGCGAGCCCATCCGGCCCATTGACCCT | | |

TABLE 2
bait-prey interactions

| | 2. | 3. | |
|---|---|---|---|
| | DNA | AA | |
| 1. | bait | bait | |
| Bait | SEQ | SEQ | |
| name | ID | ID | 4. Prey |
| Full-length | 1 | 2 | dbj\|AB033073.1\|AB033073 Homo sapiens mRNA for KIAA1247 protein, partial cds |
| human SPARC | | | |
| Full-length | 1 | 2 | dbj\|AB040964.1\|AB040964 Homo sapiens mRNA for KIAA1531 protein, partial cds |
| human SPARC | | | |
| Full-length | 1 | 2 | dbj\|AP001697.1\|AP001697 Homo sapiens genomic DNA, chromosome 21q, section 41/105 |
| human SPARC | | | |
| Full-length | 1 | 2 | emb\|AJ251973.1\|HSA251973 Homo sapiens partial steerin-1 gene |
| human SPARC | | | |
| Full-length | 1 | 2 | emb\|AL031666.5\|HS569M23 Human DNA sequence from clone 569M23 on chromosome 20q12-13.1 Contains 2 |
| human SPARC | | | isoforms of an mRNA for part of a gene similar to BS69, ESTs, CA repeat (D20S891), GSSs, complete |
| | | | sequence [Homo sapiens] |
| Full-length | 1 | 2 | emb\|AL035461.11\|HS967N21 Human DNA sequence from clone RP5-967N21 on chromosome 20p12.3-13. |
| human SPARC | | | Contains the CHGB gene for chromogranin B (secretogranin 1, SCG1), a pseudogene similar to part of |
| | | | KIAA0172, the gene for a novel protein and KIAA1153, the gene for a novel MCM2/3/5 fam |
| Full-length | 1 | 2 | emb\|AL049829.4\|CNS0000B Human chromosome 14 DNA sequence *** IN PROGRESS *** BAC R-124D2 of |
| human SPARC | | | library RPCI-11 from chromosome 14 of Homo sapiens (Human), complete sequence |
| Full-length | 1 | 2 | emb\|AL354977.10\|AL354977 Human DNA sequence from clone RP11-509J21 on chromosome 9, complete |
| human SPARC | | | sequence [Homo sapiens] |
| Full-length | 1 | 2 | emb\|AL391474.8\|AL391474 Human DNA sequence from clone RP13-115H14 on chromosome X Contains STSs, |
| human SPARC | | | GSSs and a CpG island, complete sequence [Homo sapiens] |
| Full-length | 1 | 2 | emb\|X15880.1\|HSCOL1C Human mRNA for collagen VI alpha-1 C-terminal globular domain |
| human SPARC | | | |
| Full-length | 1 | 2 | emb\|Z98036.1\|HS187N21 Human DNA sequence from PAC 187N21 on chromosome 6p21.2-6p21.33. Contains |
| human SPARC | | | ESTs |
| Full-length | 1 | 2 | gb\|AC002418.1\|AC002418 Human Chromosome X, complete sequence [Homo sapiens] |
| human SPARC | | | |
| Full-length | 1 | 2 | gb\|AC004890.2\|AC004890 Homo sapiens PAC clone RP4-800G7 from 7q35-q36, complete sequence |
| human SPARC | | | |
| Full-length | 1 | 2 | gb\|AC005038.5\|AC005038 Homo sapiens clone RP11-486I22, complete sequence |
| human SPARC | | | |
| Full-length | 1 | 2 | gb\|AC005399.19\|AC005399 Homo sapiens chromosome 22q11 clone p238m15, complete sequence |
| human SPARC | | | |
| Full-length | 1 | 2 | gb\|AC007000.2\|AC007000 Homo sapiens BAC clone RP11-467H10 from 7, complete sequence |
| human SPARC | | | |
| Full-length | 1 | 2 | gb\|AC007687.16\|AC007687 Homo sapiens 3q26.2-27 BAC RP11-494H4 (Roswell Park Cancer Institute Human |
| human SPARC | | | BAC Library) complete sequence |
| Full-length | 1 | 2 | gb\|AC010491.3\|AC010491 Homo sapiens chromosome 5 clone CTD-2335I23, complete sequence |
| human SPARC | | | |
| Full-length | 1 | 2 | gb\|AC022079.16\|AC022079 Homo sapiens 12 BAC RP11-425D17 (Roswell Park Cancer Institute Human BAC |
| human SPARC | | | Library) complete sequence |
| Full-length | 1 | 2 | gb\|AC024168.4\|AC024168 Homo sapiens chromosome 3 clone RP11-622P3 map 3p, complete sequence |
| human SPARC | | | |
| Full-length | 1 | 2 | gb\|AC078874.13\|AC078874 Homo sapiens 12q BAC RP11-781C15 (Roswell Park Cancer Institute Human BAC |
| human SPARC | | | Library) complete sequence |
| Full-length | 1 | 2 | gb\|AF023611.1\|AF023611 Homo sapiens Dim1p homolog (hdim1+) mRNA, complete cds |
| human SPARC | | | |
| Full-length | 1 | 2 | gb\|AF090190.1\|AF090190 Mus musculus kinesin-like protein KIF1B (Kif1b) mRNA, complete cds |
| human SPARC | | | |
| Full-length | 1 | 2 | gb\|AF090990.1\|AF090990 Homo sapiens high-risk human papilloma viruses E6 oncoproteins targeted protein |
| human SPARC | | | E6TP1 beta mRNA, complete cds |
| Full-length | 1 | 2 | gb\|BC000748.1\|BC000748 Homo sapiens, tubulin, beta, 4, clone MGC: 2411, mRNA, complete cds |
| human SPARC | | | |
| Full-length | 1 | 2 | gb\|BC004227.1\|BC004227 Homo sapiens, Similar to metastasis associated 1, clone MGC: 4778, mRNA, |
| human SPARC | | | complete cds |
| Full-length | 1 | 2 | gb\|J03866.1\|HUMIGMBC Homo sapiens M2 mitochondrial autoantigen dihydrolipoamide acetyltransferase |
| human SPARC | | | mRNA, complete cds |
| Full-length | 1 | 2 | gb\|L39210.1\|HUMIMPD Homo sapiens inosine monophosphate dehydrogenase type II gene, complete cds |
| human SPARC | | | |
| Full-length | 1 | 2 | gb\|L78833.1\|HUMBRCA1 Human BRCA1, Rho7 and vatl genes, complete cds, and ipf35 gene, partial cds |
| human SPARC | | | |
| Full-length | 1 | 2 | gb\|U33056.1\|HSU33056 Human G protein-coupled receptor kinase GRK4 mRNA, gamma splice variant, |
| human SPARC | | | complete cds |
| Full-length | 1 | 2 | ref\|NM_000918.1\| Homo sapiens procollagen-proline, 2-oxoglutarate 4-dioxygenase (proline 4-hydroxylase), |
| human SPARC | | | beta polypeptide (protein disulfide isomerase; thyroid hormone binding protein p55) (P4HB), mRNA |
| Full-length | 1 | 2 | ref\|NM_001159.2\| Homo sapiens aldehyde oxidase 1 (AOX1), mRNA |
| human SPARC | | | |
| Full-length | 1 | 2 | ref\|NM_001396.1\| Homo sapiens dual-specificity tyrosine-(Y)-phosphorylation regulated kinase 1 (DYRK1) |
| human SPARC | | | mRNA |
| Full-length | 1 | 2 | ref\|NM_001610.1\| Homo sapiens acid phosphatase 2, lysosomal (ACP2), mRNA |
| human SPARC | | | |
| Full-length | 1 | 2 | ref\|NM_002211.1\| Homo sapiens integrin, beta 1 (fibronectin receptor, beta polypeptide, antigen CD29 includes |
| human SPARC | | | MDF2, MSK12) (ITGB1), mRNA |
| Full-length | 1 | 2 | ref\|NM_002707.1\| Homo sapiens protein phosphatase 1G (formerly 2C), magnesium-dependent, gamma isoform |
| human SPARC | | | (PPM1G), mRNA |
| Full-length | 1 | 2 | ref\|NM_002719.1\| Homo sapiens protein phosphatase 2, regulatory subunit B (B56), gamma isoform (PPP2R5C), |
| human SPARC | | | mRNA |
| Full-length | 1 | 2 | ref\|NM_002836.1\| Homo sapiens protein tyrosine phosphatase, receptor type, A (PTPRA), mRNA |
| human SPARC | | | |
| Full-length | 1 | 2 | ref\|NM_003165.1\| Homo sapiens syntaxin binding protein 1 (STXBP1), mRNA |
| human SPARC | | | |
| Full-length | 1 | 2 | ref\|NM_003315.1\| Homo sapiens tetratricopeptide repeat domain 2 (TTC2), mRNA |
| human SPARC | | | |
| Full-length | 1 | 2 | ref\|NM_003321.1\| Homo sapiens Tu translation elongation factor, mitochondrial (TUFM), mRNA |
| human SPARC | | | |
| Full-length | 1 | 2 | ref\|NM_003373.2\| Homo sapiens vinculin (VCL), transcript variant VCL, mRNA |
| human SPARC | | | |
| Full-length | 1 | 2 | ref\|NM_003470.1\| Homo sapiens ubiquitin specific protease 7 (herpes virus-associated) (USP7), mRNA |
| human SPARC | | | |
| Full-length | 1 | 2 | ref\|NM_003752.2\| Homo sapiens eukaryotic translation initiation factor 3, subunit 8 (110 kD) (EIF3S8), mRNA |
| human SPARC | | | |
| Full-length | 1 | 2 | ref\|NM_003906.1\| Homo sapiens minichromosome maintenance deficient (S. cerevisiae ) 3-associated protein |
| human SPARC | | | (MCM3AP), mRNA |
| Full-length | 1 | 2 | ref\|NM_003940.1\| Homo sapiens ubiquitin specific protease 13 (isopeptidase T-3) (USP13), mRNA |
| human SPARC | | | |
| Full-length | 1 | 2 | ref\|NM_004227.2\| Homo sapiens pleckstrin homology, Sec7 and coiled/coil domains 3 (PSCD3), mRNA |
| human SPARC | | | |
| Full-length | 1 | 2 | ref\|NM_004369.1\| Homo sapiens collagen, type VI, alpha 3 (COL6A3), mRNA |
| human SPARC | | | |
| Full-length | 1 | 2 | ref\|NM 004568.1\| Homo sapiens serine (or cysteine) proteinase inhibitor, clade B (ovalbumin), member 6 |
| human SPARC | | | (SERPINB6), mRNA |
| Full-length | 1 | 2 | ref\|NM_004689.1\| Homo sapiens metastasis associated 1 (MTA1), mRNA |
| human SPARC | | | |
| Full-length | 1 | 2 | ref\|NM_004881.1\| Homo sapiens quinone oxidoreductase homolog (PIG3), mRNA |
| human SPARC | | | |
| Full-length | 1 | 2 | ref\|NM_005196.1\| Homo sapiens centromere protein F (350/400 kD, mitosin) (CENPF), mRNA |
| human SPARC | | | |
| Full-length | 1 | 2 | ref\|NM_005313.1\| Homo sapiens glucose regulated protein, 58 kD (GRP58), mRNA |
| human SPARC | | | |
| Full-length | 1 | 2 | ref\|NM_005507.1\| Homo sapiens cofilin 1 (non-muscle) (CFL1), mRNA |
| human SPARC | | | |
| Full-length | 1 | 2 | ref\|NM_005742.1\| Homo sapiens protein disulfide isomerase-related protein (P5), mRNA |
| human SPARC | | | |
| Full-length | 1 | 2 | ref\|NM_005744.2\| Homo sapiens ariadne (Drosophila) homolog, ubiquitin-conjugating enzyme E2-binding |
| human SPARC | | | protein, 1 (ARIH1), mRNA |
| Full-length | 1 | 2 | ref\|NM_005936.1\| Homo sapiens myeloid/lymphoid or mixed-lineage leukemia (trithorax (Drosophila) homolog); |
| human SPARC | | | translocated to, 4 (MLLT4), mRNA |
| Full-length | 1 | 2 | ref\|NM_005956.1\| Homo sapiens methylenetetrahydrofolate dehydrogenase (NADP+ ependent), |
| human SPARC | | | methenyltetrahydrofolate cyclohydrolase, formyltetrahydrofolate synthetase (MTHFD1), mRNA |
| Full-length | 1 | 2 | ref\|NM_006012.1\| Homo sapiens ClpP (caseinolytic protease, ATP-dependent, proteolytic subunit, E. coli ) |
| human SPARC | | | homolog (CLPP), nuclear gene encoding mitochondrial protein, mRNA |
| Full-length | 1 | 2 | ref\|NM_006373.1\| Homo sapiens membrane protein of cholinergic synaptic vesicles (VATI), mRNA |
| human SPARC | | | |
| Full-length | 1 | 2 | ref\|NM_006521.2\| Homo sapiens transcription factor binding to IGHM enhancer 3 (TFE3), mRNA |
| human SPARC | | | |
| Full-length | 1 | 2 | ref\|NM_006688.1\| Homo sapiens C1q-related factor (CRF), mRNA |
| human SPARC | | | |
| Full-length | 1 | 2 | ref\|NM_007214.1\| Homo sapiens SEC63, endoplasmic reticulum translocon component (S. cerevisiae ) like |
| human SPARC | | | (SEC63L), mRNA |
| Full-length | 1 | 2 | ref\|NM_014314.1\| Homo sapiens RNA helicase (RIG-I), mRNA |
| human SPARC | | | |
| Full-length | 1 | 2 | ref\|NM_014710.1\| Homo sapiens KIAA0443 gene product (KIAA0443), mRNA |
| human SPARC | | | |
| Full-length | 1 | 2 | ref\|NM_015456.1\| Homo sapiens DKFZP586B0519 protein (DKFZP586B0519), mRNA |
| human SPARC | | | |
| Full-length | 1 | 2 | ref\|NM_016174.1\| Homo sapiens cerebral cell adhesion molecule (LOC51148), mRNA |
| human SPARC | | | |
| Full-length | 1 | 2 | ref\|NM_017456.1\| Homo sapiens pleckstrin homology, Sec7 and coiled/coil domains 1(cytohesin 1) (PSCD1), |
| human SPARC | | | transcript variant 2, mRNA |
| Full-length | 1 | 2 | ref\|NM_017775.1\| Homo sapiens hypothetical protein FLJ20343 (FLJ20343), mRNA |
| human SPARC | | | |
| Full-length | 1 | 2 | ref\|NM_018046.1\| Homo sapiens hypothetical protein FLJ10283 (FLJ10283), mRNA |
| human SPARC | | | |
| Full-length | 1 | 2 | ref\|NM_019070.1\| Homo sapiens hypothetical protein (FLJ10432), mRNA |
| human SPARC | | | |
| Full-length | 1 | 2 | ref\|NM_021976.1\| Homo sapiens retinoid X receptor, beta (RXRB), mRNA |
| human SPARC | | | |
| Full-length | 1 | 2 | ref\|NM_024408.1\| Homo sapiens Notch (Drosophila) homolog 2 (NOTCH2), mRNA |
| human SPARC | | | |
| Full-length | 1 | 2 | ref\|NM_024636.1\| Homo sapiens hypothetical protein FLJ23153 (FLJ23153), mRNA |
| human SPARC | | | |
| Full-length | 5 | 6 | ref\|NM_001960.1\| Homo sapiens eukaryotic translation elongation factor 1 delta (guanine nucleotide exchange |
| human TPT1 | | | protein) (EEF1D), mRNA |
| Full-length | 5 | 6 | ref\|NM_002810.1\| Homo sapiens proteasome (prosome, macropain) 26S subunit, non-ATPase, 4 (PSMD4), |
| human TPT2 | | | mRNA |
| Full-length | 5 | 6 | ref\|NM_018249.1\| Homo sapiens hypothetical protein FLJ10867 (FLJ10867), mRNA |
| human TPT3 | | | |
| Full-length | 7 | 8 | dbj\|AP000130.1\|AP000130 Homo sapiens genomic DNA of 21q22.1, GART and AML, f43D11-119B8 region, |
| human B2M | | | segment 5/10, complete sequence |
| Full-length | 7 | 8 | emb\|AL445647.6\|AL445647 Human DNA sequence from clone RP11-471M2 on chromosome 13, complete |
| human B2M | | | sequence [Homo sapiens] |
| Full-length | 7 | 8 | gb\|AC009949.9\|AC009949 Homo sapiens clone RP11-69J7, complete sequence |
| human B2M | | | |
| Full-length | 7 | 8 | gb\|AF075587.1\|AF075587 Homo sapiens protein associated with Myc mRNA, complete cds |
| human B2M | | | |
| Full-length | 7 | 8 | ref\|NM_001273.1\| Homo sapiens chromodomain helicase DNA binding protein 4 (CHD4), mRNA |
| human B2M | | | |
| Full-length | 7 | 8 | ref\|NM_001469.1\| Homo sapiens thyroid autoantigen 70 kD (Ku antigen) (G22P1), mRNA |
| human B2M | | | |
| Full-length | 7 | 8 | ref\|NM_001813.1\| Homo sapiens centromere protein E (312 kD) (CENPE), mRNA |
| human B2M | | | |
| Full-length | 7 | 8 | ref\|NM_001998.1\| Homo sapiens fibulin 2 (FBLN2), mRNA |
| human B2M | | | |
| Full-length | 7 | 8 | ref\|NM_002567.1\| Homo sapiens prostatic binding protein (PBP), mRNA |
| human B2M | | | |
| Full-length | 7 | 8 | ref\|NM_002631.1\| Homo sapiens phosphogluconate dehydrogenase (PGD), mRNA |
| human B2M | | | |
| Full-length | 7 | 8 | ref\|NM_004628.1\| Homo sapiens xeroderma pigmentosum, complementation group C (XPC), mRNA |
| human B2M | | | |
| Full-length | 7 | 8 | ref\|NM_018950.1\| Homo sapiens major histocompatibility complex, class I, F (HLA-F), mRNA |
| human B2M | | | |
| Fragment | 9 | 10 | dbj\|AB002318.1\|AB002318 Human mRNA for KIAA0320 gene, partial cds |
| human LEP | | | |
| Fragment | 9 | 10 | dbj\|AB018325.1\|AB018325 Homo sapiens mRNA for KIAA0782 protein, partial cds |
| human LEP | | | |
| Fragment | 9 | 10 | dbj\|AB040964.1\|AB040964 Homo sapiens mRNA for KIAA1531 protein, partial cds |
| human LEP | | | |
| Fragment | 9 | 10 | dbj\|AK001362.1\|AK001362 Homo sapiens cDNA FLJ10500 fis, clone NT2RP2000369 |
| human LEP | | | |
| Fragment | 9 | 10 | dbj\|AK001362.1\|AK001362 Homo sapiens cDNA FLJ10500 fis, clone NT2RP2000369 |
| human LEP | | | |
| Fragment | 9 | 10 | dbj\|AK021423.1\|AK021423 Homo sapiens cDNA FLJ11361 fis, clone HEMBA1000243 |
| human LEP | | | |
| Fragment | 9 | 10 | dbj\|AK026882.1\|AK026882 Homo sapiens cDNA: FLJ23229 fis, clone CAE06852 |
| human LEP | | | |
| Fragment | 9 | 10 | dbj\|D83782.1\|D83782 Human mRNA for KIAA0199 gene, partial cds |
| human LEP | | | |
| Fragment | 9 | 10 | emb\|AJ251833.1\|HSA251833 Homo sapiens mRNA for hypothetical protein (ORF1 DNA) |
| human LEP | | | |
| Fragment | 9 | 10 | emb\|AL359619.1\|HSM802739 Homo sapiens mRNA; cDNA DKFZp762L0311 (from clone DKFZp762L0311) |
| human LEP | | | |
| Fragment | 9 | 10 | emb\|Z84487.2\|HS346O6 Human DNA sequence *** SEQUENCING IN PROGRESS *** from clone 346O6, |
| human LEP | | | complete sequence [Homo sapiens] |
| Fragment | 9 | 10 | gb\|AC004139.1\|AC004139 Homo sapiens chromosome 17, clone HRPC843B9, complete sequence |
| human LEP | | | |
| Fragment | 9 | 10 | gb\|AC005343.1\|AC005343 Homo sapiens Chromosome 12p13.3 BAC RPCI11-21K20 (Roswell Park Cancer |
| human LEP | | | Institute Human BAC Library) complete sequence |
| Fragment | 9 | 10 | gb\|AC007619.22\|AC007619 Homo sapiens 12p BAC RP11-253I19 (Roswell Park Cancer Institute Human BAC |
| human LEP | | | Library) complete sequence |
| Fragment | 9 | 10 | gb\|AC007999.11\|AC007999 Homo sapiens 3q25-26 BAC CTB-177N7 (California Institute of Technology BAC |
| human LEP | | | Library) complete sequence |
| Fragment | 9 | 10 | gb\|AC010450.6\|AC010450 Homo sapiens chromosome 5 clone CTD-2242D16, complete sequence |
| human LEP | | | |
| Fragment | 9 | 10 | gb\|AF098482.1\|AF098482 Homo sapiens transcriptional coactivator p52 mRNA, complete cds |
| human LEP | | | |
| Fragment | 9 | 10 | gb\|AF104313.1\|AF104313 Homo sapiens unknown mRNA |
| human LEP | | | |
| Fragment | 9 | 10 | gb\|AF139184.1\|AF139184 Homo sapiens Sec31 protein mRNA, complete cds |
| human LEP | | | |
| Fragment | 9 | 10 | gb\|BC002931.1\|BC002931 Homo sapiens, clone MGC: 11369, mRNA, complete cds |
| human LEP | | | |
| Fragment | 9 | 10 | gb\|M64497.1\|HUMARP1 Human apolipoprotein AI regulatory protein (ARP-1) mRNA, complete cds |
| human LEP | | | |
| Fragment | 9 | 10 | ref\|NM_000230.1\| Homo sapiens leptin (murine obesity homolog) (LEP), mRNA |
| human LEP | | | |
| Fragment | 9 | 10 | ref\|NM_001903.1\| Homo sapiens catenin (cadherin-associated protein), alpha 1 (102 kD) (CTNNA1), mRNA |
| human LEP | | | |
| Fragment | 9 | 10 | ref\|NM_002807.1\| Homo sapiens proteasome (prosome, macropain) 26S subunit, non-ATPase, 1 (PSMD1), |
| human LEP | | | mRNA |
| Fragment | 9 | 10 | ref\|NM_003024.1\| Homo sapiens intersectin 1 (SH3 domain protein) (ITSN1), mRNA |
| human LEP | | | |
| Fragment | 9 | 10 | ref\|NM_003127.1\| Homo sapiens spectrin, alpha, non-erythrocytic 1 (alpha-fodrin) (SPTAN1), mRNA |
| human LEP | | | |
| Fragment | 9 | 10 | ref\|NM_003404.1\| Homo sapiens tyrosine 3-monooxygenase/tryptophan 5-monooxygenase activation protein, |
| human LEP | | | beta polypeptide (YWHAB), mRNA |
| Fragment | 9 | 10 | ref\|NM_003981.1\| Homo sapiens protein regulator of cytokinesis 1 (PRC1), mRNA |
| human LEP | | | |
| Fragment | 9 | 10 | ref\|NM_004587.1\| Homo sapiens ribosome binding protein 1 (dog 180 kD homolog) (RRBP1), mRNA |
| human LEP | | | |
| Fragment | 9 | 10 | ref\|NM_004651.1\| Homo sapiens ubiquitin specific protease 11 (USP11), mRNA |
| human LEP | | | |
| Fragment | 9 | 10 | ref\|NM_004656.1\| Homo sapiens BRCA1 associated protein-1 (ubiquitin carboxy-terminal hydrolase) (BAP1), |
| human LEP | | | mRNA |
| Fragment | 9 | 10 | ref\|NM_004913.1\| Homo sapiens chromosome 16 open reading frame 7 (C16ORF7), mRNA |
| human LEP | | | |
| Fragment | 9 | 10 | ref\|NM_004945.1\| Homo sapiens dynamin 2 (DNM2), mRNA |
| human LEP | | | |
| Fragment | 9 | 10 | ref\|NM_005898.1\| Homo sapiens membrane component, chromosome 11, surface marker 1 (M11S1), mRNA |
| human LEP | | | |
| Fragment | 9 | 10 | ref\|NM_006006.1\| Homo sapiens zinc finger protein 145 (Kruppel-like, expressed in promyelocytic leukemia) |
| human LEP | | | (ZNF145), mRNA |
| Fragment | 9 | 10 | ref\|NM_006470.1\| Homo sapiens estrogen-responsive B box protein (EBBP), mRNA |
| human LEP | | | |
| Fragment | 9 | 10 | ref\|NM_006534.1\| Homo sapiens nuclear receptor coactivator 3 (NCOA3), mRNA |
| human LEP | | | |
| Fragment | 9 | 10 | ref\|NM_012075.1\| Homo sapiens Conserved gene telomeric to alpha globin cluster (CGTHBA), mRNA |
| human LEP | | | |
| Fragment | 9 | 10 | ref\|NM_014350.1\| Homo sapiens TNF-induced protein (GG2-1), mRNA |
| human LEP | | | |
| Fragment | 9 | 10 | ref\|NM_014670.1\| Homo sapiens KIAA0005 gene product (KIAA0005), mRNA |
| human LEP | | | |
| Fragment | 9 | 10 | ref\|NM_014786.1\| Homo sapiens KIAA0337 gene product (KIAA0337), mRNA |
| human LEP | | | |
| Fragment | 9 | 10 | ref\|NM_014865.1\| Homo sapiens chromosome condensation-related SMC-associated protein 1 (KIAA0159), |
| human LEP | | | mRNA |
| Fragment | 9 | 10 | ref\|NM_015456.1\| Homo sapiens DKFZP586B0519 protein (DKFZP586B0519), mRNA |
| human LEP | | | |
| Fragment | 9 | 10 | ref\|NM_015658.1\| Homo sapiens DKFZP564C186 protein (DKFZP564C186), mRNA |
| human LEP | | | |
| Fragment | 9 | 10 | ref\|NM_016221.1\| Homo sapiens dynactin p62 subunit (LOC51164), mRNA |
| human LEP | | | |
| Fragment | 9 | 10 | ref\|NM_016238.1\| Homo sapiens anaphase-promoting complex subunit 7 (APC7), mRNA |
| human LEP | | | |
| Fragment | 9 | 10 | ref\|NM_022340.1\| Homo sapiens FYVE-finger-containing Rab5 effector protein rabenosyn-5 (LOC64145), |
| human LEP | | | mRNA |
| Human HIP2 | 11 | 12 | dbj\|AB007880.1\|AB007880 Homo sapiens KIAA0420 mRNA, complete cds |
| (full-length) | | | |
| Human HIP2 | 11 | 12 | dbj\|AB011116.1\|AB011116 Homo sapiens mRNA for KIAA0544 protein, partial cds |
| (full-length) | | | |
| Human HIP2 | 11 | 12 | dbj\|AB023182.1\|AB023182 Homo sapiens mRNA for KIAA0965 protein, partial cds |
| (full-length) | | | |
| Human HIP2 | 11 | 12 | dbj\|AB025194.1\|AB025194 Homo sapiens mRNA for protein tyrosine phosphatase HD-PTP, complete cds |
| (full-length) | | | |
| Human HIP2 | 11 | 12 | dbj\|AB036063.1\|AB036063 Homo sapiens p53R2 mRNA for ribonucleotide reductase, complete cds |
| (full-length) | | | |
| Human HIP2 | 11 | 12 | dbj\|AB037744.1\|AB037744 Homo sapiens mRNA for KIAA1323 protein, partial cds |
| (full-length) | | | |
| Human HIP2 | 11 | 12 | dbj\|AK001025.1\|AK001025 Homo sapiens cDNA FLJ10163 fis, clone HEMBA1003568, weakly similar |
| (full-length) | | | to 52 KD RO PROTEIN |
| Human HIP2 | 11 | 12 | emb\|AJ007398.1\|HSA7398 Homo sapiens mRNA for PBK1 protein |
| (full-length) | | | |
| Human HIP2 | 11 | 12 | emb\|AL391221.15\|AL391221 Human DNA sequence from clone RP11-771D21 on chromosome 6, complete |
| (full-length) | | | sequence [Homo sapiens] |
| Human HIP2 | 11 | 12 | emb\|AL442167.1\|HSMX1B Homo sapiens chromosome 21 from 5 PACs and 5 Cosmids map 21q22.2, D21S349- |
| (full-length) | | | MX1; segment 2/2, complete sequence |
| Human HIP2 | 11 | 12 | emb\|Z46606.1\|HSHLTF1 H. sapiens HLTF gene for helicase-like transcription factor |
| (full-length) | | | |
| Human HIP2 | 11 | 12 | emb\|Z75889.1\|HS267P19 Human DNA sequence from cosmid 267P19, BRCA2 gene region chromosome |
| (full-length) | | | 13q12-13 contains polymorphic CA repeat |
| Human HIP2 | 11 | 12 | gb\|AC002073.1\|AC002073 Human PAC clone RP3-515N1 from 22q11.2-q22, complete sequence [Homo |
| (full-length) | | | sapiens] |
| Human HIP2 | 11 | 12 | gb\|AC005185.1\|AC005185 Homo sapiens Xp22 bins 169-171 BAC GSHB-383H3 (Genome Systems |
| (full-length) | | | Human BAC Library) complete sequence |
| Human HIP2 | 11 | 12 | gb\|AC006101.3\|AC006101 citb_338_f_24, complete sequence [Homo sapiens] |
| (full-length) | | | |
| Human HIP2 | 11 | 12 | gb\|AC007245.3\|AC007245 Homo sapiens BAC clone RP11-273L18 from 7, complete sequence |
| (full-length) | | | |
| Human HIP2 | 11 | 12 | gb\|AC007999.11\|AC007999 Homo sapiens 3q25-26 BAC CTB-177N7 (California Institute of Technology BAC |
| (full-length) | | | Library) complete sequence |
| Human HIP2 | 11 | 12 | gb\|AC015801.25\|AC015801 Homo sapiens chromosome 17, clone RP11-854A13, complete sequence |
| (full-length) | | | |
| Human HIP2 | 11 | 12 | gb\|AF083127.1\|AF083127 Homo sapiens CATX-11 mRNA, partial cds |
| (full-length) | | | |
| Human HIP2 | 11 | 12 | gb\|AF161453.1\|AF161453 Homo sapiens HSPC335 mRNA, partial cds |
| (full-length) | | | |
| Human HIP2 | 11 | 12 | gb\|AF180425.2\|AF180425 Homo sapiens retinoblastoma-associated protein RAP140 mRNA, complete cds |
| (full-length) | | | |
| Human HIP2 | 11 | 12 | gb\|AF307339.1\|AF307339 Homo sapiens B aggressive lymphoma short isoform (BAL) mRNA, omplete cds |
| (full-length) | | | |
| Human HIP2 | 11 | 12 | ref\|NM_000304.1\| Homo sapiens peripheral myelin protein 22 (PMP22), mRNA |
| (full-length) | | | |
| Human HIP2 | 11 | 12 | ref\|NM_001034.1\| Homo sapiens ribonucleotide reductase M2 polypeptide (RRM2), mRNA |
| (full-length) | | | |
| Human HIP2 | 11 | 12 | ref\|NM_001282.1\| Homo sapiens adaptor-related protein complex 2, beta 1 subunit (AP2B1), mRNA |
| (full-length) | | | |
| Human HIP2 | 11 | 12 | ref\|NM_001634.3\| Homo sapiens S-adenosylmethionine decarboxylase 1 (AMD1), mRNA |
| (full-length) | | | |
| Human HIP2 | 11 | 12 | ref\|NM_002504.1\| Homo sapiens nuclear transcription factor, X-box binding 1 (NFX1), mRNA |
| (full-length) | | | |
| Human HIP2 | 11 | 12 | ref\|NM_002508.1\| Homo sapiens nidogen (enactin) (NID), mRNA |
| (full-length) | | | |
| Human HIP2 | 11 | 12 | ref\|NM_002815.1\| Homo sapiens proteasome (prosome, macropain) 26S subunit, non-ATPase, 11 (PSMD11), |
| (full-length) | | | mRNA |
| Human HIP2 | 11 | 12 | ref\|NM_002931.2\| Homo sapiens ring finger protein 1 (RING1), mRNA |
| (full-length) | | | |
| Human HIP2 | 11 | 12 | ref\|NM_002959.2\| Homo sapiens sortilin 1 (SORT1), mRNA |
| (full-length) | | | |
| Human HIP2 | 11 | 12 | ref\|NM_003128.1\| Homo sapiens spectrin, beta, non-erythrocytic 1 (SPTBN1), mRNA |
| (full-length) | | | |
| Human HIP2 | 11 | 12 | ref\|NM_003664.1\| Homo sapiens adaptor-related protein complex 3, beta 1 subunit (AP3B1), mRNA |
| (full-length) | | | |
| Human HIP2 | 11 | 12 | ref\|NM_004749.1\| Homo sapiens cell cycle progression 2 protein (CPR2), mRNA |
| (full-length) | | | |
| Human HIP2 | 11 | 12 | ref\|NM_006913.1\| Homo sapiens ring finger protein 5 (RNF5), mRNA |
| (full-length) | | | |
| Human HIP2 | 11 | 12 | ref\|NM_014764.1\| Homo sapiens DAZ associated protein 2 (DAZAP2), mRNA |
| (full-length) | | | |
| Human HIP2 | 11 | 12 | ref\|NM_016271 .1\| Homo sapiens STRIN protein (STRIN), mRNA |
| (full-length) | | | |
| Human HIP2 | 11 | 12 | ref\|NM_016586.1\| Homo sapiens MBIP protein (MBIP), mRNA |
| (full-length) | | | |
| Human HIP2 | 11 | 12 | ref\|NM_017958.1\| Homo sapiens hypothetical protein FLJ20783 (FLJ20783), mRNA |
| (full-length) | | | |
| Human HIP2 | 11 | 12 | ref\|NM_018683.1\| Homo sapiens zinc finger protein 313 (ZNF313), mRNA |
| (full-length) | | | |
| Human HIP2 | 11 | 12 | ref\|NM_021253.1\| Homo sapiens ring finger protein 23 (RNF23), mRNA |
| (full-length) | | | |
| Human HIP2 | 11 | 12 | ref\|NM_022826.1\| Homo sapiens hypothetical protein DKFZp586F1122 similar to axotrophin |
| (full-length) | | | (DKFZP586F1122), mRNA |
| Human FBLN1 | 13 | 14 | dbj\|AB012692.1\|AB012692 Homo sapiens CAC-1 mRNA, partial cds |
| (fragment) | | | |
| Human FBLN1 | 13 | 14 | dbj\|AB037742.1\|AB037742 Homo sapiens mRNA for KIAA1321 protein, partial cds |
| (fragment) | | | |
| Human FBLN1 | 13 | 14 | dbj\|AK024191.1\|AK024191 Homo sapiens cDNA FLJ14129 fis, clone MAMMA1002617, weakly similar |
| (fragment) | | | to ZINC FINGER PROTEIN 135 |
| Human FBLN1 | 13 | 14 | dbj\|AP001753.1\|AP001753 Homo sapiens genomic DNA, chromosome 21q, section 97/105 |
| (fragment) | | | |
| Human FBLN1 | 13 | 14 | emb\|AJ006997.1\|HS190A9 Homo sapiens chromosome 21 PAC RPCIP704A9190Q2, complete sequence |
| (fragment) | | | |
| Human FBLN1 | 13 | 14 | emb\|AL121905.23\|HSDJ534B8 Human DNA sequence from clone RP4-534B8 on chromosome 20 Contains |
| (fragment) | | | ESTs, STSs, GSSs and CpG islands. Contains the PTPRA gene for two isoforms of the alpha polypeptide |
| | | | of receptor type protein tyrosine phosphatase, the GNRH2 gene for gonadotropin-releasi |
| Human FBLN1 | 13 | 14 | emb\|AL390878.6\|AL390878 Human DNA sequence from clone RP11-442F12 on chromosome 13, complete |
| (fragment) | | | sequence [Homo sapiens] |
| Human FBLN1 | 13 | 14 | emb\|Z69084.1\|HS361H4A Human DNA sequence from cosmid 361H4A, Huntington's Disease Region, |
| (fragment) | | | chromosome 4p16.3 |
| Human FBLN1 | 13 | 14 | emb\|Z99916.1\|HS221G9 Human DNA sequence from clone CTA-221G9 on chromosome 22q11.21-12.2 |
| (fragment) | | | Contains the CRYBB3 and CRYBB2 genes for Beta Crystallin B3 and B2, and a Stathmin family (SCG10-like, |
| | | | SCLIP, Neuroplasticin-2, NCP2) pseudogene, ESTs and GSSs, complete sequence [Homo |
| Human FBLN1 | 13 | 14 | gb\|AC006343.2\|AC006343 Homo sapiens PAC clone RP4-548K24 from 7q34-q36, complete sequence |
| (fragment) | | | |
| Human FBLN1 | 13 | 14 | gb\|AC008745.6\|AC008745 Homo sapiens chromosome 19 clone CTD-2571L23, complete sequence |
| (fragment) | | | |
| Human FBLN1 | 13 | 14 | gb\|AC012153.10\|AC012153 Homo sapiens 11q BAC RP5-1045O21 (Roswell Park Cancer Institute Human BAC |
| (fragment) | | | Library) complete sequence |
| Human FBLN1 | 13 | 14 | gb\|AC020728.4\|AC020728 Homo sapiens BAC clone RP11-506H20 from 5, complete sequence |
| (fragment) | | | |
| Human FBLN1 | 13 | 14 | gb\|AC022150.5\|AC022150 Homo sapiens chromosome 19 clone CTD-3099C6, complete sequence |
| (fragment) | | | |
| Human FBLN1 | 13 | 14 | gb\|AC023512.28\|AC023512 Homo sapiens 12 BAC RP11-1018J11 (Roswell Park Cancer Institute Human BAC |
| (fragment) | | | Library) complete sequence |
| Human FBLN1 | 13 | 14 | gb\|AC026187.3\|AC026187 Homo sapiens chromosome 3 clone RP11-306H5 map 3p, complete sequence |
| (fragment) | | | |
| Human FBLN1 | 13 | 14 | gb\|AC079030.13\|AC079030 Homo sapiens 12 BAC RP3-342K15 (Roswell Park Cancer Institute Human BAC |
| (fragment) | | | Library) complete sequence |
| Human FBLN1 | 13 | 14 | gb\|AF016903.1\|AF016903 Homo sapiens agrin precursor mRNA, partial cds |
| (fragment) | | | |
| Human FBLN1 | 13 | 14 | gb\|AF108830.1\|AF108830 Homo sapiens serine-threonine protein kinase (MNBH) mRNA, complete cds |
| (fragment) | | | |
| Human FBLN1 | 13 | 14 | gb\|AF131216.1\|AF131216 Homo sapiens chromosome 8 map 8p23-p22 clones CTB-164D9, CTB-169o5, CTB- |
| (fragment) | | | 65D4, CTC-271o23, CTC-367J24, complete sequence; and clones CTC-493P15, CTB-91N18, partial sequence |
| Human FBLN1 | 13 | 14 | gb\|AF159141.1\|AF159141 Homo sapiens breast cancer metastasis-suppressor 1 (BRMS1) mRNA, complete cds |
| (fragment) | | | |
| Human FBLN1 | 13 | 14 | gb\|AF165926.2\|AF165926 Homo sapiens chromosome 5p13 BAC clone djn085o06 containing NUP155 gene, |
| (fragment) | | | complete sequence |
| Human FBLN1 | 13 | 14 | gb\|AF183423.1\|AF183423 Homo sapiens reticulocabin precursor mRNA, complete cds |
| (fragment) | | | |
| Human FBLN1 | 13 | 14 | gb\|AF308301.1\|AF308301 Homo sapiens serologically defined breast cancer antigen NY-BR-87 mRNA, |
| (fragment) | | | partial cds |
| Human FBLN1 | 13 | 14 | gb\|AF330040.1\|AF330040 Homo sapiens IL-5 promoter REII-region-binding protein (REIIBP) mRNA, complete |
| (fragment) | | | cds |
| Human FBLN1 | 13 | 14 | gb\|BC003526.1\|BC003526 Homo sapiens, clone IMAGE: 3607457, mRNA, partial cds |
| (fragment) | | | |
| Human FBLN1 | 13 | 14 | ref\|NM_000358.1\| Homo sapiens transforming growth factor, beta-induced, 68 kD (TGFBI), mRNA |
| (fragment) | | | |
| Human FBLN1 | 13 | 14 | ref\|NM_001396.1\| Homo sapiens dual-specificity tyrosine-(Y)-phosphorylation regulated kinase |
| (fragment) | | | 1 (DYRK1) mRNA |
| Human FBLN1 | 13 | 14 | ref\|NM_001424.1\| Homo sapiens epithelial membrane protein 2 (EMP2), mRNA |
| (fragment) | | | |
| Human FBLN1 | 13 | 14 | ref\|NM_001694.1\| Homo sapiens ATPase, H+ transporting, lysosomal (vacuolar proton pump) 16 kD (ATP6L), |
| (fragment) | | | mRNA |
| Human FBLN1 | 13 | 14 | ref\|NM_003429.1\| Homo sapiens zinc finger protein 85 (HPF4, HTF1) (ZNF85), mRNA |
| (fragment) | | | |
| Human FBLN1 | 13 | 14 | ref\|NM_006598.1\| Homo sapiens solute carrier family 12 (potassium/chloride transporters), |
| (fragment) | | | member 7 (SLC12A7), mRNA |
| Human FBLN1 | 13 | 14 | ref\|NM_006663.1\| Homo sapiens ReIA-associated inhibitor (RAI), mRNA |
| (fragment) | | | |
| Human FBLN1 | 13 | 14 | ref\|NM_007456.1\| Mus musculus adaptor-related protein complex AP-1, mu subunit 1 (Ap1m1), mRNA |
| (fragment) | | | |
| Human FBLN1 | 13 | 14 | ref\|NM_012215.1\| Homo sapiens meningioma expressed antigen 5 (hyaluronidase) (MGEA5), mRNA |
| (fragment) | | | |
| Human FBLN1 | 13 | 14 | ref\|NM_014745.1\| Homo sapiens KIAA0233 gene product (KIAA0233), mRNA |
| (fragment) | | | |
| Human FBLN1 | 13 | 14 | ref\|NM_014772.1\| Homo sapiens KIAA0427 gene product (KIAA0427), mRNA |
| (fragment) | | | |
| Human FBLN1 | 13 | 14 | ref\|NM_016325.1\| Homo sapiens zinc finger protein 274 (ZNF274), mRNA |
| (fragment) | | | |
| Human FBLN1 | 13 | 14 | ref\|NM_020131.1\| Homo sapiens ataxin-1 ubiquitin-like interacting protein (A1U), mRNA |
| (fragment) | | | |
| Human FBLN1 | 13 | 14 | ref\|NM_022740.1\| Homo sapiens homeodomain-interacting protein kinase 2 (HIPK2), mRNA |
| (fragment) | | | |
| Human FBLN1 | 13 | 14 | ref\|NM_024106.1\| Homo sapiens hypothetical protein MGC2663 (MGC2663), mRNA |
| (fragment) | | | |
| Human FBLN1 | 13 | 14 | ref\|NM_026107.1\| Mus musculus RIKEN cDNA 1700128E15 gene (1700128E15Rik), mRNA |
| (fragment) | | | |
| Human | 15 | 16 | dbj\|AK024505.1\|AK024505 Homo sapiens mRNA for FLJ00115 protein, partial cds |
| PSMD8 (full- | | | |
| length) | | | |
| Human | 15 | 16 | dbj\|AK026124.1\|AK026124 Homo sapiens cDNA: FLJ22471 fis, clone HRC10529 |
| PSMD8 (full- | | | |
| length) | | | |
| Human | 15 | 16 | gb\|AF151029.1\|AF151029 Homo sapiens HSPC195 mRNA, complete cds |
| PSMD8 (full- | | | |
| length) | | | |
| Human | 15 | 16 | ref\|NM_004421.1\| Homo sapiens dishevelled 1 (homologous to Drosophila dsh) (DVL1), mRNA |
| PSMD8 (full- | | | |
| length) | | | |
| Human | 15 | 16 | ref\|NM_004422.1\| Homo sapiens dishevelled 2 (homologous to Drosophila dsh) (DVL2), mRNA |
| PSMD8 (full- | | | |
| length) | | | |
| Human | 15 | 16 | ref\|NM_004423.2\| Homo sapiens dishevelled 3 (homologous to Drosophila dsh) (DVL3), mRNA |
| PSMD8 (full- | | | |
| length) | | | |
| Human | 15 | 16 | ref\|NM_005088.1\| Homo sapiens DNA segment on chromosome X and Y (unique) 155 expressed sequence |
| PSMD8 (full- | | | (DXYS155E), mRNA |
| length) | | | |
| Human | 15 | 16 | ref\|NM_015897.1\| Homo sapiens protein inhibitor of activated STAT protein PIASy (PIASY), mRNA |
| PSMD8 (full- | | | |
| length) | | | |
| Human IFITM3 | 17 | 18 | dbj\|AB002313.1\|AB002313 Human mRNA for KIAA0315 gene, |
| (fragment) | | | partial cds |
| Human IFITM3 | 17 | 18 | dbj\|AB002344.1\|AB002344 Human mRNA for KIAA0346 gene, |
| (fragment) | | | partial cds |
| Human IFITM3 | 17 | 18 | dbj\|AB011541.1\|AB011541 Homo sapiens mRNA for MEGF8, |
| (fragment) | | | partial cds |
| Human IFITM3 | 17 | 18 | dbj\|AK022425.1\|AK022425 Homo sapiens cDNA FLJ12363 fis, |
| (fragment) | | | clone MAMMA1002380 |
| Human IFITM3 | 17 | 18 | dbj\|AK024090.1\|AK024090 Homo sapiens cDNA FLJ14028 fis, |
| (fragment) | | | clone HEMBA1003838 |
| Human IFITM3 | 17 | 18 | dbj\|AP000501.1\|AP000501 Homo sapiens genomic DNA, |
| (fragment) | | | chromosome 8p11.2, clone: 91h23 to 9-41 |
| Human IFITM3 | 17 | 18 | emb\|AJ007398.1\|HSA7398 Homo sapiens mRNA for |
| (fragment) | | | PBK1 protein |
| Human IFITM3 | 17 | 18 | emb\|AL445647.6\|AL445647 Human DNA sequence from clone RP11-471M2 on chromosome 13, complete |
| (fragment) | | | sequence [Homo sapiens] |
| Human IFITM3 | 17 | 18 | emb\|X68742.1\|HSINTASA H. sapiens mRNA for integrin, alpha subunit |
| (fragment) | | | |
| Human IFITM3 | 17 | 18 | gb\|AC002553.1\|AC002553 Homo sapiens chromosome 17, clone hCIT529I10, complete sequence |
| (fragment) | | | |
| Human IFITM3 | 17 | 18 | gb\|AC011444.5\|AC011444 Homo sapiens chromosome 19 clone CTC-232P5, complete sequence |
| (fragment) | | | |
| Human IFITM3 | 17 | 18 | gb\|U42457.1\|HSU42457 Human fibronectin (FN1) mRNA, splice variant not containing EIIIB domain, partial cds |
| (fragment) | | | |
| Human IFITM3 | 17 | 18 | gb\|U81002.1\|HSU81002 Homo sapiens TRAF4 associated factor 1 mRNA, partial cds |
| (fragment) | | | |
| Human IFITM3 | 17 | 18 | ref\|NM_000064.1\| Homo sapiens complement component 3 (C3), mRNA |
| (fragment) | | | |
| Human IFITM3 | 17 | 18 | ref\|NM_000088.1\| Homo sapiens collagen, type I, alpha 1 (COL1A1), mRNA |
| (fragment) | | | |
| Human IFITM3 | 17 | 18 | ref\|NM_001456.1\| Homo sapiens filamin A, alpha (actin-binding protein-280) (FLNA), mRNA |
| (fragment) | | | |
| Human IFITM3 | 17 | 18 | ref\|NM_001457.1\| Homo sapiens filamin B, beta (actin-binding protein-278) (FLNB), mRNA |
| (fragment) | | | |
| Human IFITM3 | 17 | 18 | ref\|NM_001458.1\| Homo sapiens filamin C, gamma (actin-binding protein-280) (FLNC), mRNA |
| (fragment) | | | |
| Human IFITM3 | 17 | 18 | ref\|NM_002508.1\| Homo sapiens nidogen (enactin) (NID), mRNA |
| (fragment) | | | |
| Human IFITM3 | 17 | 18 | ref\|NM_002593.2\| Homo sapiens procollagen C-endopeptidase enhancer (PCOLCE), mRNA |
| (fragment) | | | |
| Human IFITM3 | 17 | 18 | ref\|NM_002815.1\| Homo sapiens proteasome (prosome, macropain) 26S subunit, non-ATPase, 11 (PSMD11), |
| (fragment) | | | mRNA |
| Human IFITM3 | 17 | 18 | ref\|NM_002844.1\| Homo sapiens protein tyrosine phosphatase, receptor type, K (PTPRK), mRNA |
| (fragment) | | | |
| Human IFITM3 | 17 | 18 | ref\|NM_002950.1\| Homo sapiens ribophorin I (RPN1), mRNA |
| (fragment) | | | |
| Human IFITM3 | 17 | 18 | ref\|NM_002951.1\| Homo sapiens ribophorin II (RPN2), mRNA |
| (fragment) | | | |
| Human IFITM3 | 17 | 18 | ref\|NM_003345.1\| Homo sapiens ubiquitin-conjugating enzyme E2I (homologous to yeast UBC9) (UBE2I), mRNA |
| (fragment) | | | |
| Human IFITM3 | 17 | 18 | ref\|NM_003461.1\| Homo sapiens zyxin (ZYX), mRNA |
| (fragment) | | | |
| Human IFITM3 | 17 | 18 | ref\|NM_004068.1\| Homo sapiens adaptor-related protein complex 2, mu 1 subunit (AP2M1), mRNA |
| (fragment) | | | |
| Human IFITM3 | 17 | 18 | ref\|NM_004431.1\| Homo sapiens EphA2 (EPHA2), mRNA |
| (fragment) | | | |
| Human IFITM3 | 17 | 18 | ref\|NM_005385.2\| Homo sapiens natural killer-tumor recognition sequence (NKTR), mRNA |
| (fragment) | | | |
| Human IFITM3 | 17 | 18 | ref\|NM_005396.1\| Homo sapiens pancreatic lipase-related protein 2 (PNLIPRP2), mRNA |
| (fragment) | | | |
| Human IFITM3 | 17 | 18 | ref\|NM_005433.1\| Homo sapiens v-yes-1 Yamaguchi sarcoma viral oncogene homolog 1 (YES1), mRNA |
| (fragment) | | | |
| Human IFITM3 | 17 | 18 | ref\|NM_006519.1\| Homo sapiens t-complex-associated-testis-expressed 1-like 1 (TCTEL1), mRNA |
| (fragment) | | | |
| Human IFITM3 | 17 | 18 | ref\|NM_007184.1\| Homo sapiens imidazoline receptor candidate (I-1), mRNA |
| (fragment) | | | |
| Human IFITM3 | 17 | 18 | ref\|NM_014242.1\| Homo sapiens zinc finger protein 237 (ZNF237), mRNA |
| (fragment) | | | |
| Human IFITM3 | 17 | 18 | ref\|NM_014819.1\| Homo sapiens KIAA0438 gene product (KIAA0438), mRNA |
| (fragment) | | | |
| Human IFITM3 | 17 | 18 | ref\|NM_017797.1\| Homo sapiens hypothetical protein FLJ20386 (FLJ20386), mRNA |
| (fragment) | | | |
| Human IFITM3 | 17 | 18 | ref\|NM_018200.1\| Homo sapiens high-mobility group 20A (HMG20A), mRNA |
| (fragment) | | | |
| Human IFI616 | 21 | 22 | dbj\|AB007857.2\|AB007857 Homo sapiens mRNA for KIAA0397 protein, partial cds |
| (fragment) | | | |
| Human IFI616 | 21 | 22 | dbj\|AB014566.1\|AB014566 Homo sapiens mRNA for KIAA0666 protein, partial cds |
| (fragment) | | | |
| Human IFI616 | 21 | 22 | dbj\|AB037728.1\|AB037728 Homo sapiens mRNA for KIAA1307 protein, partial cds |
| (fragment) | | | |
| Human IFI616 | 21 | 22 | dbj\|AB037802.1\|AB037802 Homo sapiens mRNA for KIAA1381 protein, partial cds |
| (fragment) | | | |
| Human IFI616 | 21 | 22 | dbj\|AB037856.1\|AB037856 Homo sapiens mRNA for KIAA1435 protein, partial cds |
| (fragment) | | | |
| Human IFI616 | 21 | 22 | emb\|AL121934.17\|HSBA209A2 Human DNA sequence from clone RP11-209A2 on chromosome 6. Contains an |
| (fragment) | | | RPL10 (60S ribosomal protein L10) pseudogene, ESTs, STSs and GSSs, complete sequence [Homo sapiens] |
| Human IFI616 | 21 | 22 | emb\|AL133246.2\|CNS01DUJ BAC sequence from the SPG4 candidate region at 2p21-2p22 BAC 852C13 of |
| (fragment) | | | library RPCI-11 from chromosome 2 of Homo sapiens (Human), complete sequence |
| Human IFI616 | 21 | 22 | emb\|AL137738.1\|HSM802321 Homo sapiens mRNA; cDNA DKFZp564M2163 (from clone DKFZp564M2163) |
| (fragment) | | | |
| Human IFI616 | 21 | 22 | emb\|AL158830.17\|AL158830 Human DNA sequence from clone RP11-421H8 on chromosome 9, complete |
| (fragment) | | | sequence [Homo sapiens] |
| Human IFI616 | 21 | 22 | emb\|X92474.1\|HSCHTOG H. sapiens mRNA for ch-TOG protein |
| (fragment) | | | |
| Human IFI616 | 21 | 22 | emb\|Z75887.1\|HS130N4 Human DNA sequence from PAC 130N4, BRCA2 gene region chromosome 13q12-13 |
| (fragment) | | | contains xs7 mRNA, ESTs |
| Human IFI616 | 21 | 22 | gb\|AC002519.1\|HUAC002519 Human Chromosome 16 BAC clone CIT987SK-A-355G7, complete sequence |
| (fragment) | | | [Homo sapiens] |
| Human IFI616 | 21 | 22 | gb\|AC005669.1\|AC005669 Homo sapiens chromosome 3, clone hRPK.165_I_16, complete sequence |
| (fragment) | | | |
| Human IFI616 | 21 | 22 | gb\|AC010183.6\|AC010183 Homo sapiens 12q BAC RP11-100F15 (Roswell Park Cancer Institute Human BAC |
| (fragment) | | | Library) complete sequence |
| Human IFI616 | 21 | 22 | gb\|AC011891.3\|AC011891 Homo sapiens clone CTD-2320J21, complete sequence |
| (fragment) | | | |
| Human IFI616 | 21 | 22 | gb\|AC012150.16\|AC012150 Homo sapiens 12 BAC RP11-946L16 (Roswell Park Cancer Institute Human BAC |
| (fragment) | | | Library) complete sequence |
| Human IFI616 | 21 | 22 | gb\|AC018647.4\|AC018647 Homo sapiens chromosome 7 clone RP11-379H18, complete sequence |
| (fragment) | | | |
| Human IFI616 | 21 | 22 | gb\|AC026400.3\|AC026400 Homo sapiens chromosome 5 clone CTB-173O12, complete sequence |
| (fragment) | | | |
| Human IFI616 | 21 | 22 | gb\|AC068726.5\|AC068726 Homo sapiens chromosome 15 clone RP11-844G16 map 15q21.3, complete |
| (fragment) | | | sequence |
| Human IFI616 | 21 | 22 | gb\|AF002223.1\|AF002223 Human genomic DNA of Xq28 with MTM1 and MTMR1 genes, complete sequence |
| (fragment) | | | [Homo sapiens] |
| Human IFI616 | 21 | 22 | gb\|AF040965.1\|AF040965 Homo sapiens unknown protein IT12 mRNA, partial cds |
| (fragment) | | | |
| Human IFI616 | 21 | 22 | gb\|AF129756.1\|DJ201G24 Homo sapiens MSH55 gene, partial cds; and CLIC1, DDAH, G6b, G6c, G5b, G6d, |
| (fragment) | | | G6e, G6f, BAT5, G5b, CSK2B, BAT4, G4, Apo M, BAT3, BAT2, AIF-1, 1C7, LST-1, LTB, TNF, and LTA genes, |
| | | | complete cds |
| Human IFI616 | 21 | 22 | gb\|AF132362.1\|AF132362 Homo sapiens hnRNP 2H9B mRNA, complete cds |
| (fragment) | | | |
| Human IFI616 | 21 | 22 | gb\|AF141884.1\|AF141884 Homo sapiens oligophrenin-1 like protein (OPHN1L) mRNA, complete cds |
| (fragment) | | | |
| Human IFI616 | 21 | 22 | gb\|AF168787.1\|AF168787 Homo sapiens vanilloid receptor gene, partial sequence; CARKL and CTNS genes, |
| (fragment) | | | complete cds; TIP1 gene, partial cds; P2X5b and P2X5a genes, complete cds; and HUMINAE gene, partial cds |
| Human IFI616 | 21 | 22 | gb\|BC001096.1\|BC001096 Homo sapiens, clone IMAGE: 3507281, mRNA, partial cds |
| (fragment) | | | |
| Human IFI616 | 21 | 22 | gb\|U77700.1\|HSU77700 Homo sapiens HsGCN1 mRNA, partial cds |
| (fragment) | | | |
| Human IFI616 | 21 | 22 | ref\|NM_000034.1\| Homo sapiens aldolase A, fructose-bisphosphate (ALDOA), mRNA |
| (fragment) | | | |
| Human IFI616 | 21 | 22 | ref\|NM_000296.1\| Homo sapiens polycystic kidney disease 1 (autosomal dominant) (PKD1), mRNA |
| (fragment) | | | |
| Human IFI616 | 21 | 22 | ref\|NM_000445.1\| Homo sapiens plectin 1, intermediate filament binding protein, 500 kD (PLEC1), mRNA |
| (fragment) | | | |
| Human IFI616 | 21 | 22 | ref\|NM_001034.1\| Homo sapiens ribonucleotide reductase M2 polypeptide (RRM2), mRNA |
| (fragment) | | | |
| Human IFI616 | 21 | 22 | ref\|NM_001288.2\| Homo sapiens chloride intracellular channel 1 (CLIC1), mRNA |
| (fragment) | | | |
| Human IFI616 | 21 | 22 | ref\|NM_001316.1\| Homo sapiens chromosome segregation 1 (yeast homolog)-like (CSE1L), mRNA |
| (fragment) | | | |
| Human IFI616 | 21 | 22 | ref\|NM_001745.1\| Homo sapiens calcium modulating ligand (CAMLG), mRNA |
| (fragment) | | | |
| Human IFI616 | 21 | 22 | ref\|NM_001833.1\| Homo sapiens clathrin, light polypeptide (Lca) (CLTA), transcript variant nonbrain, mRNA |
| (fragment) | | | |
| Human IFI616 | 21 | 22 | ref\|NM_002265.1\| Homo sapiens karyopherin (importin) beta 1 (KPNB1), mRNA |
| (fragment) | | | |
| Human IFI616 | 21 | 22 | ref\|NM_002291.1\| Homo sapiens laminin, beta 1 (LAMB1), mRNA |
| (fragment) | | | |
| Human IFI616 | 21 | 22 | ref\|NM_002807.1\| Homo sapiens proteasome (prosome, macropain) 26S subunit, non-ATPase, 1 (PSMD1), |
| (fragment) | | | mRNA |
| Human IFI616 | 21 | 22 | ref\|NM_002841.1\| Homo sapiens protein tyrosine phosphatase, receptor type, G (PTPRG), mRNA |
| (fragment) | | | |
| Human IFI616 | 21 | 22 | ref\|NM_002857.1\| Homo sapiens peroxisomal farnesylated protein (PXF), mRNA |
| (fragment) | | | |
| Human IFI616 | 21 | 22 | ref\|NM_003404.1\| Homo sapiens tyrosine 3-monooxygenase/tryptophan 5-monooxygenase activation protein, beta |
| (fragment) | | | polypeptide (YWHAB), mRNA |
| Human IFI616 | 21 | 22 | ref\|NM_004039.1\| Homo sapiens annexin A2 (ANXA2), mRNA |
| (fragment) | | | |
| Human IFI616 | 21 | 22 | ref\|NM_004184.2\| Homo sapiens tryptophanyl-tRNA synthetase (WARS), mRNA |
| (fragment) | | | |
| Human IFI616 | 21 | 22 | ref\|NM_004264.1\| Homo sapiens SRB7 (suppressor of RNA polymerase B, yeast) homolog (SURB7), mRNA |
| (fragment) | | | |
| Human IFI616 | 21 | 22 | ref\|NM_004317.1\| Homo sapiens arsA (bacterial) arsenite transporter, ATP-binding, homolog 1 (ASNA1), mRNA |
| (fragment) | | | |
| Human IFI616 | 21 | 22 | ref\|NM_004318.1\| Homo sapiens aspartate beta-hydroxylase (ASPH), mRNA |
| (fragment) | | | |
| Human IFI616 | 21 | 22 | ref\|NM_004370.3\| Homo sapiens collagen, type XII, alpha 1 (COL12A1), mRNA |
| (fragment) | | | |
| Human IFI616 | 21 | 22 | ref\|NM_004381.1\| Homo sapiens cAMP responsive element binding protein-like 1 (CREBL1), mRNA |
| (fragment) | | | |
| Human IFI616 | 21 | 22 | ref\|NM_004523.1\| Homo sapiens kinesin-like 1 (KNSL1), mRNA |
| (fragment) | | | |
| Human IFI616 | 21 | 22 | ref\|NM_004639.1\| Homo sapiens HLA-B associated transcript-3 (D6S52E), mRNA |
| (fragment) | | | |
| Human IFI616 | 21 | 22 | ref\|NM_004652.2\| Homo sapiens ubiquitin specific protease 9, X chromosome (Drosophila fat facets related) |
| (fragment) | | | (USP9X), transcript variant 1, mRNA |
| Human IFI616 | 21 | 22 | ref\|NM_005406.1\| Homo sapiens Rho-associated, coiled-coil containing protein kinase 1 (ROCK1), mRNA |
| (fragment) | | | |
| Human IFI616 | 21 | 22 | ref\|NM_005625.1\| Homo sapiens syndecan binding protein (syntenin) (SDCBP), mRNA |
| (fragment) | | | |
| Human IFI616 | 21 | 22 | ref\|NM_005880.2\| Homo sapiens DnaJ (Hsp40) homolog, subfamily A, member 2 (DNAJA2), mRNA |
| (fragment) | | | |
| Human IFI616 | 21 | 22 | ref\|NM_005996.1\| Homo sapiens T-box 3 (ulnar mammary syndrome) (TBX3), mRNA |
| (fragment) | | | |
| Human IFI616 | 21 | 22 | ref\|NM_005998.1\| Homo sapiens chaperonin containing TCP1, subunit 3 (gamma) (CCT3), mRNA |
| (fragment) | | | |
| Human IFI616 | 21 | 22 | ref\|NM_006384.2\| Homo sapiens calcium and integrin binding protein (DNA-dependent protein kinase interacting |
| (fragment) | | | protein) (SIP2-28), mRNA |
| Human IFI616 | 21 | 22 | ref\|NM_006421.2\| Homo sapiens brefeldin A-inhibited guanine nucleotide-exchange protein 1 (BIG1), mRNA |
| (fragment) | | | |
| Human IFI616 | 21 | 22 | ref\|NM_006429.1\| Homo sapiens chaperonin containing TCP1, subunit 7 (eta) (CCT7), mRNA |
| (fragment) | | | |
| Human IFI616 | 21 | 22 | ref\|NM_006621.1\| Homo sapiens S-adenosylhomocysteine hydrolase-like 1 (AHCYL1), mRNA |
| (fragment) | | | |
| Human IFI616 | 21 | 22 | ref\|NM_006660.3\| Homo sapiens ClpX (caseinolytic protease X, E. coli ) homolog (CLPX), mRNA |
| (fragment) | | | |
| Human IFI616 | 21 | 22 | ref\|NM_006788.1\| Homo sapiens ralA binding protein 1 (RALBP1), mRNA |
| (fragment) | | | |
| Human IFI616 | 21 | 22 | ref\|NM_014423.1\| Homo sapiens ALL1 fused gene from 5q31 (AF5Q31), mRNA |
| (fragment) | | | |
| Human IFI616 | 21 | 22 | ref\|NM_014437.1\| Homo sapiens zinc/iron regulated transporter-like (ZIRTL), mRNA |
| (fragment) | | | |
| Human IFI616 | 21 | 22 | ref\|NM_015456.1\| Homo sapiens DKFZP586B0519 protein (DKFZP586B0519), mRNA |
| (fragment) | | | |
| Human IFI616 | 21 | 22 | ref\|NM_015888.1\| Homo sapiens hook1 protein (HOOK1), mRNA |
| (fragment) | | | |
| Human IFI616 | 21 | 22 | ref\|NM_018157.1\| Homo sapiens hypothetical protein FLJ10620 (FLJ10620), mRNA |
| (fragment) | | | |
| Human IFI616 | 21 | 22 | ref\|NM_018200.1\| Homo sapiens high-mobility group 20A (HMG20A), mRNA |
| (fragment) | | | |
| Human IFI616 | 21 | 22 | ref\|NM_021430.1\|Mus musculus hypothetical protein (LOC58241), mRNA |
| (fragment) | | | |
| Human IFI616 | 23 | 24 | dbj\|AB023164.1\|AB023164 Homo sapiens mRNA for KIAA0947 protein, partial cds |
| (fragment) | | | |
| Human IFI616 | 23 | 24 | dbj\|AB023164.1\|AB023164 Homo sapiens mRNA for KIAA0947 protein, partial cds |
| (fragment) | | | |
| Human IFI616 | 23 | 24 | dbj\|AB037858.1\|AB037858 Homo sapiens mRNA for KIAA1437 protein, partial cds |
| (fragment) | | | |
| Human IFI616 | 23 | 24 | dbj\|AB046782.1\|AB046782 Homo sapiens mRNA for KIAA1562 protein, partial cds |
| (fragment) | | | |
| Human IFI616 | 23 | 24 | dbj\|AB046814.1\|AB046814 Homo sapiens mRNA for KIAA1594 protein, partial cds |
| (fragment) | | | |
| Human IFI616 | 23 | 24 | dbj\|AK022708.1\|AK022708 Homo sapiens cDNA FLJ12646 fis, clone NT2RM4001987, weakly similar to NEURAL |
| (fragment) | | | CELL ADHESION MOLECULE 1, LARGE ISOFORM PRECURSOR |
| Human IFI616 | 23 | 24 | dbj\|AK024508.1\|AK024508 Homo sapiens mRNA for FLJ00118 protein, partial cds |
| (fragment) | | | |
| Human IFI616 | 23 | 24 | dbj\|D85433.1\|D85433 Human MURR1 mRNA, sequence |
| (fragment) | | | |
| Human IFI616 | 23 | 24 | emb\|AL031669.28\|HS661I20 Human DNA sequence from clone RP4-661I20 on chromosome 20q11.23-12 |
| (fragment) | | | Contains a gene for a protein similar to RPL12 (ribosomal protein L12), part of a novel Helicase C-terminal |
| | | | domain and SNF2 N-terminal domains containing protein, ESTs, STSs, GSSs and C |
| Human IFI616 | 23 | 24 | emb\|AL080168.1\|HSM800687 Homo sapiens mRNA; cDNA DKFZp434C151 (from clone DKFZp434C151); |
| (fragment) | | | complete cds |
| Human IFI616 | 23 | 24 | emb\|AL109613.11\|HS1033H22 Human DNA sequence from clone 1033H22 on chromosome 1p21.2-22.2, |
| (fragment) | | | complete sequence [Homo sapiens] |
| Human IFI616 | 23 | 24 | emb\|AL133355.12\|AL133355 Human DNA sequence from clone RP11-541N10 on chromosome 10. Contains a |
| (fragment) | | | novel gene and the 5′ end of the gene for a novel protein (ortholog of mouse FISH protein), ESTs, STSs, GSSs |
| | | | and two putative CpG islands, complete sequence [Homo sapiens] |
| Human IFI616 | 23 | 24 | emb\|AL135858.2\|CNS01DVJ Human chromosome 14 DNA sequence *** IN PROGRESS *** BAC R-667E4 of |
| (fragment) | | | library RPCI-11 from chromosome 14 of Homo sapiens (Human), complete sequence |
| Human IFI616 | 23 | 24 | emb\|AL137471.1\|HSM802190 Homo sapiens mRNA; cDNA DKFZp434B0923 (from clone DKFZp434B0923); |
| (fragment) | | | partial cds |
| Human IFI616 | 23 | 24 | emb\|AL138784.30\|AL138784 Human DNA sequence from clone RP5-1102M4 on chromosome 1, complete |
| (fragment) | | | sequence [Homo sapiens] |
| Human IFI616 | 23 | 24 | emb\|AL161450.14\|AL161450 Human DNA sequence from clone RP11-39K24 on chromosome 9, complete |
| (fragment) | | | sequence [Homo sapiens] |
| Human IFI616 | 23 | 24 | emb\|AL161799.19\|AL161799 Human DNA sequence from clone RP11-177H22 on chromosome 10, complete |
| (fragment) | | | sequence [Homo sapiens] |
| Human IFI616 | 23 | 24 | emb\|AL162455.14\|AL162455 Human DNA sequence from clone RP11-62D23 on chromosome 13, complete |
| (fragment) | | | sequence [Homo sapiens] |
| Human IFI616 | 23 | 24 | emb\|AL353688.7\|AL353688 Human DNA sequence from clone RP11-275O3 on chromosome 13 Contains GSSs, |
| (fragment) | | | complete sequence [Homo sapiens] |
| Human IFI616 | 23 | 24 | emb\|AL365505.15\|AL365505 Human DNA sequence from clone RP11-382A12 on chromosome 20 Contains |
| (fragment) | | | ESTs, STSs, GSSs and CpG islands. Contains the 5′ part of the gene for a 72.1 KDa protein (DKFZP564A032, |
| | | | SBBI88) similar to mouse IFN-gamma induced MG11 and the 3′ part of the RBL1 gen |
| Human IFI616 | 23 | 24 | emb\|AL450224.1\|HSBC17A96 Homo sapiens chromosome 17 sequence from PAC RPCI-5 926L12 map 17q13.3 |
| (fragment) | | | region D17S695-D17S654, complete sequence |
| Human IFI616 | 23 | 24 | emb\|X02761.1\|HSFIB1 Human mRNA for fibronectin (FN precursor) |
| (fragment) | | | |
| Human IFI616 | 23 | 24 | emb\|Z75889.1\|HS267P19 Human DNA sequence from cosmid 267P19, BRCA2 gene region chromosome |
| (fragment) | | | 13q12-13 contains polymorphic CA repeat |
| Human IFI616 | 23 | 24 | emb\|Z80107.1\|HS197J16 Human DNA sequence from PAC 197J16, between markers DXS366 and DXS87 on |
| (fragment) | | | chromosome X |
| Human IFI616 | 23 | 24 | emb\|Z83070.1\|HSZ83070 H. sapiens Fanconi anaemia group A gene, exons 4 and 5 |
| (fragment) | | | |
| Human IFI616 | 23 | 24 | emb\|Z97832.11\|HS329A5 Human DNA sequence from clone RP3-329A5 on chromosome 6p21.1-21.33 Contains |
| (fragment) | | | a pseudogene similar to ribosomal protein L35a, ZNF76 (zinc finger protein 76 (expressed in testis)), part of the |
| | | | gene for KIAA06460 protein, an EST, STSs, GSSs and CpG Islands |
| Human IFI616 | 23 | 24 | gb\|AC002390.1\|AC002390 Human DNA from overlapping chromosome 19-specific cosmids R30072 and |
| (fragment) | | | R28588, genomic sequence, complete sequence [Homo sapiens] |
| Human IFI616 | 23 | 24 | gb\|AC003042.1\|AC003042 Homo sapiens chromosome 17, clone HCIT75G16, complete sequence |
| (fragment) | | | |
| Human IFI616 | 23 | 24 | gb\|AC004241.1\|AC004241 Homo sapiens 12q13.1 PAC RPCI3-197B17 (Roswell Park Cancer Institute Human |
| (fragment) | | | PAC library) complete sequence |
| Human IFI616 | 23 | 24 | gb\|AC004560.1\|AC004560 Homo sapiens chromosome 19, cosmid R30676, complete sequence |
| (fragment) | | | |
| Human IFI616 | 23 | 24 | gb\|AC004757.1\|AC004757 Homo sapiens chromosome 17, clone hCIT.117_K_16, complete sequence |
| (fragment) | | | |
| Human IFI616 | 23 | 24 | gb\|AC005037.2\|AC005037 Homo sapiens BAC clone RP11-469M7 from 2, complete sequence |
| (fragment) | | | |
| Human IFI616 | 23 | 24 | gb\|AC005335.1\|AC005335 Homo sapiens chromosome 19, cosmid R34296, complete sequence |
| (fragment) | | | |
| Human IFI616 | 23 | 24 | gb\|AC005342.1\|AC005342 Homo sapiens 12p13.3 PAC RPCI5-1096D14 (Roswell Park Cancer Institute Human |
| (fragment) | | | PAC Library) complete sequence |
| Human IFI616 | 23 | 24 | gb\|AC005484.2\|AC005484 Homo sapiens PAC clone RP5-847O8 from 14q24.3, complete sequence |
| (fragment) | | | |
| Human IFI616 | 23 | 24 | gb\|AC005529.7\|AC005529 Homo sapiens chromosome 22q12 clone bk256d12, complete sequence |
| (fragment) | | | |
| Human IFI616 | 23 | 24 | gb\|AC005899.1\|AC005899 Homo sapiens chromosome 17, clone hRPK.227_G_15, complete sequence |
| (fragment) | | | |
| Human IFI616 | 23 | 24 | gb\|AC006430.22\|AC006430 Homo sapiens chromosome 9, clone RP11-525G7, complete sequence |
| (fragment) | | | |
| Human IFI616 | 23 | 24 | gb\|AC006475.3\|AC006475 Homo sapiens PAC clone RP4-727L20 from 7q36, complete sequence |
| (fragment) | | | |
| Human IFI616 | 23 | 24 | gb\|AC007240.2\|AC007240 Homo sapiens BAC clone RP11-83M8 from 2, complete sequence |
| (fragment) | | | |
| Human IFI616 | 23 | 24 | gb\|AC007376.9\|AC007376 Homo sapiens chromosome 14 clone 99E15 containing gene for KIAA 1036, |
| (fragment) | | | complete CDS, complete sequence |
| Human IFI616 | 23 | 24 | gb\|AC007619.22\|AC007619 Homo sapiens 12p BAC RP11-253I19 (Roswell Park Cancer Institute Human BAC |
| (fragment) | | | Library) complete sequence |
| Human IFI616 | 23 | 24 | gb\|AC008122.15\|AC008122 Homo sapiens 2q31-33 BAC RPCI11-1021H16 (Roswell Park Cancer Institute |
| (fragment) | | | Human BAC Library) complete sequence |
| Human IFI616 | 23 | 24 | gb\|AC010083.5\|AC010083 Homo sapiens BAC clone RP11-125L14 from 2, complete sequence |
| (fragment) | | | |
| Human IFI616 | 23 | 24 | gb\|AC016620.6\|AC016620 Homo sapiens chromosome 5 clone CTD-2318A17, complete sequence |
| (fragment) | | | |
| Human IFI616 | 23 | 24 | gb\|AC018710.4\|AC018710 Homo sapiens clone RP11-296L20, complete sequence |
| (fragment) | | | |
| Human IFI616 | 23 | 24 | gb\|AC024939.24\|AC024939 Homo sapiens 12 BAC RP11-485K18 (Roswell Park Cancer institute Human BAC |
| (fragment) | | | Library) complete sequence |
| Human IFI616 | 23 | 24 | gb\|AC026126.19\|AC026126 Homo sapiens 12 BAC RP11-19K10 (Roswell Park Cancer Institute Human BAC |
| (fragment) | | | Library) complete sequence |
| Human IFI616 | 23 | 24 | gb\|AC046129.17\|AC046129 Homo sapiens 12 BAC RP11-150C16 (Roswell Park Cancer Institute Human BAC |
| (fragment) | | | Library) complete sequence |
| Human IFI616 | 23 | 24 | gb\|AF001552.1\|HSAF001552 Homo sapiens chromosome 16 BAC clone CIT987SK-381E11 complete sequence |
| (fragment) | | | |
| Human IFI616 | 23 | 24 | gb\|AF001892.1\|BETA1 Human MEN1 region clone epsilon/beta mRNA, 5′ fragment |
| (fragment) | | | |
| Human IFI616 | 23 | 24 | gb\|AF018034.1\|AF018034 Homo sapiens endothelin converting enzyme-1 (ECE-1) gene, exon 19, partial cds |
| (fragment) | | | |
| Human IFI616 | 23 | 24 | gb\|AF038564.1\|AF038564 Homo sapiens atrophin-1 interacting protein 4 (AIP4) mRNA, partial cds |
| (fragment) | | | |
| Human IFI616 | 23 | 24 | gb\|AF045555.1\|AF045555 Homo sapiens wbscr1 (WBSCR1) and wbscr5 (WBSCR5) genes, complete cds, |
| (fragment) | | | alternatively spliced and replication factor C subunit 2 (RFC2) gene, complete cds |
| Human IFI616 | 23 | 24 | gb\|AF200348.1\|AF200348 Homo sapiens melanoma-associated antigen MG50 mRNA, partial cds |
| (fragment) | | | |
| Human IFI616 | 23 | 24 | gb\|AF216292.1\|AF216292 Homo sapiens endoplasmic reticulum lumenal Ca2+ binding protein grp78 mRNA, |
| (fragment) | | | complete cds |
| Human IFI616 | 23 | 24 | gb\|AF279660.2\|AF279660 Homo sapiens CAR (RFP2) gene, complete cds; DLEU2 and DLEU1 genes, complete |
| (fragment) | | | sequence; and RPL18 and p48/Hip pseudogenes, complete sequence |
| Human IFI616 | 23 | 24 | gb\|AY007103.1\| Homo sapiens clone TCCCIA00297 mRNA sequence |
| (fragment) | | | |
| Human IFI616 | 23 | 24 | gb\|BC002484.1\|BC002484 Homo sapiens, clone MGC: 3080, mRNA, complete cds |
| (fragment) | | | |
| Human IFI616 | 23 | 24 | gb\|BC003391.1\|BC003391 Homo sapiens, clone IMAGE: 3447324, mRNA, partial cds |
| (fragment) | | | |
| Human IFI616 | 23 | 24 | gb\|L78833.1\|HUMBRCA1 Human BRCA1, Rho7 and vatl genes, complete cds, and ipf35 gene, partial cds |
| (fragment) | | | |
| Human IFI616 | 23 | 24 | gb\|U42594.1\|HSU42594 Human fibronectin (FN1) mRNA, splice variant, partial cds |
| (fragment) | | | |
| Human IFI616 | 23 | 24 | ref\|NM_000094.1\| Homo sapiens collagen, type VII, alpha 1 (epidermolysis bullosa, dystrophic, dominant and |
| (fragment) | | | recessive) (COL7A1), mRNA |
| Human IFI616 | 23 | 24 | ref\|NM_001286.1\| Homo sapiens chloride channel 6 (CLCN6), transcript variant CIC-6a, mRNA |
| (fragment) | | | |
| Human IFI616 | 23 | 24 | ref\|NM_001431.1\| Homo sapiens erythrocyte membrane protein band 4.1-like 2 (EPB41L2), mRNA |
| (fragment) | | | |
| Human IFI616 | 23 | 24 | ref\|NM_001990.1\| Homo sapiens eyes absent (Drosophila) homolog 3 (EYA3), mRNA |
| (fragment) | | | |
| Human IFI616 | 23 | 24 | ref\|NM_002139.1\| Homo sapiens RNA binding motif protein, X chromosome (RBMX), mRNA |
| (fragment) | | | |
| Human IFI616 | 23 | 24 | ref\|NM_002705.1\| Homo sapiens periplakin (PPL), mRNA |
| (fragment) | | | |
| Human IFI616 | 23 | 24 | ref\|NM_002734.1\| Homo sapiens protein kinase, cAMP-dependent, regulatory, type I, alpha (tissue specific |
| (fragment) | | | extinguisher 1) (PRKAR1A), mRNA |
| Human IFI616 | 23 | 24 | ref\|NM_002815.1\| Homo sapiens proteasome (prosome, macropain) 26S subunit, non-ATPase, 11 (PSMD11), |
| (fragment) | | | mRNA |
| Human IFI616 | 23 | 24 | ref\|NM_002842.1\| Homo sapiens protein tyrosine phosphatase, receptor type, H (PTPRH), mRNA |
| (fragment) | | | |
| Human IFI616 | 23 | 24 | ref\|NM_005245.1\| Homo sapiens FAT tumor suppressor (Drosophila) homolog (FAT), mRNA |
| (fragment) | | | |
| Human IFI616 | 23 | 24 | ref\|NM_006582.2\| Homo sapiens glucocorticoid modulatory element binding protein 1 (GMEB1), transcript variant |
| (fragment) | | | 1, mRNA |
| Human IFI616 | 23 | 24 | ref\|NM_006597.1\| Homo sapiens heat shock 70 kD protein 8 (HSPA8), mRNA |
| (fragment) | | | |
| Human IFI616 | 23 | 24 | ref\|NM_007034.2\| Homo sapiens DnaJ-like heat shock protein 40 (HLJ1), mRNA |
| (fragment) | | | |
| Human IFI616 | 23 | 24 | ref\|NM_012079.2\| Homo sapiens diacylglycerol O-acyltransferase (mouse) homolog (DGAT), mRNA |
| (fragment) | | | |
| Human IFI616 | 23 | 24 | ref\|NM_014741.1\| Homo sapiens KIAA0652 gene product (KIAA0652), mRNA |
| (fragment) | | | |
| Human IFI616 | 23 | 24 | ref\|NM_014864.1\| Homo sapiens KIAA0475 gene product (KIAA0475), mRNA |
| (fragment) | | | |
| Human IFI616 | 23 | 24 | ref\|NM_014939.1\| Homo sapiens KIAA1012 protein (KIAA1012), mRNA |
| (fragment) | | | |
| Human IFI616 | 23 | 24 | ref\|NM_018200.1\| Homo sapiens high-mobility group 20A (HMG20A), mRNA |
| (fragment) | | | |
| Human IFI616 | 23 | 24 | ref\|NM_018685.1\| Homo sapiens anillin (Drosophila Scraps homolog), actin binding protein (ANLN), mRNA |
| (fragment) | | | |
| Human IFI616 | 23 | 24 | ref\|NM_018923.1\| Homo sapiens protocadherin gamma subfamily B, 2 (PCDHGB2), mRNA |
| (fragment) | | | |
| Human IFI616 | 23 | 24 | ref\|NM_022758.1\| Homo sapiens hypothetical protein FLJ22195 (FLJ22195), mRNA |
| (fragment) | | | |
| Human IFI616 | 23 | 24 | ref\|NM_025240.1\| Homo sapiens B7 homolog 3 (B7-H3), mRNA |
| (fragment) | | | |
| Human IFI616 | 23 | 24 | ref\|XM_008277.2\| Homo sapiens nuclear receptor co-repressor 1 (NCOR1), mRNA |
| (fragment) | | | |
| Human IFI616 | 25 | 26 | emb\|X96506.1\|HSNC2ALPH H. sapiens mRNA for NC2 alpha subunit |
| (fragment) | | | |
| Human IFI616 | 25 | 26 | gb\|AC005020.5\|AC005020 Homo sapiens BAC clone GS1-259H13 from 7, complete sequence |
| (fragment) | | | |
| Human IFI616 | 25 | 26 | gb\|AC074224.3\|AC074224 Mus musculus chromosome 2 clone RP23-43C3 strain C57BL6/J, complete sequence |
| (fragment) | | | |
| Human IFI616 | 25 | 26 | gb\|AF265342.1\|AF265342 Homo sapiens chromosome 8 map 8p BAC 2053N22, complete sequence |
| (fragment) | | | |
| Human IFI616 | 25 | 26 | ref\|NM_004930.1\| Homo sapiens capping protein (actin filament) muscle Z-line, beta (CAPZB), mRNA |
| (fragment) | | | |
| Human IFI616 | 25 | 26 | ref\|NM_005935.1\| Homo sapiens myeloid/lymphoid or mixed-lineage leukemia (trithorax (Drosophila) homolog); |
| (fragment) | | | translocated to, 2 (MLLT2), mRNA |
| Human IFI616 | 25 | 26 | ref\|NM_006077.1\| Homo sapiens calcium binding atopy-related autoantigen 1 (CBARA1), mRNA |
| (fragment) | | | |
| Human IFI616 | 25 | 26 | ref\|NM_018132.1\| Homo sapiens hypothetical protein FLJ10545 (FLJ10545), mRNA |
| (fragment) | | | |
| Human IFI616 | 25 | 26 | ref\|NM_022156.1\| Homo sapiens PP3111 protein (PP3111), mRNA |
| (fragment) | | | |
| Human IFI616 | 25 | 26 | ref\|NM_022288.1\| Rattus norvegicus casein kinase 1 gamma 1 (Csnk1g1), mRNA |
| (fragment) | | | |
| Human IFI616 | 25 | 26 | ref\|XM_003963.2\| Homo sapiens arginyl-tRNA synthetase (RARS), mRNA |
| (fragment) | | | |
| Human | 42 | 43 | prey78847 |
| DVL1_v1 | | | |
| Human | 42 | 43 | prey78271 (prey67898; prey67896; prey67901; prey66163; prey66165; prey66161; |
| DVL1_v1 | | | prey67897; prey66169; prey70558; prey67899; prey67900; prey74048; prey66160; |
| | | | prey66162; prey66166; prey66167; prey66168; prey100021) |
| Human | 42 | 43 | prey78859 |
| DVL1_v1 | | | |
| Human | 42 | 43 | prey1469 (COL3A1; prey1473; prey19974; prey22635) hCOL3A1 hprepro alpha 1 type |
| DVL1_v1 | | | 3collagen hCOL3A1 |
| Human | 42 | 43 | prey68776 (SAS; prey68778) hSAS |
| DVL1_v1 | | | |
| Human | 42 | 43 | prey78867 |
| DVL1_v1 | | | |
| Human | 42 | 43 | prey78869 (prey78868) |
| DVL1_v1 | | | |
| Human | 42 | 43 | prey78871 (TMPRSS3 DFNB10 DFNB8 ECHOS1 TADG12; prey78875) hTMPRSS3 hTMPRSS3d |
| DVL1_v1 | | | |
| Human | 42 | 43 | prey78877 |
| DVL1_v1 | | | |
| Human | 42 | 43 | prey78879 |
| DVL1_v1 | | | |
| Human | 42 | 43 | prey78890 (prey78889) |
| DVL1_v1 | | | |
| Human | 42 | 43 | prey78891 |
| DVL1_v1 | | | |
| Human | 42 | 43 | prey78892 |
| DVL1_v1 | | | |
| Human | 42 | 43 | prey78893 |
| DVL1_v1 | | | |
| Human | 42 | 43 | prey78894 |
| DVL1_v1 | | | |
| Human | 42 | 43 | prey78898 (prey78895; prey78896; prey78897) |
| DVL1_v1 | | | |
| Human | 42 | 43 | prey5779 (prey5778; prey32194) hprepro alpha1 (I) collagen hCOL1A1 |
| DVL1_v1 | | | |
| Human | 42 | 43 | prey2805 (DD5 HYD EDD KIAA0896; prey2806) hDD5 hEDD |
| DVL1_v1 | | | |
| Human | 42 | 43 | prey78905 (PAM; prey78911) hPAM hpeptidylglycine alpha amidating monooxygenase |
| DVL1_v1 | | | |
| Human | 42 | 43 | prey32308 (prey32307; prey32309) hproteasome (prosome, macropain) 26S subunit, non |
| DVL1_v1 | | | ATPase, 8 hPSMD8 hPSMD8 h26S proteasome subunitp31 |
| Human | 42 | 43 | prey78921 |
| DVL1_v1 | | | |
| Human | 42 | 43 | prey78922 |
| DVL1_v1 | | | |
| Human | 42 | 43 | prey68432 (CSF3R GCSFR CD114; prey68436) hCSF3R hG CSFR 1 |
| DVL1_v1 | | | |
| Human | 42 | 43 | prey78925 (prey78924) |
| DVL1_v1 | | | |
| Human | 42 | 43 | prey78928 |
| DVL1_v1 | | | |
| Human | 42 | 43 | prey78935 |
| DVL1_v1 | | | |
| Human | 42 | 43 | prey67563 (PRSC1; prey67565) hPRSC1 hlegumain |
| DVL1_v1 | | | |
| Human | 42 | 43 | prey104794 |
| DVL1_v1 | | | |
| Human | 42 | 43 | prey104797 |
| DVL1_v1 | | | |
| Human | 42 | 43 | prey104799 |
| DVL1_v1 | | | |
| Human | 42 | 43 | prey92121 (LOC162427) hhypothetical proteinXP_091549 |
| DVL1_v1 | | | |
| Human | 42 | 43 | prey104802 |
| DVL1_v1 | | | |
| Human | 42 | 43 | prey78388 (CCNI CYC1 CYI) hCCNI |
| DVL1_v1 | | | |
| Human | 42 | 43 | prey32308 (prey32307; prey32309) hproteasome (prosome, macropain) 26S subunit, non |
| DVL1_v1 | | | ATPase, 8 hPSMD8 hPSMD8 h26S proteasome subunitp31 |
| Human | 42 | 43 | prey104808 |
| DVL1_v1 | | | |
| Human | 42 | 43 | prey104810 |
| DVL1_v1 | | | |
| Human | 42 | 43 | prey104811 |
| DVL1_v1 | | | |
| Human | 42 | 43 | prey104813 |
| DVL1_v1 | | | |
| Human | 42 | 43 | prey70344 (ATP6E; prey70347) hATP6E hvacuolar H+ ATPase Esubunit |
| DVL1_v1 | | | |
| Human | 42 | 43 | prey104818 |
| DVL1_v1 | | | |
| Human | 42 | 43 | prey102423 (ZAP3) hZAP3 |
| DVL1_v1 | | | |
| Human | 42 | 43 | prey104821 |
| DVL1_v1 | | | |
| Human | 42 | 43 | prey78495 |
| DVL1_v1 | | | |
| Human | 42 | 43 | prey104823 |
| DVL1_v1 | | | |
| Human | 42 | 43 | prey102110 (SNAP23 SNAP23A SNAP23B) hSNAP23 |
| DVL1_v1 | | | |
| Human | 42 | 43 | prey104828 |
| DVL1_v1 | | | |
| Human | 42 | 43 | prey104829 |
| DVL1_v1 | | | |
| Human | 42 | 43 | prey100907 (LOC157909) hhypothetical proteinXP_088419 |
| DVL1_v1 | | | |
| Human | 42 | 43 | prey104833 |
| DVL1_v1 | | | |
| Human | 42 | 43 | prey13439 (KIAA1012; prey13442) hKIAA1012 |
| DVL1_v1 | | | |
| Human | 42 | 43 | prey104840 |
| DVL1_v1 | | | |
| Human | 42 | 43 | prey104843 |
| DVL1_v1 | | | |
| Human | 42 | 43 | prey104844 |
| DVL1_v1 | | | |
| Human | 42 | 43 | prey104845 |
| DVL1_v1 | | | |
| Human | 42 | 43 | prey68432 (CSF3R GCSFR CD114; prey68436) hCSF3R hG CSFR 1 |
| DVL1_v1 | | | |
| Human | 42 | 43 | prey104850 |
| DVL1_v1 | | | |
| Human | 42 | 43 | prey104854 |
| DVL1_v1 | | | |
| Human | 42 | 43 | prey78679 (MYNN SBBIZ1; prey78681) hMYNN hmyoneurin |
| DVL1_v1 | | | |
| Human | 42 | 43 | prey78564 (prey102151) hUnknown (protein forMGC: 27165) hUnknown (protein |
| DVL1_v1 | | | forMGC: 27428) |
| Human | 42 | 43 | prey104856 |
| DVL1_v1 | | | |
| Human | 42 | 43 | prey78440 (ADARB1 RED1 ADAR2 DRADA2 DRABA2; prey78442) hADARB1 hDRADA2c |
| DVL1_v1 | | | |
| Human | 42 | 43 | prey51725 |
| DVL1_v1 | | | |
| Human | 42 | 43 | prey104864 |
| DVL1_v1 | | | |
| Human | 42 | 43 | prey12054 (COPB2; prey12055) hCOPB2 hsubunit of coatomercomplex |
| DVL1_v1 | | | |
| Human | 42 | 43 | prey102131 (DCX DC DBCN LISX SCLH XLIS) hDCX |
| DVL1_v1 | | | |
| Human | 42 | 43 | prey104867 |
| DVL1_v1 | | | |
| Human | 42 | 43 | prey54064 (CGA HCG) hCGA |
| DVL1_v1 | | | |
| Human | 42 | 43 | prey104869 |
| DVL1_v1 | | | |
| Human | 42 | 43 | prey104870 |
| DVL1_v1 | | | |
| Human | 42 | 43 | prey104949 |
| DVL1_v1 | | | |
| Human | 42 | 43 | prey1469 (COL3A1; prey1473; prey19974; prey22635) hCOL3A1 hprepro alpha 1 type |
| DVL1_v1 | | | 3collagen hCOL3A1 |
| Human | 42 | 43 | prey50431 (CD74 DHLAG; prey50433) hCD74 |
| DVL1_v1 | | | |
| Human | 42 | 43 | prey104876 |
| DVL1_v1 | | | |
| Human | 42 | 43 | prey105012 |
| DVL1_v1 | | | |
| Human | 42 | 43 | prey102333 (LOC91378) hsimilar to hypothetical proteinDKFZp434F142 |
| DVL1_v1 | | | |
| Human | 42 | 43 | prey5779 (prey5778; prey32194) hprepro alpha1 (I) collagen hCOL1A1 |
| DVL1_v1 | | | |
| Human | 42 | 43 | prey104882 |
| DVL1_v1 | | | |
| Human | 42 | 43 | prey104886 |
| DVL1_v1 | | | |
| Human | 42 | 43 | prey93097 (CTSD CPSD) hCTSD |
| DVL1_v1 | | | |
| Human | 42 | 43 | prey104890 |
| DVL1_v1 | | | |
| Human | 42 | 43 | prey102198 (LOC148635) hhypothetical proteinXP_086260 |
| DVL1_v1 | | | |
| Human | 42 | 43 | prey8065 (CCT7 CCTH NIP7 1; prey8066) hCCT7 hCcth |
| DVL1_v1 | | | |
| Human | 42 | 43 | prey104894 |
| DVL1_v1 | | | |
| Human | 42 | 43 | prey104896 |
| DVL1_v1 | | | |
| Human | 42 | 43 | prey102355 (PTEN MMAC1 MHAM BZS; prey102359) hPTEN hMMAC1 |
| DVL1_v1 | | | |
| Human | 42 | 43 | prey104899 |
| DVL1_v1 | | | |
| Human | 42 | 43 | prey104909 |
| DVL1_v1 | | | |
| Human | 42 | 43 | prey102479 (LOC163122) hhypothetical proteinXP_103267 |
| DVL1_v1 | | | |
| Human | 42 | 43 | prey78905 (PAM; prey78911) hPAM hpeptidylglycine alpha amidating monooxygenase |
| DVL1_v1 | | | |
| Human | 42 | 43 | prey2527 (MLLT2 AF4 AF 4; prey2528) hMLLT2 hAF 4 |
| DVL1_v1 | | | |
| Human | 42 | 43 | prey92918 (LOC157934) hhypothetical proteinXP_088426 |
| DVL1_v1 | | | |
| Human | 42 | 43 | prey102273 (CD74 DHLAG) hCD74 |
| DVL1_v1 | | | |
| Human | 42 | 43 | prey104937 |
| DVL1_v1 | | | |
| Human | 42 | 43 | prey104938 |
| DVL1_v1 | | | |
| Human | 42 | 43 | prey104941 |
| DVL1_v1 | | | |
| Human | 42 | 43 | prey69469 hHMG box containing protein1 |
| DVL1_v1 | | | |
| Human | 42 | 43 | prey104944 |
| DVL1_v1 | | | |
| Human | 42 | 43 | prey56846 (TNA TN; prey56847) hTNA hTetranectin |
| DVL1_v1 | | | |
| Human | 42 | 43 | prey104947 |
| DVL1_v1 | | | |
| Human | 42 | 43 | prey104951 |
| DVL1_v1 | | | |
| Human | 42 | 43 | prey104954 |
| DVL1_v1 | | | |
| Human | 42 | 43 | prey104961 |
| DVL1_v1 | | | |
| Human | 42 | 43 | prey104962 |
| DVL1_v1 | | | |
| Human | 42 | 43 | prey104964 |
| DVL1_v1 | | | |
| Human | 42 | 43 | prey104984 |
| DVL1_v1 | | | |
| Human | 42 | 43 | prey104985 |
| DVL1_v1 | | | |
| Human | 42 | 43 | prey78515 (CENTG3 MRIP 1; prey78519) hCENTG3 hMRIP 1 |
| DVL1_v1 | | | |
| Human | 42 | 43 | prey5244 (LTBP1; prey5245) hLTBP1 hTGFB1 |
| DVL1_v1 | | | |
| Human | 42 | 43 | prey104988 |
| DVL1_v1 | | | |
| Human | 42 | 43 | prey104990 |
| DVL1_v1 | | | |
| Human | 42 | 43 | prey102493 (KIAA0746) hKIAA0746 |
| DVL1_v1 | | | |
| Human | 42 | 43 | prey104991 |
| DVL1_v1 | | | |
| Human | 42 | 43 | prey104993 |
| DVL1_v1 | | | |
| Human | 42 | 43 | prey104995 |
| DVL1_v1 | | | |
| Human | 42 | 43 | prey102159 (SMG1 KIAA0421 LIP) hSMG1 |
| DVL1_v1 | | | |
| Human | 42 | 43 | prey105000 |
| DVL1_v1 | | | |
| Human | 42 | 43 | prey105001 |
| DVL1_v1 | | | |
| Human | 42 | 43 | prey105005 |
| DVL1_v1 | | | |
| Human | 42 | 43 | prey78563 |
| DVL1_v1 | | | |
| Human | 42 | 43 | prey27857 (ERVWE1 LOC30816 SYNCYTIN HERV W HERVW; prey27858; prey27864) hERVWE1 |
| DVL1_v1 | | | henvelope proteinprecursor |
| Human | 42 | 43 | prey78469 (FLJ20199; prey78470) hFLJ20199 |
| DVL1_v1 | | | |
| Human | 42 | 43 | prey105019 |
| DVL1_v1 | | | |
| Human | 42 | 43 | prey105021 |
| DVL1_v1 | | | |
| Human | 42 | 43 | prey78379 (NEU1 NEU SIAL1; prey78382) hNEU1 hlysosomal neuraminidaseprecursor |
| DVL1_v1 | | | |
| Human | 42 | 43 | prey105022 |
| DVL1_v1 | | | |
| Human | 42 | 43 | prey105029 |
| DVL1_v1 | | | |
| Human | 42 | 43 | prey105031 |
| DVL1_v1 | | | |
| Human | 42 | 43 | prey102321 |
| DVL1_v1 | | | |
| Human | 42 | 43 | prey105032 |
| DVL1_v1 | | | |
| Human | 42 | 43 | prey102459 (DC42) hDC42 |
| DVL1_v1 | | | |
| Human | 42 | 43 | prey107650 (LOC148433) hsimilar to homeo box A7 (H. sapiens ) |
| DVL1_v1 | | | |
| Human | 42 | 43 | prey92681 |
| DVL1_v1 | | | |
| Human | 42 | 43 | prey107662 |
| DVL1_v1 | | | |
| Human | 42 | 43 | prey107665 |
| DVL1_v1 | | | |
| Human | 42 | 43 | prey107667 |
| DVL1_v1 | | | |
| Human | 42 | 43 | prey107681 (KIAA1414) hKIAA1414 |
| DVL1_v1 | | | |
| Human | 42 | 43 | prey107678 |
| DVL1_v1 | | | |
| Human | 42 | 43 | prey107692 |
| DVL1_v1 | | | |
| Human | 42 | 43 | prey107848 |
| DVL1_v1 | | | |
| Human | 42 | 43 | prey107700 |
| DVL1_v1 | | | |
| Human | 42 | 43 | prey107702 |
| DVL1_v1 | | | |
| Human | 42 | 43 | prey1080 (MADH5 JV5 1 SMAD5; prey1083) hMADH5 hSmad5 MAD likeprotein |
| DVL1_v1 | | | |
| Human | 42 | 43 | prey78388 (CCNI CYC1 CYI) hCCNI |
| DVL1_v1 | | | |
| Human | 42 | 43 | prey107740 |
| DVL1_v1 | | | |
| Human | 42 | 43 | prey107745 |
| DVL1_v1 | | | |
| Human | 42 | 43 | prey78271 (prey67898; prey67896; prey67901; prey66163; prey66165; prey66161; |
| DVL1_v1 | | | prey67897; prey66169; prey70558; prey67899; prey67900; prey74048; prey66160; |
| | | | prey66162; prey66166; prey66167; prey66168; prey100021) |
| Human | 42 | 43 | prey107747 |
| DVL1_v1 | | | |
| Human | 42 | 43 | prey95593 (ENO2 NSE) hENO2 |
| DVL1_v1 | | | |
| Human | 42 | 43 | prey107763 (LOC131110) hhypothetical proteinXP_059494 |
| DVL1_v1 | | | |
| Human | 42 | 43 | prey107767 (SLC31A1 COPT1 CTR1) hSLC31A1 |
| DVL1_v1 | | | |
| Human | 42 | 43 | prey107826 |
| DVL1_v1 | | | |
| Human | 42 | 43 | prey107782 |
| DVL1_v1 | | | |
| Human | 42 | 43 | prey107784 |
| DVL1_v1 | | | |
| Human | 42 | 43 | prey107799 |
| DVL1_v1 | | | |
| Human | 42 | 43 | prey1469 (COL3A1; prey1473; prey19974; prey22635) hCOL3A1 hprepro alpha 1 type |
| DVL1_v1 | | | 3collagen hCOL3A1 |
| Human | 42 | 43 | prey107812 |
| DVL1_v1 | | | |
| Human | 42 | 43 | prey107814 |
| DVL1_v1 | | | |
| Human | 42 | 43 | prey107816 |
| DVL1_v1 | | | |
| Human | 42 | 43 | prey107817 |
| DVL1_v1 | | | |
| Human | 42 | 43 | prey107822 |
| DVL1_v1 | | | |
| Human | 42 | 43 | prey107823 |
| DVL1_v1 | | | |
| Human | 42 | 43 | prey107828 |
| DVL1_v1 | | | |
| Human | 42 | 43 | prey107832 |
| DVL1_v1 | | | |
| Human | 42 | 43 | prey31081 (JM23; prey31083; prey31085) hJM23 |
| DVL1_v1 | | | |
| Human | 42 | 43 | prey107854 |
| DVL1_v1 | | | |
| Human | 42 | 43 | prey107855 |
| DVL1_v1 | | | |
| Human | 42 | 43 | prey107861 |
| DVL1_v1 | | | |
| Human | 42 | 43 | prey72968 (KIAA0317; prey72971) hKIAA0317 |
| DVL1_v1 | | | |
| Human | 42 | 43 | prey107865 |
| DVL1_v1 | | | |
| Human | 42 | 43 | prey107866 |
| DVL1_v1 | | | |
| Human | 42 | 43 | prey107869 |
| DVL1_v1 | | | |
| Human | 42 | 43 | prey107871 |
| DVL1_v1 | | | |
| Human | 42 | 43 | prey107875 |
| DVL1_v1 | | | |
| Human | 42 | 43 | prey107876 |
| DVL1_v1 | | | |
| Human | 42 | 43 | prey107879 |
| DVL1_v1 | | | |
| Human | 42 | 43 | prey106011 (LOC161828) hhypothetical proteinXP_091160 |
| DVL1_v1 | | | |
| Human | 42 | 43 | prey107880 |
| DVL1_v1 | | | |
| Human | 42 | 43 | prey108056 |
| DVL1_v1 | | | |
| Human | 42 | 43 | prey107884 |
| DVL1_v1 | | | |
| Human | 42 | 43 | prey107891 |
| DVL1_v1 | | | |
| Human | 42 | 43 | prey107893 |
| DVL1_v1 | | | |
| Human | 42 | 43 | prey107894 |
| DVL1_v1 | | | |
| Human | 42 | 43 | prey70344 (ATP6E; prey70347) hATP6E hvacuolar H+ ATPase Esubunit |
| DVL1_v1 | | | |
| Human | 42 | 43 | prey107907 |
| DVL1_v1 | | | |
| Human | 42 | 43 | prey107910 |
| DVL1_v1 | | | |
| Human | 42 | 43 | prey107931 |
| DVL1_v1 | | | |
| Human | 42 | 43 | prey5779 (prey5778; prey32194) hprepro alpha1 (I) collagen hCOL1A1 |
| DVL1_v1 | | | |
| Human | 42 | 43 | prey107934 |
| DVL1_v1 | | | |
| Human | 42 | 43 | prey107942 |
| DVL1_v1 | | | |
| Human | 42 | 43 | prey107946 |
| DVL1_v1 | | | |
| Human | 42 | 43 | prey107952 |
| DVL1_v1 | | | |
| Human | 42 | 43 | prey107953 |
| DVL1_v1 | | | |
| Human | 42 | 43 | prey107956 |
| DVL1_v1 | | | |
| Human | 42 | 43 | prey107959 |
| DVL1_v1 | | | |
| Human | 42 | 43 | prey107963 |
| DVL1_v1 | | | |
| Human | 42 | 43 | prey67271 (STAT5B STAT5; prey67274) hSTAT5B hsignal transducer and activator of |
| DVL1_v1 | | | transcriptionStat5B |
| Human | 42 | 43 | prey107968 |
| DVL1_v1 | | | |
| Human | 42 | 43 | prey107969 |
| DVL1_v1 | | | |
| Human | 42 | 43 | prey107971 |
| DVL1_v1 | | | |
| Human | 42 | 43 | prey107976 |
| DVL1_v1 | | | |
| Human | 42 | 43 | prey107981 |
| DVL1_v1 | | | |
| Human | 42 | 43 | prey107987 |
| DVL1_v1 | | | |
| Human | 42 | 43 | prey107993 |
| DVL1_v1 | | | |
| Human | 42 | 43 | prey108000 |
| DVL1_v1 | | | |
| Human | 42 | 43 | prey108007 (AKR1C1 DDH MBAB DDH1 DD1 2 ALPHA HSD HBAB HAKRC 20 ALPHA HSD H 37) |
| DVL1_v1 | | | hAKR1C1 |
| Human | 42 | 43 | prey108017 (KIAA0339) hKIAA0339 |
| DVL1_v1 | | | |
| Human | 42 | 43 | prey108018 |
| DVL1_v1 | | | |
| Human | 42 | 43 | prey108019 |
| DVL1_v1 | | | |
| Human | 42 | 43 | prey108020 |
| DVL1_v1 | | | |
| Human | 42 | 43 | prey108025 |
| DVL1_v1 | | | |
| Human | 42 | 43 | prey108027 |
| DVL1_v1 | | | |
| Human | 42 | 43 | prey108034 |
| DVL1_v1 | | | |
| Human | 42 | 43 | prey108035 |
| DVL1_v1 | | | |
| Human | 42 | 43 | prey95798 (AKAP9 CG NAP YOTIAO AKAP120 AKAP350 AKAP450 KIAA0803) hAKAP9 |
| DVL1_v1 | | | |
| Human | 42 | 43 | prey108040 |
| DVL1_v1 | | | |
| Human | 42 | 43 | prey108041 |
| DVL1_v1 | | | |
| Human | 42 | 43 | prey108042 |
| DVL1_v1 | | | |
| Human | 42 | 43 | prey56754 (PHKA2 PHK PYK PYKL XLG XLG2; prey56756) hPHKA2 hPHKLA |
| DVL1_v1 | | | |
| Human | 42 | 43 | prey108048 |
| DVL1_v1 | | | |
| Human | 42 | 43 | prey108049 |
| DVL1_v1 | | | |
| Human | 42 | 43 | prey108051 |
| DVL1_v1 | | | |
| Human | 42 | 43 | prey108052 |
| DVL1_v1 | | | |
| Human | 42 | 43 | prey3794 hhypothetical proteinFLJ10461 |
| DVL1_v1 | | | |
| Human | 42 | 43 | prey108059 |
| DVL1_v1 | | | |
| Human | 42 | 43 | prey108062 |
| DVL1_v1 | | | |
| Human | 42 | 43 | prey108066 |
| DVL1_v1 | | | |
| Human | 42 | 43 | prey108070 |
| DVL1_v1 | | | |
| Human | 42 | 43 | prey108072 |
| DVL1_v1 | | | |
| Human | 42 | 43 | prey108073 putative homolog of prey010635 |
| DVL1_v1 | | | |
| Human | 42 | 43 | prey108074 |
| DVL1_v1 | | | |
| Human | 42 | 43 | prey108075 |
| DVL1_v1 | | | |
| Human | 42 | 43 | prey108076 |
| DVL1_v1 | | | |
| Human | 42 | 43 | prey64719 (hMAST205; prey64715) hhMAST205 |
| DVL1_v1 | | | |
| Human | 42 | 43 | prey108082 |
| DVL1_v1 | | | |
| Human | 42 | 43 | prey108083 |
| DVL1_v1 | | | |
| Human | 42 | 43 | prey108088 (SEPN1 SelN RSMD1) hSEPN1 |
| DVL1_v1 | | | |
| Human | 42 | 43 | prey108093 |
| DVL1_v1 | | | |
| Human | 42 | 43 | prey108097 |
| DVL1_v1 | | | |
| Human | 42 | 43 | prey108102 |
| DVL1_v1 | | | |
| Human | 42 | 43 | prey108100 |
| DVL1_v1 | | | |
| Human | 42 | 43 | prey108105 |
| DVL1_v1 | | | |
| Human | 42 | 43 | prey108107 |
| DVL1_v1 | | | |
| Human | 42 | 43 | prey108108 |
| DVL1_v1 | | | |
| Human | 42 | 43 | prey51316 (COL5A2) hCOL5A2 |
| DVL1_v1 | | | |
| Human | 42 | 43 | prey108109 |
| DVL1_v1 | | | |
| Human | 42 | 43 | prey108110 |
| DVL1_v1 | | | |
| Human | 42 | 43 | prey108112 |
| DVL1_v1 | | | |
| Human | 42 | 43 | prey108115 |
| DVL1_v1 | | | |
| Human | 42 | 43 | prey108116 |
| DVL1_v1 | | | |
| Human | 42 | 43 | prey108117 |
| DVL1_v1 | | | |
| Human | 42 | 43 | prey99362 (GCN1L1 GCN1L KIAA0219) hGCN1L1 |
| DVL1_v1 | | | |
| Human | 42 | 43 | prey108121 |
| DVL1_v1 | | | |
| Human | 42 | 43 | prey108122 |
| DVL1_v1 | | | |
| Human | 42 | 43 | prey108123 |
| DVL1_v1 | | | |
| Human | 42 | 43 | prey108124 |
| DVL1_v1 | | | |
| Human | 42 | 43 | prey56846 (TNA TN; prey56847) hTNA hTetranectin |
| DVL1_v1 | | | |
| Human | 42 | 43 | prey108133 |
| DVL1_v1 | | | |
| Human | 42 | 43 | prey108134 |
| DVL1_v1 | | | |
| Human | 42 | 43 | prey92918 (LOC157934) hhypothetical proteinXP_088426 |
| DVL1_v1 | | | |
| Human | 42 | 43 | prey108139 |
| DVL1_v1 | | | |
| Human | 42 | 43 | prey108142 |
| DVL1_v1 | | | |
| Human | 42 | 43 | prey108143 |
| DVL1_v1 | | | |
| Human | 42 | 43 | prey108157 |
| DVL1_v1 | | | |
| Human | 42 | 43 | prey108159 |
| DVL1_v1 | | | |
| Human | 42 | 43 | prey108160 |
| DVL1_v1 | | | |
| Human | 44 | 45 | prey56846 (TNA TN; prey56847) hTNA hTetranectin |
| DVL2_v1 | | | |
| Human | 44 | 45 | prey78738 |
| DVL2_v1 | | | |
| Human | 44 | 45 | prey78741 |
| DVL2_v1 | | | |
| Human | 44 | 45 | prey78742 |
| DVL2 _v1 | | | |
| Human | 44 | 45 | prey78744 |
| DVL2_v1 | | | |
| Human | 44 | 45 | prey3062 (ARRB1 ARR1) hARRB1 |
| DVL2_v1 | | | |
| Human | 44 | 45 | prey37079 (C4orf5 CS 1; prey37081) hC4orf5 |
| DVL2_v1 | | | |
| Human | 44 | 45 | prey78756 |
| DVL2_v1 | | | |
| Human | 44 | 45 | prey78757 |
| DVL2_v1 | | | |
| Human | 44 | 45 | prey78758 |
| DVL2_v1 | | | |
| Human | 44 | 45 | prey78599 (LOC55901; prey78601) hLOC55901 hTMTSP |
| DVL2_v1 | | | |
| Human | 44 | 45 | prey78760 |
| DVL2_v1 | | | |
| Human | 44 | 45 | prey78761 |
| DVL2_v1 | | | |
| Human | 44 | 45 | prey78763 |
| DVL2_v1 | | | |
| Human | 44 | 45 | prey78764 |
| DVL2_v1 | | | |
| Human | 44 | 45 | prey78767 |
| DVL2_v1 | | | |
| Human | 44 | 45 | prey32308 (prey32307; prey32309) hproteasome (prosome, macropain) 26S subunit, non |
| DVL2_v1 | | | ATPase,8 hPSMD8 hPSMD8 h26S proteasome subunitp31 |
| Human | 44 | 45 | prey78770 |
| DVL2_v1 | | | |
| Human | 44 | 45 | prey78772 |
| DVL2_v1 | | | |
| Human | 44 | 45 | prey78773 |
| DVL2_v1 | | | |
| Human | 44 | 45 | prey323 (CSH1 CSMT CSA PL; prey324; prey325) hCSH1 |
| DVL2_v1 | | | |
| Human | 44 | 45 | prey78778 (PSG5; prey78779) hPSG5 |
| DVL2_v1 | | | |
| Human | 44 | 45 | prey78782 |
| DVL2_v1 | | | |
| Human | 44 | 45 | prey78783 |
| DVL2_v1 | | | |
| Human | 44 | 45 | prey78785 |
| DVL2_v1 | | | |
| Human | 44 | 45 | prey78790 |
| DVL2_v1 | | | |
| Human | 44 | 45 | prey78486 (CD74 DHLAG) hCD74 |
| DVL2_v1 | | | |
| Human | 44 | 45 | prey78791 |
| DVL2_v1 | | | |
| Human | 44 | 45 | prey78798 |
| DVL2_v1 | | | |
| Human | 44 | 45 | prey78807 |
| DVL2_v1 | | | |
| Human | 44 | 45 | prey78811 (C20orf162 dJ337O18.5; prey78813) hC20orf162 hUnknown (protein |
| DVL2_v1 | | | forMGC: 2467) |
| Human | 44 | 45 | prey78823 |
| DVL2_v1 | | | |
| Human | 44 | 45 | prey78824 |
| DVL2_v1 | | | |
| Human | 44 | 45 | prey78825 (EPB41L4 NBL4; prey78826) hEPB41L4 hhNBL4 |
| DVL2_v1 | | | |
| Human | 44 | 45 | prey78828 |
| DVL2_v1 | | | |
| Human | 44 | 45 | prey78830 |
| DVL2_v1 | | | |
| Human | 44 | 45 | prey78838 |
| DVL2_v1 | | | |
| Human | 44 | 45 | prey78842 |
| DVL2_v1 | | | |
| Human | 44 | 45 | prey78843 |
| DVL2_v1 | | | |
| Human | 44 | 45 | prey78846 |
| DVL2_v1 | | | |
| Human | 44 | 45 | prey51748 (ARRB2 ARR2) hARRB2 |
| DVL2_v1 | | | |
| Human | 44 | 45 | prey82357 |
| DVL2_v1 | | | |
| Human | 44 | 45 | prey45630 (IGFBP5 IBP5; prey45632) hIGFBP5 |
| DVL2_v1 | | | |
| Human | 44 | 45 | prey82359 |
| DVL2_v1 | | | |
| Human | 44 | 45 | prey77557 (KIAA1095; prey4098) hKIAA1095 |
| DVL2_v1 | | | |
| Human | 44 | 45 | prey82363 |
| DVL2_v1 | | | |
| Human | 44 | 45 | prey32308 (prey32307; prey32309) hproteasome (prosome, macropain) 26S subunit, non |
| DVL2_v1 | | | ATPase,8 hPSMD8 hPSMD8 h26S proteasome subunitp31 |
| Human | 44 | 45 | prey56846 (TNA TN; prey56847) hTNA hTetranectin |
| DVL2_v1 | | | |
| Human | 44 | 45 | prey82378 (prey82377) |
| DVL2_v1 | | | |
| Human | 44 | 45 | prey82384 |
| DVL2_v1 | | | |
| Human | 44 | 45 | prey82387 |
| DVL2_v1 | | | |
| Human | 44 | 45 | prey82395 |
| DVL2_v1 | | | |
| Human | 44 | 45 | prey45854 (FLII FLI; prey45856) hFLII |
| DVL2_v1 | | | |
| Human | 44 | 45 | prey82400 putative homolog of prey082399 |
| DVL2_v1 | | | |
| Human | 44 | 45 | prey29055 (TCIRG1 TIRC7 OC 116 OC 116 kDa ATP6N1C OC116 OPTB1; prey29057) hTCIRG1 |
| DVL2_v1 | | | hTIRC7 |
| Human | 44 | 45 | prey82412 |
| DVL2_v1 | | | |
| Human | 44 | 45 | prey82423 (prey82422) |
| DVL2_v1 | | | |
| Human | 44 | 45 | prey82425 |
| DVL2_v1 | | | |
| Human | 44 | 45 | prey92611 |
| DVL2_v1 | | | |
| Human | 44 | 45 | prey92612 |
| DVL2_v1 | | | |
| Human | 44 | 45 | prey92615 |
| DVL2_v1 | | | |
| Human | 44 | 45 | prey3341 (SAFB HET; prey3342) hSAFB hHET |
| DVL2_v1 | | | |
| Human | 44 | 45 | prey92631 |
| DVL2_v1 | | | |
| Human | 44 | 45 | prey92641 |
| DVL2_v1 | | | |
| Human | 44 | 45 | prey92646 |
| DVL2_v1 | | | |
| Human | 44 | 45 | prey93191 |
| DVL2_v1 | | | |
| Human | 44 | 45 | prey92821 |
| DVL2_v1 | | | |
| Human | 44 | 45 | prey93166 |
| DVL2_v1 | | | |
| Human | 44 | 45 | prey48610 (FN1 CIG FINC FN) hFN1 |
| DVL2_v1 | | | |
| Human | 44 | 45 | prey78599 (LOC55901; prey78601) hLOC55901 hTMTSP |
| DVL2_v1 | | | |
| Human | 44 | 45 | prey92693 |
| DVL2_v1 | | | |
| Human | 44 | 45 | prey92698 |
| DVL2_v1 | | | |
| Human | 44 | 45 | prey92714 |
| DVL2_v1 | | | |
| Human | 44 | 45 | prey92741 |
| DVL2_v1 | | | |
| Human | 44 | 45 | prey92744 (prey92742) |
| DVL2_v1 | | | |
| Human | 44 | 45 | prey92745 |
| DVL2_v1 | | | |
| Human | 44 | 45 | prey92747 |
| DVL2_v1 | | | |
| Human | 44 | 45 | prey92938 |
| DVL2_v1 | | | |
| Human | 44 | 45 | prey92796 |
| DVL2_v1 | | | |
| Human | 44 | 45 | prey92805 |
| DVL2_v1 | | | |
| Human | 44 | 45 | prey92806 |
| DVL2_v1 | | | |
| Human | 44 | 45 | prey92808 |
| DVL2_v1 | | | |
| Human | 44 | 45 | prey92816 (prey92814; prey92815) |
| DVL2_v1 | | | |
| Human | 44 | 45 | prey92825 (FLJ10852 FLJ10941; prey92827) hFLJ10852 |
| DVL2_v1 | | | |
| Human | 44 | 45 | prey92845 (prey92844) |
| DVL2_v1 | | | |
| Human | 44 | 45 | prey92860 |
| DVL2_v1 | | | |
| Human | 44 | 45 | prey92861 |
| DVL2_v1 | | | |
| Human | 44 | 45 | prey92878 (prey92877) |
| DVL2_v1 | | | |
| Human | 44 | 45 | prey92886 |
| DVL2_v1 | | | |
| Human | 44 | 45 | prey92905 |
| DVL2_v1 | | | |
| Human | 44 | 45 | prey92907 |
| DVL2_v1 | | | |
| Human | 44 | 45 | prey92908 (MGC5627; prey92909) hMGC5627 hUnknown (protein forMGC: 5627) |
| DVL2_v1 | | | |
| Human | 44 | 45 | prey56687 hcysteineprotease |
| DVL2_v1 | | | |
| Human | 44 | 45 | prey92929 (prey92928) |
| DVL2_v1 | | | |
| Human | 44 | 45 | prey92933 (prey92931; prey92932) |
| DVL2_v1 | | | |
| Human | 44 | 45 | prey92941 |
| DVL2_v1 | | | |
| Human | 44 | 45 | prey92954 |
| DVL2_v1 | | | |
| Human | 44 | 45 | prey92956 |
| DVL2_v1 | | | |
| Human | 44 | 45 | prey92957 |
| DVL2_v1 | | | |
| Human | 44 | 45 | prey92959 |
| DVL2_v1 | | | |
| Human | 44 | 45 | prey93006 (prey93005) |
| DVL2_v1 | | | |
| Human | 44 | 45 | prey93089 (prey93088) |
| DVL2_v1 | | | |
| Human | 44 | 45 | prey92962 |
| DVL2_v1 | | | |
| Human | 44 | 45 | prey92971 |
| DVL2_v1 | | | |
| Human | 44 | 45 | prey22857 (ATP2B4 ATP2B2 PMCA4; prey22859) hATP2B4 hcalcium ATPase |
| DVL2_v1 | | | (hPMCA4)precursor |
| Human | 44 | 45 | prey92975 |
| DVL2_v1 | | | |
| Human | 44 | 45 | prey92976 |
| DVL2_v1 | | | |
| Human | 44 | 45 | prey92977 |
| DVL2_v1 | | | |
| Human | 44 | 45 | prey92979 |
| DVL2_v1 | | | |
| Human | 44 | 45 | prey92981 |
| DVL2_v1 | | | |
| Human | 44 | 45 | prey92989 |
| DVL2_v1 | | | |
| Human | 44 | 45 | prey92990 |
| DVL2_v1 | | | |
| Human | 44 | 45 | prey93095 |
| DVL2_v1 | | | |
| Human | 44 | 45 | prey92992 |
| DVL2_v1 | | | |
| Human | 44 | 45 | prey92993 |
| DVL2_v1 | | | |
| Human | 44 | 45 | prey92996 (ASKL1) hASKL1 |
| DVL2_v1 | | | |
| Human | 44 | 45 | prey93001 |
| DVL2_v1 | | | |
| Human | 44 | 45 | prey93002 |
| DVL2_v1 | | | |
| Human | 44 | 45 | prey93003 |
| DVL2_v1 | | | |
| Human | 44 | 45 | prey93012 |
| DVL2_v1 | | | |
| Human | 44 | 45 | prey72968 (KIAA0317; prey72971) hKIAA0317 |
| DVL2_v1 | | | |
| Human | 44 | 45 | prey32308 (prey32307; prey32309) hproteasome (prosome, macropain) 26S subunit, non |
| DVL2_v1 | | | ATPase, 8 hPSMD8 hPSMD8 h26S proteasome subunitp31 |
| Human | 44 | 45 | prey78905 (PAM; prey78911) hPAM hpeptidylglycine alpha amidating monooxygenase |
| DVL2_v1 | | | |
| Human | 44 | 45 | prey93040 (prey84360; prey84361; prey93036; prey93037; prey93038; prey93039) |
| DVL2_v1 | | | |
| Human | 44 | 45 | prey93042 |
| DVL2_v1 | | | |
| Human | 44 | 45 | prey93049 (prey93046; prey93047; prey93048) |
| DVL2_v1 | | | |
| Human | 44 | 45 | prey93052 |
| DVL2_v1 | | | |
| Human | 44 | 45 | prey93055 |
| DVL2_v1 | | | |
| Human | 44 | 45 | prey93057 |
| DVL2_v1 | | | |
| Human | 44 | 45 | prey93069 |
| DVL2_v1 | | | |
| Human | 44 | 45 | prey93071 |
| DVL2_v1 | | | |
| Human | 44 | 45 | prey93076 |
| DVL2_v1 | | | |
| Human | 44 | 45 | prey93082 (prey93080; prey93081) |
| DVL2_v1 | | | |
| Human | 44 | 45 | prey93091 (EIF4G3 EIF4GII; prey93093) hEIF4G3 heIF4GII |
| DVL2_v1 | | | |
| Human | 44 | 45 | prey93100 |
| DVL2_v1 | | | |
| Human | 44 | 45 | prey93103 |
| DVL2_v1 | | | |
| Human | 44 | 45 | prey93104 (prey84462) |
| DVL2_v1 | | | |
| Human | 44 | 45 | prey93105 |
| DVL2_v1 | | | |
| Human | 44 | 45 | prey93118 |
| DVL2_v1 | | | |
| Human | 44 | 45 | prey93122 |
| DVL2_v1 | | | |
| Human | 44 | 45 | prey37079 (C4orf5 CS 1; prey37081) hC4orf5 |
| DVL2_v1 | | | |
| Human | 44 | 45 | prey93127 |
| DVL2_v1 | | | |
| Human | 44 | 45 | prey93134 |
| DVL2_v1 | | | |
| Human | 44 | 45 | prey93137 (prey93135; prey93136) |
| DVL2_v1 | | | |
| Human | 44 | 45 | prey93141 (prey93138; prey93139; prey93140) |
| DVL2_v1 | | | |
| Human | 44 | 45 | prey93144 (prey93142; prey93143) |
| DVL2_v1 | | | |
| Human | 44 | 45 | prey93145 |
| DVL2_v1 | | | |
| Human | 44 | 45 | prey93151 |
| DVL2_v1 | | | |
| Human | 44 | 45 | prey93154 (prey93153) |
| DVL2_v1 | | | |
| Human | 44 | 45 | prey93155 |
| DVL2_v1 | | | |
| Human | 44 | 45 | prey93159 |
| DVL2_v1 | | | |
| Human | 44 | 45 | prey93160 (ARRB1 ARR1) hARRB1 |
| DVL2_v1 | | | |
| Human | 44 | 45 | prey93161 |
| DVL2_v1 | | | |
| Human | 44 | 45 | prey93163 |
| DVL2_v1 | | | |
| Human | 44 | 45 | prey93169 |
| DVL2_v1 | | | |
| Human | 44 | 45 | prey93176 |
| DVL2_v1 | | | |
| Human | 44 | 45 | prey56846 (TNA TN; prey56847) hTNA hTetranectin |
| DVL2_v1 | | | |
| Human | 44 | 45 | prey93186 (prey93179; prey93180; prey93181; prey93182; prey93183; prey93184; |
| DVL2_v1 | | | prey93185) |
| Human | 44 | 45 | prey84370 (MORF KIAA0383; prey84373) hMORF hhistone acetyltransferase MORFbeta |
| DVL2_v1 | | | |
| Human | 44 | 45 | prey93188 |
| DVL2_v1 | | | |
| Human | 44 | 45 | prey93189 |
| DVL2_v1 | | | |
| Human | 44 | 45 | prey93193 |
| DVL2_v1 | | | |
| Human | 44 | 45 | prey93194 |
| DVL2_v1 | | | |
| Human | 44 | 45 | prey93197 |
| DVL2_v1 | | | |
| Human | 44 | 45 | prey93201 |
| DVL2_v1 | | | |
| Human | 44 | 45 | prey93202 |
| DVL2_v1 | | | |
| Human | 44 | 45 | prey93207 (prey93205; prey93206) |
| DVL2_v1 | | | |
| Human | 44 | 45 | prey93208 |
| DVL2_v1 | | | |
| Human | 44 | 45 | prey93214 |
| DVL2_v1 | | | |
| Human | 44 | 45 | prey93217 |
| DVL2_v1 | | | |
| Human | 46 | 47 | prey78527 |
| DVL3_v1 | | | |
| Human | 46 | 47 | prey78698 |
| DVL3_v1 | | | |
| Human | 46 | 47 | prey78492 |
| DVL3_v1 | | | |
| Human | 46 | 47 | prey78494 |
| DVL3_v1 | | | |
| Human | 46 | 47 | prey78499 |
| DVL3_v1 | | | |
| Human | 46 | 47 | prey78501 |
| DVL3_v1 | | | |
| Human | 46 | 47 | prey78736 |
| DVL3_v1 | | | |
| Human | 46 | 47 | prey70344 (ATP6E; prey70347) hATP6E hvacuolar H+ ATPase Esubunit |
| DVL3_v1 | | | |
| Human | 46 | 47 | prey78572 |
| DVL3_v1 | | | |
| Human | 46 | 47 | prey78577 |
| DVL3_v1 | | | |
| Human | 46 | 47 | prey78599 (LOC55901; prey78601) hLOC55901 hTMTSP |
| DVL3_v1 | | | |
| Human | 46 | 47 | prey78607 (LOC51339; prey78609) hLOC51339 hheptacellular carcinoma novel gene |
| DVL3_v1 | | | 3protein |
| Human | 46 | 47 | prey56846 (TNA TN; prey56847) hTNA hTetranectin |
| DVL3_v1 | | | |
| Human | 46 | 47 | prey78627 (prey78625; prey78626) |
| DVL3_v1 | | | |
| Human | 46 | 47 | prey78635 (MCP CD46; prey78637) hMCP hmembrane cofactorproteinCD46MCP |
| DVL3_v1 | | | |
| Human | 46 | 47 | prey78644 |
| DVL3_v1 | | | |
| Human | 46 | 47 | prey32308 (prey32307; prey32309) hproteasome (prosome, macropain) 26S subunit, non |
| DVL3_v1 | | | ATPase, 8 hPSMD8 hPSMD8 h26S proteasome subunitp31 |
| Human | 46 | 47 | prey78679 (MYNN SBBIZ1; prey78681) hMYNN hmyoneurin |
| DVL3_v1 | | | |
| Human | 46 | 47 | prey78662 |
| DVL3_v1 | | | |
| Human | 46 | 47 | prey78664 |
| DVL3_v1 | | | |
| Human | 46 | 47 | prey78669 |
| DVL3_v1 | | | |
| Human | 46 | 47 | prey78672 |
| DVL3_v1 | | | |
| Human | 46 | 47 | prey78674 |
| DVL3_v1 | | | |
| Human | 46 | 47 | prey78675 |
| DVL3_v1 | | | |
| Human | 46 | 47 | prey69554 (KIAA0746) hKIAA0746 |
| DVL3_v1 | | | |
| Human | 46 | 47 | prey2530 harrestin, beta1 |
| DVL3_v1 | | | |
| Human | 46 | 47 | prey78687 |
| DVL3_v1 | | | |
| Human | 46 | 47 | prey78693 |
| DVL3_v1 | | | |
| Human | 46 | 47 | prey78702 (prey78699; prey78700; prey78701) |
| DVL3_v1 | | | |
| Human | 46 | 47 | prey78705 |
| DVL3_v1 | | | |
| Human | 46 | 47 | prey78706 |
| DVL3_v1 | | | |
| Human | 46 | 47 | prey78709 |
| DVL3_v1 | | | |
| Human | 46 | 47 | prey78711 (VEGFB VRF VEGFL; prey78714) hVEGFB hVEGF B |
| DVL3_v1 | | | |
| Human | 46 | 47 | prey78718 |
| DVL3_v1 | | | |
| Human | 46 | 47 | prey78486 (CD74 DHLAG) hCD74 |
| DVL3_v1 | | | |
| Human | 46 | 47 | prey78723 |
| DVL3_v1 | | | |
| Human | 46 | 47 | prey78724 (KIAA1509) hKIAA1509 |
| DVL3_v1 | | | |
| Human | 46 | 47 | prey84247 |
| DVL3_v1 | | | |
| Human | 46 | 47 | prey84249 (LOC128486) hsimilar to dJ881L22.2 (novel protein) (H. sapiens ) |
| DVL3_v1 | | | |
| Human | 46 | 47 | prey84253 |
| DVL3_v1 | | | |
| Human | 46 | 47 | prey70344 (ATP6E; prey70347) hATP6E hvacuolar H+ ATPase Esubunit |
| DVL3_v1 | | | |
| Human | 46 | 47 | prey84262 |
| DVL3_v1 | | | |
| Human | 46 | 47 | prey84427 |
| DVL3_v1 | | | |
| Human | 46 | 47 | prey84267 |
| DVL3_v1 | | | |
| Human | 46 | 47 | prey84271 |
| DVL3_v1 | | | |
| Human | 46 | 47 | prey84276 (LOC126100) hsimilar to zinc finger protein 14 (KOX 6) GIOT 4 for |
| DVL3_v1 | | | gonadotropin inducible transcription repressor 4 (H. sapiens ) |
| Human | 46 | 47 | prey84278 |
| DVL3_v1 | | | |
| Human | 46 | 47 | prey84280 |
| DVL3_v1 | | | |
| Human | 46 | 47 | prey84285 (NXN FLJ12614; prey84289) hNXN |
| DVL3_v1 | | | |
| Human | 46 | 47 | prey84298 |
| DVL3_v1 | | | |
| Human | 46 | 47 | prey84299 |
| DVL3_v1 | | | |
| Human | 46 | 47 | prey84304 |
| DVL3_v1 | | | |
| Human | 46 | 47 | prey9012 (NIN KIAA1565; prey9015) hNIN hninein isotype2 |
| DVL3_v1 | | | |
| Human | 46 | 47 | prey84310 |
| DVL3_v1 | | | |
| Human | 46 | 47 | prey84412 |
| DVL3_v1 | | | |
| Human | 46 | 47 | prey84314 |
| DVL3_v1 | | | |
| Human | 46 | 47 | prey84315 |
| DVL3_v1 | | | |
| Human | 46 | 47 | prey84321 |
| DVL3_v1 | | | |
| Human | 46 | 47 | prey84323 |
| DVL3_v1 | | | |
| Human | 46 | 47 | prey56846 (TNA TN; prey56847) hTNA hTetranectin |
| DVL3_v1 | | | |
| Human | 46 | 47 | prey84327 (prey84325; prey84326) |
| DVL3_v1 | | | |
| Human | 46 | 47 | prey84334 |
| DVL3_v1 | | | |
| Human | 46 | 47 | prey84337 |
| DVL3_v1 | | | |
| Human | 46 | 47 | prey84338 |
| DVL3_v1 | | | |
| Human | 46 | 47 | prey84344 (GTT1) hGTT1 |
| DVL3_v1 | | | |
| Human | 46 | 47 | prey84354 |
| DVL3_v1 | | | |
| Human | 46 | 47 | prey84355 |
| DVL3_v1 | | | |
| Human | 46 | 47 | prey84356 |
| DVL3_v1 | | | |
| Human | 46 | 47 | prey93040 (prey84360; prey84361; prey93036; prey93037; prey93038; prey93039) |
| DVL3_v1 | | | |
| Human | 46 | 47 | prey84362 (MRPL13 L13 RPL13; prey84365) hMRPL13 |
| DVL3_v1 | | | |
| Human | 46 | 47 | prey84369 (prey84368) |
| DVL3_v1 | | | |
| Human | 46 | 47 | prey84370 (MORF KIAA0383; prey84373) hMORF hhistone acetyltransferase MORFbeta |
| DVL3_v1 | | | |
| Human | 46 | 47 | prey84378 |
| DVL3_v1 | | | |
| Human | 46 | 47 | prey84380 |
| DVL3_v1 | | | |
| Human | 46 | 47 | prey51316 (COL5A2) hCOL5A2 |
| DVL3_v1 | | | |
| Human | 46 | 47 | prey84382 |
| DVL3_v1 | | | |
| Human | 46 | 47 | prey84385 |
| DVL3_v1 | | | |
| Human | 46 | 47 | prey84395 |
| DVL3_v1 | | | |
| Human | 46 | 47 | prey84408 |
| DVL3_v1 | | | |
| Human | 46 | 47 | prey84409 |
| DVL3_v1 | | | |
| Human | 46 | 47 | prey84414 |
| DVL3_v1 | | | |
| Human | 46 | 47 | prey84482 (prey84480; prey84481) |
| DVL3_v1 | | | |
| Human | 46 | 47 | prey84416 |
| DVL3_v1 | | | |
| Human | 46 | 47 | prey84417 |
| DVL3_v1 | | | |
| Human | 46 | 47 | prey84425 |
| DVL3_v1 | | | |
| Human | 46 | 47 | prey84428 |
| DVL3_v1 | | | |
| Human | 46 | 47 | prey84429 |
| DVL3_v1 | | | |
| Human | 46 | 47 | prey84430 |
| DVL3_v1 | | | |
| Human | 46 | 47 | prey84431 |
| DVL3_v1 | | | |
| Human | 46 | 47 | prey84432 |
| DVL3_v1 | | | |
| Human | 46 | 47 | prey84433 |
| DVL3_v1 | | | |
| Human | 46 | 47 | prey84439 |
| DVL3_v1 | | | |
| Human | 46 | 47 | prey84442 |
| DVL3_v1 | | | |
| Human | 46 | 47 | prey84447 (prey84445; prey84446) |
| DVL3_v1 | | | |
| Human | 46 | 47 | prey29055 (TCIRG1 TIRC7 OC 116 OC 116 kDa ATP6N1C OC116 OPTB1; prey29057) hTCIRG1 |
| DVL3_v1 | | | hTIRC7 |
| Human | 46 | 47 | prey84460 |
| DVL3_v1 | | | |
| Human | 46 | 47 | prey84461 |
| DVL3_v1 | | | |
| Human | 46 | 47 | prey93104 (prey84462) |
| DVL3_v1 | | | |
| Human | 46 | 47 | prey84464 (prey84463) |
| DVL3_v1 | | | |
| Human | 46 | 47 | prey84466 |
| DVL3_v1 | | | |
| Human | 46 | 47 | prey84470 |
| DVL3_v1 | | | |
| Human | 46 | 47 | prey84471 |
| DVL3_v1 | | | |
| Human | 46 | 47 | prey84491 |
| DVL3_v1 | | | |
| Human | 46 | 47 | prey84492 |
| DVL3_v1 | | | |
| Human | 46 | 47 | prey72968 (KIAA0317; prey72971) hKIAA0317 |
| DVL3_v1 | | | |
| Human | 46 | 47 | prey84494 |
| DVL3_v1 | | | |
| Human | 46 | 47 | prey84496 |
| DVL3_v1 | | | |
| Human | 46 | 47 | prey100887 |
| DVL3_v1 | | | |
| Human | 46 | 47 | prey100896 |
| DVL3_v1 | | | |
| Human | 46 | 47 | prey100897 |
| DVL3_v1 | | | |
| Human | 46 | 47 | prey100898 |
| DVL3_v1 | | | |
| Human | 46 | 47 | prey100899 |
| DVL3_v1 | | | |
| Human | 46 | 47 | prey78825 (EPB41L4 NBL4; prey78826) hEPB41L4 hhNBL4 |
| DVL3_v1 | | | |
| Human | 46 | 47 | prey100901 (KIAA1095) hKIAA1095 |
| DVL3_v1 | | | |
| Human | 46 | 47 | prey56846 (TNA TN; prey56847) hTNA hTetranectin |
| DVL3_v1 | | | |
| Human | 46 | 47 | prey32308 (prey32307; prey32309) hproteasome (prosome, macropain) 26S subunit, non |
| DVL3_v1 | | | ATPase, 8 hPSMD8 hPSMD8 h26S proteasome subunitp31 |
| Human | 46 | 47 | prey2805 (DD5 HYD EDD KIAA0896; prey2806) hDD5 hEDD |
| DVL3_v1 | | | |
| Human | 46 | 47 | prey100907 (LOC157909) hhypothetical proteinXP_088419 |
| DVL3_v1 | | | |
| Human | 46 | 47 | prey100910 (prey100909) |
| DVL3_v1 | | | |
| Human | 46 | 47 | prey100911 |
| DVL3_v1 | | | |
| Human | 46 | 47 | prey100912 |
| DVL3_v1 | | | |
| Human | 46 | 47 | prey95593 (ENO2 NSE) hENO2 |
| DVL3_v1 | | | |
| Human | 46 | 47 | prey7155 (CSH2 CSB) hCSH2 |
| DVL3_v1 | | | |
| Human | 46 | 47 | prey78905 (PAM; prey78911) hPAM hpeptidylglycine alpha amidating monooxygenase |
| DVL3_v1 | | | |
| Human | 46 | 47 | prey100918 (PKP4 P0071) hPKP4 |
| DVL3_v1 | | | |
| Human | 46 | 47 | prey92918 (LOC157934) hhypothetical proteinXP_088426 |
| DVL3_v1 | | | |
| Human | 46 | 47 | prey49397 (PAPPA IGFBP 4ase PAPA) hPAPPA |
| DVL3_v1 | | | |

TABLE 3

SID sequences

| | | | | |
|---|---|---|---|---|
| | 2. SID | | | |
| | Acid | | 4. SID | |
| | Nucleic | | Aminoacid | 5. SID Amino acid |
| 1. Prey name | SEQ ID | 3. SID Nucleic Acid sequence | SEQ ID | Sequence |

| | | | | |
|---|---|---|---|---|
| Homo sapiens | 27 | TCCCTCAACATCGTCACTGTCACGCTCAACATGGAAAGACATCACTT | 28 | SLNIVTVTLNMERHHFLGISIVGQ |
| dishevelled 1 | | TCTGGGCATCAGCATCGTGGGGCAGAGCACGACCGTGGAGACGGCGG | | SNDRGDGGIYIGSIMKGGAVAADG |
| | | CATCTACATTGGCTCCATCATGAAGGGCGGGGCTGTGGCCGCTGACG | | RIEPGDMLLQVNDVNFENMSNDDA |
| | | GCCGCATCGAGCCCGGCGACATGTTGCTGCAGGTGMTGACGTGACTT | | VRVLREIVSQTGPISLTVAKCWDP |
| | | TGAGAACATGAGCAATGACGATGCCGTGCGGGTGCTGCGGGAGATCG | | TPRSYFTVPRADPVRPIDPA |
| | | TTTCCCAGACGGGGCCCATCAGCCTCACTGTGGCCAAGTGCTGGGAC | | |
| | | CCAACGCCCCGAAGCTACTTCACCGTCCCACGGGCTGACCCGGTGCG | | |
| | | GCCCATCGACCCCGCCGC | | |
| Homo sapiens | 29 | AATATCATCACAGTCACGCTAAACATGGAGAAGTACAACTTCCTGGG | 30 | NIITVTLNMEKYNFLGISIVGQSN |
| dishevelled 2 | | TATCTCCATTGTTGGCCAAAGCMTGAGCGGGGAGACGGAGGCATCTA | | ERGDGGIYIGSIMKGGAVAADGRI |
| | | CATTGGCTCCATCATGAGGGTGGGGCTGTGGCGGCCGACGGGCGCAT | | EPGDMLLQVNDMNFENMSNDDAVR |
| | | TGAGCCAGGGGACATGCTTTTGCAGGTGAATGACATGAACTTTGAGA | | VLRDIVHKPGPIVLTVAKCWDPSP |
| | | ACATGAGCAACGATGACGCTGTGCGGGTGCTGAGGGACATTGTGCAC | | QAYFTL |
| | | AAGCCTGGCCCCATTGTGCTGACTGTGGCCAGTGCTGGGATCCCTCT | | |
| | | CCTCAGGCCTATTTCACTCTCC | | |
| Homo sapiens | 31 | ACGGACTCCACCATGTCACTCAACATCATCACGGTCACTCTCAACAT | 32 | TDSTMSLNIITVTLNMEKYNFLGI |
| dishevelled 3 | | GGAAAAATATAACTTCTTGGGCATCTCCATTGTGGGCCAAAGCAACG | | SIVGQSNERGDGGIYIGSIMKGGA |
| | | AGCGTGGTGACGGCGGCATCTACATTGGCTCTATCATGAAGGGTGGG | | VAADGRIEPGDMLLQVNEINFENM |
| | | GCCGTGGCTGCTGATGGACGCATCGAGCCAGGAGATATGTTGTTACA | | SNDDAVRVLREIVHKPGPITLTVA |
| | | GGTACGAGATCAACTTTGAGAACATGAGTAATGACGATGCAGTCCGG | | KCWDPSPRGCFTLPR |
| | | GTACTGCGGGAGATTGTGCACAAACCGGGGCCCATCACCCTGACTGT | | |
| | | AGCCAAGTGCTGGGACCCMGTCCACGTGGTTGCTTCACATTGCCCAG | | |
| | | G | | |
| prey78847 | 48 | GNTNTTTTTGGAGATACCCCTCCTGGTCTTTTTGGACNTGGCTGGGA | 49 | XXFGDTPPGLFGXGWEPLGPSXXX |
| | | GCCNCTGGGGCCTTCCNNNGNTGNTNTTGANTNNTNCTTCTCTGCAN | | XXXXFSAXSQSXXKXHXGXRWXXG |
| | | GTTCACAATCTTTNTNGIAGNCTCACNNTGGGGNTCGCTGGTANNGC | | *XXTMWXRRFVDTLRVDXRXLSXS |
| | | GGGTGATNATNCACCATGTGGNGGCGTAGATTCGTTGACACTCTTAG | | *GXGXXXXXXPXRXPXXXPSEXAG |
| | | GGTGGACANCAGGNCTTTAAGCANNTCTTGAGGCCNGGGNTANGNAN | | ISGXXTLTGXLEXXXLXXTXXEXX |
| | | AANATNCNGNNCCNAANAGGGANCCCNCNNNANCNCCNTCCGAANGT | | XXXXPXASXXQVRPXLXFSVXRXR |
| | | GCNGGGATCTCAGGATGNNCCACTCTCACNGGANCATTGGAAAGNAN | | KQEAWXX |
| | | TNGNTTGANCCNGACCNGGGNNGAANCCTNTNTNCNCNNAAANCCTT | | |
| | | NTGCATCCTGNNAACAGGTGAGACCNGNCCTGTTNTTCTCTGTTNCC | | |
| | | AGGGNGAGAAAACAACAGGCTTGGANANAAG | | |
| prey78271 | 50 | GACCTCCTAGGCGACCCAGACAATTATACCCTAGCCAACCCCTTAAA | 51 | TS*ATQTIIP*PTP*TPLPTSSPN |
| | | CACCCCTCCCCACATCAAGCCCGAATGATATTTCCTATTCGCCTACA | | DISYSPTQFSDPSLTN*EASLPYY |
| | | CAATTCTCCGATCCGTCCCTAACAAACTAGGAGGCGTCCTTGCCCTA | | YPSSS*Q*SPSSIYPNNKA*YFAH |
| | | TTACTATCCATCCTCATCCTAGCAATAATCCCCATCCTCCATATATC | | *ANHFIDS*PQTSSF*PESEDNQ* |
| | | CAAACAACAAAGCATAATATTTCGCCCACTAAGCCAATCACTTTATT | | ATLLPSLDK*HPYYTSQQS*S*YQ |
| | | GACTCCTAGCCGCAGACCTCCTCATTCTAACCTGAATCGGACCACAA | | LSP*LKTKYSNGPVLVV*TNTPVL |
| | | CCAGTAAGCTACCCTTTTACCATCATTGGACAAGTAGCATCCGTACT | | *TGDENLFPRTNQRKSL*LIAIH* |
| | | ATACTTCACAACAATCCTAATCCTAATACCAACTATCTCCCTAATTG | | HPKLRF*FKLFSVLSWGSRFGYHP |
| | | AAAACAAAATACTCAAATGGGCCTGTCCTTGTAGTATAAACTAATAC | | SIDSPINNRYVFRTLLPATMNIVR |
| | | ACCAGTCTTGTAAACCGGAGATGACCTTTTTCCAAGGACAAATCAGA | | YHKYLTTCST*KPNPHQNPLPMLT |
| | | GAAAAAGTCTTTAACTCCACCATTACCACCCAAAGCTAAGATTCTAA | | SKYSNQPSTITHQLQLQSHPSPTP |
| | | TTTAAACTATTCTCTGTTCTTTCATGGGGAAGCAGATTTGGGTACCA | | AIPTNLPTLNST*YIKPFTVHSTL |
| | | CCCAAGTATTGACTCACCCATCAACAACCGCTATGTATTTCGTACAT | | QSNPFSSPWMTPLR*GSLDHHPP* |
| | | TACTGCCAGCCACCATGAATATTGTACGGTACCATAAATACTTGACC | | NQYPAQECYSPRSGPITLGGS*SE |
| | | ACCTGTAGTACATAAAAACCCAATCCACATCAAAACCCCCTCCCCAT | | LYPTSGSYFRAIKPK*PTRSP*IR |
| | | GCTTACAAGCAAGTACAGCAATCAACCTTCAACTATCACACATCAAC | | HHD |
| | | TGCAACTCCAAAGCCACCCCTCACCCACTAGGATACCAACAAACCTA | | |
| | | CCCACCCTTAACAGTACATAGTACATAAAGCCATTTACCGTACATAG | | |
| | | CACATTACAGTCAAATCCCTTCTCGTCCCCATGGATGACCCCCCTCA | | |
| | | GATAGGGGTCCCTTGACCACCATCCTCCGTGAAATCAATATCCCGCA | | |
| | | CAAGAGTGCTACTCTCCTCGCTCCGGGCCCATAACACTTGGGGGTAG | | |
| | | CTAAAGTGAACTCTATCCGACATCTGGTTCCTACTTCAGGGCCATAA | | |
| | | AGCCTAAATAGCCCACACGTTCCCCTTAAATAAGACATCACGATG | | |
| prey78859 | 52 | TTGCTGCAAAGAGGCCAGAAAGAAGAAACAGGTCAAAGCCTTTGGAT | 53 | LLQRGQKEETGQSLWIL*PRNHRV |
| | | TTTATGACCTAGAAATCACCGGGTGACTTTTTTACCTTAAGAGAAGA | | TFLP*EKTAVKMLTHLWP*H*KQH |
| | | CTGCTGTGAAAATGTTGACCCATCTCTGGCCTTAACATTAAAAGCAG | | ALFTRHLYLNF*VSKYLSMFLPSL |
| | | CATTTTACCAGGCACTTGTACTTGAATTTTTAAGTATCCAAGTACTT | | FCQSFLQSHDKRDKNSQAYSNMKR |
| | | ATCCATGTTTCTCCCCAGCCTCTTCTGTCAGAGCTTTCTCCAGAGCC | | FIFIQAVHIQENKLHS*ILMARRR |
| | | ATGACAAGAGGGATAAGAACTCCCAAGCTTATTCCAACATGAAACGT | | KSDINSXLMEMKSCLHGKVTYGK* |
| | | TTCATTTTCATCCAAGCAGTGCACATCCAAGAGAACAAGCTCCATTC | | P |
| | | CTGAATTTTAATGGCCAGGAGAAGGAACAGTGATATTAACTCANGTC | | |
| | | TGATGGAAATGAAAAGTTGTTTGCATGGGAAGGTTACTTATCGAAAA | | |
| | | TAGCCTCT | | |
| prey1469 | 54 | CGATGAGATTATGACTTCACTCAAGTCTGTTAATGGACAAATAGAAA | 55 | DEIMTSLKSVNGQIESLISPDGSR |
| | | GCCTCATTAGTCCTGATGGTTCTCGTAAAAACCCCGCTAGAAACTCC | | KNPARNCRDLKFCHPELKSGEYWV |
| | | AGAGACCTGAAATTCTGCCATCCTGAACTCAAGAGTGGAGAATACTG | | DPNQGCKLDAIKVFCNMETGETCI |
| | | GGTTGACCCTAACCAAGGATGCAAATTGGATGCTATCAAGGTATTCT | | SANPLNVPRKHWWTDSSAEKKHVW |
| | | GTAATATGGAAACTGGGGAAACATGCATAAGTGCCAATCCTTTGAAT | | FGESMDGGFQFSYGNPELPEDVLD |
| | | GTTCCACGGAAACACTGGTGGACAGATTCTAGTGCTGAGAAGAAACA | | VQLAFLRLLSSRASQNITYHCKNS |
| | | CGTTTGGTTTGGAGAGTCCATGGATGGTGGTTTTCAGTTTAGCTACG | | IAYMDQASGNVKKALKLMGSNEGE |
| | | GCAATCCTGAACTTCCTGAAGATGTCCTTGATGTGCAGCTGGCATTC | | FKAEGNSKFTYTVLEDGCTKHTGE |
| | | CTTCGACTTCTCTCCAGCCGAGCTTCCCAGAACATCACATATCACTG | | WSKTVFEYRTRKAVRLPIVDIAPY |
| | | CAAAAATAGCATTGCATACATGGATCAGGCCAGTGGAAATGTAAAGA | | DIGGPDQEFGVDVGPVCFL* |
| | | AGGCCCTGAAGCTGATGGGGTCAAATGAAGGTGAATTCAAGGCTGAA | | |
| | | GGAAATAGCAAATTCACCTACACAGTTCTGGAGGATGGTTGCACGAA | | |
| | | ACACACTGGGGAATGGACCAAAACAGTCTTTGAATATCGAACACGCA | | |
| | | AGGCTGTGAGACTACCTATTGTAGATATTGCACCCTATGACATTGGT | | |
| | | GGTCCTGATCAAGAATTTGGTGTGGACGTTGGCCCTGTTTGCTTTTT | | |
| | | ATAA | | |
| prey68776 | 56 | ATGGTTTGTCGCGGCTTTGCCTGCTCCAAGAATGCGCTTTGCGCTCT | 57 | MVCGGFACSKNALCALNVVYMLVS |
| | | CAACGTGGTCTACATGCTGGTGAGCTTGTTGCTCATTGGAGTGGCTG | | LLLICVAAWGKGLGLVSSIHIIGG |
| | | CTTGGGGCAAGGGCCTGGGTCTGGTGTCCAGCATCCACATCATCGGC | | VIAVGVFLLLIAVAGLVGAVNHHQ |
| | | GGAGTCATTGCTGTGGGAGTCTTCCTTCTCCTTATTGCAGTGGCTGG | | VLLFFYMIILGLVFIFQFVISCSC |
| | | ACTGGTGGGTGCTGTCAACCACCACCAAGTCCTGCTGTTCTTTTACA | | LAINRSKQTDVINASWWVMSNKTR |
| | | TGATCATCCTTGGTTTGGTCTTCATCTTCCAATTTGTAATCTCTTGC | | DEALERSFDCCGLFNLTTLYQQDY |
| | | TCATGTCTGGCTATTAACCGAAGCAAACAGACAGATGTCATCAATGC | | DFCTAICKSQSPTCQMCGEKFLKH |
| | | TTCTTGGTGGGTCATGAGCAACAAGACTCGGGATGAACTGGAAAGAA | | SDEALKILGGVGLFFSFTEILGVW |
| | | GTTTTGATTGTTGTGGCTTATTCAACCTCACAACCCTGTATCAACAA | | LAMRFRNQKDPRANPSAFL* |
| | | GATTATGATTTCTGCACTGCAATCTGCAAGAGCCAGAGCCCCACATG | | |
| | | CCAGATGTGTGGAGAAAAGTTTCTTAAGCATTCAGACGAAGCCCTGA | | |
| | | AAATCCTAGGGGGTGTTGGACTCTTCTTTAGCTTTACAGAGATCCTT | | |
| | | GGTGTTTGGCTAGCAATGAGATTTCGGAATCAGAAGGATCCTAGAGC | | |
| | | CAACCCCAGTGCCTTTCTATGA | | |
| prey78867 | 58 | GGCAATAGGGGTANTATAAGCCTGAAATTTTTGCAGCCCCCNCCCTC | 59 | GNRGXISLKFLQPPPSXPLXLLYK |
| | | CCNNCCCTTANTTCTACTTTACAAAAGAAATTTTNACAATCTAACCA | | RNFXNXQXRXWXXGGISPAAXTHG |
| | | GAACCAGGGNGTGGNTNANGGGTGGTATTTCTCCAGCTGCTCANACA | | LNRXXPSSSWSWRPQIPRSGPLAX |
| | | CATGGGCTCAACCGACANANTCCNTCTTCCTCCTGGAGCTGGAGGCC | | *QGPXVTSPXQIST*VPLAPLLGP |
| | | CCAAATTCCCAGATCTGGCCCCCTGGCAGNCTCACAGGGACCTTGNG | | GXSSFLILGLVGSRGAXLXWVGKG |
| | | TGACTTCTCCAANGCAGATTTCCACCTAAGTGCCCCTTGCGCCTCTG | | GGXGLWIXXXLPXG |
| | | CTGGGGCCTGGNNAAAGCAGTTTTCTAATTCTTGGCTTGGTTGGNTC | | |
| | | TAGGGGAGCTGNCTTGAANTGGGTGGGGAAAGGCGGNGGTGNAGGNC | | |
| | | TTTGGATTNGACNGATNTTGCCTTNTGGC | | |
| prey78869 | 60 | GCTGATCATACAGAGATGATTAGTGTCACACAGTGTTTTAAACACAT | 61 | ADHTEMISVTQCFKQILATLEFTA |
| | | TCTTGCAACCCTGGAGTTCACTGCAGTAGCAACAGAAGTTAGCTATG | | VATEVSYEILTVV*YVLQLILGSY |
| | | AGATTTTAACAGTAGTATAGTATGTACTACAGTTAATATTAGGTAGC | | DLMLHLCICLHLS*LQVVLCLVLR |
| | | TATGATTTAATGCTGCATCTTTGCATTTGTTTACATTTATCTTGACT | | FVCICFDEF*LFIIGLCIFYGSK* |
| | | ACAAGTGGTATTATGTCTGGTCTTAAGGTTTGTGTGCATATGTTTTG | | *NRLIYIYFM*S*HKPNFFLTF*Y |
| | | ATGAATTTTAACTTTTTATAATAGGTTTGTGTATATTTTATGGCAGT | | F*SMCFI |
| | | AAATGATAAAACAGACTAATCTACATATATTTTATGTAGTCATGACA | | |
| | | TAAACCTAACTTTTTCTTAACTTTTTGATATTTCTAGTCTATGTGTT | | |
| | | TCATCT | | |
| prey78871 | 62 | ATGGGGGAAAATGATCCGCCTGCTGTTGAAGCCCCCTTCTCATTCCG | 63 | MGENDPPAVEAPFSFRSLFGLDDL |
| | | ATCGCTTTTTGGCCTTGATGATTTGAAAATAAGTCCTGTTGCACCAG | | KISPVAPDADAVAAQILSLLPLKF |
| | | ATGCAGATCCTGTTGCTGCACAGATCCTGTCACTGCTGCCATTCAAG | | FPIIVIGIIALILALAIGLGIHFD |
| | | TTTTTTCCAATCATCGTCATTGGGATCATTGCATTGATATTAGCACT | | CSGKYRCRSSFKCIELIARCDGVS |
| | | GGCCATTGGTCTGGGCATCCACTTCGACTGCTCAGGGAAGTACAGAT | | DCKDGEDEYRCVRVGGQNAVLQVF |
| | | GTCGCTCATCCTTTAAGTGTATCGAGCTGATAGCTCGATGTGACGGA | | TAASWKTMCSDDWKGHYANVACAQ |
| | | GTCTCGGATTGCAAAGACGGGGAGGACGAGTACCGCTGTGTCCGGGT | | LGFPSYVSSDNLRVSSLEGQFREE |
| | | GGGTGGTCAGAATGCCGTGCTCCAGGTGTTCACAGCTGCTTCGTGGA | | FVSIDHLLPDDKVTALHHSVYVRE |
| | | AGACCATGTGCTCCGATGACTGGAAGGGTCACTACGCAAATGTTGCC | | GCASGHVVTLQCTACGHRRGYSSR |
| | | TGTGCCCAACTGGGTTTCCCAAGCTATGTGAGTTCAGATAACCTCAG | | IVGGNMSLLSQWPWQASLQFQGYH |
| | | AGTGAGCTCGCTGGAGGGGCAGTTCCGGGAGGAGTTTGTGTCCATCG | | LCGGSVITPLWIITAAHCVYDLYL |
| | | ATCACCTCTTGCCAGATGACAAGGTGACTGCATTACACCACTCAGTA | | PKSWTIQVGLVSLLDNPAPSHLVE |
| | | TATGTGAGGGAGGGATGTGCCTCTGGCCACGTGGTTACCTTGCAGTG | | KIVYHSKYKPKRLGNDIALMKLAG |
| | | CACAGCCTGTGGTCATAGAAGGGGCTACAGCTCACGCATCGTGGGTG | | PLTFNGTSGSLCGSAALPLFQEDL |
| | | GAAACATGTCCTTGCTCTCGCAGTGGCCCTGGCAGGCCAGCCTTCAG | | QLLIEAFL* |
| | | TTCCAGGGCTACCACCTGTGCGGGGGCTCTGTCATCACGCCCCTGTG | | |
| | | GATCATCACTGCTGCACACTGTGTTTATGACTTGTACCTCCCCAACT | | |
| | | CATGGACCATCCAGGTGGGTCTAGTTTCCCTGTTGGACAATCCAGCC | | |
| | | CCATCCCACTTGGTGGAGAAGATTGTCTACCACAGCAAGTACAAGCC | | |
| | | AAAGAGGCTGGGCAATGACATCGCCCTTATGAAGCTGGCCGGGCCAC | | |
| | | TCACGTTCAATGGTACATCTGGGTCTCTATGTGGTTCTGCAGCTCTT | | |
| | | CCTTTGTTTCAAGAGGATTTGCAATTGCTCATTGAAGCATTCTTATG | | |
| | | A | | |
| prey78877 | 64 | NNNNNNNNNNNNNNNNNNNNNNNNNNNNNNNNNNNNNNNNNNNNNNN | 65 | XXXXXXXXXXXXXXXXXXXXXXXX |
| | | NNNNNNNNNNNNNNNNNNNNNNNNNNNNNNNNNNNNNNTCAACGGCA | | XXXXXNGIGCXXXXXXXXXXXXXX |
| | | TAGGTTGTGNNNNNNNNNNNNNNNNNNNNNNNNNNNNNNNNNNNNNN | | XXXXXXXXXXXEEXGRKXLCQXVX |
| | | NNNNNNNNNNNNNNNNNNNNNNNNNNNNNNNNNNNGGAAGAANAAGG | | LKXSXAXGTX*QTXXXLXEVSKXX |
| | | AAGGAAAANCCTTTGCCAAANGGTANTNCTCAAANCNTCNNTGCCTT | | REXRKKFFEKXXGEXRPNLMKPXP |
| | | GNGGNACAATNTAACAAACCNTANTNTNTTTANTTGAAGTNTCAANA | | MXQEXQQIPTNFXD*X*XXLVRRG |
| | | NCNGAGAGAANNNCGGAAIAAAATTTTTTGAAAAANTANNGGGGGAA | | XS |
| | | AANCGTCCAAATTTGATGAAACCTCANCCCATGANTCAGGAGNCTCA | | |
| | | ACAGATTCCAACCAATTTTANGGATTAANATTGAANANATTTGGTCA | | |
| | | GGCGNGGGGNCTCTTN | | |
| prey78879 | 66 | TTTTGCAACCCACTCCCATGGTGTGACGGGCGCTGTGTACAAGGCCC | 67 | FCNPLPWCDGRCVQGPGTYSPWHS |
| | | GGGAACGTATTCACCGTGGCATTCTGATCCACGATTACTAGCCATTC | | DPRLLAIPTSWSRVADSNPDYDAL |
| | | CGACTTCATGGACTCGAGTTGCAGACTCCAATCCGGACTACGACGCA | | YEVRLLSRGRFSLYAPL*HVCSPG |
| | | CTTTATGAGGTCCGCTTGCTCTCGCGAGGTCGCTTCTCTTTGTATGC | | RKGHDDLTS*EWRLPIPPSWSKLG |
| | | GCCATTGTACCACGTGTGTAGCCCTGGTCGTAAGGGCCATGATGACT | | KDQFSSERS*QEKTMSKVGINGFG |
| | | TGACGTCGTGAGAATGGAGATTGCCCATCCCCCCATCCTGGTCTAAG | | RIGRLVLGRLLEVKSN |
| | | CTTGGAAAGGATCAATTTTCATCCGAACGTTCCTGACAGGAGAAAAC | | |
| | | CATCAGTAAAGTTGCTATTAACGGTTTTGGTCGTATCGGTCGACTGG | | |
| | | TGTTGGGTCGATTACTTGAAGTCAAAAGCAACAT | | |
| prey78890 | 68 | CCCNCCACCACCACATNGTGCGCAACTCATGTAACCAAATACCACCT | 69 | PXTTTXWATHVTKYHLIPQ*LMEK |
| | | AATACCCCAATAACTTATGGAAAAATAAATAAACTTAATTTAAAAAA | | *INLI*KKXXVELTFSFMG*YRPY |
| | | AAANGTANGTGGAGCTCACTTTCAGTTTTATGGGATAATATAGGCCC | | W*XPCVVAHSTGNLNFLPFKFLLR |
| | | TATTGGTAAATNCCTTGCGTNGTTTGACATAGCACTGGCAATCTAAA | | NRPWXPSLCMXETXLXL*XSXKXL |
| | | CTTTTTACCTTTTAAATTTTTGCTGAGGAACCGTCCTTGGGANCCCA | | PLVESGR**XRKXPGXSRKXV*CL |
| | | CCCTNGGGATGANCGAAACTNTCCTANTCCTTTAACANAGTTANAAA | | XXLKXP |
| | | CNCTTGCCACTTGTTGAATCTGGTAGGTGATAGTNCAGGAAANCTCC | | |
| | | TGGGCNTTCTAGGAAATNTGTTTAATGCTTGCNCANCCTCAAAATNC | | |
| | | CN | | |
| prey78891 | 70 | TTTACAGTATATTTGTTATAAATATAATACATTTTTGAAAAGCTTAT | 71 | FTVYLL*I*YIFEKLIFN*LEIYI |
| | | TTTTAATTAACTTGAGATTTATATTTTAAACAGATCAACTCAATAGT | | LNRSTQ*LTLYNKEYGNFNAKNRI |
| | | TAACGTTATATAATAAAGAATATGGTAATTTTAATGCCAAAAATCGT | | VK*RTLY*DE*GEKSQNSFQNYIQ |
| | | ATTGTGAAATAGCGCACTCTATACTGAGATGAATCAGGGCAAAAAAG | | SIRKNFFFFLQSYV*VIGTTKY*N |
| | | TCAAAACTCCTTTCAAAACTATATTCAAAGCATCAGAAAAAATTTTT | | MKTL*PKFKTDFKELCEARQENLY |
| | | TTTTCTTTTTACAAAGTTATGTATAAGTCATAGGGACCACCAAATAC | | LTLYRTSQ*SWN |
| | | TGAAATATGAAGACTCTATGACCAAAGTTCAAAACTGATTTTAAGGA | | |
| | | ACTCTGTCAAGCAAGACAGGAAAATTTGTATTTAACACTCTATAGAA | | |
| | | CTTCACAGTAAAGCTGCAATTT | | |
| prey78892 | 72 | ATTTTTATTGAATACTTCTGAATGACAGACACTGTGTCTATTCAGCA | 73 | IFIEYF*MTDTVSIQQIQQ*TCXH |
| | | AATACAACAGTCAACTTGTANGCACCTACCTTNATTCTATNAGCAAA | | LPXFYXETDHQINTCSIX*ILFCL |
| | | CTGACCATCAGATAAATACCTGCAGCATAANGTGAATACTGTTCTGC | | IFHTXFIVLVH*LSF*LGRXKMSH |
| | | TTAATTTTTCACACTTNTTTTATAGTTTTAGTCCATTAGCTTTCTTT | | X*SVIM*HLFLVIDEAFIXSXXYC |
| | | CTGACTAGGAAGGNTNAAGATGTCACATTNGTAATCTGTAATTATGT | | FIIKPEAKPFFFFSLNFXXXXXGK |
| | | AACATTTATTTCTGGTAATAGATGAAGCTTTTATCANAAGCNGTGNG | | *XRLN |
| | | TACTGCTTCATTATTAAACCTGAAGCAAAGCCTTTTTTTTTCTTTTC | | |
| | | CTTGAATTTCTNCCANCNNNCANGGGGAAAATAAATNAGGCTCAATC | | |
| | | T | | |
| prey78893 | 74 | NNNNNNNNNNNNNNNNNNNNNNNNNNNNNNNNNNNNNNNNNNNNNNN | 75 | XXXXXXXXXXXXXXXXXXXXXXXX |
| | | NNNNNNNNNNNNNNNNNNNNNNNNNNNNNNNNNNNNNTCAACGGCAT | | XXXXSTA*VVXXXXXXXXXXXXXX |
| | | AGGTTGTGNNNNNNNNNNNNNNNNNNNNNNNNNNNNNNNNNNNNNNN | | XXXXXXXXXXXXXXEGKTFAKXXX |
| | | NNNNNNNNNNNNNNNNNNNNNNNNNNNNNNNNNNNNGAANAANAANG | | SXXHWXXXQXNXPXXV*LKXQXXR |
| | | AAGGAAAAACCTTTGCCAAANGGTANTNCTGANAGCNTCACTGGCNT | | XKDRKXFLXX*XGXXVQX*XXLXP |
| | | GNGCNACAATNTAACANACCNTANTNTGTTTAATTGAAGTNTCAAAA | | *LXXLXX |
| | | NANCAGAGANAAAGACAGAAAAAANTTTTTGAANAANTAANGGGGGN | | |
| | | AAANCGTCCAANTTTGATNAAANCTCANCCCATGACTNAANAANCTN | | |
| | | ANCANAT | | |
| prey78894 | 76 | ACATATTGCTGGGTTTAACACTCAGGTGATGTCACAATGCCCCAAGT | 77 | TYCWV*HSGDVTMPQVGKSQAEES |
| | | GGGCAAATCACAGGCAGAAGAGTCACAAATTATCAGTGCAGGGCCCA | | QITSAGPSNMSQCSLKANLKEESH |
| | | GCAATATGTCACAATGTTCCCTGAAGGCAAACCTAAAAGAAGAATCA | | KLGVGSCTDNL*LFCL*FAHLGHX |
| | | CATAAGCTAGGTGTTGGGTCCTGCACTGATAATTTGTGACTCTTCTG | | DILX |
| | | CCTGTGATTTGCCCACTTGGGGCATTGNGACATATTGNGGGT | | |
| prey78898 | 78 | NTTGAAAGNGACAAGGGAACAATTTATTACAAATATTGATTGCAAGT | 79 | XEXDKGTIYYKKILIASLLRAGTD |
| | | CTGTTACGTGCAGGCACAGATCACATACATGGTTTTGTTTAACCTTA | | HIHGFV*PYNSXXSWVS*SILKMR |
| | | TAACAGCNCTGNGAGTTGGGTATCATGATCAATTTTGAAAATGAGGA | | KLXLTNXDXVLXVSSGXHNKILSQ |
| | | AACTGANGCTNACGAATTNGGATAGNGTGTTGNAAGTCAGTTCAGGC | | NGWLKQWTFIFSXFWRLXFNYQGX |
| | | TGNCATAATAAAATATCACAGAATGGGTGGCTTAAACAATGGACNTT | | GRXSXXXXLSXXXAXAHPXSLVXX |
| | | TATTTTCTCATNGTTCTGGAGGCTGNACTTCAACTATCAAGGTGNTG | | XGFSXGXGGPPGGVLALXAXXXXR |
| | | GGAGGGNNAGTTTNTNGGNANCNCTCTCTNCTNGGGNGGCAAANGCG | | XXXXXWKP |
| | | CACCCNNTCTCCCTGGTGTNTNNTATNGGCTTTTCCNCNGGTNGGGG | | |
| | | GGGACCCCCNGGGGGNGTCCTTGCCTTGNTNGCCNNAAGNNATNGCC | | |
| | | GANTCNCTTNNTNCNATTGGAAACCCCG | | |
| prey5779 | 80 | AGAGAAGGCTCACGATGGTGGCCGCTACTACCGGGCTGATGATGCCA | 81 | EKAHDGGRYYRADDANVVRDRDLE |
| | | ATGTGGTTCGTGACCGTGACCTCGAGGTGGACACCACCCTCAAGAGC | | VDTTLKSLSQQIENIRSPEGSRKN |
| | | CTGAGCCAGCAGATCGAGAACATCCGGAGCCCAGAGGGAAGCCGCAA | | PARTCRDLKMCHSDWKSGEYWIDP |
| | | GAACCCCGCCCGCACCTGCCGTGACCTCAAGATGTGCCACTCTGACT | | NQGCNLDAILKVFCNMETGETCVY |
| | | GGAAGAGTGGAGAGTACTGGATTGACCCCAACCAAGGCTGCAACCTG | | PTQPSVAQKNWYISKNPKDKRHVW |
| | | GATGCCATCAAAGTCTTCTGCAACATGGAGACTGGTGAGACCTGCGT | | FGESMTDGFQFEYGGQGSDPADVA |
| | | GTACCCCACTCAGCCCAGTGTGGCCCAGAAGAACTGGTACATCAGCA | | IQLTFLRLMSTEASQNITYHCKNS |
| | | AGAACCCCAAGGACAAGAGGCATGTCTGGTTCGGCGAGAGCATGACC | | VAYMDQQTGNLKKALLLKGSNEIE |
| | | GATGGATTCCAGTTCGAGTATGGCGGCCAGGGCTCCGACCCTGCCGA | | IRAEGNSRFTYSVTVDGCTSHTGA |
| | | TGTGGCCATCCAGCTGACCTTCCTGCGCCTGATGTCCACCGAGGCCT | | WGKTVIEYKTTKSSRLPIILDVAP |
| | | CCCAGAACATCACCTACCACTGCAAGAACAGCGTGGCCTACATGGAC | | LDVGAPDQEFGFDVGPVCFL* |
| | | CAGCAGACTGGCAACCTCAAGAAGGCCCTGCTCCTCAAGGGCTCCAA | | |
| | | CGAGATCGAGATCCGCGCCGAGGGCAACAGCCGCTTCACCTACAGCG | | |
| | | TCACTGTCGATGGCTGCACGAGTCACACCGGAGCCTGGGGCAAGACA | | |
| | | GTGATTGAATACAAAACCACCAAGTCCTCCCGCCTGCCCATCATCGA | | |
| | | TGTGGCCCCCTTGGACGTTGGTGCCCCAGACCAGGAATTCGGCTTCG | | |
| | | ACGTTGGCCCTGTCTGCTTCCTGTAA | | |
| prey2805 | 82 | TGGAGCTGATAGTATCCTGGATCTTGGATTAGTAGACTCCTCAGAAA | 83 | GADSILDLGLVDSSEKVQQENRER |
| | | AGGTACAGCAGGAAAACCGAAAGCGCCATGGCTCTAGTCGAAGTGTA | | EGSSRSVVDMDLDDTDDGDDNAPL |
| | | GTAGATATGGATTTAGATGATACAGATGATGGTGATGACAATGCCCC | | FYQPGKRGFYTPRPGKNTEARLNC |
| | | TTTGTTTTACCAACCTGGGAAAAGAGGATTTTATACTCCAAGGCCTG | | FRNIGRILGLCLLQNELCPITLNR |
| | | GCAAGAACACAGAAGCAAGGTTGAATTGTTTCAGAAACATTGGCAGG | | HVIKVLLGRKVNWHDFAFFDPVMY |
| | | ATTCTTGGACTATGTCTGTTACAGAATGAACTCTGTCCTATCACATT | | ESLRQLILASQSSDADAVFSANDL |
| | | GAATAGACATGTAATTAAAGTATTGCTTGGTAGAAAAGTCAATTGGC | | AFAIDLCKEEGGGQVELIPNGVNI |
| | | ATGATTTTGCTTTTTTTGATCCTGTAATGTATGAGAGTTTCCGGCAA | | PVTPQNVYEYVRKYAEHRMLVVAE |
| | | CTAATCCTCGCGTCTCAGAGTTCAGATGCTGATGCTGTTTTCTCAGC | | QPLHAMRKGLLDVLPKNSLEDLTA |
| | | AATGGATTTGCCATTTGCAATTGACCTGTGTAAAGAAGAAGCTGGAG | | EDFRLLVNGCGEVNVQMLISFTSF |
| | | GACAGGTTGAACTCATTCCTAATGGTGTAAATATACCAGTCACTCCA | | NDESGENAEKLLQFKRWFWSIVEK |
| | | CAGAATGTATATGAGTATGTGCGGAAATACGCAGAACACAGAATGTT | | MSMTERQDLVYFWTSSPSLPASEE |
| | | GGTAGTTGCAGAACAGCCCTTACATGCAATGAGGAAAGGTCTACTAG | | GFQPMPSITIRPPDDQHLPTANTC |
| | | ATGTGCTTCCAAAAAATTCATTAGAAGATTTAACGGCAGAAGATTTT | | ISRLYVPLYSSKQILKQKLLLAIK |
| | | AGGCTTTTGGTAAATGGCTGCGGTGAAGTCAATGTGCAAATGCTGAT | | TKNFGFV* |
| | | CAGTTTTACCTCTTTCAATGATGAATCAGGAGAAAATGCTGAGAAGC | | |
| | | TTCTGCAGTTCAAGCGTTGGTTCTGGTCAATAGTAGAGAAGATGAGC | | |
| | | ATGACAGAACGACAAGATCTTGTTTACTTTTGGACATCAAGCCCATC | | |
| | | ACTGCCAGCCAGTGAAGAAGGATTCCAGCCTATGCCCTCAATCACAA | | |
| | | TAAGACCACCAGATGACCAACATCTTCCTACTGCAAATACTTGCATT | | |
| | | TCTCGACTTTACGTCCCACTCTATTCCTCTAAACAGATTCTCAAACA | | |
| | | GAAATTGTTACTCGCCATTAAGACCAAGAATTTTGGTTTTGTGTAG | | |
| prey78905 | 84 | GTTTCTGGGGTGGCTCTAGACCCTAAGAATAACCTGGTGATTTTCCA | 85 | VSGVALDPKNNLVIFHRCDHVWDG |
| | | CAGAGGTGACCATGTCTGGGATGGAAACTCGTTTGACAGCAAGTTTG | | NSFDSKFVYQQIGLGPIEEDTIII |
| | | TTTACCAGCAAATAGGACTCGGACCAATTGAAGAAGACACTATTCTT | | LVIDPNNAAVLQSSEKNLFYLPHG |
| | | GTCATAGATCCAAATAATGCTGCAGTACTCCAGTCCACTGAAAAAAA | | LSIDKDGNYWVTDVALHQVFKLDP |
| | | TCTGTTTTACTTGCCACATGGCTTGAGTATAGATAAAGATGGGAATT | | NNKEGPVLILGRSMQPGSDQNHFC |
| | | ATTGCGTCACAGACGTGGCTCTCCATCAGGTGTTCAAACTGGATCCA | | QPTDVAVDPGTCAIYVSDCYCNSR |
| | | AACAATAAAGAAGGCCCTGTATTAATCCTGGGAAGGAGCATGCAACC | | IVQFSPSGKFITQWGEESSGSSPL |
| | | AGGCAGTGACCAGAATCACTTCTGTCAACCCACTGATGTGGCTCTGG | | PGQFTVPHSLALVPLLGQLCVADR |
| | | ATCCAGGCACTGCAGCCATTTATGTATCAGATGGTTACTGCAACAGC | | ENGRIQCFKTDTKEFVREIKHSSF |
| | | AGGATTGTGCAGTTTTCACCAAGTGCAAAGTTCATCACACAGTGGGG | | GRNVFAISYIPGLLFAVNGKPHFC |
| | | AGAAGAGTCTTCAGGGAGCAGTCCTCTGCCAGGCCAGTTCACTGTTC | | DQEPVQGFVMNFSNCEIIDIFKPV |
| | | CTCACAGCTTCGCTCTTGTGCCTCTTTTGGGCCAATTATGTGTGGCA | | RKHFDMPHDIVASEDGTVYIGDAH |
| | | GACCGGGAAAATGGTCGGATCCAGTGTTTTAAAACTGACACCAAAGA | | TNTVWKFTLTEKLEHRSVKKAGIE |
| | | ATTTGTGAGAGAGATTAAGCATTCATCATTTGGAAGAAATGTATTTG | | VQEIKEAEAVVETKMENKPTSSEL |
| | | CAATTTCATATATACCACGCTTGCTCTTTGCACTGAATGCCAAGCCT | | QKMQEKQKLIKEPCSGVPVVLITT |
| | | CATTTTGGGGACCAAGAACCTGTACAAGCATTTCTGATGAACTTTTC | | LLVIPVVVLLAIAIFIRWKKSRAF |
| | | CAATCGGCAAATTATAGACATCTTCAAGCCACTGCGCAAGCACTTTG | | GADSEHKLETSSGRVLCRFRGKGS |
| | | ATATGCCTCATGATATTCTTGCATCTGAAGATGGGACCGTGTACATT | | GGLNLGNFFASRKGYSRKGFDRLS |
| | | GGAGATGCTCATACCAACACCGTGTGGAAGTTCACCTTGACTGAGAA | | TEGSDQEKEDDGSESEEEYSAPLP |
| | | ATTGGAACATCGATCAGTTAAAAAGGCTGGCATTGAGGTCCAGGAAA | | ALAPSSS* |
| | | TCAAAGAAGCCGAGGCAGTTGTTGAAACCAAAATGGAGAACAAACCC | | |
| | | ACCTCCTCAGAATTGCAGAAGATGCAAGAGAAACAGAAGCTGATCAA | | |
| | | AGAGCCAGGCTCGGGAGTGCCTGTTGTTCTCATTACAACCCTTCTGG | | |
| | | TTATTCCGGTGGTTGTCCTGCTGGCCATTGCCATATTTATTCGGTGG | | |
| | | AAAAAATCAAGGGCCTTTGGAGCAGATTCTGAACACAAACTCGAGAC | | |
| | | GAGTTCAGGAAGAGTACTGGGAAGATTTAGAGGAAAGGGAAGTGGAG | | |
| | | GCTTAAACCTTGGTAATTTCTTTGCAAGCCGTAAGGGCTACAGTCGA | | |
| | | AAAGGCTTTGACCGGCTTAGCACTGAGGGCAGTGACCAAGAGAAAGA | | |
| | | CGATGATGGAAGTGAATCAGAAGAGGAGTATTCAGCACCTCTGCCTG | | |
| | | CGCTCGCACCTTCCTCCTCCTGA | | |
| prey32308 | 86 | GGTGGCTGAGTTCCACACGCACTTGGAGCGGCTGCCTGCCAAGGACA | 87 | VAEFHTELERLPAKDIQTNVYIKH |
| | | TACAGACCAATGTCTACATCAAGCACCCAGTGTCCCTGGAGCAATAC | | PVSLEQYLMEGSYNKVFLAKGNIP |
| | | CTGATGGAGGGCAGCTACAACAAAGTGTTCCTGGCCAAGGGTAACAT | | AESYTFFIDILLDTIRDEIAGCIE |
| | | CCCCGCCGAGAGCTACACCTTCTTCATTGACATCCTGCTCGACACTA | | KAYEKILFTEATRILFFNTPKKMT |
| | | TCAGGGATGAGATCGCTGGGTGCATCGAGAAGGCCTACGAGAAAATC | | DYAKKRGWVLGPNNYYSFASQQQK |
| | | CTTTTCACTGAGGCCACCCGGATCCTCTTCTTCAACACACCCAAAGA | | PEDTTIPSTELAKQVIEYARQLEM |
| | | TGACAGACTACGCCAAGAAGCGAGGGTGGGTCCTGGGCCCCAACAAC | | IV* |
| | | TACTACAGTTTTGCCAGCCAGCAGCAGAAGCCGGAAGACACCACCAT | | |
| | | TCCCTCCACAGAACTGGCCAAACAGGTCATCGAGTATGCCCGGCAGC | | |
| | | TGGAGATGATCGTCTGA | | |
| prey78921 | 88 | TTTTTGAGACGCGGTCTCANNNNNNNNNNNNNNNNNNNNtAINNNNN | 89 | FLRRGLXXXXXXXXXXXXXXXXXR |
| | | NNNNNNNNNNNNNNNNNNNNNNTACGGGAAAAAAAAAANNNAAAAAA | | EKKKKXXKKXXXPFWAXXNLFX*X |
| | | AANGNNCCCNCCCGTTTTGGGCATTNTNAAACCTTTTTNTTTAANNN | | XXXXFFFF*XXFXPFFXXXGXXGX |
| | | NTTNNNANNNANTTTTTTTTTTTTTAANNAAANTTTTNNCCNTTTTT | | XLKTXLXFXFFXFFXXSLFFFFXP |
| | | TTTNGNANTTGGNNNNNGGGGAAANTNATTAAAAACCNTCCTTTNCT | | PFXGXPGAP |
| | | TTTNNTTTTTTNTTTTTTTTTTNNTTTCCCTTTTTTTTTTTTTTNTN | | |
| | | CCTCCCTTTTNGGGGGNCCCGGGGGCCCCTT | | |
| prey78922 | 90 | AAGAACGAGGGAGGTACATGGTCAGTGGAGAAGCGCTGGATGGTGGA | 91 | KNEGGTWSVEKRWMVELSAQPTXX |
| | | CCTGAGTGCGCAGCCCACCNTTCNNCNNTTCTACAAGAACGAGGGAT | | XFYKNEGCTWXXXKXXQXPXXXCX |
| | | GTACNTGGTNANTGGNGAAGNTGNTCCAGNTGCCCTNTTNANANTGT | | GXXCAXFSXXXXLXXXFSWXXALX |
| | | GNCGGNNTNTNTTGTGCCCNCTTTTCCNTGTNTNTTNTNCTNNTTTN | | XFTTFXXLVXXCFXLFXXY*XPFL |
| | | TGNTTTCTCCTGGANGANNGCTTTGNTTTNNTTCACCACCTTTNTTN | | XFFFFFXXFXFFLXXFFXFFXXXX |
| | | NNTTGGTNTNCTGNTGTTTTNTTTTATTTNTNANCTATTGANTCCCT | | XXPXXFFCLXXXXFXXLFXXFFX |
| | | TTCTTGNTTTTTTTTTTTTTTTTTTNNNNATTTTTNTTTTTTTTAGN | | |
| | | CCNTTTCTTTTTNTTTTTCNGTNNTANTCNTNTCTNTCCTNNTNTNT | | |
| | | TTTTTTGTTTGNGNATNATNNTTTTTNNTCNCCTTTTTCNTTNTTTT | | |
| | | TTTTTNGT | | |
| prey68432 | 92 | GGTGCCCACAATCATGGAGGAGGATGCCTTCCAGCTGCCCGGCCTTG | 93 | VPTIMEEDAFQLPGLGTPPITKLT |
| | | GCACGCCACCCATCACCAAGCTCACAGTGCTGGAGGAGGATGAAAAG | | VLEEDEKKPVPWESHNSSETCGLP |
| | | AAGCCGGTGCCCTGGGAGTCCCATAACAGCTCAGAGACCTGTGGCCT | | TLVQTYVLQGDPRAVSTQPQSQSG |
| | | CCCCACTCTGGTCCAGACCTATGTGCTCCAGGGGGACCCAAGAGCAG | | TSDQVLYGQLLGSPTSPGPGHYLR |
| | | TTTCCACCCAGCCCCAATCCCAGTCTGGCACCAGCGATCAGGTCCTT | | CDSTQPLLAGLTPSPKSYENLWFQ |
| | | TATGGGCAGCTGCTGGGCAGCCCCACAAGCCCAGGGCCAGGGCACTA | | ASPLGTLVTPAPSQEDDCVFGPLL |
| | | TCTCCGCTGTGACTCCACTCAGCCCCTCTTGGCGGGCCTCACCCCCA | | NFPLLQGIRVHGMEALGSF* |
| | | GCCCCAAGTCCTATGAGAACCTCTGGTTCCAGGCCAGCCCCTTGGGG | | |
| | | ACCCTGGTAACCCCAGCCCCAAGCCAGGAGGACGACTGTGTCTTTGG | | |
| | | GCCACTGCTCAACTTCCCCCTCCTGCAGGGGATCCGGGTCCATGGGA | | |
| | | TGGAGGCGCTGGGGAGCTTCTAG | | |
| prey78925 | 94 | GNTGATCNTACNGAGATGATTATNNGTCACNNANGGCTNTAAACANC | 95 | XDXTEMIXXHXXL*TXXLQPWR*L |
| | | ANTCTTGCAGCCGTGGAGGTAACTGCAGCAGNNCNANNTGTTNNNTA | | QQXXXXXMRF*XWYSVXYX*XXXA |
| | | TGAGATTTTAACANTGGTATAGTGTGTNCTACNGGTAATNTNNGGNN | | MIXXXXFAFXYIXLDYRXCYVWX* |
| | | GCTATGATTNAANGCTGNATNTTTGCATTTGNTTACATTTNTCTTGA | | GXSAXVLINLXXPXSXXGXGXGRX |
| | | CTACAGGNGGTCTTATGTCTGGNCTTAAGGNTNGAGTGCANATGTNT | | XXLLAI |
| | | TGATTAATCTNANCTNTCCNTNNTCCNTGTNTGGTNCAGGCCANGCC | | |
| | | AGGNTGNCGNNNCTACTGGCTATTTT | | |
| prey78928 | 96 | NNNNNNNNNNNNNNNNNNNNNNNNNNNNNNNNNNNNNNNNNNNNNNN | 97 | XXXXXXXXXXXXXXXXXXXXXXXX |
| | | NNNNNNNNNNNNNNNNNNNNNNNNNNNNNNNNNNNNNNTCAACGGCA | | XXXXXNGIGCXXXXXXXXXXXXXX |
| | | TAGGTTGTGNNNNNNNNNNNNNNNNNNNNNNNNNNNNNNNNNNNNNN | | XXXXXXXXXXXEXEGRKNLCQRVX |
| | | NNNNNNNNNNNNNNNNNNNNNNNNNNNNNNNNNNNNGGAANAAGAAG | | LKXSXAXGTXXQTXXCXXEVSKXX |
| | | GAAGGAAAAACCTTTGCCAAAGGGTANTNCTCAAANCNTCNNTGGCN | | RXXRQKSFRKXXGGKXSXFXXXSX |
| | | TGNGGNACAATNTANCAAACCNTANTNTGTTNANTTGAAGTNTCAAA | | XXSXTSPXSNQF*GXKLXX |
| | | ANACCNGAGANAANGACGGCAAAAGTCTTTTCGAAAAANTANNGGGG | | |
| | | GNAAANCNTCCAANTTTNATNAAANCTCANCCCNTGANTCNNNAACN | | |
| | | TCACCANATTCCAACCAATTTTAGGGANTAAAATTGAANANG | | |
| prey78935 | 98 | NNNNNNNNNNNNNNNNNNNNNNNNNNNNNNNNNNNNNNNNNNNNNNN | 99 | XXXXXXXXXXXXXXXXXXXXXXXX |
| | | NNNNNNNNNNNNNNNNNNNNNNNNNNNNNNNNNNNNNNTCAACGGCA | | XXXXXNGIGCXXXXXXXXXXXXXX |
| | | TAGGTTGTGNNNNNNNNNNNNNNNNNNNNNNNNNNNNNNNNNNNNNN | | XXXXXXXXXXXEXEGRKNLXQXVX |
| | | NNNNNNNNNNNNNNNNNNNNNNNNNNNNNNNNNNNGGAANAAGAAGG | | LKXSXAXGTXXQTXXCXIEXSXDX |
| | | AAGGAAAAACCTTTGNCAAANGGTANTNCTCAAANCNTCANTGGCNT | | REXTEKXF*XNXGGKXSKFDENSX |
| | | GNGGNACAATNTANCAAACCNTANTNTGTTNAATTGAAGNCTCAAAN | | XDSXXSPDSNQFFXIXIEXIWSGX |
| | | GACCNGAGAGAANNGACGGAAAAANTTTTTTGAANAAATANNGGGGG | | G |
| | | GAANCGTCCAAATTTGATGAAAACTCANCCCNTGACTCANGANGNTC | | |
| | | ACCNGATTCCAACCAATTTTTTNGGATTANAATTGAANAAATTTGGT | | |
| | | CAGGCGNGGGGG | | |
| prey67563 | 100 | TCCCGTCCCCCTACCTCCAGTCACACACCTTGACCTCACCCCCAGCC | 101 | PVPLPPVTHLDLTPSPDVPLTIMK |
| | | CTGATGTGCCTCTCACCATCATGAAAAGGAAACTGATGAACACCAAT | | RKLMNTNDLEESRQLTEEIQRHLD |
| | | GATCTGGAGGAGTCCAGGCAGCTCACGGAGGAGATCCAGCGGCATCT | | ARHLIEKSVRKIVSLLAASEAEVE |
| | | GGATGCCAGGCACCTCATTGAGAAGTCAGTGCGTAAGATCGTCTCCT | | QLLSEARAPLTGHSCYPEALLHFR |
| | | TGCTGGCAGCGTCCGAGGCTGAGGTGGAGCAGCTCCTGTCCGAGAGA | | THCFNWHSPTYAYALRHLYVLVNL |
| | | GCCCCGCTCACGGGGCACAGCTGCTACCCAGAGGCCCTGCTGCACTT | | CEKPYPLHRIKLSMDHVCLGHY* |
| | | CCGGACCCACTGCTTCAACTGGCACTCCCCCACGTACGAGTATGCGT | | |
| | | TGAGACATTTGTACGTGCTGGTCAACCTTTGTGAGAGCCGTATCCAC | | |
| | | TTCACAGGATAAAATTGTCCATGGACCACGTGTGCCTTGGTCACTAC | | |
| | | TGA | | |
| prey104794 | 102 | ACGCAAGAACCTAAGCATTTGCTAGAGGGTACAATTATTGCTTCCAA | 103 | TQEPKHLLEGTIIASKIVLTFLGY |
| | | AATAGTCCTTACATTTTTAGGCTATCTTTGAGAGGCTAANTCTTGCC | | L*EAXSCLSFXXLSYXWLEAASTE |
| | | TCTCATTCANTTNACTTTCTTACTNCTGGCTTGAGAATTCTACTGAA | | X*DSWPISPTXXPKMKSIMSFCNR |
| | | GNATGAGACAGCTGGCCAATAAGCCCAACTTGNTNGCCAAAGATGAA | | W*A*VPIETY*AQWQCYNLIFPCX |
| | | ATCCATTATGTCCTTCTGCAACAGATGGTGAGCGTAAGTCCCAATAG | | GS*KKDMLNWINSKVYL*XAKNYS |
| | | AGACATACTGAGCTCAATGGCAGTGCTACAACTTGATTTTCCCTTGC | | CSENPFLF |
| | | CNGGGGAGCTAGAAAAAGGACATGCTCAATTGGATCAATAGTAAAGT | | |
| | | TTACTTATAGNGAGCCAAGAATTACTCCTGTAGTGAGAACCCATTTC | | |
| | | TATTTTC | | |
| prey104797 | 104 | GGGCNTGCTACCTTACNTTATGGCGGAANAAANGTGCAACTGACAAC | 105 | GXATLXYGGXXVQLTTTXRFFKLX |
| | | GACTNATAGATTTTTTAAGTTANAAGNNGTTNGGATCTTTTGGATTT | | XVXIFWIFFFXGXXXXKXXINXXP |
| | | TTTTTTTTTNGGGGNNTTNANCNNCAAAACNNNCCCTAAATNACANG | | PXGWGXFFFFXRXNXXXXGGGFXP |
| | | CCCCCCCNCGGTTGGGGNNNTTTTTTTTTTTTTTTNCGGCNAAACCA | | XPXXXDXGGPPPQXXPPXXXPGGG |
| | | NANANNNNCCGGGGGGGGTTTTTNNCCNNCCCCNCNCTTNCNGGATT | | |
| | | NNGGNGGTCCCCCCCCCCAANGNNNCCCCCCNTANTNCNCCCCNGGG | | |
| | | GGGGGGNG | | |
| prey104799 | 106 | GTGAAGCGCCCTAGTTCCCAAAGGGCGCTAATGAATGCTGTTTCGTC | 107 | VKRPSSQRALMNAVSSSCSPHPIT |
| | | CTCCTGCTCCCCACACCCCATAACTAATAGACTTCAGTTTTTCGGGT | | NRLQFFGFF*TEFRSVARQRXXXX |
| | | TTTTTTAGACGGAGTTCCGCTCTGTCGCCAGGCAGAGGTNNNNNNNN | | XXXXXXXXXXXXXXXXXXXXXXXX |
| | | NNNNNNNNNNNNNNNNNNNNNNNNNNNNNNNNNNNNNNNNNNNNNNN | | XXXXXXXXXXNFNLLYF*IXXXXX |
| | | NNNNNNNNNNNNNNNNNNNNNNNNNNNNNNNNNNNNNNNNNNNNNNN | | XXXXXXXXXXXXXXXXXXXXXXXX |
| | | NNNNNNNNNNCAATTTTAATTTACTTTATTTTTAAATTNNNNNNNNN | | XXXXXXXRCEP |
| | | NNNNNNNNNNNNNNNNNNNNNNNNNNNNNNNNNNNNNNNNNNNNNNN | | |
| | | NNNNNNNNNNNNNNNNNNNNNNNNNNNNNNNNNNNNNNNNNNNNNNN | | |
| | | NNNGCAGGTGTGAGCCA | | |
| prey92121 | 108 | A | 109 | |
| prey104802 | 110 | GTGAAGCGCCCTAGTTNCCAAAGGGCGCTAATGAATGCTGTTTCGTC | 111 | VKKPSXQRALMNAVSSSCSPHPIT |
| | | CTCCTGCTCCCCACACCCCATAACTAATAGACTTNAGTTTTTNGGGT | | NRLXFXGFF*TEFRSVXXXXXXXX |
| | | TTTTTTAGACGGAGTTCCGNTCTGTCGNCNGNNNNNNNNNNNNNNNN | | XXXXXXXXXXXXXXXXXXXXXXXX |
| | | NNNNNNNNNNNNNNNNNNNNNNNNNNNNNNNNNNNNNNNNNNNNNNN | | XXXXXXXXXXNFNLLYF*IXXXXX |
| | | NNNNNNNNNNNNNNNNNNNNNNNNNNNNNNNNNNNNNNNNNNNNNNN | | XXXXXXXXXXXDGLSXPXDPLXLX |
| | | NNNNNNNNNNCAATTTTAATTTACTTTATTTTTAAATTNNNNNNNNN | | LPKLWDXP |
| | | NNNNNNNNNNNNNNNNNNNNNNNNNNNNNNNNNNNNNTNGATCGTCT | | |
| | | CTCTINACCTTGNGATCCACTCCNCCTCNCCCTCCCAAAGTTGTGGG | | |
| | | ATTNCCCAN | | |
| prey78388 | 112 | A | 113 | |
| prey32308 | 114 | CACGGAGTTCGAGCGGCTGCCTGCCAAGGACATACAGACCAATGTCT | 115 | TELERLPAKDIQTNVYIKHPVSLE |
| | | ACATCAAGCACCCAGTCTCCCTGGACCAATACCTGATGGAGGGCAGC | | QYLMEGSYNKVFLAKGNIPAESYT |
| | | TACAACAAAGTGTTCCTGGCCAAGGGTAACATCCCCGCCGAGAGCTA | | FFIDILLDTIRDEIAGCIEKAYEK |
| | | CACCTTCTTCATTGACATCCTGCTCGACACTATCAGGGATGAGATCG | | ILFTEATRILFFNTPKKMTDYAKK |
| | | CTGGGTGCATCGAGAAGGCCTACGAGAAAATCCTTTTCACTGAGGCC | | RGWVLGPNNYYSFASQQQKPEDTT |
| | | ACCCGGATCCTCTTCTTCAACACACCCAAAAAGATGACAGACTACGC | | IPSTELAKQVIEYARQLEMIV* |
| | | CAAGAAGCGAGGGTGGGTCCTGGGCCCCAACAACTACTACAGTTTTG | | |
| | | CCAGCCAGCAGCAGAAGCCGGAAGACACCACCATTCCCTCCACAGAA | | |
| | | CTGGCCAAACAGGTCATCGAGTATGCCCGGCAGCTGGAGATGATCGT | | |
| | | CTGA | | |
| prey104808 | 116 | G | 117 | |
| prey104810 | 118 | TTGTTTTATTGTAAATGTNATAAAATCCAGAACAGCAGAAACATATT | 119 | LFYCKCXQNPEQQKHINQFEILEI |
| | | AATCAGTTTGAAATTTTAGAIATCCTTTAGCACTTGAAPAGAGTATT | | L*HLKKSITNASISHRKSANTN*T |
| | | ACTATGCATCTATATCACATAGAAAGTCAGCGAATACAAACTAGACA | | SRT*FFSGIPG**EYLSIKRSISV |
| | | AGCAGGACATAGTTCTTTTCTGGCATTCCAGGATAATAAGAATATTT | | FLK*LQT*VNTHSFRIGSDWRKKR |
| | | ATCAATTAAAAGGTCAATATCTGTCTTCCTGAAATAACTCCAAACCT | | NTAFCFLFXLSNHREG*TKGKKXE |
| | | GAGTCAACACACATTCTTTTCGGATTGGTTCTGACTGGCGTAAGAAG | | KKVRCSL |
| | | AGAAATACAGCATTTTGTTTTTTATTTTGNTTATCTAACCACACGGA | | |
| | | AGGATAAACAAAGCGCAAAAAGTGNGAGAAAAAGGTTAGATGTTCCT | | |
| | | TGAT | | |
| prey104811 | 120 | CTACTGTGTGCCTTTCTCTAGCTAGCTCATTTTATTCTTGTTATGCA | 121 | LLCAFL*LAHFILVMHFPDNVC*G |
| | | TTTCCCAGACAATGTATGCTGAGGGCCTACTATGCATGAGACACTGT | | PTMHETLS*EQRWFPGGPTTSLSL |
| | | CCTAGGAACAGAGGTGGTTCCCTGGTGGCCCTACTACGTCATTATCA | | WILDCRILN*HIF*WHMS*GRDCS |
| | | TTGTGGATTTTGGATTGCACAATATTGAACTGACACATTTTTTGATG | | RAGESLPPVQTLMFATMFVSG*QL |
| | | GCACATGTCTTGAGGGAGAGACTGCAGTAGGGCAGGTGAATCTTTGC | | SVRLSGTSREKLCYVALDFENEMA |
| | | CACCTGTGCAGACCCTAATGTTTGCCACAATGTTTGTATCTGGTTGA | | LPHPHPPFE |
| | | CAGCTGAGCGTGAGATTCTCCGGGACATCAAGGGAGAAACTGTGTTA | | |
| | | TGTAGCTCTGGACTTTGAAAATGAGATGGCACTGCCCCATCCTCATC | | |
| | | CTCCCTTTGAG | | |
| prey104813 | 122 | TGTAACCTTGGATNACNNANTAACGAGCCNGCCCCCCCCCCANAANT | 123 | CNLGXXXNEPAPPPXXYXXSSXYT |
| | | ATATGANNNCTCCTCCNTTTATACAATTNCTATCAAI4CTGTNTAAA | | IXIXLXKXIXXXPLNNXXTGALXP |
| | | NGGATATNCNNTNTACCCTTGAATAACNCCANAACNGGGGCGCTGNC | | VQPKXXLXLLTXXXLTN*XPTIX* |
| | | CCCTGTACAGCCAAAAATNNNCCTGTANCTTTTGACTNCTNTANTAC | | KPYX*HXRXINT*KDYXLPVFMQS |
| | | TTACCAACTAANGGCCTACTATTNACTGAAAGCCTTACNNATAACAT | | *HIHFXXFFQYF*AT*XAXXXFFP |
| | | ANNCGNNTGATTAATACATAAAAAGACTATGNTCTACCTGTTTTTAT | | KXX |
| | | GCAATCNTGACATATTCATTTTTNTTANTTTTTTCAATATTTCTAGG | | |
| | | CNACATAGGNNGCAAGNTNNNTATTCTTTCCAAAANANTNTAT | | |
| prey70344 | 124 | TGCAAACCCAAAGACTAAAGATTATGGAATATTATGAGAAGAAAGAG | 125 | QTQRLKIMEYYEKKEKQIEQQKKI |
| | | AAACAGATTGAGCAGCAGAAGAAAATTCAGATGTCCAATTTGATGAA | | QMSNLMNQARLKVLRARDDLITDL |
| | | TCAAGCGAGACTCAAAGTCCTCAGAGCAAGAGATGACCTTATCACAG | | LNEAKQRLSKVVKDTTRYQVLLDG |
| | | ACCTACTAAATGAAGCAAAACAGAGACTCAGCAAGGTGGTAAAAGAT | | LVLQGLYQLLEPRMIVRCRKQDFP |
| | | ACAACCAGGTACCAAGTGCTGCTGGATGGACTGGTTCTCCAGGGTTT | | LVKAAVQKAIPMYKIATKNDVDVQ |
| | | GTACCAGTTGCTGGAGCCCCGAATGATTGTTCGTTGCAGGAAACAAG | | IDQESYLPEDIAGGVEIYNGDRKI |
| | | ATTTCCCTCTGGTAAAGGCTGCAGTGCAGAAGGCAATTCCTATGTAC | | KVSNTLESR |
| | | AAAATTGCCACCAAAAACGATGTTGATGTCCAAATTGACCAGGAGTC | | |
| | | CTACCTGCCTGAAGACATAGCTGGTGGAGTTGAGATCTATAATGGAG | | |
| | | ATCGTAAAATAAAGGTTTCCAACACCCTGGAAAGCCGGC | | |
| prey104818 | 126 | G | 127 | |
| prey102423 | 128 | GGGAAATGGAAAGGGACATGGACAGGGATGTGGATCGGATTTCAAGA | 129 | EMERDMDRDVDRISRPNDMYDRSL |
| | | CCTATGGATATGTATGATAGAAGTTTGGATAATGAGTGGGACACAGA | | DNEWIDRDYGRPLDEQESQFRERD |
| | | TTATGGGAGACCACTGGATGAIACAAGAATCACAGTTTCGTGAACGG | | IPSLPPLPPLPPLPPLDRYRDDRW |
| | | GATATTCCATCTCTTCCACCTTTACCGCCCCTCCCACCTCTTCCACC | | REERNREHGYDRDFRDRGELRIRE |
| | | TTTGGATAGATATCGGGATGATAGATGGAGAGAAGAAAGAAATCGAG | | YPERGDTWREKRDYVPDRMDWERE |
| | | AGCATGGGTATGATCGAGATTTCCGTGATAGGGGTGAGTTGAGGATT | | RLSDRWYPSDVDRHSPMAEHMPSS |
| | | CGAGAGTATCCAGAAAGAGGAGATACATGGCGGGAAAAGCGAGATTA | | HHSSEMMGSDASLDSDQGLGGVMV |
| | | TGTTCCTGACAGAATGGACTGGGAAAGAGAACGGTTGTCAGACAGAT | | LSQRQHEIILKAAQELKMLREQKE |
| | | GGTACCCATCTGATGTGGATAGACATTCCCCCATGGCGCAACATATG | | QLQKMKDFGSEPQMADHLPPQEAS |
| | | CCCTCCTCACATCATTCCTCAGAAATGATGGGGTCCGATGCAAGCTT | | RLQNTSSRPGMYPPPGSYRPPPPM |
| | | AGACTCTGACCAAGGCCTTGGAGGGGTAATGGTTCTCAGTCAGAGGC | | GKPPGSIVRPSAPPARSSVPVTRP |
| | | AGCATGAAATCATTTTGAAAGCTGCACAAGAACTGAAAATGCTTCGG | | PVPIPPPPPPPPLPPPPPVIKPQT |
| | | GAACAGAAAGAACAGCTTCAAAAGATGAAAGACTTCGGGTCTGAGCC | | SAVEQERWDEDSFYGLWDTNDEQG |
| | | ACAGATGGCTGACCATCTACCACCTCAGGAATCAAGATTGCAGAATA | | LNSEFKSETAAIPSAPVL |
| | | CATCTTCAAGACCTGGAATGTATCCGCCTCCAGGGTCGTATAGACCT | | |
| | | CCCCCTCCTATGGGCAAACCACCAGGTTCAATTGTAAGACCCTCTGC | | |
| | | TCCACCAGCAAGATCATCTGTTCCTGTGACCAGGCCACCTGTCCCAA | | |
| | | TACCACCACCTCCACCTCCTCCACCTCTACCTCCTCCTCCTCCAGTG | | |
| | | ATAAAGCCACAAACTTCAGCTGTAGAACAGGAACGATGGGATGAAGA | | |
| | | TTCTTTCTATGGGCTCTGGGATACAAATGATGAACAAGGACTGAATT | | |
| | | CAGAATTTAAGTCAGAAACTGCAGCAATTCCATCTGC | | |
| | | TCCAGTATTAC | | |
| prey104821 | 130 | CTTGGTTGGTTTTGATTTTTTTTTTTTAAGCCTTAAAAAACAACCTA | 131 | LGWF*FFFFKP*KTT*XEXLXLKE |
| | | GTNTGAATNGTTATNCTTGAAGGAAAAATAAAACANTTNCTNTTTGA | | K*NXXXLKKSXVSHG*TKXXILQX |
| | | AAAAATCAANNGTTTCNCATGGATAAACAAAANCAAANATTTTGCAA | | XSXKXXXPNXEX*XHXLRAXXXFX |
| | | NTTTNTTCCTTNAAAANCAANGNCCCAAATNGNGAAANCTAAAANCA | | X*XXEG*SPLQXFXXGLXXTPCXX |
| | | TAANTTAAGGGCCANGNGNTTNTTTTTNNTTTAANGCNTCGAAGGAT | | GXC*TPGXPPWGV*RSXXYXXGXX |
| | | AATCNCCTTTGCAANACCCNCNCNANGGGCTCNTTTANACTCCNTGT | | DFPPRPPP* |
| | | NTGCANGGTNTTTGTTGAACNCCCGGANCTCCTCCNTGGGGGGTCTG | | |
| | | AAGGTCNTTNTNTTACNGNNGGGGTTNANAGGACTTTCCACCTCGGC | | |
| | | CACCTCCNTGAGG | | |
| prey78495 | 132 | C | 135 | |
| prey104823 | 134 | G | 137 | |
| prey102110 | 136 | A | 139 | |
| prey104828 | 138 | ATANAGCTGAATTTGTGTCCTTGTGTGACTTTTTGTGAAAAAGNTTA | 141 | IXLNLCPCVTFCEKXYTHCF*NQA |
| | | TACACATGGATTCTAGAATCAGGCTTTATAGTTTATAGAATTTGTTG | | L*FIEFVXXIGLFHSI*TSXRXRC |
| | | NGNGGATAGCGTTATTCCACAGTATTTAGACTTCNNAACGCANTCGT | | IFXTLPTFFXT*XTITPFXXHXL* |
| | | TGCATTTTTAANACATTACCTACTTTTTTTATNACTTAATNCACAAT | | LXXXWFPGITSSQISLFXGLMYXL |
| | | TACCCCCTTCCNGGNGCATAANCTGTAGCTNCNAAANTNCTGCTTTC | | XPPFXNSXKFLKTXTPKMPXGMTP |
| | | CTGCAATTACCTCAAGCCAAATTTCGCTNTTTNAAGGATTAATGTAT | | LQSXEK |
| | | GANCTCANGCCCCCTTTTNNAAATTCATNCAAATTTTTGAAAACCNA | | |
| | | TACCCCAAAGATGCCCGNGGGAATGACCCCCCTNCAAAGTGNTGAAA | | |
| | | AG | | |
| prey104829 | 140 | TGCCCACTCTTCTATGCTGAAGTTACCAGGCAGAGCAGTTTTCTTAC | 143 | CPLFYAEVTRQSSFLTSQLLCLVY |
| | | AAGTCAGCTACTCTGCTTGGTTTATTTTAGGTTTTGGTACTTCACGT | | FRFWYFT*ALLEGTSVLISLVLRR |
| | | AAGCACTGTTAGAAGGTACAAGTGTATTAATATCACTAGTTTTGAGG | | LGTFVFNIFRMCSKLFSHFFFFLA |
| | | CGCTTGGGTACATTTGTTTTTAATATATTTAGAATGTGCAGTAAACT | | NLLF*VQLLSXQSTXRMX*HIYWX |
| | | TTTTTCTCATTTTTTTTTCTTTTTAGCAAACTTGTTATTTTAGGTCC | | NTINDE*XI*XLLYSGGGHXXXGX |
| | | AATTATTGAGTTGNCAGTCTACTGNGAGAATGANATGACATATCTAC | | XLXPXXRGPX |
| | | TGGGANAATACCATAAATGATGAATAGTTNATTTGANAACTTTTATA | | |
| | | CTCAGGGGGGGGNCATTGNNTTTNNGGAGNGANNCTTCNCCCCNCNN | | |
| | | NCCGGGGACCNNNTT | | |
| prey100907 | 142 | ATGTGTTTTTATGGATCTAAGTTAAATCTTTTGGCAATATATAAAAA | 145 | MCFYGSKLNLLAIYKNVNSKLYLL |
| | | TGTAAATAGTAAACTTTATTTATTAAGAATGTCATCTTTTTTAATTT | | RMSSFLIYIYTIVHLILYFFYTVL |
| | | ATATTTACACAATTGTTCATCTAATTTATTTTTTCTATACAGTTTTA | | NTQTYFAVHDIFILFSWICFPILF |
| | | AATACTCAGACATATTTTGCTGTTCATGATATTTTTATCCTGTTCTC | | SLISITGWISQIIMSETEIFC |
| | | ATGGATTTGTTTTCCCATACTGTTTTCTCTGATCTCAATTACAGGTT | | |
| | | GGATCTCACAAATAATAATGTCAGAGACAGAAATATTTTGC | | |
| prey104833 | 144 | CTAGTTAAGGAAATGAGCTCTGCTAGCTGTGCCATAGAAATAGGGAA | 147 | LVKEMSSASCAIEIGKNPKATIYT |
| | | AAACCCAAAAGCTACAATTTATACAGATTCAAAATATGCCCTCTCAG | | DSKYALSGLHAHVAIWKEQGFLPA |
| | | GACTCCATGCTCATGTGGCCATCTGGAAGGAACAGGGATTTTTACCA | | KDTPKECGPQILALLAAVHLSQEI |
| | | GCAAAAGATACCCCTAAGGAATCTGGACCACAGATCCTGGCCCTGCT | | AVVHGQGHQRTREKMPSKTGGWIK |
| | | CTAAGCTGTACACCTATCACAGGAAATAGCTGTAGTCCATGGCCAGG | | QPGPLPCRELLMALNPSLPNTLPT |
| | | GACACCAAAGGACCAGGGAGAAAATGCCCAGCAAAACAGGAGGGTGG | | HKKPKKEKEWAI |
| | | ATCAAACAGCCAGGACCACTACCCTGTAGGGAACTCCTCATGGCCCT | | |
| | | TAACCCATCTTTACCCAACACCTTACCCACCCACAAAAAACCCAAGA | | |
| | | AGGAAAAGGAATGGGCTATTN | | |
| prey13439 | 146 | GCTTTTAGGCATATTCTAATTAATGAAAGTAAACAATCTGCTGCTCA | 149 | AFRHILINESKQSAAQQGAFLR |
| | | ACAGGGGGCTTTCCTCAGAGAATATCTTTATGTTTACAAGAATGTAA | | EYLYVYKNVSQLSPDGPLPQLP |
| | | GTCAGCTGTCACCAGATGGTCCTTTGCCACAGCTTCCTTTACCGTAT | | LPYINSSATRVFFGHDRRPADG |
| | | ATTAACAGTTCAGCAACACGGGTTTTTTTTGGCCATGACAGACGACC | | EKQAATHVSLDQEYIDSESSQQW |
| | | AGCGGATGGTGAAAAACAAGCAGCTACTCATGTAAGTCTTGATCAAG | | RELEEQVVSVVNKGVIPSNFHP |
| | | AATATGATTCTGAATCCTCTCAGCAGTGGCGAGAACTTGAGGAACAA | | TQYCLNSYSDNSRFPLAVVEEP |
| | | GTTGTTTCTGTGGTTAACAAAGGAGTAATTCCATCCAATTTTCATCC | | ITVEVAFRNPLKVLLLLTDLSL |
| | | CACACAATACTGTTTGAACAGTTACTCAGATAATTCAAGATTTCCAC | | LWKFHPKDFSGKDNEEVKQLVT |
| | | TTGCAGTTGTAGAAGAACCAATTACAGTGGAAGTGGCTTTTAGAAAC | | SEPEAMIGAEVISEFLINGEESK |
| | | CCTTTGAAAGTTCTACTTTTGTTGACTGATTTGTCATTGCTTTGGAA | | VARLKLFPHHIGELHILGVVYN |
| | | GTTTCATCCTAAAGATTTCAGTGGAAACGATAATGAAGAAGTTAAAC | | LGTIQGSMTVDGICALPGCHTG |
| | | AACTAGTTACAACTGAACCTCAAATGATTGGACCTGAAGTTATTTCA | | KYSLSMSVRGKQDLEIQGPRLN |
| | | GAGTTCTTAATTAATGGCCAACAATCAAAAGTGGCAAGACTAAAGCT | | NTKEEKTSVKYGPIDRRLDPIIT |
| | | CTTTCCCCATCACATAGGGGAGCTGCATATTCTGGGAGTTGTTTATA | | EEMPLLEVFFIHFPTGLLCGEI |
| | | ATCTTGGCACTATTCAGGGCTCTATGACAGTAGATGGCATTGGTGCT | | RKAYVEFVNVSKCPLTGLKVVS |
| | | CTTCCCGGATGTCACACAGGAAAATATTCCTTCAGTATGTCAGTCCG | | KRPEFFTFGGNTAVLTPLSPSA |
| | | AGGGAAGCAGGATTTAGAAATTCAAGGTCCTCGACTTAACAACACAA | | SENCSAYKTVVTDATSVCTALI |
| | | AAGAAGAGAAAACATCTGTTAAATATGGCCCTGATCGACGTTTAGAT | | SSASSVDFGIGTGSQPEVIPVP |
| | | CCCATAATCACAGAAGAAATGCCACTGTTGGAGGTGTTCTTTATACA | | LPDTVLLPGASVQLPMWLRGPD |
| | | TTTTCCTACAGGGCTTCTCTGTGGAGAAATCCGAAAAGCATATCTAG | | EEGVHEINFLFYYESVKKQPKI |
| | | AATTTGTCAATGTCAGCAAATGTCCACTTACTGGATTGAAGGTTGTT | | RHRILRHTAIICTSRSLNVRAT |
| | | TCTAAACGTCCAGAGTTCTTTACTTTCGGTGGTAATACTGCTGTTCT | | VCRSNSLENEEGRGGNMLVFVD |
| | | AACACCACTAAGTCCCTCAGCTTCTGAGAATTGTAGTGCTTACAAGA | | VENTNTSEAGVKEFHIVQVSSS |
| | | CTGTTGTGACAGATGCTACCTCTGTGTGTACAGCACTCATATCATCA | | SKHWKLQKSVNLSENKDAKLAS |
| | | GCTTCTTCTGTAGACTTTGGCATTGGCACAGGAAGTCAACCAGAGGT | | REKGKFCFKAIRCEKEEA |
| | | GATTCCTGTTCCCCTTCCTGACACTGTTCTTCTACCCGGAGCCTCAG | | |
| | | TCCAGCTGCCAATGTGGTTACGTGGGCCTGATGAAGAAGGTGTCCAT | | |
| | | GAAATTAACTTTTTGTTTTACTATGAAAGTGTCAGCAGCCAAAAATA | | |
| | | CGGCACAGAATATTAAGACACACTGCAATTATTTGTACCAGTCGGTC | | |
| | | TTTAAATGTACGGGCCACTGTCTGCAGAAGTAATTCTCTTGAAAATG | | |
| | | AAGAAGCCAGAGGAGGCAATATGCTAGTCTTTGTGGATGTGGAAAAT | | |
| | | ACCAATACTAGTGAAGCACGCGTTAAGGAATTCCACATAGTGCAAGT | | |
| | | ATCAAGTAGTAGCAAACACTGGAAGTTACAGAAATCTGTAAATCTTT | | |
| | | CTGAAAACAAAGATGCCAAACTTGCCAGTAGGGAGAAGGCAAAGTTT | | |
| | | TGCTTTAAGGCAATAAGATGTGAGAAAGAAGAAGCGGC | | |
| prey104840 | 148 | A | 151 | |
| prey104843 | 150 | G | 153 | |
| prey104844 | 152 | N | 155 | |
| prey104845 | 154 | G | 157 | |
| prey68432 | 156 | CCTCCTGCTGTTGCTCACCTGCCTCTGTGGAACTGCCTGGCTCTGTT | 159 | LLLLLTCLCGTAWLCCSPNRKN |
| | | GCAGCCCCAACAGGAAGAATCCCCTCTGGCCAAGTGTCCCAGACCCA | | PLWPSVPDPAHSSLGSWVPTLM |
| | | GCTCACAGCAGCCTGGGCTCCTGGGTGCCCACAATCATGGAGGAGGA | | EEDAFQLPGLGTPPITKLTVLE |
| | | TGCCTTCCAGCTGCCCGGCCTTGGCACGCCACCCATCACCAAGCTCA | | EDEKKPVPWESHNSSETCGLPT |
| | | CAGTGCTGGAGGAGGATGAAAAGAAGCCGGTGCCCTGGGAGTCCCAT | | LVQTYVLQGDPRAVSTQPQSQS |
| | | AACAGCTCAGAGACCTGTGGCCTCCCCACTCTGGTCCAGACCTATGT | | GTSDQVLYGQLLGSPTSPGPGH |
| | | GCTCCAGGGGGACCCAAGAGCAGTTTCCACCCAGCCCCAATCCCAGT | | YLRCDSTQPLLAGLTPSPKSYE |
| | | CTGGCACCAGCGATCAGGTCCTTTATGGGCAGCTGCTGGGCAGCCCC | | NLWFQASPLGTLVTPAPSQEDD |
| | | ACAAGCCCAGGGCCAGGGCACTATCTCCGCTGTGACTCCACTCAGCC | | CVFGPLLNFPLLQGIRVHGMEA |
| | | CCTCTTGGCGGGCCTCACCCCCAGCCCCAAGTCCTATGAGAACCTCT | | LGSF* |
| | | GGTTCCAGGCCAGCCCCTTGGGGACCCTGGTAACCCCAGCCCCAAGC | | |
| | | CAGGAGGACGACTGTGTCTTTGGGCCACTGCTCAACTTCCCCCTCCT | | |
| | | GCAGGGGATCCGGGTCCATGGGATGGAGGCGCTGGGGAGCTTCTAG | | |
| prey104850 | 158 | CCACCAGCAAAAAGATTGTTAGCATTTTTTTAGCAGTGATGTATTTA | 161 | PPAKRLLAFF*Q*CIYQXYIMLLY |
| | | TCAATNATACATAATGCTATTGTACACTTCTTAATAGACTATGGTAT | | TS**TMV*CNHNLCTGKPTNS*DS |
| | | AGTGTAACCATAATTTATGCACTGGGAAACCAACAAATTCATGAGAT | | LYCSALELSLPCF*GIPVYLVCSL |
| | | TCGCTTTATTGTAGTGCTCTGGAATTGAGCCTGCCGTGTTTCTGAGG | | YIKPHFIG*ETKA*SLSNFPAXGX |
| | | TATACCTGTGTATTTAGTATGTTCCTTGTATATAAAACCCCATTTTA | | XHPNKXXGGGXPXRPPLXXGGAPX |
| | | TAGGGTAGGAAACTAAGGCTTAGAGTTTGAGCAATTTTCCCGCATNA | | XKXXEXDVXKX*XGGG |
| | | GGNANANACCACCCNAATAAANNCCNGGGGGGGGGCNTGCCAAANAG | | |
| | | ACCCCCCCTNNNANGGGGGGGGGCTCCTNTNNTAAAAANAANNGAGN | | |
| | | NTGACGTTCANAAANNNTAAANGGGTGGGGGA | | |
| prey104854 | 160 | TGGTATGCTGTTGAAGGAGTAGTGAGAGGGGAAATCCTTGCCTATTT | 163 | WYAVEKGVVRGEILAYF*S*LE |
| | | CTGATCTTAACTGGAAAGATTCTAGTTTCTTGACATTAAGTATGATG | | RF*FLDIKYDVGCGCFVLXMFF |
| | | TTGGCTGTGGGTGTTTTGTTTTGTANATGTTCTTTATCAAGCTGGGG | | IKLGKLQLLLVGCPFVLLTNIS |
| | | AAGTTGCAGCTATTGCTAGTTGGATGCCCCTTTGTATTGCTGACTAA | | LYGFTTICIFIRLLMDIWTVST |
| | | TATTTCATTGTATGGGTTTACCACAATTTGTATATTCATTCGTCTNT | | SWVYK*SHYEHGYASVQVFVWT |
| | | TGATGGACATTTGCATTGTTTCTACTTCTTGGGTGTACAAATAAAGC | | HAFFSDEQRHRNDMAGS |
| | | CACTATGAACATGGGTATGCAAGTGTACAAGTCTTTGTGTGGACACA | | |
| | | TGCCTTCTTTTCTGATGAGCAAAGACACAGGAATGATATGGCTGGAT | | |
| | | CATT | | |
| prey78679 | 162 | GAATGCATCTGTTGAATTATTCCTAGATGCAAATAAACTGCCCACAC | 165 | NASVELFLDANKLPTPVVEQVA |
| | | CTGTAGTAGAACAAGTTGCACAAATAAATGATAATTCAGAACTCGAC | | QINDNSELELTSVVENTFPAQD |
| | | TTGACATCAGTTGTGCAAAATACTTTTCCAGCACAAGATATTGTGCA | | IVHTVTVKRKRGKSQPNCALKE |
| | | CACTGTTACAGTGAAACGGAAACGTGGAAAATCACAGCCAAACTGTG | | HSMSNIASVKSPYEAENSGEEL |
| | | CTCTGAAAGAACACTCTATGTCTAATATAGCCAGCGTCAAGAGTCCT | | DQRYSKAKPMCNTCGKVFSEAS |
| | | TATGAGGCGGAGAACTCCGGGGAAGAGCTGGATCAGAGGTATTCCAA | | SLRRHMRIHKGVKPYVCHLCGK |
| | | GGCCAAGCCAATGTGTAACACATGTGGGAAAGTGTTTTCAGAAGCCA | | AFTQCNQLKTHVRTHTGEKPYK |
| | | GCAGTTTGAGAAGGCACATGAGAATACATAAAGGAGTCAAACCTTAC | | CELCDKGFAQKCQLVFHSRMHH |
| | | GTCTGCCACTTATGTGGAAAGGCATTTACCCAATGTAACCAGCTGAA | | GEEKPYKCDVCNLQFATSSNLK |
| | | AACGCATGTAAGAACTCATACAGGTGAGAAGCCATACAAATGTGAAT | | IHARKHSGEKPYVCDRCGQRFA |
| | | TGTGTGATAAAGGATTTGCTCAGAAATGTCAGCTAGTCTTCCATAGT | | QASTLTYHVRRHTGEKPYVCDT |
| | | CGCATGCATCATGGTGAAGAAAAACCCTATAAATGTGATGTATGCAA | | CGKAFAVSSSLITHSRKHTGEK |
| | | CTTACAGTTTGCAACTTCTAGCAATCTCAAGATTCATGCAAGGAAGC | | PYICGICGKSFISSGELNKHFR |
| | | ATAGTGGAGAGAAGCCATATGTCTGTGATAGGTGTGGACAGAGATTT | | SHTGERPFICELCGNSYTDIKN |
| | | GCTCAAGCCAGCACACTGACCTATCATGTCCGTAGGCATACTGGAGA | | LKKHKTKVHSGADKTLDSSAED |
| | | AAAGCCTTATGTATGTGATACCTGTGGGAAGGCATTTGCTGTCTCTA | | HTLSEQDSIQKSPLSETMDVKP |
| | | GTTCTCTTATCACTCATTCTCGAAAACATACAGGTGAAAAACCATAC | | SDMTLPLALPLGTEDHHMLLPV |
| | | ATATGTGGTATTTGTCGGAAAAGTTTTATTTCCTCAGGAGAGCTCAA | | TDTQSPTSDTLLRSTVNGYSEP |
| | | CAAACACTTTCGGTCCCATACAGGAGAAAGACCATTTATCTGCGAAT | | QLIFLQQLY* |
| | | TATGTGCAAATTCTTACACAGATATTAAAAATTTAAAGAAGCACAAA | | |
| | | ACAAAAGTCCATTCTGGTGCAGATAAAACTCTAGACTCCAGTGCAGA | | |
| | | GGATCATACTTTGAGTGAACAGGATTCCATACAAAAAAGTCCTTTAT | | |
| | | CAGAAACTATGGATGTGAAGCCTTCTGATATGACTTTACCATTAGCT | | |
| | | CTTCCACTTGGGACTCAGGACCATCACATGCTTCTCCCTGTCACGGA | | |
| | | TACTCAGTCTCCTACATCAGATACATTGTTGAGGTCAACTGTGAATG | | |
| | | GGTATTCAGAACCACAGTTGATTTTTTTACAACAATTATACTGA | | |
| prey78564 | 164 | A | 167 | |
| prey104856 | 166 | GCTGCAGGGGTGNGCNCACANAGGGNACCCANGAAGANGGAGGTATG | 169 | AAGVXXXRXPXKXEVWLLPGGK |
| | | GCTCCTGCCTGGAGGAAAGNNAACACGCCCCCNNGGTCCCCCAGGAA | | XTRPXGPPGREGPHSSXSTXXY |
| | | GGGAGGGTCCCCACTCATCAANCTCTACCNACNTCTATCCTATTNAT | | PIXFLN*FXXXXXTCEXXXXDX |
| | | TTTCTNAATTAATTTTNNCNTTTNTANANGACATGTGAANGCNAATN | | SALXXLXXTXXLVGWAVLLXXX |
| | | ATNTGACTANTCTGCACTNTGNNNACTTATNGNNACCGNTCNACTNG | | XPAXXAKXALA |
| | | TNGGATGGGCTGTACTCCTNGNCCNTCANNCNCCCGCCNGAANAGCC | | |
| | | AAGNTNGCTTTGGCT | | |
| prey78440 | 168 | A | 171 | |
| prey51725 | 170 | TGGGAGTATTGGAGTTGGACAGCCCTGGCAGTTTGGTGGAGGAATAA | 173 | GSIGVGQPWQFGGGINSVSYCNQG |
| | | ACAGTGTCTCTTACTGTAATCAGGGATGTGCGAATTCCTGGCTCGCT | | CANSWLADKFCDQACNVLSCGFDA |
| | | GATAAGTTCTGTGACCAAGCATGCAATGTCTTGTCCTGTGGGTTTGA | | GDCGQIDHFHELYKVILLPNQTHY |
| | | TGCTGGCGACTGTGGGCAAGATCATTTTCATGAATTGTATAAAGTGA | | IIPKGECLPYFSFAEVAKRGVEGA |
| | | TCCTTCTCCCAAACCAGACTCACTATATTATTCCAAAAGGTGAATGC | | YSIDNPTIRHASTANKWKTIHLIM |
| | | CTGCCTTATTTCAGCTTTGCAGAAGTAGCCAAAAGAGGAGTTGAAGG | | HSGMNATTIHFNLTFQNTNDEEFK |
| | | TGCCTATAGTGACAATCCAATAATTCGACATGCTTCTATTGCCAACA | | NQITVEVDTREGPKLNSTAQKGYE |
| | | AGTGGAAAACCATCCACCTCATAATGCACAGTGGAATGAATGCCACC | | NLVSPITLLPEAEILFEDIPKEKR |
| | | ACAATACATTTTAATCTCACGTTTCAAAATACAAACGATGAAGAGTT | | FPKFKRHDVNSTRRAQREVKIPLV |
| | | CAAAATGCAGATAACAGTGGAGGTGGACACAAGGGAGGGACCAAAAC | | NTSLLPKDAQLSLNTLDLQLEHGD |
| | | TGAATTCTACAGCCCAGAAGGGTTACGAAAATTTAGTTAGTCCCATA | | ITLK |
| | | ACACTTCTTCCAGAGGCGGAAATCCTTTTTGAGGATATTCCCAAAGA | | |
| | | AAAACGCTTCCCGAAGTTTAAGACACATGATGTTAACTCAACAAGGA | | |
| | | CAGCCCAGGAACAGGTGAAAATTCCCCTGGTAAATATTTCACTCCTT | | |
| | | CCAAAAGACGCCCAGTTGAGTCTCAATACCTTGGATTTGCAACTGGA | | |
| | | ACATGGAGACATCACTTTGAAAG | | |
| prey104864 | 172 | ACAGNTCCAGCTCTTTNCNTTANAGGGAAAAAGGGTGNNCTTATNCA | 175 | TXPALXXXGKKGXLXHYGXILXVX |
| | | CTACGGACNNATTTTANAAGTTNAATTANGCCTGGATCNNTGGGATN | | LXLDXWDXXPPLXLGVLPAGPLLS |
| | | TNNTGCCNCCGTTGNGCCTAGGAGTTCTACCTGCCGGTCCTCTNCTG | | SPGSXRDXXT*DCLIF*TFFXFFG |
| | | AGCTCTCCNGGATCCNAGAGGGACCANNTTACTTAAGATTGCCTTAT | | XXXXPXXPXGGXPGGXXR*IHXPR |
| | | TTTTTAAACTTTTTTTTNTTTTTTTGGANNGGNAGNCTTNCCCTNAA | | XXGXXXXLXXKNRPQXXKXXXXGA |
| | | NCCCCNGGGGGGGANNGCCCGGGGGNGANCNCCGTTAAATTCACNCC | | PXXRGKXL |
| | | CCCCGGGNGNNGGGGTGNNANNAANNNCTTANANNCAAAAACCGNCC | | |
| | | CCAANGGNNGAAAANNNGGNAGCNCGGGGCCCCNNANNNCCGGGGAA | | |
| | | AANTTTTGG | | |
| prey12054 | 174 | A | 177 | |
| prey102131 | 176 | A | 179 | |
| prey104867 | 178 | CTTTCCTAGTATNTNCTGTGCTGNGTNGGCTTTGAAGGGAACNCCAN | 181 | VC*YXLCXVGFEGNXXCLLXXP |
| | | ATGCCTCCTTCNTGNTCCTCTCNGANTGTGTCCTTGNNCTTTAGCTG | | LXXCPXXLAGISXXSYXXSTMP |
| | | GGATTTCANANANNTCTTATNCTNNATCAACAATGCCTACNTTATGG | | TLWSX*FTXRPXXISXSPALXS |
| | | TCNNACTGATTTACNNGCAGGCCTNCTNNGATATCCNNATCACCTGC | | XXLLLGXTQAIXXTXXSFWXDT |
| | | CCTTNCGTCGGNNGANCTATTACTTGGTNGCACTCAGGCGATTNANT | | ILKXPSPIVPCTXXXXPSXXSX |
| | | NCACCTTNNGGAGCTTTTGGANCGACACNATCCTCAAAANTCCCTCA | | PEGIXXFFLQXXXXI |
| | | CCTATAGTTCCCTGCACTNNCNTTATNAGNCCCTCGGANANATCCTN | | |
| | | TCCGGAGGGCATTNGGNCCTTCTTTCTACAANGACANAANNGGATCC | | |
| | | T | | |
| prey54064 | 180 | A | 183 | |
| prey104869 | 182 | GTGAAGCGCCCTAGTTCCCAAAGGGCGCTAATGAATGCTGTTTCGTC | 185 | VKRPSSQRALMNAVSSSCSPHPIT |
| | | CTCCTGCTCCCCACACCCCATAACTAATANACTTTANTTTTTNGGGT | | NXLXFXGFF*TEFRSXAXXXXXXX |
| | | TTTTTTAGACGGAGTTCCGCTCTGNCGCCAGNNNNNNNNNNNNNNNN | | XXXXXXXXXXXXXXXXXXXXXXXX |
| | | NNNNNNNNNNNNNNNNNNNNNNNNNNNNNNNNNNNNNNNNNNNNNNN | | XXXXXXXXXXNFNLLYFAIXXXXX |
| | | NNNNNNNNNNNNNNNNNNNNNNNNNNNNNNNNNNNNNNNNNNNNNNN | | XXXXXXXXXXXXXXXXXXXXXXXX |
| | | NNNNNNNNNNNCAATTTTAATTTACTTTATTTTTAAATTNNNNAAAN | | XXXXXXXXXXXX |
| | | NNNNNNNNNNNNNNNNNNNNNNNNNNNNNNNNNNNNNNNNNNNNNNN | | |
| | | NNNNNNNNNNNNNNNNNNNNNNNNNNNNNNNNNNNNNNNNNNNNNNN | | |
| | | NNNNNNNNNNNNNNNNNNNNNNNTT | | |
| prey104870 | 184 | GGNGAGCTCATNTTTAAGGAAGATGAAGGGATNTCTANGTTTTTTTT | 187 | GEVXFKEDEGXSXFFFGFD*STVN |
| | | CGGTTTTGATTAAAGTACTGTCAATTTCTGAATACATANAGCTTTAA | | F*IHXALNFSIVHFNALHKILXAH |
| | | ATTTTTCGATAGTGCATTTCAACGCTCTTCATAAAATTTTAAANGCC | | *XLLXXK*DNVNTXPSXGRWGEDR |
| | | CACTGANTACTACTTTGNAGNAAATGAGATAATGTTAACACCTNCCC | | XIKDX*RNRLQLCXKISNQ*XXGD |
| | | NTCTGNTGGGAGGTGGGGCGAGGACCGGANTATTAAGGATNTTTAAA | | EWXPREXYSCSLXXLLLXFTSC*Y |
| | | GGAATCGACTTCAGCTCTGCANAAAGATTAGCAATCAGTAANGATTN | | GNSGX |
| | | GGGGATGAATGGNCACCTAGAGAAGNTTATTCTTGCTCTCTGNTATN | | |
| | | NCTGCTACTGNATTTTACATCTTGTTAATATGGNAATTCAGGTTNTG | | |
| | | C | | |
| prey104949 | 186 | TTCCTGAAGCCACAGGAAAGCACCCTGGAGTCCTTTGTATACCACTA | 189 | FLKPQESTLESFVYHYGNFAGKLS |
| | | TGGAAATTTTTAAGGCAAGCTCTCAAGACTTGAAGAGTTGGAATTCT | | RLEELEFSAFK*CLKFVLKKQYFI |
| | | CGGCATTTAAATGATGCCTGAAGTTTGTTTTGAAAAAGCAATATTTC | | ALFNCEATAQQILLIFLKS*IKGK |
| | | ATTGCTCTTTTCAATTGTGAAGCAACTGCTCAGCAAATATTACTCAT | | YTFKTKIDQNDPSFKGFCLFWKNK |
| | | TTTTTTAAAAAGTTGAATTAAACGGAAATATATTTTTAAAACCAAAA | | TKMLKLLNYREQMYSET*F*TNI* |
| | | TAGATCAAAATGATCCCAGTTTTAAGGGATTTTCCCTATTTTGGAAG | | NKAG*HLRLX |
| | | AATAAAACTAAAATGTTAAAACTGTTGAATTACAGAGAACAGATGTA | | |
| | | CTCTGAGACATAATTTTAAACAAATATTTAAAATAAGGCAGGNTAAC | | |
| | | ATTTGCGTTTANGCCT | | |
| prey1469 | 188 | TATGACTTCACTCAAGTCTGTTAATGGACAAATAGAAAGCCTCATTA | 191 | MTSLKSVNGQIESLISPDGSRK |
| | | GTCCTGATGGTTCTCGTAAAAACCCCGCTAGAAACTGCAGAGACCTG | | NPARNCRDLKFCHPELKSGEYW |
| | | AAATTCTGCCATCCTGAACTCAAGAGTGGAGAATACTGGCTTGACCC | | VDPNQGCKLDAIKVFCNMETGE |
| | | TAIACCAAGGATGCAAATTGGATGCTATCAAGGTATTCTGTAATATG | | TCISANPLNVPRKHWWTDSSAE |
| | | GAAACTGGGGAAACATGCATAAGTGCCAATCCTTTGAATGTTCCACG | | KKHVWFGESMDGGFQFSYGIATPE |
| | | GAAACACTGGTGGACAGATTCTAGTGCTGAGAAGAAACACGTTTGGT | | LPEDVLDVQLAFLRLLSSRASQ |
| | | TTGGAGAGTCCATGGATGGTGGTTTTCAGTTTAGCTACGGCAATCCT | | NITYHCKNSIAYMDQASGNVKK |
| | | GAACTTCCTGAAGATGTCCTTGATGTGCAGCTGGCATTCCTTCGACT | | ALKLMGSNEGEFKAEGNSKFTY |
| | | TCTCTCCAGCCGAGCTTCCCAGAACATCACATATCACTGCAAAAATA | | TVLEDGCTKHTGEWSKTVFEYR |
| | | GCATTGCATACATGGATCAGGCCAGTGGAAATGTAAAGAAGGCCCTG | | TRKAVRLPIVDIAPYDIGGPDQ |
| | | AAGCTGATGGGGTCAAATGAAGGTGAATTCAAGGCTGAAGGAAATAG | | EFGVDVGPVCFL* |
| | | CAAATTCACCTACACAGTTCTGGAGGATGGTTGCACGAAACACACTG | | |
| | | GGGAATGGAGCAAAACAGTCTTTGAATATCGAACACGCAAGGCTGTG | | |
| | | AGACTACCTATTGTAGATATTGCACCCTATGACATTGGTGGTCCTGA | | |
| | | TCAAGAATTTGGTGTGGACGTTGGCCCTGTTTGCTTTTTATAA | | |
| prey50431 | 190 | TGGATGCACCATTGGCTCCTGTTTGAAATGAGCAGGCACTCCTTGGA | 193 | WMHHWLLFEMSRHSLEQKPTDAPP |
| | | GCAAAAGCCCACTGACGCTCCACCGAAAGAGTCACTGGAACTGGAGG | | KESLELEDPSSGLGVTKQDLGPVP |
| | | ACCCGTCTTCTGGGCTGGGTGTGACCAAGCAGGATCTGGGCCCAGTC | | M* |
| | | CCCATGTGA | | |
| prey104876 | 192 | GCATACTCCTGCATGTGCAAGGGGATTTGGGGTTGCTACCTTTATGG | 195 | AYSCMCKGIWGCYLYGFL*LRGRL |
| | | GTTTCTTTAACTGAGGCGTAGATTATTCCTGACAAAAGGTGAAGATT | | FLTKGEDFSXLCYHLFLHXIWVFS |
| | | TCTCANAACTGTGTTACCACCTATTTTTACACCANATATGGGTGTTC | | QLPGAGGCVI*YVNEHXMWS*XKP |
| | | TCACAACTGCCTGGTGCTGGTGGGTGTGTGATTTAGTATGTTAATGA | | R*NPAPYWV*LVLVSFVHTLFFRS |
| | | GCATATNATGTGGTCCTAGTTNAAACCTAGGTAAAATCCCGCTCCCT | | LTXPXPFXXVXTXXXGXFISXRXP |
| | | ATTGGGTCTAGTTGGTCTTAGTCAGCTTTGTCCATACNCTGTTTTTC | | PPPYLPXXK |
| | | AGGAGCCTTACCCNCCCACANCCCTTTANTCANGTCNAAACTNCCTN | | |
| | | CCNGGGANTTTTTATTTCTNCCCGNGNCCCCCCCCCCCCTTATTTGC | | |
| | | CNNNNTTNAAAG | | |
| prey105012 | 194 | GATGGACTGTGCCCTGAGCTATTGCTGATCCCCCCGCCTCTCTCTAA | 197 | DGLCPELLLIPPPLSNRGILGPVQ |
| | | CCGTGGAATCCTGGGGCCTGTCCAGAGCCCCTGTCCTTCCCGGGACC | | SPCPSRDPAPIPTEPGCLLVEATA |
| | | CTGCACCTATACCTACTGAGCCAGGCTGCCTGCTGGTAGAGGCCACA | | TEEGPGNMEIIVETVAGTLTPGAP |
| | | GCAACTGAAGAGGGACCAGGGAACATGGAGATCATTGTGGAGACAGT | | GETPGTNTVASRPVGQAALRFCLG |
| | | AGCTGGAACCCTGACCCCAGGTGCTCCTGGAGAGACCCCAGGTACAA | | YF*FCINMMRTQGSILFLYLEMLS |
| | | ACACAGTTGCAAGCAGGCCAGTGGGACAAGCAGCTCTGAGGTTTTGT | | FGLHLVHVD*R |
| | | CTGGGTTATTTTTGATTCTGTATAAATATGATCAGAACACAAGGTAG | | |
| | | CATCCTATTTTTATACTTGGAGATGTTGTCATTTGGTTTGCACTTGG | | |
| | | TGCATGTTGATTGAAGGCT | | |
| prey102333 | 196 | ATGCCCCTACCTCCCGGCAGCCTCTCCAGGCCCAGAACTTTCTCCAG | 199 | MPLPPGSLSRPRTFSSQPPQTKLM |
| | | TCAGCCTCCACAGACCAAGCTCATGACTCACAATGGCCTATTTACGC | | THNGLFRPIPYLTAVSADEATASQ |
| | | CCATACCCTACCTCACGGCAGTCTCTGCAGATGAGGCTACTGCCTCA | | QPPQAQLHRYNGLFRPSSCLPAFS |
| | | CAACAGCCTCCACAGGCACAGCTCCACCGTTACAATGGCCTCTTTAG | | PGPELSQVDLTRPSSCFLAASPGP |
| | | ACCCAGCTCCTGCCTCCCAGCCTTCTCTCCAGGCCCTGAGCTTTCTC | | APASWQPLQAQPLPPVSLYSPNIC |
| | | AAGTCGACCTCACCAGCCCCAGCTCATGCTTCTTGGCAGCCTCTCCA | | LTADSSGPVSASLWTTQAKLPTFQ |
| | | GGCCCAGCTCCTGCATCTTGGCAGCCTCTCCAGGCCCAGCCTCTGCC | | QLLHTQLLPPSGLFRPSSCLTRAF |
| | | TCCTGTCAGCCTCTACAGTCCCAACATCTGCCTCACAGCAGATTCTT | | PGPTFVSWQPSLATFLPVSQQPRQ |
| | | CAGGCCCAGTATCTGCCTCACTGTGGACCACCCAAGCCAAGCTCCCA | | AQVLPHTGLSTSSSCLTVASPGPA |
| | | ACCTTTCAGCAGCTTCTACACACCCAGCTCCTGCCACCCAGTGGCCT | | PVPGRHLQAQNFLKSASLVPTAAS |
| | | CTTTAGGCCAAGCTCATGCCTCACAAGGGCCTTTCCAGGCCCAACTT | | WWPMKAQNLLKLTSPGPAPASCQR |
| | | TTGTCTCATGGCAACCTTCCCTGGCCACATTCCTGCCTGTCTCCCAG | | LQAQPLPHGGFSRPSSSSGLSLQA |
| | | CAGCCTAGACAGGCCCAGGTCTTGCCTCACACTGGCCTGTCTACATC | | QLLPHNSLFWPSSCPAHSDQCRPK |
| | | CAGCTCTTGCCTCACGGTGGCCTCTCCAGGCCCAGCTCCTGTCCCAG | | TSSSQTLQACLLPGGMNRPSFDLR |
| | | GACGTCATCTCCAGGCCCAAAACTTCCTCAAGTCAGCCTCTCTAGTC | | TASAGPALASQGLLQAQLLPHGGC |
| | | CCAACTGCTGCCTCCTGGTGGCCTATGAAGGCCCAAAATCTCCTCAA | | PRPSFCLPASSLNRHSSSLTVAHL |
| | | GTTGACCTCTCCAGGCCCAGCTCCTGCCTCCTGTCAGCGTCTACAGG | | GPTHDCRAISRPSACLLADS |
| | | CCCAACCTCTGCCTCATGGGGGCTTCTCCAGGCCCAGCTCTTCCTCT | | |
| | | GGGCTGAGTCTACAGGCACAACTGCTGCCTCACAACAGCCTTTTTTG | | |
| | | GCCCAGTTCCTGTCCAGCTCACAGCGACCAATGTAGGCCCAAAACTT | | |
| | | CCTCAAGTCAAACTCTCCAGGCCTGCCTTCTTCCTGGTGGCATGAAC | | |
| | | AGGCCCAGCTTTGACTTGAGAACAGCCTCTGCAGGCCCTGCTCTTGC | | |
| | | CTCCCAGGGGCTTCTCCAGGCCCAGCTCTTGCCTCATGGCGGCTGCC | | |
| | | CCAGGCCAAGTTTCTGCCTGCCTGCCAGCAGCCTCAACAGGCACAGC | | |
| | | TCTTCCCTCACAGTGGCCCATTTAGGCCCAACTCATGACTGTCGGGC | | |
| | | CATTTCCAGGCCTAGTGCCTGCCTCCTGGCTGACTCTTGA | | |
| prey5779 | 198 | CATCCGGAGCCCAGAGGGAAGCCGCAAGAACCCCGCCCGCACCTGCC | 201 | IRSPEGSRKNPARTCRDLKMCHSD |
| | | GTGACCTCAAGATGTGCCACTCTGACTGGAAGAGTGGAGAGTACTGG | | WKSGEYWIDPNQGCNLDAIKVFCN |
| | | ATTGACCCCAACCAAGGCTGCAACCTGGATGCCATCAAAGTCTTCTG | | METGETCVYPTQPSVAQKNWYISK |
| | | CAACATGGAGACTGCTGAGACCTGCGTGTACCCCACTCAGCCCAGTG | | NPKDKRHVWFGESMTDGFQFEYGG |
| | | TGGCCCAGAAGAACTGGTACATCAGCAAGAACCCCAAGGACAAGAGG | | QGSDPADVAIQLTFLRLMSTEASQ |
| | | CATGTCTGGTTCGGCGAGAGCATCACCGATGGATTCCAGTTCGAGTA | | NITYHCKNSVAYMDQQTGNLKKAL |
| | | TGGCGGCCAGGGCTCCGACCCTGCCGATGTGGCCATCCAGCTGACCT | | LLKGSNEIEIRAEGNSRFTYSVTV |
| | | TCCTGCGCCTGATGTCCACCGAGGCCTCCCAGAACATCACCTACCAC | | DGCTSHTGAWGKTVIEYKTTKSSR |
| | | TGCAAGAACAGCGTGGCCTACATGGACCAGCAGACTGGCAACCTCAA | | LPIIDVAPLDVGAPDQEFGFDVGP |
| | | GAAGGCCCTGCTCCTCAAGGGCTCCAACGAGATCGAGATCCGCGCCG | | VCFL* |
| | | AGGGCAACAGCCGCTTCACCTACAGCGTCACTGTCGATGGCTGCACG | | |
| | | AGTCACACCGGAGCCTGGGGCAAGACAGTGATTGAATACAAAACCAC | | |
| | | CAAGTCCTCCCGCCTGCCCATCATCGATGTGGCCCCCTTGGACGTTG | | |
| | | GTGCCCCAGACCAGGAATTCGGCTTCGACGTTGGCCCTGTCTGCTTC | | |
| | | CTGTAA | | |
| prey104882 | 200 | CATATTATTACGCTGTTTTTAGAAGCAGTTTGACAAATAGTGATTGT | 203 | HIITLFLEAV*QIVIVFVFTNGES |
| | | GTTTGTTTTTACAAATGGTGAATCAGTTAGAAAAATAAAACTTCAGT | | VRKIKLQFI*PLSFTLKQYVFQII |
| | | TTATTTAGCCATTATCATTTACATTAAAACAATATGTTTTTCAAATA | | *LASSDTLFHTF*FWFNSKFIIXD |
| | | ATATAATTGGCATCAAGTGATACACTTTTTCATACTTTTTAGTTTTG | | HNALSSFSFAIL*KTWPFYGRGXX |
| | | GTTTAATTCAAAATTTATAATAGNTGACCATAATGCTTTATCTTCTT | | LLKIF*NEXXXXPXXXPXGPFPPX |
| | | TTTCATTTGCTATTTTATGAAAAACNTGGCCGTTTTATGGCCGGGGG | | LEGXP |
| | | NNGANGCTCCTGAAAATTTTTTAAAATGAANTTTNCCNAANNCCNNG | | |
| | | GGNGGNNCCTNGGGGACCTTTTCCNCCTTNCTTGGAAGGNCNNCCCC | | |
| prey04886 | 202 | TTACTCTGCACATTGTAAAGACTTAATGTGGCTCACTGGCAGAGGTT | 205 | LLCTL*RLNVAHWQRFQDMG*GHD |
| | | TCAGGATATGGGATAGGCACATGATCTGGTGGAAACTGTGGTCCCAG | | LVETVVPVPRVXRTSLVQGFPYSX |
| | | TGCCAAGGGTGTANAGAACATCTCTGGTCCAAGGTTTCCCTTACAGC | | PCLF*GPWRVCKLL*PSQHLPFIG |
| | | TNTCCCTGCTTATTCTGAGGTCCCTGGAGGGTTTGCAAGTTGCTATA | | PYNXERP*TLCRSCRELXNTYTSX |
| | | ACCTTCCCAACACTTGCCTTTTATAGGCCCTTATAACANAGAAAGGC | | LXPAFWPLCLIGKNFDCWEDXXVN |
| | | CCTAAACTTTGTGCAGGAGTTGCAGGGAACTATNAAATACATACACC | | *YGNH |
| | | TCTNTCCTCCNCCCTGCCTTTTGGCCTCTTTGCTTAATAGGAAAGAA | | |
| | | CTTCGATTGTTGGGAAGATNCTNGGGTGAATTAGTATGGGAATCAC | | |
| prey93097 | 204 | A | 207 | |
| prey104890 | 206 | TGGTACTCTCACTTGAGCNACTGCCCCGTTNGTNNTNCNTCGCATCN | 209 | WYSHLSXCPVXXXSHXCXTVSXWX |
| | | GTGCTGNACAGTGTCCANNTGGNTNANCNTGTCCTGNTNTGAGGNGG | | XXSXXEXGXXMFAIXXHVCPXXCX |
| | | GACANTANATGTTTGCTATTNNGNGNCATGTATGCCCANCGNACTGC | | XHXLXX*XPXXCLELPXXXRDTHX |
| | | TGNGNTCATGNACTCNTACNATGANCGCCTTGNNTNTGTCTTGAACT | | XLXLFXXLDXXLSMXX*XXLYS*S |
| | | ACCNGNNNTNNTTCGTGACACTCATNGAANTTTGTNTTTATTTNNTN | | XGIXLPWSSKXXVLPAXAIXPPXL |
| | | NCTTGGATTNNNCGCTCAGTATGANGANATGATTNNNCTTGTACTCC | | GLNLCASXSXLX |
| | | TGATCTTTNGGGATTTNNCTACCCTGGTCTTCAAAANNNTNGGTATT | | |
| | | ACCTGCTTNAGCCATTGNCCCCCCGNNACTTGGTCTTAACCTTTGTG | | |
| | | CCTCCNNNTCNNATTTATAN | | |
| prey102198 | 208 | A | 211 | |
| prey8065 | 210 | GTCCAAACTCCCCATTGGGGATGTGGCCACCCAGTACTTTGCTGACA | 213 | SKLPIGDVATQYFADRDMFCAGRV |
| | | GGGACATGTTCTGTGCTGGCCGAGTACCTGAGGAGGATCTGAAGAGG | | PEEDLKRTMMACGGSIQTSVNALS |
| | | ACAATGATGGCCTGTGGAGGCTCAATCCAGACCAGTGTGAATGCTCT | | ADVLGRCQVFEETQIGGERYNFFT |
| | | GTCAGCAGATGTGCTGGGTCGATGCCAGGTGTTTGAAGAGACCCAGA | | GCPKAKTCTFILEGGAEQFMEETE |
| | | TTGGAGGCGAGAGGTACAATTTTTTTACTGGCTGCCCCAAGGCCAAG | | RSLHDAIMIVRRAIKNDSVVAGGG |
| | | ACATGCACCTTCATTCTCCGTGGCGGCGCCGAGCAGTTTATGGAGGA | | AIEMELSKYLRDYSRTIPGKQQLL |
| | | GACAGAGCGGTCCCTGCATGATGCCATCATGATCGTCAGGAGGGCCA | | IGAYAKALEIIPRQLCDNAGFDAT |
| | | TCAAGAATGATTCAGTGGTGGCTGGTGGCGGGGCCATTGAGATGGAA | | NILNKLRARHAQGGTWYGVDINNE |
| | | CTCTCCAAGTACCTGCGGGATTACTCAAGGACTATTCCAGGAAAACA | | DIADNFEAFVWEPAMVRINALTAA |
| | | GCAGCTGTTGATTGGGGCTTATGCCAAGGCCTTGGAGATTATCCCAC | | SEAACLIVSVDETIKNPRSTVDAP |
| | | GCCAGCTGTGTGACAATGCTGGCTTTGATGCCACAAACATTCTCAAC | | TAAGRGRGRGRPH* |
| | | AAGCTGCGGGCTCGGCATGCCCAGGGGGGTACATGGTATGGAGTAGA | | |
| | | CATCAACAACGAGGACATTGCTGACAACTTTGAAGCTTTCGTGTGGG | | |
| | | AGCCAGCTATGGTGCGGATCAATGCGCTGACAGCAGCCTCTGAGGCT | | |
| | | GCGTGCCTGATCGTGTCTGTAGATGAAACCATCAAGAACCCCCGCTC | | |
| | | GACTGTGGATGCTCCCACAGCAGCAGGCCGGGGCCGTGGTCGTGGCC | | |
| | | GCCCCCACTGA | | |
| prey104894 | 212 | GGGGAGTGTCTGTATATCCTTTAAAACTTTGTTTACAAAGTTTACAC | 215 | GECLYIL*NFVYKVYTHTVCLQKQ |
| | | ACATACTGTATGTCTACAGAAACAATAGTTTTCCTTTGCATTTTTAA | | *FSFAFLN*KQYCSAACFSNNI** |
| | | ACTAGAAACAATATTGTTCAGCAGCCTGCTTTTCTAACAATATATAA | | YITFIETAGYFLIVEAEYSPDQLQ |
| | | TAATACATAACATTTATTGAGACAGCTGGTTATTTTCTCATAGTGGA | | WQRGVKLQQYISSYFLLCIHMQAH |
| | | GGCAGAGTATTCCCCTGACCAATTACAATGGCAGAGGGGTGTGAAAC | | FLTEDTMK*T*LVYLVVWFLNVDI |
| | | TACAGCAATATATATCCTCGTATTTTCTCCTCTGTATACATATGCAG | | IKLSFKIK |
| | | GCACATTTCCTGACAGAAGATACTATGAAGTGAACTTAACTAGTGTA | | |
| | | TCTAGTAGTTTGGTTTTTAAATGTTGACATTATCAAATTGTCCTTTA | | |
| | | AAATAAAAAG | | |
| prey104896 | 214 | TGGACTTGGAATCTGGACTGCCTACTGGATTCTCACGACTTACCAAC | 217 | WTWNLDCLLDSHDLPTDIFDWCYP |
| | | CGATATCTTTGACTGGTGCTACCCAAACTGTTTTCATGTCAGGGGAC | | NCFHVRGPRETDTCTLHCXSCCLK |
| | | CTAGAGAGACAGATACTTGTACATTGCATTGCANATCCTGCTGCTTA | | SEVHIAXVTCFKPEPTNWGFDSPE |
| | | AAGTCAGAAGTTCACATTGCANATGTCACTTGCTTCAAGCCAGAACC | | GRADXYLGKIXDHPXXXXXXXXXX |
| | | GACCAACTGGGGCTTTGACAGCCCTGAGGGGCGAGCGGATCANTATC | | XXXXXXXXXXXXXXXXXXXXXXXF |
| | | TTGGCAAAATTATNGACCATCCCNNNNNNNNNNNNNNNNNNNNNNNN | | GR |
| | | NNNNNNNNNNNNNNNNNNNNNNNNNNNNNNNNNNNNNNNNNNNNNNN | | |
| | | NNNNNNNNNNNNNNNNNNNNNNNNNNCCTTTGGAAGGCT | | |
| prey102355 | 216 | ATGACAGCCATCATCAAAGAGATCGTTAGCAGAAACAAAAGGAGATA | 219 | MTAIIKEIVSRNKRRYQEDGFDLD |
| | | TCAAGAGGATGGATTCGACTTAGACTTGACCTATATTTATCCAAACA | | LTYIYPNIIAMGFPAERLEGVYRN |
| | | TTATTGCTATGGGATTTCCTGCAGAAAGACTTGAAGGCGTATACAGG | | NIDDVVRFLDSKHKNHYKIYNLCA |
| | | AACAATATTGATGATGTAGTAAGGTTTTTGGATTCAAAGCATAAAAA | | ERHYDTAKFNCRVAQYPFEDHNPP |
| | | CCATTACAAGATATACAATCTTTGTGCTGAAAGACATTATGACACCG | | QLELIKPFCEDLDQWLSEDDNHVA |
| | | CCAAATTTAATTGCAGAGTTGCACAATATCCTTTTGAAGACCATAAC | | AIHCKAGKGRTGVMICAYLLHRGK |
| | | CCACCACAGCTAGAACTTATCAAACCCTTTTGTGAAGATCTTGACCA | | FLKAQEALDFYGEVRTRDKKGVTI |
| | | ATGGCTAAGTGAAGATGACAATCATGTTGCAGCAATTCACTGTAAAG | | PSQRRYVYYYSYLLKNHLDYRPVA |
| | | CTGGAAAGGGACGAACTGGTGTAATGATATGTGCATATTTATTACAT | | LLFHKMMFETIPMFSGGTCNPQFV |
| | | CGGGGCAAATTTTTAAAGGCACAAGAGGCCCTAGATTTCTATGGGGA | | VCQLKVKIYSSNSGPTRREDKFMY |
| | | AGTAAGGACCAGAGACAAAAAGGGAGTAACTATTCCCAGTCAGAGGC | | FEFPQPLPVCGDI |
| | | GCTATGTGTATTATTATAGCTACCTGTTAAAGAATCATCTGGATTAT | | |
| | | AGACCAGTGGCACTGTTGTTTCACAAGATGATGTTTGAAACTATTCC | | |
| | | AATGTTCAGTGGCGGAACTTGCAATCCTCAGTTTGTGGTCTGCCAGC | | |
| | | TAAAGGTGAAGATATATTCCTCCAATTCAGGACCCACACGACGGGAA | | |
| | | GACAAGTTCATGTACTTTGAGTTCCCTCAGCCGTTACCTGTGTGTGG | | |
| | | TGATATCAA | | |
| prey104899 | 218 | GAAGAGCCTGAGGACTGGATCTTCCTTGGAATATACNGAAAGCACAA | 221 | EEPEDWIFLGIYXKHKRSWAWVES |
| | | AAGGAGCTGGGCCTGGGTGGAAAGTTGACACATTAGTGAAGAGTGTG | | *HISEECVGHCXHLTLXS*LLGVF |
| | | TTGGTCACTGCANTCATCTNACACTGCANTCGTGATTGCTTGGAGTT | | MICLV*F*ASGALESFNI*KCAAQ |
| | | TTTATGATTTGTTTAGTGTAGTTTTGAGCTTCAGGTGCACTCGAAAG | | NKTLRTTRLWSTCSSHXIFLDGAF |
| | | TTTTAATATTTGAAAATGTGCTGCTCAAAATAAAACTTTAAGAACCA | | WSQKHLTEQNRLSXFTDXMDSXLI |
| | | CTCGTTTGTGGAGCACCTGCTCTTCACATAANATTTTTCTAGATGGA | | TAK**H |
| | | GCTTTTTGGTCTCAAAAGCACTTAACAGAGCAGAACCGGTTATCCNG | | |
| | | ATTTACAGACNAAATGGATAGTTNTTTGATCACTGCTAAGTAATAGC | | |
| | | ATAC | | |
| prey104909 | 220 | TGGGGGAAAGGGGAGGAAGGGAGGGAGGAAAGAAGATTTAGACCCAA | 223 | WGKGEEGREERRFRPKLTXIXFSX |
| | | ACTAACCCNCATCANTTTCTCCNGCTNAGATTCANTATNANAAAGGA | | XDSXXXRTXTGSTIIERCCGLWYF |
| | | CTAANACAGGGAGTACAATTATTGAGAGGTGTTGTGGCTTATGGTAT | | LIFLP*TLFIYIFRV*KYFPKSXS |
| | | TTTTTAATTTTCCTACCTTAAACATTGTTTATTTATATTTTTAGGGT | | LLLEELLALY*IFIXAKLLLXTIS |
| | | TTAAAAATACTTCCCAAAAAGCANCTCATTATTATTAGAGGAACTGT | | QIVFCETYFLXNVXISFLYXPPXG |
| | | TAGCCTTATACTAAATATTTATCANTTAAAAATTATTATTAANTACC | | M*GXXXFXKNPX |
| | | ATTTCTCAAATTGTGTTCTGTGAAACATATTTCTTGNGAAATGTACN | | |
| | | TATTTCTTTTTTATACNCCCCCCCNANAGGGATGTGAGGNNCCNATT | | |
| | | NATTTNNGAAAAATCCNNCC | | |
| prey102479 | 222 | A | 225 | |
| prey78905 | 224 | GGAGTATACTTGTTACCAGGCCAGGTTTCTGGGGTGGCTCTAGACCC | 227 | GVYLLPGQVSGVALDPKNNLVIFH |
| | | TAAGAATAACCTGGTGATTTTCCACAGAGGTGACCATGTCTGGGATG | | RGDHVWDGNSFDSKFVYQQIGLGP |
| | | GAAACTCGTTTGACAGCAAGTTTGTTTACCAGCAAATAGGACTCGGA | | IEEDTILVIDPNNAAVLQSSEKNL |
| | | CCAATTGAAGAAGACACTATTCTTGTCATAGATCCAAATAATGCTGC | | FYLPHGLSIDKDGNYWVTDVALHQ |
| | | AGTACTCCAGTCCAGTGAAAAAAATCTGTTTTACTTGCCACATGGCT | | VFKLDPNNKEGPVLILGRSMQPGS |
| | | TGAGTATAGATAAAGATGGGAATTATTGGGTCACAGACGTGGCTCTC | | DQNHFCQPTDVAVDPGTGAIYVSD |
| | | CATCAGGTGTTCAAACTGGATCCAAACAATAAAGAAGGCCCTGTATT | | GYCNSRIVQFSPSGKFITQWGEES |
| | | AATCCTGGGAAGGAGCATGCAACCAGGCAGTGACCAGAATCACTTCT | | SGSSPLPGQFTVPHSLALVPLLGQ |
| | | GTCAACCCACTGATGTGGCTGTGGATCCAGGCACTGGAGCCATTTAT | | LCVADRENGRTQCFKTDTKEFV |
| | | GTATCAGATGGTTACTGCAACAGCAGGATTGTGCAGTTTTCACCAAG | | |
| | | TGGAAAGTTCATCACACAGTGGGGAGAAGAGTCTTCAGGGACCAGTC | | |
| | | CTCTGCCAGGCCAGTTCACTGTTCCTCACAGCTTGGCTCTTGTGCCT | | |
| | | CTTTTGGGCCAATTATGTGTGGCAGACCGGGAAAATGGTCGGATCCA | | |
| | | GTGTTTTAAAACTGACACCAAAGAATTTGTGAG | | |
| prey2527 | 226 | A | 229 | |
| prey92918 | 228 | ATGAACTCCATGAGTCTCTCCAGGGCTGCCTGCAGCACGTCTTTTCC | 231 | MNSMSLSRAACSTSFPSSLFGFPS |
| | | AAGTAGCCTATTTGGATTCCCATCTCAAATGTCCTGGATGCGAGCGT | | QMSWMRASAAPELGAGEVPFGEPF |
| | | CAGCGGCTCCAGAGCTCGGGGCGGGTGAGGTCCCCTTTGGGGAACCC | | PGHRGRGAAVCGQEDPRGSQERRS |
| | | TTTCCTGGCCATCGAGGTCGGGGGGCTGCCGTCTGTGGGCAGGAGGA | | LHCEKLPGCSAFSFYHGKCRLGPW |
| | | CCCGAGGGGCAGCCAGGAAAGGCGATCTCTTCACTGTGAAAAGTTGC | | GEPAGLWCCPRPPPPPECSSSLGS |
| | | CCGGGTGCAGCGCCTTTTCCTTCTACCATGGGAAATGCAGGCTGGGC | | HKQATQEEQNTASVDFPNFPGK* |
| | | CCTTGGGGTGAGCCTGCGGGGCTCTGGTGCTGTCCCCGACCCCCACC | | |
| | | ACCACCAGAATGCAGTTCCAGCTTACGAACCCACAAACAAGCCACCC | | |
| | | AGGAGGAACAAAACACCGCCAGCGTGGATTTTCCAAATTTCCCTGGA | | |
| | | AAGTAA | | |
| prey102273 | 230 | GGCAACTATCTGCCACTCCAGTGCTATGGGAGCATCGGCTACTGCTG | 233 | GNYLPLQCYGSIGYCWCVFPNGTE |
| | | GTGTGTCTTCCCCAACGGCACGGAGGTCCCCAACACCAGAAGCCGCG | | NPNTRSRGHHNCSESLELEDPSSG |
| | | GGCACCATAACTGCAGTGAGTCACTGGAACTGGAGGACCCGTCTTCT | | LGVTKQDLGPVPM* |
| | | GGGCTGGGTGTGACCAAGCAGGATCTGGGCCCAGTCCCCATGTGA | | |
| prey104937 | 232 | GTTGGTGATAAGGAGGAGACCCTGAATAGTTCTCAGTATTGCCACTC | 235 | VGDKEETLNSSQYCHSGVQQTQNC |
| | | AGGAGTACAACAGACTCAGAACTGCTGGGATGGGTCTGAAACGGTAA | | WDGSETVTIITNIY*TFTMHQELF |
| | | CAATCATAACTAACATATACTGAACATTTACTATGCACCAAGAACTG | | *ERYMAQNKXLA*ILCYSHFTDEK |
| | | TTCTGAGAGCGTTACATGGCTCAAAACAAANCTCTGTGATAGATATT | | AW*LIQCHSDRMHQNQIWTQLIWF |
| | | ATGTTATTCTCATTTTACAGATGAGAAAGCATGGTAACTTATCCAAT | | QSLCH*STTASIVQSTGPCDVREN |
| | | GTCATTCAGACAGAATGCATCAGAACCAGATTTGGACCCAGTTAATT | | S*CQLEXYHY |
| | | TGGTTCCAAAGCTTGTGCCATTAAAGCACTACTGCCTCAATAGTACA | | |
| | | AAGCACTGGTCCCTGCGATGTGAGAGAGAATAGCTGATGTCAGCTAG | | |
| | | AGNTCTACCACTATAA | | |
| prey104938 | 234 | AATGTTCGNGCNTTCTCAACTACTGGCCTTNTGTGTTTTACGGTTTN | 237 | NVRAFSTTGLXCFTVXLRXYVXEX |
| | | ACTTAGGNNNTATGTGGNCGAANNNNTGATTANAGNTTGCTNNTTNG | | XIXXCXXEXXALEHAXXXXNVYIV |
| | | AATGNNTTGCCTTAGAGCACGCTTTNGNATTNNAAAATGTATATATT | | LXCVXDXLTAWPXXXWXSLVVXX* |
| | | GTNCTGCNTTGTGTGGANGACTTNCTCACTGCNTGGCCNNGAANTNA | | GSXXXLVXWLRXXXKATXVIF |
| | | ATGGANGAGTCTTGTAGTTTGNNGGTAGCGNTCTNNTNTCNCNCTNG | | |
| | | TTNGGTGGCTGAGGGNANNAANGAAGGCTACNTNTGTGATATTTC | | |
| prey104941 | 236 | CAGGCTCAAGCGATCCTGCCCCAGCCTCCTGAGCAGCTAANACACAG | 239 | QAQAILPQPPEQLXHRP*HHAQ |
| | | GCCATGACACCATGCCCAGCTANTTTTTGATTTTTTTGTAAAGATTT | | LXFIDFFVKISPLXXXXXXXXXX |
| | | CACCACTNNNNNNNNNNNNNNNNNNNNNNNNNNNNNNNNNNNNNNNN | | XXXXXXXXXXXXXLXVVLIRGK |
| | | NNNNNNNNNNNNNNNNNNNNNNNNNNNNTACTCTNTGTAGTCCTAAT | | ESG*WDRGKSNKRKXXSHKSXF |
| | | TAGGGGAAAGGAGTCAGGCTAGTGGGACCGAGGGAAAAGCAATAAAA | | LHRPGHIXPPV*ITXNLPVPNY |
| | | GAAAGCANATNAGCCACAAGTCANCCTTTCTTCATCGTCCAGGACAC | | XHTPXS*LTATLAFSVLHN |
| | | ATAANTCCTCCTGTGTAAATAACTCANAATCTTCCTGTGCCCAACTA | | |
| | | TAANCACACACCTNCNAGTTAGCTCACTGCAACCTTGGCNTTTTCAG | | |
| | | TACTCCACAAC | | |
| prey69469 | 238 | A | 241 | |
| prey104944 | 240 | GGATACCAAAGGTCCCCAAGTGCTCCGGGGTGTCTTGTGCTCCTGTG | 243 | GYQRSPSAPGCLVLLCLCAHCSLW |
| | | TCTCTGTGCACACTGCTCTCTGTGGCTGGAGGGTACATTTCCCTACC | | LEGTFPYPXCLGNHXLXFQDSAKX |
| | | CATTNTGCTTGGGCAATCACCNACTTNTTTTTCAAGACTCAGCCAAG | | RSCYRASFXLFVDLPVSSTTL*TD |
| | | NGACGATCATGCTACAGAGCCTCATTTNCCTTATTTGTTGACTTGCC | | XEQRLVHSXLAPPVPAQCLALQML |
| | | TGTTTCTTCCACTACACTNTAAACTGATNGGGAACAAAGACTTGTCC | | HAFLLTINCLCGCLGCEVYTEFT* |
| | | ACTCCATNTTGGCACCTCCAGTGCCAGCACAGTGCCTTGCTTTGCAG | | YSFIISSXASRITEVK*LXXKTML |
| | | ATGCTGCATGCATTTTTGTTGACAATAAATTGTTTATGCGGCTGCCT | | EFRGIGNRIFXAPQXAXKRTPW*Q |
| | | GGGCTGTGAGGTGTACACAGAATTTACCTGATACAGCTTCATCATTA | | XTXSFKFLTLTSLYFLLMGKVQLD |
| | | GTTCTNAGGCAAGTAGAATCACAGAAGTAAAATAATTAANTGANAAG | | FHTFSTAVPAAQNSR |
| | | ACTATGTTAGAGTTTAGGGGCATAGGAAATAGGATATTCCNCTGACC | | |
| | | ACAAGANGCATNAAAAAGGACNCCCTGGTAGCAANTTACANGGAGCT | | |
| | | TTAAGTTTTTAACNCTGACTTCACTGTACTTTCTACTCATGGGCAAA | | |
| | | GTGCAATTAGATTTCCATACCTTTAGTACAGCTGTTCCTGCTGCACA | | |
| | | GAATTCCCGC | | |
| prey56846 | 242 | GAAGATTGTAAATGCCAAGAAAGATGTTGTGAACACAAAGATGTTTG | 245 | KIVNAKKDVVNTKMFEELKSRLID |
| | | AGGAGCTCAAGAGCCGTCTGGACACCCTGGCCCAGGAGGTGGCCCTG | | TLAQEVALLKEQQALQTVCLKGTK |
| | | CTGAAGGAGCAGCACGCCCTGCAGACGGTCTGCCTGAAGGGGACCAA | | VHMKCFLAFTQTKTFHEASEDCIS |
| | | GGTGCACATGAAATGCTTTCTGGCCTTCACCCAGACGAAGACCTTCC | | RGGTLSTPQTGSENDALYEYLRQS |
| | | ACGAGGCCAGCGAGGACTGCATCTCGCCCGGGGGCACCCTGAGCACC | | VGNEAEIWLGLNDMAAEGTWVDMT |
| | | CCTCAGACTGGCTCGGAGAACGACGCCCTGTATGAGTACCTGCGCCA | | GARIAYKNWETEITAQPDGGKTEN |
| | | GAGCGTGGGCAACGAGGCCGAGATCTGGCTGGGCCTCAACGACATGG | | CAVLSGAANGKWFDKRCRDQLPYI |
| | | CGGCCGAGGGCACCTGGGTGGACATGACCGGCGCCCGCATCGCCTAC | | CQFGIV* |
| | | AAGAACTGGGAGACTGAGATCACCGCGCAACCCGATGGCGGCAAGAC | | |
| | | CGAGAACTGCGCGGTCCTGTCAGGCGCGGCCAACGGCAAGTGGTTCG | | |
| | | ACAAGCGCTGCCGCGATCAGCTGCCCTACATCTGCCACTTCGGGATC | | |
| | | GTGTAG | | |
| prey104947 | 244 | GCTGGANTGGCNAGNGTGCATNATCNTCCANACNTAGTGCANCTGAT | 247 | AGXAXVHXXAXXVXLITTKFIFXA |
| | | TACTACCAAAGAGATTTTTTANGCNTAATAANNGTTGGGNTCTTTGG | | **XLGSLGXXXFXXHXDAXXNPXH |
| | | GATTNTTNTNTTTTNAGGNTCATNAAGATGCGNGCNNAAACCCATNN | | XSTKPCLXXTXFXX*XXQLSXADX |
| | | CATGNCTCNACNAAGCCATGCTTGGNNNATATTTNCTTTNTTCANTA | | EYXVXASAVXXXXLTWKIXLECIX |
| | | ANGNNTGCAATTATCNCNNGCAGATNNAGAATACNTAGTACNGGCNT | | TGFXMXXYXSGXMXXAGXYGKXWP |
| | | CNGCTGTNNNTTNGGNCNCNCTAACCTGGAAAATTTGNTTGGAGTGC | | LLPGQDX |
| | | ATTNNGACCGGCTTTNTNATGNNACNCTACTANTCAGGAANAATGNN | | |
| | | TANTTGAGGNNAATACGGAAAANNTTGGCCCTTACTTCCTGGNCAGG | | |
| | | ACCNN | | |
| prey104951 | 246 | TTTCATTATGGGTGGCTGTGAGACTTTCTATCTCATCAATCCGTGAA | 249 | FHYGWL*DFLSHQSVKMPEHLYNQ |
| | | AATGCCAGAGCATCTTTATAATCAATCAAATCCTGAATCTTCTGGAA | | SNPESSGTS*LSACQFRKTV*KRG |
| | | CGTCTTAGTTATCAGCTTCTCAATTTCGGAAAACCGTTTAGAAAAGA | | *GYLFQLIGHTNTCLKV**SICGL |
| | | GGTTAGGGCTATTTATTTCAATTAATTGGCCATACCAATACCTGCTT | | MLYIGMFFQFLEDPVAAPPGSRC* |
| | | GAAAGTTTAATAGTCAATTTGTGGGCTAATGCTTTACATACGAATGT | | GCC*HSCLKMIRL*SHNQGVI*FP |
| | | TTTTCCAGTTCCTGGAGGACCCTGTAGCAGCACCACCTGGTTCCAGG | | HQTLGCSKAHGIWL*AEPSDLPQL |
| | | TGCTGAGGCTGCTGTTGACATTCTTGTCTGAAAATGATAAGGTTGTA | | YLLSLPPNFHC*GHLHSAGRCEVQ |
| | | ATCACATAATCAAGGTGTGATTTGATTTCCACATCAGACACTAGGCT | | *CTHSSMSYGVFNF*FCVDNRIPH |
| | | GTTCCAAAGCCCATGGAATTTGGCTGTAGGCAGAACCCAGTGATTTG | | VPGQKGFTKLSPCVVTKHSTICLV |
| | | CCACAATTATATCTTCTGTCTCTTCCTCCAAATTTTCACTGCTAGGG | | SS |
| | | CCACCTTCATTCTGCTGGAAGATGTGAAGTGCAATAGTGCACACACT | | |
| | | CATCAATGAGCTATGGGGTCTTTAACTTTTATTCTGTGTCGACAATA | | |
| | | GAATCCCGCATGTTCCTGGACAGAAAGGGTTCACCAAACTCAGTCCA | | |
| | | TGTGTAGTCACCAAACACAGTACCATATGTTTGGTGAGTAGCTT | | |
| prey104954 | 248 | TTGACTTATTATNATCAATATGTCTAGACCCTTCTGATCTTCCCAAC | 251 | LTYYXQYV*TLLIFPTLPVPFQFS |
| | | TTTACCCGTGCCTTTTCAGTTCTCTTCTTCTCATTCCTCACCACTGA | | SSHSSPLMDSHPSFQEGLKSCCLD |
| | | TGGACAGTCATCCTTCCTTCCAAGAAGGGCTTAAGTCTGGTTGCCTG | | SR*FPSSLAXPPXIVWTPCAMYNY |
| | | GACTCTAGATAATTTCCCTCCTCCTTAGCANTTCCACCTTTNATTGT | | PGIVNCLSMSVTLLLCYRIA1YHG |
| | | CTGGACTCCTTGTGCCATGTACAATTATCCCGGTATTGTTAATTGTC | | TVPCFFTE*VLNILLFNLLELCNF |
| | | TTTCTATGTCTGTAACCTTGCTTCTGTGTTATAGACATTATCATGGC | | IKITLSLKMFPVGI*GP*DGARKG |
| | | ACAGTGCCGTGCTTTTTCACAGAATAGGTACTTAATATTTTATTATT | | DKGFFLSYLFFLCKLHNFQLFPTL |
| | | TAATTTACTAGAACTTTGCAATTTCATAAAGATAACTTTGTCATTAA | | KK*SLAILYDLNIKMIH*FLEKKQ |
| | | AAATGTTTCCGGTGGGGATTTAGGGCCCCTGAGATGGTGCAAGAAAA | | KTKKQKLLSPFQKCKK |
| | | GGAGATAAAGGGTTTTTTTTAAGCTATTTGTTCTTTCTGTGCAAATT | | |
| | | ACATAACTTCCATTATTTCCAACGTTAAAGAAATAGTCCCTNTGAAT | | |
| | | CCTTTATGATTTAAACATTAAAATGATTCACTAGTTTTTAGAAAAAA | | |
| | | AACAAAAAACAAAAAAACAAAAACTGCTGTCACCCTTCCAAAAATGT | | |
| | | AAAAAA | | |
| prey104961 | 250 | GGAAATAAGAAAGGCTTTTCAAAATCCATCTGNNNNNNNNNNNNNNN | 253 | GNKKGFSKSIXXXXXXXXXXXXXX |
| | | NNNNNNNNNNNNNNNNNNNNNNNNNNNNNNNNNNNNNNNNNNNNNNN | | XXXXXXXXXXXXXXXXXXXXXXXX |
| | | NNNNNNNNNNNNNNNNNNNNNNNNNNNNNNNNNNNNNNNNNNNNNNN | | XXXXXXXXIT*XLGHXPXXPXPXX |
| | | NNNNNNNNNNNNNNNNNNNNNNNNNAAATTACCTAGGNNCTGGGGCA | | EXGGSXXPXXFRL*XXXXQPLALX |
| | | TNCCCCTNTANTCCCANCTCCTNAGGNGGAANNGGGNGGATCCNTTG | | PGQXNXIXSFKXXFKKXSX*XGSX |
| | | ANCCCAGNANTTTTAGGCTGTAANAANCTTTNANACAGCCCCTTGCC | | LN |
| | | CTCCNNCCTGGGCAANAAAATNAAATTTTNTCTTTTAAAAANNTNTT | | |
| | | TAAAAAAAANTCCTTNTAACNGGGAAGTTNTTTAAAT | | |
| prey104962 | 252 | GCAGCTTCTAACTCCCAATTTCTAGGTTTTTACTATGGTTTTGTGTA | 255 | AASNSQFLGFYYGFV*HP*DTFPY |
| | | ACATCCCTAGGACACATTTCCCTATTTTTCCTTATGCCACTGTAAAG | | FSLCHCKGSSLILKLLVAFLPNFY |
| | | GTAGCAGCCTCATTCTTAAGTTACTAGTATAATTTCTGCCAAATTTC | | HLLSYIIV**HFAIILTFKDPSFE |
| | | TATCATTTGTTGAGCTACATCATAGTCTAATAACATTTTGCAATTAT | | GIYLAYNRF*M*FRKLTFVPENPL |
| | | CTTAACTTTTAAAGATCCAAGTTTTGAAGGAATTTACTTAGCATACA | | FQMAQWSC*NYVNII*IYPPLCFL |
| | | ATCGCTTTTGAATGTAATTCAGAAAACTTACTTTTGTACCAGAAAAT | | HCHFFLRNEAN*G |
| | | CCATTATTCCAAATGGCACAATGCTCTTGTTAAAACTATGTTAATAT | | |
| | | CATCTAAATTTACCCTCCTCTTTGTTTTTTGCATTGTCATTTCTTCC | | |
| | | TAAGAAATGAGGCAAATTGAGGCT | | |
| prey104964 | 254 | AAGTGGTTCTCTCACTTCAGTCTCCCAAGTAGCTCGGACTACCGNAC | 257 | KWFSHFSLPSSWDYRTCATXVPEX |
| | | NTCTCCCACCNTTGTTCCCGAAAANCGGGACCCTTNGNCCCCTNNNC | | RDPXXPXPLXCXGXXXGXX*RAKX |
| | | CNCTTTNAGGGAANGGGANTTTNANTGGNTNCCNTTAAAGGGCNAAA | | XPPKTXPXGXKXXXPXXRXPPSPR |
| | | NNNANNCCCCCAAAAACTTNNCCCNGGGGCCNNAAANANNNNTTNCC | | VFXEXXGGGXKXGGXXPXFFXXWP |
| | | CCNNNAAAGGNGGCCCCCNTCNCCCCGGGTTTTTNGGGAAAANNNNG | | XPXXGG |
| | | GNGGGGGNNAAAAAAANGGGGGGANNANNCCCNTTTTTTTTTNTNTT | | |
| | | TGGCCCCNCCCCCNNNNGGGGGGGG | | |
| prey104984 | 256 | G | 259 | |
| prey104985 | 258 | TTTCACTTTTCAATGCCCTCANATGNTCCTNTCCATCATTATGATTA | 261 | FHFSMPSXXPXHHYDYYXYFXSLX |
| | | CTATNGTTACTTTTANTCATTGTTNCGNTNNGCCCATGTGTNNACCA | | RXAHVXTNFCLTLAX*IXLXYFAI |
| | | ATTTCTGTCTCACTCTTGCTTNTTGAATTTNCCTTTTNTATTTTGCT | | XXSLGLHLLLXPSEEHPTHGH*NL |
| | | ATAATNTTNTCCCTCGGATTGCACCTTTTGTTANNTCCTTCNGAAGA | | IIKXLAPMP*TLXXAGQVGTVSXA |
| | | ACATCCCACTCATGGGCATTGAAATCTGATTATAAAGNGACTAGCAC | | X*LGTQSXXXTX*RASXFFXXKXQ |
| | | CCATGCCATAGACCCTTNTTCNTGCNGGNCAGGTNGGNACCGTTTCT | | XXXXXXVXXGXG |
| | | NTNGCCANCTAGTTGGGAACTCAATCTNNANTGCNGACATGNTGAAG | | |
| | | AGCCTCTCNTTTCTTTNCTTTNAAGNNGCAANTATTNNATNNTNAAN | | |
| | | ATGTGTANNGGGGNCNGGGAGNGGX | | |
| prey78515 | 260 | A | 263 | |
| prey5244 | 262 | ATGGATACTAAGCTGATGTGTTTGTTGTTCTTTTTCTCCCTGCCTCC | 265 | MDTKLNCLLFFFSLPPLLVSNHTG |
| | | GCTCCTAGTGAGTAACCACACTGGCCGCATCAAGGTGGTCTTTACTC | | RIKVVFTPSICKVTCTKGSCQNSC |
| | | CGAGCATCTGTAAAGTGACCTGCACCAAGGGCAGCTGTCAGAACAGC | | EKGNTTTLISENGHAADTLTATNF |
| | | TGTGAGAAGGGGAACACCACCACTCTCATTAGTGAGAATGGTCATGC | | RVVICHLPCMNGGQCSSRDKCQCP |
| | | TGCCGACACCCTGACGGCCACGAACTTCCGAGTGGTAATTTGCCATC | | PNFTGKLCQIPVHGASVPKLYQHS |
| | | TTCCATGTATGAATGGTGGCCAGTGCAGTTCAAGGGACAAATGTCAG | | QQPGKALGTHVIHSTHTLPLTVTS |
| | | TGCCCTCCAAATTTCACAGGAAAACTTTGTCAGATCCCAGTCCATGG | | QQGVKVKFPPNIVNIHVKHPPEAS |
| | | TGCCAGCGTGCCTAAACTTTATCAGCATTCCCAGCAGCCAGGCAAGG | | VQIHQVSRIDGPTGQKTKEAQPG |
| | | CGTTGGGGACGCATGTCATCCATTCAACACATACCTTGCCTCTGACC | | |
| | | GTGACTAGCCAGCAACGAGTCAAAGTGAAATTTCCTCCTAACATAGT | | |
| | | CAATATCCATGTGAAACATCCTCCTGAAGCTTCCGTCCAGATACATC | | |
| | | AGGTTTCAAGAATTGATGGCCCAACAGGCCAGAAGACAAAAGAAGCT | | |
| | | CAACCAGGCC | | |
| prey104988 | 264 | TTTGTTTTGAGACAGTCTCACTATGGTGTGANGCTGCAGTGCAATGG | 267 | FVLRQSHYGVXLQCNGRSXXXXXX |
| | | CAGGAGTNNNNNNNNNNNNNNNNNNNNNNNNNNNNNNNNNNNNNATC | | XXXXXXXPPASASGVAGSTGTRHH |
| | | CTCCTGCCTCAGCCTCTGGAGTAGCAGGGTCTACAGGCACGCGCCAT | | AWPKNQKAYKVKKLQ*AKVNLLLK |
| | | CATGCCTGGCCAAAAAATCAAAAAGCTTATAAAGTAAAAAAGTTACA | | KEIF*NKCSVA*VYCVYIVDSSAQ |
| | | GTAAGCTAAGGTTAATTTATTATTGAAGAAAGAAATATTTTAAAATA | | QCPRSSHSLTTHSHDSPRATSSLE |
| | | AATGTAGTGTAGCCTAAGTGTACTGCGTTTATATAGTCGACAGTAGT | | NFIHGKCPMQMYHFLIF |
| | | GCACAGCAATGTCCTAGGTCTTCCCATTCACTCACCACTCACTCGCA | | |
| | | TGACTCACCCAGAGCAACTTCCAGCCTTGAAAACTTCATTCATGGTA | | |
| | | AGTGTCCTATGCAGATGTACCATTTTTTGATCTTTTA | | |
| prey104990 | 266 | CTCCCTGGGGGTGGGAAAGCATCCTCCGTGGTTGTTTGTCCCCCACC | 269 | LPGGGKASSVVVCPPPQIPRSTLW |
| | | CCAGATCCCCAGGTCCACCTTGTGGCCTTTTTTTAAGGATGAAGACC | | PFFKDEIDLAEPEAGEVGHSELCM |
| | | TGGCAGAACCTGAGGCCGGGGAGGTGGGCCATTCTGAGTTGTGTATG | | AVGFWVWHSLETLAVL**RKTGVL |
| | | GCTGTCGGTTTCTGGGTCTGGCACTCTCTGGAGACCCTTGCTGTCCT | | PALSNSRDLSGHHSAFPASPL*RG |
| | | TTGATAGAGAAAAACAGGGGTCCTCCCAGCCCTGTCCAACTCCCGGG | | GDSYFLRWQAWRACIYFLLPMAFS |
| | | ACCTCTCGGGCCACCACTCAGCCTTCCCTGCAAGTCCATTGTGAAGG | | L*PGLPLHCSAHLGNS*ASGLP*S |
| | | GGAGGGGATAGCTACTTCTTGCGGTGGCAGGCCTGGAGGGCCTGCAT | | |
| | | CTACTTCCTATTACCCATGGCCTTCAGCTTATAGCCTGCCCTGCCCC | | |
| | | TGCACTGCTCTGCCCACTTAGGGAATTCATGAGCCAGTGGGCTCCCC | | |
| | | TAGTCC | | |
| prey102493 | 268 | CTTCCGGGAGGATTTCCATTATAATGACACAGCTGGGTACTTCATTA | 271 | FREDFHYNDTAGYFIIGGSRYVAG |
| | | TTGGAGGGAGCAGGTATGTGGCTGGCATTGAAGGGTTTTTTGGACCC | | IEGFFGPLKYYRLRSLHPAQIFNP |
| | | CTGAAGTACTATCGCCTTCGCAGTCTGCACCCCGCCCAGATTTTTAA | | LLEKQLAEQIKLYYERCAAVQEIV |
| | | TCCCCTCCTTGAGAAGCAACTTGCTGAACAAATCAAGTTATATTATG | | SVYASAAKHGGERQEACHLHNSYL |
| | | AAAGGTGTGCTGAGGTTCAAGAAATAGTATCTGTGTATGCATCTGCA | | DLQRRYGRPSNCRAFPWEKELKDK |
| | | GCAAAGCACGGGGGCGAGAGACAAGAAGCATCCCACCTCCACAACTC | | HPSLFQALLEMDLLTVPRNQNESV |
| | | CTACCTGGACCTCCAGCGCAGGTATGGGAGACCCTCGATGTGCAGAC | | SEIGGKIFEKAVKRLSSIDGLHQI |
| | | CCTTCCCCTGGGAGAAGGAGCTGAAAGACAAACACCCCAGCTTGTTC | | SSIVPFLTDSSCCGYHKASYYLAV |
| | | CAGGCATTGCTGGAGATGGATCTGCTGACCGTGCCAAGGAACCAAAA | | FYETGLNVPRDQLQGMLYSLVGGQ |
| | | TGAATCTGTATCAGAAATCGGTGGGAAGATATTTGAGAAGGCTGTAA | | GSERLSSMNLGYKHYQGIDNYPLD |
| | | ACAGACTCTCTAGCATTGATGGTCTTCACCAAATTAGCTCTATCGTC | | WELSYAYYSNIATKT |
| | | CCCTTTCTGACGGATTCCAGCTGCTGTGGATACCATAAAGCATCCTA | | |
| | | CTACCTTGCAGTCTTTTATGAGACTGGATTAAATGTTCCTCGGGATC | | |
| | | AGCTGCAGGGCATGTTGTATAGTTTGGTTGGAGGCCAGGGGAGTGAG | | |
| | | AGGCTGTCTTCAATGAATCTTGGGTATAAACACTACCAGGGTATTGA | | |
| | | CAACTACCCCCTGGACTGGGAACTGTCGTATGCCTACTACAGCAACA | | |
| | | TTGCCACCAAGACACC | | |
| prey104991 | 270 | GCAGGCAGAACTGAATTNAGGCTTGGGGATTTACAGGCAGCAGAACA | 273 | AGRTEXRLGDLQAAEQKRTQVLTP |
| | | GAAGAGAACTCAAGTTCTAACCCCAGCTCTGCCTCTGACCATTGACC | | ALPLTIDPRNHLCRGDYCNGHMKL |
| | | CCAGGAACCATCTCTGCAGGGGTGATTATTGTAATGGTCACATGAAA | | NKSKFFEQNKVSYFVEWF*LYKNQ |
| | | TTAAATAAATCAAAATTCTTTGAACAGAATAAAGTGTCATATTTTGT | | *MVHYLSQQPT*ISLNIFRIAGTP |
| | | AGAGTGGTTTTAGCTCTACAAAAACCAGTAAATGGTACACTATCTTT | | YIKTLPTVKTI*EKSSLDQV*WVY |
| | | CTCAACAACCCACATAAATAAGTCTTAACATCTTTAGAATTGCTGGA | | VKAANIY*TVTIKHTXAILGAGIL |
| | | ACTCCCTATATCAAAACTCTTCCGACAGTGAAGACAATCTAAGAGAA | | VHLTFTAVR*VSLFPGK*RGREEL |
| | | ATCCTCACTAGATCAGGTTTGATGGGTCTATGTGAAAGCAGCTAATA | | XDLPKVXKGRIGRLGSKPWQGPHS |
| | | TTTATTAGACAGTTACTATCAAACACACTANTCCTATTCTAGGAGCT | | LYF*DANTTTRNNXIMNITGGATE |
| | | GGTATATTAGTTCATTTAACTTTCACAGCTGTNAGGTGAGTCTCATT | | |
| | | ATTCCCAGGTAAGTAAAGAGGNAGAGAGGAGTTAANTGATTTGCCCA | | |
| | | AGGTTNCTAAAGGCAGGATAGGAAGGCTAGGATCTAAACCCTGGCAC | | |
| | | GGACCTCATAGCCTGTACTTTTGAGATTAGAATACCACTACCAGAAA | | |
| | | TAATAANATCATGAATATAACAGGGGGAGCCACAGAGAA | | |
| prey104993 | 272 | TNGCCAAGTGGATGCATTTGGGAGATTTTTGAGAGGTGGAAAGGGCA | 275 | XPSGCIWEIFERWKGQDIALLCL* |
| | | GGACATAGCTCTTCTTTGTTTGTAAGGGNAAGAGGATTGATGGAAGA | | GXED*WKKISGLGSITQRWELKTK |
| | | AGATTTCTGGCTTGGGTTCCATCACCCAGAGGTGGGAACTGAAGACA | | TQSEGRWRELSC*MLTLKYPTGDI |
| | | AAGACACAGTCCGAGGGGCGGTGGAGGGAACTCAGCTGTTAAATGCT | | LLGSLIKGYGLGTEILI*NAGFGV |
| | | AACTTTAAAATATCCCACTCGAGATATTTTACTGGGCAGTTTAATAA | | MSIQVRIEDM*VDKNSHQEHAQRE |
| | | AGGGATATGGTCTCGGGACAGAAATTTTGATTTAAAATGCTGGTTTT | | EVLGRNPVRGREPVMEPAMELLLK |
| | | GGAGTCATGAGCATCCAGGTGAGGATTGAAGACATGTGAGTAGATAA | | KKETRRVWHHETQGR |
| | | GAACAGCCATCAAGAACATGCTCAGAGAGAAGAGGTGTTAGGGCGGA | | |
| | | ACCCTGTGAGGGGAAGAGAACCAGTAATGGAGCCTGCGATGGAACTG | | |
| | | CTGTTGAAGAAGAAGGAGACCAGGAGAGTGTGGCACCACGAAACCCA | | |
| | | GGGGAGG | | |
| prey104995 | 274 | CTGGCCTTTTCACTTTTTAAGTTTTATGATCACATATTTTAATTTTG | 277 | LAFSLFKFYDHIF*F*RSPVSLFC |
| | | AAGAAGTCCAGTTTCTCTCTTTTGTTTTACATTTAGTACTTTTTGTG | | FTFSTFCVVSKNVCLDQGCECFLL |
| | | TGGTCTCCAAAAATGTTTGCCTAGACCAAGGTTGTGAATGTTTTCTC | | YFVLEVL*L*HLCLGL*TTXN*FS |
| | | CTATATTTTGTTCTAGAAGTTTTATAACTTTAGCATTTATGTTTAGG | | WAGKGPKFFFF*PLSNCSSITCXX |
| | | GTTATGAACCACTATNAATTAATTTTCTTGGGCAGGGAAGGGGCCAA | | TLLTKFXFIKYLFYXNVIXVXM*X |
| | | AGTTCTTTTTTTTTTAACCTTTATCCAACTGTTCCAGCATTACTTGT | | FY*NFXXF |
| | | TNANAAACTCTTCTNACTAAATTTNGTTTTATAAAATATCTTTTTTA | | |
| | | TNGAAATGTTATTTTNGTAANCATGTAANATTTTTATTAAAATTTCT | | |
| | | NANTTTTCAN | | |
| prey102159 | 276 | GAACAGTGTTAGGCAGTTTTTGGGTGAATATAAATCATGGCAAGACA | 279 | NSVRQFLGEYKSWQDNIQTVLFTL |
| | | ACATTCAAACAGTTCTATTTACATTAGTCCAGGCTATGCGTCAGGTT | | VQAMGQVRSQEHVEMLQEITPTLK |
| | | CGAAGTCAAGAACACGTTGAAATGCTCCAGGAAATCACTCCCACCTT | | ELKTQSQSIYNNLVSFASPLVTDA |
| | | GAAAGAACTGAAAACACAAAGTCAGAGTATCTATAATAATTTAGTGA | | TNECSSPTSSATYQPSFAAAVRSN |
| | | GTTTTGCATCACCCTTAGTCACCGATGCAACAAATGAATGTTCGAGT | | TGQKTQPDVMSQNARKLIQKNLAT |
| | | CCAACGTCATCTGCTACTTATCAGCCATCCTTCGCTGCAGCAGTCCG | | SADTPPSTVPGTGKSVACSPKKAV |
| | | GAGTAACACTGGCCAGAAGACTCAGCCTGATGTCATGTCACAGAATG | | RDPKTGKAVQERNSYAVSVWKRVK |
| | | CTAGAAAGCTGATCCAGAAAAATCTTGCTACATCACCTGATACTCCA | | AKLEGRDVDPNRRMSVAEQVDYVI |
| | | CCAAGCACCGTTCCAGGAACTGGCAAGAGTGTTGCTTGTAGTCCTAA | | KEATNLDNLAQLYEGWTAWV* |
| | | AAAGGCAGTCAGAGACCCTAAAACTGGGAAAGCGGTGCAAGAGAGAA | | |
| | | ACTCCTATGCAGTGAGTGTGTGGAAGAGAGTGAAAGCCAAGTTAGAG | | |
| | | GGCCGAGATGTTGATCCGAATAGGAGGATGTCAGTTGCTGAACAGGT | | |
| | | TGACTATGTCATTAAGGAAGCAACTAATCTAGATAACTTGGCTCAGC | | |
| | | TGTATGAAGGTTGGACAGCCTGGGTGTGA | | |
| prey105000 | 278 | GTGGCATACAGAGAGCTTGCATACGATACTCTTACAACAATTACGTA | 281 | VAYRELAYDTLTRITYYTELL*NY |
| | | CTATACTGAACTTCTATGAAATTATTTACCTGGTCCGTAATGTTATT | | LPGP*CYSXQPIPKRANSATLXGK |
| | | CANGGCAACCCATCCCAAAAAGAGCAAATTCTGCAACCCTCANTGGC | | NAAQHCFPKMSLQSFPL*VYNVAK |
| | | AAAAACGCTGCCCAGCATTGCTTCCCAAAAATGTCCTTGCAAAGTTT | | GSKDQKKSXKKIXI*AISKWKKQK |
| | | CCCATTGTAAGTATACAATGTGGCAAAAGGTTCTAAAGATCAGAAAA | | QEHHSPDGAPKSKINSSQITASNG |
| | | AGTCTTTNAAAAAAATTANAATTTAGTGCATATCCAAGTGGAAAAAA | | SHSSWVSETFFLSSNSVPD*SASS |
| | | CAAAAGCAGGAACATCATTCACCTGATGGTGCTCCTAAATCTAAAAT | | FAHLLFVSQM*MR**KVQL*CNLL |
| | | AAATTCTTCACAGATAACAGCCTCCAATGGCAGCCATAGCTCTTGGG | | WLRS |
| | | TAAGTGAAACATTCTTCCTTTCAAGTAACAGTGTACCTGACTGAAGT | | |
| | | GCAAGCAGCTTTGCTCATCTCCTGTTCGTTTCCCAAATGTGAATGAG | | |
| | | ATGATGAAAAGTTCAGCTGTAGTGCAATTTGCTATGGTTAAGGTCTT | | |
| | | T | | |
| prey105001 | 280 | GCCAGTTTTACAACCCCAACCGGAAGTCTGAATTCCAGTATCTGGGA | 283 | ASFTTPTGSLNSSIWDGSEVLRRI |
| | | TGGATCTGAAGTTCTGAGGCGAATTCGAGGACCACTGCTTGCTCAGG | | RGPLLAQEMPTVSVLEYALP*RPP |
| | | AAATGCCTACGGTGTCTGTATTAGAATATGCTTTGCCTTAGAGGCCC | | QGPEDDWDLSERALPRRSTSPIIG |
| | | CCCCAGGGTCCAGAAGATGATTGGGACCTTTCTGAACGAGCATTACC | | SPPVRAVPIGTPPKQMAVPSFTQQ |
| | | AAGGCGGTCAACTTCACCTATCATTGGCAGTCCTCCTGTTAGAGCTG | | ILCPKPVLVRPPMPPRYPAPFGER |
| | | TCCCCATAGGCACCCCACCTAAACAGATGGCTGTACCCAGCTTCACC | | MSPNQLFSVPNSSLLGHPFPPSVP |
| | | CAACAGATTTTGTGTCCGAAGCCTGTCCTTGTTCGGCCCCCAATGCC | | PVLSP |
| | | ACCTCGTTATCCTGCTCCCTTTGGTCAGAGGATGTCTCCCAACCAGC | | |
| | | TTTTCAGTGTCCCGAACTCTTCCCTCCTGGGTCACCCTTTTCCTCCT | | |
| | | AGTGTTCCTCCTGTTCTCAGCCCCCC | | |
| prey105005 | 282 | ACCGGGCAAGCTACCTTACATTANTGGCAGAAAAAAAGTGCAACTGA | 285 | TGQATLHXWQKKSATDILQRDFLS |
| | | CATACTACAAAGAGATTTTTTAAGTTTAAAAAAAGTTTGGATCTTTT | | LKKVWIFWISFFFXGXXGPKKPXK |
| | | GGATTTCTTTTTTTTTTTTNGGNNTTTNNGGNCCTAAAAAACCCNTA | | XNXPXXXXXXFFFFXXXXPKXRXG |
| | | AAANANAATNGCCCCCNCAANTANNGNNGGNTTTTTTTTTTTTTTTN | | VFSFXPXSRFXAGXXPPXGPPLXL |
| | | GGNCNNACNCCCAAAANNCCGGNTGGGGGTTTTTTCCTTTNCCCCCC | | XPXGGRXXKTXQXXXXGPKGGXXG |
| | | NTTCCCGATTTTNNGCNGGNNCCCNTCCCCCNNNGGGCCCCCCCCTN | | XXGPLLXXKKKXPXKKPPFFFK |
| | | ANTTTANCCCCCNNGGGGGGGCGANTTTNTAAAACNNCCCAAAANNA | | |
| | | AANANCGGGCCCNAAGGGGGGAANNNGGGGAAANCNTGGTCCCCTTT | | |
| | | TGNNTTNGAAAAAAAAANCCCCCNCAAAAAAACCCCCNTTTTTTTTT | | |
| | | AAAT | | |
| prey78563 | 284 | G | 287 | |
| prey27857 | 286 | ATGGCCCTCCCTTATCATATTTTTCTCTTTACTGTTCTTTTACCCTC | 289 | MALPYHIFLFTVLLPSFTLTAPPP |
| | | TTTCACTCTCACTGCACCCCCTCCATGCCGCTGTATGACCAGTAGCT | | CRCMTSSSPYQEFLWRMQRPGNID |
| | | CCCCTTACCAAGAGTTTCTATGGAGAATGCAGCGTCCCGGAAATATT | | APSYRSLSKGTPTFTAHTHMPR |
| | | GATGCCCCATCGTATAGCAGTCTTTCTAAGGGAACCCCCACCTTCAC | | |
| | | TGCCCACACCCATATGCCCCGCA | | |
| prey78469 | 288 | CCTGAAGATGGTCCCTCAGCCAGGCAGCCTCTGCCCAGAAGGAACGT | 291 | PEDGPSARQPLPRRNVPVGITEGD |
| | | GCCGGTTGGCATCACTGAAGGAGATGCCTCAAGGACTGGGAGTCTCC | | GSRTGSLPASSVQLLQD*CTAG |
| | | CAGCAAGTTCTGTGCAACTTTTGCAAGA | | |
| prey105019 | 290 | GCTAGGACCACAAGGCATGAGCCACCGAGNTCCAGCCNTGCTTTATT | 293 | ARTTRHEPPXSSXALFFSYSTGNL |
| | | TTTCTCTTACAGTACTGGGAATCTTCTAATATACTAAGGACACGTGT | | LIY*GHVYFLSVFPRTGA*CGQVF |
| | | ATTTTCTTTCTGTCTTCCCTAGAACAGGAGCTTAATGTGGGCAGGTA | | LLISFITLSPVPGQGLAHEWCVLN |
| | | TTTTTGTTGATCTCATTTATCACCCTATCCCCAGTGCCTGGACAGGG | | KYF**INKSNILQEKAISGTHPPT |
| | | TCTGGCACATGAATGGTGTGTTCTAAATAAATATTTTTAATAGATAA | | EPKSAKTFSAVSPY*THPTPGEPV |
| | | ATAAATCAAATATCCTACAAGAGAAAGCTATATCTGGAACTCACCCA | | XGKRXDKGGLREQVVPTK |
| | | CCAACAGAACCGAAAAGCGCAAAGACCTTTAGCGCGGTCTCTCCCTA | | |
| | | CTGAACACACCCAACCCCGGGGGAGCCAGTANGAGGAAAAAGAANGG | | |
| | | ACAAAGGTGGGCTAAGGGAGCAGGTGGTGCCCACCAAGT | | |
| prey105021 | 292 | GATGGGCCNGCCCTTCTGGGCTGTAGTTGGGCTTTTTTAGGATATTT | 295 | DGPALLGCSWAFLGYF*GSPVHPL |
| | | CTAGGGCAGTCCAGTACATCCCTTAGTGGTNATCATATATGGGCATG | | VVIIYGHEDVLKTXXLFREGRCHX |
| | | AGGATGTTTTGAAAACTCANAANCTCTTTAGAGAAGGGAGGTGCCAT | | KHX*VESCSRTRWVVXLVLP*VFG |
| | | ATNAAACATCNTTAAGTGGAGAGTTGTTCACGAACTAGGTGGGTNGT | | CFL*ATD*XPLG*G*GLHSWVPVL |
| | | TNCTTTGGTCTTGCCATAGGTTTTTGGCTGTTTCTTATAAGCTACTG | | XXAHEEXXRXXFXXPGXLGXEXXX |
| | | ATTGAANACCCCTGGGGTAGGGGTGAGGCCTGCATTCTTGGGTCCCN | | XRGXKPRPP |
| | | GTTCTAANANTGGCCCACGAGGAANTGAGNCGNGNTNTTTTTCNTTN | | |
| | | CCCCGGGCNCTTGGGTTNNTTCNNNANGTNGNAAAGGGGGNAAAAGC | | |
| | | CGCGNCCTCCCC | | |
| prey78379 | 294 | AGCGGCATCCCCTACGGTCAGCCCAAGCAGGAAAATGATTTCAATCC | 297 | SGTPYGQPKQENDFNPDECQPYEL |
| | | TGATGAATGCCAGCCCTATGAGCTCCCAGATGGCTCAGTCGTCATCA | | PDGSVVINARNQNNYHCHCRIVLR |
| | | ATGCCCGAAACCAGAACAACTACCACTGCCACTGCCGAATTGTCCTC | | SYDACDTLRPRDVTFDPELVDPVV |
| | | CGCAGCTATGATGCCTGTGATACACTAAGGCCCCGTGATGTGACCTT | | AAGAVVTSSGIVFFSNPAHPEFRV |
| | | CGACCCTGAGCTCGTGGACCCTGTGGTAGCTGCAGGAGCTCTAGTCA | | NLTLRWSFSNGTSWRKETVQLWPG |
| | | CCAGCTCCGGCATTGTCTTCTTCTCCAACCCAGCACATCCAGAGTTC | | PSGYSSLATLEGSMDGEEQAPQLY |
| | | CGAGTGAACCTGACCCTGCGATGGAGCTTCAGCAATGGTACCTCATG | | VLYEKGRNHYTESISVAKISVYGT |
| | | GCGGAAAGAGACAGTCCAGCTATGGCCAGGCCCCAGTGGCTATTCAT | | L* |
| | | CCCTGGCAACCCTGGAGGGCAGCATGGATGGAGAGGAGCAGGCCCCC | | |
| | | CAGCTCTACGTCCTGTATGAGAAAGGCCGGAACCACTACACAGAGAG | | |
| | | CATCTCCGTGGCCAAAATCAGTGTCTATGGGACACTCTGA | | |
| prey105022 | 296 | TTTTTAACTTTTTTGTAGAGATTGGGTTTGGCCATATTAACCGNNNN | 299 | FLTFLARLGLAILTXXXXXXXXXX |
| | | NNNNNNNNNNNNNNNNNNNNNNNNNNNNNNNAAGACATTAGCTTGGT | | XXKTLAWSLLKLADPYFCLIISNL |
| | | CTCTTCTCAAATTAGCAGATCCTTACTTCTGTCTTATAATTTCTAAT | | LL*LLTYIQSSSFSCLFQLDKPPY |
| | | CTGCTTTTATAATTATTGACTTACATTCAATCTTCCAGTTTTTCATG | | LYLTSSKILTMLFHF*LKFKRT |
| | | TCTCTTCCAATTAGATAAGCCTCCATATTTGTATTTAACTTCCAGTA | | |
| | | AAATCTTAACCATGTTGTTTCATTTTTAATTAAAATTTAAGAGGACG | | |
| prey105029 | 298 | GAACTGTTTATGTTTACCCGTTCTACATTNAAGGATGCCCTTTTACT | 301 | ELFMFTRSTXKDALLLYMCFLEYY |
| | | ATATATGTGCTTTCTAGAATATTACTGATTCTTACATATCTTTAAAA | | *FLHIFKSWIFVIA*RTGNTYFV* |
| | | GTTGGATATTTGTAATAGCTTAAAGGACAGGTAACACTTATTTTGTG | | SFLI*KQKINEQVSSNDLL*DFSV |
| | | TAATCTTTCTTAATTTAAAAACAAAAAATAAATGAACAAGTCAGTTC | | LESCYTCRTGVXGSRLQSQHPGRA |
| | | CAATGATCTCCTTTGAGATTTTTCAGTTTTAGAAAGTTGCTACACTT | | KAGRITXRSRARDQXRANMGEAPV |
| | | GTCGGACAGGTGTGGNNGGCTCACGCCTGCAATCCCAGCACCCAGGG | | SXKKHKK |
| | | AGGGCCAAGGCAGGGCGGATCACCTNAAGGTCAAGAGCTCGAGACCA | | |
| | | GNCCCGGGCCAACATGGGGGAAGCCCCCGTCTCCNCTAAAAAACACA | | |
| | | AAAAAT | | |
| prey105031 | 300 | TGTGTGTGTGTGTGTGTGNACAAAAAACATTATNTGTTGTGGTGGTT | 303 | CVCVCVXKKHYXLWWLCVLXLILE |
| | | GTGTGTTCTTIAIGTTTAATACTGGAAAAAGAAAAANACCAAAANCT | | KEKXQXLAPRGIFPXXXKXXGXXX |
| | | TGCNCCCCGNGGGATTTTTCCCNNTTTNNAAAAANCCNCNGGGGNAA | | LXFFXXKKTXIFXPKKKKFWXKXX |
| | | ANNTTTTAANTTTTTTCCNNNGAAAAAAAACNANAATTTTTNNCCCA | | XXGGXXPXFFFGXIFIXXPXFXGP |
| | | AAAAAAAAAAAATTTTGGGNAAAAANGGNANGAGNGGGGGGGCNCNC | | XFFXFFKKKKSXXPPPXXKXILXG |
| | | CCCCCNTTTTTTTTTTGGNNNNATTTTTATTTTNANCCCGCNTTTTT | | GGEX |
| | | NGGGNCCCCNTTTTTTCNNTTTTTTTAAAAAAAAAAAATCTNTTTTN | | |
| | | CCCCCCCCGNGNNNNAAANAAATTCTGNGGGGCGGGGGGGAANNTTT | | |
| prey102321 | 302 | AAAAAAGAAAGAAAGAAAGAAAGAGAAATCAGCGGTAAGGTAAGCAG | 305 | KKEARKKEREISGKVSRTQRS*YS |
| | | GACCCAGAGGAGCTGATATTCACAGTTCTTACATGGACAACTCTTTC | | QFLHGQLFQELSIKYFILQPAFLV |
| | | AGGAATTATCCATAAAGTACTTTATTTTACAACCTGCTTTTCTTGTA | | KLKVHFFT*KFYTVLKPELCVKYC |
| | | AAACTAAAGGTGCACTTTTTTACATAAAAGTTTTATACAGTGTTAAA | | MYMFLNSLVPVVHR*LLWLRHHFP |
| | | ACCAGAACTGTGTGTAAAATACTGCATGTATATGTTTCTAAATAGTC | | WMILKESLQFYWNWILLILLFHGS |
| | | TTGTTCCAGTGCTTCATCGGTGACTTCTGTGGCTTCGTCATCATTTT | | QKNHYFQGLRFHYLQNVVNLSQLE |
| | | CCATGGATGATTCTGAAAGAATCTCTCCAGTTTTACTGGAATTGGAT | | THQIPPFHYFPQKSLHSQLRHHQI |
| | | CCTACTAATTCTTCTGTTTCACGGCAGTCAGAAGAACCACTACTTTC | | LFLVYFFLGSFLLMS*KTLKLSKC |
| | | AGGGCCTTCGTTTTCACTACCTTCAGAATGTAGTAAATCTTTCTCAG | | CCT*ILFSKYLHS*VS*ILMLCSS |
| | | CTTGAGACACATCAGATTCCTCCATTTCATTATTTTCCTCAGAAGTC | | NHIP*NSAATAYRWEKSPASALQG |
| | | TCTTCATTCACAGTTGAGGCATCATCAGATTCTTTTTCTTGTTTATT | | VP*RTVLSRKQ*MQEFEDLLTPPL |
| | | TCTTTCTGGGATCATTTCTCTTGATGTCATAAAAGACTCTAAAACTA | | LWCEAM*CLSVMSAERA*FGEIRT |
| | | AGCAAATGTTGTTGTACTTAAATTTTGTTTTCAAAATACCTCCACAG | | LRGQSVQKCGGIGRAGENNEPRIQ |
| | | TTAAGTTTCATGAATTCTGATGCTCTGTAGTTCAAATCACATCCCCT | | DFKK*ENVQLLKPGEESPRAGILG |
| | | GAAATTCAGCAGCAACTGCATACAGGTGGGAGAAAAGCCCAGCGTCG | | QRCPKCRLAKWGWGKGPGFDWVEL |
| | | GCATTACAAGGAGTTCCATGACGTACAGTTCTTTCACGAAAACAATG | | EVQGRAGVSSVYPQWYLVSICSRG |
| | | AATGCAAGAATTTGAGGATCTCCTTACTCCTCCCTTATTGTGGTGTG | | RCD*WGPRVSPVLQQP*TMDPKSC |
| | | AAGCAATGTGATGTCTGAGTGTGATGAGCGCTGAGAGAGCATGATTT | | QCGPCYPHHLAPADRGSAGRVSGA |
| | | GGGGAAATCAGAACTTTGAGAGCTCAGTCAGTTCAGAAGTGTGGAGG | | FPLLPGRGQRCPHVCIHHGCRPSL |
| | | AATAGGCAGAGCCGGTCAAAATAACGAGCCTAGGATCCAAGATTTTA | | LLLPHVLVRAQCCQPLRGCAGCVH |
| | | AAAAGTAGGAAAATGTCCAGTTACTGAAGCCAGGGGAGGAGAGCCCC | | ASLPEVSGCPFPGLHLLPPSTPC* |
| | | AGGGAAGGGATACTTGGACAGCGTTGTCCAAAATGTACGCTGGCAAA | | VCGTACRVDCPQGCSVAVGLPEAQ |
| | | GTGGGGATGGGGCAAAGGCCCTGGGTTTGATTGGGTGGAACTAGAGG | | TAGGPYPMKKPPPSLHLLVLGPRE |
| | | TCCAGGGTAGAGCTGGGGTATCCTCTGTCTACCCTCAGTGGTATCTT | | LKGVGQGGV*RLFLGLRPPKYAPP |
| | | GTGTCCATATGTTCTAGGGGTAGATGTGATTAATGGGGCCCTCGAGT | | Q*LRFPA*ELGRERSNPFKEVITL |
| | | CAGTCCTGTCCTCCAGCAGCCGTGAACAATGGACCCCAAGTCATGTC | | EW*QEEQEARPALVLHPHVFQVEQ |
| | | AGTGTGGCCCCTGCTACCCTCACCATCTTGCACCAGCAGACAGAGGC | | EELVQARPHPPGPSRGRRRKGLVQ |
| | | AGTGCTGGGAGAGTGTCGGGTGCGTTTCCTCTCCTTCCTGGCCGTGG | | AWVPSPCSMGTSAVLISLIKSVCT |
| | | GCAGAGATGTCCACACGTTTGCATTCATCATGGCTGCCGGCCCAGCC | | |
| | | TCCTTCTGCTGCCACATGTTCTGGTGCGAGCCCAATGCTGCCAGCCT | | |
| | | CTCAGAGGCTGTGCAGGCTGCGTGCATGCTTCGCTACCAGAAGTGTC | | |
| | | TGGATGCCCGTTCCCAGGCCTCCACCTCCTGCCTCCCAGCACCCCCT | | |
| | | GCTGAGTCTGTGGCACGGCGTGTAGGGTGGACTGTCCGCAGGGCTGT | | |
| | | TCAGTCGCTGTGGGGCTCCCTGAAGCCCAAACGGCTGGGGGCCCATA | | |
| | | CCCCATGAAGAAGCCCCCACCTTCCCTCCACCTGCTTGTGTTGGGCC | | |
| | | CCAGGGAACTAAAGGGTGTGGGTCAGGGAGGGGTCTAGAGGCTATTC | | |
| | | CTAGGCCTCAGGCCTCCCAAATATGCCCCTCCCCAGTAGCTACGGTT | | |
| | | CCCTGCCTAGGAGCTGGGGAGGGAGAGATCTAATCCCTTCAAGGAAG | | |
| | | TGATAACACTGGAGTGGTAACAAGAGGAGCAGGAAGCAAGGCCGGCC | | |
| | | CTGGTTCTCCATCCCCATCTGTTTCAGGTGGAACAGGAGGAACTGGT | | |
| | | CCAGGCCAGGCCTCATCCTCCTGGACCCAGCAGGGGCAGAAGGAGGA | | |
| | | AGGGACTGGTCCAGGCATGGGTCCCTTCCCCCTGCTCCATGGGCACC | | |
| | | TCTGCTGTATTGATATCACTAATAAAGTCTGTCTGCACTGC | | |
| prey105032 | 304 | AAATATTCAGGAAAAAAAATGGATGGTTGCATCTGTACTAAACATGT | 307 | KYSGKKNDGCICTKHVKQYGTTTI |
| | | TAAACAATATGGTATAACAACTATTTGCATAGCATTTACCCTGTATT | | CIAFTLYSVL*LTYR*FKVYRRRC |
| | | CAGTATTATAGCTAATCTATAGATGATTTAAAGTATATAGAAGGAGG | | VGYMQIVYHLW*GT*PSIDFSMSG |
| | | TGCGTAGGTTATATGCAAATAGTATACCATCTGTGGTAAGGAACTTG | | QSWPQSLTDTEDDCIYKLYINF*H |
| | | ACCATCCATAGATTTTAGTATGAGCGGGCAGTCCTGGCCCCAGTCTC | | F*CYGLKFFF*A*RLCRRYQERLC |
| | | TCACAGACACCGAGGATGACTGTATATATAAACTATATATAAACTTT | | RHSVYKY*Y*T |
| | | TAACATTTTTAATGTTACGGTCTTAAATTTTTTTTTTGAGCATAGAG | | |
| | | GCTCTGCAGAAGATATCAAGAGAGGCTATGTAGGCATTCAGTCTATA | | |
| | | AATATTAATATTAAACACC | | |
| prey102459 | 306 | ATGCAGCGCTACAATTTTCATTATCTTAAGTACATTGTACATTTCTA | 309 | MQRYNFHYLKYIVHFYRTCDYSRM |
| | | CAGAACCTGTGATTATTCTCGCATGATAAGGATGGTACTTGCATATG | | IRMVLAYGELLLLTVSAEILFQWT |
| | | GTGAATTACTACTGTTGACAGTTTCCGCAGAAATCCTATTTCAGTGG | | NIVAWQQMPTFCGIAANLQETLVG |
| | | ACCAACATTGTGGCATGGCAGCAAATGCCAACATTTTGTGGAATAGC | | FSFCFLCFFPLLLNQQGWKEGREV |
| | | AGCAAATCTACAAGAGACCCTGGTTGGTTTTTCGTTTTGTTTTCTTT | | MNYSFQ* |
| | | GTTTTTTCCCCCTTCTCCTGAATCAGCAGGGATGGAAGGAGGGTAGG | | |
| | | GAAGTTATGAATTACTCCTTCCAGTAG | | |
| prey107650 | 308 | A | 311 | |
| prey92681 | 310 | 6 | 313 | |
| prey107662 | 312 | TATATCCTCTAAAAAACAGGAGTAGTGGCTCATGCCTGTAATCTCAG | 315 | YIL*KTGVVAHACNLSIFGGLGRW |
| | | CATTTTCGGAGGCTTAGGTAGCTGGATCCGTCGAGCCCAGAAGTTTC | | IRRAQKFQTWAT*RNLVSPEKKKX |
| | | AGACCTGGGCAACATAACGAAACCTCGTCTCTCCAAAAAAAAAAAAA | | KKXKXXXXXKKKKXFXPXXGGPXX |
| | | NGNAAAAAAANNAAANNAAANNAANNAAANAAAAAAAGAANTTTTNC | | XPXXXGXXGGXGFXTPXXXGXXXP |
| | | CCCNGGGNNGGGGGNCCNNNCNTTTNACCCCNNNNTNTTGGGGNCCN | | XPPPXXXXFXKKGXXFXKKXKKKK |
| | | GGGGGGGGNGGGTTTTTTNACCCCNNNAANNNNNGGNTTNANGNAAC | | XGXXGX |
| | | CNNNACCCCCCCCTTNNCNNNCNNCNTTTNNNAAAAAGGGNANCNTT | | |
| | | TTTTNAAAAAAANGAAAAAAAAAAANGGGNTNNNGGGGGGNC | | |
| prey107665 | 314 | TCTTCACCAAGACAGAGACGTTAGCAACGCATGGCGGCTGGCGACCT | 317 | SSPRQRR*QRMAGGDLGCSGGGTR |
| | | GGGGTGCTCAGGAGGCGGTACCCGGGGCCCCGGCCAGAGATACATCA | | GPGQRYTNYTPVGGQPMGASTSRI |
| | | ATTACACCCCCGTGGGGGGACAGCCGATGGGAGCCAGCACCAGCAGG | | RGRPGQRSAPPTSSPPPVPQRAGP |
| | | ATCCGAGGGCGCCCCCGACAGAGGTCTGCCCCACCCACTTCCTCCCC | | AREGGVLESSVPAANRFWVFPERV |
| | | ACCACCTGTGCCCCAGAGAGCAGGGCCTGCCCGGGAAGGTGGCGTCC | | *VTPHSCDPTGLCPTYTEVANEWC |
| | | TGGAGTCGAGTGTACCTGCAGCCATGAGGTTCTGGGTGTTTTTTGAG | | ICATWGAR |
| | | AGAGTCTGAGTGACACCACACTCGTGTGACCCCACAGGGTTGTGTCC | | |
| | | AACATACACGGAAGTGGCTATGGAATGGTGTATTTGTGCAACCTGGG | | |
| | | GTGCGCGN | | |
| prey107667 | 316 | CTGTTGTGCAGCTGGGTTTGAGAAACATTAGATAAAACTCTTGGAAA | 319 | LLCSWV*ETLDKTLGKFTSIIKKN |
| | | ATTTACAAGTATTATTAAAAAAAACCAGCATATAACAATAACATCAA | | QHITITSMNTYIVVCVSYYVFTY* |
| | | TGAACACTTATATAGTAGTATGTGTCAGTTATTATGTATTCACATAT | | CLY*IKGVSVKTKI*LSGFYPDSS |
| | | TGATGTCTTTATTGAATTAAAGGGGTCAGTGTAAAAACAAAAATATA | | IDFL*QRRYSNLFSSTGHEYKATT |
| | | ACTATCTGGGTTCTATCCAGATTCTTCCATAGATTTCTTGTAACAAA | | TCPLCRTLQESLWATRVTQVVLV* |
| | | GGAGATATAGCAATTTGTTTTCATCAACTGGACACGAATACAAAGCT | | G*KDYQ**QQFQWT |
| | | ACAACTACTTGTCCCTTATGCAGGACCTTACACGAATCTTTATGGGC | | |
| | | CACACGTGTGACTCAAGTTCTACTGGTCTAACGGTAAAAAGACTATC | | |
| | | AATAGTGACAACAATTTCAATGGACC | | |
| prey107681 | 318 | ATGGCTGCTTTGTTAGGTGGAGTACATCAGTGTCCTTTGGGCATTCC | 321 | MAALLGCVHQCPLGIPHAKCKMVV |
| | | TCATGCCAAAGGAAAGATGGTAGTTAGTATTGCTGAAGATCTTTTAC | | SIAEDLLRTAAQNSRLSLQRTQAG |
| | | GAACTGCTGCCCAAAATAGCAGGCTATCTTTACAGCGCACCCAACCT | | WLLLGALMTLGPSVVRYHLPKMLL |
| | | GGCTGGCTTTTACTTGGAGCACTTATCACTTTACGACCATCTGTCGT | | LWRNVFPRSLKELEAEKARGDSFT |
| | | TCGTTACCATCTGCCCAAGATGTTCTTATTGTCCCGAAATGTTTTCC | | WQVTLECRAGALCAMRSFVAHCPE |
| | | CACGTTCCTTAAAGGAATTGGAAGCTGAGAAGGCCCGAGGCGATTCT | | LLTEDVIRKLMTPIECAMTMMSHI |
| | | TTTACCTGGCAGGTAACTTTGGAAGGTCGTGCTGGAGCTCTATGTGC | | PSVMKA |
| | | CATGAGGAGCTTCGTTGCACATTGTCCTGAGCTACTAACTGAAGATG | | |
| | | TGATTCGAAAATTGATGACCCCTATTGAATGTGCCATGACTATGATG | | |
| | | TCACACATTCCATCTGTAATCAAAGCCCA | | |
| prey107678 | 320 | T | 323 | |
| prey107692 | 322 | ATTTATTGTGTTTTGTAGTCGTTTGGAATTGTTCCTCTCTCTGCCTT | 325 | IYCVL*SFGIVPLSALCFKKLISF |
| | | ATCTTTTAAAAAGCTAATATCTTTTGTATTCTACAACCTGTCTTCAC | | VF*NLSSQILF*VYQILSLFFFHC |
| | | ACATACTTTTCTAAGTTTACCAAATCTTAAGCCTATTTTTCTTTCAT | | FSFHYINSTL*YKKLKVITPSLVM |
| | | TGCTTTTCTTTTCATTATATAAACTCAACCCTCTAATATAAAAAACT | | ALSYSFPS*LDFL*CEPLYLYFLA |
| | | AAAAGTTATCACACCCAGTCTGGTTATGGCCTTATCTTACTCCTTTC | | SQ*YLQLLQPGFFLHFTKLMLLRW |
| | | CTTCATAGCTGGACTTCTTATAATGTGAACCGTTATATCTATATTTT | | LIIS*LLHL |
| | | CTTGCTTCCCAATAATATCTTCACTTATTGCAGCCTGGTTTCTTCCT | | |
| | | CCACTTCACAAAACTGATGTTGCTAAGATGGCTAATCATCTCTTAGT | | |
| | | TACTGCATTTAT | | |
| prey107848 | 324 | CAATAATTTACTTTTAAAGGCACAGAGTTATACTGGAGATATGGAAC | 327 | NNLLLKAQSYTGDMEELCWVAVTG |
| | | AGCTGTGTTGGGTATAAGTAACAGGCTTTTCTTTATCTTCTATGTGG | | FSLSSMWLFANCSPGTLHTLNSLI |
| | | CTCTTTGCAATGTCCTCACCTGGGACCCTTCACACACTTAATTCACT | | HFLQIVFWSVCFCYIYMSRKKLQL |
| | | TATACATTTTTTACAAATTGTATTTTGGTCAGTATGTTTTTGTTACA | | VVFCHVIFLR*SVKFYRLDL*NIF |
| | | TTTATATGTCCAGGAAGAAATTACAGCTTGTGGTATTTTGCCATGTC | | I*V*QLKCCVFIVSFSYLFSFCPR |
| | | ATCTTCCTTAGGTAGTCTGTGAAATTTTATAGGTTAGATTTGTAAAA | | TLARAEHKRFFSKSDTEKI*KNWT |
| | | TATATTTATCTGAGTCTACCAGTTGAAGTGTTGTGTTTTTATTGTCT | | WQFTVKKRM*KCG*V*GAKKRL*W |
| | | CTTTCAGTTATTTGTTCTCATTTTGCCCAAGAACTTTGCCTAGAGCA | | A*PMLDNIYPLQNISM**ICESLS |
| | | GAACATAAAACATTCTTTTCAAAAAGTCATACTGAGAAGATATAAAA | | S |
| | | AATCTGGACATGGCAATTTACAGTTAAAAAAAGGATCTGAAAGTGTG | | |
| | | GATGACTGTAAGGTGCAAAAAAGAGGTTATAATGCGCTTAACCAATG | | |
| | | CTTGACAACTACCCATTCCAAAATATTTCAATGTCATAAATATGTGA | | |
| | | AAGTCTTTCATCA | | |
| prey107700 | 326 | AGACTTACTCACTGTCACAAGAACNGCATGGGAAAGACCTGCCCCCA | 329 | RLTHCHKNXMGKTCPHD*LPAYWX |
| | | TGATTAATTACCTGCTTACTGGGNCCCTCCCATANCACATGGGAATT | | PPIXHGNSR*DLXGDTAKPYQS*Y |
| | | CAAGATGAGATTTGGNTGGGGACACAGCCAAACCNTATCAGTCATAA | | ILLFKKYLNICFL*XTXNLFLGGV |
| | | TATATCCTCCTCTTTAAAAAATATTTAAATATTTGCTTTTTATAGNC | | ADRDPILNFFQIHNQLYXYCLLNT |
| | | TACTTANAACTTATTTTTGGCNGGGGTATGAGATAGAGATCCAATTT | | VLSPVICSGTSETHXISXMHCSXS |
| | | TAAATTTTTTCCAAATTCATAACCAATTGTACCANTATTGTTTATTG | | GCFMYLMII*FL |
| | | AATACAGTCCTTTCCCCAGTGATCTGCAGTGGTACCTCTGAAACACA | | |
| | | TCANATTTCACANATGCATGGATCAGNTTCTGGTTGCTTCATGTACC | | |
| | | TAATGATAATTTAATTTCTT | | |
| prey107702 | 328 | N | 331 | |
| prey1080 | 330 | A | 333 | |
| prey78388 | 332 | A | 335 | |
| prey107740 | 334 | TGCTTGTTCTCATTCNGACCTGTTGNATTAAATATTTCAACTTCTGT | 337 | CLFSFXPVXLNISTSVIKKGXYIL |
| | | TATAAAGAAAGGTCANTATATACTATTCCACCTGTCATCCTAAATTA | | FHLSS*IMHKIFKYLTHLTSLLPA |
| | | TGCACAAAATATTCAAATATCTTACTCATTTAACTTCATTGCTTCCA | | VQTRAR*GNQS*R*NCHQRRSSSY |
| | | GCCGTTCAAACCAGGGCCAGATGAGGTAATCAATCATAGAGATAGAA | | *SEPEH*TA*KYFLLVHVVRLTRN |
| | | TTGCCACCAAAGAAGGTCGTCTTCTTATTAGTCAGAACCTGAACATT | | AEGK*VEQVREAVFTPMYPSWVFF |
| | | AAACAGCATAAAAGTATTTCTTACTTGTGCATGTAGTAAGACTCACT | | *SKSLI |
| | | AGAAATGCTGAAGGGAAATAAGTAGAGCAGGTGAGAGAAGCAGTTTT | | |
| | | TACTCCGATGTATCCAAGTTGGGTTTTTTTCTGATCAAAAAGCTTAA | | |
| | | TTAT | | |
| prey107745 | 336 | T | 339 | |
| prey78271 | 338 | G | 341 | |
| prey107747 | 340 | GGTTGTACAGTTCTGTGCATTTGCTAAAAACTATTGAATTGCACACC | 343 | GCTVLCIC*KLLNCTPAKKLLVER |
| | | TGCAAAAAAGCTATTAGTGGAAAGAAGGAAATTTTTTTTAACAAAGA | | RKFFLTKMPCCMPPGES*VVGTET |
| | | TGCCATGTTGTATGCCTCCTGGAGAATCCTAGGTGGTAGGGACTGAG | | CL*PCD*DGNHILLIHGGLQRIPQ |
| | | ACTTGCTTATAGCCATGTCACTGAGATGGCAACCATATCCTTTTAAT | | LGQQLDCNPMIDFDPEVPS*AVPR |
| | | ACATGGAGGCCTTCAGAGGATCCCACAGCTTGGCCAGCAGCTTGACT | | FLAYGNYEVINVHCFKLLSFGLTC |
| | | GCAACCCCATGATAGACTTTGACCCAGAAGTACCCAGCTAAGCTGTG | | VQQ*TLISSSVAMC |
| | | CCTAGATTTCTGGCCTATGGTAACTATGAGGTAATAAATGTCCATTG | | |
| | | TTTTAAACTACTGAGTTTTGGGTTAACTTGTGTGCAACAGTAAACAC | | |
| | | TCATAAGCAGCAGTGTTCCAATGTGT | | |
| prey95593 | 342 | CATAGAGAAGATCTCCGCCCGGGAGATCCTGGACTCCCGCGGGAACC | 345 | IEKIWAREILDSRGNPTVEVDLYT |
| | | CCACAGTGGAGGTGGATCTCTATACTGCCAAAGGTCTTTTCCGGGCT | | AKGLFRAAVPSGASTGIYEALELR |
| | | GCAGTGCCCAGTCGAGCCTCTACGGGCATCTATGAGGCCCTGGAGCT | | DGDKQRYLGKGVLKAVDHINSTIA |
| | | GAGGGATGGAGACAAACAGCGTTACTTAGGCAAAGGTGTCCTGAAGG | | PALISSGLSVVEQEKLDNLMLELD |
| | | CAGTGGACCACATCAACTCCACCATCGCGCCAGCCCTCATCAGCTCA | | GTENKSKFGANAILGVSLAVCKAG |
| | | GGTCTCTCTGTGGTGGAGCAAGAGAAACTGGACAACCTGATGCTGGA | | AAERELPLYRHIAQLAGNSDLILP |
| | | GTTGGATGGGACTGAGAACAAATCCAACTTTGGGCCCAATGCCATCC | | VPAFNVINGGSHAGNKLAMQEFMT |
| | | TGGGTGTGTCTCTGGCCGTGTGTAAGGCAGGGGCAGCTGAGCGGGAA | | LPVGAESFRDAMRLGAEVYHTLKG |
| | | CTGCCCCTGTATCGCCACATTGCTCACCTGGCCGGGAACTCAGACCT | | VIKDKYGKDATNVGDEGGFAPNIL |
| | | CATCCTGCCTGTGCCGGCCTTCAACGTGATCAATGGTGGCTCTCATC | | ENSEALELVKEAIDKAGYTEKIVI |
| | | CTGGCAACAAGCTGGCCATGCAGGAGTTCATGATCCTCCCAGTGGCA | | GMDVAASEFYRDGKYDLDFKSPTD |
| | | GCTGAGAGCTTTCGGGATGCCATGCGACTAGGTGCAGAGGTCTACCA | | PSRYITGDQLGALYQDFVRDYPVV |
| | | TACACTCAAGGGAGTCATCAACGACAAATACGGCAAGGATGCCACCA | | SIEDPFDQDDWAAWSKFTANVGIQ |
| | | ATGTGGGGGATGAAGGTCCCTTTGCCCCCAATATCCTGGAGAACAGT | | IVCDDLTVTNPKRIERAVEEKACN |
| | | GAAGCCTTGGAGCTGGTGAAGGAAGCCATCGACAAGGCTGGCTACAC | | CLLLKVNQIGSVTEAIQACKLAQE |
| | | GGAAAAGATCGTTATTGGCATCCATGTTGCTGCCTCAGAGTTTTATC | | NGWGVMVSHRSGETEDTFIADLVV |
| | | GTGATGGCAAATATGACTTGGACTTCAAGTCTCCCACTGATCCTTCC | | GLCTGQIKTGAPCRSERLAKYNQL |
| | | CCATACATCACTGGGGACCAGCTGGGGGCACTCTACCAGGACTTTGT | | MRIEEELGDEARFAGHNFRNPSVL |
| | | CAGGGACTATCCTCTCGTCTCCATTGAGGACCCATTTGACCAGGATG | | * |
| | | ATTGGGCTGCCTGGTCCAACTTCACAGCCAATGTAGGGATCCAGATT | | |
| | | GTGGGTGATGACCTGACAGTCACCAACCCAAAACGTATTGAGCGGGC | | |
| | | AGTGGAAGAAAAGGCCTGCAACTGTCTGCTGCTCAAGGTCAACCAGA | | |
| | | TCGGCTCGGTCACTGAAGCCATCCAAGCGTGCAAGCTGGCCCAGGAG | | |
| | | AATGGCTGGGGGGTCATCGTGAGTCATCGCTCAGCAGAGACTGAGGA | | |
| | | CACATTCATTGCTGACCTGGTGGTGGGGCTGTGCACAGGCCAGATCA | | |
| | | AGACTGGTGCCCCGTGCCGTTCTGAACGTCTGGCTAAATACAACCAC | | |
| | | CTCATGAGAATTGAGGAAGAGCTGGCGCATGAAGCTCGCTTTCCCGG | | |
| | | ACATAACTTCCGTAATCCCAGTGTGCTGTGA | | |
| prey107763 | 344 | GAAATCCAGGGTCACAGGCATTGTGCAGGAGAAGCTGCTGAATGACT | 347 | KSRVTGIVQEKLLNDYLNRIFSSS |
| | | ACCTGAACCGCATCTTTTCCTCTTCTGAACATGCACCCCCAGCAGCC | | EHAPPAATSRKPLNFQNLPEHLDQ |
| | | ACCAGCAGGAAACCCCTGAACTTCCAGAACCTGCCAGAACATTTGGA | | LLQVDNEEAESQGQVEGRLGPSTV |
| | | CCAGTTGCTACAGGTGGACAATGACGAGGAGGAAAGCCAGGGACAGG | | VLDHTGGFEGLLLVDDDLLGVIGH |
| | | TTGAAGGGCGGCTTGGCCCATCCACTGTGGTCCTGGACCACACAGGC | | SNFGTIRSTTCVYKGKWLYEVLIS |
| | | GGCTTTGAGGGGCTTCTCCTGGTGGATGATGACCTGCTGGGGGTGAT | | LPACILIGGSPAMALIVPELDFQS |
| | | TGGACACAGCAACTTTGGCACCATCCGCTCTACCACATGCGTGTACA | | GPQ* |
| | | AAGGGAAATGGCTCTACGAGGTCCTCATCTCCTTGCCTGCCTGTATC | | |
| | | CTCATTGGTGGGAGCCCAGCCATGGCCCTAATTGTGCCTGAGCTTGA | | |
| | | CTTTCAGTCAGGGCCACAGTGA | | |
| prey107767 | 346 | CAACAGTACCATGCAACCTTCTCACCATCACCCAACCACTTCAGCCT | 349 | NSTMQPSHHHPTTSASHSHGGGDS |
| | | CACACTCCCATGGTGGAGGAGACAGCAGCATGATGATGATGCCTATG | | SMMMMPMTFYFGFKNVELLFSGLV |
| | | ACCTTCTACTTTGGCTTTAAGAATGTGGAACTACTGTTTTCCGGTTT | | INTAGEMAGAFVAVFLLAMFYEGL |
| | | GGTGATCAATACAGCTGGAGAAATGGCTGGAGCTTTTGTGGCAGTGT | | KIARESLLRKSQVSIRYNSMPVPG |
| | | TTTTACTAGCAATGTTCTATGAAGGACTCAAGATAGCCCGAGAGAGC | | PNGTILMETHKTVGQQMLSFPHLL |
| | | CTGCTGCGTAAGTCACAAGTCAGCATTCGCTACAATTCCATGCCTGT | | QTVLHIIQVVISYFLMLIFMTYNG |
| | | CCCAGGACCAAATGGAACCATCCTTATGGAGACACACAAAACTGTTG | | YLCIAVAAGAGTGYFLFSWKKAVV |
| | | GGCAACAGATGCTGAGCTTTCCTCACCTCCTGCAAACAGTGCTGCAC | | VDITEHCH* |
| | | ATCATCCAGGTGGTCATAAGCTACTTCCTCATGCTCATCTTCATGAC | | |
| | | CTACAACGGGTACCTCTGCATTGCAGTAGCAGCAGGGGCCGGTACAG | | |
| | | GATACTTCCTCTTCAGCTGGAAGAAGGCAGTGCTAGTGGATATCACA | | |
| | | GAGCATTGCCATTGA | | |
| prey107826 | 348 | GCTGTGCTGCATACAAACATTGTATTAGTAAGACCACGGACCACTGA | 351 | AVLHTNIVLVRPRTTEVFPMQRFY |
| | | AGTCTTTCCAATGCAGAGATTCTATGGTTCAATGTGAGATACAGGAA | | GSM*DTGNLKKRKRKKEKKXLXSY |
| | | ACTTAAAAAAAAGAAAAACAAAAAAAGAAAAAAAAANCCTTTNTTCC | | YXAKXSN*XLXQFXKNILKGSKNX |
| | | TATTATAANGCAAAANGAAGTAATTAANATTTANAACAATTTNTTAA | | XF*YXIKTPKYFAH*ISMPGIMKX |
| | | AAACATTTTAAAAGGTTCTAAAAACTNTATNTTTTAATACNCCATTA | | AFVFNSWXXRKIL*FFFFFFFFFX |
| | | AAACACCCAAATATTTTGCACATTAAATTAGTATGCCAGGAATTATG | | KXXXXXPXX |
| | | AAANGNGCTTTTGTATTTAATTCTTGGNCAGNGAGGAAAATATTATA | | |
| | | ATTCTTTTTTTTTTTTTTTTTTTTTTTTNAAAAAAANTNCCNTNNNN | | |
| | | NCCCCCNNNCGGG | | |
| prey107782 | 350 | GGGAAAAAAAAGGGGGTGGACAAGGAAGGTGCANCCTACTGGGCCTG | 353 | GKKKGVDKEGAXYWAWEEAGEGSD |
| | | GGAGGAGGCTGGTGAGCGGTCGGATGATGGGAAATGCTTTCGAGGCC | | DGKCFRGQLEAPMLPLGWFAAETD |
| | | AGCTGGAGCCCATGCTCCCCTTAGGATGGTTCTAGGCAGAAACAGAC | | RTLNSCLETLTCF*FCSCASM*S* |
| | | AGGACACTGAACAGCTGTTTGGAGACATTGACATGTTTCTGATTTTG | | GILAVCGKLSSSVXXXXXXXXXXX |
| | | CAGTTGTGCTTCTATGTGAAGTTAAGGTATAGCAGTATGTGGGAAAC | | XXXXXXXXWHDRPLTVXLXLLGSS |
| | | TGTCATCTTCAGTNNNNNNNNNNNNNNNNNNNNNNNNNNNNNNNNNN | | DPPTSXSXIIXXNPXPX |
| | | NNNNNNNNNNNNNNNNNNNNNNNGTGGCATGATCGCCCGCTTACTGT | | |
| | | ANCCCTTNACCTNCTGGGATCAAGTGATCCTCCCACCTCANCCTCCT | | |
| | | NAATACNCTNGAACCCGCNGCCCGNN | | |
| prey107784 | 352 | N | 355 | |
| prey107799 | 354 | GAGAATGAGAAAGTGAACATGTACTGTGTATCTGATGACCAATTGAT | 357 | ENEKVNMYCVSDDQLICALCKLVG |
| | | CTGTGCCTTATGCAAACTGGTGGGTCGTCACCGAGACCATCAGGTCG | | RHRDHQVASLNDRFEKLKQTLEMN |
| | | CATCCCTGAATGATCGATTTGAGAAACTCAAGCAAACTCTGGAGATG | | LTNLVKRNSELENQMAKLIQTCQQ |
| | | AACCTCACCAACCTGGTTAAGCGCAACAGCGAACTAGAAAAATCAAA | | VEVCNRNICDFSEDLKWVTLWEDF |
| | | TGGCCAAACTAATACAGATCTGCCAGCAGGTTGAGGTATGTAACAGA | | SFVRV*PSHLE*RIEWQF*KVSFL |
| | | AACATTTGTGATTTTTCAGAGGACCTGAAATGGGTGACCCTTTGGGA | | *DACSKKKCQKP |
| | | GGACTTCAGTTTTGTCAGAGTGTAGCCTAGTCATTTGGAATGAAGGA | | |
| | | TTGAATGGCAGTTTTAGAAAGTCTCATTCCTTTAGGATGCTTGTTCC | | |
| | | AAAAAAAAGTGTCAGAAACCTAT | | |
| prey1469 | 356 | TGAACTCAAGAGTGGAGAATACTGGGTTGACCCTAACCAAGGATGCA | 359 | ELKSGEYWVDPNQGCKLDAIKVFC |
| | | AATTGGATGCTATCAAGGTATTCTGTAATATGGAAACTGGGGAAACA | | NMETGETCISANPLNVPRKHWWTD |
| | | TGCATAAGTGCCAATCCTTTGAATGTTCCACGGAAACACTGGTGGAC | | SSAEKKHVWFGESMDGGFQFSYGN |
| | | AGATTCTAGTGCTGAGAAGAAACACGTTTGGTTTGGAGAGTCCATGG | | PELPEDVLDVQLAFLRLLSSRASQ |
| | | ATGGTGGTTTTCAGTTTAGCTACGGCAATCCTGAACTTCCTGAAGAT | | NITYHCKNSIAYMDQASGNVKKAL |
| | | GTCCTTGATGTGCAGCTGGCATTCCTTCGACTTCTCTCCAGCCGAGC | | KLMGSNEGEFKAEGNSKFTYTVLE |
| | | TTCCCAGAACATCACATATCACTGCAAAAATAGCATTGCATACATGG | | DGCTKHTGEWSKTVFEYRTRKAVR |
| | | ATCAGGCCAGTGGAAATGTAAAGAAGGCCCTGAAGCTGATGGGGTCA | | LPIVDTAPYDIGGPDQEFGVDVGP |
| | | AATGAAGGTGAATTCAAGGCTGAAGGAAATAGCAAATTCACCTACAC | | VCFLA |
| | | AGTTCTGGAGGATGGTTGCACGAAACACACTGGGGAATGGAGCAAAA | | |
| | | CAGTCTTTCAATATCGAACACGCAAGGCTGTGAGACTACCTATTGTA | | |
| | | GATATTGCACCCTATGACATTGGTGGTCCTGATCAAGAATTTGGTGT | | |
| | | GGACGTTGGCCCTGTTTGCTTTTTATAA | | |
| prey107812 | 358 | NNNNNNNNNNNNNNNNNNNNNNNNNNNNNNNNNNNNNNNNNNNNNNN | 361 | XXXXXXXXXXXXXXXXXXXXXXXX |
| | | NNNNNNNNNNNNNNNNNNNNNNNNNNNNNNNCACCTTGGTCTGGGAT | | XXHLGLGLQA*ATVPGQHSIILKA |
| | | TACAGGCGTGAGCCACTGTGCCCGGCCAACATTCCATCATTTTAAAA | | GQKGKELVEKGRFYGQNIRMLVFS |
| | | GCTGCGCAAAAAGGGAAGGAACTAGTTGAGAAAGGAAGGTTCTATGG | | FCKHW*NNSWLFCKAFPVGNCKYF |
| | | CCAGAACATCAGAATGCTTGTTTTTAGTTTTTGCAAACATTGGTGAA | | FFFFFSXXXXXXFPXXGGXGGXXX |
| | | ATAATAGTTGGCTTTTTTGTAAAGCCTTTCCAGTTGGAAACTGCAAG | | XXAXXPPKXXPXXP |
| | | TACTTTTTTTTTTTTTTTTTTTCCNAAAANGNNNTCNCNTTNTTTCC | | |
| | | CCNANNTGGAGGNNCGCGGGGNAANNTTTNNTNNNNNGCCNCTTTNC | | |
| | | CCCCAAAANTNNACCCATTNNCCCCCT | | |
| prey107814 | 360 | TTTCTAAAGTCGCAGTATGTGTTACGGGAATTCAGAAGAGAGGAAAA | 363 | FLKSQYVLREFRREEKIGWEWFKK |
| | | AATTGGCTGGGAATGGTTTAAAAAGGAAAATGAACTTGAAGTAGGTC | | ENELEVGLENRQKEEEGNYLIRGE |
| | | TTGAGAATAGGCAGAAGGAGGAGGAGGGAAATTATTTGATAAGAGGA | | AKIAIGKFLGLF*VRQDQFDRSR* |
| | | GAAGCTAAACATGGGAAATTTCTAGGCCTGTTTTAAGTCAGGCAAGA | | FGKVNNLKSLG*NQIFEGPEYQNC |
| | | TCAGTTTGACAGAAGCAGGTGATTTGGAAAGGTGAATAATCTGAAAA | | DGKCHSGKGL*AKDGCDISTS*I* |
| | | GTTTAGGTTAAAATCAGATTTTTGAAGGCCCTGAATACCAGAATTGT | | W*YHILHTSYV |
| | | GATGGAAAATGCCATTCTGGTAAAGGTTTATAAGCAAAGGATGGATG | | |
| | | TGATATAAGCACATCTTAAATATGATGGTGATACCACATACTTCATA | | |
| | | CCTCATATGTGAC | | |
| prey107816 | 362 | TCTGTCACCCAGGCTGGAGTGCANTGGCATGACCTTGGCTACTGTAG | 365 | SVTQAGVXWHDLGYCSLDFLGSRD |
| | | CCTTGACTTTCTGGGCTCACGTGATCCTCCCACCTCGNCCTCCNGGG | | PPTSXSXVAGTACAHHHALLICLF |
| | | TAGCTGGGACTGCNTGTGCACACCACCATGCCTTATTAATTTGTCTA | | FVQXMFHYIAKNGLKLLGSSTPPS |
| | | TTTTTTGTACAGAGNATCTTTCACTATATTGCCAAGAATGGTCTCAA | | TSASRGAGITGMSCCAWLRFLLLX |
| | | ACTCCTGGGCTCAAGCACTCCTCCTTCCACCTCGGCCTCCCGNGGTG | | XXKIFNIPXSRXV**AIKYPSNRF |
| | | CCGGAATTACAGGCATGAGCTGCTGTGCCTGGCTGAGGTTTTTATTA | | NNYQHLPYLLPF |
| | | TTATNATNATNAAAAATTTTCAATATACCTNACAGTAGACNAGTTTA | | |
| | | ATGAGCTATCAAATACCCCTCAAATAGGTTTAACAACTATCAACATT | | |
| | | TGCCATATTTACTCCCCTTTC | | |
| prey107817 | 364 | CACAACTGGTCTCTTTTAGCTTTTTGAAGATGANATAACTTTTCATT | 367 | HNWSLLAF*R*XNFSLIIIILLGK |
| | | AATTATTATTATTCTACTGGGAAAAAAAAACAAAAACTTATTTCCCC | | KNKNLFPLSNXNTIPVKXPFPILL |
| | | TATCTAATNATAACACAATACCTGTGAAGCANCCTTTTCCCATCCTC | | LQSLVTPVVLFASINPFFQXPQMS |
| | | CTCCTTCAGTCTCTGGTAACCCCTGTTGTACTCTTTGCTTCTATCAA | | XIIRS*XICVSLCGLFYLTWYAQG |
| | | CCCTTTTTTTCAGGNTCCTCAAATCAGTGANATAATAAGATCATAAA | | SSVLF*XTEFYPXLWLNSIXXVXX |
| | | GNATTTGIGTTTCTCTATGTGCGTTATTTTACTTAACATGGTATGCT | | YXXLL |
| | | CAAGGCTCATCCCTGCTCTTTTAACNGACACAATTTTATCCTTTNTT | | |
| | | ATGGCTGAATAGTATTNCGNGTGTGNATATNTACNACANTCTCCTT | | |
| prey107822 | 366 | TGCCTTATATTTTATGGCTTTTTTAATCACAGACNTTGTCAAACATA | 369 | CLIFYGFFNHRXCQTYEGXTTXXQ |
| | | CGAAGGANAAACAACGCANAANCAAATCCCTTGAACCCNTCACCCAA | | IP*TXHPISXNPQHFPPLVXLXLY |
| | | TTTCANCAAATCCTCAACACTTTCCTCCTCTTGTCCNTTTACNATTG | | STSTFXWTVRSETR*SFXLICFPT |
| | | TACTCAACAAGCACTTTCNACTGGACCGTCAGGAGCGAGACAAGGTC | | ESSXAASVAXGARPHLASSXDASX |
| | | ATCCTTCTNCTTGATTTGTTTTCCCACCGAGAGCTCTNCGGCTCCGT | | KSSEIXXSXXFPFFYTXTXGALRD |
| | | CTGTCGCCNGTCGGGCTCGCCCTCACCTCGCCTCTTCNNGTGATGCT | | XNRPGSSCGSLS |
| | | TCAATGAAGTCATCAGAAATCTNANTATCTNTGANGTTTCCCTTTTT | | |
| | | CTACACCANAACANGTGGGGCTTTGAGAGACANAAACCGTCCTGGCT | | |
| | | CTTCCTGTGGGTCCCTCTCTGG | | |
| prey107823 | 368 | GTCCTTCCTACATGGATAAGTGTCTTGGCAACGTTAATGGGGAAAAT | 371 | VLPTWISVLATLMGKIVGESAERS |
| | | TGTTGGAGAGAGCGCGGAGAGGTCNTCTTTAAGGAAGATGAAGGGAT | | SLRKMKGYLGFFSVLFKVLSISEY |
| | | ATCTTGGTTTTTTTTCGGTTTTGTTTAAAGTACTGTCAATTTCTGAA | | IEL*IFR*CISTLFTKF*KPTEYY |
| | | TACATAGAGCTTTAAATTTTTCGATAGTGCATTTCAACGCTCTTCAT | | FVANEIMLRPTPLMGGGARTGILR |
| | | AAAATTTTAAAAGCCCACTGAATACTACTTTGTAGCAAATGAGATAA | | IFKGIDFSSXXRLAISKDLGMNGH |
| | | TGTTAAGACCTACCCCTCTNATGGGAGGTGGGCCGAGGACCGGAATA | | LEKFILALXY |
| | | TTAAGGATCTTTAAAGGAATCGACTTCAGCTCTNCANAAAGATTAGC | | |
| | | AATCAGTAAAGATTTGGGGATGAATGGGCACCTAGAGAAGTTTATTG | | |
| | | TTGCTCTCTNTTATT | | |
| prey107828 | 370 | TTTTTTAAATGTGAATTTCTAGCATATTTATAAAAATATAACTACTT | 373 | FFKCEFLAYL*KYNYFYKEWDNIF |
| | | TTATAAGGAATGGGATAACATCTTTTCTGCATGTTATCCAACTTTCA | | SACYPTFT*KNTF*TR*LLVHFIY |
| | | TTTAGAAAAATACTTTCTAAACAAGGTAGTTGTTAGTACATTTTATT | | WVSVIMFYTLK**KLLYINLERYS |
| | | TACTGGGTAAGTGTCATAATGTTTTACACATTAAAATGATAAAAATT | | G*CL*NALFSLTDFSYLYKCYIEF |
| | | ATTGTATATTAATCTTGAAAGGTATTCTGGTTAGTGCCTTTAAAATG | | YKRTKFTFR*ILVRKIVFSIGFSV |
| | | CTCTCTTTTCACTAACAGATTTTAGTTATCTGTACAAATGTTATATT | | IYLFF |
| | | GAATTTTACAAGAGAACCAAATTTACTTTCAGGTAAATTTTGGTGAG | | |
| | | GAAAATTGTCTTTAGCATTGGATTTTCTGTCATCTATTTGTTCTTCA | | |
| | | T | | |
| prey107832 | 372 | GGCCACAGACCTGATCATCTGGACAAGTCGGTTCTCGCCTGGCTGTA | 375 | GHRPDDLDKSVLAWL**P*VVPLK |
| | | GTAGCCCTAGGTGGTACCTTTAAAATATCAAGGGCTGCCACTTGATG | | YQGLPLDGLRAEQFSSSLSAGLLM |
| | | GCCTTAGGTAAGAACAATTCTCCAGTAGCCTCAGTGCCGGCTTGCTC | | SVSCGH*LPGSTSILSECYS*LSS |
| | | ATGTCAGTCTCCTGTGGCCACTGACTTCCAGGTTCTACTTCCATCCT | | GPCHWIAGLLLWPKQQPAMFAGPQ |
| | | CTCTGAGTGTTATAGTTGACTGTCTTCTGGGCCTTGCCACTGGATTG | | GTFQGWIWPRRQM*EQVRNKFNSR |
| | | CTGGTTTATTACTTTGGCCAAAGCAGCAACCTGCCATGTTTGCAGGT | | SAETNTCPKALPGSCQGRGCLRW* |
| | | CCCCAAGGAACCTTCCAGGGATGGATTTGGCCTCGAAGGCAGATGTA | | I*VA |
| | | AGAACAAGTCAGGAACAAGTTCAATAGCAGAAGTGCTGAAACAAATA | | |
| | | CCTGTCCAAAGGCCTTACCTGGATCCTGCCAGGGACGAGGTTGTCTG | | |
| | | AGGTGGTGAATATGAGTTGCT | | |
| prey31081 | 374 | A | 377 | |
| prey107854 | 376 | TGGNTTAGGTACTTANNNGGTGCTANAAGATNNAACTNCATGGTNGC | 379 | WXRYLXGAXRXNXMVACGLT*SLG |
| | | CTGTGGCCTTACTTGATACTCACTAGGATNCCTTNGNNNGGATNNAA | | XLXXDXTXXXSFLVNXXVGSIXTF |
| | | CACNATNTNNATCCTTTCTTGTCAATAANANAGTTGGGTCAATTNNT | | SLFYIIATXTLLCYLSNLTGAGLP |
| | | ACTTTCTCGTTATTTTACATAATAGCTACCNNNACNCTGCTNTGTTA | | GSPG*R*AWXQEXPXPEDXWXPXD |
| | | CCTCTCTAACTTGACTGGAGCTGGACTCCCTGGCAGTCCTCGATAGA | | *SSXFFLKRFLWLPHKXDTLARVA |
| | | GATGAGCCTGGNGGCAGGAANGGCCGGNACCTGAAGACNACTGGCNG | | XTAMG |
| | | CCACNGGACTAGAGCTCCNCCTTCTTTTTGAAAAGATTTCTCTGGCT | | |
| | | NCCACACAAANNTGATACCTTGGCCCGGGTGGCANCAACATGAATGG | | |
| | | GGAT | | |
| prey107855 | 378 | TTGGCACTGTCTACCAGGGTAAATTTGAGTGTGAAGGAAATGCANAA | 381 | LALSTRVNLSVKEMXKRNCTILLN |
| | | AAGGAATTCCACAATTCTTCTCAATTCTGGATTTGCTGGCTTTAATT | | SGFAGFNLNTGFYGLIFLLSSLWD |
| | | TAAATACTGGTTTCTATGGACTAATTTTTCTCCTATCTTCTCTATGG | | SKGLVN*FPGNILRKFTEVLEI*I |
| | | GATTCAAAAGGCTTGGTTAACTGATTTCCAGGGAATATTCTTAGAAG | | PFY*KKK***LKSHILTVYRDCST |
| | | TTCACAGAAGTTCTTGAGATCTAAATTCCTTTCTACTAAAAAAAAAT | | KGIKCLLN*KQYQQQKSK*TKVKI |
| | | AATAATAACTTAAATCACACATTTTGACTCTATATAGAGATTGTTCA | | LFSSLKEVDPIFLQGSKNAFLNIN |
| | | ACTAAAGGAATAAAATGTCTATTAAACTAAAAACAGTACCAACAACA | | SFSQYKNALANVTHFHNWTEITIS |
| | | AAAAAGCAAATAGACAAAAGTTAAAATTTTATTTTCTTCTCTTAAAG | | LPLPCLDLE |
| | | AAGTCGATCCTATTTTTTTGCAAGGATCTAAAAATGCTTTTCTAAAT | | |
| | | ATCAATTCATTCTCACAATATAAAAATGCTCTAGCAAATGTCACCCA | | |
| | | TTTTCATAACTGGACAGAGATAACCATTTCCCTGCCCCTGCCTTGCC | | |
| | | TAGATTTAGAACT | | |
| prey107861 | 380 | T | 383 | |
| prey72968 | 382 | CCGGTCTTGCAAAGTCTCCTGGGATTGGAAGGACCCCTATGAGGTGG | 385 | RSCKVSWDWKDPYEVGHSMAFRVH |
| | | GCCACAGCATGGCCTTCCGAGTGCATTTATTCTATAAGAACGGGCAG | | LFYKNGQPFPAHRPVGLRVHISHV |
| | | CCTTTCCCTGCACATCGGCCTGTGGGACTAAGAGTTCACATCTCTCA | | ELAVEIPVTQEVLQEPNSNVVKVA |
| | | TGTCGAGCTAGCAGTGGAAATTCCAGTGACCCAGGAAGTCCTTCAGG | | FTVRKAGRYEITVKLGGLNVAYSP |
| | | AGCCCAATTCCAACGTAGTAAAAGTGGCCTTCACTGTGCGCAAGGCT | | YYKIFQPGMVVPSKTKIVCHFSTL |
| | | GGGCGTTATGAAATCACAGTGAAGCTTGGTGGATTAAATGTGGCATA | | VLTCGQP |
| | | TAGTCCCTACTACAAAATTTTTCAACCTGGAATGGTGGTTCCTTCTA | | |
| | | AGACCAAAATTGTGTGCCACTTTTCTACTCTTGTATTGACCTGTGGG | | |
| | | CAGCCG | | |
| prey107865 | 384 | GCAATTGCTTCCTTTGCTAAAGAATGTTGCTGTCCTGTGCCGACACC | 387 | GIASFAKECCCPVPTPLGWF*KFP |
| | | ATTGGGGTGGTTTTGAAAGTTCCCCACTGCGTTTGCCATCCCTGCAG | | TAFAIPAAKCFCQRNNLFPD*IFK |
| | | CTAAATGCTTTTGCCAAAGAAATAATTTATTTCCTGATTAAATATTT | | IL*F*IIPKLQIF*IFKLFYFQAA |
| | | AAGATTCTATAATTCTAAATTATACCAAAACTACAAATATTTTGAAT | | ESNYRCHHIHLFSHLFMHSYNKYL |
| | | TTTTAAATTGTTCTACTTCCAAGCAGCTGAAAGTAATTACAGATGTC | | LSIYYVPNIVLGTGDTAVIKTDKN |
| | | ACCACATTCATTTATTCAGTCACTTATTTATGCATTCATATAATAAA | | PCL*EALS |
| | | TACTTATTGAGCATCTATTATGTGCCAAACATTGTTCTAGGCACAGG | | |
| | | GGATACAGCAGTGATCAAAACAGACAAAAATCCCTGCCTTTAAGAAG | | |
| | | CGTTGACCAG | | |
| prey107866 | 386 | TCATTAACCGCAGATNATACAAATTCTATTTAATTTTATAATCCATT | 389 | SLTADXTNSI*FYNAFIVKLHYNT |
| | | TATAGTAAAACTTCATTATAACACTTTGTTCCTTTTAATGTTGCCAA | | LFLLNLPTSNVKTPNCYKKVHCMW |
| | | CATCAAATGTAAAAACACCAAACTGTTATAAGAAGGTCCACTGTATG | | HMLYKLMCF*GCTGTNEGICLLWN |
| | | TGGCACATGCTTTATAAGCTAATGTGTTTTTAAGGATCCACAGGAAC | | MMGML*NSQT*EFHLLKIKVLQTM |
| | | AAACGAGCGAATATGCCTCCTATGGAACATGATGGGGATGTTATAGA | | KHPNLLIKIPVVLLQGCSLEVKTE |
| | | ACAGTCAAACATAAGAATTTCATCTGCTGAAAATAAAAGTGCTCCAG | | NLFREGKRNTDWNCWNKWLSNRGT |
| | | ACAATGAAACATCCAAATCTGCTAATCAAGATACCTGTAGTCCTTTT | | R |
| | | GCAGGGATGCTCTTTGGAGGTGAAGATCGAGAACTTATTCAGAGAAG | | |
| | | GAAAGAGAAATACAGACTGGAACTGTTGCAACAAATCGCTGAGCAAC | | |
| | | AGAGGAACAAGAGC | | |
| prey107869 | 388 | CTGTATGGGCTTTTCTAATATCCAGTTCAAGTGTTTATCAGTGAGTT | 391 | LYGLF*YPVQVFISEFFF*IXLGQ |
| | | TTTCTTTTAAATANATTTGGGACAGGTACTATGAGAGTATATAAGTG | | VL*EYISDTL*DTN*YPMRMAKTA |
| | | ATACGTTATAGGACACTAACTAGTATCCTATGAGGATGGCAAAAACT | | XTFAPTK*KLIRALAYFSTCSLGF |
| | | GCANTCACTTTTGCACCAACCAAATAGAAACTAATCAGGGCACTTGC | | *MSTYCGIXFNPXGILLLFFPGIC |
| | | TTATTTTTCTACATGCTCTTTAGGGTTTTAAATGTCAACCTACTGTG | | *GIEIDWEVSERFCVATGRTCSNN |
| | | GCATANACTTTAATCCTNTGGGTATTCTTTTGTTGTTCTTTCCTGGT | | TWVIS*PKS |
| | | ATATGCTGAGGAATTGAGATAGACTGGTTCGTGAGCGAGAGATTTTG | | |
| | | TGTTGCCACAGGTAGGACATGCTCAAACAATACTTCGGTCATTTCTT | | |
| | | GACCCAAGTCAT | | |
| prey107871 | 390 | GGATGGGAACACAGCCAAACAATATCAATGTTATATGGCTTTTTTTA | 393 | GWEHSQTISMLYGFF*RSFVSTPH |
| | | AAGGTCTTTTGTCTCTACCCCACATCTGAAATGTATTGTTTAAATTG | | LKCIV*IVYFAGI*VFLFLYYVVF |
| | | TTTATTTTGCTGGTATTTAAGTATTCTTGTTTTTATATTATGTTGTG | | TDS*DFVGKV*TSGKIG**TALRA |
| | | TTTACAGATTCTTAGGATTTTGTTGGAAAAGTGTAAACATCAGGAAA | | AFYFVTVSDFSCVHKILLA |
| | | AATTGGTTGATAAACAGCTTTGAGGGCAGCTTTCTATTTTGTAACTG | | |
| | | TCTCTGATTTTAGCTGTGTACATAAAATTTTATTGGCGGC | | |
| prey107875 | 392 | G | 395 | |
| prey107876 | 394 | ACCCTGGCCCTGGTCCCTCCCTCTGCCTGCCTGGTCTGCCTGCAGTG | 397 | TLALVPPSACLVCLQCPTCAKCFL |
| | | CCCCACCTGTGCCAAGTGCTTCCTGTCTCGGACAGAGCTGCAGCTGC | | SRTELQLHEAFKHRGEKLFVCEEC |
| | | ATGAAGCTTTCAAGCACCGTGGTGAGAAGCTGTTTGTGTCTGAGGAG | | GHRASSRNGLQMHIKAKHRCVSPV |
| | | TGTGGGCACCGGGCCTCGAGCCGGAATGGCCTGCAGATGCACATCAA | | LSWGPVLLPAQACTGREDRVC*LL |
| | | GGCCAAGCACAGGTGCGTGTCGCCCGTTCTCTCTTGGGGCCCAGTCC | | HR*VCPWPPP*I*CLQPLWDSGT* |
| | | TGCTGCCAGCCCAGGCTTGCACCGGCAGGGAAGACAGAGTTTGCTGA | | GASSYARQRGCGQMFQAQTSLCAP |
| | | CTTTTACACAGATGAGTGTGCCCTTGGCCTCCACCCTGAATCTAGTG | | GADSELP*FPVTPAHRLSFCSRGG |
| | | CCTACAGCCTCTCTGGGATAGTGGGACCTGAGGGGCAAGCAGCTATG | | GGAPFLALPK |
| | | CCAGGCAGAGAGGCGGGGGGCAGATCTTTCAGGCTCAGACAAGCCTC | | |
| | | TGTGCCCCCGGGGCAGATTCTGAGCTTCCTTAGTTTCCTGTGACTCC | | |
| | | TGCTCATAGATTGTCCTTCTGCTCTCGGGGTCGAGGTGGTGCCCCTT | | |
| | | TCCTAGCACTGCCCAAGCC | | |
| prey107879 | 396 | CAAAGGGCGCTAATGAATGCTGTTTCGTCCTCCTGCTCCCCACACCC | 399 | QRALMNAVSSSCSPHPXTIDFXFS |
| | | CANAACTATAGACTTCANTTTTTCGGGTTTTTTTAGACGGAGTTCCG | | GFFRRSSALSPXXXXXXXXXXXXX |
| | | CTCTGTCGCCNGNNNNNNNNNNNNNNNNNNNNNNNNNNNNNNNNNNN | | XXXXXXXXXXXXXXXXXXXXXXXX |
| | | NNNNNNNNNNNNNNNNNNNNNNNNNNNNNNNNNNNNNNNNNNNNNNN | | XXXXILIYFIFKXXXXXXXXXXXX |
| | | NNNNNNNNNNNNNNNNNNNNNNNNNNNNNNNNNNNNNNCAATTTTAA | | XXXXXXXXXXXXXXXXXXXXXXXX |
| | | TTTACTTTATTTTTAAATTNNNNNNNNNNNNNNNNNNNNNNNNNNNN | | XXXXXXT |
| | | NNNNNNNNNNNNNNNNNNNNNNNNNNNNNNNNNNNNNNNNNNNNNNN | | |
| | | NNNNNNNNNNNNNNNNNNNNNNNNNNNNNNNNNNNNNNNNNNNNNNN | | |
| | | NTACCT | | |
| prey106011 | 398 | GCCTGATCTGGAAGATGTGGATGGTGTTCTCTTTGTTTCCTTTGAAT | 401 | PDLEDVIDGVLFVSFESKEALDIH |
| | | CAIIAGGAAGCTCTAGACATTCATGCAGTTGATGGGACAACAGAAGA | | AVDGTTEESSSLQASTTNDSGYRA |
| | | ATCTTCTAGTCTCCAGGCATCAACCACAAATGACTCAGGTTACAGAG | | RISQLEKELIEDLKTLRHKQVIHP |
| | | CAAGAATTTCCCAGTTGGAAAAGGAATTGATAGAAGATTTGAAGACT | | GLQEVGLKLNSVDPTMSIDLKYLG |
| | | TTGCGGCACAAGCAGGTGATACATCCTGGTCTTCAAGAAGTGGGCTT | | VQLPLAPATSFPFWNLTGTNPASP |
| | | AAAATTGAATTCAGTGGATCCAACAATGAGCATTGATCTTAAATACT | | DAGFPFVSRTGKTNDFTKIKGWRG |
| | | TGGGAGTACAGTTACCTTTGGCTCCAGCTACTAGCTTTCCTTTTTGG | | KFHSASASRNEGGNSESSLKNRSA |
| | | AACCTTACAGGAACCAACCCTGCCTCTCCTGATGCGGGATTTCCCTT | | FCSDKLDEYLENEGKLMETSMGFS |
| | | TGTTTCTAGGACAGGGAAAACCAATGATTTCACTAAGATCAAGGGAT | | SNAPTSPVVYQLPTKSTSYVRTLD |
| | | GGAGGGGAAAATTTCATAGTGCTTCTGCATCTAGGAATGAAGGTGGA | | SVLKKQSTISPSTSYSLKPHSVPP |
| | | AATTCAGAAAGTTCACTGAAAAATCGTTCTGCTTTCTGTAGTGATAA | | VSRKAKSQNRQATFSGRTKSSYKS |
| | | GCTAGATGAATACTTGGAAAATGAAGGCAAGCTGATGGAAACAAGCA | | ILPYPVSPKQKYSHVI |
| | | TGGGTTTTTCTTCTAATGCTCCCACATCTCCTGTGGTGTACCAGCTT | | |
| | | CCCACTAAGAGTACCAGTTATGTACGAACACTTGATAGTGTACTAAA | | |
| | | GAAGCAATCTACTATTTCCCCTTCTACCTCTTATTCTTTGAAACCTC | | |
| | | ATTCTGTACCCCCTGTCTCTCGAAAGGCAAAGTCTCAAAACAGACAG | | |
| | | GCAACTTTCAGTGGCCGAACTAAATCATCTTATAAATCCATTTTACC | | |
| | | ATACCCTGTTTCACCAAAGCAGAAATACTCTCATGTGATTCT | | |
| prey107880 | 400 | ATCTCACCACCATTGCCTCCCCCTCCTCCAGAGAGGCCGTGTTGGTG | 403 | ISPPLPPPPPERPCWCKAIGCGIV |
| | | CAAGGCTATAGCATGTGGTATTGTTGGCGTGGAGGATGGTGTTTGGT | | GVEDGVWLESPLRVIEGSEAPLEG |
| | | TAGAGTCACCATTGAGAGTCATAGAGGGAAGCGAAGCTCCCCTTGAA | | LFFXXXPXNLICLKTGPQXKXPXN |
| | | GGGCTTTTTTTTTTNGNTTGNCCCTNAAATTTAATTTGCCTTAAAAC | | FFFXXRXXXXXXPXXXXGXXFCXX |
| | | GGGTCCCCAGNAAAAANNCCCTNCCAATTTTTTTTTTTTNNAAAGGN | | XPSASEXXXIFXXXPXGXLXNXXX |
| | | ANNNTTNNTNCTNCCNCCCANCNTGNNGGNCAGGGGNNCANTTTTGC | | PPXPPXXFXXXXXXPR |
| | | NTNNCTNNACCCTCCGCCTCCGAANTTNAANNNATTTTCNTGNTTNN | | |
| | | NCCCCNGGGNGNCCTTNGAAATTNCNGGNCCCCCCCCNTTCCCCCCN | | |
| | | CNNAATTTTNTNNNTNCTNCNCACNCCCGCGCC | | |
| prey108056 | 402 | TCTCGCCATATTTTAACCCTTTTAATAGTGTCTCTCTTAGTGATGAC | 405 | SRHILTLLIVSLLVMTLF*VNQLP |
| | | ACTTTTTTGAGTAAATCAACTCAGAAAACTGTGTTCTTCTCTCTCTA | | AKLCSSLSNQ*ITRSNLG*FGNIN |
| | | ACCAGTGAATCACAAGGTCCAACCTAGGCTGATTTGGCAACATCAAC | | FSLLTPQYLIWGNLYLLMAIRGKN |
| | | TTTTCCCTCCTTACCCCCCAGTACCTCATTTGGGGAAATCTTTATCT | | FFXLSLXKGTPHFLXXGXXNXXXX |
| | | TTTGATGGCCATAAGGGGAAACAACTTTTTTTNCCTTTCCTTAANAA | | XGXKKGGXXPLREXXXGIGCXPP |
| | | AGGGGACCCCCCATTTTTTGGNTNTGGGANGANAAAATNTGGNGNTA | | |
| | | NGAGNGGGGNTTAAAAAAGGGGGGNTTTANCCNTTACGGGAANNGGN | | |
| | | NNGGGGGATTGGGGGGGNCCCCCCC | | |
| prey107884 | 404 | G | 407 | |
| prey107891 | 406 | NNNNNNNNNNNNNNNNNNNNNNNNNNNNNNNNNNNNNNNNNNNNNNN | 409 | XXXXXXXXXXXXXXXXXXXXXLF* |
| | | NNNNNNNNNNNNNNNNTTGTAGTTTTGACCTCCTGAGCTCANGTGAT | | PPELX*FSCFILXSSWNYXCVSPW |
| | | TCTCTTGCTTCATCCTTTTNAGTAGTTGGAACTACNGGTGTGTGTCA | | LICYFFXRGGASLCFPGXS*TAXX |
| | | CCATGGCTAATTTGTTATTTCTTTAANAGAGGTGGGGCCTCATTATG | | XXXXXXXXXXXXXXXXXLYLA*FL |
| | | TTTCCCAGGCNGGTCTTGAACTGCNNNNNNNNNNNNNNNNNNNNNNN | | NYF*RXXXXXXXXXXXXXXXXXXX |
| | | NNNNNNNNNNNNNNNNNNNNNNNNNNNNNNNNNNNCTGTACCTAGCC | | XLWPP |
| | | TAATTTTTAAATTATTTCTAGAGACNANNNNNNNNNNNNNNNNNNNN | | |
| | | NNNNNNNNNNNNNNNNNNNNNNNNNNNNNNNNNNNNACTTTGGCCTC | | |
| | | CC | | |
| prey107893 | 408 | GAATATGGCTTTTTCTAAACTTTNAATTAGAAAACATACATTAAGGG | 411 | EYGFF*TXN*KTYIKGESLPSFSA |
| | | AGAATCTCTGCCCTCCTTTTCAGCTCTGAAGATCNGCTTTTCTACTC | | LKIXFSTQTHAHTPSKCHVYGNIW |
| | | AGACACATGCACACACCCCTTCCAAGTGTCATGTTTATGGGAACATT | | EMFSRCFIFSLL*FVDI*FYLKMT |
| | | TGGGAAATGTTTTCCAGATGTTTTATTTTTTCCCTTTTATAGTTTGT | | ILIGNVRGTT*GGSS*LYTFEKYF |
| | | TGACATTTAATTTTACTTAAAGATGACAATTTTAATCGGAAATGTTA | | CFYCFLQVLXLSXILVXFSMNP*K |
| | | GAGGTACAACATAGGGAGGTTCTAGCTAGCTTTATACTTTTGAAAAA | | ENKXCXXXX |
| | | TATTTTTGTTTCTACTGCTTTTTACAAGTACTAGNCCTCTCAGNGAT | | |
| | | ACTGGTGGNGTTCAGTATGAATCCATAGAAAGAAAACAAANTTTGTT | | |
| | | GNNTAANANAG | | |
| prey107894 | 410 | TATGATTTTCTGGGTTTTGTTTACGAATTCGCATTGGCTCTCACTTG | 413 | YDFLGFV*ELALCVTCRVTLSIKL |
| | | TAGAGTCACATTATCCATTAACCTGGATAATCCCTAGTGTATGTTAT | | DNP*CMLSSEWILGLKSPMGMSVR |
| | | CCAGTGAGTGGATTTTAGGATTGAAGAGCCCAATGGGAATGAGCGTT | | CYTSRTMACLLS*LYIL*SVGHCC |
| | | CGTGGTTACACCTCTCGCACTATGGCTTGTTTGCTGTCNTGATTGTA | | *WKECFGSGLIWGTKCAW |
| | | TATCTAAAGTGTGGGACACTGCTGTTAGTGGAAAGAATGTTTTGGAA | | |
| | | GCGGTTTGATTTGGGGGACTAAATGCGCTTGGC | | |
| prey70344 | 412 | GTTCAACATAGAGAAAGGTCGGCTTGTGCAAACCCAAAGACTAAAGA | 415 | FNIEKGRLVQTQRLKIMEYYEKKE |
| | | TTATGGAATATTATGAGAACAAACAGAAACAGATTGAGCAGCAGAAG | | KQIEQQKKIQMSNLMNQARLKVLR |
| | | AAAATTCAGATGTCCAATTTGATGAATCAAGCGACACTCAAAGTCCT | | ARDDLITDLLNEAKQRLSKVVKDT |
| | | CAGAGCAAGAGATGACCTTATCACAGACCTACTAAATGAAGCAAAAC | | TRYQVLLDGLVLQGLYQLLEPRMI |
| | | AGAGACTCAGCAAGGTGGTAAAAGATACAACCAGGTACCAAGTGCTG | | VRCRKQDFPLVKAAVQKAIPMYKI |
| | | CTGGATGGACTGGTTCTCCAGGGTTTGTACCAGTTGCTGGAGCCCCG | | ATKNDVDVQIDQESYLPADIAGGV |
| | | AATGATTGTTCGTTGCAGGAAACAAGATTTCCCTCTGGTAAAGGCTG | | EIYNGDRKIKVSNTLESRLDLIAQ |
| | | CAGTGCAGAAGGCAATTCCTATGTACAAAATTGCCACCAAAAACGAT | | QMMPEVRGALFGANANRKFLD* |
| | | GTTGATGTCCAAATTGACCAGGAGTCCTACCTGCCTGAAGACATAGC | | |
| | | TGGTGGAGTTGAGATCTATAATCCAGATCGTAAAATAAAGGTTTCCA | | |
| | | ACACCCTGGAAAGCCGGCTGGATCTCATAGCCCAGCAGATCATGCCA | | |
| | | GAAGTCCGGGGAGCCTTGTTTGGTGCAAATGCCAACAGGAAGTTTTT | | |
| | | GGACTAA | | |
| prey107907 | 414 | TACTTTTTGTPATCCAAAATGGTATTTTACATTTTTTGACTATTTTG | 417 | YFL*NAKWYFTFFDYFGIL*NKPT |
| | | GCATTTTATAAAACAAGCCTACATTTTTGGAGACATTTTTAAAGAAC | | FLETFLKNPIFSKIKLIHQKLET* |
| | | CCAATCTTTTCTAAAATAAAATTAATACATCAAAAACTAGAGACATA | | MSYTSFTESYSNL*SLCTSMLDYL |
| | | AATGTCGTATACATCGTTTACTGAATCCTACAGTAATCTGTAAAGTT | | KHKKVSILRYI*YTKVQNISRQTK |
| | | TGTGTACATCAATGTTAGATTACCTAAAACATAAGAAAGTTTCTATT | | IVAATLKNNVCLANVAHACKPNTF |
| | | TTACGTTATATTTAATATACAAAAGTCCAAAATATATCCAGACAGAC | | GRP |
| | | AAAAATTGTGGCTGCCACTTTAAAAAACAATGTTTGTCTGGCCATGG | | |
| | | TGGCTCATGCCTGTAAGCCCAACACTTTTGGAAGGCCA | | |
| prey1007910 | 416 | C | 419 | |
| prey1007931 | 418 | CATATCCTGTAGACATCCTCTTAGGACATTCTCAAATTTCAAAATAA | 421 | HIL*TSS*DILKFQNKKYSSM*LI |
| | | AAAATATTCATCTATGTAGTTAATTAAAGTTAAAGTTTTTGCAGATC | | KVKVFADQLLKLLNTFT*EKVSES |
| | | AACTACTCAAACTACTAAATACATTTACCTGAGAAAAAGTCTCTGAG | | TSFLF*FV*IL*ECSGDR*LIYSG |
| | | AGCACTTCATTCCTGTTTTACTTCGTGTAAATTCTCTGAGAATGTTC | | FY*LIKVPMIFSFSAQG*WRFPFT |
| | | TGGAGATAGATAACTCATTTACAGTGCTTTCTATTAACTAATTAAAG | | F*GRIF*WGK*AF*ILLPGSAI*L |
| | | TACCCATGATTTTTTCCTTTTCTGCTCACGGATCATCGAGATTTCCT | | KIPVHTANISL |
| | | TTTACCTTCTGAGGTAGAATTTTTTAATGGGGAAAATAGGCCTTTTA | | |
| | | AATATTATTGCCAGGGTCTGCAATATAACTTAAAATTCCTGTACATA | | |
| | | CTGCAAATATTTCTTTAAA | | |
| prey5779 | 420 | CCCCAACCAAGGCTGCAACCTGGATGCCATCAAACTCTTCTGCAACA | 423 | PNQGCNLDAIKVFCNMETGETCVY |
| | | TGGAGACTGGTGAGACCTGCCTGTACCCCACTCAGCCCAGTGTGGCC | | PTQPSVAQKNWYISKNPKDKRHVW |
| | | CAGAAGAACTGGTACATCAGCAAGAACCCCAAGGACAAGAGGCATGT | | FGESMTDGFQFEYGGQCSDPADVA |
| | | CTGGTTCGGCGAGAGCATGACCCATGGATTCCAGTTCGAGTATGGCG | | IQLTFLRLMSTEASQNITYHCKNS |
| | | GCCAGGGCTCCGACCCTCCCGATGTGGCCATCCACCTGACCTTCCTG | | VAYMDQQTGNLKKALLLKGSNEIE |
| | | CGCCTGATGTCCACCGACGCCTCCCAGAACATCACCTACCACTGCAA | | IRAEGNSRFTYSVTVDGCTSHTGA |
| | | GAACAGCGTGGCCTACATGGACCAGCAGACTGGCAACCTCAAGAAGG | | WCKTVIEAYKTTKSSRLPIIDVAP |
| | | CCCTGCTCCTCAAGGGCTCCAACGAGATCGAGATCCCCGCCGAGGGC | | LDVGAPDQEFGFDVGPVCFL* |
| | | AACAGCCGCTTCACCTACAGCGTCACTGTCGATGGCTGCACGAGTCA | | |
| | | CACCGGAGCCTGGGGCAACACAGTGATTGAATACAAAACCACCAAGT | | |
| | | CCTCCCGCCTGCCCATCATCGATGTGGCCCCCTTCCACGTTGGTGCC | | |
| | | CCAGACCACGAATTCGGCTTCGACGTTGGCCCTGTCTCCTTCCTGTA | | |
| | | A | | |
| prey107934 | 422 | T | 425 | |
| prey107942 | 424 | GGGGGTGGGGACACATTGTTAAAGGTACTTTTCCTTGGATTCTTTAA | 427 | GGGDTLLKVLLLGFFKRVGVGRGL |
| | | GAGGGTGGGGGTCGGGAGAGGCTTGGCAGCATAAAGGCACGCCGCTC | | AA*RQAALG*PCS*PGCXTVEVAL |
| | | TGGGCTGACCGTGCTCCTGACCTGGATGCANAACTGTGGAAGTTCCC | | LVLLPAPSFSPCVQGRGWVFREHK |
| | | CTCCTTGTTCTGCTTCCTGCTCCCTCTTTTAGCCCGTGTGTCCAGGG | | AMAQPAAFWQFLKYSKFDRDITPA |
| | | GAGAGGATGGGTGTTCCGGGAGCACAAGGCCATGGCACAGCCAGCAG | | GARAEEQLVVCCHLTVS*FLTHCE |
| | | CCTTCTGGCAGTTCCTGAAGTACAGCAAGTTTGACAGAGATATTACC | | H*QPKYISSRQSGAQRNLCQIHSR |
| | | CCAGCTGGGGCTAGAGCAGAGGAACACTTGGTTGTGTGCTGCCATTT | | KKVLGVS*WGPTVSLKGTVNFS*D |
| | | GACAGTATCCTAATTCCTGACCCACTGTCAGCATTAACAGCCCAAAT | | LRVV*NDKPRVWLHIFLHCLCFQM |
| | | ACATATCTTCTAGACAGTCTGGTCCTCAAAGAAACCTCTGCCAGATT | | FVQCLR* |
| | | CACTCCAGGAAGAAGGTGTTGGGTGTGTCCTAGTGGGGACCCACTCT | | |
| | | GTCCCTGAAAGGGACAGTTAACTTTTCTTGAGACTTGAGAGTGGTCT | | |
| | | GAAATGACAAGCCCACACTCTGGCTGCACATTTTTTTACACTGTTTA | | |
| | | TGCTTCCAGATGTTTGTGCAGTGTCTAAGGTAAC | | |
| prey107946 | 426 | NNNNNNNNNNNNNNNNNNNNNNNNNNNNNNNNGGNAGAGTCTGGNAT | 729 | XXXXXXXXXXXXESGIQSPXLCSH |
| | | TCAGAGCCCGCANCTGTGTTCTCACCTGAGCCGTGTTCNTGGCTGGG | | LSRVXGWVXLCIXXLXVXTLXNWX |
| | | TTNTACTNTGTATTNTGNGACTNGNGGTGTNTACCTTGGNAAACTGG | | XFXFXFPLTICHVXVAXCLX*HXV |
| | | NCGNTGTTTTANTTTCNATTCCCGCTGACAATCTGTCACGTTTNTGT | | LSVIFX*AILTSXXGSXXGFNXQL |
| | | TGCTNTGTGTCTTTGNTAGCACTNGGTGTTATCAGTGATTTTTAGNT | | CSG*XC*ISCXFXPLLYPL*VSXX |
| | | CAGCCATTCTAACAAGTNTAGNGGGATCTNATTGNGGTTTTAATTNG | | IFRTXKAVXWXP |
| | | CAATTGTGTAGTGGCTAACANTGCTGAATATCATGTTNTTTTNTGCC | | |
| | | ACTCTTGTATCCTCTGTGAGTTTCTGNTCANATCTTTCGCACANAAA | | |
| | | AAGCTGTATNATGGNAACCCC | | |
| prey107952 | 428 | T | 431 | |
| prey107953 | 430 | TTTTTGATAGCTCTATGAAAGTGACAGATTTGCATCATGAGACTTAG | 433 | FLIAL*K*QICIMRLSGMNWEIEG |
| | | TGGAATGAATTGGGAAATTGAAGGGCAGCCCAGTGGCTATGGATTCT | | QPSGYGFYFLWDCLRQALFSLCSD |
| | | ATTTTCTATGGGACTGTCTACGCCAGGCTTTATTTTCTCTTTGCTCA | | NF*LESTVGL*MV*NTYSQTSFSA |
| | | GATAACTTCTGACTAGAGTCGACGGTAGGATTATAAATGGTTTAAAA | | YKFLRISV*RYVKPFARFLS*FFF |
| | | TACGTATTCTCAAACCTCATTTTCAGCATATAAATTTTTAAGAATCA | | FXLKNXKFFXEXXKXPVXXXXXXF |
| | | GTGTTTAAAGGTACGTGAAACCATTTGCTAGATTTTTGTCCTAGTTT | | LKXXXM |
| | | TTTTTTTTTNAATTAAAAAATNTTAAGTTTTTTTNAGAAANCTNAAA | | |
| | | ANGNCCAGTANTTTNTTTNCNNNCCNCNTTTTTAAAAAGNNANTANA | | |
| | | TG | | |
| prey107956 | 432 | GAGATTTTCACAACACAGATTATTTCAATCCTTGAACATAGAATATC | 435 | EIFTTQIISILAHRISFHFLMSSS |
| | | TTTCCATTTTTTGATGTCCTCTTCAATTTCTTTCATCTGTGTTTTAT | | ISFICVL*FSL*RS*LLWVIPRYL |
| | | AGTTTTCATTATAGAGATCTTAACTTCTTTGGGTAATTCCTAGGTAT | | ILFXAIIKWELLFAIXFFQTGLLG |
| | | TTAATTTTATTTGNGGCTATTATAAAATGGGAATTACTTTTTTAAAT | | GIXKRLPDFWXXXFGXPGXFXXXX |
| | | TTNCTTTTTTCAGATTGGCTTATTGGGGGGCATANAGAAAAGGCTGC | | RGXXXXXXXGGGGVXRGXP |
| | | CGGATTTTTGGNNGGNGGNTTTTGGGANNCCCGGCAANTTTNCNNAA | | |
| | | NTTAANCGGGGGTNCNAAANGNCTNTTTNTTTNGGGGGGGGGGGGGT | | |
| | | NNTTCGNGGGNCCCCA | | |
| prey107959 | 434 | N | 437 | |
| prey107963 | 436 | AAAGGATCTCTAACAGAACTNGGGTGGTTTTAGTAGAGAAGAATTAG | 739 | KGSLTELGWF* *RRIRFVSNRSY |
| | | ATTTGTTAGTAATAGAAGTTATCAAAAGTAGATTTCAGCATAATAGA | | QK*ISA**IR*IGWFLYVALANGD |
| | | TAAGGTAGATAGGATGGTTTCTATATGTAGCTTTGGCTAATGGAGAC | | FYLM*R*AD***CAAG*IVWGAYK |
| | | TTCTACTTAATGTGAAGATGAGCTGACTGATAGTGATCTGCTGCAGG | | PGTD**ELEQYSA*RFSIHIKG*R |
| | | TTAGATAGTTTGGGGTGCTTACAAACCTGGTACTGATTGATAAGAGT | | YGLIHFFP*PAYIPCSWHCPHSIG |
| | | TAGAACAATACTCAGCTTGACGTTTCTCAATTCATATTAAAGCCTAA | | NDIYSSVLRKELC |
| | | AGGTACGGGCTTATCCATTTTTTTCCCTAGCCAGCATATATCCCGTG | | |
| | | TTCTTGGCACTGTCCTCATAGTATTGGGAATGACATCTATTCTTCAG | | |
| | | TTCTAAGGAAAGAGTTGTGCC | | |
| prey67271 | 438 | ACCTCGTGTTCCTGGCGCAGAAACTGTTCAACAACAGCAGCAGCCAC | 441 | LVFLAQKLFNNSSSHLEDYSGLSV |
| | | CTGGAGGACTACAGTGGCCTGTCTGTGTCCTGGTCCCAGTTCAACAG | | SWSQFNRENLPGRNYTFWQWFDGV |
| | | GGAGAATTTACCAGGACGGAATTACACTTTCTGGCAATGGTTTGACG | | MEVLKKHLKPHWNDCAILGFVNKQ |
| | | GTGTGATGGAACTGTTAAAAAAACATCTCAAGCCTCATTGGAATGAT | | QAHDLLINKPDGTFLLRFSDSFIG |
| | | GGGGCCATTTTGGGGTTTGTAAACAAGCAACAGGCCCATGACCTACT | | GITIAWKFDSQERMFWNLNPFTT |
| | | GATTAACAAGCCAGATGGGACCTTCCTCCTGAGATTCAGTGACTCAG | | |
| | | AAATTGGCGGCATCACCATTGCTTGGAAGTTTGATTCTCAGGAAAGA | | |
| | | ATGTTTTGGAATCTGATGCCTTTTACCACCAG | | |
| prey107968 | 440 | TCTGCTTTGGGGAACACTTCTGGCACCACCAGTGGTACTTCGTATAG | 443 | SALGNTSGTTSGTSYRSYGII*GL |
| | | GTCTTATGGCATTATTTAAGGTTTAGGTTATTGCACTAAACAGGATG | | GYCTKQDAKYSRT*RDHFLLQYTI |
| | | CAAAATACTCAAGGACATAGAGAGATCACTTTTTACTGCAATACACA | | YWMNCSL*HD*GHMAF*MDTHNT* |
| | | ATTTACTGGATGAACTGTTCACTGTGACATGATTAGGGTCACATGGC | | DHCNSNKRWLQNYYSSIVGNAVNF |
| | | ATTTTAAATGGATACTCACAATACTTGAGATCATTGCAATAGCAACA | | IQLRFILYCLHLFLFI*TVNGAMY |
| | | ACAGGTGGTTACAAAATTATTACAGTAGTATAGTAGGTAATGCAGTT | | VIECV |
| | | AATTTTATCCAATTACGATTTATACTGTATTGTTTACATTTGTTTCT | | |
| | | ATTTATCTAGACTGTGAATGGTGCCATGTATGTGATCGAGTGTGTGC | | |
| | | G | | |
| prey107969 | 442 | TGGTTAGGTTGGTTTTAGTTTTGTTATTGCTTGACTGATCGTTTTTA | 445 | WLGWF*FCYCLTDRF*KMVNSLRC |
| | | AAAAATGGTTAATTCACTAAGATGTTTAGGTGCCTGTGGCAAAAAAT | | LGACGKKWQILIFKIKKKINLQPQ |
| | | GGCAAATACTAATTTTTAAGATTAAAAAGAAAATCAATTTGCAGCCT | | ELLQRK*FIYTNNYNPEK*LIIV* |
| | | CAAGAACTTTTACAAAGGAAATAATTCATTTACACAAATAATTACAA | | *VIQIRSENAEARCSLRGTYPRCP |
| | | CCCAGAAAAGTAACTTATAATAGTTTAGTGAGTTATACAAATACGCA | | HAPCSPSPIRGVASASFGQSSKRH |
| | | GTGAAAATGCAGAAGCCAGATGCTCGCTTCGGGGGACCTACCCACGC | | AHPIPSFPQRLNFCGRPMSSLPQQ |
| | | TGTCCTCACGCTCCCTGCTCTCCTTCACCCATCAGAGGAGTGGCAAG | | ILSLPPKKTTSTQTCCHVFHLQ |
| | | TGCCTCTTTCGGCCAGTCCTCCAAGCGGCATGCGCATCCCATTCCCT | | |
| | | CGTTTCCTCAAAGACTAAACTTCTGCGGCCGTCCCATGTCTTCTCTA | | |
| | | CCTCAGCAAATTCTCTCTCTCCCTCCTAAGAAAACCACTAGCACACA | | |
| | | AACCTCCTGCCATGTATTCCACCTACAG | | |
| prey107971 | 444 | NTCTATTGCACTCTTTAATTNGGNCATGGGGCTTTCAAAGAGAGGAA | 447 | XYCTL*XGHGAFKERKM*KKXXLY |
| | | AATGTGAAAAAAANAANNCTTATATTTGNTATNNNCATCTTCATCTT | | LXXXSSSLDNEKITPIAFFFFRTI |
| | | TGGACAATGAAAAGATTACCCCAATTGCATTCTTCTTTTTCCGCACA | | XGLGPQLSSERLKLSSIDLKNK*X |
| | | ATTNCTGGGTTAGGGCCACAGCTTTCATCTGAGAGATTAAAGCTTTC | | FINLQKXK*XHNLIFLLXKXRXAK |
| | | TTCTATAGACCTTAAAAATAAXIGATNTTTTATTAATTTACAAAAGN | | SFAVLSNKPIISYQLXXSLXXLLA |
| | | AAAAATAAATNCACAATTTAATCTTCTTGCTAATNAAATGNAGGTNA | | TFSGX |
| | | GCTAAGTCNTTTGCTGTTTTATCAAACAAACCTATAATTTCATACCA | | |
| | | ATTGTNTATNTCTTTAANAAANCTACTGGCTATTTTTTCAGGTTNAC | | |
| prey107976 | 446 | T | 449 | |
| prey107981 | 448 | TTCAAAGATGCCCTGCCAGTGAGGAGGAATCTAGAGAAGCAGTCTGG | 451 | FKDALPVRRNLEKQSGHNCFAVLR |
| | | CCACAACTGCTTTGCTGTGCTGCGGTCAATTCTTCCCAGTCCAAACC | | SILPSPNLPXSLALSRYNSLLKTQ |
| | | TCCCANTCTCCTTAGCACTGTCGAGGTACAACAGCCTCCTAAAGACT | | XWRMPTRLGHPTLTPIDCXAGCXN |
| | | CAATANTGGCGGATGCCCACCAGGCTCGGTCATCCCACGTTGACTCC | | FXPVVLSLLGSVTVGPTE*DHLAP |
| | | NGACTGCTNTGCTGGCTGTNAGAATTTCANGCCAGTGGTTCTTAGCT | | WH*PPFRGK*TVLSYXGSRCHVRY |
| | | TGCTGGGCTCCGTGACAGTGGGACCTACNGAGTGAGACCACTTGGCT | | KKIKLRXVXSRPXWP |
| | | CCCTGGCATTAGCCCCCTTTCCGGGGGAAGTAAACTGTTCTGTCTTA | | |
| | | CNGGGGTTCCCGGTGCCACGTAAGGTACAAAAAAATAAAACTNCGCT | | |
| | | NGGTTNGGTCCCGNCCCNAATGGCCNC | | |
| prey107987 | 450 | TTACAANACACACNCNAGATACTGAAGGAAGGTTATGCATGGATNAT | 453 | LQXRXXILKEGYAWXMIXVFKNQK |
| | | GATACNGGTATTTAAGAACCAAAAATAATGACTTGACTGAATTCTGT | | **LD*ILYSXCSNYKAK*KSG*C* |
| | | ACTCCTNATGTTCTAATTATAAAGCAAAATAAAAATCAGGGTGATGT | | K*FXISYFIKLPSLE**LISXEFE |
| | | TAAAAATGATTTANTATTAGCTATTTTATAAAACTTCCTTCCTTAGA | | LLSVLPVIFKRLTVFSFEMGVWLC |
| | | ATAATGACTCATTTCTNAAGAGTTTGAGCTTCTCTCTGTGCTTCCGG | | CPGWSTMA*SWPTATSAPPGPSNP |
| | | TGATATTCAAGAGGTTAACTGTTTTTTCCTTTGAGATGGCAGTCTGG | | PAPAXR |
| | | CTCTGTTGCCCAGGCTGGAGCACAATGGCTTGATCCTGGCCCACTGC | | |
| | | AACCTCAGCCCCGCCGGGCCCAAGCAATCCTCCCGCCCCAGCCNCCC | | |
| | | GGN | | |
| prey107993 | 452 | GCAGTGTTGTATGGAATACTCTAGCACTGTATAATTAATACAGTCNG | 455 | AVLYGIL*HCITNTVXAHTAVLVT |
| | | TGCTCACACAGCAGTCCTGGTCACTCCTTAATTTTCAGAGCTCAAAA | | P*FSELKT*XKKSSNAGMSMSTGR |
| | | CCTGAANAAAAAAAAGCTCCAATGCAGGGATGTCCATGTCCACAGGA | | QKSSYGDXCYSFARGFSXEDTKX* |
| | | AGACAAAAAAGCAGCTATGGAGATNCCTGTTATTCATTTGCCAGAGG | | FTPX*KGICGWVHLPTYXPGT*VA |
| | | CTTTTCCNGTGAGGACACCAAANAATAATTCACCCCCNAGTGAAAGG | | RTQKPPXMASISLKRALQKAMPSR |
| | | GTATCTGTGGCTGGGTTCACCTACCCACATATATNCCAGGAACTTAA | | PNDGQRVSLLANRCQSQERLAVSS |
| | | GTGGCAAGGACACAAAAACCTCCNNAGATGGCCAGTATCAGCCTGAA | | QIQVPGPGVSGKETREDERNFSSR |
| | | GAGGGCTTTGCAAAAGGCCATGCCATCAAGACCCAATGATGGACAGC | | K*DRPKGYS*VDNFSISVFSGRKG |
| | | GGGTAAGCCTCTTGGCAAACAGATGTCAGAGCCAAGAGAGGCTAGCA | | WVRSRCFSFKDLVPSHPFIYMYHT |
| | | GTGAGTAGCCAGATCCAGGTCCCAGGGCCTGCGGTGTCAGGCAAGGA | | VTECLLCARY |
| | | GACCAGAGAAGATGAGAGGAATTTCTCATCCAGGAAATAGGACAGGC | | |
| | | CTAAGGGTTATTCATAGGTGGATAATTTCTCAATATCAGTATTCTCA | | |
| | | GGAAGAAAGGGGTGGGTAAGATCAAGGTGTTTCAGCTTCAAAGACCT | | |
| | | TGTCCCTTCCCACCCATTCATTTACATGTATCATACAGTTACAGAGT | | |
| | | GCTTACTTTGTGCCAGGTACTG | | |
| prey108000 | 454 | GACACCCAGGGCAAATGTCTTCCCTTCCCTTCCNGAAGCCACAGGAA | 457 | DTQGKCLPFPSXSHRKAPWSPLYT |
| | | AGCACCCTGGAGTCCTTTGTATACCACTATNGAAATTTTTAAGGCAA | | TXEIFKASSQDLXSWNSRHLNDAX |
| | | GCTCTCAAGACTTGAANAGTTGGAATTCTCGGCATTTAAATGATGCC | | SLF*KSNISLLFSIVKQLLSRYYS |
| | | NGAAGTTTGTTTTGAAAAAGCAATATTTCATTGCTCTTTTCAATTGT | | FF*KVEThKGNIFLKPK*ITMIPV |
| | | GAAGCAACTGCTCAGCAGATATTACTCATTTTTTTAAAAAGTTGAAT | | LRDFAYFGRIKLRCXNC*ITENRC |
| | | TAAAGGGAAATATATTTTTAAAACCAAAATAGATCACAATGATCCCG | | TLRHNFKQIXX |
| | | GTTTTAAGGGATTTTGCCTATTTTGGAAGAATAAAACTNAGATGTTN | | |
| | | AAACTGTTGAATTACAGAGAACAGATGTACTCTNAGACATAATTTTA | | |
| | | AACAAATATNTGNGAT | | |
| prey108007 | 456 | TGCTCTGGGATCCCACCGAGAAGAACCATGGGTGGACCCGAACTCCC | 459 | ALGSHREEPWVDPNSPVLLEDPVL |
| | | CGGTGCTCTTGGAGGACCCAGTCCTTTGTGCCTTGGCAAAAAAGCAC | | CALAKKHKRTPALIALRYQLQRGV |
| | | AAGCGAACCCCAGCCCTGATTGCCCTGCGCTACCAGCTACAGCGTGG | | VVLAKSYNEQRIRQNVQVFEFQLT |
| | | GGTTGTGGTCCTGGCCAAGAGCTACAATGAGCAGCGCATCAGACAGA | | SEENKAIDGLNRNVRYLTLDIFAG |
| | | ACGTGCAGGTGTTTGAATTCCAGTTGACTTCAGAGGAGATGAAAGCC | | PPNYPFSDEY |
| | | ATAGATGGCCTAAACAGAAATGTGCGATATTTGACCCTTGATATTTT | | |
| | | TGCTGGCCCCCCTAATTATCCATTTTCTGATGAATATTA | | |
| prey108017 | 458 | G | 461 | |
| prey108018 | 460 | GGCTGGTTTGGCCACAGTGGCTCTCCCNACACCATTACNACNCGTCT | 463 | GWFGHSGSPXTITTRLWPFVGSYF |
| | | ATGGCCTTTTGTAGGAAGCTATTTTTGCATCTTTATCCNNAAACATT | | CIFIXKHFR*KYKYYNTLKSVHYL |
| | | TCCGTTGAAAATACAAATATTACAATACTTTGAAGAGTGTGCATTAT | | LYVYQA*DSFCLHTKLFLDI*QI* |
| | | CTTCTGTATGTTTACCAGGCATAGGATAGTTTCTGTTTACATACAAA | | KY*PFTDKTKTIH*SL*FS*WASI |
| | | GCTGTTTTTAGATATATAACAAATCTAGAAATACTGACCTTTTACTG | | NL*NTNPIFQ*IKLA*PPVATMRK |
| | | ACAAAACAAAAACAATTCATTAAAGCCTTTAATTTAGTTGATGGGCT | | *NEKGYIYQFTNSLTLYSNHMQQR |
| | | AGCATTAACCTTTGAAATACTAATCCCATTTTTCAGTGAATAAAACT | | ND |
| | | AGCATGACCTCCAGTTGCCACAATGAGAAAATGAAATGAGAAAGGGT | | |
| | | ATATATATCAATTTACGAATTCACTGACCTTGTATTCCAACCACATG | | |
| | | CAGCAGAGAAATGAC | | |
| prey108019 | 462 | G | 465 | |
| prey108020 | 464 | GGTTATTANTTAGTATTTACCCCANTCATAGTAGTTGTAAAGATTCA | 467 | VIX*YLPXS**L*RFNEXNTCKC* |
| | | ATGAANCTAATACATCTAAATGTTAAGAAACCAGTGTCCTGTCCCGT | | ETSVLSRRTWHSK*IXACYHCIAY |
| | | AGGACCTGGCACTCTAAATAAATANCTGCCTGTTATCACTGTATTGC | | KSRLETS*FSLL**LGLKFDSXVS |
| | | TTATAAATCTAGGTTAGAAACATCATAATTTTCTCTATTATAATAAT | | TKISG*SIVLSYKGSHFFLIII*F |
| | | TAGGTCTCAAATTTGACTCAGNTGTCAGCACAAAAATAAGTGGCTAA | | FSGNLDKQTA |
| | | TCTATAGTTTTAAGTTATAAAGGAAGTCATTTTTTTCTAATCATCAT | | |
| | | CTAATTTTTCAGTGGGAACCTGGATAAGCAGACGGCCC | | |
| prey108025 | 466 | GAGCTGCAACGGTCTAGGATCCTTTCTGGGGCGCCCTTCANCACCAA | 469 | ELQRSRILSGAPFXTNRCWGSDVL |
| | | CAGGTGTTGGGGCTCCGATGTGTTGGGGTTCTTATGAATAGACGACT | | GFL*IDDCEGLLRHKLWMVGCVAV |
| | | GTGAAGGATTGTTGAGACATAAACTTTGGATGGTTGGATGTGTGGCA | | HGGKQSFTYYLVFAILKDPRSLLS |
| | | GTCCATGGTGGAAAACAAAGCTTCACTTACTATTTAGTATTTGCTAT | | XLXSILHLXHTLNATEXKKXXPX* |
| | | AAAAGACCCAAGATCCCTTCTTTCTNAACTCTNAAGTATCTTACACC | | FXXNXLTFVXFKKKXPNFQXXGLG |
| | | TGTNACATACTTTGAATGCTACNGAANAAAAAAAAAANNNACCCNTT | | XXXFXXTSGXXPK |
| | | TAATTTTTNCNAAATTNCCTNACTTTTGTNCNCTTTAAAAAAAAANN | | |
| | | TCCAAATTTTCAAAANANGGGGTTGGGGNAANATTTNTTTNCACNNA | | |
| | | CNTCCGGCCNCNTCCCAAAA | | |
| prey108027 | 468 | N | 471 | |
| prey108034 | 470 | TTTGGTTTGGATATACCGTCATCCCTCGGTATCTGTGAGAGATTGGT | 473 | FGLDIPSSLGICERLVPPXIPKSV |
| | | TCCCCCAAGNATACCAAAATCTGTGGATGTGCCAGTCCCTTANATAN | | DVPVPXIXGILLGYHLXVSXCILX |
| | | ATGGTATACTACTTGCATATCACCTATNTGTATCANCTTGTATACTT | | FFFFFFXXXXWXSXPXGXEGAXFG |
| | | AANTTTTTTTTTTTTTTTTTTNNAAANNNANTTTGGNTTTCNNCCCC | | XIXPPXXGXKXXXXPXPPGXKEXP |
| | | NNCGGGNNGGGAAGGGGCNNAATTTGGGCNAATTNCCCCCCCCNTTN | | XRVXFXFXGXGGXXXXXXGGFXIP |
| | | CGGGGNTNAAGGNANTNNTGGNCCCCNNCCCCCCCGGGAANAAGGAA | | XXXEPPPXXP |
| | | TNNCCGGNCCGGGTNNNTTTTNNTTTTTTNGGNANGGGGGGGNTTNA | | |
| | | ATNNTTNGNCNGGGGGGGGTTTNNAATCCCNNNCCNNNGGGAACCCC | | |
| | | CCCCCCNGGNNCCCCC | | |
| prey108035 | 472 | G | 475 | |
| prey95798 | 474 | AGTGTTAACGTGGCTATAGATCATCTGAGCAAAGACAAACCTGAACT | 477 | SVNVAIDHLSKLDKPELEVVLTED |
| | | AGAAGTAGTCCTTACAGAGGATGCTCTTAAATCCCTAGAAAATCAGA | | ALKSLENQTYFKSFEENGKGSIIN |
| | | CATACTTCAAATCTTTTGAAGAAAATGGCAAAGGTTCCATAATTAAT | | LETRLLQLESTVSAKDLELTQCYK |
| | | TTGGAAACAAGGTTGCTACAACTTGAGAGCACTGTTAGTGCAAAGGA | | QIKDMQEQGQFETEMLQKKIVNLQ |
| | | CTTAGAACTTACCCAGTGTTATAAACAAATAAAAGACATGCAAGAAC | | KIVEEKVAAALVSQIQLEAVQEYA |
| | | AAGGCCAGTTTGAAACAGAAATGCTTCAAAAGAAGATTGTAAACCTA | | KFCQDNQTISSEPERTNIQNLNQL |
| | | CAGAAAATAGTTGAAGAAAAAGTGGCTGCTGCTCTTGTCAGTCAAAT | | REDELGSDISALTLRISELESQVV |
| | | CCAACTTGAGGCAGTTCAGCAATATGCAAAATTCTGTCAAGATAATC | | EMHTSLILEKEQVEIAEKNVLEKE |
| | | AAACAATTTCATCAGAACCTGAAAGAACAAATATTCAGAATTTAAAT | | KKLLELQKLLEGNEKKQREKEKKR |
| | | CAACTAAGAGAAGATGAGTTGGGGTCAGATATATCAGCATTAACCTT | | SPQDVEVLKTTTELFHSNEESGFF |
| | | GAGAATATCAGAATTAGAAAGCCAGGTTGTTGAAATGCATACTAGTT | | NELEALRAESVATKAELASYKEKA |
| | | TGATTTTAGAAAAAGAACAAGTAGAAATTGCAGAAAAAAATGTTTTA | | EKLQEELLVKETNMTSLQKDLSQV |
| | | GAAAAAGAAAAGAAGCTGCTAGAACTACACAAGCTATTGGAGGGCAA | | RDHLAEAKEKLSILEKEDETEVQE |
| | | TGAGAAAAAACAGAGAGAGAAAGAAAAGAAAAGAAGCCCTCAAGATG | | SKKACMFEPLPIKLSKSIASQTDG |
| | | TTGAAGTTCTCAAGACAACTACTGAGCTATTTCATAGCAATGAAGAA | | TLKISSSNQ |
| | | AGTGGATTTTTTAATGAACTCGAGGCTCTTAGAGCTGAATCAGTGGC | | |
| | | TACCAAAGCAGAACTTGCCAGTTATAAAGAAAAGGCTGAAAAACTTC | | |
| | | AAGAAGAGCTTTTGGTAAAAGAAACAAATATGACATCTCTTCAGAAA | | |
| | | GACTTAAGCCAAGTTAGGGATCACCTCGCAGAGGCAAAAGAGAAATT | | |
| | | GTCCATTTTAGAAAAAGAAGATGAGACTGAGGTACAAGAAAGCAAAA | | |
| | | AGGCCTGCATGTTTGAGCCACTTCCTATAAAACTGAGTAAGAGCATT | | |
| | | GCATCCCAGACAGATGGGACTCTGAAGATCAGTAGCAGCAATCAG | | |
| prey108040 | 476 | G | 479 | |
| prey108041 | 478 | ACTGNNNNNNNNNNNNNNNNNNNNNNNNNNNNNNNNNNNNNNNNNNN | 481 | TXXXXXXXXXXXXXXXXXXXXXXX |
| | | NNNNNNNNNNNNNNNNNNNNNNNNNNNNNNNNNNNNNNNNNNNNNNN | | XXXXXXXXXXXXXXXXXXXXXXXX |
| | | NNNNNNNNNNNNNNNNNNNNNNNNNNNNNNNNNNNNNNNNNNNNNNN | | XXXXXKXRGFGXLXXXVSXKLLAK |
| | | NNNNNNNNNNNNNNNNNCAAAANACGGGGTTTTGGCANGTTANCCNG | | VIHPPXXPKRWXXGLEPLSXAGIL |
| | | GCNGGTNTCGNTAAAACTCCTGGCTAAGGTAATCCACCCCCCCNCCC | | FFKYXFXX*NSXNX*KKNDFXXXX |
| | | NCCCAAAGCGNTGGNATNACGGGCTTGAGCCACTNTCCCNGGCCGGN | | EHXRXT |
| | | ATTTTGTTTTTTAAATACAANTTTTTNTTNTAAAATTCANTAAACAT | | |
| | | NTAAAAGAAAATGGATTTTAANANANGTNCGGAGCATTNACGGGNAA | | |
| | | CA | | |
| prey108042 | 480 | TTACTTTTTTTGAAATAAAGGAAAGGAAATTCAGACTCTTACATTGT | 483 | YFF*NKGKEIQTLTLFSVTLQF*N |
| | | TCTCTGTAACTCTTCAATTCTAAAATGTTTTGTTTTTTAAACCATGT | | VLFFKPCSDGEVDL*VWTAWTLLL |
| | | TCTGATGGGGAAGTTGATTTGTAAGTGTGGACAGCTTGGACATTGCT | | SCG*R*CLHS*RANNSI*HIVYTY |
| | | GCTGAGCTGTGGTTAGAGATGATGCCTCCATTCCTAGAGGGCTAATA | | IFMSKKNKNLSNRAL*YCQREKQI |
| | | ACAGCATTTAGCATATTGTTTACACATATATTTTTATGTCAAAAAAA | | LKIHGNVT*FRVGIFLKIHQYLTF |
| | | AACAAAAACCTTTCAAACAGAGCATTGTGATATTGTCAAAGAGAAAA | | FKKITLKQHTVRKNSIDIP |
| | | ACAAATCCTGAAGATACATGGAAATGTAACCTAGTTTAGGGTGGGTA | | |
| | | TTTTTCTGAAGATACATCAATACCTGACCTTTTTTAAAAAAATAATT | | |
| | | TTAAAACAGCATACTGTGAGGAAGAACAGTATTGACATACCCAC | | |
| prey56754 | 482 | A | 485 | |
| prey108048 | 484 | TGGAAGGTTTATCCNTCTATCATTTGATGGACGTTTGGGTTGTTTCT | 487 | WKVYPSII*WTFGLFLHFYCYE*C |
| | | ACATTTTTATTGTTATGAATAATGCTGCAAAAAACATGTGTTTTCTG | | CKKHVFSVFLSIYIHLGVKLLSHM |
| | | TTTTCTTGAGTATATATATACACCTAGGAGTAAAATTACTGAGTCAT | | VILCVAFEXLSSFCPQGYTVLHSH |
| | | ATGGTAATTCTGTGTGTAGCTTTTGAGNAACTGTCAAGTTTTTGCCC | | QQCTRVPVSPHLTNTCYNHASGV* |
| | | ACAGGGCTATACCGTTTTACATTCCCACCAGCAATGTACGAGGGTTC | | KDYCFLLKYISERYISRIDSILPQ |
| | | CAGTTTCTCCGCATCTCACCAACACTTGTTATAACCATGCTAGTGGG | | QLVGKSVDFNIGLYVLGGWTQTSS |
| | | GTGTGAAAGGACTATTGCTTTTTGCTAAAGTATATCAGTGAAAGATA | | GGDESFV*TKPV*CVSFIR*YSKT |
| | | CATTTCACGTATTGATAGCATATTGCCACAGCAATTGGTGGGGAAAT | | IQ*LRCANWPG |
| | | CAGTGGATTTCAATATCGGTTTATATGTTCTAGGAGGATGGACTCAG | | |
| | | ACAAGTTCTGGAGGAGATGAAAGCTTTGTATGAACAAAACCAGTCTG | | |
| | | ATGTGTAAGTTTCATAAGATGATATTCTAAAACAATTCAATAATTAA | | |
| | | GATGTTGAAATTGGCCGGGCG | | |
| prey108049 | 486 | GCANACNAGGCCTGTTTTACAACCCCAACCAGGAAGTCTGAATTCCA | 489 | AXXACFTTPTRKSEFQYLGWIXSS |
| | | GTATCTGGGATGGATCTNAAGTTCTGAGGCGAATCCGAGGACCACTG | | EANPRTTACSGNAYGVCIXICFAS |
| | | CTTGCTCAGGAAATGCCTACGGTGTCTGTATTANAATATGCTTTGCC | | XAPPGSXK*SGPF*TSITKAVNFT |
| | | TCANAGGCCCCCCCAGGGTCCANAAAATGATCGGGACCTTTCTGAAC | | YHWQSSC*SCPHRHPT*ADGCTQL |
| | | GAGCATTACCAAGGCGGTCAACTTCACCTATCATTGGCAGTCCTCCT | | HPTDSVSEACPCSAPNATSLSCSL |
| | | GTTAGAGCTGTCCCCATACGCACCCCACCTAAGCAGATGGCTGTACC | | W*EDVSKPALQCPELFPPGSPFSS |
| | | CAGCTTCACCCAACAGATTCTGTGTCCGAAGCCTGTCCATGTTCGGC | | *CSSCSQPPPESTASWRSTATAWT |
| | | CCCCAATGCCACCTCGTTATCCTGCTCCCTATGGTGAGAGGATGTCT | | DVS |
| | | CCAAACCAGCTCTGCAGTGTCCCGAACTCTTCCCTCCTGGGTCACCC | | |
| | | TTTTCCTCCTAGTGTTCCTCCTGTTCTCAGCCCCCTCCAGAGAGCAC | | |
| | | AGCTTCTTGGAGGAGCACAGCTACAGCCTGGACGGATGTCTCCC | | |
| prey108051 | 488 | TGACANNATCNCTNATAAAAAGTNCCNTNAATACTNTTNTTNGATTG | 491 | *XXXX*KVPXILXXDWXRITXAXX |
| | | GNNAAGGATAACANGGGCGGNGANCTTTTGGACTTNNTTTTTTTNTT | | FWTXFFXYXPTPXLXSXFXQXXTP |
| | | ATTGNCCNACCCCTTNCTTGNNNTCTCNNTTCCNACAANGNTTNACG | | KTXGEXLKXLXAWRSRX |
| | | CCNAAAACTTNAGGGGAANTTTTAAAATTNCTTNAGGCNTGGCGTTC | | |
| | | CCGGNGNGG | | |
| prey108052 | 490 | CGTGGTGCCAAAAACGTTGGGGAGCTCTGCTTTAAATCATTTCTAGG | 493 | RGAKNVGELCFKSFLGYF*YLIQC |
| | | TTACTTCTAATACCTAATACAATGTAAATGCTATATACACACTTGTT | | KCYIHSCYTIF*FTLFFIFVLLFF |
| | | ATACTATATTTTAATTTACATTATTTTTTATTTTTGTGTTGTTATTT | | IFFSLNIFDPCLIKSVDVETTGWR |
| | | TTCATCTTTTTTTCTTTGAATATTTTTGATCCATGTTTGATTAAATC | | ASCT |
| | | CGTGGATGTGGAAACCACAGGTTGGAGGGCCAGCTGCACAC | | |
| prey3794 | 492 | A | 495 | |
| prey108059 | 494 | TTTGATTTCACTTGTTCTGCTTCTTCTCTTGCTGTAGTTGCCACGTG | 497 | FDFTCSASSLAVVATCPDISQNFV |
| | | CCCTGACATTTCGCAGAACTTTGTTGCCATCCATCCAGATTTGGCCA | | AIHPDLAISPLIPPVAPHCKCDTL |
| | | TTTCTCCTTTAATTCCACCTGTTGCCCCTCATTGTAAGTGTGACACA | | IFFGLF*GIN*TCLKFRRV*ESIC |
| | | CTTATCTTCTTTGGTTTATTCTAAGGCATTAACTAAACATGTCTGAA | | VCVYKKXXXXXXXXXXXXXXXXXX |
| | | ATTCCGACGAGTCTAAGAATCAATATGTGTGTGTGTATACAAAAAGT | | XXXXXXXXXXXXXXXXXXXXXXXX |
| | | NNNNNNNNNNNNNNNNNNNNNNNNNNNNNNNNNNNNNNNNNNNNNNN | | XXXXXXXXXX |
| | | NNNNNNNNNNNNNNNNNNNNNNNNNNNNNNNNNNNNNNNNNNNNNNN | | |
| | | NNNNNNNNNNNNNNNNNNNNNNNNNNNNNNNNNNNNNNNNNNNNNNN | | |
| | | NNNNNNNNNNNNNNNNT | | |
| prey108062 | 496 | GNAACTCAGGATGGATTTCANTGGTCCAATTATGGCTCATTGNAGCC | 499 | XTQDGFXWSNYGSLXPQTPRLKQS |
| | | TCAAACTCCCAGGCTCAAGCAGTCCTACNATGTCNGCCTTCCCAGTA | | YXVXLPSXWDHRLGLTLSSRXECX |
| | | NCTGGGACCACAGACTGGGTCTCACTCTNTCGTCCAGGCNGGAGTGC | | GTILAXCXVGFXGSRDSPTPNSXV |
| | | ANTGGCACAATCTTGGCTCANTGTANTGTAGGCTTCCNAGGCTCAAG | | AXTTGVHHHHXWXMFXFLXXTESX |
| | | GGATTCTCCCACCCCAAACTCCCNAGTAGCTNGAACTACGGGTGTGC | | YFALIGLQLRA*SDPPAXASENAL |
| | | ACCACCACCACANCTGGNTAATGTTTNTTTTTTTGGNANACACAGAG | | DYXALNH*XPGXMX |
| | | TCTNACTATTTTGCGCTNATTGGTCTCCAACTNCGCGCCTGAAGCGA | | |
| | | TCCTCCTGCCTNGCCTTCCGAAAATGCTTTGGATTACNGGGCNTTGA | | |
| | | ACCATTAANGCCCTGGCNTNATGNTNN | | |
| prey108066 | 498 | CTTCTCTTAATTATTTGAATAAGGTAGTTTGGAATAAAGAAAGAAAA | 501 | LLLII*IR*FGIKKEKITLHTEL* |
| | | GATCACTCTACATACAGAACTGTAAACTTAATTTGTGATCCTATATA | | T*FVILYMRQYKNTDKF*KDSKQY |
| | | TGAGACAGTATAAAAATACAGATAAGTTTTAGAAAGACTCAAAACAA | | VND*CLHY*GKLGMLGQEGKVLVN |
| | | TATGTAAATGACTGATGTTTGCATTATTAAGGAAAACTTGGGATGTT | | PLWSNIMKVNYNSIYLSLMPQSEI |
| | | GGGTCAAGAGGGGAAAGTGTTAGTCAATCCACTTTGGAGCAATATCA | | EYSLGGHGCAPIQYTFFG*ICFQI |
| | | TGAAGGTCAATTATAATTCCATATACCTTTCTTTGATGCCACAGTCA | | ISWNL*SIFDSNFD |
| | | GAGATAGAATACAGTTTGGGTGGCCATGGATGTGCCCCAATACAGTA | | |
| | | CACATTTTTTGGTTAAATTTGTTTTCAGATCATTTCATGGAATCTTT | | |
| | | GAAGTATCTTTGACTCTAACTTTGAC | | |
| prey108070 | 500502 | CTTGTGGCATTTGTATTATAAAAACTTGGTGTGGGGGAGGAGCACAA | 503 | LVAFVL*KLGVGEEHKTPAH*TSA |
| | | AACTCCAGCCCACTGAACCTCTGCCAATTAAGATGGTGTTGGGTTAG | | N*DGVGLGYIWLLSWENHFYRDGL |
| | | GTTACATCTGGTTACTGTCCTGGGAAAATCATTTTTATAGAGATGGC | | PSGFKIY*SF*VNYVC*LNLQINL |
| | | CTTCCAAGTGGTTTTAAAATTTACTGAAGTTTTTAGGTCAATTATGT | | FIQLSVQNLN*MY*VFTCPII*VL |
| | | ATGTTGACTAAATTTACAAATAAACTTGTTTATCCAACTAAGTGTCC | | VY*CFELRSFQVLFGG*RNLLFIK |
| | | AAAACCTAAATTGAATGTACTAAGTTTTCACATGTCCCATTATCTAG | | IL*KACEQLSA*LRCHNSAVFAVW |
| | | GTCCTTGTATACTAATGTTTTGAACTTAGATCATTTCAGGTGTTGTT | | AKTKYIKL |
| | | TGGTGGATAAAGGAACCTTTTATTTATAAAGATACTGTAGAAAGCAT | | |
| | | GTGAACAGCTCTCTGCTTGATTAAGATGCCATAATAGTGCTGTATTT | | |
| | | GCAGTGTGGGCTAAGACAAAGTATATTAAGCTT | | |
| prey108072 | 504 | A | 505 | |
| prey108073 | 506 | GGTGGTGTGTCCTTTTTAGGTTGGCTATAATGTTTGGTTCTACAGGC | 507 | GGVSFLGWV*CLVLQALLT*KNGW |
| | | TCTACTAACATAAAAAAATGGATGGCTGGAGTTGCTTGGCTACACTG | | LELLGYTVCCPKWIXWXMTF*NAW |
| | | TTTGTTGCCCAAAATGGATTCNGTGGTNTATGACTTTTTAAAATGCA | | CTTFLKKDTGCLKDQLIVVKLHXH |
| | | TGGTGTACAACATTCCTAAAAAAAGATACTGGCTGTTTAAAGGACCA | | LLCLNYVGGKL*MLICPWTG*TLS |
| | | ATTGATAGTGGTAAAACTACATTANCATCTGCTTTGCTTGAATTATG | | *E*LLTSF**FLRMRALEGPXICL |
| | | TGCGGGGAAAGCTTTAAATGTTAATTTGCCCTTGGACAGGCTGAACT | | QV |
| | | TTGAGCTAGGAGTAGCTATTCACCAGTTTTTAGTAGTTTTTGAGGAT | | |
| | | GTAAAGGGCACTGGAGGGGAGTCCAGANATTTGCCTTCAGGTCA | | |
| prey108074 | 508 | G | 509 | |
| prey108075 | 510 | N | 511 | |
| prey108076 | 512 | TTCTCTGCCCCTTTCTTTCCACCCTGTACATACTGCCTTGTTTCAAA | 513 | FSAPFFPPCTYCLVSNPHHLPAYI |
| | | CCCTCATCATCTTCCTGCATATATCCAACAGATNCATAAACTCAGTG | | QQXHKLSGFQVICYGSFW*KVPSS |
| | | GTTTTCAAGTCATCTGTTATGGGAGTTTTTGGTAAAAGGTGCCCTCT | | HCSDGSPVLIFNRISVCGFHWSPY |
| | | TCCCACTCCAGTGATGGTAGCCCTGTTTTAATCTTTAATAGAATAAG | | HSSLLLPSIWLPDILKTKLIIWLF |
| | | CGTTTGTGCATTTCACTGGTCTCCTTACCACTCTTCACTTCTCCTGC | | SC*NLPWSSSCRVVFVNSKRLQGL |
| | | CTTCCATCTGGCTNCCTGACATTTTGAAAACAAAGCTAATCATATGG | | ER*VKWMK*TGCKSSGRREKCCGK |
| | | CTTTTTTCCTGCTGAAACCTTCCTTGGTCCTCATCATGTAGGGTGGT | | LSHLKGTPASCIHVGMWAHFVIVP |
| | | ATTTGTAAACTCAAAACGCCTGCAGGGACTAGAAAGGTGAGTTAAAT | | SFLKRSQKFRLL*EMF*LNIDFSR |
| | | GGATGAAGTGAACTGGATGCAAGAGCAGTGGGAGAAGGGAGAAGTGC | | VFKMQTN |
| | | TGTGGCAAACTGTCCCATCTAAAAGGGACGCCTCCCTCTTGTATCCA | | |
| | | CGTGGGAATGTGGGCCCACTTTGTCATAGTGCCCAGTTTTTTAAAAA | | |
| | | GAAGCCAGAAATTCAGATTGTTATGAGAAATGTTCTGATTGAATATT | | |
| | | GACTTTAGTCGAGTTTTTAAAATGCAGACCAAC | | |
| prey64719 | 514 | GTCGTCCAACCCATCATCCCGGGACTCTTCTCCAAGCAGGGACTTCT | 515 | SSNPSSRDSSPSRDFLPALGSMRP |
| | | TGCCAGCCCTTGGCAGCATGAGGCCTCCCATCATCATCCACCGAGCT | | PIIIHRAGKKYGFTLRAIRVYMGD |
| | | GGCAAGAAGTATGGCTTCACCCTGCGGGCCATTCGCGTCTACATGGG | | SDVYTVHHMVWHVEDGGPASEAGL |
| | | TGACTCCGATGTCTACACCGTGCACCATATGGTGTGGCACGTGGAGG | | RQGDLITHVNGEPVHGLVHTEVVE |
| | | ATGGAGGTCCGGCCAGTGAGGCAGGGCTTCGTCAAGGTGACCTCATC | | ThILKSGNKVAISTTPLENTSTKV |
| | | ACCCATGTCAATGGGGAACCTGTGCATGGCCTGGTGCACACGGAGGT | | GPARKGSYKAKMARRSKRSRGKDG |
| | | GGTGGAGCTGATCCTGAAGAGTGGAAACAAGGTGGCCATTTCAACAA | | QESRKRSSLFRKITKQASLLHTSR |
| | | CTCCCCTGGAGAACACATCCATTAAAGTGGGGCCAGCTCGGAAGGGC | | SLSSLNRSLSSGESGPGSPTHSHS |
| | | AGCTACAAGGCCAAGATGGCCCGAAGGAGCAAGAGGAGCCGCGGCAA | | LSPRSPTQGYRVTPDAVHSVGGNS |
| | | GGATGGGCAAGAAAGCAGAAAAAGGAGCTCCCTGTTCCGCAACATCA | | SQSSSP |
| | | CCAAGCAAGCATCCCTGCTCCACACCAGCCGCAGCCTTTCTTCCCTT | | |
| | | AACCGCTCCTTGTCATCAGGGGAGAGTGGGCCAGGCTCTCCCACACA | | |
| | | CAGCCACAGCCTTTCCCCCCGATCTCCCACTCAAGGCTACCGGGTGA | | |
| | | CCCCCGATGCTGTGCATTCAGTGGGAGGGAATTCATCACAGAGCAGC | | |
| | | TCCCCCAG | | |
| prey108082 | 516 | GCCTCCACCTTTACAAAAACGAGCTTAAAGATATTAGGAATATTTTA | 517 | ASTFTKTSLKILGIF*L*NTQVKT |
| | | ATTATAAAATACTCAAGTAAAAACACCACCTTCACCATACAGTTCTT | | PPSPYSSLHYRIGCS*LEFLLPSF |
| | | TACATTACCGAATTGGCTGTAGTTGATTAGAGTTTCTGCTACCAAGC | | LSIMS*WVKSTSNFSHA**LGHSI |
| | | TTTCTTTCTATCATGTCATAATGGGTCAAAAGTACCAGCAATTTCTC | | FSRVNL*NNNKSGLWYLITFTNLS |
| | | ACATGCATAGTGATTGGGCCACTCCATTTTTTCAAGAGTAAATCTTT | | LFKRNS*ITSWNGNPSQQQAFFLI |
| | | GAAACAATAACAAATCAGGTCTTTGGTACCTGATAACCTTCACCAAT | | LFSTSXSCLSCSVRCHSQGQQYL* |
| | | TTATCCTTGTTTAAAAGGAATTCTTGAATAACCTCATGGAATGGGAA | | SCGSQYLSLLHNLHPLRS*SLGEP |
| | | TCCCTCACAACAGCAAGCTTTCTTCTTAATATTGTTCTCTACTTCAN | | LWRPKKSIIFFAFYI |
| | | TGAGTTGCCTATCATGTTCTGTACGGTGCCACTCACAAGGACAGCAG | | |
| | | TATTTATACTCATGTGGCTCACAATATTTATCACTTTTACATAATCT | | |
| | | GCATCCATTACGTTCATGATCCTTAGGTGAACCTTTATGGAGACCCA | | |
| | | AAAAAAGTATAATATTTTTTGCCTTTTATATTAC | | |
| prey108083 | 518 | TCCTGGATTCTTTGATAATGGTATATTGGAAACTAAGGTATTAATAG | 519 | CWIL**WYIGN*GINSFIISXLLW |
| | | TTTTATAATCTCTCANCTGCTCTGGAAATAATGTGCTAATGAGGTCT | | K*CANEVSPSEVGKLhRXLETKRS |
| | | CCCCTTCCGAAGTTGGTAAACTGCACAGAANCCTGGAAACTAAACGA | | DY*ILLF*RISNRFHYISFNYRLN |
| | | AGTGACTACTCAATTTTACTTTTCTGAAGGATTTCCAATAGATTTCA | | LL*TKLQGYIYSTYVILYIFLSTM |
| | | TTATATTTCCTTTAACTACACGTTAAATCTCTTATAAACCAAACTGC | | KEPFPHQ*HDVINPARHMASDVHQ |
| | | AGGGTTACATATACAGTACATATGTTATCTTGTATATTTTTCTTAGT | | AAQNRQ*M*NTRANFQSV*SLT*V |
| | | ACTATGAAGGACCCTTTCCCTCATCAATAACATGATGTTATAAACCC | | KYTNSRKMASFITQAVI*KQS |
| | | TGCCAGACATATGGCNTCAGACGTACATCAGGCTGCACAGAATAGGC | | |
| | | AATGAATGTAGAACACTAGAGCTAATTTTCAGTCTCTATGAAGTTTA | | |
| | | ACTTGAGTCAAATACACCAATTCAAGGAAAATGGCTTCTTTTATCAT | | |
| | | CCAAGCTGTTATCTAAAAACAATCT | | |
| prey108088 | 520 | GCTGAGGTTCAGGGCGGACTCTCCGGGAGACTGTCCTGGAAAGTTCG | 521 | *GSGRTLRETVLESSPILTLLNES |
| | | CCCATCCTCACCCTGCTCAACGAGACCTTCATCAGCACCTGGTCCCT | | FISTWSLVKELEELQNNQENSSHQ |
| | | GGTGAAGGAGCTGGAGGAACTGCAGAACAACCAGGAGAACTCGTCCC | | KLAGLHLEKYSFPVEMMICLPNGT |
| | | ACCAGAAGCTGGCTGGCCTGCACCTGGAGAAGTACAGCTTCCCCGTG | | VVHHINANYFLDITSVKPEEIESN |
| | | CAGATGATGATCTGCCTGCCCAATGGCACCGTGGTCCATCACATCAA | | LFSFSSTFEDPSTATYMQFLKEGL |
| | | TGCCAACTACTTCTTGGACATCACCTCCGTGAAGCCCGAGGAAATCG | | RRGLPLLQP* |
| | | AGAGCAATCTCTTCAGCTTCTCATCCACCTTTGAAGACCCCTCCACG | | |
| | | GCCACCTACATGCAGTTCCTGAAGGAGGGACTCCGGCGTGGCCTCCC | | |
| | | CCTCCTCCACCCCTAG | | |
| prey108093 | 522 | CAAGGGCGCTAATGAATGCTGTTTCGTCCTCCTGCTCCCCACACCCC | 523 | QGR**MLFRPPAPHTPXLXTSXFR |
| | | ANAACTANATACTTCANTTTTTCGGGTTTTTTTAGACGGATTTCCCC | | VFLDGFPLCRRAXXXXXXXXXXXX |
| | | TCTGTCGCCGGGCAAANNNNNNNNNNNNNNNNNNNNNNNNNNNNNNN | | XXXXXXXXXXXXXXXXXXXXXXXX |
| | | NNNNNNNNNNNNNNNNNNNNNNNNNNNNNNNNNNNNNNNNNNNNNNN | | XXXQF*FTLFLNXXXXXXXXXXXX |
| | | NNNNNNNNNNNNNNNNNNNNNNNNNNNNNNNNNNNNNCAATTTTAAT | | XXXXXXXXXXXXXXXXXXXXXXXX |
| | | TTACTTTATTTTTAAATTNNNNNNNNNNNNNNNNNNNNNNNNNNNNN | | QV*AT |
| | | NNNNNNNNNNNNNNNNNNNNNNNNNNNNNNNNNNNNNNNNNNNNNNN | | |
| | | NNNNNNNNNNNNNNNNNNNNNNNNNNNNNNNCAGCTNTGAGCCACCG | | |
| | | T | | |
| prey108097 | 524 | GTTAGTAATGTCATTTATCTNAAAGTCTACTTTGGCTAAAATTAATA | 525 | VSNVIYLKVYFG*N**FMLAFFLL |
| | | ATTTATGTTAGCTTTCTTTTTGTTAGTATTTGCATGTAATATCTTTT | | VFACNIFFHYFTFNFQYFCSLLFX |
| | | TCCATTATTTTACCTTCAATTTTCAATATTTCTGTAGCCTTTTGTTT | | IHSWLKQYVIKFFFSXSFIXGFFF |
| | | CANATACATTCCTGGTTAAAACAGTATCTAATAAAATTTTTTTTTTC | | FXXXXXXXXPXPGGXGGXXGXXXP |
| | | CCANAGTTTTATTAANGGCTTTTTTTTTTTTNNAAANNANNTTTTNN | | PPPGVXXXXPPPFPGXXKXR |
| | | NTTTNGNCCCCCNCCCGGGGGGGNANGGGGGGGNTTNNGGCNAANTN | | |
| | | NNCCCCCCCCCCCCGGGGGTNANGGNANTTNNCCCCCCCCCGTTCCC | | |
| | | GGGNNNGNTNAAATANCGG | | |
| prey108102 | 526 | A | 527 | |
| prey108100 | 528 | A | 529 | |
| prey108105 | 530 | TGCCTAGACAAACTGTTTTGGAAAAAGATGCTTACATCAACCATAAT | 531 | CLDKLFWKKMLTSTIIMHFMVFFS |
| | | TATGCATTTTATGGTATTTTTTAGCCCTCTAAAAAGGGAATCCTCCC | | PLKRESSLNLXI*KSKFVLFLHSH |
| | | TAAACCTCTNAATCTAAAAATCTAAGTTTGTTTTGTTTCTACATTCC | | FXIXSWKQIKH*GSSIAHFSSHNE |
| | | CATTTCCNAATATNATCATGGAAGCAAATTAAGCACTGAGGTTCGTC | | HIYICRNSSFNTLLGEFVFPPSLT |
| | | TATAGCTCATTTCTCTTCTCACAATGAACATATCTACATCTGCAGAA | | DCL*LWLSSLRTDKIY*S*NFSSK |
| | | ATTCATCTTTTAATACTCTTCTAGGGGAATTTGTGTTCCCACCTTCC | | L*QSQQLAPSHLHRLEVQQYRFSL |
| | | CTAACTGATTGTCTTTAGCTATGGCTATCCTCTCTCAGGACTGATAA | | MILL*LTPIPVINTWLCFKGCIFF |
| | | GATTTACTGAAGCTGAAACTTCTCATCTAAACTCTGACAATCCCAGC | | LAIDNHILKNSVLSL |
| | | AACTGGCCCCCAGCCACCTGCACCGGCTAGAGGTTCAACAATACAGA | | |
| | | TTCTCCTTAATGATCCTACTCTAGTTGACTCCTATCCCAGTAATTAA | | |
| | | TACATGGCTGTGCTTCAAAGGCTGCATTTTTTTTCTCGCTATAGATA | | |
| | | ATCATATTCTTAAAAATAGTGTTTTATCCTTAAA | | |
| prey108107 | 532 | TGTACTTTACCTTTAGCTGTGAATTCTTCCTATTTGACACTCCTTAC | 533 | CTLPLAVNSSYLTLLTTPSPDPPX |
| | | CACCCCNTCCCCNGACCCTCCNTNATGTAAAAACTTCAAAGAGATCT | | CKNFKEILLGIF*ILETRAGIDLC |
| | | TGTTAGGTATCTTTTGAATCTTGGAGACACGGGCAGGAATTGACTTA | | QKRKPLSERLAEVNSISTAEGCT* |
| | | TGCCAAAAGAGAAAGCCTTTGAGTGAGAGGCTGGCTGAAGTGAACAG | | EVSTFWLMPGQSCIFLGTK*NPQ* |
| | | TATCAGCACAGCAGAGGGCTGCACTTAGGAAGTAAGCACTTTTTGGC | | ECESELFYKEF*PRGLPGMDEVSL |
| | | TTATGCCAGGCCAAAGTTGTATTTTTCTGGGGACCAAATAAAATCCA | | TKRG*RFHWMEK*REIVNSCLKER |
| | | CAGTAGGAATGTGAGTCTGAGTTGTTTTATAAGGAATTCTAACCCAG | | HYLSEGTPVG |
| | | AGGGCTTCCTGGAATGGATGAGGTGTCCTTGACAAAAAGAGGTTGAA | | |
| | | GGTTTCACTGGATGGAGAAGTAGAGGGAGATTGTAAACAGCTGCTTA | | |
| | | AAGGAGAGGCACTACCTGAGTGAAGGAATTCCAGTTGGAC | | |
| prey108108 | 534 | TTCTTTGGATATTTGTGATAATTGTGGTGGCTCAGGCTAAATCTAGA | 535 | FFGYL**LWWLRLNLDF*MLSISL |
| | | TTTTTAAATGCTTTCGATTTCCTTAGCGCCTATCACGGACTGGCCCT | | APITDWPSVFLTQPRDLILTTSW* |
| | | CTGTTTTCCTTACACAACCCCGCGATCTGATTCTCACTACATCGTGG | | DRYYHPCWADE*M*LSRN*ILIAC |
| | | TGAGACAGGTATTACCATCCCTGTTGGGCAGATGAATGAATGTAGCT | | LEINSEYSKDFYPGPPGFQVHVLL |
| | | ATCAAGAAATTAAATATTAATAGCATGTCTGGAGATCAATAGCGAGT | | PYKCGSASELEDTESWPEGA*SER |
| | | ATAGTAAAGACTTTTACCCAGGTCCACCTGGTTTCCAAGTTCATGTC | | DSLVLK*ELFFYF*HLIIRQFP*S |
| | | CTTCTTCCCTATAAGTGTGGTAGTGCGTCAGAACTGGAGGACACAGA | | LRYSTKKKFXXXXXXXXXXXXXXX |
| | | ATCCTGGCCTGAAGGTGCATAGTCAGAAAGGGATTCCTTAGTCTTAA | | XXXXXXXXXXXXXXXXXXXXXXXX |
| | | AATAAGAGTTGTTTTTTTATTTTTAGCATCTTATAATCCGGCAATTT | | XXXXXXXXXXXXXXXPAA |
| | | CCTTGAACCCTTAGATACAGCACAAAAAAGAAATTCAANNNNNNNNN | | |
| | | NNNNNNNNNNNNNNNNNNNNNNNNNNNNNNNNNNNNNNNNNNNNNNN | | |
| | | NNNNNNNNNNNNNNNNNNNNNNNNNNNNNNNNNNNNNNNNNNNNNNN | | |
| | | NNNNNNNNNNNNNNNNNNNNNNNNNNNNNNNNNNNNNNNNNNNNNNN | | |
| | | NNNNNNNNNCCCAGCCGCC | | |
| prey51316 | 536 | CAGGGTGCCCCTGGAACTCCTGGCCCTGTGGGTGCTCCAGGAGATGC | 537 | QGAPGTPGPVGAPGDAGQRGDPGS |
| | | AGGACAAAGAGGAGATCCGGGTTCTCGGGGTCCTATAGGACACCTGG | | RGPIGHLGRAGKRGLPGPQGPRGD |
| | | GTCGAGCTGGAAAACGTGGATTACCTGGACCCCAAGGACCTCGTGGT | | KGDHGDRGDRGQKGHRGFTGLQGL |
| | | GACAAAGGTGATCATGGAGACCGAGGCGACAGAGGTCAGAAGGGCCA | | PGPPGPNGEQGSAGIPGPFGPRGP |
| | | CAGAGGCTTTACTGGTCTTCAGGGTCTTCCTGGCCCTCCTGGTCCAA | | PGPVGPSGKEGNPGPLGPLGPPGV |
| | | ATGGTGAACAAGGAAGTGCTGGAATCCCTGGACCATTTGGCCCAAGA | | RG |
| | | GGTCCTCCAGGCCCAGTTGGTCCTTCAGGTAAAGAAGGAAACCCTGG | | |
| | | GCCACTTGGGCCATTGGGACCTCCAGGTGTACGAGGCAG | | |
| prey108109 | 538 | GGGGAGTGTCTGTATATCCTTTAAAACTTTGTTTACAAAGTTTACAC | 539 | GECLYIL*NFVYKVYTHTVCLQKQ |
| | | ACATACTGTATGTCTACAGAAACAATAGTTTTCCTTTGCATTTTTAA | | *FSFAFLN*KQYCSAACFSNNI** |
| | | ACTAGAAACAATATTGTTCAGCAGCCTGCTTTTCTAACAATATATAA | | YITFIETAGYFLIVEAEYSPDQLQ |
| | | TAATACATAACATTTATTGAGACAGCTGGTTATTTTCTCATAGTGGA | | GQRGVKLQQYISSYFLLCTHMQAH |
| | | GGCAGAGTATTCCCCTGACCAATTACAAGGGCAGAGGGGTGTGAAAC | | FLREDTMK*T*LVYLVVWFLNVDI |
| | | TACAGCAATATATATCCTCGTATTTTCTCCTCTGTATACATATGCAG | | IKLSFKIKRTVNX*VKFLTCQXXK |
| | | GCACATTTCCTGAGAGAAGATACTATGAAGTGAACTTAACTAGTGTA | | ANXSSFXPSXXTSQXRTNIIKYWR |
| | | TCTAGTAGTTTGGTTTTTAAATGTTGACATTATCAAATTGTCCTTTA | | *QWYTVXATIPQTGGGKKKIIQST |
| | | AAATAAAAAGAACAGTTAACTNTTAAGTGAAATTTTTAACTTGTCAA | | IK*ILTK |
| | | TNTANAAAAGCCAACCANAGCTCTTTTTTNCCTTCCTNCCNAACTTC | | |
| | | CCAGNCCAGAACAAATATAATTAAATACTGGCGATAACAGTGGTATA | | |
| | | CAGTANAAGCAACCATCCCTCAGACCGGAGGGGGGAAAAAAAAAATC | | |
| | | ATCCAATCAACTATTAAGTAGATCTTAACAAAAA | | |
| prey108110 | 540 | GCCACCACTGCCGGGGACTGGAGTTTGTTGGATGTGAGGGGAATGCC | 541 | ATTAGDWSLLDVRGMPTGARVGKG |
| | | CACTGGGGCCAGGGTGGGCAAAGGAGGTTGGTGCCAATGTGGTGCTG | | GWCQCGAGSXFLLGLEHSLPQALS |
| | | GAAGTANACCCCTCCTGGGTCTGGAACATTCTCTGCCCCAGGCTCTT | | TWGCCDNIQLWCLGYLCGDKTDLV |
| | | TCCACCTGGGGATGTTGTGACAACATCCAGCTGTGGTGTCTGGGCTA | | RGWVANTSPGWAEATLLFGPRPGE |
| | | TTTGTGTGGTGACAAGACAGACCTGGTCAGGGGGTGGGTGGCAAACA | | APPFPPLPWPRGHSFLGFV*VFHR |
| | | CCTCACCTGGCTGGGCAGAGGCCACCTTGCTGTTTGGCCCAAGGCCA | | RAAKRYGSWVISVFTLQIFLEIXS |
| | | GGTGAGGCCCCTCCCTTTCCACCATTGCCCTGGCCCCGTGGCCATTC | | S*CYLQGWDWPPCLFQQSGPHPVC |
| | | ATTCTTGGGGTTTGTTTGAGTCTTCCACAGAAGAGCAGCAAAGAGAT | | SCSSPSLCPPSVSWVVSRFAPGLC |
| | | ATGGGAGTTGGGTGATCAGTGTATTTACTTTGCAGATTTTTCTGGAA | | MFHLFSF |
| | | ATAANCTCTAGCTGATGCTATTTGCAAGGCTGGGATTGGCCTCCCTG | | |
| | | TCTTTTCCAACAATCAGGACCCCATCCNGTCTGTTCCTGTTCTTCTC | | |
| | | CCAGCCTCTGCCCTCCTTCTGTNTCCTGGGTGGTCTCCCGNTTCGCT | | |
| | | CCTGGCCTTTGCATGTTCCATCTCTTCTCNTTC | | |
| prey108112 | 542 | TTTTTGCTTTACATTTAAGGCTCTGATCTTGTTTTATGGCTTAGCAT | 543 | FLLYI*GSDLVLWLSIWFIPWALE |
| | | ATGGTTTATTCCATGGGCTCTTGAAAAGAGTATTGTTTTGCTTTCAT | | KSIVLLSWYSSTNVX***QASDLL |
| | | GGTACAGTTCTACAAATGTCANGTGATGATAGTGTTGTTCAGATCTT | | WPC*LFIYLLSQLLRDRCGYL*LC |
| | | CTTTGGCCTTGCTAACTTTTTATCTACTTGCTTTCTCAATTATTGAG | | NRLLQNLLVFVYLL*RIYHYQICF |
| | | AGACAGGTGTGGTTATCTATGACTATGTAACAGACTACTCCAAAACT | | W*LTSHPET**LKIICYCLNNYQF |
| | | TACTAGTGTTTGTTTATTTATTATAAAGGATTTATCATTACCAAATC | | CGFIGLD*AVSCLGSFL*LQSDEG |
| | | TGTTTTTGGTGATTAACAAGCCACCCTGAAACTTGATAGCTTAAGAT | | CGRNHVRLPLDWSSKLISCPFPGV |
| | | AATTTGTTATTGTCTTAATAATTACCAGTTCTGTGGTTTTATTGGGC | | SVGMAGTAGAGQARSPN |
| | | TGGATTGAGCAGTATCTTGCTTGGGGTCTTTCTTGTGATTGCAGTCA | | |
| | | GATGAAGGCTGCGGCCGAAATCATGTGAGGCTGCCTCTGGACTGGTC | | |
| | | TTCTAAGCTGATATCTTGCCCCTTTCCTGGCGTCTCAGTTGGAATGG | | |
| | | CAGGAACAGCTGGGGCTGGCCAGGCCCGCTCCCCGAACC | | |
| prey108115 | 544 | AAACAATGGGAAGCCTNGGAGGTTCTNTNGCTGGGNGTGATAGTGAT | 545 | KQWEAXEVLXLGVIVIKTPAXSLQ |
| | | TAAAACACCAGCTTANTCCTTACAGGACTAGTANGCANGTGAAGGAA | | D*XAXEGTASGYGTDGIQXLPSXL |
| | | CAGCATCNGGATATCGAACAGACGGAATACAACSIACTTCCTTCCCN | | QQEFSLHPQGLQKCLIFPVHLFSI |
| | | GCTCCAGCAGGAATTCTCACTTCACCCTCAGGGACTGCAAAAATGTC | | SFIL*HSESS*ILGEGKPEGGNLG |
| | | TCATTTTCCCCGTTCACTTATTTTCCATTTCTTTCATTTTGTGACAT | | EAHSVTVVQQGWARRRKLSWGVRS |
| | | TCTGAGTCGTCTTAAATCTTGGGGCAGGGAAAGCCTGAAGGGGGTAA | | QGRSPPGGAHSLGEGLVQPGVWWQ |
| | | TCTGGGAGAGGCTCACTCAGTAACTGTGGTCCAACAAGGGTGGGCAA | | LLLXAPAPPSSHISALLXAHTRCA |
| | | GGAGAAGGAAGTTAAGCTGGGGAGTGCGTTCCCAGGGAAGAAGCCCC | | PPQQTPIPEKXKCXXPRXVCA |
| | | CCAGGTGGGGCACACAGCCTAGGGGAAGGGCTGGTGCAGCCTGGGGT | | |
| | | CTGGTGGCAGCTCCTCCTTNTGGCCCCTGCCCCACCTTCCAGCCACA | | |
| | | TTTCCTAGCTGCTTTNTGCCCACACCCGCTGTGCACCACCACAACAA | | |
| | | ACTCCCATTCCTGAAAAGCNCAAGTGTCNTGNGCCTCGANAGGTATG | | |
| | | TGCC | | |
| prey108116 | 546 | TTAACCCTTCTAGGGTTTGTATGAATTCCTAAATTTCCAATTCCTTC | 547 | LTLLGFV*TPKFPIPSPHSQVPTL |
| | | CCCACATTCCCAAGTGCCAACTTTGCTTCATCGATTCAAAATATTAT | | LHRFKILSTICPLLYISNRCSTLS |
| | | CAACTATTTGTCCTTTATTGTACATTTCAAACACATGCAGTACTTTA | | FFSQSDSSF*NIFLFVNISHEENR |
| | | TCATTTTTTTCACAGAGCGATAGCTCTTTTTAAAATATTTTTCTGTT | | VSA*NSGALTLLCLSSFYLP*AQT |
| | | TGTGAACATTTCACATGAGGAAAACAGAGTAAGTGCATGAAACTCGG | | PYQNVFGLRFPFGNFIG*SVPVPG |
| | | GTGCACTTACTCTTTTGTGCCTCTCCTCCTTTTATCTTCCCTAGGCA | | F*NFLGLTQGTHSCQVSWRI**IS |
| | | CAGACACCTTACCAGAATGTCTTTGGATTGAGGTTCCCTTTTGGAAA | | ASGSLPEDNLCLLGSSNLXXXXXX |
| | | TTTTATAGGGTGATCTGTCCCTGTTCCTGGGTTTTAGAACTTCCTGG | | XXXXXXXXXXXISEF*ISLVHL |
| | | GGTTGACCCAGGGTACTCACAGTTGCCAGGTATCTTGGAGGATCTAG | | |
| | | TGAATATCAGCCTCTGGTTCACTCCCAGAGGACAACCTCTGCCTGCT | | |
| | | GGGCTCAAGCAATCTTTNNNNNNNNNNNNNNNNNNNNNNNNNNNNNN | | |
| | | NNNNNNNNNNNNNNNNNNNAATTTCTGAATTTTGAATCAGCCTTGTA | | |
| | | CACCTG | | |
| prey108117 | 548 | GGAAGCTGCAGTGAGCTGAGATTGCACCAAAAGAACCNACATGTACC | 549 | GSCSELRLHQKNXHVPKYQLCTRC |
| | | AAAATACCAGTTGTGTACCAGGTGCTTTGCATATGTNGGTTTGGGAT | | FAYVGLGYIGSEETKLLXSQHGIS |
| | | ATATTGGTTCAGAGGAGACAAAGCTGCTCCNGAGCCAACACGGAATT | | HFIELFSYI*IMSNNEVSFYYFSI |
| | | TCCCACTTCATAGAACTTTTTAGTTATATCTAAATTATGTCCAACAA | | I*I*M*LRVILTQTTYCRVQRMLS |
| | | TGAGGTTTCATTTTACTATTTTAGCATAATATGAATATAAATGTGAC | | ENRSFISSS*KKRGTKNCDWNYVI |
| | | TGAGAGTTATCTTGACCCAGACAACCTACTGCAGAGTCCAAAGGATG | | YVPFYVLR*SSKMMAEY*CLWSRS |
| | | CTAAGTGAAAACAGGTCATTTATATCTAGTTCTTGAAAGAAAAGAGG | | TTDSKSEVTCQSHSFTVVSL |
| | | AACCAAAAACTGTGACTGGAATTATGTGATATACGTTCCTTTTTATG | | |
| | | TTCTGAGGTAGTCATCAAAAATGATGGCAGAATACTGATGTCTATGG | | |
| | | AGCAGAAGTACAACTGACAGTAAGTCAGAAGTTACTTGCCAATCCCA | | |
| | | TTCTTTCACTGTAGTAAGCTTGAC | | |
| prey99362 | 550 | A | 551 | |
| prey108121 | 552 | TTTTTACTGTATTCTTTNATCTGAAAGATCTACTCTATCTTCATTGT | 553 | FLLYSXT*KIYSIFIVLKSLSRKV |
| | | TTTGAAAAGTTTATCTAGAAAAGTCAAGATAAAGAATGAAATATGCA | | KIKNEICNIIYLLLFHFVKVYL*E |
| | | ATATTATATATTTGTTGTTATTTCACTTTGTAAAAGTGTATTTATAA | | S*EAF*TRMIAKTRTEKQLIMGWA |
| | | GAAAGCTGAGAGGCTTTCTACACTAGAATGATAGCAAAAACAAGAAC | | LF*LLYVYYFTYSL**PYVISTNI |
| | | AGAAAAACAACTTATCATGGGCTGGGCACTGTTCTAACTGCTTTATG | | ITILNIK*LRPREIKQLAQYHTHT |
| | | TGTATTATTTCACTTACTCCTTATAATAACCTTATGTAATAAGTACT | | IFCXG |
| | | AATATTATTACCATTTTAAATATAAAATAACTGAGGCCCAGAGAGAT | | |
| | | TAAGCAACTTGCTCAATATCACACACACACAATATTCTGTNGTGGNT | | |
| | | N | | |
| prey108122 | 554 | CCNAGCCTGCCAGCCAGGCCCGAGTCCTCTCCAGCTCAGAGACCCCT | 555 | PSLPARPESSPAQRPLPGPCPSPQ |
| | | GCCAGGACCCTGCCCTTCACCACAGGTGAGACCGGGAGGAGGGTGGC | | VRPGGGWRVEGGARLHESMWVSWC |
| | | GGGTGGAGGGAGGGGCTCGGCTGCACGAGTCCATGTGGGTGTCTTGG | | HLGHF*RPQSA*PC*AHVVPMEHM |
| | | TGTCACTTGGGACATTTTTAGAGGCCACAGAGTGCTTAGCCTTGTTA | | EQLVIAFGR*GPRLTKSKVTSLWS |
| | | GGCCCACGTAGTACCCATGCAGCACATGGAACAGCTGGTTATTGCAT | | NSKW*SSQDKNSSLLPMHSA |
| | | TTGGCCGATGAGGACCAAGGCTCACGAAATCTAAGGTGACAAGCCTG | | |
| | | TGGTCAAATAGCAAGTGGTAGTCAAGCCACGATAAGAATTCCAGTCT | | |
| | | CCTGCCAATGCACTCAGCCC | | |
| prey108123 | 556 | TGGGTATGGATGGGGTGTTTTTAATGTGTTTTAGATAATTTAAATTT | 557 | WVWMGCF*CVLDNLNFALGIGYVA |
| | | TGCCCTAGGTATAGGATATGTTTAAAGCATAAAACAGGTTTGTGTTG | | SIKQVCVGADQHLAWGRRSEEKFH |
| | | GGGCTGATCAACATCTTGCCTGGGGCAGAAGGAGTGAGGAAAAATTC | | SVICLDSWKETYFHLKEDK*XHKI |
| | | CACTCTCTTATATGTTTGGATAGCTGGAAAGAGACTTACTTTCACCT | | IIHRXRGS*R*ST*SFXFLXQRFI |
| | | GAAGGAAGATAAGTAAGANCATAAAATCATTATTCACAGGTANAGAG | | XKXFXTYSKVIXXXHECSHXPXXX |
| | | GATCTTGAAGATAATCTACATAATCCTTTCNCTTTTTANGACAACGA | | ASSXEYXLXFX |
| | | TTTATTTANAAATANTTTTANACTTATAGCAAAGTTATANAANTANT | | |
| | | NCACGAGTGTTCCCATNTGCCATTNANTTNNGCTTCCTCNANAGAAT | | |
| | | ACNCCCTTCNTTTTNAAG | | |
| prey108124 | 558 | ACATACTTTTCCCTAATCCACTTTACTCTCTGCCTGCTACATTCATT | 559 | TYFSLIHFTLCLLHSFPVPDICFI |
| | | CCCAGTGCCAGACATTTGTTTTATCCTTTGGAAGATACATCTCTTCT | | LWKIHLFFSTFLITICLFSTPIKC |
| | | TTTCTACATTCCTGATTACAATCTGTCTTTTCAGTACTCCAATCAAG | | HRPS*SLFAFSRPH*SFFTLNS*S |
| | | TGTCATCGCCCTTCATAAAGCCTCTTTGCTTTCTCTAGACCACATTG | | TDTLNSSCWFPSMYLCSSRKFFLS |
| | | ATCCTTTTTCACTCTTAACTCCTAGAGTACTGATACTTTGAATTCTT | | ASCMGSTMLHIGFIGMNMIDNVPV |
| | | CGTGTTGGTTCCCGAGTATGTATTTGTGTAGCAGTAGGAAGTTTTTT | | LKKFSILVGRDS**IINKKISVRI |
| | | TTGAGTGCCTCTTGTATGGGAAGCACTATGCTACATATTGGGTTTAT | | **KNV*RMNQIS*LK*QS |
| | | AGGAATGAACATGATAGATAATGTCCCTGTCCTTAAGAAGTTTTCTA | | |
| | | TTCTTGTGGGGAGAGACAGCTAATAAATAATTAACAAGAAAATATCA | | |
| | | GTAAGAATCTGATGAAAAAATGTATGAAGAATGAATCAGATCTCTTA | | |
| | | GTTGAAATGACAAAGCCG | | |
| prey56846 | 560 | GAAGACCTTCCACGAGGCCAGCGAGGACTGCATCTCGCGCGGGGGCA | 561 | KTFHEASEDCISRGGTLSTPQTGS |
| | | CCCTGAGCACCCCTCAGACTGGCTCGCAGAACGACGCCCTGTATGAG | | ENDALYEYLRQSVGNEAEIWLGLN |
| | | TACCTGCGCCAGAGCGTGGGCAACGAGGCCGAGATCTGGCTGGGCCT | | DMAAEGTWVDMTGARIAYKNWETE |
| | | CAACGACATGGCGGCCGAGGGCACCTGGGTGGACATGACCGGCGCCC | | ITAQPDGGKTEWCAVLSGAANGKW |
| | | GCATCGCCTACAAGAACTGGGAGACTGAGATCACCGCGCAACCCGAT | | FDKRCRDQLPYICQFGIV* |
| | | GGCGGCAAGACCGAGAACTGCGCGGTCCTGTCAGGCGCGGCCAACGG | | |
| | | CAAGTGGTTCGACAAGCGCTGCCGCGATCAGCTGCCCTACATCTGCC | | |
| | | AGTTCGGGATCGTGTAG | | |
| prey108133 | 562 | T | 563 | |
| prey108134 | 564 | TGGAGGTACTATGGAATTATATAATGTAATTCAAAAGAAGGCAGAAA | 565 | WRYYGIA*CNSKSGRKGPKNRRDK |
| | | AGGACCTAAGAACAGAAGGGACAAATAGAAAACAGCCAATCTATTTA | | *KTANLFKHMFSIWKTVLNRNGLN |
| | | AACACATGTTTTCTATATGGAAAACAGTATTAAATAGAAATGGCCTA | | T*KAEVDRVNLKSIPNY*YMQAKK |
| | | AACACTTAAAAAGCAGAAGTTGACAGAGTGAATCTAAAAAGCATACC | | ILLYIGKQRGHK*SM*KDITMQTL |
| | | CAACTATTAATACATGCAAGCTAAAAAAATCCTACTTTACATAGGAA | | IFYKKND*HQALSTLDSNFLGVKQ |
| | | AACAGAGAGGTCATAAGTAAAGCATGTGAAAAGATATCACAATGCAA | | VF*IQSGCKTA*F |
| | | ACACTAATTTTTTATAAAAAGAATGATTAACACCAAGCACTATCAAC | | |
| | | TTTGGACAGTAATTTCCTAGGTCTTAAACAAGTTTTCTGAATACAAT | | |
| | | CTGGATGCAAAACGGCCTGATTTG | | |
| prey92918 | 566 | ATGAACTCCATGAGTCTCTCCAGGGCTGCCTGCAGCACGTCTTTTCC | 567 | MNSMSLSRAACSTSFPSSLFGFPS |
| | | AAGTAGCCTATTTGGATTCCCATCTCAAATGTCCTGGATGCGAGCGT | | QMSWMRASAAPELGAGEVPFGEPF |
| | | CAGCGGCTCCACAGCTCGGGGCGGGTGAGGTCCCCTTTGGGGAACCC | | PGHRGRGAAVCGQEDPRGSQERRS |
| | | TTTCCTGGCCATCGAGCTCGGGGGGCTGCCGTCTGTGGGCAGGAGGA | | LHCEKLPGCSAFSFYHGKCRLGPW |
| | | CCCGAGGGGCAGCCAGGAAAGGCGATCTCTTCACTGTGAAAAGTTGC | | GEPAGLWCCPRPPPPPECSSSLGS |
| | | CCGGGTGCAGCGCCTTTTCCTTCTACCATGGGAAATGCAGGCTGGGC | | HKQATQEEQNTASVDFPNFPGK* |
| | | CCTTGGGGTCAGCCTGCGGGGCTCTGGTGCTGTCCCCGACCCCCACC | | |
| | | ACCACCAGAATGCAGTTCCAGCTTAGGAAGCCACAAACAACCCACCC | | |
| | | AGGAGGAACAAAACACCGCCAGCGTGGATTTTCCAAATTTCCCTGGA | | |
| | | AAGTAA | | |
| prey108139 | 568 | TCTTGGCTGGGTATAATACTCTTAGATTTATTTTCTTCCCCAACACT | 569 | LAGYNTLRFIFFPNTLQLSFHHPL |
| | | TTGCAGCTGTCATTCCACCATCCCCTGGCTCACATGTGCTGTGGGGA | | AHMCCGEA*GQLIFPHVEACPSLH |
| | | ACCCTGAGGTCAGCTGATTTTTCCCCATGTGGAGGCTTGTCCTTCCC | | EFLRNSFFLSHSSSA*PD |
| | | TTCATGAATTTCTGAGGAATTCTTTCTTTTTGTCTCATAGTTCTTCT | | |
| | | GCTTAACCAGACC | | |
| prey108142 | 570 | TTGTCTTTTGCTCATCTTTCCNATATCNTTCTTTACTCTCTTCTATA | 571 | LSFAHLSXIXLYCLL*LLHPH*E* |
| | | ACTTCTTCACCCCCACTAAGAATAACTGTTTGTTTGTTTCCCAAATA | | LFVCFPNISCFCT*VVIWITFA*K |
| | | TTAGTTGTTTCTGCACTTGAGTGGTGATTTGGATCACNTTTTAAAAA | | KSGMLGTCCFCSPTNF*QIL*GSF |
| | | AAATCTGGAATGCTAGGCACNTGTTGCTTTTGTTCGCCTACAAATTT | | QCGPFFNI*LSAKL*ALPYVLRSG |
| | | CTAACAAATTCTTTAAGGCTCATTTCAGTGTGGCCCATTCTTCAATA | | WTPFYLINNNYYLFSYXASXSRLS |
| | | TTTAACTCTCTGCAAAGTTATGAGCTCTGCCGTATGTTCTACGGTCA | | QQLXNWDSLLLIFX |
| | | GGCTGGACTCCCTTCTATTTAATAAATAATAATTATTATTTATTTTC | | |
| | | ATATTNGGCCTCTNACAGCAGACTTTCCCAACAACTGGNAAACTGGG | | |
| | | ATTCACTCTTACTGATTTTCGNC | | |
| prey108143 | 572 | ACTTATTGCCAAGTTTTGGCAGTNNGCTTTACATTCTCTAAACATGG | 573 | TYCQVLAVXFTFSKHGXGRYFLGE |
| | | AANGGGAAGATACTTCCTTGGAGAACCTGACTGATCTATGCAAAANA | | PD*SMQXTDRH*WLGSASRTSVQ* |
| | | CCGATAGACACTGATGGCTGGGGTCTGCCAGCAGGACTTCTGTGCAG | | XPLVHKPHPWTLTFLPPFXVFIFN |
| | | TGAANCCCACTTGTTCACAAGCCCCACCCATGGACACTGACTTTCTT | | MSNNQ*SPDI*SNSEAKTNLAKTR |
| | | GCCACCTTTTTNGGTCTTCATCTTCAATATGAGTAATAACCAGTAAT | | ILS*RDDEGRRKLKNIIMDILRKI |
| | | CCCCTGATATTTAAAGCAATTCAGAAGCCAAAACAAACCTAGCGAAA | | REYTVIRKSV*TLKEKEALGSKNV |
| | | ACTAGGATATTGTCATAAAGAGACGATGAAGGGAGAAGAAAACTTAA | | LLKNGTDRRNKKLKMFFEYKEEEA |
| | | GAATATTATCATGGATATTCTCAGAAAGATCAGAGAATATACTGTAA | | TQKVEKKITK*KT |
| | | TCAGAAAATCAGTATAGACNCTAAAAGAAAAGGAAGCTTTAGGTTCT | | |
| | | AAAAATGTACTCCTGAAAAATGGAACTGATAGAAGAAATAAGAAACT | | |
| | | CAAAATGTTTTTTGAATATAAAGAAGAGGAAGCTACCCAGAAAGTAG | | |
| | | AAAAAAAAATTACAAAATGAAAAACC | | |
| prey108157 | 574 | GTATTTTTCTATTCTTTAGAAATTTTTTTATAAGACGATNATTTCAA | 575 | VFFYSLEIFL*DDXFKPXXINPYV |
| | | ACCTATNTNAATAAATCCTTATGTATTTATCATTTGACTTTATTATA | | FII*LYYKLIINLVSSLSTPSSPS |
| | | AACTCATAATCAATCTTGTTTCATCTCTTTCCACTCCTTCCTCCCCT | | SSPTKVSKCIRXVVYXXFSVYLAK |
| | | TCTTCCTCCCCCACTAAAGTTTCCAAGTGTATCCGANGTGTTGTTTA | | VRAXXXXXXXXXXXXXXXXXXXCL |
| | | TNACCNTTTCAGTGTGTACCTTTAAAAGGTAAGGGCNNNNNNNNNNN | | XXXXXXXXXXXXXSPTNKYXLGLG |
| | | NNNNNNNNNNNNNNNNNNNNNNNNNNNNNNNNNNNNNNNNNNNNNNA | | GACL |
| | | TTGCTTGAANNNNNNNNNNNNNNNNNNNNNNNNNNNNNNNNNNNNCT | | |
| | | CACCTACAAACAAATATNAACTGGGCCTGGGTGGTGCATGCCTNTG | | |
| prey108159 | 576 | T | 577 | |
| prey108160 | 578 | TTTCTATGTTATTTATAAAATTTTTTACTGAAAATGTTTCTCAGAGA | 579 | FLCYL*NFLLKMFLRDVLDFI*LS |
| | | TGTTCTGGATTTTATCTGACTCAGTTTTTCTAGTTTTTATTCAATGA | | FSSFYSMIIKRPKEKCHRQCELET |
| | | TCATAAAGAGGCCTAAGGAAAAATGCCACAGGCAATGTGAGTTAGAA | | LRFHKRLPSFKNTCXXXXXXXXXX |
| | | ACTCTCAGGTTTCACAAAAGGCTCCCATCATTTAAAAACACTTGTAN | | XXXXXXXXXXXXXXXXXXXXXXXX |
| | | NNNNNNNNNNNNNNNNNNNNNNNNNNNNNNNNNNNNNNNNNNNNNNN | | XXXXXXXXXXXXXXXXXXXXACSA |
| | | NNNNNNNNNNNNNNNNNNNNNNNNNNNNNNNNNNNNNNNNNNNNNNN | | TNSGSQGERILEP |
| | | NNNNNNNNNNNNNNNNNNNNNNNNNNNNNNNNNNNNNNNNNNNNNNN | | |
| | | NNNNNNNNNNNNNNNNNNTGCCTGTAGTGCCACTAATTCAGGAAGCC | | |
| | | AAGGTGAGAGGATCCTTGAGCCCAG | | |
| prey56846 | 580 | GAAAGATGTTGTGAACACAAAGATGTTTGAGGAGCTCAAGAGCCGTC | 581 | KDVVNTKMFEELKSRLDTLAQEVA |
| | | TGGACACCCTGGCCCAGGAGGTGGCCCTGCTGAAGGAGCAGCAGGCC | | LLKEQQALQTVCLKGTKVHMKCFL |
| | | CTGCAGACGGTCTGCCTGAAGGGGACCAAGGTGCACATGAAATGCTT | | AFTQTKTFHEASEDCISRGGTLST |
| | | TCTGGCCTTCACCCAGACGAAGACCTTCCACCAGGCCAGCGAGGACT | | PQTGSENDALYEYLRQSVGNEAEI |
| | | GCATCTCGCGCGGGGGCACCCTGAGCACCCCTCAGACTGGCTCGGAG | | WLGLNDMAAEGTWVDMTGARIAYK |
| | | AACGACGCCCTGTATGAGTACCTGCGCCAGAGCGTGGGCAACGAGGC | | NWETEITAQPDGGKTENCAVLSGA |
| | | CGAGATCTGGCTGGGCCTCAACGACATGGCGGCCGAGGGCACCTGGG | | ANGKWFDKRCRDQLPYICQFGIV* |
| | | TGGACATGACCGGCGCCCGCATCGCCTACAAGAACTGGGAGACTGAG | | |
| | | ATCACCGCGCAACCCGATGGCGGCAAGACCGAGAACTGCGCGGTCCT | | |
| | | GTCAGGCGCGGCCAACGCCAAGTGGTTCGACAAGCGCTGCCGCGATC | | |
| | | AGCTGCCCTACATCTGCCAGTTCGGGATCGTGTAG | | |
| prey78738 | 582 | ATGGGGCAAGCTACGTTACATTATGGCAGAAAAAAAGTGCAACTGAC | 583 | MGQATLHYGRKKVQLTYYKEIF*V |
| | | ATACTACAAAGAGATTTTTTAAGTTTAAAAAAAGTTTGGATCTTTTG | | *KKFGSFGFFFFFGXXXXKKPLXX |
| | | GATTTTTTTTTTTTTTTGGGCNTTNANGGNCTAAAAAACCCCTAANT | | NXPXXXGXFFFFFXXXPKXPGGGF |
| | | NATAATANCCCCCNCAANTANGGANGNTTTTTTTTTTTTTTTNGGNC | | FXXPPFPXXXGPXPQXTPXNXPXX |
| | | CAANCCCAAAAANCCNGGTGGGGGTTTTTTCCNTTNCCCCCCCTTCC | | GXFXXTXXXKPGPXGXXGNPXPFG |
| | | CGNNTTTNNNGGGTCCNNNTCCCCAANGNACCCCCCNTAATTTNCCC | | LEKPXPQKNPXFXKTXF |
| | | CNCNGGGGGGNNTTTNGTNAAACCNCCNAAANAAAACCGGGCCCAAN | | |
| | | GGGGGANNAGGGGAACCCTNGCCCNTTTGGTTTGGAAAAACCNCNCC | | |
| | | CCCAAAAAAACCCCANTTTTTNAAAAACTTNTTTTTC | | |
| prey78741 | 584 | CTTCCTATGCTAAATGTNAGACCCTTTTCTCTTTTGCAAGCCTAATA | 585 | LPMLNVRPFSLLQAAYXCKEKHWE |
| | | CANATGCAAAGAGAAGCACTGGGAATCTACAGTAAACTGCCTTGTTT | | ASTVNCLVYMPNIRSLFKNVSAKQ |
| | | ATATGCCCAACATTAGATCTTTGTTTAAAAATGTTTCTGCGAAACAA | | TVTDFLCQFFCDHXHFLYKHDNLY |
| | | ACTGTGACAGACTTTTTATGTCAGGAATTCTGTGACCATTANCATTT | | QKMAAWERXATISXSSYEHEAMVR |
| | | TCTATACAAGCATGATAATCTTTACCAAAAGATGGCTGCTTGGGAAA | | LPRXTEXFCSRWTLXGLP*KERLA |
| | | GAANCGCCACTATTAGCANCTCATCCTATGAACACGAGGCGATGGTC | | TAEPLLXPESR |
| | | AGGTTACCCAGGNGCACAGAANGGTTCTGTTCTAGGTGGACACTCAG | | |
| | | NCGTCTACCCTGAAAGGAGAGACTGGCCACAGCAGAGCCGCTGCTGA | | |
| | | NACCTGAAAGCAGA | | |
| prey78742 | 586 | NNNNNNNNNNNNNNNNNNNNNNNNNNNNNNNNNNNNNNNNNNNNNNN | 587 | XXXXXXXXXXXXXXXXXXXXXQKT |
| | | NNNNNNNNNNNNNNNCCAAAAAACCANAGGTCTTNGNGGTTTGTTGA | | XGLXGLLKGPTXGKLETXYKRVVE |
| | | AGGGCCCTACANAAGGGAAATTGGAGACTTNTTACAAAAGAGTAGTG | | DLSWGXXCIGKKKTWWRLEIQGHG |
| | | GAAGATTTGTCTTGGGGCTTNTNCTGTATAGGGAAAAAAAAAACCTG | | LVYF*PFCX*PP**XTXFTLXPXN |
| | | GTGGAGATTGGAGATTCAAGGTCATGGTTTAGTATATTTTTAACCTT | | XCMXN*XXRXITPXLFAVXAGIPI |
| | | TTTGTNTGTAGCCCCCTTAGTAANAAACNCNTTTTACNTTGTGNCCC | | F*KKK*YGSWLSILTS*IVR |
| | | NGTAATNAATGTATGNATAACTGANATAAAAAAGGTTNATAACCCCN | | |
| | | TTNTTGTTTGCAGTCCNTGCTGGGATTCCTATTTTTTAAAAAAAAAA | | |
| | | ATAGTATGGGTCATGGCTCAGTATATTGACTTCATGAATAGTTAGGC | | |
| | | C | | |
| prey78744 | 588 | TCATCNTCGACTGNNGTACANGAAATCTCTCAACAAACANCTGTGGN | 589 | SSSTXVXEISQQTXVXPESD*XXE |
| | | GCCAGAATCTGATTGAANNAGNGAGGTTGNTAGGACTCACNATCCAN | | VXRTHXPXXP*XCIXNQVKVCYIC |
| | | ATGANCCTTGATNCTGCATTNGTAATCAGGTAAAAGTCTGTTATATC | | KSII*IN*KEENYFIFKHFFKLT* |
| | | TGTAAAAGTATAATCTGAATAAACTAGAAGGAAGAGAACTATTTCAT | | NTFALFVYFSPPSYKSDFCCKY*V |
| | | TTTTAAGCACTTTTTTAAACTCACTTAAAATACCTTTGCTTTATTTG | | *RKMLPIILALKYDR*XNXCWCTT |
| | | TATACTTTTCTCCCCCTTCTTACAAAAGTGACTTTTGCTGTAAATAC | | TRYLRCTLAALXKY |
| | | TGAGTATAAAGAAAAATGTTACCCATAATCCTNGCCCTCAAATACGA | | |
| | | CCGGTAANTNAACNTTTGTTGGTGTACCACTACCAGATACCTGAGGT | | |
| | | GCACNTTGCCNGCCTTAANAAAATACAG | | |
| prey3062 | 590 | ATGGGCGACAAAGGGACGCGAGTGTTCAAGAAGGCCAGTCCAAATGG | 591 | MGDKGTRVFKKASPNGKLTVYLGK |
| | | AAAGCTCACCGTCTACCTGGGAAAGCGGGACTTTGTGGACCACATCG | | RDFVDHIDLVDPVDGVVLVDPEYL |
| | | ACCTCGTGGACCCTGTGGATGGTGTGGTCCTGGTGGATCCTGAGTAT | | KERRVYVTLTCAFRYGREDLDVLG |
| | | CTCAAAGAGCGGAGAGTCTATGTGACGCTGACCTGCGCCTTCCGCTA | | LTFRKDLFVANVQSFPPAPEDKKP |
| | | TGGCCGGGACGACCTGGATGTCCTGGGCCTGACCTTTCGCAAGGACC | | LTRLQERLIKKLGEHAYPFTFEIP |
| | | TGTTTGTGGCCAACGTACAGTCGTTCCCACCGGCCCCCGAGGACAAG | | PNLPCSVTLQPGPEDTGKACGVDY |
| | | AAGCCCCTGACGCGGCTGCAGGAACGCCTCATCAAGAAGCTGGGCGA | | EVKAFCAENLEEKIHKRNSVRLVI |
| | | GCACGCTTACCCTTTCACCTTTGAGATCCCTCCAAACCTTCCATGTT | | RKVQYAPERPGPQP |
| | | CTGTGACACTGCAGCCGGGGCCCGAAGACACGGGGAAGGCTTGCGGT | | |
| | | GTGGACTATGAAGTCAAAGCCTTCTGCGCGGAGAATTTGGAGGAGAA | | |
| | | GATCCACAAGCGGAATTCTGTGCGTCTGGTCATCCGGAAGGTTCAGT | | |
| | | ATGCCCCAGAGAGGCCTGGCCCCCAGCCC | | |
| prey37079 | 592 | ATGCTATCACATAATACTATGATGAAGCAGAGAAAACAGCAAGCAAC | 593 | MLSHNTMMKQRKQQATAIMKEVHG |
| | | AGCCATCATGAAGGAAGTCCATGGAAATGATGTTGATGGCATGGACC | | NDVDGMDLGKKVSIPRDIMLEELS |
| | | TGGGCAAAAAGGTCAGCATCCCCAGAGACATCATGTTGGAAGAATTA | | HLSNRGARLFKMRQRRSDKYTFEN |
| | | TCCCATCTCAGTAACCGTGGTGCCAGGCTATTTAAGATGCGTCAAAG | | FQYQSRAQINHSIAMQNGKVDGSN |
| | | AAGATCTCACAAATACACATTTGAAAATTTCCAGTATCAATCTAGAG | | LEGGSQQAPLTPPNTPDPRSPPNP |
| | | CACAAATAAATCACAGTATTGCTATGCAGAATGGGAAAGTGGATGGA | | DNIAPGYSGPLKEIPPEKFNTTAV |
| | | AGTAACTTGGAAGGTGGTTCGCAGCAAGCCCCCTTGACTCCTCCCAA | | PKYYQSPWEQAISNDPELLEALYP |
| | | CACCCCAGATCCACGAAGCCCTCCAAATCCAGACAACATTGCTCCAG | | KLFKPEGKAELPDYRSFNRVATPF |
| | | GATATTCTGGACCACTGAAGGAAATTCCTCCTGAAAAATTCAACACC | | GGFEKASRMVKFKVPDFELLLLTD |
| | | ACAGCTGTCCCTAAGTACTATCAATCTCCCTGGGAACAAGCCATTAG | | PRFMSFVNPLSGRRSFNRTPKGWI |
| | | CAATGATCCGGAGCTTTTACAGGCTTTATATCCTAAACTTTTCAAGC | | SENIPIVITTEPTDDTTVPESEDL |
| | | CTGAAGGAAAGGCAGAACTGCCTGATTACAGGAGCTTTAACAGGGTT | | * |
| | | GCCACACCATTTGGAGGTTTTGAAAAAGCATCAAGAATGGTTAAATT | | |
| | | TAAAGTTCCAGATTTTGAGCTACTATTGCTAACAGATCCCAGGTTTA | | |
| | | TGTCCTTTGTCAATCCCCTTTCTGGCAGACGGTCCTTTAATAGGACT | | |
| | | CCTAAGGGATGGATATCTGAGAATATTCCTATAGTGATAACAACCGA | | |
| | | ACCTACAGATGATACCACTGTACCAGAATCAGAAGACCTATGA | | |
| prey79756 | 594 | GCGGGGCAAGCTACGTTACATTATGGCAGGAAAAAAAGTGCAACTGA | 595 | AGQATLHYGRKKSATDILQRDFLX |
| | | CATACTACAAAGAGATTTTTTAANGTTTAAAAAAAGTTTGGATCTTT | | EAKKSLDLLDFFFFXXXXA*KXXX |
| | | TGGATTTTTTTTTTTTTNGGGNNTTNNGGGCCTAAAAAANCNCTAAN | | *XKXPXXXXXFFFFXGPXXKXXXX |
| | | TAANAAAAANCCCCCNNAAANNNGGNNNNTTTTTTTTTTTTTTNNGG | | XFSXXPXXXXXXXXXPKXPXLSXP |
| | | CCCNNCCCNAAAACNNCNGGNANGGNTTTTTTCCNTNNNCCCNCNTT | | XXXXXVKXPXKNXGGXGTMGXXXX |
| | | NCNGNNTTTGNCNNNNCCNCNTCCCNAAGGNNCCCCNCCTTAGTNNC | | FVXKKXXXPKTLF |
| | | CCNNNNNGGGNNNCNTTNGTNAAANCNCCNNANAAAAACNGGGGGGG | | |
| | | ANGGGGGACNATGGGAANCCNTNCCCNTTTTGTTTNNAAAAAACNGN | | |
| | | CCCNCCCNAAAACCCTTTTT | | |
| prey78757 | 596 | GGAGTCTCGTTCTGTTGCTCACGCCGGACTGCAGTGGCGCTATCTCG | 597 | GVSFCCSRRTAVALSRLTVSSTSR |
| | | GCTCACTGTAAGTTCCACCTCCCGGGTTCATGCCATTCTCCTGCCTC | | VHAILLPQPPEQLGLQVPATAPG* |
| | | AGCCTCCTGAGCAGCTGGGACTACAGGTGCCCGCCACCGCGCCTGGC | | FFVFLVETGFHRVSQDGLALLTS* |
| | | TAATTTTTTGTATTTTTAGTAGAGACGGGGTTTCACCGTGTTAGCCA | | SGRLGLXKVLGLQA*AAALGLNNQ |
| | | GGATGGTCTAGCTCTCCTGACCTCGTGATCCGGCCGCCTCGGCCTCN | | FFNLTLCPYWDR*CKIIAVIIDHV |
| | | CAAAAGTGCTAGGATTGCAGGCATGAGCCGCCGCGCTTGGCCTAAAT | | MRNHDNLEHQVTQVTFFSYGHLGF |
| | | AATCAGTTCTTTAATCTCACTCTCTGCCCATACTGGGATAGGTAGTG | | RISFFLVQICLPAIGKVTAS*CSR |
| | | CAAAATCATTGCAGTCATAATTGATCATGTCATGAGAAATCATGATA | | V*QIYFIYLFIFFGDRVLLCCPGW |
| | | ACTTAGAACACCAAGTGACACAAGTGACATTTTTTTCTTATGGTCAC | | SAVVRSQLPPPSGFK*FSCLGFPG |
| | | CTTGGCTTCAGAATATCTTTTTTCCTTGTACAAATATGTTTGCCATA | | SWDYRRPPPCPANFFCRDWVSLCW |
| | | AATAGGTAAAGTGACAGCCTCTTGATGCTCAAGAGTCTAGCAGATTT | | PGWF*TPDLVIRLSRPPRAGDYRH |
| | | ATTTTATTTATTTATTTATTTTTTTTGGAGACAGAGTCTTGCTGTGT | | GPLHLAFFCFFETES*SYHPGWSA |
| | | TGCCCAGGCTGGAGTGCAGTGGTGCGATCTCAGCTCCCTCCACCTTC | | VARSWLSATSASWAQAVLLLSLLS |
| | | CGGGTTCAAGTGATTCTCCTGCCTTGGCTTCCCAGGTAGCTGGGACT | | SWDYRCVPPRLASFCIFSGDGVSP |
| | | ACAGGCGCCCACCACCATGCCCAGCTAATTTTTTTTGTAGAGACTGG | | CWPCWSRAPDLR*YTRLGLPKCWN |
| | | GTTTCACTATGTTGGCCAGGCTGGTTTTGAACTCCTGACCTCGTGAT | | YRCEPLCPACKFSYKK*TRKIGQV |
| | | CCGCCTGTCTCGGCCTCCCAGAGCGGGGGATTATAGGCATGGGCCAC | | *KLQVEYISNPKSSNL*N*AIATL |
| | | TGCACTTGGCCTTTTTTTGTTTTTTTGAGACAGAGTCTTGATCTTAT | | QFKNP*PTRMVGYGQNTGTQHYSV |
| | | CACCCAGGCTGGAGTGCAGTGGCACGATCTTGGCTCAGCGCAACCTC | | SLRKK*LTFRVHVYEA*MNFLYRP |
| | | TGCCTCCTGGGCTCAAGCGGTTCTCCTGCTCAGCCTTCTGAGTAGTT | | RSHPQSVMYNHLTPTSEHISS*VF |
| | | GGGACTATAGGTGTGTGCCACCACGTCTGGCTAGTTTTTGTATTTTT | | QIRDTYPVYSRLDFCFSLPQDTGN |
| | | AGTGGAGATGGGGTTTCACCATGTTGGCCATGCTGGTCTCGAGCTCC | | LGQ*KILDRFKNTTKLYMKQEPLE |
| | | TGACCTCAGGTGATACACCCGCCTTGGCCTCCCAAAGTGCTGGAATT | | |
| | | ACAGGTGTGAGCCACTGTGCCCGGCCTGCAAATTTTCTTATAAAAAG | | |
| | | TAAACTAGGAAAATAGGGCAAGTATAAAAATTGCAGGTTGAGTATAT | | |
| | | TTCTAATCCAAAAAGCTCTAACCTCTGAAACTGAGCTATGACACTAC | | |
| | | AATTTAAAAATCCCTAGCCTACCCGCATGGTGGGTTATGGTCAAAAT | | |
| | | ACAGGTACACAGCACTATTCAGTGTCCCTGAAGAAGAAATAGTTAAC | | |
| | | CTTCAGGGTACATGTATATGAAGCATAAATGAATTTCCTGTATAGAC | | |
| | | CTAGGTCCCATCCCCAAAGTGTCATGTATATCCACTTAACTCCAACA | | |
| | | TCAGAACACATCTCGTCCTAAGTGTTTCAGATAAGAGATACTTATCC | | |
| | | TGTGTATAGTAGGTTGGACTTCTGTTTCTCTCTACCACAAGATACAG | | |
| | | GTAATTTGGGGCAGTAAAAGATCCTGGATAGATTTAAAAATACTACA | | |
| | | AAGCTGTACATGAAACAGGAACCCCTCGAGG | | |
| prey78758 | 598 | TGTGTGTGTGTGTGTGTGCGCAAGACACATATGTGTGTGTGTGTGTG | 599 | CVCVCVRKTHMCVCVCLGEHAE*X |
| | | TCTNGGTGAACACTGAGAATGANAANACCCAACNTGCCCCCGNGGAA | | XPTCPRGTAPLXXXTRXXVTFSLH |
| | | CTGCNCCTCTGTNATANNCCACCAGGTNANNNGTCACATTTTCACTN | | KNRXLXXNSXVXKLXIXGXSXCYX |
| | | CATAAAAACAGANCTCTGNCCNATAACTCANATGTTGNNAAATTANT | | DXXLXSSXXPXFXFXK*XSVAGXE |
| | | AATATNCGGANACTCCNCTTGTTATTGNGACTNTNATCTAANCAGCT | | ILXXXXFIEXAAGGRXXYNXHTTI |
| | | CTGNAAANCCNNTTTTCANATTTTNAAAATAATANTCTGTAGCAGCG | | XPSNPL |
| | | GGNGANGAAATCTTAANGNNTNNNNNATTTATTGAAANAGCCGCAGG | | |
| | | GGGGCGGNCNNGTTACAATNCACACACCACTATANNACCGTCNAACC | | |
| | | CCCTTNC | | |
| prey78599 | 600 | CCAGTCCAACGCCCAGAAGATAATCCCACCTCTGTTCAGCTACCGCC | 601 | QSNAQKIIPPLFSYRLAQQQLKEM |
| | | TTGCCCAGCAGCAGTTAAAGGAGATGAAAAAGAAAGGTCTGACGGAA | | KKKGLTETTKVYHVSQSPLTDTAI |
| | | ACTACCAAAGTGTATCACGTGTCTCAGAGTCCCCTGACAGACACTGC | | DAAPSAPLDLESPEEAAANKFRIK |
| | | CATTGATGCGGCCCCCAGCGCTCCCTTAGATTTGGAAAGCCCGGAAG | | SPFPEQPAVSAGERPPSRLDLNVT |
| | | AAGCTGCAGCAAACAAGTTCCGGATCAAATCCCCATTTCCGGAGCAG | | QASCAISPSQTLIRKSQARHVGSR |
| | | CCCGCGGTCAGTGCCGGGGAAAGGCCTCCCTCCAGGCTGGATCTAAA | | GGPSERSHARN |
| | | TGTGACTCAGGCCAGTTGTCCCATAAGCCCCAGCCAGACTCTGATCC | | |
| | | GCAAGTCACAGGCAAGGCACGTGGGCAGCAGAGGGGGCCCGTCCGAA | | |
| | | AGGAGCCATGCCAGGAAC | | |
| prey78760 | 602 | GACATTGTTGTACTTCAAAGGAATCAGACATTTCAGTTTTATGGCAT | 603 | DIVVLQRNQTFQFYGIL*KWVSVC |
| | | TTTGTGAAAATGGGTAAGTGTGTGCAGGAGAAACCAGTGTTTTAGTT | | RRNQCFS*HCYSFWTQCVKQV*NT |
| | | AACATTGTTATAGTTTTTGGACTCAGTGTGTCAAGCAGGTATAAAAT | | L*KEGRKMTNPIIKYDFCCVKLSF |
| | | ACTTTGTAGAAAGAAGGGAGGAAAATGACTAATCCCATTAAATATGA | | KITAVRVVNRFEIF*LHSELIGIL |
| | | CTTCTCCTGTGTGAAGCTTTCTTTTAAGATCACGGCAGTAAGAGTAG | | GSIPPFM*VF*GS*ALSAWILTST |
| | | TAAATAGATTTGAAATCTTCTGACTTCATTCAGAATTAATAGGTATA | | IYSSTYVCFSPXSLIX |
| | | TTAGGAAGTATTCCCCCCTTTATGTGAGTCTTTTGAGGAAGTTGAGC | | |
| | | CATAAGTGCATGGATTCTGACCTCAACAATATATAGTTCTACCTACG | | |
| | | TCTGCTTTTCTCCCANNTCTTTGATATTNN | | |
| prey78761 | 604 | NGACCNCCCNCAGANATTTNTGGTAATNNGGACNTTAATACATTTGN | 605 | XPPXXIXGNXDXNTFXMXLXKGXY |
| | | TATGNNACTGNATAAGGGGANNTACACGGNTACCCTNGAATAACNCC | | TXTLE*XQXWGXDPCTXXXXPVXL |
| | | AGANCTGGGGCNCTGACCCCTGTACAGNTAANNATNTNCCTGTNNCT | | DSSSTYQLMAYYSLKALQIT*TVD |
| | | TTGGACTCCTCTAGTACTTACCAACTAATGGCCTACTATTCACTGAA | | *YIKRLVSTCFYAIMTYSSFLIFS |
| | | AGCCTTACAGATAACATAAACAGTTGATTAATACATAAAAAGACTAG | | IFXRLHSLQVSYSXPKYLLKNTCV |
| | | TATCTACATGTTTTTATGCAATCATGACATATTCATCTTTCTTAATT | | *XHPA*IKPMXXKGX |
| | | TTTTCAATATTTNCTAGGCTACATAGTTTGCAAGTTTCATATTCTTT | | |
| | | NCCAAAATATTTATTGAAAAATACTTGTGTATAAGNGCACCCTGCNT | | |
| | | AGATCAAACCTATGNTGNTCAAGGGTTTNNT | | |
| prey78763 | 606 | GATTCCTGTATCTACCCTGGAATGTAGGCACTCCCCAGGAGTCTGCC | 607 | IPVSTLECRHSPGVCPVHSCSYLD |
| | | CTGTTCACTCTTGCTCATATCTGGACAATTACATCTTCTACTGTTGG | | NYIFYCWL*GSLSSLNYSSSSFAY |
| | | CTGTAAGGATCCCTCAGTTCCCTAAACTATAGCAGTTCCAGCTTTGC | | SPKL*NLIFLL*VDHGHIVSTCLG |
| | | ATACTCTCCTAAGCTCTAGAACCTTATTTTTCTTCTATAGGTGGACC | | LSSFYSPTPIENLLFLTVYLISFK |
| | | ATGGACACATAGTCTCAACATGTCTAGGACTGAGCTCATTCTATTCC | | CFTILLFIRAKDLKLSLILSSHIC |
| | | CCCACTCCCATTGAAAATTTGCTGTTCCTTACTGTATACCTTATCTC | | TTSNHLSSHGSSIHL*YL*DGLAS |
| | | ATTTAAGTGCTTCACCATCCTTCTATTCATCAGGGCTAAAGACCTCA | | FVSTTISLVISCFV*IATS*TLSF |
| | | AATTGTCTTTGATTCTCTCTTCCCACATATGCACCACATCCAATCAT | | CPFPLQFICHVYFIMT*MCHSTDW |
| | | TTGTCAAGTCATGGCAGTTCCATTCATTTGTAATATTTGTAAGATGG | | KS*MMLDKLY*KGLAQKPPPL*IF |
| | | ATTAGCCTCTTTTGTTTCCACTACCATTAGCCTTGTCATCTCTTGTT | | STLIIHPTFMIP |
| | | TTGTTTAAATAGCTACATCTTAGATCCTTTCCTTTTGCCCCTTTCCT | | |
| | | CTCCAGTTCATTTGTCACGTTTATTTCATAATGACATGAATGTGTCA | | |
| | | CTCCACTGATTGGAAGTCTTGAATGATGCTCGATAAGCTTTATTAAA | | |
| | | AAGGCCTGGCTCAAAAGCCACCTCCCCTGTGAATCTTCTCCACTCTA | | |
| | | ATAATTCACCCCACCTTCATGATCCCAC | | |
| prey78764 | 608 | GCCGGGCAAGCTACCTTACATTATGGCAGAAAAAAAGTGCAACTGAC | 609 | AGQATLHYGRKKVQLTYYKEIF*V |
| | | ATACTACAAAGACATTTTTTAAGTTTAAAAAAAGTTTGGATCTTTTG | | *KKFGSFGFLFFFXGLXGPKKPLX |
| | | GATTTCTTTTTTTTTTTTTNGGGCTTTNAGGNCCAAAAAAACCCCTA | | KXXPPXXGGFFFFFXXXPKXPXWG |
| | | AANAAAANNNNNCCCCCCAANTGNGGNGGNTTTTTTTTTTTTTTTNG | | FFPXPPLPXXGGXPPXXAPPLIXP |
| | | GNCCANCCCCAAAAANCCCNGGTGGGGNTTTTTCCCNTNCCCCCCCC | | PGGGXXXNXPKKXPGAXGGXXGKP |
| | | ThCCGNNTTTNGGNGGGTNCCCCCCCCNAANGGCCCCNCCCCTNATT | | XXFXXFGKTPXPKKXXXFXKXFF |
| | | TNNCCCCCGGGGGGGGGCNTTTTNNAAAACCNCCCCAAAAAAAANCC | | |
| | | GGGGGCCNAGGGGGGGGNNNNGGGGAAACCNTNTNCNTTTTTNNNTT | | |
| | | TTGGGAAAACNCCNNCCCCNAAAAAAANCCNNNTTTTTTGNAAAANN | | |
| | | TTTTTTT | | |
| prey78767 | 610 | ACGCAAGAACCTAAGCATTTGCTNGAGGGTACAATTATTGCTTCCAA | 611 | TQEPKHLLEGTIIASKIVLTFLGX |
| | | AATAGTCCTTACATTTTTAGGCTNTCTTTGAGAGGCTAAGTCTTGCC | | L*EAKSCLSFXSLSYSWLENSTEV |
| | | TCTCATTCANTTCACTTTCTTACTCCTGGCTTGAGAATTCTACTGAA | | *DSWPTSPTCLPKMKSIMSFCNRW |
| | | GTATGAGACAGCTGGCCAATAAGCCCAACTTGTTTGCCAAAGATGAA | | *A*VPIETY*AQWQCYNLIFPCQG |
| | | ATCCATTATGTCTTTTTGCAACAGATGGTGAGCGTAAGTCCCAATAG | | S*KKDMLNWINSKVYL**AKNYSC |
| | | AGACATACTGAGCTCAATGGCAGTGCTACAACTTGATTTTCCCTTCC | | SENPFLFSLNHEAEWDESKYRNSH |
| | | CAGGGGAGCTAGAAAAAGGACATGCTCAATTGGATCAATAGTAAAGT | | *NQPYPL*XEFXHVINHRYHKGG |
| | | TTACTTATAGTGAGCCAAGAATTACTCCTGTAGTGAGAACCCATTTC | | |
| | | TATTTTCATTAAACCATGAAGCTGAATGGGATGAATCTAAATATAGA | | |
| | | AATTCACATTAGAATCAACCATATCCTCTCTAACNTGAATTTNAACA | | |
| | | TGTTATTAACCATAGATATCACAAAGGAGGAC | | |
| prey32308 | 612 | ATGTACGAGCAACTCAAGGGCGAGTGGAACCGTAAAAGCCCCAATCT | 613 | MYEQLKGEWNRKSPNLSKCGEELG |
| | | TAGCAAGTGCGGGGAAGAGCTGGGTCGACTCAAGCTAGTTCTTCTGG | | RLKLVLLELNFLPTTGTKLTKQQL |
| | | AGCTCAACTTCTTGCCAACCACAGGGACCAAGCTGACCAAACAGCAG | | ILARDILEIGAQWSILRKDIPSFE |
| | | CTAATTCTGGCCCGTGACATACTGGAGATCGGGGCCCAATGGAGCAT | | RYMAQLKCYYFDYKEQLPESAYMH |
| | | CCTACGCAAGGACATCCCCTCCTTCGAGCGCTACATGGCCCAGCTCA | | QLLGLNLLFLLSQNRVAEFHTELE |
| | | AATGCTACTACTTTGATTACAAGGAGCAGCTCCCCGAGTCAGCCTAT | | RLPAKDIQTNVYIKHPVSLEQYLM |
| | | ATGCACCAGCTCTTGGGCCTCAACCTCCTCTTCCTGCTGTCCCAGAA | | EGSYNKVFLAKGNIPAESYTFFID |
| | | CCGGGTGGCTGAGTTCCACACGGAGTTGGAGCGGCTGCCTGCCAAGG | | ILLDTIRDEIAGCIEKAYEKILFT |
| | | ACATACAGACCAATGTCTACATCAAGCACCCAGTGTCCCTGGAGCAA | | EATRILFFNTPKEKMTDYAKKRGW |
| | | TACCTGATGGAGGGCAGCTACAACAAAGTGTTCCTGGCCAAGGGTAA | | VLGP |
| | | CATCCCCGCCGAGAGCTACACCTTCTTCATTGACATCCTGCTCGACA | | |
| | | CTATCAGGGATGAGATCGCTGGGTGCATCGAGAAGGCCTACGAGAAA | | |
| | | ATCCTTTTCACTGAGGCCACCCGGATCCTCTTCTTCAACACACCCAA | | |
| | | AAAGATGACAGACTACGCCAAGAAGCGAGGGTGGGTCCTGGGCCCC | | |
| prey78770 | 614 | GGGCTCGNTGNTTTAACTGGACAGAAAGAGACACCGTGCTGCTCACT | 615 | GLXXLTGQKETPCCSLLPSTGXXW |
| | | CCTGCCTTCCACAGGCNGNTNCTGGNCACTGGNGAGGGAGNGGACTC | | XLXREXTPXTXCGEEQXVLTILYG |
| | | CANAGACAANAGGAGGAGAGGAACAGCNNGTGCTTACTATCCTTTAC | | VXXKEEXXWAVAGQGSXNKXEXFL |
| | | GGAGTGNAGAANAAAGAGGAGGANANNTGGGCNGTGGCTGGACAGGG | | KVRAIVAWLQVGGWXLTEKXTWVL |
| | | GAGTTGNAACAAGAANGAANTCTTCTTAAAGGTCAGAGCAATAGTGG | | XRESXDLLXXXSLX*GDPXXXAGX |
| | | CATGGTTGCAGGTGGGAGGGTGGCANCTGACAGAGAAAANCACNTGG | | XXXXLCXX*RXPXXXV |
| | | GTGCTGNAAAGGGAGAGTGNGGACTTGCTGNANNAAATNTCNCTGGN | | |
| | | ATAGGGGGATCCNNGGCNACANGCTGGGNGANNNNNCNANCNTCTCT | | |
| | | GCAGNTNNTGACGCTNGCCCCNNANNNTNGTTT | | |
| prey78772 | 616 | GNGANGCAAGCTANGTTACNTTANGGNNGAAAAAAAGAGCAACCTGN | 617 | XXQAXLXXGXKKEQPXILQRDCXS |
| | | CATACTNCAAAGAGATTGTNTNAGTGTANAACNANNTTGNATNTNTT | | VXXXXXXXIXFFFXXPKXPXTXLK |
| | | GNATTNTTTTTTTTTTTNTNCNCCCTAAANGCCCAANGACCNNCTTA | | KRDXKXXXXFXFFXX |
| | | AAAAAGAGGGACCNNAAACNTAANGNGNCNTTTNTTTTTTTTANGGN | | |
| | | G | | |
| prey78773 | 618 | GCGGGGCAAGCTACGTTACATTATGGCAGAAAAAAAGTGCAACTGAC | 619 | AGQATLHYGRKKVQLTYYKEIF*V |
| | | ATACTACAAAGAGATTTTTTAAGTTTAAAAAAAGTTTGGATCTTTTG | | *KKFCSFGFFFFLGXXGLKNA*II |
| | | GATTTTTTTTTTTTTTGGGNNTTNATGGCCTAAAAAACCCCTAAATA | | ITPXNKEXFFFFFGQXPKNPVGVF |
| | | ATAATAACCCCCNCAAATAAGGAGGNTTTTTTTTTTTTTTTTGGNCA | | SLXPPSQXFRVPFPKEPPXIYPPG |
| | | AANCCCAAAAAACCCGGTGGGGGTTTTTTCCCTTNCCCCCCCTTCCC | | GPXVKPPKKNRAQRGKRGXLALLV |
| | | AANTTTTCCGGGTCCCATTCCCCAAGGAACCCCCCNTAATTTACCCC | | WKNNPPKKTPFFQKFX |
| | | CCAGGGGGGCCTTTNGTAAAACCCCCNAAAAAAAACCGGGCCCAAAG | | |
| | | GGGGAAAAGGGGAANCCTGGCCCTTTTGGTTTGGAAAAACAACCCCC | | |
| | | CCAAAAAAACCCCATTTTTTCAAAAATTTTNTT | | |
| prey323 | 620 | ATTCGTTCCTGCATGACTCCCAGACCTCCTTCTGCTTCTCAGACTCT | 621 | SFLHDSQTSFCFSDSIPTPSNMEE |
| | | ATTCCGACACCCTCCAACATGGAGGAAACGCAACAGAAATCCAATCT | | TQQKSNLELLRISLLLIESWLEPV |
| | | AGAGCTGCTCCGCATCTCCCTGCTGCTCATCGAGTCGTGGCTGGAGC | | RFLRSMFANNLVYDTSDSDDYHLL |
| | | CCGTGCGGTTCCTCAGGAGTATGTTCGCCAACAACCTGGTGTATGAC | | KDLEEGIQTLMGRLEDGSRRTGQI |
| | | ACCTCGGACAGCGATGACTATCACCTCCTAAAGCACCTAGAGGAAGG | | LKQTYSKFDTNSHNHDALLKNYGL |
| | | CATCCAAACGCTGATGGGGAGGCTGGAAGACGGCAGCCGCCGGACTG | | LYCFRKDMDKVETFLRMVQCRSVE |
| | | GGCAGATCCTCAAGCAGACCTACAGCAAGTTTGACACAAACTCGCAC | | ACSCGF* |
| | | AACCATGACGCACTGCTCAAGAACTACGGGCTGCTCTACTGCTTCAG | | |
| | | GAACGACATGGACAAGGTCGAGACATTCCTGCGCATGGTGCAGTGCC | | |
| | | GCTCTGTGGAGGGCAGCTGTGGCTTCTAG | | |
| prey78778 | 622 | ATTCCAATGCATCCCTGCTGATCCAGAATGTCACCCGGGAACACGCA | 623 | SNASLLIQNVTREADAGSYTLHII |
| | | GGATCCTACACCTTACACATCATAAAGCGAGGTGATAGGACTAGAGG | | KRGDRTRGVTGYFTFNLYLKLPKP |
| | | AGTAACTGGATATTTCACCTTCAACTTATACCTGAAGCTGCCCAAGC | | YITIKNSKPRENKDVLAFTCEPKS |
| | | CCTACATCACCATCAAGAACTCAAAACCCAGGGAGAATAAGGATGTC | | ENYTYIWWLNGQSLPVSPRVKRPI |
| | | TTAGCCTTCACCTGTGAACCTAAGAGTGAGAACTACACCTACATTTG | | ENRILILPSVTRNETGPYECEIRD |
| | | GTGGCTAAATGGTCAGAGCCTCCCGGTCAGTCCCAGGGTAAAGCGAC | | RDGGMHSDPVSLNVLYGPDLPSIY |
| | | CCATTGAAAACAGGATCCTCATTCTACCCAGTGTCACGAGAAATGAA | | PSFTYYRSGENLYLSCFAESNPPA |
| | | ACAGGACCCTATGAATGTGAAATACGGGACCGAGATGGTGGCATGCA | | EYFWTINGKFQQSGQKLSIPQITT |
| | | CAGTGACCCAGTCTCCCTGAATGTCCTCTATGGTCCAGACCTCCCCA | | KHRGLYTCSVRNSATGKESSKSMT |
| | | GCATTTACCCTTCATTCACCTATTACCGTTCAGGAGAAAACCTCTAC | | VEVSAPSGIGRLPLLNPI* |
| | | TTGTCCTGCTTCGCGGAATCTAACCCACCGGCAGAGTATTTTTGGAC | | |
| | | AATTAATGGGAAGTTTCAGCAATCAGGACAAAAGCTCTCTATCCCCC | | |
| | | AAATTACTACAAAGCATAGAGGGCTCTATACTTGCTCTGTTCGTAAC | | |
| | | TCAGCCACTGGCAAGGAAAGCTCCAAATCCATGACAGTCGAAGTCTC | | |
| | | TGCTCCTTCAGGAATAGGACGTCTTCCTCTCCTTAATCCAATATAG | | |
| prey78782 | 624 | CTGGCCTTTTCACTTTTTAAGTTTTATGATAACATATTTTAATTTTG | 625 | LAFSLFKFYDNIF*F*RSPVSLFC |
| | | AAGAAGTCCAGTTTCTCTCTTTTGTTTTACATTTAGTACTTTTTGTG | | FTFSTFCVVSKNVCLDQGCECFLL |
| | | TGGTCTCCAAAAATGTTTGCCTAGACCAAGGTTGTGAATGTTTTCTC | | YFVLEVL*L*HLCLGL*TTMN*FS |
| | | CTATATTTTGTTCTAGAAGTTTTATAACTTTAGCATTTATGTTTAGG | | WAGKGPKFFFF*PLSXCSSITC*X |
| | | GTTATGAACCACTATGAATTAATTTTCTTCGGCAGGGAAGGGGCCAA | | TLLTKFCFIKYLFY*XVIFVSM*Y |
| | | AGTTCTTTTTTTTTTAACCTTTATCCANCTGTTCCAGCATTACTTGT | | FY*NFSVFIXXXGXR |
| | | TGANAAACTCTTCTCACTAAATTTTGTTTTATAAAATATCTTTTTTA | | |
| | | TTGAAANGTTATTTTTGTAAGCATGTAATATTTTTATTAAAATTTCT | | |
| | | CAGTTTTCATTCNGAANANGGGNAANAGGG | | |
| prey78783 | 626 | GCATACTCCTGCATGTGCAAGGGGATTTGGCCTTGCTACCTTTATGG | 627 | AYSCMCKGIWGCYLYGFLALRGRL |
| | | GTTTCTTTAACTGAGGGGTAGATTATTCCTGACAAAAGGTGAAGATT | | FLTKGEDFSELCYHLFLHQWVFSE |
| | | TCTCAGAACTGTGTTACCACCTATTTTTACACCAAATATGGGTGTTC | | VSWCWWGV*FSMLMSV*CGPS*NL |
| | | TCGGAAGTGTCCTGGTGCTGGTGGGGTGTGTGATTTAGTATGTTAAT | | GQIQFHVGFSWS*SALSILCFSRS |
| | | GAGCGTATAATGTGGTCCTAGTTGAAACCTAGGTCAAATCCAGTTCC | | *PMASAAISTVSFC*SCEIAAQNL |
| | | ATGTTGGGTTTAGTTGGTCTTAGTCAGCTTTGTCTATACTCTGTTTT | | LLFXXHLILFLSXDLR |
| | | TCACGGTCTTAGCCCATGGCCTCTGCAGCCATTTCAACAGTTTCCTT | | |
| | | TTGCTAGTCATGTGAAATTGCTGCCCAAAATCTTCTATTATTCTNCA | | |
| | | NCCACCTTATATTATTCCTGTCTCANGACCTAAGG | | |
| prey78785 | 628 | TTGTCTTCATGCCATGTCATTTTATACAGTACTTTGTAGGTTTTGAT | 629 | LSSCHVILYSTL*VLMRKLVFLMH |
| | | GAGAAAGTTGGTATTTTTAATGCACTCAAAAATTGCTTTTTATTGGA | | SKIAFYWRVLINITF*SVN*DKRH |
| | | GAGTACTTATCAATATAACTTTCTGATCAGTTAATTAAGATAAAAGG | | TLSTGERIKSVSIIFFCL*KEKLI |
| | | CATACATTGTCCACAGGTGAGAGGATTAAATCAGTTAGCATTATTTT | | KFLNGVIKQSFTLIAQAGVQIYCG |
| | | TTTCTGTTTGTAAAAGGAAAAACTTATTAAATTTTTAAACGGAGTTA | | KYKIC*KIHLRN*VIREQMVSGEF |
| | | TTAAACAGAGTTTCACTCTTATTGCCCAGGCTGGAGTGCAAATATAT | | *I*FSMI*HNIH |
| | | TGTGGCAAATATAAAATTTGCTAGAAAATACATCTTAGAAATTAAGT | | |
| | | TATAAGAGAGCAAATGGTAAGTGGAGAATTTTAAATTTAATTTTCTA | | |
| | | TGATATAGCATAACATACAT | | |
| prey78790 | 630 | ATTACCCTCTATGGAGGGCTCCTGTGAAGATTAATGAATACTTTTTA | 631 | ITLYGGLL*RLMNTF*SFRALIVA |
| | | GTCATTTAGGGCATTAATTGTGGCACAGTGAGTGCACAATAATTGCA | | QAVHNNCIIHRLYTNKYHCIYNNN |
| | | TAATACATAGGTTATATACNAATAAATATCATTGTATTTATAATAAT | | KVSAFFGNFSLLILYSVFLRFFFL |
| | | AATAAAGTGTCAGCTTTTTTTGGTAACTTCAGCCTTCTCATTCTCTA | | FTFVSPPTWKMLSICAIFQCCTTI |
| | | TTCTGTGTTTCTTAGATTTTTTTTTCTTTTCACTTTTGTCTCTCCTC | | SSFSNS**SLLLNSHCI*LYITLQ |
| | | CCACCTGGAAAATGCTCTCAATCTGTGCCATCTTTCAATGCTGTACC | | HLLLFHICSFFCC*T*AS*GRCDF |
| | | ACAATCTCCAGTTTCTCTAACTCCTAATGATCTCTTCTTCTAAACAG | | KCVLIFIRYPST*ENRLQPKCSRL |
| | | CCATTGCATTTAATTGTATATTACCTTACAACATTTGCTGCTATTTC | | KIN*NKGNKKQVQCH*AKPL |
| | | ACATATGCAGCTTTTTCTGCTGTTAGACATAAGCATCTTGAGGACGG | | |
| | | TGTGACTTCAAGTGTGTACTAATTTTTATAAGGTATCCTTCTACCTA | | |
| | | GGAAAACAGGCTCCAGCCTAAATGCTCCAGGCTTAAAATAAATTAGA | | |
| | | ATAAGGGTAATAAAAAGCAGGTACAGTGCCATTAAGCCAAACCTCTC | | |
| | | C | | |
| prey78486 | 632 | CTCCAGTGCTATGGGAGCATCGGCTACTGCTGGTGTGTCTTCCCCAA | 633 | LQCYGSIGYCWCVFPNGTEVPNTR |
| | | CGGCACGCAGGTCCCCAACACCAGAAGCCGCGCGCACCATAACTGCA | | SRGHHNCSESLELEDPSSGLGVTK |
| | | GTGAGTCACTGGAACTGGACGACCCGTCTTCTGGGCTGGGTGTGACC | | QDLCPVPM* |
| | | AAGCAGGATCTGGGCCCAGTCCCCATGTGA | | |
| prey78791 | 634 | TCCCACCTCAGCCCCCTGAGCAGCTGCAACTACAGGCATGAGCCACC | 635 | SHLSPLSSCNYRHEPPCSXXXXXX |
| | | CTGTTCCANNNNNNNNNNNNNNNNNNNNNNNNNNNNNNNNNNNNNNN | | XXXXXXXXXXXXXXXXXXXXXXXX |
| | | NNNNNNNNNNNNNNNNNNNNNNNNNNNNNNNNNNNNNNNNNNNNNNN | | XXXXXXXXXRPGQQQTLYMHKHNK |
| | | NNNNNNNNNNNNNNNNNNNNNNNNNNNNTCAGGCCTGGCCAACAACA | | YVKKERRETYCVYPML*INAIQLQ |
| | | AACTCTTTACATGCATAAACATAATAAATATGTTAAAAAAGAGAGGA | | QKDFKLGITQLPGLSKDFKLGRVM |
| | | GAGAAACCTACTGTGTGTACCCCATGCTCTAGATAAATGCCATACAG | | QSRR |
| | | CTACAACAAAAAGACTTTAAATTGGGGATAACCCAGCTGCCTGGGTT | | |
| | | ATCAAAAGATTTTAAGTTGGGGAGGGTAATGCAGTCTCGAAGGC | | |
| prey78798 | 636 | NNNNNNNNNNNNNNNNNNNNNNNNNNNNNNNNNNNNNNNNNNNNNNN | 637 | XXXXXXXXXXXXXXXXXXXSFY*P |
| | | NNNNNNNNNNTCTTTTTATTAACCTCTAATACCTAAAACCTACTACA | | LIPKTYYNXGSIAIARSGLEDL*V |
| | | ATTNCGGGAGCATAGCAATCGCTAGATCTGGNCTGGAAGATCTCTGA | | SN*LFNKMLCFVPCXGTRT*LYEP |
| | | GTATCTAACTAGCTTTTTAATAAAATGCTTTGTTTTGTACCATGTNG | | LMN*KKKXGPPXXXXKXXGXVXXK |
| | | AGGTACCAGGACTTGACTTTATGAGCCTCTTATGAATTAAAAAAAA7 | | KKKKKRXXFXKXKXXXXXXGGGGF |
| | | AAANGGNCCNCCCNGTTTNNGNNNGAAAANANNGGGTTTNGTNNTTN | | PPPFXFFFFX |
| | | GCAAAAAAAAAAAAAAAAAAGANGGTTNTTTNTGAAAANAAAAAANN | | |
| | | ANANANGTNCNNNNGGGGGGGGGGGNTTCCCCCCCCCNTTTNNTTTT | | |
| | | TTTTTTTTTNGGNC | | |
| prey78807 | 638 | TCGGGGCAAGCTACGTTACATTATGGCAGAAAAAAAGTGCAACTGAC | 639 | SGQATLHYGRKKVQLTYYKEIF*V |
| | | ATACTACAAAGAGATTTTTTAAGTTTAAAAAAAGTTTGGATCTTTTG | | *KKFGSFGFFFFXXFXALKXR*XX |
| | | GATTTTTTTTTTTTTTNGNNTTTTANGCNCTAAAAANCCGCTAANTT | | XTPPXXGXFFFFXGLPXQKXXXGX |
| | | NTANTNACCCCCCCAAANAANGGAGANTTTTTTTTTTTTTTNGGGCT | | FSXXXXXXFGXXPXPXGXTXXXXP |
| | | CCCCCNCCAAAAANCCNTNTNNGGGNTTTTTTCNTTNCNCNCCNTTN | | XGGAXWXXPPXPNPGXXGGXRGXA |
| | | GTNGTTTTGGNGNNNCNCCCNNCCCCNANGGGNNTACCCNTTNNNTC | | XPXXXXGXXRPPPXXPXFLX |
| | | NCCCCCNNGGGGGGGGCCNTNTGGNGGANCCCACCTNNCCCAAACCC | | |
| | | CGGNCNCNNNGGGGGGGNCCGGGGGNNCGCCNCGCCNCNNNNTNGNN | | |
| | | CNGGANNNNCCCGGCCCCCCCCCNGNNCCCCCNCTTTTTTGNNG | | |
| prey78811 | 640 | ATGCACCTGTGCACACTCACATCCACCCATACACACACACACACACA | 641 | MHLCTLTSTHTHTHTHTHTHTHTP |
| | | CACACACACACACACACACACTCCCTTACACTGTTGTACTTCCGTGG | | LHCCTSVGPPSRTRTTRTRLKGLL |
| | | GCCCTCCTTCCAGAACAAGGACAACAAGGACAAGGTTGAAGGGTCTT | | IYHGLLPSMCLAQ* |
| | | CTCATCTACCATGGCCTGCTACCTAGCATGTGTCTAGCTCAATGA | | |
| prey78823 | 642 | CGAGGAGGCCGTAGGGAAGGCGGGGAGTGGTCCTAGCACCACAGGGT | 643 | EEAVGKAGSGPSTTGLGAEGAEGV |
| | | TGGGAGCAGAGGGAGCGGAGGGCGTTTGCCCCTGCCCCGGGCATCTC | | CPCPGHLCPHSLWGPPAVPLRRPG |
| | | TGCCCTCATTCGCTCTGGGGCCCCCCGGCACTGCCACTAAGGAGGCC | | PVQSQISTQCWVFLLWL*TGCFLS |
| | | AGCCCCTGTCCAGAGTCAAATCTCCACTCAGTGTTGGGTGTTTCTCC | | KHLIYSLRKAATGAN |
| | | TCTGGCTGTAAACAGGGTGTTTCTTAAGTAAACATTTAATATACTCC | | |
| | | CTCAGAAAGGCAGCCACAGGGGCAAAC | | |
| prey78824 | 644 | CCGGATGCTGGCTTTTGTGTGTGTGTGAATGAGTGTTATTTCCTTTA | 645 | PDAGFCVCVNECYFLYCFI*MGLY |
| | | TTGTTTTATATAGATGGGATTATACTAATTGTTATATCCTAACAATT | | *LLYPNN**LYTDCINVILTFI*M |
| | | AATAGTTATATACTGACTGTATAAATGTTATACTCACATTTATATAG | | GIYYSFFVVTYHGLLSQPVLSASV |
| | | ATGGGAATATACTATTCCTTTTTTGTTGTTACTTATCATGGCCTCCT | | SEVRVGFTVSWGTEVL*VVEPSCL |
| | | CTCCCAGCCTGTTCTGTCTGCCTCGGTCTCTGAAGTCCGTGTAGGAT | | DAYF*SFPDAYIIQ*NSFCVDLLM |
| | | TCACAGTATCATGGGGGACAGAAGTGCTATAGGTTGTTGAACCCTCC | | YLYMQIFSKYS*N*IPRIAGLVGF |
| | | TGTCTTGACGCATATTTCTAGTCATTTCCAGATGCCTACATAATCCA | | LILIS*NRSLEKFWYLSLDQRHQH |
| | | GTAAAACTCCTTCTGTGTAGATCTTCTGATGTACTTGTATATGCAGA | | F*MKLELICVLESISN |
| | | TTTTTAGCAAATATTCCTAAAATTGAATTCCTAGAATTGCTGGGCTG | | |
| | | GTAGGGTTTTTAATTCTGATATCGTGAAATCGATCGCTAGAGAAATT | | |
| | | TTGGTACCTCTCTTTGGATCAAAGGCATCAGCATTTTTAAATGAAGC | | |
| | | TTGAACTGATTTGTGTACTGGAATCCATATCAAACT | | |
| prey78825 | 646 | ACTCTACAATTCTCCCAGTGATCGCACTAAGTCGCCAAAGTTCCCTT | 647 | LYNSPSDRTKSPKFPYTRRRNPSC |
| | | ACACGCGTCGCCGAAACCCCTCCTGTGGAAGTGACAATGATTCTGTA | | GSDNDSVQPVRRRKAHNSGEDSDL |
| | | CAGCCTGTGAGGAGGAGGAAAGCCCATAACAGTGGTGAAGATTCAGA | | KQRRRSRSRCNTSSGSESENSNRE |
| | | TCTTAAGCAAAGGAGGAGGTCACGTTCACGCTGTAACACCAGCAGTG | | HRKKRNRIRQENDMVDSAPQWEAV |
| | | GTAGTGAATCAGAAAATTCTAATAGAGAACACCGGAAAAAGAGAAAC | | LRRQKEKNQADPNSRRSRHRSRSR |
| | | AGAATACGGCAGGAGAATGATATGGTTGATTCAGCGCCTCAGTGGGA | | SPDIQAKEELWKHIQKELVDPSGL |
| | | AGCTGTATTAAGGAGACAAAAGGAAAAAAACCAAGCCGACCCCAACA | | SEEQLKEIPYTKTETQGDPIRIT |
| | | GCAGGCGATCCAGACACAGATCTCGTTCGAGAAGCCCCGATATCCAA | | |
| | | GCAAAAGAAGAGTTATGGAAGCACATTCAAAAAGAACTTGTGGATCC | | |
| | | ATCCGGATTGTCCGAAGAACAATTAAAAGAGATTCCATACACTAAAA | | |
| | | TAGAGACACAAGGTGACCCAATCCGCATCACGC | | |
| prey78828 | 648 | TNCCATNCTGGTCNATGTTGTGAATGGACTTTACTGGGCACATTCTG | 649 | XHXGXCCEWTLLGTF*DPAGK*YI |
| | | AGATCCTGCCGGAAAGTAATATATATACAGTTGGCCCTCTGTATCTA | | YSWPSVSMDSTNLESKVFRKTIKM |
| | | TGGATTCAACCAATCTTGAATCAAAAGTATTTAGGAAAACAATTAAA | | I*SILGGGA*AICKYHIILYKGLE |
| | | ATGATTTAAAGTATATTGGGAGGAGGTGCATAGGCCATATGCAAATA | | HRWISVSKGSPGNNPLQILRDDLH |
| | | CCACATTATTTTATATAAGGGACTTGAGCATCGCTGGATTTCTGTAT | | TKNEP*FFLFCWAQNCSWS*KI*L |
| | | CCAAGGGGAGTCCTGGAAACAATCCCCTGCAGATACTGAGGGACGAC | | LWLRTIHGRRW*TL |
| | | CTGCATACAAAAAATGAACCATAATGATTCTTCTTGTTTTGCTGGGC | | |
| | | CCAGAATTGCTCTTGGTCATAGAAAATTTAACTTCTTTGGCTAAGAA | | |
| | | CAATCCACGGTAGAAGATGGTGAACCTTG | | |
| prey78830 | 650 | GGGGCAAGCTACGTTACATTATGGCAGAAAAAAAGTGCAACTGACAT | 651 | GQATLHYGRKKVQLTYYKEIF*V* |
| | | ACTACAAAGAGATTTTTTAAGTTTAAAAAAAGTTTGGATCTTTTGGA | | KKFGSFGFLFFFLGFKGLKNP*LK |
| | | TTTCTTTTTTTTTTTTTGGGCTTTAAGGGCCTAAAAAACCCCTAATT | | LPPPKRXVFFFFLGPPQKXXVXVF |
| | | AAAATTACCCCCCCCAAAAAGGGANGTTTTTTTTTTTTTTTTGCGCC | | SFXPLSXFXXVPFPKXXPXXSPQG |
| | | CCCCCCAAAAAANCCNNGTANGGGTTTTTTCCTTTNNCCCCCTTTCC | | GPXVKPPKKNRXERGXRGTLPLFX |
| | | NGNTTTTGNGNGGTCCCATTCCCAAAGGNGNCCCCCTTNNTTTCCCC | | XKKRAPPKTPFFXNFF |
| | | CCAAGGGGGCCCCTTNGTNAAACCACCAAAAAAAAACCGGNTNGAAA | | |
| | | GGGGGNCNAGGGGAACCCTTCCCCTTTTTNTTTGNAAAAAAAGGGCC | | |
| | | CCCCCCAAAACCCCCTTTTTTTGNAACTTTTTT | | |
| prey78838 | 652 | GCGNTTCCNTATAAATTATATTACTTTTTCTTTCTATTTCTGGGAAA | 653 | AXPYKLYYFFFLFLGKRSXVFXLG |
| | | AACATCANTGGTATTTNGATTGGGATTGCATTGAANCTNTNGACGGN | | LH*XXXRXGVARTFXQY*FFFXSX |
| | | GNGGGGTAGCAAGAACATTTNAACAATATTAATTTTTTTTTCANTCC | | XMEYFSXFSXTLHFFSSVFYSXQY |
| | | ANGCANATGGAATATTTTTCCNTTTTTTCANGTACTCTTCATTTTTT | | RLLSXL*FXGX*FYIXLR*IKFAK |
| | | TTCATCAGTGTTTTATAGTTNTCAGTATAGACTTCTTTCANTTCTTT | | XFSHGSLXIYRTATYFCMSIFASS |
| | | AATTCCNAGGTNTTTAGTTTTATATCTNACTACGGTAAATAAAATTT | | QLYXICLSXLXV |
| | | TAAAAANTTTTTTCACATGGTTCACTGTTNATATATAGAACTGCCAC | | |
| | | ATATTTTTGTATGTCGATTTTTGCATCATCGCAACTTTACNGAATTT | | |
| | | GTTTATCANTTCTNANAGTN | | |
| prey78842 | 654 | GCNGGTGTGNTTTTTTCTNGTTTTGGNGGACTTCATCATCACATNCT | 655 | AGVXFSXFGGLHHHXL*IFXRYRG |
| | | ATGAATTTTTGNGAGATATAGAGGCANAATATNCTGNNTTGCCATTC | | XIXXXAILHSXLGTGQW*SFIAVF |
| | | TACATAGCANTTTAGGGACTGGGCAGTGGTAAAGTTTTATTGCAGTT | | CAAE*D*NFSX**CPQLXRXXLNS |
| | | TTTTGCGCTGCGGAATGAGATTGAAATTTTTCTGANTAATAATGCCC | | FESWLXLXISXYFXTNSP*NLQGX |
| | | TCAACTATNAAGGNAANTTACTTAATAGCTTTGAAAGTTGGCTTTTN | | TVIIXELLYCGNGXXLXEXMNHXX |
| | | CTGNANATTTCATANTATTTTGNNACAAATTCACCCTAAAATTTACA | | XEXXTNRXKXVXPXXFYTPX |
| | | AGGCANAACAGTGATTATAAGNGAACTCTTATACTGTGGNAACGGNC | | |
| | | NCTNTCTNCNAGAATNAATGAATCATGANCNNCANGAGNACANAACA | | |
| | | AATAGGGNGAAAANGGTNTTNCCAATNNCCTTTTACACCCCTTNAT | | |
| prey78843 | 656 | TCAGATACAAAATCNAATACATTTTTATATGGCAGCAATAAACAGACT | 657 | SIDTKSNTFLYGSNKQTIYTIYPF |
| | | ATATACACTATATACCCATTTATTTAATAATCACAACAAAAATACAA | | IE**SQQKYKEPRSKFFKKTYKTF |
| | | AGAACCTCGGAGTAAATTTTTTAAAAAGACTTATAAGACCTTTATTT | | I*KTI*TL*NTSIIVKRYHVQRYY |
| | | AGAAAACAATTTAAACTTTGTAAAATACCTCAATAATAGTAAAGAGG | | KTMNYLQIYPQIQCNLK*NLSXXX |
| | | TATCATGTTCAGAGGTATTATAAAACTATGAATTATCTTCAAATTTA | | XXXXXXXXXXXXXXXXXXXXXXXX |
| | | TCCACAGATTCAATGCAATCTCAAATAAAATTTATCNNNNNNNNNNN | | XXXXXXXXXXXXXTSAV |
| | | NNNNNNNNNNNNNNNNNNNNNNNNNNNNNNNNNNNNNNNNNNNNNNN | | |
| | | NNNNNNNNNNNNNNNNNNNNNNNNNNNNNNNNNNNNNNNNNNNNNNN | | |
| | | NNNNNNNNNNNNNNNCACCTCCTGAGTG | | |
| prey78846 | 658 | GCTGATTGTGGAGGTCGAANGGTTGAANACTTGAGAAATAGCCNGAG | 659 | ADCGGRXVEXLRNSXRVSXMMGET |
| | | GGTATCTNACATGATGGGGGAGACAATGGGGAGCCTTTGGAGGTTCT | | MGSLWRFYGRV*S*LKHXLVLTGL |
| | | ATGGCAGGGTGTGATCATGATTAAAACACANCTTAGTCCTTACAGGA | | XXQVKEQQQEMEQTEXXLFLPSSX |
| | | CTANGTANGCAGGTNAAGGAACAGCAACAGGAGATGGAACAGACAGA | | RNSXFXLXDCRNVSFSPFTYFPFL |
| | | ANACNACCTCTTCCTTCCCAGCTCCNGCAGGAATTCTNACTTCANCC | | SFCDILSRLKSWGRESLKGVIWER |
| | | TCANGGACTGCAGAAATGTCTCATTTTCCCCGTTCACTTATTTTCCA | | LTQ*LWSNRGGQG*GS*AGECVPR |
| | | TTTCTTTCATTTTGTGACATTCTGAGTCGTCTTAAATCTTGGGGGAG | | EEAPQVGHTP*GKGWCSLGSGGSS |
| | | GGAAAGCCTGAAGGGGGTAATCTGGGAGAGGCTCACTCAGTAACTGT | | SFWPLPHLPATFPSCFLPTPAVHH |
| | | GGTCCAACAGGGGTGGGCAAGGATAAGGAAGTTAAGCTGGGGAGTGC | | |
| | | GTTCCCAGGGAAGAAGCCCCCCAGGTGGGGCACACACCCTAGGGGAA | | |
| | | GCGCTGGTGCAGCCTGGGGTCTGGTGGCAGCTCCTCCTTCTGGCCCC | | |
| | | TGCCCCACCTTCCAGCCACATTTCCTAGCTGCTTTCTGCCCACACCC | | |
| | | GCTGTGCACCAC | | |
| prey51748 | 660 | ATGGGGGAGAAACCCGGGACCAGGGTCTTCAAGAAGTCGAGCCCTAA | 661 | MGEKPGTRVFKKSSPNCKLTVYLG |
| | | CTGCAAGCTCACCGTGTACTTGGGCAAGCGGGACTTCGTAGATCACC | | KRDFVDHLDKVDPVDGVVLVDPDY |
| | | TGGACAAAGTGGACCCTGTAGATGGCGTGGTGCTTGTGGACCCTGAC | | LKDRKVFVTLTCAFRYGREDLDVL |
| | | TACCTGAAGGACCGCAAAGTGTTTGTGACCCTCACCTGCCCCTTCCG | | GLSFRKDLFIATYQAFPPVPNPPR |
| | | CTATGGCCGTGAAGACCTGGATGTGCTGGGCTTGTCCTTCCGCAAAG | | PPTRLQDRLLRKLGQHAHPFFFTI |
| | | ACCTGTTCATCGCCACCTACCAGGCCTTCCCCCCGGTGCCCAACCCA | | PQNLPCSVTLQPGPEDTGKACGVD |
| | | CCCCGGCCCCCCACCCGCCTGCAGGACCGGCTGCTGAGGAAGCTGGG | | FEIRAFCAKSLEEKSHKRNSVRLV |
| | | CCAGCATGCCCACCCCTTCTTCTTCACCATACCCCAGAATCTTCCAT | | IRKVQFAPEKPGPQ |
| | | GCTCCGTCACACTGCAGCCAGGCCCAGAGGATACAGGAAAGGCCTGC | | |
| | | GGCGTAGACTTTGAGATTCGAGCCTTCTGTGCTAAATCACTAGAAGA | | |
| | | GAAAAGCCACAAAAGGAACTCTGTGCGGCTGGTGATCCGAAAGGTGC | | |
| | | ACTTCGCCCCGGAGAAACCCGGCCCCCAGCC | | |
| prey82357 | 662 | GGNGTTTTTTTTNTTNTTGGTCTTGGGGNCCACCACNGCGCCNNGGA | 663 | GVFFXXGLGXHHXAXXLXCLTXXG |
| | | NCTNCNNTGCCTGACGANANANGGCAANGCAACTANGAAATCCTTGN | | XATXKSLXTVXLYAXTYIXXXKXD |
| | | NAACCGTCTNTCTCTATGCAANCACATACATATNCAGNGNGAAAGNG | | C*INLXDSXAQXVXXHWVXADLYC |
| | | GATTGTTAAATAAATCTANTTGATTCTGNNGCNCAAGNTGTTNACAT | | LXNSGPEXXRGR |
| | | NCACTGGGTCCNTGCNGACCTATACTGTCTTNTAAATTCTGGCCCCG | | |
| | | AANATANACGAGGGAGA | | |
| prey45630 | 664 | GATCTTCCGGCCCAAACACACCCGCATCTCCGAGCTGAAGGCTGAAG | 665 | IFRPKHTRTSELKAEAVKKDRRKK |
| | | CAGTGAAGAAGCACCGCAGAAAGAAGCTGACCCAGTCCAAGTTTGTC | | LTQSKFVGGAENTAHPRIISAPEM |
| | | GGGGGAGCCGAGAACACTGCCCACCCCCGGATCATCTCTGCACCTGA | | RQESEQGPCRRHMEASLQELKASP |
| | | GATGAGACAGGAGTCTGAGCAGGGCCCCTGCCGCAGACACATGGAGG | | RMVPRAVYLPNCDRKGFYKRKQCK |
| | | CTTCCCTGCAGGAGCTCAAAGCCAGCCCACGCATGCTGCCCCGTGCT | | PSRGRKRGICWCVDKYGMKLPGME |
| | | GTGTACCTGCCCAATTGTGACCGCAAAGGATTCTACAAGAGAAAGCA | | YVDGDFQCHTFDSSNVE* |
| | | GTGCAAACCTTCCCGTGGCCGCAAGCGTGGCATCTGCTGGTGCGTGG | | |
| | | ACAACTACGGGATGAAGCTGCCAGGCATGGAGTACGTTGACGGGGAC | | |
| | | TTTCAGTGCCACACCTTCGACAGCAGCAACGTTGAGTGA | | |
| prey82359 | 666 | TTTACAGTATATTTGTTATAAATATAATACATTTTTGAAAAGCTTAT | 667 | FTVYLL*I*YIFEKLIFN*LEIYI |
| | | TTTTAATTAACTTGAGATTTATATTTTAAACAGATCAACTCAATAGT | | LNRSTQ*LTLYNKEYGNFNDKNRI |
| | | TAACGTTATATAATAAAGAATATGGTAATTTTAATGACAAAAATCGT | | VK*RTLY*DE*GEKSQNSFQNYIQ |
| | | ATTGTGAAATAGCGCACTCTATACTGAGATGAATGAGGGGAAAAAAG | | SIRKNFFFFLQSYV*VIGTTKY*N |
| | | TCAAAACTCCTTTCAAAACTATATTCAAAGCATCAGAAAAAATTTTT | | MKTL*PKFKTDFKELCEARQENLY |
| | | TTTTCTTTTTACAAAGTTATGTATAAGTCATAGGGACCACCAAATAC | | LTLYRTSQ*SWNLETNG |
| | | TGAAATATGAAGACTCTATGACCAAAGTTCAAAACTGATTTTAAGGA | | |
| | | ACTGTGTGAAGCAAGACAGGAAAATTTGTATTTAACACTCTATAGAA | | |
| | | CTTCACAGTAAAGCTGGAATTTAGAGACTAATGGCT | | |
| prey77557 | 668 | GACCGACGAGAGCACCCGTAATGACGAGAGCTCGGAGCAAGAGAACA | 669 | TDESTRNDESSEQENNGDDATASS |
| | | ATGGCGACGACGCCACCGCATCCTCCAACCCGCTGGCGGGGCAGAGG | | NPLAGQRKLTCSQDTLGSGDLPFS |
| | | AAGCTCACCTGCAGCCAGGACACCTTGGGCAGCGGCGACCTGCCCTT | | NESFISADCTDADYLGIPVDECER |
| | | CAGCAACGAGTCTTTCATTTCGGCCGACTGCACGGACGCCGACTACC | | FREALLEIKCQVKSATPYGLYYPS |
| | | TGGGGATCCCGGTGGACGAGTGCCAGCGCTTCCGCGAGCTCCTGGAG | | GPLDAGKSDPESVDKELELLNEEL |
| | | CTCAAGTGCCAGGTGAAGAGCGCCACCCCTTACGGCCTGTACTACCC | | RSIELECLSIVRAHKMQQLKEQYR |
| | | TAGCGGCCCCCTGGACGCCGGCAAGAGTGACCCTGAGAGCGTGGACA | | flASWMLHNSGFRNYNTSTDVPAR |
| | | AGGAGCTGGAGCTGCTGAACGAAGAGCTGCGCAGCATCGAGCTGGAG | | HELSDTTELPEKSDKDNARAPTTQ |
| | | TGCCTGAGCATCGTGCGCGCCCACAAGATGCAGCAGCTCAAGGAGCA | | ARAAAAPRSPWRSPPTTP* |
| | | GTACCGCGAGTCCTGGATGCTGCACAACAGCGGCTTCCGCAACTACA | | |
| | | ACACCAGCATCGACGTGCGCAGACACGAGCTCTCAGATATCACCGAG | | |
| | | CTCCCGGAGAAATCCGACAAGGACAACGCTCGAGCGCCTACAACACA | | |
| | | GGCGAGAGCTGCCGCAGCACCCCGCTCACCCTGGAGATCTCCCCCGA | | |
| | | CAACTCCTTGA | | |
| prey82363 | 670 | ANNACTTTTACTTGNCCTTGGNTNNNTATANGGGTACTCGGNCCCCN | 671 | XTFTXPWXXIXVLGPXPTL*LGXT |
| | | ACCGACATTGTGATTAGGAAANACTTTANCAATAGGAAAACCCTCAT | | LXIGKPSXXSMXXQTXTCAXGVGI |
| | | ANCNATCTATGTNTANGCAAACACANACATGTGCANGGGGAGTGGGG | | XR*IXRXCLXKXEHFWX*XXVXLX |
| | | ATAGNNAGATAAATANATAGAANTTGCTTANNNAAANGAGAACATTT | | KXXXXXXIFXXXXLXXSWXCTXTQ |
| | | CTGGNTNTAANAAANNGTTTNGCTTTNAAAANCNANCGNGAANNTTN | | GNGXXIXKVLXICDFVXXXLXXTP |
| | | NGATCTTTNAANNCNTTCNCCTTNTTTNTTCCTGGNACTGCACTCNA | | TXKXRXIXI |
| | | ACACAAGGGAATGGGANTNNTATTNTGAAGGTGTTGTGNATCTGCGA | | |
| | | CTTTGTGTNNNTGGNACTAAANNCTACTCCAACTTNCAAATGNAGAN | | |
| | | TGATTTGNATTT | | |
| prey32308 | 672 | CTTCTTGCCAACCACAGGGACCAAGCTGACCAAACAGCAGCTAATTC | 673 | FLPTTGTKLTKQQLILARDILEIG |
| | | TGGCCCGTGACATACTGGAGATCGGGGCCCAATGGAGCATCCTACGC | | AQWSILRKDIPSFERYMAQLKCYY |
| | | AAGGACATCCCCTCCTTCGAGCGCTACATGGCCCAGCTCAAATGCTA | | FDYKEQLPESAYMHQLLGLNLLFL |
| | | CTACTTTGATTACAAGGAGCAGCTCCCCGAGTCAGCCTATATGCACC | | LSQNRVAEFHTELERLPAKDIQTN |
| | | AGCTCTTGGGCCTCAACCTCCTCTTCCTGCTGTCCCAGAACCGGGTG | | VYIKHPVSLEQYLMEGSYNKVFLA |
| | | GCTGAGTTCCACACGGAGTTGGAGCGGCTGCCTGCCAAGGACATACA | | KGNIPAESYTFFIDILLDTIRDEI |
| | | GACCAATGTCTACATCAAGCACCCAGTGTCCCTGGAGCAATACCTGA | | AGCIEKAYEKILFTEATRILFFNT |
| | | TGGAGGGCAGCTACAACAAAGTGTTCCTGGCCAAGGGTAACATCCCC | | PKIQITDYAKKRGWVLGPNNYYSF |
| | | GCCGAGAGCTACACCTTCTTCATTGACATCCTGCTCGACACTATCAG | | ASQQQKPEDTTIPSTELAKQVIEY |
| | | GGATGAGATCGCTGGGTGCATCGAGAAGGCCTACGAGAAAATCCTTT | | ARQLEMIV* |
| | | TCACTGAGGCCACCCGGATCCTCTTCTTCAACACACCCAAAAAGATG | | |
| | | ACAGACTACGCCAAGAAGCGAGGGTGGGTCCTGGGCCCCAACAACTA | | |
| | | CTACAGTTTTGCCAGCCAGCAGCAGAAGCCGGAAGACACCACCATTC | | |
| | | CCTCCACAGAACTGGCCAAACAGGTCATCGAGTATGCCCGGCAGCTG | | |
| | | GAGATGATCGTCTGA | | |
| prey56346 | 674 | GCCACCAACCCAGAAGCCCAAGAAGATTGTAAATGCCAAGAAAGATG | 675 | PPTQKPKKIVNAKKDVVNTKMFEE |
| | | TTGTGAACACAAAGATGTTTGAGGAGCTCAAGAGCCGTCTGGACACC | | LKSRLDTLAQEVALLKEQQALQTV |
| | | CTGGCCCAGGAGGTGGCCCTGCTGAAGGAGCAGCAGGCCCTGCAGAC | | CLKGTKVHMKCFLAFTQTKTFHEA |
| | | GGTCTGCCTGAAGGGGACCAAGGTGCACATGAAATGCTTTCTGGCCT | | SEDCISRGGTLSTPQTGSENDALY |
| | | TCACCCAGACGAAGACCTTCCACGAGGCCAGCGAGGACTGCATCTCG | | EYLRQSVGNEAEIWLGLNDMAAEG |
| | | CGCGGGGGCACCCTGAGCACCCCTCAGACTGGCTCGGAGAACGACGC | | TWVDMTGARIAYKNWETEITAQPD |
| | | CCTGTATGAGTACCTGCGCCAGAGCGTGGGCAACGAGGCCGAGATCT | | GGKTENCAVLSGAANGKWFDKRCR |
| | | GGCTGGGCCTCAACGACATGGCGGCCGAGGGCACCTGGGTGGACATG | | DQLPYICQFGIV* |
| | | ACCGGCGCCCGCATCGCCTACAAGAACTGGGAGACTGACATCACCGC | | |
| | | GCAACCCGATGGCGGCAAGACCGAGAACTGCGCGGTCCTGTCAGGCG | | |
| | | CGCCCAACGGCAAGTGGTTCGACAACCGCTGCCGCGATCAGCTGCCC | | |
| | | TACATCTGCCAGTTCGGGATCGTGTAG | | |
| prey82378 | 676 | GATATTGTATTTAATTATGTATATGCAATATTATAATATTATATATA | 677 | DIVFNYVYAIL*YYI*YQWYCIES |
| | | ATATCAATGGTATTGCATTGAGAGTTTACAAACAAGCCCTCACATTT | | LQTSPHISRQLIFNKGVKIIIXGK |
| | | CCAGACAACTTATTTTCAACAAAGGTGTCAAAATAATCATTGNGGGG | | ASSFQQMVVGNWVFRERGGNKNTP |
| | | AAATAATCATCTTTTCAACAAATGGTGGTGGGAAACTGGGTTTTCAG | | KKKCLEVLS*SIKQWXAT*GYLHA |
| | | AGAAAGAGGAGGGAACAAAAATACACCGAAAAAAAAATGTTTGGAAG | | TLQVLGLLPGP*XRAHSYXQXFAA |
| | | TNTTGTCCTAGAGCATCAAACAGTGGNGAGCCACATGAGGCTACCTT | | NPFXPKXFYGXAIXTLGX |
| | | CACGCCACACTCCAAGTTCTGGGGCTACTCCCAGGTCCCTGANATAG | | |
| | | AGCTCATTCCTATCNACAANCCTTTGCGGCGAACCCTTTCATNCCCA | | |
| | | AANGTTTTTATGGNTGNGCCATTGNTACTCTGGGGNNCTN | | |
| prey82384 | 678 | CNNCTTTTGNNGTATANAGGTGNANGNTAATTTGGGGGGCNATGAGA | 679 | XLLXYXGXX*FGGX*DCPRMDVXG |
| | | TTGCCCAAGAATGGATGTTCNTGGNNGANAACACTGNANNCTNTACA | | XXHXXLYTXXAXLXLXSYXXXAXX |
| | | CTCNGNATGCAGNGCTCNCACTGNGCAGCTACNACTNCNTGGCTGNG | | HXVGNDNLXT*FTXEEFA*ASXXI |
| | | TIAICCATGNTGTNGGGAATGACAATTTGCNCACATAATTTACTNAA | | ILX*PRQGI*TFXXTNGXXYEWRS |
| | | GAAGAGTTTGCNTGAGCGTCNNNTANTATTATTCTGTNCTGACCAAG | | XRXLTXVMVIXXTKLXXAXRXXQE |
| | | ACAAGGGATTTGAACNTTTNNCANCACAAATGGATANCANTACGAGT | | HLYYKDXNSPGLISL |
| | | GGAGGTCTNTACGCNNACTAACNANGGTAATGGTGATCTANNNAACA | | |
| | | AAATTGGNTTNTGCAGNNAGAAGNGANCAGGAGCACTTGTATTATAA | | |
| | | GGATTNAAATAGTCCTGGGTTAATCTCTTTATA | | |
| prey82387 | 680 | AAAAACTTTACTTTCCTTGGTTTTTTATAAGTNTAGCGGNCTCCAAC | 681 | KNFTFLGFL*V*RXPTHNVLGNNX |
| | | CCACAATGTCTTAGGAAATAATTNAACAATAGGAAAACCCTTAAAAC | | TIGKPLKPSISMQHTNIYM*VGIG |
| | | CATCTATCTCTATCCAACACACAAACATATACATGTGAGTGGGGATT | | R*IDRNCFPRRXXFXALXN*LXFX |
| | | GGTAGATAAATAGATAGAAATTGCTTTCCCAGGAGGANANCATTTNT | | IXXEG*XSXK*IPLXIHCXAXPNT |
| | | GGCTTTANTAAATTGATTANCTTTTTANATCAGNNAAGAAGGTTAAG | | IVXXAXIGKXWXXSXXPLSXXGPX |
| | | NATCTTTNAAATAAATCCCCTTATTNATTCATTGTNCTGCAANTCCT | | PXHQLXKMQDXSSAARAXXYXXKX |
| | | AACACAATAGTANTGGNAGCNNCNATTGGGAAAGNGTGGGANCNATC | | |
| | | CTANCNACCTTTGTCTNNNNNGGGGCCNNANCCTCNACACCAACTTT | | |
| | | TNAAAATGCAGGATGANAGCTCGGCGGCTCGNGCCNTNTTNTATNCC | | |
| | | NNAAAGNAN | | |
| prey82395 | 682 | TTTCTTCTTTCTTCTCCCAGATTTGCCTCTCCCCATTTCCATATCCA | 683 | FLLSSPRFASPIAIFHIQDYGVF* |
| | | AGATTACGGGGTATTTTAAAGCAACTCTCTTCTTATTCCTTTATTTT | | SNSLLIPLFFFYRGXIYIT*N*SF |
| | | TTTTTTATCGGGGTANAATATACATAACATAAAACTGATCATTTTAA | | *PFLXAPFXGIKXIHTIVQPLLLT |
| | | CCATTTTTAAGNGCACCATTCTGNGGTATTAAGTNCATTCACACTAT | | IXXTVXIFPN*NXVPIKQ*LSIXF |
| | | TGTNCAACCATTACTACTAACCATTTNTANAACTGTTTNTATCTTCC | | SFLSPF*FYFYFN*SYIXAKIIF* |
| | | CAAACTGAAACTNTGTACCCATTAAACAGTAACTCTCCATTTNTTTT | | KQXLIAFPYSNIKX |
| | | TCCTTTTTAAGCCCCTTCTAGTTCTATTTCTATTTTAATTAATCCTA | | |
| | | CATATTNGCTAAAATAATCTTCTGAAAACAANATTTAATTGCCTTTC | | |
| | | CCTATTCAAACATTAAATAN | | |
| prey45854 | 684 | CGAAGGTGAGGAGCCTGAGAACTTCTTCTGGGTGGGCATTGGGGCAC | 685 | EGEEPENFFWVGIGAQKPYDDDAE |
| | | AGAAGCCCTATGATGACGATGCCGAGTACATGAAACACACACGTCTC | | YMKHTRLFRCSNEKGYFAVTEKCS |
| | | TTCCGGTGCTCCAACGAGAAGGGCTACTTTGCAGTGACTGAGAAATG | | DFCQDDLADDDIMLLDNGQEVYMW |
| | | CTCCGACTTTTGCCAAGATGACCTGGCAGATGATGACATCATGTTGC | | VGTQTSQVEIKLSLKACQVYIQHM |
| | | TAGACAATGGCCAAGAGGTCTACATGTGGGTGGGGACCCAGACTAGC | | RSKEHERPRRLRLVRKGNEQHAFT |
| | | CAGGTGGAGATCAAGCTGAGCCTGAAGGCCTGCCAGGTATATATCCA | | RCFHAWSAFCKALA* |
| | | GCACATGCGGTCCAAGGAACATGAGCGGCCGCGCCGGCTGCGCCTGG | | |
| | | TCCGCAAGGGCAATGAGCAGCACGCCTTTACCCGCTGCTTCCACGCC | | |
| | | TGGAGCGCCTTCTGCAAGGCCCTGGCCTAA | | |
| prey82400 | 686 | AATGTGAAGATCCCGGCGGGCGTCGGCAATGGTCAACGCATCCGTCT | 687 | NVKIPAGVGNGQRIRLKCQCTPGE |
| | | GAAAGGCCAGGGGACGCCGGGCGAAAACGGCGGTCCAAATGGCGATT | | NGGPNGDLWLVIHIAPHPLFDIVG |
| | | TGTGGCTGGTGATTCATATTGCGCCACATCCGCTGTTTGATATTGTC | | QDLEIVVPVSPWEAALGAKVTVPT |
| | | GGCCAGGATCTGGAAATTCTGGTGCCGGTTAGCCCGTGGGAAGCGGC | | LKESILLTIPPGSQAGQRLRVKGK |
| | | GCTGGGTGCTAAAGTCACCGTTCCAACACTGAAAGAAAGCATTTTGC | | GLVSKKQTGDLYAVLKTVMPPKPD |
| | | TGACTATCCCGCCAGGCAGCCAGGCCGGGCAACCATTGCGCGTTAAA | | ENTAAL |
| | | GGCAAAGGTCTGGTGAGCAAAAAACACACCGCCGATCTCTATGCGGT | | |
| | | ACTGAAAATCGTGATGCCGCCGAAACCGGATGAAAACACTCCCGCGC | | |
| | | TG | | |
| prey29055 | 688 | CGGCAGGAGGTGGTCCAGGCCACGCTGGTGGTCCTGGCCTTGGCCAT | 689 | RQEVVQATLVVLALAMVPILLLGT |
| | | GGTGCCCATCCTGCTGCTTGCCACACCCCTGCACCTGCTGCACCGCC | | PLHLLHRHRRRLRRRPADRQEENK |
| | | ACCGCCGCCGCCTGCGGACGAGGCCCGCTGACCGACAGGAGGAAAAC | | AGLLDLPDASVNGWSSDEEKACGL |
| | | AAGGCCGGGTTGCTGGACCTGCCTGACGCATCTGTGAATGGCTGGAG | | IDDEEEAELVPSEVLMHQAIHTIE |
| | | CTCCGATGAGGAAAAGGCAGGGGGCCTGGATGATGAAGAGGAGGCCG | | FCLGCVSNTASYLRLWALSLAHAQ |
| | | AGCTCGTCCCCTCCGAGGTGCTCATGCACCAGGCCATCCACACCATC | | LSEVLWAMVMRIGLGLGREVGVAA |
| | | GAGTTCTGCCTGGGCTGCGTCTCCAACACCGCCTCCTACCTGCGCCT | | VVLVPIFAAFAVMTVAILLVMEGL |
| | | GTGGGCCCTGAGCCTGGCCCACGCCCAGCTGTCCGAGGTTCTGTGGG | | SAFLHALRLHWVEFQNKFYSGTGY |
| | | CCATGGTGATGCGCATAGGCCTGGGCCTGGGCCGGGAGGTGGGCGTG | | KLSPFTFAATDD* |
| | | GCGGCTGTGGTGCTGGTCCCCATCTTTGCCGCCTTTGCCGTGATGAC | | |
| | | CGTGGCTATCCTGCTGGTGATGGAGGGACTCTCAGCCTTCCTGCACG | | |
| | | CCCTGCGGCTGCACTGGGTGGAATTCCAGAACAAGTTCTACTCAGGC | | |
| | | ACGGGCTACAAGCTGAGTCCCTTCACCTTCGCTGCCACAGATGACTA | | |
| | | G | | |
| prey82412 | 690 | TTGNNNCAAGNCACCNGNANCCCNCNGACCNAAAAGCCCATNATNAT | 691 | LXQXTXXPXTXKPXXXVXDNNDDG |
| | | NGTCNGTGACAATAATGATGATGGGAGCCCAATGANGNNTGNTGNGC | | SPMXXXXLXSPLXTVXP*GGSVXX |
| | | TNNGAAGTCCCCTGNTNACAGTGNTACCATGAGGCGGCTCTGTTNTA | | L*XPGDGLPEXXICXTLLXLWLSX |
| | | NTACTTTGATNACCTGGAGACGGTCTGCCTGAANGGNANATATGCAN | | XXXX*PXX*X*LXIXRGLPCXXXQ |
| | | CACATTATTGGNTCTCTGGCTCTCTNTNCNTNCTANCNCNTAACCNG | | MXXXXXHXXTXXXXXXAXRXXSVL |
| | | ANGNCTGAGANTGACTNCNTATTNGGCGNGGGCTGCCCTGCNCANCC | | AXHXLHARGXAXXFXI |
| | | NTACAAATGNATGNGNANANNANGCACTNNNTGACNNNNTNNCNCNA | | |
| | | ACNGANGGCAGNCAGGTNCANATCTGTGTTGGCTNTCCACGNNTTGC | | |
| | | ATGCCCGAGGGNANGCTNCNNGGTTCNTGATCG | | |
| prey82423 | 692 | TTTTTTAGATACNNNNNNNNNNNNNNNNNNNNNNNNNNNNNNNNNNN | 693 | FFRYXXXXXXXXXXXXXXXXXXXX |
| | | NNNNNNNNNNNNNNNNNNNNNNNNNNNNNNNNNNNNNNNNNNNNNNN | | XXXXXXXXXXXXXTPGQFTV*MKL |
| | | NNNNNNNNNNNNNNNNNACACCTGGCCAGTTCACAGTTTAAATGAAG | | FPTLELLLNAVASNWHVLCTIRYS |
| | | CTCTTTCCTACTTTAGAACTTCTGCTAAATGCTGTAGCTTCCAACTG | | RYFRSRSIHLGTL*ELNVLKNQK* |
| | | GCATGTTCTTTGTACAATTAGGTATTCCCGATACTTCAGATCTAGAA | | CQPYRKCSIGXXXXXXXXXXXXXX |
| | | GTATACATTTAGGAACTTTATGAGAATTAAATGTACTGAAGAACCAA | | XXXXXXXX |
| | | AAATAATGTCAACCATATAGAAAGTGTTCAATAGGTNNNNNNNNNNN | | |
| | | NNNNNNNNNNNNNNNNNNNNNNNNNNNNNSINNNNNNNNNNNNNNNN | | |
| | | NNNNNNNNNNN | | |
| prey82425 | 694 | CCCAGCATAGGGATTAATGCAGGAACCATGGAGTTTGACTGATTTAG | 695 | PSIGINAGTMEFD*FSWTPGCLLV |
| | | TTGGACTCCAGGCTGCTTACTGGTTATATTCTACTTAACCTTTATTT | | IFYLTFICLSETIC*IGIKAHLPY |
| | | GCCTCAGTTTCTTCATCTGTTAAATAGGGATAAAATGACACCTACCT | | WVIEWIIGVNI*KYLRSNAWLLRW |
| | | TACTGGGTTATTGAGTGGATTATAGGTGTTAATATATGAAAATATCT | | LLLLLSKEKET*FSLD*PRSPCTT |
| | | TAGAAGCAATGCCTGGCTTCTAAGGTGGCTGTTATTGTTGTTATCCA | | ESVRSPDFIPSYYTRYAELQPSVA |
| | | AAGAGAAGGAAACATAATTTTCCTTAGACTAGCCTAGAAGTCCCTGT | | QCVPYGCSE |
| | | ACAACTGAGTCAGTCAGATCTCCAGACTTTATTCCTAGCTACTATAC | | |
| | | CAGGTATGCTGAGCTCCAGCCCAGTGTTGCTCAGTGTGTTCCATATG | | |
| | | GATGTTCTGAGAN | | |
| prey92611 | 696 | ACAGGCATGAGCCCCACCGTGCCCGCTGTGCCATTTCTTTTCTTTTT | 697 | TGMSPTVPAVPFLFFFFFXXGFXF |
| | | TTTTTTTNAAAANGGNTTTTNNTTTNNANCCCANGGNGGNNGGNANG | | XXXGGXXGXXXG*X*PXXPKXKXX |
| | | GGNCCNAANNNGGTTAANTTTAACCNTNGNCNCCCAAANTAAAGNNA | | LXXXXXXGXXXXGXPPXXXXFXXF |
| | | NTTTTANCNNANNACCNTTTNNGGGGGNGGGANTANNGCGGNNTNCC | | XXKXXFSXXXXXX*XXXPXXPXXA |
| | | CCCNNCCCNGGNTNATTTTTTNNNTTTTNAANAAAAANGGGANTTTT | | XQRXGXXXXXPPPXPKXXPFFXKX |
| | | CCNTNGNGGNCNGGNTNANTTAAAANNNCNGACCNNANGNNCCCNCC | | GX |
| | | NTGGCCNNCCAAAGGGNNGGGANTANNAANNNNANCCCCCCCCCCCN | | |
| | | NCCNAAANNAAANCCNTTTTTTNGAAAANAGGGTTNNTN | | |
| prey92612 | 698 | CCAGATTCTNATGATTTTAAGAATGTTTNGGCTATTCACATTTATTC | 699 | ADSXDFKNVXAIHIYSYIF*NDLI |
| | | ATATATATTTTGAAATGATTTAATCAAGGTCTGGGAACAAAAAGGTC | | KVWEQKGPVVIILNL*IK*REDNH |
| | | CAGTTGTGATTATATTGAATTTATAGATTAAATAAAGCGAGGATAAC | | LHNFESSYPRKRXXCYSEXLFLKR |
| | | CATCTTCACAATTTTGAATCTTCCTATCCAAGAAAAAGAAGNGNGTG | | FYFLLFIYIYFYFCFNXLSDYCXS |
| | | TTATTCTGAANCCCTCTTCTTAAAAAGGTTCTATTTCTTGCTATTTA | | LXPXXXXXXXXITXXRXIPFPLSX |
| | | TTTACATATACTTTTATTTCTGTTTTAACTTNCTAAGTGATTACTGT | | XRXXEPRPXLAXTXHXXSXPXXNN |
| | | TANTCACTTNTCCCTNTCGNGGNNTNGNCTNTACNCCANATAACCCN | | |
| | | CNATCGGNGNATTCCCTTTCCCCTATCAATNTTNAGGGNTTNGGAGC | | |
| | | CTCGCCCNNNCCTGCCNNTNTAAACCNNGCACAANNNGTCNGANCCT | | |
| | | NCCATNAATAACCT | | |
| prey92615 | 700 | GGGGGGGGNGNAAAANCNAATNTGCANATAATATATCCTCATGCACA | 701 | GGGXKXNXXIIYPHAHPXEKCITQ |
| | | TCCTNCTGAAAAATGTATCACACAAAAACANGCATTGTTGCCTTGCT | | KXALLPCCQHW*DSLTWIGYHSDS |
| | | GTCAACACTGGTAGGACTCATTAACCTGGATTGGGTACCACAGTGAC | | LILTTVPHILY*FI*LWS*PXEAH |
| | | TCATTAATTCTAACAACTGTGCCTCATATCCTCTATTAATTCATCTA | | VPPFELXALLXVHNRCPXQQAGST |
| | | GCTATGGAGCTAGCCANAAGAGGAACATGTGCCACCTTTTGAACTGC | | LHQ**RPS*LXQCDPLRXMLSLQT |
| | | ANGCTCTCCTAANAGTTCACAACAGATGTCCTTANCAGCAGGCAGGA | | HLMKWWP |
| | | TCAACTCTTCACCAGTAGTAAAGGCCTTCTTAGCTTTANCAATGTGA | | |
| | | TTAGCCACTAAGANTGATGCTCTCACTGCAGACACATTTGATGAAGT | | |
| | | GGTGGCCT | | |
| prey3341 | 702 | GTCCGTCGTGTCCTTTGATAAGGTCAAGGAGCCTCGGAAGTCAAGAG | 703 | SVVSFDKVKEPRKSRDSESHSRVR |
| | | ACTCAGAGTCCCATAGCAGGGTGCGTGAACGCAGTGAACGCGAACAA | | ERSEREQRMQAQWEREERERLBIA |
| | | CGCATGCAGGCGCAGTGGGAGCGCGAGGAGCGTGAGCGGCTGGAGAT | | RERLAFQRQRLERERMERERLERE |
| | | TGCCCGAGAGAGGCTGGCCTTCCAGCGCCAGCGGCTGGAGCGGGAGC | | RMHVEHERRREQERIHRBREELRR |
| | | GCATGGAGCGGGAACGGCTGGAGCGCGAACGCATGCACGTGGAGCAC | | QQELRYEQERRPAVRRPYDLDRRD |
| | | GAGCGCAGGCGCGAGCAGGAGCGCATCCACCGTGAGCGCGAGGAGCT | | DAYWPEAKRAGLDERYHSDFNRQE |
| | | GAGGCGCCAGCAGGAACTGCGCTATGAGCAGGAGCGGCGCCCCGCGG | | RFHDFDHRDRGRYPDHSVDRREGS |
| | | TGCGGCGGCCCTACGACCTGGACCGGCGAGATGATGCCTATTGGCCG | | RSMMGEREGQHYPERHGGPERHGR |
| | | GAAGCCAAGCGGGCCGGCCTGGATGAGCGCTACCATTCTGACTTTAA | | DSRDGWGGYGSDKRMSEGRGLPPP |
| | | CCGCCAGGAGCGCTTCCACGACTTTGACCACAGGGACCGCGGCCGCT | | PRRDWGDHGRREDDRSWQGTADGG |
| | | ACCCCGACCACTCGGTGGACAGGAGAGAAGGTTCAAGGTCAATGATG | | MMDRDHKRWQGGERSMSGHSGPGH |
| | | GGAGAACGAGAAGGACAGCATTACCCAGAACGCCATGGAGGACCAGA | | MMNRGGMSGRGSFAPGGASRGHPI |
| | | GCGCCACGGCCGGGACTCCCGCGATGGCTGGGGGGGCTATGGCTCTG | | PHGGMQGGFGGQSRGSRPSDARFT |
| | | ACAAGAGGATGAGCGAGGGCCGGGGGCTGCCTCCTCCCCCCAGACGT | | RRY* |
| | | GACTGGGGGGACCATGGCCGAAGAGAGGATGACCGGTCATGGCAGGG | | |
| | | CACGGCCGACGGGGGCATGATGGACAGGGATCACAAGAGGTGGCAAG | | |
| | | GTGGCGAGAGAAGCATGTCCGGTCACTCCGGGCCTGGCCACATGATG | | |
| | | AACCGAGGAGGAATGTCAGGGCGCGGCAGCTTTGCCCCAGGCGGGGC | | |
| | | CTCCCGGGGCCACCCCATCCCACACGGTGGCATGCAGGGCGGGTTTG | | |
| | | GAGGCCAGAGCCGGGGGAGCAGGCCCAGCGATGCCCGCTTCACTCGC | | |
| | | CGCTACTGA | | |
| prey92631 | 704 | TACAGGCATGAGCCACCACCGTGCCCGCTGTGCCATTTCTTTTCTTT | 705 | YRHEPPPCPLCHFFSFFFXKXXFX |
| | | TTTTTTTTTNAAAANGGNTTTTNNTTTNNANCCCAGGGNGGNNGGNA | | XXPRXXGXGPNXVXFNXXXPX*XX |
| | | NGGGNCCAAATNNGGTTAANTTTAACCNTNGCCNCCCAAANTAAACN | | FXXXXXXGGGXXGXXPXXXXIFXX |
| | | NANTTTTANCNAANNNCCNTTNNNGGGGGNGGGANTANNGGGGNNNN | | XXKXGXFPXXPGXFKXXXXXXPPX |
| | | CCCCCNNNCCNGGNTAATTTTTTNNNTTTTNAANAAAAANGGGANTT | | PPKGXXXXXXXPPPPXXXXXFXXK |
| | | TTCCNNNNNGGCCNGGNTNATTTAAAANNNCNGNCCNTANNNNCCCC | | RXXK |
| | | CCNNGGCCNCCCAAAGGGGNGGNANTANNAAANNNANCCCCCCCCCC | | |
| | | CCCCCNAAANNAAANCCNTTTTTTNNAAAAAAGGNNNNNNAAGAN | | |
| prey92641 | 706 | TGTTANNTNANNNATCATCNTGGGAGCTTCTCGANTGGGGGATGACG | 707 | CXXXXHXGSFSXGG*RXXXESWDT |
| | | GNCCNGANGNGAGTCTTGGGACACANGCCTTCATCTGACTCTACANG | | XLHLTLXADRAXASGVAXTLXAXS |
| | | CGGATCGGGCCNGGGCCTCNGGAGTATGANGCACTCTGGNAGCCNAG | | FQLXXXKXXXPLXLCLSXPXXXIG |
| | | AGCTTCCAACTCANNNCACANAAGATNATNCNNCCTCTTNTTCTTTG | | YEXXXXARNIPLXXMXFKXPXXXL |
| | | CCTCTCTTNNCCAANGTNATNTATAGGNTATGAGNGNNAACNNNNTG | | DLXXPXTXRXIXRXLKKXAKLEIF |
| | | CCAGAAACATACCTCTGANTNNNATGTGNTTTAAANTCCCNANTGNG | | XQMPHLXXCXCP |
| | | ANGCTNGATTTGNNNCNGCCNCNAACNNNCCGTTNNATTTNGAGANN | | |
| | | CCTTAAGAAGNTTGCCAAGCTNGAGATTTTTNAACAAATGCCTCATT | | |
| | | TANNGAGNTGTCNNTGTCCTT | | |
| prey92646 | 708 | CTTCCCGATGGATATAAGGTGCTGATGTNNTTGTGAGTNTAAGGTNA | 709 | LPDG*RC*CXCEXKVXXVXX*INX |
| | | TNTTNGTNTNCNATTGAATAAATGNTAAGTGTGCNTGGTTTTNANTC | | KCAWFXXMHXKPXLSXTXRXGXAW |
| | | ATGCACNNNAAACCCNGTCTNAGTNCCACGGANAGAANGGGCGANGC | | XXGXXXRSN |
| | | ATGGNTTNCTGGNTNGCNNTNCCGTTCTAAT | | |
| prey93191 | 710 | TTTAATGCTTCCAGATTTTCCATCACAGTTTATAATTTTTGTATTTA | 711 | FNASRFSITVYNFCI*VFWNLNWY |
| | | AGTCTTTTGGAATTTAAACTGGTATGATGTTAGACAATACAATATAT | | DVRQYNILITKCDFSL*FSFLCAG |
| | | TGATAACCAAATGTGATTTCAGTCTGTAATTTTCTTTTTTGTGTGCA | | FARFFFEHYVYFQERILKVFE*CP |
| | | GCGTTTGCCAGGTTTTTCTTTGAGCATTATGTGTATTTCCAAGAAAG | | GIFFIFKFYLFI*RQGLALLPRLK |
| | | AATTTTGAAAGTTTTTTTTTAATGCCCTGGAATATTTTTTATATTTA | | YTRQFCRLSLSXKTTSICHHTWLI |
| | | AATTTTATTTATTTATTTAGAGACAAGGTCTCGCTCTATTGCCCAGG | | XXXXXXXXXXXXXXX |
| | | CTTAAGTACACCAGGCAATTCTGCCGCCTTAGCCTCAGCNTGAAGAC | | |
| | | TACAAGCATATGCCACCATACCTGGCTAATTNNNNNNNNNNNNNNNN | | |
| | | NNNNNNNNNNNNNNNNNNNNNNNNNNNNNAC | | |
| prey92821 | 712 | TTCACGATGTGGTTTTTTTAGGCACTGCGTTAGAAGCGAAGAATAAT | 713 | FATMWFF*ALR*KRRIILIYLG*R |
| | | ATTAATTTATTTGGGTTAGAGAGTAGTGGTGAACCTGACTTGTAACA | | VVVNLTCNILFKRV*CSSL*LQLE |
| | | TTCTGTTTAAGAGAGTCTGATGTTCCAGTCTGTAACTGCAATTGGAA | | ELWKNDLWTHKILFKNIIFSASLK |
| | | GAGCTTTGGAAAAATGATTTGTGGACACATAAAATACTTTTTAAAAA | | KFVFLK*PALLLFFFFFXEXPXPR |
| | | CATAATTTTCTCTGCCTCCTTAAAGAAGTTTGTGTTTCTTAAATAAC | | XXGQGXXLT*LIAIPFPPGXRXXS |
| | | CAGCATTATTACTATTCTTTTTTTTTTTTTTNGAANCCCCTTNCCCC | | PPXPXXXWNXK |
| | | CGGNCAANNGGGCAGGGGNGNNACCTNACNTAATTGCACCCTTTCCC | | |
| | | NCCGGGGTTNAGGNATTTNTCCCCCCCCNCCCCCCNANNNANNTGGA | | |
| | | ATANCAAAAT | | |
| prey93166 | 714 | AACCAAAATGTCTCCAAATCCTGCCAAATGTTCCTGGAAGACAAAAT | 715 | NQNVSKSCQMFLEDKIYPLLQCIE |
| | | ATACCCTCTACTCCAGTGTATAGAATTACTGTACTAGAATATCATCT | | LLY*NIIFLKLIKWLYFLFPTADL |
| | | TTTTGAAATTGATTAAATGGTTATACTTTCTCTTCCCAACTGCTGAT | | *LEF*KCGL*LTW*VVAS*NCRFL |
| | | TTGTAGCTGGAATTTTAAAAGTGTCGTCTTTGACTTACTTGGTAGGT | | SLS*TY*IRNAEGRTQRSDFYKTL |
| | | GGTAGCTTCTTAAAATTCCAGGTTCCTGAGCTTATCCTAGACCTACT | | PSHPQPGNSALGLTLLWHNCCIIY |
| | | GAATCAGAAACGCTGAAGGTAGAACCCAGAGATCTGATTTTTACAAG | | IFFSCKSSW |
| | | ACACTACCTTCCCACCCCCAACCAGGTAATTCTGCCCTAGGCTTGAC | | |
| | | CCTCCTGTGGCATAACTGCTGCATTATCTACATTTTTTTCTCCTGCA | | |
| | | AGTCTTCCTGGN | | |
| prey48610 | 716 | ACCTGCGATTCACCAACATTGGTCCAGACACCATGCGTGTCACCTGG | 717 | LRFTNIGPDTMRVTWAPPPSIDLT |
| | | GCTCCACCCCCATCCATTGATTTAACCAACTTCCTGGTGCGTTACTC | | NFLVRYSPVKNEEDVAELSISPSD |
| | | ACCTGTGAAAAATGAGGAAGATGTTGCAGAGTTGTCAATTTCTCCTT | | NAVVLTNLLPGTEYVVSVSSVYEQ |
| | | CAGACAATGCAGTGGTCTTAACAAATCTCCTGCCTGGTACAGAATAT | | HESTPLRGRQKTGLDSPTGIDFSD |
| | | GTAGTGAGTGTCTCCAGTGTCTACGAACAACATGAGAGCACACCTCT | | ITANSFTVHWIAPRATITGYRIRH |
| | | TAGAGGAAGACAGAAAACAGGTCTTGATTCCCCAACTGGCATTGACT | | HPEHFSGRPREDRVPHSRNSITLT |
| | | TTTCTGATATTACTGCCAACTCTTTTACTGTGCACTGGATTGCTCCT | | NLTPGTEYVVSIVALNGREESPLL |
| | | CGAGCCACCATCACTGGCTACAGGATCCGCCATCATCCCGAGCACTT | | IGQQSTVSDVPRDLEVVAATP |
| | | CAGTGGGAGACCTCGAGAAGATCGGGTGCCCCACTCTCGGAATTCCA | | |
| | | TCACCCTCACCAACCTCACTCCAGGCACAGAGTATGTCGTCAGCATC | | |
| | | GTTGCTCTTAATGGCAGAGAGGAAAGTCCCTTATTGATTGGCCAACA | | |
| | | ATCAACAGTTTCTGATGTTCCGAGGGACCTGGAAGTTGTTGCTGCGA | | |
| | | CCCCCAC | | |
| prey78599 | 718 | CTGCGAGCTGAGCGAGCAGCGCGGGAGCTTCTCGGATGGGGGAGACG | 719 | CELSEQRGSFSIDGGDGPTGSPGD |
| | | GGCCCACGGGGAGTCCAGGGGACACAGGCATCCCTCTGACCTACAGG | | TGIPLTYRRSGPVPPEDDASGSES |
| | | CGGAGCGGGCCGGTACCTCCCGAGGATGATGCCTCTGGCAGCGAGAG | | FQSNAQKIIPPLFSYRLAQQQLKE |
| | | CTTCCAGTCCAACGCCCAGAAGATAATCCCACCTCTGTTCAGCTACC | | MKKKGLTETTKVYHVSQSPLTDTA |
| | | GCCTTGCCCAGCAGCAGTTAAAGGAGATGAAAAAGAAAGGTCTGACG | | IDAAPSAPLDLESPEEAAANKFRI |
| | | GAAACTACCAAAGTGTATCACGTGTCTCAGAGTCCCCTGACAGACAC | | KSPFPEQPAVSAGERPPSRLDLNV |
| | | TGCCATTGATGCGGCCCCCAGCGCTCCCTTAGATTTGGAAAGCCCGG | | TQASCAISPSQTLIRKSQARHVGS |
| | | AAGAAGCTGCAGCAAACAAGTTCCGGATCAAATCCCCATTTCCGGAG | | RGGPSERSHARNAHFRRTASFHEA |
| | | CAGCCCGCGGTCAGTGCCGGGGAAAGGCCTCCCTCCAGGCTGGATCT | | RQARPFRERSMSTLTPRQAPAYSS |
| | | AAATGTGACTCAGGCCAGTTGTGCCATAAGCCCCAGCCAGACTCTGA | | RTRTCEQAEDRFRPQSRGAHLFPE |
| | | TCCGCAAGTCACAGGCAAGGCACGTGGGCAGCAGAGGGGGCCCGTCC | | KLEHFQEA |
| | | GAAAGGAGCCATGCCAGGAACGCCCATTTCAGGAGGACAGCGAGTTT | | |
| | | CCATGAAGCCAGGCAGCCCCGGCCGTTCCGAGAGAGGAGCATGTCCA | | |
| | | CTCTGACTCCACGGCAGGCCCCTGCCTACAGCTCTAGGACGCGGACC | | |
| | | TGCGAGCAGGCAGAGGACAGATTTAGGCCTCAGAGTCGAGGTGCCCA | | |
| | | CCTGTTTCCTGAAAAACTGGAGCATTTCCAAGAGGCA | | |
| prey92693 | 720 | CACAACCGAGTGTTGAGTAAAAGAAATCAGATGCAAAAGTACATCCT | 721 | HNRVLSKRNQMQKYILYGSI**HS |
| | | ATATGGCTCCATTTAATGACATTCAAGAACAAGTCAAACTAAGCAGT | | RTSETKQWCLRVVST**NFLKKKK |
| | | GGTGTTTAAGGGTGGTGTCTACTTAGTAAAACTTCTTAAAAAAAAAA | | RQXGXP*GIXXXXXXRGVXXXLXG |
| | | AAAAGGCAAANGGGGTNNCCNTAAGGGATCANTTNNTNTTTNNTTTT | | XFRXPXGXPXXXFXXGVXXXFPXX |
| | | NAGGGGNGTGGNGGNGNTTTTANTGGGNNGNTTTAGGNNCCCCCNGG | | NLVXXXLFSLCXLLIXWYYKTAPX |
| | | GCTNNCCTNTTNTTTNTTTTTNAANNGGGGTNNNANAANNTTTTCCN | | XFLFSPXXDVLATPXXGXKLXXXX |
| | | TTNNAAAATTTGGTTANCCNNCNACTTTTTTCTCTNTGTNNGCTCCT | | GXXV |
| | | NATTTNGTGGTATTATCGTACCGCCCCCNNANNNTTTTTATTTTCCC | | |
| | | CTTNAANAGATGTTTTAGCNACCCCCTNNGNCGGTTTNAAGCTCCNT | | |
| | | NGNCNCANNGGNNTTNTTGTNTG | | |
| prey92698 | 722 | NNNNNNNNNNNNNNNNNNNNNNNNNNNNNNNNNNNNNNNNNNNNNNN | 723 | XXXXXXXXXXXXXXXXXQIFFKCE |
| | | NNNNCAGATTTTTTTTAAATGTGAATTTCTAGCATATTTATAAAAAT | | FLAYL*KYNYFYKEWDNIFSACYP |
| | | ATAACTACTTTTATAAGGAATGGGATACATCTTTTCTGCATGTTATC | | TFIXKNTF*TR*LLVHFIYWVSVI |
| | | CAACTTTCATTTANAAAAATACTTTCTAAACAAGGTAGTTGTTAGTA | | MFYTLK**KLLYINLERXSG*XL* |
| | | CATTTTATTTACTGGGTAAGTGTCATAATGTTTTACACATTAAAATG | | NAXFSLTDFSXL*NVILNFTREPN |
| | | ATAAAAATTATTGTATATTAATCTTGAAAGGNATTCTGGTTAGNGCC | | LLXGKFW* |
| | | TTTAAAATGCTNTCTTTTCACTAACAGATTTTAGTNATCTGTGAAAT | | |
| | | GTTATATTGAATTTTACAAGAGAACCNAATTTACTTTNAGGTAAATT | | |
| | | TTGGTGAN | | |
| prey92714 | 724 | TATATTATATAGTATAAGGGAATAATATGGTACTTCTCACCTTACTG | 725 | YII*YKGIIWYFSPYWAN*IPVNN |
| | | GGCAAATTAAATACCTGTCAATAATTTAAGGATTTCCTTGTAATACA | | LRISL*YK*SFFLKYRSSELDLFS |
| | | AATAATCTTTTTTTTTAAAATATAGAAGTTCTGAGTTAGACCTGTTT | | SE*WAKLP*NSLYILNGNGSKTPE |
| | | AGCTCAGAATAGTGGGCTAAACTACCATAAAATTCTCTGTATATCTT | | NHQLITHLQISAWVGGDSQVPMII |
| | | AAATGGTAATGGGTCAAAAACTCCAGAAAATCATCAGTTGATAACAC | | *PQ*IKLGNTHICYSDTALDL*NS |
| | | ACCTACAGATAAGTGCATGGGTAGGAGGGGATAGCCAAGTGCCCATG | | IY |
| | | ATAATTTGACCTCAGTAAATTAAACTGGGCAATACACATATTTGCTA | | |
| | | TTCTGATACTGCATTAGACTTATAAAATTCCATCTAC | | |
| prey92741 | 726 | NNNNNNNNNNNNNNNNNNNNNNNNNNNNNNNNNNNNNNNNNNNNNNN | 727 | XXXXXXXXXXXXXXXXXXXXXXXX |
| | | NNNNNNNNNNNNNNNNNNNNNNNNNACCTATCTAGGAAAATTTTAAA | | TYLGKF*IAYVVHILFLLDSTGLE |
| | | TTGCATATGTAGTTCACATTCTATTTCTATTGGACAGCACTGGTCTA | | V*VGHGRSLPPRLPQTFIQVIVIP |
| | | GAAGTGTAGGTTGGCCATGGGCGGAGCCTACCTCCCAGGCTTCCTCA | | IPSVFQSFFLYSSQISEW*SVGHL |
| | | GACCTTCATACAAGTGATTGTCATTCCTATCCCCTCAGTTTTTCAAT | | LSWVYHLGKPV*NTGTGEPSP*SL |
| | | CATTCTTCCTTTATTCCTCCCAAATCTCAGAGTGGTAGAGTGTAGGC | | I*KDWTG |
| | | CACTTGCTGAGCTGGGTGTATCACCTGGGGAAGCCTGTTTAAAATAC | | |
| | | AGGCACTGGAGAACCCTCTCCATAAAGTCTGATTTAGAAACACTGGA | | |
| | | CTGGA | | |
| prey92744 | 728 | TGCCCAACTGGCCATCGCTNATGCCGCCTCTTCCTCTGGACCCACCC | 729 | CPTGHRXCRLFLWTHPLLLSCPIK |
| | | NCTGCTACTGAGCTGCCCAATCAAAGCCNAGTCTGCTATCCNGGTNT | | AXSAIXVSWPEGGMYXGSCDGPPF |
| | | CTTGGCCAGAGGGGGGCATGTATATNGGCTCCTGTGATGGGCCCCCT | | ASLSCLHQGAKKNTVLERLVLSKQ |
| | | TTTGCCTCCCTCTCTTGCTTACATCAGGGGGCCAAGAAAAACACTGT | | KTFGKCWTCIISLLGTPQEELVPV |
| | | TCTGGAGAGACTTGTATTGAGCAAGCAAAAAACATTTGGAAAGGGGT | | LGRLPTCPCPFEDCCLPCLLQTVG |
| | | GGACTTGCATCATCTCCCTACTTGGGACACCCCAAGAAGAGCTTGTG | | IPIXG |
| | | CCAGTGCTGGGGCGCCTGCCCACATGCCCTTGCCCTTTTGAGGACTG | | |
| | | TGGGTTGCCTTGTTTGCTACAGACTGTTGGCATCCCCATCTNGGGC | | |
| prey92745 | 730 | TGCTGCANAAATNNTGTGTGGTCTGTTCCCACTGCCATTAACCCAAC | 731 | CCXNXVWSVPTAINPTLTFSACPG |
| | | CTTGACTTTCTCTGCCTGTCCTGGTATAGACATTTAGTTCTTGTTAA | | IDI*FLLSTVFIIFNLYQPKTTKE |
| | | GCACAGTTTTCATTATTTTCAACCTTTACCAACCAAAAACTACTAAA | | KVNILRSTVKRYPNILTESXXYKG |
| | | GAAAAAGTTAATATTCTGAGGAGTACAGTTAAAAGATATCCTAACAT | | *HVKQARAVFFKLKQ*GPGVNKTT |
| | | TCTCACTGAATCANCCNAATATAAAGCCTAACATGTTAAACAGGCTA | | FQEVRIV*GESG*RWSASTVLPEL |
| | | GAGCAGTTTTCTTCAAACTAAAGCAGTAGGGCCCTGGAGTTAACAAG | | DLSXNSCGLL |
| | | ACAACTTTCCAGGAGGTTAGGATAGTTTAAGGAGAATCCGGTTAAAG | | |
| | | GTGGTCTGCTTCCACCGTACTCCCAGAGCTTGATCTGTCTNAGAATT | | |
| | | CTTGTGGTTTGCTA | | |
| prey92747 | 732 | TCTAGCTTGTGATGTGGAGTTTTTTGGTTTTTATAAANATGATACTC | 733 | SSL*CGVFWFL*X*YSPAIIF*IF |
| | | TCCAGCCATTATTTTTTAAATATTTCTTCCTTCATGTTGTTCTTCTG | | LPSCCSSGTPLHAF*TI*LFSIAT |
| | | GAACTCCNTTGCATGCCTTTTAAACTATTTGATTATTTTCAATAGCT | | ACFVLFLKKNF*LG*FYXPI*KFS |
| | | ATTGCATGCTTTGTTCTTTTTTTAAAAAAAAATTTTTAGTTGGGATA | | DSFFSXVQXCK*ACRRNSLI*CLL |
| | | ATTCTATNGACCAATCTAAAAATTCAGTGATTCCTTTTTCAGTTCNC | | FLPXPLNSLVNXSFLXGXCPLFPF |
| | | TTCAGNCCTGCAAATGAGCTTGCCGAACAAATTCTTTGATATAATCC | | XSXXKXSGNRLC*RXAXXXXXQXX |
| | | CTTTTATTTCTACCATNTCCCTTCAACTCTCTNGTGAATTNCTCATT | | GXQYP |
| | | TCTTCANGGANGTTGTCCACTTTTTCCATTTAGNTCCCNNCNAAAAN | | |
| | | CATCAGGNAACAGGTTATGTTAAAGGNCGCCTTTNCNNCANANAGNC | | |
| | | CAANCNNCGCGGGNTCAATATCCN | | |
| prey92938 | 734 | GGGAAATGGAAATATACTGTTCTTACATTATACGTGAAATGGTATGC | 735 | GKWKYTVLTLYVKWYGMI*RQIVI |
| | | TATGATTTCAAGGCAGATTGTGATTAATTTAAACCTGCAAACCACAG | | NLNLQTTE*PLKSNQ*SIAK*AKS |
| | | AGTGACCACTAAAATCAAACCAATCAAGTATACCTAAATAAGCAAAG | | GNKTEP*KILNESKTRQXXXXXXX |
| | | AGTGGAAATAAAACAGAACCCTAAAAAATACTCAATGAATCTAAAAC | | XXXXXXXXXXXXXXXXXXXXXXXX |
| | | AAGGCAAGANNNNNNNNNNNNNNNNNNNNNNNNNNNNNNNNNNNNNN | | XXXXXXXXXXXXXXXXXXXXXXXX |
| | | NNNNNNNNNNNNNNNNNNNNNNNNNNNNNNNNNNNNNNNNNNNNNNN | | XXXXXXXXXXXXXXXX |
| | | NNNNNNNNNNNNNNNNNNNNNNNNNNNNNNNNNNNNNNNNNNNNNNN | | |
| | | NNNNNNNNNNNNNNNNNNNNNNNNNNNNNNNNNNNNNNNNNNNNNNN | | |
| | | NNNNNNNNNNNNNNNNNNNNNNNNNNNNNNNNN | | |
| prey92796 | 736 | NTGCCGCTGGAACCCTGTGTGGTTTCTCTAAATCCAGGAAACATGTC | 737 | XPLEPCVVSLNPGNMSMQVVARIL |
| | | TATGCAGGTTGTGGCACGTATACTACCTAAATTCACTGAATAAATAG | | PKFTE*IVKVSG*DYPSTTFKKIC |
| | | TAAAGGTTAGTGGGTGAGACTATCCCTCCACGACATTTAAAAAAATT | | YDFLRQGLTVLLKLVSNS*AQDTL |
| | | TGTTATGATTTTTTAAGACAGGGTCTCACTGTGTTACTCAAGCTGGT | | LPLPPKMLGLRT*ATVPNPWXYFK |
| | | CTCGAACTCCTAGGCTCAAGACACCCTCCTGCCTTTGCCTCCGAAAA | | PTFYSSXKXSSSVVAXFIKCSIFX |
| | | TGTTGGGATTACGGACATGAGCCACTGTGCCCAACCCTTGGNGGTAT | | LAYISEIXRQXLGYHPPXSPLXLV |
| | | TTTAAGCCCACTTTTTACACTAGTGNAAAANACAGTAGTTCTGTTGT | | DAXXGS |
| | | GGCTGANTTCATAAAATGCAGCATTTTCNCATTGGCCTACATTTCAG | | |
| | | AAATTANTAGGCAAGNATTAGGGTACCATCCCCCACNCTCCCCTTTG | | |
| | | GNGCTNGTTGATGCCTTNAGNGGGTCAAG | | |
| prey92805 | 738 | TTTTATCCCAGGGAAGTTATAGAAGTAAAAACTCCATTCGAACCCAT | 739 | FYPREVIEVKTPFEPMEQKTTDIY |
| | | GGAGCAGAAAACCACCGACATCTACTCTATGAGTGCTGGGCCTCCTG | | SMSAGPPGACGINTNLWIL*GEFL |
| | | GGGCGTGTGGTATAAATACCAACCTTTGGATCTTGTAAGGTGAGTTT | | ISCKYKACTE*LGQTVSWDKLPMK |
| | | TTAATCAGCTGCAAATATAAAGCTTGCACAGAATGATTAGGGCAGAC | | QKEPEVYSSHSYLASSQGETHLRT |
| | | TGTCAGCTGGGACAAGCTCCCAATGAAGCAAAAAGAGCCTGAAGTGT | | SAVLGKPTIALMRMEFPPKRVLER |
| | | ACAGCTCTCACTCATATCTGGCTTCTTCACAAGGGGAAACTCATCTG | | |
| | | AGGACATCAGCTGTTCTTGGTAAACCTACCATCGCTCTGATGAGGAT | | |
| | | GGAATTTCCCCCAAAAAGAGTCTTAGAGAGG | | |
| prey92806 | 740 | AGTGCTGGCTNTTTATTTAATAGGTGGAAATTATATTCATACATTTA | 741 | SAGXLFNRWKLYSYI*SNQSQRFS |
| | | AAGCAATCAATCTCAGCGTTTTTCTGTTCTGTAAACTTTAATCCCAT | | VL*TLIPCQRQATKEKKGLKTYFY |
| | | GCCAGAGACAAGCTACTAAGGAGAAGAAAGGTCTTAAGACTTACTTC | | LKEKHHIRASNRKWKHYRKL*LLL |
| | | TACCTGAAAGAAAAGCATCATATAAGAGCAAGTAACAGAAAATGGAA | | LIPSFIIFLSF*K*F*ISTFXYYL |
| | | ACACTACCGTAAACTCTGACTTCTTTTACTCATTCCTTCTTTTATTA | | CE*XXWQXKQXYXXLLXVXYXAXX |
| | | TTTTCCTGTCTTTTTAAAAGTAGTTTTAAATATCTACTTTTTNATAC | | XX |
| | | TACTTATGTGAGTAAANAAANTGGCAACNGAAGCAAANATACNGGAN | | |
| | | NTTGTTGCNGGTNNTTTATNNCGCNNANTANNTNTNAT | | |
| prey92808 | 742 | ACCACCACCACCACCACATGGCGGCCGATTTTTGNNNNNNNNNNNNN | 743 | TTTTTTWRPIFXXXXXXXXXXXXX |
| | | NNNNNNNNNNNNNNNNNNNNNNNNNNNNNNNNNNNNNNNNNNNNNNN | | XXXXXXXXXXXXXXXXXXXXXXXX |
| | | NNNNNNNNNNNNNNNNNNNNNNNNNNNNNNNNNNNNNNNNNNNNNNN | | XXXSC*XXXSLAHTNILFXXAVLV |
| | | NNNNNNNNNNNNTCTGGCTAANGANTATNTTCCTTAGCACATACNAA | | KTIWXWXIVXXLXXGMXARRXXXL |
| | | TTTATTTNAATNAGCTGTGCTGGTNAAAACAATATGGANGTGGNNGA | | XXXXLXXXXXX*FSRXXGLSLRXV |
| | | TTGTATNNNNTTTGNCTNNAGGNATGNACGCACGCAGAANCNCNTNC | | N |
| | | CTATNGCNNGNGGNGCTNNATANANTGGNCNTTANTTAATTTTCTCG | | |
| | | AANANNNGGNCTNTCCCTNCGTNGNGTAAATGG | | |
| prey92816 | 744 | GGACGNNGGGCAGAAGGAAGGAAGGAGAAGGCAGGCAGGAAATCAAT | 745 | DXGQKEGRRRQAGNQYDSIAWELR |
| | | ATGACAGCATAGCATGGGAGCTAAGAACACACCTTCTAGAAGCAGAC | | THLLEADSMNSNPGSTIYKLYFPL |
| | | TCTATGAATTCAAATCCTGGGTCTACCATTTATAAGCTGTACTTTCC | | ANMNLQYLLLTDCYKDEMC*YR*S |
| | | TCTGTAAAATATGAATCTACAGTACCTACTTCTCACAGATTGTTATA | | KHSVNMRH*PEPNKVPSSNIIIEC |
| | | AAGATGAGATGTGTTAATATAGATAAAGTAAGCACTCAGTGAATATG | | ITFFSLQSKFTYSISFGS |
| | | AGGCATTAACCAGAACCCAACAAGGTGCCATCATCAAATATAATAAT | | |
| | | TGAGTGCATAACTTTTTTCAGTTTACAAAGCAAATTTACATATAGCA | | |
| | | TTTCTTTTGGATCCC | | |
| prey92825 | 746 | TAAAATACACCAAAACACTCCACTTCACTGGGCAGTTGCAGCAGGAA | 747 | KIHQNTPLHWAVAAGNVNAVDKLL |
| | | ATGTTAATGCAGTTGATAAGCTTTTGGAAGCTGGTTCTAGCCTGGAT | | EAGSSLDIQNVKGETPLDMALQNK |
| | | ATCCAGAATGTTAAGGGAGAAACACCTCTTGATATGGCTCTACAAAA | | NQLIIHMLKTEAKMRANQKFRLWR |
| | | CAAAAATCAGCTCATTATTCATATGCTAAAAACAGAAGCCAAAATGA | | WLQKCELFLLLMLSVITMWAIGYI |
| | | GAGCCAACCAAAAGTTCAGACTTTGGAGGTGGCTGCAGAAATGCGAG | | LDFNSDSWLLKGCLLVTLFFLTSL |
| | | CTCTTCCTGCTGCTGATGCTTTCTGTGATTACCATGTGGGCTATTGG | | FPRFLVGYKNLVYLPTAFLLSSVF |
| | | ATACATATTGGACTTCAATTCAGATTCTTGGCTTTTAAAAGGATGTC | | WIFMTWFILFFPDLAGAPFYFSFI |
| | | TTCTAGTAACACTGTTTTTTCTGACATCTTTGTTTCCAAGGTTCTTG | | FSIVAFLYFFYKTWATDPGFTKAS |
| | | GTTGGGTATAAGAACCTTGTATACTTACCAACAGCCTTTCTGCTAAG | | EEEKKVNIITL |
| | | TTCTGTTTTTTGGATATTTATGACTTCGTTCATCTTATTTTTTCCTG | | |
| | | ATTTAGCAGGAGCCCCTTTCTATTTCAGTTTCATTTTCAGCATAGTA | | |
| | | GCCTTTCTATACTTTTTCTATAAGACTTGGGCAACTGATCCAGGCTT | | |
| | | CACTAAGGCTTCTGAAGAAGAAAAGAAAGTGAATATCATCACCCTTG | | |
| prey92845 | 748 | GAGCTGCAACGGTCTAGGATCCTTTCTGGGGCGCCCTTCAACACCAA | 749 | ELQRSRILSGAPFNTNRCWGSDVL |
| | | CAGGTGTTGGGGCTCCGATGTGTTGGGGTTCTTATGAATAGACAACT | | GFL*IDNCEGLLRHKHWMVGCVAV |
| | | GTGAAGGATTGTTGAGACATAAACATTGGATGGTTGGATGTGTGGCA | | HGGKQSFTYYLVFAIKDPRSLLSE |
| | | GTCCATGGTGGAAAACAAAGCTTCACTTACTATTTAGTATTTGCTAT | | L*SILHL*HTLNATEKKKKXPL*F |
| | | AAAAGACCCAAGATCCCTTCTTTCTGAACTCTGAAGTATCTTACACC | | XXNXXXFVXXKKKXXNFQXXGLGX |
| | | TGTAACATACTTTGAATGCTACAGAAAAAAAAAAGNNACCCCTNTAA | | XXXTLTSXPXPXSPX |
| | | TTTTTNCNNAATTNCCNANCTTTTGTTNANNTTAAAAAAAAACANNC | | |
| | | AAATTTTCAAAANANAGGGTTGGGGNAANATTTNTNTACACTTACNT | | |
| | | CCGNCCCCNTCCCAAANTCCCCANACC | | |
| prey92860 | 750 | TCCTTGCAANCGATGNGNACCATGCGCANCNNATGNANCNGAGTGGT | 751 | SLQXMXTMGXXXXXVVXWXYPXGX |
| | | NNNATGGNCTTATCCNANTGGAAANNTCACCGGNTNNCTGAGANAGA | | XTGXLRXXXFVXHXRPHXPXGWSX |
| | | NGGANTTTGTGGNCCACNATCGACCTCACGNTCCCTGNGGATGGAGC | | XGGX*PXXXSXXLXDADXRLXLWX |
| | | GNNNCTGGNGGATNCTGACCATNNTNAANATCGTNNAGNTTATGNGA | | XXPXXPXPVLSXGPVCVXXXVCSX |
| | | CGCTGACCNGCGCCTTNCNCTNTGGNCGGNAGNACCTGNATNTCCTG | | SXPLGXHAXXXECXXXXXKAGXAX |
| | | NCCCTGTCCTTTCGCANGGACCNGTTTGTGTCCNGNCCANAGTATGC | | |
| | | TCCCANTCGNCTCCCCTAGGACNACATGCCNCCTTNTNNGAGTGCTN | | |
| | | AANGNCTCNTCNAAAAGCTGGNNNAGCGCNCT | | |
| prey92861 | 752 | CTNAGGCACCTCGGTTGGTTTTGAGCACATCTTGCCAGTTCTGTGTC | 753 | LRHLGWF*AHLASSVSIWGSFK*N |
| | | TATTTGGGGATCTTTTAAATAAAATTCTGATTCATACTCAATATAAT | | SDSYSI*LGSAAPGFMPANGKIHS |
| | | TAGGATCTGCAGCCCCAGGCTTCATGCCTGCAAATGGAAAAATCCAT | | SSWNEMIRKGKENEWNEIKIL*SH |
| | | TCATCTAGTTGGAATGAAATGATCAGAAAGGGGAAGGAGAATGAGTG | | SLDATRECIILESLIS*RAKDSYR |
| | | GAATGAAATTAAAATATTATAATCTCACTCCCTGGATGCCACAAGGC | | ETQI*MSVTPDSNISM*ISPILPN |
| | | AGTGCATCATTCTGGAAAGCCTAATTTCCTAGCGAGCTAAGGATTCA | | KEWWDLRRTGECLSSLKEFG |
| | | TACCGACAAACACAAATTTAAATGTCTGTAACCCCTGACAGTAACAT | | |
| | | AAGCATGTGAATCAGCCCAATACTGCCCAACAAGGAGTGGTGGGACC | | |
| | | TGCGAAGGACTGGAGAGTGCCTGTCTAGCCTAAAGGAATTCGGAT | | |
| prey92878 | 754 | TTTGTGCCATTAGAATGGGTGGTTTTTAGGGTATATGGAATTATTTA | 755 | FVPLEWVVFRVYGII*TTSLKFSK |
| | | AACAACATCCCTAAAATTTTCAAAAAGATTTTTCGTCACAAAATATT | | RFFVTKYCWKMQMDXVDKIRTIRI |
| | | GTTGGAAAATGCAAATGGATAANGTAGATAAAATTAGGACAATAAGA | | FF*CYIKKNL*DPPL |
| | | ATTTTTTTTTAATGCTACATAAAAAAAAACCTGTAGGACCCCCCCCT | | |
| | | GG | | |
| prey92886 | 756 | GGGGGGGCTACGTTACATTATGGCAGAAAAAAAGTGCAACTGACATA | 757 | GGATLHYGRKKVQLTYYKEIF*V* |
| | | CTACAAAGAGATTTTTTAAGTTTAAAAAAAGTTTGGATCTTTTGGAT | | KKFGSFCFLFFFLXFXXXKXPXXX |
| | | TTCTTTTTTTTTTTTTGGNNTTTNNGGNCNTAAAAANCCCNNAATTN | | LXPXXRXXXFFFXGPPQXXXXXXF |
| | | NAATTANCCCCCCNAANAAGGGNNGNTTTNTTTTTTTTTTTNGGNCC | | SXXXXXXFXXXXFPKXXPXXXPXX |
| | | CCCCCAAAANANCCNNNNANGGGNNTTTTCCNTNCNCCNCCNTNCCN | | XPLXKPXXKXXXARGPXGXXXXXX |
| | | NGTTTNGNNNNNNCCNNTTCCCAAAGGNGCNCCCCNTTNTTNNCCCC | | XXXXXPP |
| | | CNANGGGNNCCCTTGNNGAAACCNNCNANNAAAANCNGGCNTGCAAG | | |
| | | GGGGCCCNGGGGAANCCNNNCCCNTTTNNNTTANNAAAANCNGGNCC | | |
| | | CCCCCC | | |
| prey92905 | 758 | CATGCCCGGTCACTCCGGAAATCCTCTGTGTCTGGTTCATCGAGGTC | 759 | HARSVRKSSVSGSSRSSRKLYSGS |
| | | TTCCAGGAAATTATATTCTGGATCATCATCATCATCTGCTTCATCTA | | SSSSASSRKKKCFRCSLTLFSNF* |
| | | GGAAAAAAAAGTGTTTCAGATGCTCATTAACTCTGTTCTCAAACTTC | | ESVPIKHXXXXXXXXXXXXXXXXX |
| | | TAAGAATCTGTTCCAATAAAGCACATNNNAAANNNNNNNNNNNNNNN | | XXXXXXXXXXXXXXXXXXXXXXXX |
| | | NNNNNNNNNNNNNNNNNNNNNNNNNNNNNNNNNNNNNNNNNNNNNNN | | XXXXXXXXXXXXXXXXXXVYSMKI |
| | | NNNNNNNNNNNNNNNNNNNNNNNNNNNNNNNNNNNNNNNNNNNNNNN | | ILSVKATCTVKLTVKKGXAX |
| | | NNNNNNNNNNNNNNNNNNNNNNNNNNNNNNNNNNNNNNNNNNNNNNN | | |
| | | NNNNNNNNIANNNNNAGTATATTCCATGAAAATAATACTTTCTGTAA | | |
| | | AAGCTACTTGTACAGTGAAGCTGACAGTGAAAAAAGGAGNGGCCCNT | | |
| prey92907 | 760 | GGGTGACTCAGGGAAAGGGATTTTATATTTCACCATATACCCTCTTA | 761 | GDSGKGILYFTIYPLI*FISVVGK |
| | | TATAGTTTATCTCAGTGGTAGGAAAACATTTACTGTAAAGGGCCAGA | | HLL*RAR*IFYALWP*GLGHNSSV |
| | | TAGTAAATATTTTATGCTTTGTGGCCATAGGGTCTTGGTCACAACTC | | LLL*HESSHRHYVN**VWLCFQ*N |
| | | CTCAGTTCTGCTGTTATAGCATGAAAGCAGCCACAGACATTATGTAA | | FIYKQSGEPDLADGP*FAHP*FTF |
| | | ATTAATGAGTGTGGCTGTGTTTCCAATAAAACTTTATTTACAAACAG | | PLRTCIAFMIRISLIETS*LKIK* |
| | | AGTGGTGAGCCAGACTTGGCTGATGGGCCATGATTTGCCCACCCCTA | | |
| | | GTTTACATTCCCCCTCAGAACATGCATTGCTTTTATGATAAGAATTA | | |
| | | GTTTAATTGAAACTAGTTAATTAAAAATAAAATAAAG | | |
| prey92908 | 762 | ATGTCTGTGGCCGGTGGGGAGATTCGTGGGGACACGGGGGGAGAGGA | 763 | MSVAGGEIRGDTGGEDTAAPGRFS |
| | | CACTGCTGCTCCCGGCCGGTTCAGCTTCAGCCCGGAGCCCACGCTCG | | FSPEPTLEDIRRLHAEFAAERDWE |
| | | AGGACATCCGCCGCCTCCATGCTGAGTTTGCTGCGGAACGAGACTGG | | QFHQPRNLLLALVGEVGELAELFQ |
| | | GAACAGTTCCATCAGCCTCGGAATCTCCTCCTGGCCTTGGTTGGGGA | | WKTDGEPGPQGWSPRERAALQEEL |
| | | AGTGGGGGAGCTGGCAGAACTCTTTCAGTGGAAAACCGATGGGGAAC | | SDVLIYLVALAARCRVDLPLAVLS |
| | | CTGGCCCCCAAGGCTGGTCCCCCAGGGAACGGGCAGCCCTTCAAGAG | | KMDINRRRYPAHLARSSSRKYThL |
| | | GAGCTTAGTGACGTCCTCATCTACCTGGTGGCATTAGCAGCCCGCTG | | PHGAISEDQAVGPADIPCDSTGQT |
| | | CCGTGTGGATCTGCCGCTAGCAGTGCTCTCCAAAATGGACATCAACC | | ST* |
| | | GGCGACGCTACCCAGCCCATCTGGCCCGCAGCTCTTCCCGCAAGTAT | | |
| | | ACAGAATTGCCCCATGGGGCCATCTCTGAAGACCAGGCTGTGGGGCC | | |
| | | TGCGGACATTCCCTGTGACTCCACAGGCCAGACCTCAACCTAG | | |
| prey56687 | 764 | CGTCTCCTTGCTGGCAGCGTCCGAGGCTGAGGTGGAGCACCTCCTGT | 765 | VSLLAASEAEVEQLLSERAPLTGH |
| | | CCGAGAGAGCCCCGCTCACGGGGCACAGCTGCTACCCAGAGGCCCTG | | SCYPEALLHFRTHCFNWHSPTYEY |
| | | CTGCACTTCCGGACCCACTGCTTCAACTGGCACTCCCCCACGTACGA | | ALRHLYVLVNLCEKPYPLHRIKLS |
| | | GTATGCGTTGAGACATTTGTACGTGCTGGTCAACCTTTGTGAGAAGC | | MDHVCLGHY* |
| | | CGTATCCGCTTCACAGGATAAAATTGTCCATGGACCACGTGTGCCTT | | |
| | | GGTCACTACTGA | | |
| prey92929 | 766 | ATACATCTCCCAAAAACGGATTGTGTGTTCCTGGGTTTTCTTTAACT | 767 | IHLPKTDCVFLGFL*LHK*LNKNK |
| | | GCACAAGTAGCTGAATAAAAACAAGAGATTTTTACTCTTTTTTCTTT | | RFLLFFLLETEVSXXXXXXXXXXX |
| | | TAGAGACAGAAGTCTCGTNNNNNNNNNNNNNNNNNNNNNNNNNNNNN | | XXXXXXXXXXXXXXXXRGGITILT |
| | | NNNNNNNNNNNNNNNNNNNNNNNNNNNNNNNNNNNNNNNNNNNNNNN | | I*FLYLLPAESFLRSKILGRTQDM |
| | | NNNCCGAGGTGGAATCACCATCTTGACCATATGATTTTTATATCTTT | | KQIKWNYYD*RREGRVQEL*ILAF |
| | | TACCAGCAGAGTCTTTCTTACGAAGTAAAATTTTAGGTAGAACTCAA | | SFPF |
| | | GATATGAAACAGATAAAGTGGAACTACTATGACTGAAGGAGGGAGGG | | |
| | | AAGAGTCCAAGAACTGTAAATTTTAGCCTTCTCTTTCCCTTTCCC | | |
| prey92933 | 768 | AGGACCACTATTTTAGGAANCACATTCTTGGCATACTTTGAATGAAT | 769 | RTTILGXTFLAYFE*IFTSLELDC |
| | | TTTTACTTCTCTNGAGCTNGATTGCAAAGAANAGAAATNTCCCNCCN | | KEXKXPXXXSKAFWKNYXVNXLHN |
| | | GCNTTAGTAAAGCATTCTGGAAAAACTATNGGGTAAATCNNTTACAC | | CNCKNXYV*VYXFSYEGXXLCHXX |
| | | AATTGTAATTGCAAAAATGNGTATGTGTAAGTGTATATNTTTTCTTA | | *FKXWXGXGS*XLSXAXXLXDFXL |
| | | CGAAGGGNGANAATTGTGTCATTNTNCTTAATTCAAACNCTGGNNGG | | XXTXXXFTLXXLGRXXXS |
| | | GNNGGGGATCTTAANCTTTGTCNAGNGCANATNCNCTNGNAGACTTT | | |
| | | TNCTTGGNGTNGACCNTTNGCCANTTTACNCTGTNTNAACTTGGGCG | | |
| | | GCNGCNCNACTCC | | |
| prey92941 | 770 | ACAAAAGCCTATGGTTTCTTTTAAAAAGTTTCATCTTGAGCTAATGC | 771 | TKAYGFF*KVSS*ANATV*IKCMK |
| | | TACAGTTTAAATAAAATGTATCAAAGGTAGTTATCAAAAACAATTTG | | GSYQKQFVYLKFYADIPFLQRIIA |
| | | TATATTTAAAATTCTATTTTGACATCCCTTTTTTACAGCGAATTATA | | GYVKFKNKF*EIFLSCK*EL*IYL |
| | | GCAGGTTATGTGAAATTTAAAAATAAATTTTAGGAAATTTTTTTATC | | S*K*DLCFYSDILKV*KHFVCYFG |
| | | TTGTAAGTAAGAATTATAAATCTATCTCAGTTAAAAGTAGGATCTTT | | IS*V*SAGKGNLFYAKFF*FVKCF |
| | | GTTTTTATTCAGATATTTTGAAAGTTTAGAAGCATTTTGTATGCTAC | | *CVYFL |
| | | TTTGGTATTTCTTAAGTATAGTCAGCAGGAAAGGGTAACCTGTTTTA | | |
| | | TGCAAAATTTTTTTAATTTGTCAAATGTTTTTAGTGTGTCTACTTTC | | |
| | | TNA | | |
| prey92954 | 772 | NNNNNNNNNNNNNNNNNNNNNNNNNNNNNNNNNNNNNNNNNNNNNNN | 773 | XXXXXXXXXXXXXXXXXXXXXXXX |
| | | NNNNNNNNNNNNNNNNNNNNNNNNNNNNNNNNNNNNNNNNNNNNNNN | | XXXXXXXXXXXXXXXXXXXXXCIL |
| | | NNNNNNNNNNNNNNNNNNNNNNNNNNNNNNNNNNNNNNNNNTGTATT | | SLYVCICRCKLSNLKLQINYSVTY |
| | | CTCTCTTTATATGTGTGTATATGTAGATGCAAGCTTTCCAATTTAAA | | FELESQG*PCNSLIDPILINIKSI |
| | | ATTACAAATTAATTACTCAGTTACATATTTTGAATTGGAATCGCAAG | | IL*CRYFLIWNSITTCLILLIISP |
| | | GTTGACCCTGTAACTCTTTAATAGATCCAATTCTCATAAACATTAAA | | |
| | | AGTATAATTCTATAATGCAGATATTTCTTAATATGGAATTCTATTAC | | |
| | | TACTTGTCTGATTCTTTTGATTATTTCCCCA | | |
| prey92956 | 774 | CTTCCCTGTGCTCTCCTGCACTCCAAGGCTGGCAGCGGCCTTCCCAC | 775 | LPCALLHSKAGSGLPTVLHPISLP |
| | | TGTTCTGCACCCAATCTCTCTGCCCCTGCCTTCCACAGCCTGTGCTG | | LPSTACAAFLDSPTLSELPGPCPS |
| | | CCTTCCTGGACTCACCCACCCTCTCTGAGCTGCCAGGACCCTGCCCT | | ALSRFSSPTSFKPTKRCKNSTMNT |
| | | TCTGCCCTCTCCCGCTTCTCTTCTCCTACAAGCTTCAAGCCTACAAA | | LISFI*VHQLLTWHHFAAXXCHRL |
| | | AAGGTGCAAGAATAGTACAATGAATACCCTTATATCTTTCATCTAAG | | FXLXACXETGSHSVPQAETEVXTX |
| | | TTCATCAGTTGTTGACATGGCACCACTTTGCTGCANCNNNATGCCAT | | LGCTXNPRXX |
| | | AGGCTNTTTNNGCTANCCGCATGTTTNGAGACAGGGTCNCACTCTGT | | |
| | | NCCCCAGGCTGAGACTGAAGTGCNNNCACGNATCTNGGGTGCACTGN | | |
| | | CAATCCCCGNGNTNAG | | |
| prey92957 | 776 | AATACTCTCTTGGATTCAGGTATGCTTTTCTTTGGCTGGGTGTAGTT | 777 | NTLLDSGMLFFGWV*LLCL*CYSW |
| | | ACTGTGTTTGTGATGTTACTCTTGGACCTCAGATAAGCCTGGAAGGT | | TSDKPGRSPSFLLCRELMDKVEFL |
| | | CACCATCTTTCCTGTTGTGCAGAGAGCTGATGGACAAAGTTGAATTT | | AAAIYLMSTVPYLDYHSGPHLYGN |
| | | CTGGCTGCCGCCATATACCTGATGTCAACTGTACCATATCTAGATTA | | AFMGVHQSRTCFS*VPLLQ*GFSF |
| | | TCACTCAGGCCCACACCTTTATGGGAATGCCTTTATGGGAGTTCATC | | MALSS*NKEGKRRGFNYFVLFRET |
| | | AGAGCAGAACCTGCTTCTCTTAGGTTCCTCTTCTTCAGTGAGGCTTT | | WVIHQNISLGEVLSATEYNFLEMI |
| | | TCCTTCATGGCTTTGTCCTCCTGAAATAAAGAGGGTAAGAGAAGAGG | | HFEVTVCDL*TLAFDTI |
| | | TTTTAATTATTTTGTCTTATTTAGCGAGACATGGGTAATACATCAGA | | |
| | | ACATTTCTCTAGGTGAGGTTCTTTCAGCTACAGAATATAATTTTTTA | | |
| | | TTTATGATACATTTCGAAGTAACTGTATGCGACTTGTGAACACTGGC | | |
| | | CTTTGACACCATC | | |
| prey92959 | 778 | CAGTTGGTATCGTTAGGGAACTTTTAAGAGAAACATTTCGTTTATGC | 779 | QLVWLGNF*EKHFVYANNS*RMYL |
| | | AAACAACTCTTAAAGGATGTATCTTTCAAACATTTCTTTGGTGCTGG | | SNISLVLVRSLTTGLIGVVVSSLL |
| | | TCAGGTCACTTACTACTGGGCTAATAGGAGTGGTCCTTTCCTCCTTG | | KLSVT*EYLGLIEKLVVGVLFAEK |
| | | TTAAAGTTGTCTGTTACCTGACAGTATTTGGGACTGATAGAGAAGCT | | AAGTKFTG*LEMXXXXXXXXXXXX |
| | | GGTGGTGGGGGTGTTATTTGCAGAGAAAGCAGCTGGCACTAAGTTTA | | XXXXXXXXXXXXXXXXXXXXXXXX |
| | | CAGGCTAATTAGAAATGNNNNNNNNNNNNNNNNNNNNNNNNNNNNNN | | XXXXXXXXX |
| | | NNNNNNNNNNNNNNNNNNNNNNNNNNNNNNNNNNNNNNNNNNNNNNN | | |
| | | NNNNNNNNNNNNNNNNNNNNNNNNNNNNNNNNNNNNNNNNNNNNNNN | | |
| | | NNNNNNNNNNNNG | | |
| prey93006 | 780 | NGNAGGNNANACNNAAAACTNCCGGNCTNAATCTTNCGGTCTTANAC | 781 | XRXXXKLPXXIXRSXTSXHLXLXE |
| | | TTCTNANCATCTCNGATTANGCGAGTGGACAACNATGCCCNATATCG | | WTTMPXIDFGMLXSALPFXXXXXH |
| | | ATTTTGGAATGCTANATTCTGCCTTGCCTTTTTANANCNATNANCNT | | TFGXXKKLFFLXQGLPLSPKRGXQ |
| | | CACACTTTTGGATNCNTAAAAAAACTTTTTTTTTTGANACAGGGTCT | | XXXLAXXKXXXSXSXMXXATXPVX |
| | | CCCTCTCTCGCCCAAGCGGGGGNTGCAGTNCNNNGANCTCGCTGNGN | | LIXXPCHXLXYTXXFFXR*LLXGE |
| | | NAAAANNGTTNNCATCNNTNTCTTNCATGATNNGNGCCACGNCCCCN | | TLXL |
| | | GTGNTNCTTATCATNNTGCCTTGCCATTANCTCANGTATACTCNCNA | | |
| | | ATTCTTCAGNAGGTGACTCCTTTGNGGTGAAATCCTGNGGCTGT | | |
| prey93089 | 782 | TGGGGGACACAGATTATAGCAATTATTTAGATATCTTGATAATTTAT | 783 | WGTQIIATI*IS**FIYNHNHIGD |
| | | ATATAACCATAACCATATAGGTGATAGGAGATAGTAGAAATATATAG | | RR**KYIV*GLSYSAMHINTTHTH |
| | | TATAAGGTCTTTCATATTCAGCCATGCACATAAACACCACACACACA | | THTDTHVHIHVHKYLGIQILVCXL |
| | | CACACACACACAGACACACACGTCCACATACATGTTCATAAATATTT | | SCSHQPGSKMLSSISFMISAKSPM |
| | | AGGTATACAGATAGTTTGCATNCTTTCCTGCTCTCACCAGCCTGGGT | | FLIILPCH*LKYTPKFFSSDSFVX |
| | | CAAAAATGTTATCATCAATTTCTTTCATGATAAGCGCCAAGTCCCCA | | KSCGCIWXRL |
| | | ATGTTTCTGATCATTTTGCCTTGCCATTAGCTCAAGTATACTCCCAA | | |
| | | ATTCTTCAGCAGTGACTCCTTTGTGGNGAAATCCTGTGGCTGTATTT | | |
| | | GGNGCAGATTA | | |
| prey92962 | 784 | GGGAATCACTCTNTTGCCGNNNNNNNNNNNNNNNNNNNNNNNNNNNN | 785 | GNHSXAXXXXXXXXXXXXXXXXXX |
| | | NNNNNNNNNNNNNNNNNNNNNNNNNNNNNNNNNNNNNNNNNNNNNNN | | XXXXXXXXXLRSWDYGHVPLYPAX |
| | | NNNACCTNAGGAGCTGGGACTACGGGCATGTACCACTGTACCCTGCT | | LLDRVSLFCPNWSGTPXLM*SAGL |
| | | NATCTTTTAGATAGGGTTTCACTATTTTGCCCAAACTGGTCTGGAAC | | GFPXHWDYGSEPGRQXRXDF*FTM |
| | | TCCGGANCTCATGTAATCCGCCGGCCTCGGCTTCCCGNAGCACTGGG | | GFWGAEPXXXXASVLILPXXGXXX |
| | | ATTATGGGAGTGAGCCAGGCAGACAGNGNCGGATNGATTTTTGATTT | | XMFGDXGXSVCXXX |
| | | ACNATGGGTTTTTGGGGGGCGGAACCCTNCTNTCANNGAGCATCTGT | | |
| | | NCTCATATTGCCNTNAGNAGGGNGGANTNGGTGNATGTTCGGGGATN | | |
| | | CTGGNNGGAGTGTNTGCNTTNGCNNG | | |
| prey92971 | 786 | AGACTTACTCACTGTCACAAGAACAGCAGGGGAAAGACCTGCCCCCA | 787 | RLTHCHKNSRGKTCPHDSFTXLLG |
| | | TGATTCATTTACCTNCTTACTGGGTCCCTCCCATAANAGATGGGATT | | PSHXRWDFKMRFGWGHSPTIXXII |
| | | TCAAGATGAGATTTGGTTGGGGACACAGCCCAACCATATNANTCATA | | YPPL*KIFKYXLFIVYLEXIFVXG |
| | | ATATATCCCCCTCTTTAAAAAATATTTAAATATTTNCTTTTTATAGT | | MR*RSHFKFFPNSEPIVPVLFIEY |
| | | CTACTTAGAANTTATTTTTGTGNGGGGTATGAGATAGAGATCCCATT | | RPFPXDXXXVPLKHIRFQKXXGXX |
| | | TTAAATTTTTTCCAAATTCAGAACCNATTGTACCAGTATTGTTTATT | | SWXLXGPX**I* |
| | | GAATACCGTCCTTTCCCCNGTGATNTGCNGNTGGTACCTCTNAAACA | | |
| | | CATCAGATTTCAGAAAATNCNNGGATNANTTTCCTGGNTGCTTCANG | | |
| | | GTCCCTNATGATAAATTTAATN | | |
| prey22857 | 788 | TTAACACCTTCGTGCTGATGCAGCTCTTCAATGAAATCAACTCCCGA | 789 | NTFVLMQLFNEAINSRKTHGEKNV |
| | | AAGATCCATGGAGAGAAGAACGTCTTTTCAGGCATCTACCGCAACAT | | FSGIYRNIIFCSVVLGTFICQIFI |
| | | TATCTTCTGCTCTGTAGTCTTGGGCACATTCATCTGCCAGATTTTCA | | VEFGGKPFSCTSLSLSQWLWCLFI |
| | | TCGTGGAATTTGGGCGTAAACCCTTCAGTTGTACAAGCCTCAGCCTG | | GIGELLWGQFISAIPTRSLKFLKE |
| | | TCTCAGTGGCTGTGGTGTCTCTTCATTGGGATTGGAGAACTTCTGTG | | AGHGTTKEEITKDAEGLDEIDHAE |
| | | GGGCCAGTTCATCTCCGCAATACCTACCCGATCCCTGAAGTTCCTGA | | MELRRGQILWFRCLNRIQTQIKVV |
| | | AGGAGGCTGGGCATGGCACCACCAAAGAGGAGATCACCAAGGATGCC | | KAFHSSLHESIQKPYNQKSIHSFM |
| | | GAGGGACTGGATGAGATTGACCATGCTGACATGGAGCTGCGCCGAGG | | THPEFAIEEELPRTPLLDEEEEEN |
| | | CCAGATCCTCTGGTTCCGGGGCCTGAACCGTATCCAGACTCAGATCA | | PDKASKFGTRVLLLDGEVTPYANT |
| | | AAGTGGTCAAAGCGTTCCATAGTTCCCTCCACGAAAGCATTCAGAAA | | NNNAVDCNQVQLPQSDSSLQSLET |
| | | CCCTACAACCAAAAGTCCATCCACAGCTTCATGACCCACCCTGAATT | | SV* |
| | | CGCCATAGAGGAGGAGTTGCCACCAACACCACTCCTGGATGAGGAAG | | |
| | | AGGAGGAAAATCCTGACAAGGCTTCTAAGTTTGGGACTAGGGTGCTC | | |
| | | CTGTTGGATGGTGAGGTCACTCCATATGCCAATACAAACAACAATGC | | |
| | | GGTGGATTGCAACCAAGTGCAGCTCCCCCAGTCCCACAGCTCTCTAC | | |
| | | AGAGCCTAGAGACATCAGTTTGA | | |
| prey92975 | 790 | ACCGGGCAAGCTACGTTACATTATGGCAGAAAAAAAGTGCAACTGAC | 791 | TGQATLHYGRKKVQLTYYKEIF*V |
| | | ATACTACAAAGAGATTTTTTAAGTTTAAAAAAAGTTTGGATCTTTTG | | *KKFGSFGFLFFFXXFXXLKXXXX |
| | | GATTTCTTTTTTTTTTTTNGGNTTTTNNGNNNCTAAAAANNNGNNAA | | XFPXXXRXXXFFFLXXXQRGXEXX |
| | | TTNTNNTTCCCCNCCCNAGANAGGANANNNTTNTTTTTTTTTTTGGN | | FXXXPPXXXXXXVPXPQXSXXXXX |
| | | NNCCNNCCAAAGGGGNNNNGAANGGGNTTTTNNCCNTCNCCCCCCCN | | XXRXAXXXXPXXXXXXXRXXXRXX |
| | | NTNCCNNNNNTNGNCNNGTCCCNNCNCCCCAGGNATCNNNCCNTNCN | | XAXWFXXDXXXXXWXTFX |
| | | NTCNCCNTCNCGAGGNGNGCCNNNGNGNAAAIWCCCNNNNAAANNCN | | |
| | | GGNNNNNGNACGGNNCNCNNGGAGAANAANTGNCGCTTNTTGGTTTN | | |
| | | GGNNAGACNCCNNGNCGNCCNNCTGGNTNACCTTCNTT | | |
| prey92976 | 792 | GGGGGAAAGACATTAGTCTTATATTCCACATTTTAAAAATTCTTATT | 793 | GGKRLVLYSTF*KFLFSTACICIS |
| | | TTCAACGGCTTGTATCTGTATTTCTCCATATTTTGGAGTAATTATCT | | PYFGVIICMICVGAVAFYLRSRIS |
| | | GCATGATATGTGTAGGTGCTGTGGCTTTTTACCTCAGAAGCAGAATC | | FPVHLRVMMPNL*WSVSETQSLQC |
| | | TCATTCCCAGTACATTTGAGAGTCATGATGCCAAATCTTTGATGGTC | | VCSQWCGEYASSHLEKCLRKPGQC |
| | | TGTGTCAGAGACACAAAGTCTACAATGTGTGTGTTCACAGTGGTGTG | | PCVFTIIFLCIYGDDSVIFWDMDQ |
| | | GATTTTATGCCTCAAGTCACTTAGAAAAGTGTCTTAGAAAACCTGGA | | CVSLHTHIXHHLYVASQ |
| | | CAGTGCCCTTGTGTCTTCACAATAATTTTTTTGTGTATTTATCGAGA | | |
| | | TGATTCTGTAATCTTTTGGGACATGGACCAATGTGTATCTCTTCATA | | |
| | | CTCATATTNTGCACCATCTGTATGTAGCAAGTCAG | | |
| prey92977 | 794 | GGCGTGTGCAGAGAGGGGAGGCGGCCTGGTGGGAAAAGTCTANAACA | 795 | GVCREGRRPGGKSLXQREYDGPMH |
| | | GAGAGAATATGATGGCCCAATGCACCCCACAATGGCCACCAGGAAAC | | PTMATRKPRXAAGDLCTILVIFL* |
| | | CCAGGGNGGCTGCAGGAGATCTTTGTACTATTCTTGTCATTTTTCTG | | ISHRFK*FFNME*NVLKKAQ*GLL |
| | | TAGATCTCACATCGCTTCAAATAATTTTTTAATATGGAATAAAATGT | | CAAXRVVLRLKY**IQKVLFKLXX |
| | | GTTAAAAAAAGCACAATAAGGTCTGCTCTGCGCAGCANACAGGGTTG | | PIXGSGSLXXXKVXVXSLGRXEHX |
| | | TTCTGAGACTGAAATACTAATAGATACAAAAAGTACTTTTTAAACTA | | RERHH*HQI*IPG |
| | | TNAANACACANACGTGAAAGGCATCATTAGCACCAAATATGAATACC | | |
| | | NCCNATCAGNGGGTCTGGTTCCCTNANTANANAGAAAGTCNNGGTCN | | |
| | | TTTCCCTTGGGAGGNCNGAGGGG | | |
| prey92979 | 796 | ATCTTGGTCCCATTTATATAAAATGTCCAGGAAGTACAAATCTNTAG | 797 | ILVPFI*NVQEVQIXRDXMLLT*L |
| | | AGACNGCATGCTACTTACTTAATAATTACCTGGGGCTGAGGGTTGAG | | PGAEG*VCIDCIWL*RIFLGYKNV |
| | | TAGGGATTGATTGCATATGGCTATAAAGGATATTTTTGGGTTATAAA | | LKLVMVIIAQLXKFTKDIILIVYS |
| | | AATGTTCTAAAACTGGTTATGGTGATAATCGCACAACTCTNTAAATT | | *HSITQV*TV*VHLLVDFFQPTSI |
| | | TACTAAAGATCATTTAATTGTATACAGTTGACACTCGATAACACAGG | | ESTIFVGCETHVCGGPTFLMWEFH |
| | | TTTAAACTGTATGAGTCCACCTACTTGTGGATTTTTTTCAGCCAACT | | GAHEGT*VYSDFGIGRESLETIPQ |
| | | TCAATAGAAAGTACAATATTTGTGGGATGTGAAACCCATGTATGTGG | | IYHGMTILNTPIISSLSS*SDYIN |
| | | AGGGCCTACTTTTCTTATGTGGGAGTTCCATGGGGCTCACTTTGGGA | | P*TPFLGESGRS**VKVKNLGL*V |
| | | CTTGAGTATACTCTGATTTTGGTATAGGCAGAGAGTCCCTGGAAACA | | CGQ |
| | | ATCCCTCAGATATACCACGGGATGACTATACTTAATACCCCAATTAT | | |
| | | TTCCTCCCTTTCATCCTGATCTGACTACATAAATCCTTAAACCCCCT | | |
| | | TTTTGGGGGAGTCAGGGAGGAGTTGATAGGTAAAGGTTAAGAATTTG | | |
| | | GGTCTGTGAGTATGTGGGCAG | | |
| prey92981 | 798 | AGATAAGGACACTTAGCAAATGTAAAAATAAAATCTAACTCTCATTT | 799 | R*GHLANVKIKSNSHLTSRERKVR |
| | | GACAAGCAGAGAAAGAAAAGTTAGATTTTATTTTTACATTTGATCAG | | PYFYI*SVPIFLGVW*IFIYKCLF |
| | | TTCCTATATTTCTAGGAGTTTGGTGAATATTTATTTATAAATGCTTA | | FSKPS*NRALLEDFINEFCNALWS |
| | | TTTTTTTCCAAGCCAAGTTAGAATAGAGCACTTTTAGAGGATTTCAT | | *ENITYT*HTLIDTQTQIEIS*LS |
| | | AAATGAATTTTGCAATGCTCTCTGGAGTTAAGAAAATATCACATATA | | S*NFSHESGINILMVNFRHLLDRI |
| | | CCTAACATACATTAATAGATACACAAACACAAATAGAGATTTCATAG | | ERHT*LVKHDFSHESGINILMVNF |
| | | CTTTCATCCTGAAATTTCAGCCATGAATCAGGCATAAATATTCTGAT | | RHLLEWIERH |
| | | GGTTAATTTTACACATCTACTTGATCCGATTGAGAGACACACATAGC | | |
| | | TTGTCAAACACGATTTCAGCCATGAATCAGGCATAAATATTCTGATG | | |
| | | GTTAATTTTAGACATCTACTTGAGTGCATTGAGAGACACAG | | |
| prey92989 | 800 | CCTCTCATTATTATCTGTTATGGTGATCTTTGATATTACTATTATTA | 801 | PLIIICYGDL*YYYYYNP*TTLM* |
| | | CAATCCATGAACCACACTCATGTAAGAGTGAACTTCATTGATCAATT | | E*TSLINFVCVLTTPLTGCSPTFL |
| | | TTGTGTGTGTTCTAACTACTCCACTGACTGGCTGTTCCCCCACCTTT | | PSLSXLPIP*DTILKLGQLITLQW |
| | | CTCCCATCTCTCTCANAACTCCCTATTCCCTGAGATACAATATTAAA | | QWPLSVQVKGKVXSLSR*TKKLET |
| | | ATTAGGCCAATTAATCACCCTACAATGGCAATGGCCTCTAAGTGTNC | | IXLMRKACQKLR*VES*ASCAIQL |
| | | AAGTGAAAGGGAAAGTCNCATCTCTCTCACGTTAAACCAAAAAGCTA | | AKL |
| | | GAAACGATTAANCTTATGAGGAAGGCATGTCAAAAGCTGAGATAGGT | | |
| | | TGAAAGCTAGGCCTCTTGTGCCATACAGTTAGCCAAGTTGTN | | |
| prey92990 | 802 | TCTTCCACCAAGACAGAGACGTTAGCAACGCATGGCGGGTGGGGACC | 803 | SSTKTETLATHGGWGPGVLRRGYP |
| | | TGGGGTCCTCAGGAGGGGGTACCCGGGGCCCCGGCCAGAGATACATC | | GPRPEIHQLHPRGGTADGSQHQQD |
| | | AATTACACCCCCGTGGGGGGACAGCCGATGGGAGCCAGCACCAGCAG | | PRAPRTEVCPTHFLPTTCAPKSRA |
| | | GATCCGAGGGCGCCCCGGACAGAGGTCTGCCCCACCCACTTCCTCCC | | CPGRWRPGVECTCSHEVLGVF*ES |
| | | CACCACCTGTGCCCCAAAGAGCAGGGCCTGCCCGGGAAGGTGGCGTC | | LSDTTLV*PHRVVSNIHGSGYGMV |
| | | CTGGAGTCGAGTGTACCTGCAGCCATGAGGTTCTGGGTGTTTTTTGA | | YLCNL |
| | | GAGAGTCTGAGTGACACCACACTCGTGTGACCCCACAGGGTTGTGTC | | |
| | | CAACATACACGGAAGTGGCTATGGAATGGTGTATTTGTGCAACCTGG | | |
| | | N | | |
| prey93095 | 804 | CTTGTCTTATATGCCCCTTTGTAATTCCATTGCCTAGCACAGCAGAT | 805 | LVLYAPL*FHCLAQQMLSLIGLIL |
| | | GTTGTCACTAATACGTTTGATTCTGTGATTTGGATCCAATGTCATTG | | AFGSNVIVTGYFFPHNRLFLYDFM |
| | | TGACAGGATACTTTTTCCCCCATAATAGGTTATTTTTATATGACTTT | | PTKPKDKTH*IENNFLYYKVIWFL |
| | | ATGCCCACAAAACCCAAAGATAAAACACATTAAATTGAAAATAATTT | | FAVLSSFYKIGAVLPSFL*KTWFG |
| | | CTTGTATTATAAGGTGATTTGGTTCCTATTTGCAGTTCTTTCTTCTT | | PDILTETKLPAFFF*KLLLV*LNC |
| | | TTTATAAAATAGGTGCTGTTTTACCTAGTTTCTTATGAAAAACATCG | | TAME*AQINLLSTAWLCSQIVLKC |
| | | TTTGGGCCAGATATCTTGACAGAAACCAAATTACCAGCATTTTTCTT | | KP*SPQGR |
| | | TTAAAAATTATTGTTGGTTTAGCTGAATTGTACTGCCATGGAATAAG | | |
| | | CCCAAATAAATTTGTTAAGTACTGCATGGTTATGCAGCCAGATTGTT | | |
| | | TTAAAATGTAAACCTTAAAGTCCCCAAGGCAGAAC | | |
| prey92992 | 806 | TACATCCTTTAACTATTTATCTATAGTGCTATTAATAACAAAAAACT | 807 | YIL*LFIYSAINNKKLKGGKMLNK |
| | | GAAGGGGGGAAAAATGCTCAATAAGAGGCATTTATTAAGCAAATTAC | | RHLLSKLHFQNGIICSY*MPFKEH |
| | | ATTTTCAGAATGGGATAATATGCAGCTATTAAATGCCATTCAAAGAG | | MKYLPDTLCIEYLKSYTRNC*Q*F |
| | | CATATGAAATATCTTCCAGATACTTTATGCATAGAGTATTTGAAGAG | | LWDRALGTVVDSLPPINPLKFLTI |
| | | CTACACAAGAAACTGCTAACAGTGATTCCTTTGGGACAGAGCCCTGG | | YVHGLPLYKNKTYKSVSK*AYK*K |
| | | GGACTGTGGTAGACTCACTTCCCCCTATAAATCCTTTGAAATTTTTA | | EKKEGKEKMKKRMEKWEKVILCL* |
| | | ACCATTTATGTGCATGGATTACCTCTGTACAAAAATAAAACTTATAA | | VKFNNKVKNYSNSSVSSKLGKHWI |
| | | GTCTGTAAGTAAATAAGCATATAAATAGAAAGAAAAGAAAGAGGGGA | | LVKEGVLSFA |
| | | AGGAAAAAATGAAAAAAAGGATGGAAAAGTGGGAAAAAGTGATACTT | | |
| | | TGCTTATAGGTCAAATTTAATAATAAAGTGAAGAACTATTCAAATTC | | |
| | | TTCGGTTTCATCAAAATTAGGTAAACACTGGATATTGGTCAAAGAAG | | |
| | | GGGTCCTTAGTTTTGCA | | |
| prey92993 | 808 | ATTAGATATAGCATTTATCTTGAGATATCTGATATTTAAGTCTTTGT | 809 | IRYSIYLEISDI*VFVFF*RPGLP |
| | | ATTTTTTTAGAGACCAGGTCTTCCTCAGGCTCGAGTATNGTGATGTG | | QAGVX*CDHSLVQP*TVGFW*SFC |
| | | ATCATAGCTTAGTACAGCCTTCAACTGTTGGGTTCTGGTGATCCTTC | | LKFPSTWDYRCELLCPATRT*VLX |
| | | TGCCTTAAATTTCCAAGTACCTGGGATTACAGATGTGAGCTACTATG | | NLRSLX*FFFTHXAIR*XXMNFYI |
| | | CCCAGCTACAAGAACTTGAGTCTTANGAAATTTGAGAAGTCTANGTT | | *FSXXXDLFS*NK*TXAEKAXXXS |
| | | AATTTTTTTTCACCCACANAGCAATTAGGTAGNCANGTATGAATTTT | | GAGSXLXXPXTL |
| | | TATATCTAATTTTCANTNATNANAGATTTGTTTTCTTAAAATAAATG | | |
| | | AACTNATGCNGAGAAGGCTNNTNCCNCCTCNGGGGCAGGCTCTGANC | | |
| | | TTNANNTACCATTNACNTTG | | |
| prey92996 | 810 | GGAGGAAGAAAGGCTACTGCGAGTGCTGTCAGGAGGCCTTCGAGGAG | 811 | RKKGYCECCQEAFEELHVHLQSAQ |
| | | CTCCATGTGCATCTTCAGAGTGCCCAGCACCGGAGCTTTGCCCTGGA | | HRSFALEAHLYAEVDRTIAQLSHS |
| | | AGCCCATCTATATGCAGAAGTGGACAGGATCATTGCTCAGCTCAGCC | | FADIPFQADLPRWSGSPASDCDPL |
| | | ACAGCTTTGCAGACATCCCTTTCCAGGCTGACCTCCCCAGGTGGTCA | | CPETLHPHQPSHPRAASPRIRKED |
| | | GGTTCCCCAGCTTCTGATTGTGACCCTCTCTGTCCTGAGACTCTGCA | | SCQASGIPEQDGTVDSTQAPAERA |
| | | CCCCCATCAGCCCTCCCATCCCAGGGCAGCATCTCCCAGGATAAGGA | | GTGEVPCPIASCQDLGVSVDVFVD |
| | | AAGAAGACAGCTGCCAGGCATCAGGTATCCCAGAGCAGGATGGGACA | | PPGIPVSRSPACQCLLPSSGFMEL |
| | | GTGGACAGCACGCAGGCACCAGCTGAACGTGCAGGGACTGGTGAGGT | | SSGPDLALFGHKRK |
| | | ACCTGGACCTATAGCAAGCTGCCAGGACCTGGGGGTGTCAGTTGATG | | |
| | | TGTTTGTGGACCCCCCAGGGATACCAGTGTCCAGGAGCCCTGCTTGC | | |
| | | CAATGCCTCCTGCCTAGCTCTGGTTTTATGGAACTCTCCTCTGGCCC | | |
| | | AGACCTGGCACTTTTTCGCCACAAGCGGAAG | | |
| prey93001 | 812 | TCTTGTCATGTTGACCAGGCTGGTCTCAATCTTCTGGTCTCAGGCCA | 813 | SCHVDQAGLNLLVSGHPSTSFSPR |
| | | TCCTTCCACCTCATTCTCCCCAAGAACTGGGATTACAGGTATGAGCA | | TGITGMSNCTWSICFL*LKK*RVN |
| | | ACTGCACCTGGTCCATATGCTTCTTATAGTTGAAGAAGTGAAGGGTC | | DFTKILLK*ADLAPIIHP*SPILS |
| | | AATGACTTTACTAAAATACTATTAAAGTAATAAAGCTAGGACTTAGC | | IY*VALYYMNSKYLFYLLLSNLET |
| | | CCCAATTATTCATCCTTAAAGTCCAATACTTTCAATATATTAAGTTG | | PYKCIILYMPDRYPL*SSIPGRKT |
| | | CTCTTTATTATATGAATTCTAAATATCTTTTTTACCTTTTGTTATCT | | SSFXSLNIYSGVYYIYKGSGGGPX |
| | | AATCTGGAAATCCCATATAAATGTATAATTTTATACATGCCTGACCG | | |
| | | ATATCCTCTCTAGTCTTCTATACCAGGACGTAAGACCTCATCTTTTN | | |
| | | ATTCACTGAACATCTACTCAGGGGTCTACTATATATACAAGGGTTCT | | |
| | | GGTGGNGGGCCCANT | | |
| prey93002 | 814 | GGGGGGNCNNCATACNTNATGGCTAAAAACCGCGCACCTNACATACN | 815 | GGXXYXMAKNRAPXIXXKDFXPQX |
| | | ACNAAAAGATTTTTNACCTCAAANAAAGTTTGGATCTTTTGGATTTN | | KFGSFGFXFXFXXXIX*ICVNIYX |
| | | TTTTTTNNTTTNAGNACTNCATANNNTGAATTTGCGTTAACATTTAC | | XXTGKXGFXVFXXXXVQIXXEPGN |
| | | NGANCNNCCACTGGTAAGNNTGGGTTTNTTGTTTTTNNNTNGGANTN | | IKXXTXXXCSQSXQXXXWVPSEXX |
| | | NGTNCAAATTNCTCNGGAGCCAGGNAACATTAACGANNGAACNGANT | | TGKCXXTXXXLISRPLGXXXXXSX |
| | | GNNCATGTTCTCAGAGCTNGCAAANNANCANGTGGGTGCCTTCTGAG | | MXIAXIRAPXXAXLF |
| | | CANCANACCGGGAAATGTNTTGNGACAANGGNANCNCTNATTTCNCG | | |
| | | NCCACTGGGGNGGNGANGCANCANCTCACNCATGANNATTGCNAANA | | |
| | | TACGGGCCCCAANNNCTGCNNNTTTATTTT | | |
| prey93003 | 816 | NTTACTTTGTATACATAGTTNGAGCATAATTTTCCTTGCCATTGAAG | 817 | XTLYT*XEHNFPCH*RQKPNXNYL |
| | | ACAAAAACCAAATATNAATTACTTGTTTCTTCAATGATCACTGCATC | | FLQ*SLHPPIHSIILLLSSLLLHV |
| | | CTCCCATACACTCAATAATACTCCTACTGTCCTCCCTGTTGCTTCAT | | HFKLFGKRLRFLFVRFTVLTIFPN |
| | | GTACATTTCAAACTTTTTCGCAAAAGGCTGAGGTTTTTATTTGTTCC | | SQLIFSFRTLTLLLHNPKIYPLXP |
| | | ATTTACTGTTCTAACAATTTTTCCTAATTCTCAGCTCATTTTCAGTT | | SLRH*XF*KTIISPISFLIXLPNT |
| | | TTAGAACCTTGACACTACTCTTACATAACCCCAAAATTTACCCACTG | | GXKV |
| | | NGNCCTTCTCTTCGACATTAANATTTTTAAAAAACTATTATATCCCC | | |
| | | TATTTCTTTCTTGATATTNCTTCCCAACACTGGCNANAAAGTATA | | |
| prey93012 | 818 | TTTTTGGGATCGTTTTGATAACAAAATCAATTTAGTTTCTATACGAG | 819 | FLCWF**QNQFSFYTRYCAIXIFC |
| | | ATATGGGGCTATTCANATTTTCTCTTTATATTGTGTCAATTTTGGTC | | LYCVNFGLFHLVSSLGKLVHFT*L |
| | | TGTTTCATCTTGTATCAAGTTTAGGGAAATTGGTTCATTTTACTTAA | | IGRSL*DFLAILLMSXGXVVISSS |
| | | TTGATTGGCAGGAGTTTGTAAGATTTCCTTGCTATCCTTCTGATGTC | | FNFXGMGNXNFFFXXXXSXXXXXP |
| | | TGNAGGANCTGTAGTGATATCCTCTTCCTTTAATTTCCNGGGAATGG | | XXXXXHXAPXXPXXXXXPPPRG |
| | | GNAATANTAATTTTTTTTTTTTNGNAAANNGNAGTTTNNTTTNTNTN | | |
| | | CNGCCCCNNCNCNGNNAGNGCCACNGNGCCCCACNTNNNCCCNTAAN | | |
| | | NNNNNACTNNCCCCCCCCCCGCGGGGG | | |
| prey72968 | 820 | CCGGTCTTGCAAAGTCTCCTGCGATTGGAAGGACCCCTATGACCTCC | 821 | RSCKVSWDWKDPYEVGHSMAFRVH |
| | | GCCACAGCATGGCCTTCCCAGTGCATTTATTCTATAAGAACGGGCAG | | LFYKNCQPFPALHRPVGLRVHISH |
| | | CCTTTCCCTGCACATCGGCCTGTGGGACTAAGAGTTCACATCTCTCA | | VELAVEIPVTQEVLQEPNSNVVKVAFTVRKAGRYEAITVKLG |
| | | TGTCGAGCTAGCAGTGGAAATTCCAGTGACCCAGGAAGTCCTTCAGC | | GLNVAYSPYYKIFQPGMVVPSK |
| | | AGCCCAATTCCAACGTAGTAAAAGTGGCCTTCACTGTGCGCAAGGCT | | TKIVCHFSTLVLTCGQP |
| | | GGGCGTTATGAAATCACAGTGAAGCTTGCTGGATTAAATGTGGCATA | | |
| | | TAGTCCCTACTACAAAATTTTTCAACCTGGAATGGTGGTTCCTTCTA | | |
| | | AGACCAAAATTGTGTGCCACTTTTCTACTCTTGTATTGACCTGTGGG | | |
| | | CAGCCG | | |
| prey32308 | 822 | ATGTACGAGCAACTCAAGGGCGAGTGGAACCGTAAAAGCCCCAATCT | 823 | MYEQLKGEWNRKSPNLSKCGEELG |
| | | TAGCAAGTGCGGGGAAGAGCTGGGTCGACTCAAGCTAGTTCTTCTGG | | RLKLVLLELNFLPTTGTKLTKQQL |
| | | AGCTCAACTTCTTGCCAACCACAGGGACCAAGCTGACCAAACAGCAG | | ILARDILEIGAQWSILRKDIPSFE |
| | | CTAATTCTGGCCCGTGACATACTGGAGATCGGGGCCCAATGGAGCAT | | RYMAQLKCYYFDYKEQLPESAYMH |
| | | CCTACGCAAGGACATCCCCTCCTTCGAGCGCTACATGGCCCAGCTCA | | QLLGLNLLFLLSQNRVAEFHTELE |
| | | AATGCTACTACTTTGATTACAAGGAGCAGCTCCCCGAGTCAGCCTAT | | RLPAKDIQTNVYIKHPVSLEQYLM |
| | | ATGCACCAGCTCTTGGGCCTCAACCTCCTCTTCCTGCTGTCCCAGAA | | EGSYNKVFLAKGNIPAESYTFFID |
| | | CCGGGTGGCTCAGTTCCACACGGAGTTGGAGCGGCTGCCTGCCAAGG | | ILLDTIRDEIAGCIEKAYEKILFT |
| | | ACATACAGACCAATGTCTACATCAAGCACCCAGTGTCCCTGGAGCAA | | EATRILEENTPKKMTDYAKKRGWV |
| | | TACCTGATGGAGGGCAGCTACAACAAAGTGTTCCTGGCCAAGGGTAA | | LGPN |
| | | CATCCCCGCCGAGAGCTACACCTTCTTCATTGACATCCTGCTCGACA | | |
| | | CTATCAGGGATGAGATCGCTGGGTGCATCGAGAACGCCTACGAGAAA | | |
| | | ATCCTTTTCACTGAGGCCACCCGGATCCTCTTCTTCAACACACCCAA | | |
| | | AAAGATGACAGACTACGCCAAGAAGCGAGGGTGGGTCCTGGGCCCCA | | |
| | | AC | | |
| prey78905 | 824 | CCAGAGAGCAGAGTTTTCTCATTACAGCAGCCCCCACCTGGTGAAGG | 825 | PESRVFSLQQPPPGEGTWEPEHTG |
| | | CACCTGGGAACCAGAACACACAGGAGATTTCCACATGGAAGAGGCAC | | DFHMEEALDWPGVYLLPGQVSGVA |
| | | TGGATTGGCCTGGAGTATACTTGTTACCAGGCCAGGTTTCTGGGGTG | | LDPKNNLVIFHRGDHVWDGNSFDS |
| | | GCTCTAGACCCTAAGAATAACCTGGTGATTTTCCACAGAGGTGACCA | | KFVYQQIGLGPIEEDTILVIDPNN |
| | | TGTCTGGGATGGAAACTCGTTTGACAGCAAGTTTGTTTACCAGCAAA | | AAVLQSSEKNLFYLPHGLSIDKDG |
| | | TAGGACTCGGACCAATTGAAGAAGACACTATTCTTGTCATAGATCCA | | NYWVTDVALHQVFKLDPNNKEG |
| | | AATAATGCTGCAGTACTCCAGTCCAGTGAAAAAAATCTGTTTTACTT | | |
| | | GCCACATGGCTTGAGTATAGATAAAGATGGGAATTATTGGGTCACAG | | |
| | | ACGTGGCTCTCCATCAGGTGTTCAAACTGGATCCAAACAATAAAGAA | | |
| | | GGC | | |
| prey93040 | 826 | TTGCTGGGGTGGTTTTAATTTTATTCTGGGAATTTCAGTCATTCAAC | 827 | LLGWF*FYSGNFSHSTIKR*KVSV |
| | | CATAAAAAGGTGAAAAGTCTCTGTCAGATGTGCGTGTAGTGGTCTGT | | RCACSGLYNKD*RKQPNQLRDFL* |
| | | ATAATAAAGACTGAACGAAGCAACCAAACCAATTAAGAGACTTTCTC | | RQSNAKGSGSDKSGGKSL*SFGRQ |
| | | TAAAGGCAGAGTAATGCTAAGGGCAGTGGCAGTGACAAAAGTGGAGG | | NRKCLVMRCEARRGEVEGKQSCSG |
| | | AAAAAGTCTGTGATCATTTGGGAGACAGAATAGGAAGTGCTTGGTCA | | SAGEDWVCS |
| | | TGAGATGTGAAAGAAGAGGGGAAGTAGAAGGAAAACAGAGCTGCTCA | | |
| | | GGCTCTGCTGGGGAAGACTGGGTATGTAGCAG | | |
| prey93042 | 828 | GACACTCCTGTGCCTGAGGGAAAGGCAATGCCTGCGGCAGAAACGGA | 829 | DTPVPEGKANPAAETERSPSQGRE |
| | | GCGGTCCCCCAGCCAGGGGAGAGAGTGGACCTAGTATGGTACCCAAT | | WT*YGTQCSKAWS*LTADCRPATH |
| | | GCTCTAAGGCATGGAGCTGATTGACTGCTGACTGTAGACCAGCCACT | | HSVGWKPSKAVRSAEVSP*WGPGV |
| | | CACCACAGTGTAGGTTGGAAGCCTTCTAAGGCCGTGCCTTCTGCAGA | | WAXSVTPLGI*SWLHLWGPWVXFL |
| | | AGTCAGCCCATGATGGGGGCCAGGAGTGTGGGCTNCCAGTGTCACCC | | *LLYASVSSSVWEQ*CLLHRVVGK |
| | | CCCTTGGGATTTAATCCTGGCTGCACCTTTGGGGGCCTTGGGTAANG | | ELSTIIPVELLEEWLAERKHSVXA |
| | | TTCTTGTAACTCCTCTATGCCTCAGTTTCCTCATCTGTATGGGAACA | | VMTXGSPDKMQAKDCRAWRETPDG |
| | | ATAATGCCTACTTCATAGAGTTGTGGGGAAAGAACTAAGCACAATAA | | S |
| | | TCCCTGTAGAGCTCTTAGAAGAGTGGCTGGCAGAGAGGAAGCACTCA | | |
| | | GTANAGGCTGTGATGACACNCGGCAGCCCTGATAAGATGCAATAGAA | | |
| | | AGATTGCAGAGCTTGGAGAGAAACACCGGATGGGTCCC | | |
| prey93049 | 830 | TTCAGAGATGCCCTGCCCAGTGAGGAGGAATCTAGAGAAGCAGTCTG | 831 | FRDALPSEEESREAVWPQLLCCAA |
| | | GCCACAACTGCTTTGCTGTGCTGCGGTCAATTCTTCCCAGTCCAAAC | | VNSSQSKPPSLLSTVKVQQPPEDS |
| | | CTCCCAGTCTCCTTAGCACTGTCAAGGTACAACAGCCTCCTGAAGAC | | VMADAHQARSSHVDSRLLCWL*EF |
| | | TCAGTAATGGCGGATGCCCACCAAGCTCGGTCATCCCACGTTGACTC | | QASGS*LAGLRDSGTY*VRPLGSL |
| | | CAGACTGCTGTGCTGGCTGTGAGAATTTCAAGCCAGTGGTTCTTAGC | | ALAPFPGE*TVLSYWGSRCH*GTK |
| | | TTGCTGGGCTCCGTGACAGTGGGACCTACTGAGTGAGACCACTTGGC | | KKTTXSSGACPKGPPSFXGLKPRA |
| | | TCCCTGGCATTAGCCCCCTTTCCAGGGGAGTAAACGGTTCTGTCTTA | | P |
| | | CTGGGGTTCCAGGTGCCACTGAGGTACAAAAAAAAAAACTACAGNTA | | |
| | | GCTCGGGGGCCTGCCCNAAGGGNCCNCCCAGTTTTTGNGGTTTGAAA | | |
| | | CCCAGGGCCCCT | | |
| prey93052 | 832 | AAGTCTGAATTCCAGTATCTGGGATGGATCTGAAGTTCTGAGGCGAA | 833 | KSEFQYLGWI*SSEANPRTTACSG |
| | | TCCGAGGACCACTGCTTGCTCAGGTATTAAATATTTTCTCTTTACAT | | IKYFLFT*NFIIFQKF*VPVVILS |
| | | AAAATTTTATAATATTCCAAAAATTTTAAGTTCCTGTCGTCATCCTT | | LLKGFIGLSEARILMSTTASST*L |
| | | TCCTTACTGAAGGGGTTTATAGGTCTTTCATTTGCTAGAATTATGTC | | CLSLASRVKVAREPLEFKFVSV*L |
| | | GACTACTGCATCCAGCACTTAGCTATGCCTTTCTTTAGCCTCCAGGG | | *FESLKKSYLSPG |
| | | TAAAGGTTGCAAGGGAGCCATTGGAATTTAAATTTGTTAGTGTATGA | | |
| | | TTGTGATTTGAATCACTTAAAAAGTCATATCTGAGCCCAGGCC | | |
| prey93055 | 834 | GAAGAACTCTNGTCCTTTTTTTCAGNAGGATGGNGTTAACACATTCC | 835 | EELXSFFSXGWX*QIPLSPLFLAX |
| | | TCTGTCACCTTTATTTCTCGCTANNTATACATTTAGATCTCGAGGCC | | YTFRSRGLVRFRFNYLFNXQENF* |
| | | TAGTTAGGTTCAGATTTAATTACTTATTTAATTNTTAGCAAGAAAAC | | VGACIPSXRXX*XFMSLYXITXH* |
| | | TTTTAGGTGGGGGCATGTATACTTCCNTCAGNTAGATNCNCATAANG | | XPLSRSIIXLGTYLVVELSDEXGK |
| | | TTTTATGTCTCTCTATNATATTACCAGNCACTGATNACCATTGTCTA | | IFHEMXIIXTXXXTYYFX*GXTXX |
| | | GATCTATTATTNTACTAGGGACATACCTGGTTGTTGAATTATCCGAT | | XLYYXHGKMGKKIXSGTXFXXPXX |
| | | GAAAANGGAAAAATATTCCATGAAATCGNGATCATTGNAACCNTTGN | | X |
| | | CTANACCTATTATTTTGNCTAGGGACNTACTCNCNCTGNNTTGTATT | | |
| | | ATNCCCATGGAAAAATGGGAAAGAAAATCNACTCTGGAACNTNATTC | | |
| | | NTTNCNCCCANANAANNANC | | |
| prey93057 | 836 | CTTTGTCCTCAATGGCATTGTCTAGGATTCTTCTAGGATTTTTATGG | 837 | LCPQWHCLGFF*DFYGFRLTFKSL |
| | | TTTTAGGTTAACATTTAAGTCTTTAATCCATTTTGAATTAATTTTTG | | IHFELIFVAGVRKGSSFSFLHMAS |
| | | TGTAAGGTGTAAGGAAGGGATCCAGTTTCAGCTTTCTACATATGGCT | | QFSYHCLLNRESFSHFLFLSGLSN |
| | | AGCCAGTTTTCCTACCACTGTTTGTTAAATAGGGAATCTTTTTCCCA | | IRWL*MCVLFLRPLFCSIGLYICX |
| | | TTTCTTGTTTTTGTCAGGTTTGTCAAACATTACATGGTTGTAGATGT | | VPGPCHFGYWRIXL*Y*RPXKXDA |
| | | GTGTGTTATTTCTGAGGCCTCTGTTCTGTTCCATTGGNCTATATATC | | XHPXXF |
| | | TGCTNNGTACCAGGACCATGCCATTTTGGTTACTGGAGGATTGNACT | | |
| | | ATAGTATTGAAGGCCANGGAAGNGTGATGCCNTCCACCCTTNATTNT | | |
| | | TTT | | |
| prey93069 | 838 | TCCCTGNNANCGTCCCGAACCATGNGNCATGAANNGACCTGAGCGNT | 839 | SLXXSRTMXHEXT*AXXKXFXD*X |
| | | NAANAAGGNCTTTCNTGATTGAANGNTNATCGTNNACCTAGNAAANC | | XIVXLXXXDXEDXXDVXXXXXGVV |
| | | NGGACTNTGAGGACCANATNGACGTNANGNACTNTNTGNATGGNGTG | | LXDXXXXXQRXXYXTLTXSLPLLP |
| | | GTCCTGANNGATNCTNATNATNTCNNACAGCGGNAAGNCTATTTNAC | | XRTWMSWSXXXXWTXXXPTYRKIP |
| | | NCTGACCNGNTCCTTACCGCTATTGCCNNGCAGGACCTGGATGTCCT | | PPPEHDIPLATKYTEXLIMXXXRX |
| | | GCTCCTNANNTTTNNCATCGACCNNNTNGNNNCCAACGTACAGGAAG | | TPYPXQSLKFLQTFPVXXTLQX |
| | | ATCCCACCNCCCCCAGAGCACGACATTCCNCTTGCTACGAAGTACAC | | |
| | | TGAACNCCTAATCATGAANTNTCNGCGANGCACNCCTTACCCCTTNC | | |
| | | AATCTTTAAAATTCCTNCAAACCTTCCCTGTGTNTNCNACACTACAA | | |
| | | NNNCC | | |
| prey93071 | 840 | NTCTNTCTTNAGTGATTCTCCTACCTCATTCTCCCAAAATGTANGGA | 841 | XXLX*FSYLILPKCXDGRNXPPQL |
| | | TCGCAGGAATCNCCCACCACAACTGGTCTCTTTTAGCTGTTTTGTAA | | VSFSCFVI*DNFSLIIIILLCNKK |
| | | TATAAGATAACTTTTCATTAATTATTATTATTCTCCTGTGTAACAAA | | QKLISPI*L*HNTFEAAFSHLPAS |
| | | AAACAAAAACTTATTTCTCCTATCTGATTGTAACACAATACTTTTCA | | VSGNPCXTLCLYQPFFXGSSNESD |
| | | AGCTGCCTTTTCCCATCTCCCTGCTTCAGTCTCTGGTAACCCCTGTN | | XXVXXD*XXSLAAYXT*HGXPQGS |
| | | GTACTCTTTGCTTATATCAACCCTTTTTTTNAGGTTCCTCAAATGAG | | SLLF*LXEFLSFSYXAIXXPWCPX |
| | | TCACATNATANGGTCANAANGGATTAGTGNTNCTCTCTNGCGGCTTA | | GX |
| | | CTTNACTTAACACGGGANGCCTCAAGGTTCATCCCTGCTCTTTTAAC | | |
| | | TNANNCAATTTTTATCCTTTTCTTATNGCTAAATANGGTNNCCCTGG | | |
| | | TGCCCATNTCCCNCC | | |
| prey93076 | 842 | CCCCAGAGCCTGCCAGCCAGGCCCGAGTCCTCTCCAGCTCANAGACC | 843 | PQSLPARPESSPAXRPLPGPCPSP |
| | | CCTGCCAGGACCCTGCCCTTCACCACAGGTGAGACCGGGAGGACGGT | | QVRPGGGWRVEGGARHARVHVGVL |
| | | GGCGGGTGGAGGGAGGGGCTCGGCATCCACGAGTCCATGTGGGTGTC | | VSLGTFLEATECLXLLGPRXTHGA |
| | | TTGGTGTCACTTGGGACATTTTTAGAGGCCACAGAGTGCTTANCCTT | | HGTAXYCIXPMRTKAXXI*GDKPV |
| | | GTTAGGCCCACGTANTACCCATGGAGCACATGGAACAGCTNGTTATT | | VK*QVVVKPG*XFXXPANALNPFK |
| | | GCATTNGGCCGATGAGGACCAAGGCTNACNAAATCTAAGGTGACAAG | | KMIFITVCSSLX |
| | | CCTGTGGTCAAATAGCAAGTGGTAGTCAAGCCAGGATAANAATTCCI | | |
| | | ANNCTCCTGCCAATGCACTCAACCCTTTTAAAAAAATGATATTTATT | | |
| | | ACTGTCTGCTCTTCCTTGNAAC | | |
| prey93082 | 844 | GAGGAAGAGGTAGGGTGGGAACCCGGGTTGTCCCTCTTTCCTGTCCC | 845 | EEEVGWEPGLSLFPVPTSPCCLPR |
| | | CACCAGCCCATGCTGCCTCCCCAGGACTGTGGCACATGTGGGTGAAG | | TVAHVGEEHTLQSDYGSSSPGSWT |
| | | AGCACACTCTGCAGTCAGACTACGGGAGTTCAAGTCCTGGATCCTGG | | LLAVI*LSLPKCGRQYW*LPQRLM |
| | | ACACTGCTTGCCCTGATTTACCTTTCTCTGCCAAAATGTGGGAGGCA | | VIIRGNDMCNTRSPVPGK*SCLLN |
| | | ATACTGGTAGCTACCACAGAGGCTTATGGTGATAATTAGAGGAAATG | | VSGDDGNDENSC*RLLSRPEVRLL |
| | | ACATGTGCAACACACGCAGCCCAGTGCCTGGCAAATAGTCCTGCCTG | | V*SLG*DDYIALS*KRDTFS |
| | | CTAAATGTCAGTGGTGATGATGGTAATGATGAAAATTCGTGTTAAAG | | |
| | | ACTGTTGTCCAGGCCAGAGGTGAGGCTCCTAGTCTAGTCATTAGGGT | | |
| | | GAGATGATTATATTGCTCTTAGCTAAAAAAGGGACACATTTTCTAT | | |
| prey93091 | 846 | GGAATAGATGCTAATTCAGGCTCCACAGATAGTTCTGGTGATGGGGT | 847 | GIDANSGSTDSSGDGVTFPFKPES |
| | | TACATTTCCATTTAAACCAGAATCCTGGAAGCCTACTGATACTGAAG | | WKPTDTEGKKQYDREFLLDFQFMP |
| | | GTAAGAAGCAGTATGACAGGGAGTTTCTGCTGGACTTCCAGTTCATG | | ACIQKPEGLPPISDVVLDKINQPK |
| | | CCTGCCTGTATACAAAAACCAGAGGGCCTGCCTCCTATCAGTGATGT | | LPMRTLDPRILPRGPDFTPAFADF |
| | | GGTTCTTGACAAGATCAACCAACCCAAATTGCCAATGCGAACTCTGG | | GRQTPGGRGVPLLNVGSRRSQPGQ |
| | | ATCCTCGAATTTTGCCTCGAGGACCAGACTTTACACCAGCCTTTGCT | | RREPRKIITVSVK |
| | | GATTTTGGAAGGCAGACACCTGGTGGAAGAGGCGTACCTTTGTTGAA | | |
| | | TGTTGGGTCACGAAGATCTCAACCTGGCCAAAGAAGAGAACCCAGAA | | |
| | | AGATCATCACAGTTTCTGTAAAAGA | | |
| prey93100 | 848 | GGATGGCCCTGCCCTCTTTGTTCTCAGGAGTCTTCCAGGAAGTTCCA | 849 | GWPCPLCSQESSRKFQSQRLFISI |
| | | GTCACAACGTTTGTTTATATCTCACTGGCCAGAACTTAAGTCATATG | | AIWPELKSYGHL*LQGRLGDVVCN |
| | | GCCACCTCTAGCTACAAGGAAGGCTGGGAGATGTAGTCTGTAACTGG | | WMAILSTKNY*K*QEE*IMEGNLL |
| | | ATGGCCATATTATCAACTAAAAATTACTGAAAATGACAAGAAGAATA | | FLSKDDFKRESCKSISSLTVS*QF |
| | | AATAATGGAAGGCAACCTGTTGTTTCTGTCAAAGGATGACTTCAAAA | | SSEVTLFICDASKF*YLFSSSPIC |
| | | GAGAAAGTTGCAAAAGTATTTCAAGCTTGACAGTATCATGACAGTTT | | SEIGTLGGN*WVCKPVQTF*RTV* |
| | | TCTTCAGAGGTGACGCTATTCATTTGCATGTCCAAGTTCTGATATCT | | PNXMNSGN*S**NVKGAHEDCFAD |
| | | GTTTTCAAGTTCACCTATTTGCAGCGAAATAGGAACTCTTGGAGGCA | | CNNKK*RKYFIIGDRCNX*YIPQX |
| | | ACTAGTGGGTGTGTAAACCGGTACAAACTTTCTAGAGGACAGTTTAA | | SVIMLLNVCGIHNLLCQKKALRMA |
| | | CCCAACNAGATGAATTCTGGAAATTGATCCTAATGAAATGTTAAGGC | | CLFCRYLXX*VHAHT*ER |
| | | TGCTCATGAAGATTGCTTTGCCGATTGCAATAATAAAAAGTAAAGAA | | |
| | | AATATTTTATAATTGGGGACAGGTGTACTTNTGATACATTCCACAAN | | |
| | | GGAGTGTAATAATGCTGTTAAATGTTTGTGGAATTCACAATCTTTTA | | |
| | | TGCCAAAAGAAAGCNTTAAGAATGGCTTGTCTTTTTTGTAGATACTT | | |
| | | GTANTNATAAGTCCATGCACATACATAAGAAAGGAN | | |
| prey93103 | 850 | TTGGAAACTGGAGGTGAGCTAACAAGAAGCAGGATGTTTTCTGTGAC | 851 | LETGGELTRSRMFSVTGLGSAFLG |
| | | TGGTTTGGGGAGCGCTTTCTTGGGTTGGTTCTGACATGGGGAGGGTG | | WF*HGEGGTKNRGASNPDHVLPXX |
| | | GTACAAAAAATAGAGGAGCAAGCAATCCTGACCACGTCCTTCCCAAN | | PTARLLQRRAVS*LSFVISPLSIX |
| | | CANCCGACTGCCCGGTTACTGCAGAGGCGTGCAGTCAGTTGATTGTC | | ILXLTFFFXLLTVPKXEKFXNYHY |
| | | ATTCGTTATCTCACCATTGTCTATTGNCATATTGNCTCTCACTTTTT | | LPPXPXTRXXVLQXLXPPXRXPXX |
| | | TTTTCTNACTACTTACTGTACCAAAGTTNGAAAAATTTAANAATTAC | | XSIDIVWXVXLAGXXWX |
| | | CATTACCTACCACCTTANCCACANACACGTCNNTNGGTNCTCCAGAN | | |
| | | GCTGNGNCCNCCCNCACGGANCCCCNAACNATNNAGTGACATTGTCT | | |
| | | GGCNTGTGCNCCTNGCGGGGCNNTNTTGGNGG | | |
| prey93104 | 852 | AATTCTACCCATTATTCCAAGGTACAGCTCAAATACTACCCCGTTTT | 853 | NSTHYSKVQLKYYPVL*NFP*FSQ |
| | | ATAAAACTTTCCTTAATTTTCTCAGTTAAACGTCTTATATCCTGCCT | | LNVLYPASEVPLTS*ESLLSYLID |
| | | CTGAAGTTCCCTTAACGTCATAGGAGTCATTATTATCTTATCTCATT | | *ITSLD*HFSSL*AHGSFTLLLIG |
| | | GACTAGATTACAAGCCTTGACTAACATTTTTCATCCTTGTGACCACA | | TQQMFA**MYFVRLEDLIYILTSL |
| | | TGGCTCTTTTACCTTGCTCCTAATAGGTACTCAACAAATGTTTGCTT | | LS*KIHLWKCIHFQFFIWGCRLSN |
| | | AATAAATGTATTTTGTAAGACTCGAAGACTTAATATATATACTGACA | | TSLPPGTSSLPRDRVAEDTAIIII |
| | | TCATTGCTCTCTTAAAAAATACATTTATGGAAATGTATCCATTTCCA | | SRDKGEESLRE*SPKVLRNPGFTA |
| | | ATTTTTCATTTGGGGATGCAGGCTTTCCAATACATCCCTTCCCCCTG | | FSP |
| | | GAACCAGCAGTCTACCCAGAGATAGAGTTGCTGAGGACACTGCAATT | | |
| | | ATCATCATTTCTAGAGACAAAGGTGAAGAAAGTCTAAGGGAATAAAG | | |
| | | CCCAAAGGTACTGAGAAATCCGGGTTTTACTGCTTTCTCTCCC | | |
| prey93105 | 854 | AAGGATGGCAACNTGGGGAAGGAAGTGANAGGAGGAGCCNCCCTCTT | 855 | KDGNXGKEVXGGAXLFSVGWHKKG |
| | | TTCCGTTGGGTGGCATAAAAAAGGATTGANTNCCCCCTTNTNTGCCN | | LXXPXXAXAFGLXPFXPGKGQGPY |
| | | AGGCCTTTGGCTTGGNCCCCTTTTANCCTGGGAAGGGGCAGGGGCCT | | LXXFXX*XKLGXXXXKXXXFWXXX |
| | | TACCTTTANNTNTTTTNNCNTTGAANNAAATTAGGGGNTNGGGNAAN | | FXFXSFGXLKGGXXXGXPKXFXPP |
| | | AAAAAANCNNTTNTTTTTGGANNANCNNTTTTTTNTTTNAAAGCTTT | | XX |
| | | GGGCNTTTAAAGGGGGGGGNNNNGAANGGGGNCCCAAAANNTTTTTT | | |
| | | NCCCCCCTNAANAG | | |
| prey93118 | 856 | TGCTTCNTCTCATTCNGACNTGNTGNATTAAATATTTCANCTTCNGT | 857 | CLXSFXXXXLNISXSVXKKGXXIL |
| | | TATNAAGAAAGGTCNNTNTATACTATTCCACCTGTCATCCTAAATTA | | FHLSS*IMHXIFXYLTHLTSLLPA |
| | | TGCACANAATATTCNAATATCTTACTCATTTAACTTCATTGCTTCCA | | VQTRAR*GNQS*R*NCHQRRSSSY |
| | | GCCGTTCAAACCAGGGCCAGATGAGGTAATCAATCATAGAGATAGAA | | *SEPEH*TA*KYFLLVHVVRLTRN |
| | | TTGCCACCAAAGAAGGTCGTCTTCTTATTAGTCAGAACCTGAACATT | | AEGK*VEQVREAVFTPMYPSWVFF |
| | | AAACAGCATAAAAGTATTTCTTACTTGTGCATGTAGTAAGACTCACT | | *SKSLIKL |
| | | AGAAATGCTGAAGGGAAATAAGTAGAGCAGGTGAGAGAAGCAGTTTT | | |
| | | TACTCCGATGTATCCAAGTTGGGTTTTTTTCTGATCAAAAAGCTTAA | | |
| | | TTAAGCTTT | | |
| prey93122 | 858 | TCCTTGGCTTTCTTCTTCAGGTGTTCACCATGTCCCAGTAGAGCTGA | 859 | SLAFFFRCSPCPSRAEPHVSHIGA |
| | | GCCACACGTATCACACATAGGTGCCACTGTGTTAGCCATTATTTAGC | | TVLAII*PCMKNPYIFSLHQISKF |
| | | CATGCATGAAAAATCCTTACATCTTCAGCCTTCATCAAATCTCTAAG | | NMFFIMLS*NKGYLFCSEV*HKCR |
| | | TTTAATATGTTCTTTATCATGCTTTCTTAAAACAAGGGATACCTGTT | | DFPGIFPSQWLEASPILNGETTLF |
| | | TTGCAGTGAAGTCTGACACAAATGCAGAGATTTCCCAGGAATTTTTC | | TIVKTKKVLKDRHYCCFPF*EVME |
| | | CTAGTCAGTGGCTTGAAAGTCCAATTCTAAATGGGGAGACAACATTA | | TPCGFTYI*TYIHT |
| | | TTTACAATTGTAAAAACCAAAAAAGTTCTCAAGGACAGGCATTATTG | | |
| | | TTGCTTTCCATTTTGAGAGGTCATGCAAATCCCATGTGGATTTACGT | | |
| | | ATATATAAACATATATACATACAGA | | |
| prey37079 | 860 | ATGCTATCACATAATACTATGATGAAGCAGAGAAAACAGCAAGCAAC | 861 | MLSHNTMMKQRKQQATAIMKEVHG |
| | | AGCCATCATGAAGGAAGTCCATGGAAATGATGTTGATGGCATGGACC | | NDVDGMDLGKKVSIPRDIMLEEAL |
| | | TGGGCAAAAAGGTCAGCATCCCCAGAGACATCATGTTGGAAGAATTA | | SHLSNRGARLFKMRQRRSDKYTFE |
| | | TCCCATCTCAGTAACCGTGGTGCCAGGCTATTTAAGATGCGTCAAAG | | NFQYQSRAQINHSIAMQNGKVDGS |
| | | AAGATCTGACAAATACACATTTGAAAATTTCCAGTATCAATCTAGAG | | NLEGGSQQAPLTPPNTPDPRSPPN |
| | | CACAAATAAATCACAGTATTGCTATGCAGAATGGGAAAGTGGATGGA | | PIDNIAPGYSGPLKEIPPEKFNTT |
| | | AGTAACTTCGAAGGTGGTTCGCAGCAAGCCCCCTTGACTCCTCCCAA | | AVPKYYQSPWEQAISNDPELLEAL |
| | | CACCCCAGATCCACGAAGCCCTCCAAATCCAGACAACATTGCTCCAG | | YPKLFKPEGKAELPDYRSFNRVAT |
| | | GATATTCTGGACCACTGAAGGAAATTCCTCCTGAAAAATTCAACACC | | PFGGFEKASRMVKFKVPDFELLLL |
| | | ACAGCTGTCCCTAAGTACTATCAATCTCCCTGGGAACAAGCCATTAG | | TDPRFMSFVNPLSGRRSFNRTPKG |
| | | CAATGATCCGGAGCTTTTAGAGGCTTTATATCCTAAACTTTTCAAGC | | WISENIPIVITTEPTDDTTVPESE |
| | | CTGAAGGAAAGGCAGAACTGCCTGATTACAGGAGCTTTAACAGGGTT | | DL* |
| | | GCCACACCATTTGGAGGTTTTGAAAAAGCATCAAGAATGGTTAAATT | | |
| | | TAAAGTTCCAGATTTTGAGCTACTATTGCTAACAGATCCCAGGTTTA | | |
| | | TGTCCTTTGTCAATCCCCTTTCTGGCAGACGGTCCTTTAATAGGACT | | |
| | | CCTAAGGGATGGATATCTGAGAATATTCCTATAGTGATAACAACCGA | | |
| | | ACCTACAGATGATACCACTGTACCAGAATCAGAAGACCTATGA | | |
| prey93127 | 862 | TGCCTAGACCAACTGTTTTGGAAAAAGATGCTTACATCAACCATAAT | 863 | CLDQLFWKKMLTSTIIMHFMVFFS |
| | | TATGCATTTTATGGTATTTTTTAGCCCTCTGAAAAGGGAATCCTCCC | | PLKRESSLNL*IAKSKFVLFLHSH |
| | | TAAACCTCTAAATCTAAAAATCTAAGTTTGTTTTGTTTCTACATTCC | | FQI*SWKQIKH*GSSIAHFSSHNE |
| | | CATTTCCAAATATAATCATGGAAGCAAATTAAGCACTGAGGTTCGTC | | HIYICRNSSFNTLLGEFVFPPSLT |
| | | TATAGCTCATTTCTCTTCTCACAATGAACATATCTACATCTGCAGAA | | DCL*LWLSSLKTDKIY*S*NFSSK |
| | | ATTCATCTTTTAATACTCTTCTAGGGGAATTTGTGTTCCCACCTTCC | | L*QSQQLAPSHLHQLEVQQYRFSL |
| | | CTAACTGATTGTCTTTAGCTATGGCTATCCTCTCTCAAGACTGATAA | | MILL*LTPIPVINTWLCFKGCIFF |
| | | GATTTACTGAAGCTGAAACTTCTCATCTAAACTCTGACAATCCCAGC | | LAIDNHILKNSVLSL |
| | | AACTGGCCCCCAGCCACCTCCACCAGCTAGAGGTTCAACAATACAGA | | |
| | | TTCTCCTTAATGATCCTACTCTAGTTGACTCCTATCCCAGTAATTAA | | |
| | | TACATGGCTGTGCTTCAAAGGCTGCATTTTTTTTCTCGCTATAGATA | | |
| | | ATCATATTCTTAAAAATAGTGTTTTATCCTTAAA | | |
| prey93134 | 864 | GGGACTAGGTGCTTGTGCCGAGCTGTGGCCGCTGGGCAGGGGTGTTG | 865 | GTRCLCRAVAAGQGCCRGHTEQKA |
| | | CAGGGGCCACACAGAACAGAAAGCAAGGAGAGCAGCCCGGGGCACCG | | RRAARGTAGLKRGRGRGPPGR*ER |
| | | CGGGGCTGAAGAGGGGGAGGGGCCGTGGCCCACCTGGCAGGTGAGAG | | GTEKVAWWHR*APCGSLLKEWSHL |
| | | AGGGGCACGGAGAAGGTGGCCTGGTGGCACAGGTGAGCGCCGTGTGG | | SLLTLAVTWNPFSPKPTGEGEGLG |
| | | CAGCCTCTTGAAGGAGTGGAGCCATCTGTCCCTGCTGACACTAGCTG | | HPHFLVLRKRTSLFQGSS*PLKII |
| | | TCACCTGGAATCCTTTCTCACCCAAGCCCACAGGGGAAGGGGAAGGG | | L*SRKILMNLIKRLKPS |
| | | CTTGGGCACCCACATTTTTTGGTTCTAAGGAAGCGTACCTCGCTGTT | | |
| | | TCAGGGGAGCTCGTGACCTTTAAAAATTATCCTTTGATCTAGGAAAA | | |
| | | TCCTAATGAACTTGATCAAGCGTCTAAAGCCCTCCC | | |
| prey93137 | 866 | TTTACTTTGTATTCGTTATTATAGTAATCTAGAGATGATTTAAAGGG | 867 | FTLYSLL**SRDDLKGREDIFRLY |
| | | ACGGGAAGATATATTTAGGTTATATGTAAATATACCATTTTATATGA | | VNIPFYMRNVYILGF*NL*GFLEP |
| | | GGAACGTGTATATCCTTGGATTTTAGAATCTGTAGGGATTCCTGGAA | | VFHGYRGMTVLFCEIFVPPTYLYV |
| | | CCAGTCTTCCATGGATACCGAGGGATGACTGTATTGTTCTGTGAAAT | | CMFLVRYNCIIFLTMG*TTL*DIV |
| | | TTTTGTCCCACCTACATATTTGTATGTATGTATGTTTTTGGTCCGGT | | RNLVSSGLDHSRRPFPCAHIVFSL |
| | | ATAATTGTATAATATTTCTTACTATGGGTTAAACAACTTTATAAGAC | | KSGDCLKN* |
| | | ATTGTGAGGAACCTGGTATCCTCTGGACTAGATCATTCCCGTAGACC | | |
| | | GTTCCCTTGTGCACACATAGTATTTTCGTTAAAATCAGGTGACTGTT | | |
| | | TAAAAAATTAAAT | | |
| prey93141 | 868 | TTCAAGGAAAGATTACTGCAGGAAGTGAGTTTTGAACAAGTTTTTTG | 869 | FKERLLQEVSFEQVFCCCCFCFET |
| | | TTGTTGTTGTTTTTGTTTTGAGACAGGGTCTCACCCTGGTGCTCAGG | | GSHPGAQAGVQWHDLGSL*SPPPG |
| | | CTGGAGTGCAATGGCATGATCTTGGCTCGCTTTAATCCCCGCCGCCC | | LK*XXXXXXXXXXXXXXXXXXXXX |
| | | GGGCTCAAGTGATNNNNNNNNNNNNNNNNNNNNNNNNNNNNNNNNNN | | XXXXXXXXXXXXXXXXXXXXXXXX |
| | | NNNNNNNNNNNNNNNNNNNNNNNNNNNNNNNNNNNNNNNNNNNNNNN | | XXXXXXXXXXXXXXXXRFFFFFKK |
| | | NNNNNNNNNNNNNNNNNNNNNNNNNNNNNNNNNNNNNNNNNNNNNNN | | KPGXXXXPXXXXWAXXPPXP |
| | | NNNNNNNNNNNNNNNNNNNNNNNNNNNNNNNNNNNNNNNNNNNNNNN | | |
| | | NNNNNTCCGTTTTTTTTTTTTTTTTAAAAAAAAGCCNGGGNCCCNNN | | |
| | | GGGNCCCCNTTNNNNNANTTTGGGCTNAANNCCCCCCNCNCCCC | | |
| prey93144 | 870 | TAATCTTGACCTTTTACCCTTGCATATAAATTTTAGAGTCAGCTAAA | 871 | *S*PFTLAYKF*SQLKFCQVLQNK |
| | | ATTTTGCCAAGTTCTGCAAAACAAACTTTTGAATTTTTTTGGAATTA | | LLNFFGIIFN*QIYLKIFYTXRLL |
| | | TATTCAATTAACAGATATATTTGAAAATATTTTACACCTNCAGACTA | | IYPSIYLDILLASLIKFYNFLHKV |
| | | TTGATTTACCCATCCATTTATTTAGATATCCTTTTAGCTTCTTTAAT | | LLYLVLTYS*VRYFWCCXXWDLL* |
| | | AAAGTTTTATAACTTTCTTCATAAAGTTCTTCTATATCTCGTTTTAA | | ITFSKHFC*GTKA*XFYVTLYPGX |
| | | CTTATTCCTAAGTAAGGTACTTTTGGTGCTGTTGNNNCTGGGATCTT | | SGKLYXXVLV |
| | | CTTTAAATCACATTTTCTAAACATTTTTGCTAAGGTACAAAAGCATG | | |
| | | ATNTTTTTATGTTATCTTGTATCCGGGCANCAGTGGTAAGCTATACA | | |
| | | ANNAGGTTCTGGTG | | |
| prey93145 | 872 | GCTAAANATTTCANACNACAACANAGGNGCGNGTGTGTTTTANATAT | 873 | AKXFXXQXRXXCVLXMYREYIEHR |
| | | GTACCGAGAGTATATAGAACACAGAGTATNTTTTTATAATTTTACAT | | VXFYNFT*XYYNIETYXAYXSLTP |
| | | GACNTTATTACAATATATTTACNTATNTNGCATATTGNTCCTTAACA | | KGT*FNVILIILTFYYXLSLLHXL |
| | | CCNAAAGGAACATAATTTAATGTTATTTTAATTATAACATTTTATTA | | *IXMXAX*NKKNSXYXXXMXXLIX |
| | | TNACCTATCTTTATTACATNTTCTTTAGATATNAATGTANGCCNTTT | | XAGXFXFQISXLXXWLLPXXAX*F |
| | | AAAACAAAAAAAATTCTNATTACGNGGNTAANATGTANNATTTAATA | | WIGKXNXXPXXXXFXGPPRXPPXX |
| | | NGNTGNGCAGGTNATTTCANTTTTCAGATCTCNTTNCTCCNTGNTTG | | RXRXAXSPPP |
| | | GCTTCTNCCCTTNNCCGCTNCCTGATTTTGGATTGGTAAATTNAATN | | |
| | | NANCACCCNCTANATANTNTTTCCNTGGNCCNCCCAGGGNNCCCCCN | | |
| | | NNCAGNAGGNTCCGNTGNGCCNNCAGCCCCCCCCCNC | | |
| prey93151 | 874 | ACNGATNCTNGCTTTGATCCCANGGCTGGANNGCATNGCCACTATCT | 875 | TDXXFDPXAGXHXHYLGYVLXPDS |
| | | TGGCTACGTATTANCNCCTGACTCCCGGGTTGNAGTTATTCTCCTNC | | RVXVILLPQPPLXSXDYXXGPPKP |
| | | CGCAACCTCCTTTANTNTCNNTGGATTACCNGAGNGGGCCACCCAAG | | GXFL*LLVADGVSPXLARXLWIT* |
| | | CCCGGCNAATTTTTATAACTTTTAGTNGCAGACGGGGTTTCACCANG | | PXDXXXLXSPSAWXTGLXQRALPL |
| | | ATTAGCNCGGCNGCTNTGGATCACCTAACCTNGTGATNCANCCNCNT | | XFYL*XTXTXAMLXXPCLRLLTXC |
| | | TGNTCTCCCCAAGTGCTTGGATNACAGGCCTNAGNCAACGCGCCCTG | | XTPSSPTGV |
| | | CCTTTANTNTTTTATTTATAGNAAACACNGACTTNCGCCATGTTGNN | | |
| | | ANGGCCATGTCTCAGACTCCTCACCTNATGTGANACACCCTCCTCTC | | |
| | | CNACGGGAGTGC | | |
| prey93154 | 876 | GATGATGATAATCGTACTGACAGTAATAATAAGCTCAAATATATAGA | 877 | DDDNRTDSNNKLKYIELTMYHELF |
| | | GCTTACTATGTATCATGAATTGTTCTGAATGCTTTATAAATATATGT | | *MLYKYMLPPLPS*QPSKGAIPHF |
| | | TACCTCCTTTACCCTCATGACAGCCCAGTAAAGGCGCCATTCCCCAT | | TAGETELQSLSALSHQEQMLDQVI |
| | | TTTACAGCTGGGGAAACTGAGTTACAGAGCTTGTCTGCACTGAGTCA | | EPKQSGSRAK*MYFLCYTNIYIYF |
| | | TCAGGAGCAAATGCTAGATCAGGTAATTGAACCCAAGCAATCTGGTT | | *GKGGCRMG*XPPPPXXXLVYXXX |
| | | CCAGAGCCAAATAGATGTATTTTTTATGTTATACAAACATATACATA | | XXXTXXXRX |
| | | TATTTTTAGGGGAAGGGTGGGTGTAGGATGGGATGANGTCCNCCNCC | | |
| | | NCCCNCCTNGCNGCTNGTNTACCNCANNNTTGNGNGGGNNACNGNCN | | |
| | | CNCNNCGNCNNA | | |
| prey93155 | 878 | TGCCTTATATTTTATGGCTATTTTTAATCACAGACGTTGTCAAACAT | 879 | CLIFYGYF*SQTLSNIQRRNNAXT |
| | | ACAAAGGAGAAACAACGCANAAACAAATCCCTTGAACCCGTCACCCA | | NPLNPSPNFSKSSTLSSSCPFTIV |
| | | ATTTCAGCAAATCCTCAACACTTTCCTCCTCTTGTCCATTTACGATT | | LNKHFRLDRPGARQGDPSA*FVFP |
| | | GTACTCAACAAGCACTTTCGACTGGACCGTCCAGGAGCGAGACAAGG | | PRALGVGLSPVGLALTSPLQVMLH |
| | | TGATCCTTCTGCTTGATTTGTTTTCCCACCGAGAGCTTTGGGGGTGG | | *SHDKSQYLSCFHFCTPEQVGL*E |
| | | GTCTGTCGCCCGTGGGGCTCGCCCTCACCTCGCCTCTTCAAGTGATG | | TETVLALPVGPSLAPAEHNWA |
| | | CTTCATTGAAGTCATGATAAATCTCAGTATCTATCATGTTTCCATTT | | |
| | | TTGTACACCAGAACAAGTGGGGCTTTGAGAGACAGAAACCGTCCTGG | | |
| | | CTCTTCCTGTGGGTCCCTCTCTCCCGCCTGCAGAACACAACTGGGCG | | |
| prey93159 | 880 | ACGGTTTATATCGATAATCTTCAATATAAAATACTGGCCATTTTATA | 881 | TVYIDNLQYKILAIL*NIKYI*IL |
| | | AAATATTAAATATATTTAAATTCTTTGCATTTCATATTATTTGGGTC | | CISYYLGPFICLNYIFKFRLPLEH |
| | | CCTTTATATGCCTAAATTACATCTTTAAGTTTCGGTTGCCATTGGAA | | CGNNSDYITFPIRFLADFILFCYF |
| | | CATTGTGGAAACAATTCAGATTATATCACGTTTCCCATCCGCTTTTT | | FXLVCYTGQSPRNVSF*GAFPDSF |
| | | AGCTGATTTCATTCTTTTCTGTTATTTTTTTGNTCTAGTATGTTATA | | SSNVVNGSLCS*WHLVXXNSFVSS |
| | | CAGGGCAAAGCCCTAGAAACGTTTCCTTTTGAGGAGCTTTCCCAGAT | | L |
| | | TCCTTTTCCTCAAATGTTGTAAACGGAAGTTTATGTAGTTAATGGCA | | |
| | | TCTGGTGNAANANAACAGCTTTGTTTCCTCTTTAAN | | |
| prey93160 | 882 | GGGCGACAAAGGGACCCGAGTGTTCAAGAAGGCCAGTCCAAATGGAA | 883 | GDKGTRVFKKASPNGKLTVYLGKR |
| | | AGCTCACCGTCTACCTGGGAAAGCGGGACTTTGTGGACCACATCGAC | | DFVDHIDLVDPVDGVVLVDPEYLK |
| | | CTCGTGGACCCTGTGGATGGTGTGGTCCTGGTGGATCCTGAGTATCT | | ERRVYVTLTCAFRYGREDLDVLGL |
| | | CAAAGAGCGGAGAGTCTATGTGACGCTGACCTGCGCCTTCCGCTATG | | TFRKDLFVANVQSFPPAPEDKKPL |
| | | GCCGGGAGGACCTGGATGTCCTGGGCCTGACCTTTCGCAAGGACCTG | | TRLQERLIKKLGEHAYPFTFEIPP |
| | | TTTGTGGCCAACGTACAGTCGTTCCCACCGGCCCCCGAGGACAAGAA | | NLPCSVTLQPGPEDTGKACGVDYE |
| | | GCCCCTCACGCGGCTGCAGGAACGCCTCATCAAGAAGCTGGGCGAGC | | VKAFCAENLEEKIHKRNSVRLVTR |
| | | ACGCTTACCCTTTCACCTTTGAGATCCCTCCAAACCTTCCATGTTCT | | KVQYAPERPGPQPT |
| | | GTGACACTGCAGCCGGGGCCCGAAGACACGGGGAAGGCTTGCGGTGT | | |
| | | GGACTATGAAGTCAAAGCCTTCTGCGCGGAGAATTTGGACGAGAAGA | | |
| | | TCCACAAGCGGAATTCTGTGCGTCTGGTCATCCGGAAGGTTCAGTAT | | |
| | | GCCCCAGAGAGGCCTGGCCCCCAGCCCACAG | | |
| prey93161 | 884 | TCTGCTTTGGGGAACACTTCTGGCACCACCAGTGGTACTTCGTATAG | 885 | SALGNTSGTTSGTSYRSYGII*GL |
| | | GTCTTATGGCATTATTTAAGGTTTAGGTTATTGCACTAAACAGGATG | | GYCTKQDAKYSRT*RDHFLLQYTI |
| | | CAAAATACTCAAGGACATAGAGAGATCACTTTTTACTGCAATACACA | | YWMNCSL*HD*GHMAF*MDTHNT* |
| | | ATTTACTGGATGAACTGTTCACTGTGACATGATTAGGGTCACATGGC | | IDHCNSNKRWLQNYYSSIVGNAVN |
| | | ATTTTAAATGGATACTCACAATACTTGAGATCATTGCAATAGCAACA | | FIQLRFILYCLHLFLFI*TVNGAM |
| | | AGAGGTGGTTACAAAATTATTACAGTAGTATAGTAGGTAATGCAGTT | | YVIEC |
| | | AATTTTATCCAATTACGATTTATACTGTATTGTTTACATTTGTTTCT | | |
| | | ATTTATCTAGACTGTGAATGGTGCCATGTATGTGATCGAGTGTGT | | |
| prey93163 | 886 | TTACTTTTTGTAAAATGCAAAATGGTATTTTACATTTTTTGACTATT | 887 | LLFVKCKMVFYIF*LFWHFIK*AY |
| | | TTGGCATTTTATAAATAAGCCTACATTTTTGGAGACATTTTTAAAGA | | IFGDIFKEANLF*NKINTSKTRDI |
| | | AGCCAATCTTTTCTAAAATAAAATTAATACATCAAAAACTAGAGACA | | NVVYIVY*ILQ*SVKFVYINVRLP |
| | | TAAATGTCGTATACATCGTTTACTGAATCCTACAGTAATCTGTAAAG | | KT**SFYFTLYLIYKSPKYIQTDK |
| | | TTTGTGTACATCAATGTTAGATTACCTAAAACATAATAAAGTTTCTA | | NCGCHFKKQCLSGHGGSCL*AQHF |
| | | TTTTACGTTATATTTAATATACAAAAGTCCAAAATATATCCAGACAG | | GRP |
| | | ACAAAAATTGTGGCTGCCACTTTAAAAAACAATGTTTGTCTGGCCAT | | |
| | | GGTGGCTCATGCCTGTAAGCCCAACACTTTGGAAGGCCA | | |
| prey93169 | 888 | CGTTGGGTACTACTTACGGTTTTATTTGAAGGAAGGACCCCAGAGTT | 889 | RWVLLTVLFEAGRTPELNSFEtTh |
| | | AAACTCATTTGAAACCTCTGATAAATTCAGTCCCCTCCCTCTTTTTA | | **IQSPPSFYR*GPERRYDLLKVT |
| | | CAGGTGAGGCCCGGAGAGAAGATATGACTTGCTTAAGGTCACACACT | | HLVSLSIFLNRGRVLFPLYQAAST |
| | | TAGTTTCTCTGTCAAACTTTCTAAATCGAGGACGAGTTCTCTTCCCG | | NREVQK*PNPGRRAVVCVSSNYFM |
| | | CTGTATCAGGCTGCCTCAACAAACACGCAAGTACAAAAATAGCCAAA | | GQPSLFS*FPLCPSCRISALCPIS |
| | | TCCAGGAAGGAGGGCAGTGGTCTGTGTGTCCTCCAATTATTTCATGG | | FGLAERTGCTSSLPSASPRWSNMQ |
| | | GACAGCCAAGCCTATTTTCTTGATTTCCCTTGTGCCCCTCCTGTCGC | | RERRAGARVKAGKETPRPPPVFPH |
| | | ATATCTGCTTTGGGACCCATCTCTTTTCCGCTTGCAGAGACAACGGG | | LYFWNFRLPPPPGPFLVAHQGEEE |
| | | CTGCACCACCTCTCTGCCCTCTGCATCACCCAGATGGTCAAACATGC | | GKLS*ISPIYTR*QSFLCQKKHRK |
| | | AAAGGGAAAGGAGAGCTCGGGCCAGAGTCAAGGCAGGCAAAGAGACT | | GA |
| | | CCTCGACCCCCTCCAGTCTTTCCACATTTATATTTTTCCAATTTCAG | | |
| | | GCTCCCTCCTCCCCCGGGACCTTTCCTAGTAGCCCACCAGGGAGAAG | | |
| | | AAGAGGGAAAACTGTCATAAATTTCTCCTATTTATACAAGATGACAG | | |
| | | AGTTTCTTGTGCCAGAAAAAACACAGAAAGCGACCTG | | |
| prey93176 | 890 | TATTTTTTGTAGAGACGAGGTTTTCACTATCTTGCCCAAGCTGAAAA | 891 | YFL*RRGFHYVAQAENSSFDDYIL |
| | | TTCTACTTTTGATGATTATATTTTACATTATTTAGAACAAGTTTCAT | | HYLEQVSCF*KSKAKLRESPY*AI |
| | | GTTTTTAAAAAAGCAAAGCAAAACTCAGAGAATCTCCTTATTGACCT | | LNRLLSXXXXXXXXXXXXXXXXXX |
| | | ATTTTGAATAGATTATTAAGTTTNNNNNNNNNNNNNNNNNNNNNNNN | | XXXXXXXXXXXXXXXXXXIHVNHH |
| | | NNNNNNNNNNNNNNNNNNNNNNNNNNNNNNNNNNNNNNNNNNNNNNN | | VCSXIDF*MXENH |
| | | NNNNNNNNNNNNNNNNNNNNNNNNNNNNNNNNNNNATACATGTAAAC | | |
| | | CACCATGTGTGCTCTNAATAGATTTTTAAATGANTGAGAATCACCT | | |
| prey56846 | 892 | GGAGCTCAAGAGCCGTCTGGACACCCTGGCCCAGGAGGTGGCCCTGC | 893 | ELKSRLDTLAQEVALLKEQQALQT |
| | | TGAAGGAGCAGCAGGCCCTGCAGACGGTCTGCCTGAAGGGGACCAAG | | VCLKGTKVHMKCFLAFTQTKTFHE |
| | | GTGCACATGAAATGCTTTCTGGCCTTCACCCACACGAAGACCTTCCA | | ASEDCISRGGTLSTPQTGSENDAL |
| | | CGAGGCCAGCGAGGACTGCATCTCGCGCGGGGGCACCCTGAGCACCC | | YEYLRQSVGNEAEIWLGLNDMAAE |
| | | CTCAGACTGGCTCGGAGAACGACGCCCTGTATGAGTACCTGCGCCAG | | GTWVDMTGARIAYKNWETEITAQP |
| | | AGCGTGGGCAACGAGGCCGAGATCTGGCTGGGCCTCAACGACATGGC | | DGGKTENCAVLSCAANGKWFDKRC |
| | | GGCCGAGCGCACCTGGGTGGACATGACCGGCGCCCGCATCGCCTACA | | RDQLPYICQFGIV* |
| | | AGAACTGGGAGACTGAGATCACCGCGCAACCCGATGGCGGCAAGACC | | |
| | | GAGAACTGCGCGGTCCTCTCAGGCGCGGCCAACGGCAAGTGGTTCGA | | |
| | | CAGCGCTGCCGCGATCAGCTGCCCTACATCTGCCAGTTCGGGATCGT | | |
| | | GTAG | | |
| prey93186 | 894 | TTATATTTTATAAGCTCAACGATAGTTTTATAACTTATTTCACATAG | 895 | IFYKLNDSFITYFT*LHFQLRKKP |
| | | TTGCATTTTCAATTAAGAAAAAAACCAGTCTGGGTCCAATGATTCAC | | VWVQ*ETPLILALWEAKGGGSLEA |
| | | ACCATTAATCCTAGCACTTTGGGAAGCCAAAGGAGGAGGATCACTTG | | RNMRHDETLSLQTFF*KLAGWGGA |
| | | AGGCCAGGAATATGAGACATGATGAGACACTCTCTCTACAGACATTT | | CLATREIEVGGWA |
| | | TTTTAAAAGTTAGCCGGGTGGGGTCGTGCATGCCTAGCTACTCGGGA | | |
| | | GATTGAAGTGGGAGGATGGGCT | | |
| prey84370 | 896 | AATTGAAGGACTTGAGGAGCCGAATGGCTCCTCCCTGAAGAACATAG | 897 | IEGLEEPNGSSLKNIEKYLRSQSD |
| | | AGAAGTATCTCAGAAGTCAAAGTGATCTCACAAGCACCACCAACAAC | | LTSTTNNPAFQQRLRLGAKRAVNN |
| | | CCAGCCTTTCAGCAGCGGCTGCGACTGGGGGCCAAACGCGCTGTGAA | | GRLLKDGPQYRVNYGSLDGKGAPQ |
| | | TAATGGGAGGTTACTGAAAGACGGACCGCAGTACAGGGTCAATTATG | | YPSAFPSSLPPVSLLPHEKDQPRA |
| | | GGAGCTTAGATGGCAAAGGGGCACCTCAGTATCCCAGTGCATTCCCA | | DPIPICSFCLGTKESNREKKPEEL |
| | | TCCTCGCTCCCACCTGTCAGCCTTCTACCCCATGAGAAAGACCAGCC | | LSCADCGSSGHPSCLKFCPELTTN |
| | | CCGTGCTGATCCCATTCCAATATGTAGCTTCTGTTTGGGGACTAAAG | | VKALRWQCIECKTCSACRVQGRNA |
| | | AATCAAATCGTGAAAAGAAACCAGAAGAACTCCTCTCTTGTGCAGAT | | DNMLFCDSCDRGFHMECCDPPLSR |
| | | TGTGGCAGTAGTGGACACCCATCCTGTTTGAAATTTTGTCCTGAATT | | MPKGMWICQVCRPKKKGRKLLHEK |
| | | AACAACAAATGTAAAGGCCTTAAGGTGGCAGTGCATCGAATGCAAGA | | AAQIKRRYAKPIGRPKNKLKQRLL |
| | | CATGCAGTGCCTGTAGAGTCCAAGGCAGAAATGCTGATAATATGCTT | | SVTSDEGSMNAFTGRGSPGRGQKT |
| | | TTTTGTGATTCCTGTGATAGAGGATTTCATATGGAATGCTGTGACCC | | KVCTTPSSGHAASGKDSSSRLAVT |
| | | ACCACTTTCCAGAATGCCAAAAGGGATGTGGATTTGCCAAGTCTGCA | | DPTRPGATTKITTTSTYISASTLK |
| | | GACCAAAGAAAAAGGGAAGAAAACTACTTCATGAGAAAGCTGCACAA | | VNKKTKGLIDGLTKFFTPSPDGRR |
| | | ATAAAACGACGATATGCAAAACCCATTGGACGACCGAAAAATAAATT | | SRGEIIDFSKHYRPRKKVSQKQSC |
| | | AAAGCAACGATTGTTGTCTGTAACCAGTGATGAAGGATCCATGAATG | | TSHVLATGTTQKLKPPPSSLPPPT |
| | | CATTCACAGGAAGGGGGTCACCTGGTAGGGGTCAAAAGACTAAAGTC | | PISGQSPSSQKSSTATSSPSPQSS |
| | | TGTACCACACCTTCATCTGGTCATGCTGCATCTGGGAAGGACTCAAG | | SSQCSVPSLSSLTTNSQLKALFDG |
| | | CAGCAGATTGGCTGTTACAGACCCCACTCGGCCTGGTCCCACCACCA | | LSHTIPLRDSLAKRDTPSYAPPKR |
| | | AAATCACCACCACCTCCACCTACATTTCTCCCTCTACACTTAAAGTT | | MRRKTELSSTAKSKAHFFGKRDIR |
| | | AACAAGAAAACCAAAGGGCTCATTGATGGCCTTACTAAGTTTTTTAC | | SRFISHSSSSSWGMARGSIFKAIA |
| | | ACCATCACCTGATGGTCGCAGATCACGAGGTGAAATTATAGACTTTT | | HFKRTTFLKKHRMLGRLKYKVTPQ |
| | | CAAAGCACTATCGTCCAAGGAAAAAGGTCTCTCAGAAACAGTCATGC | | MGTPSPGKGSLTDGRIKPDQDDDT |
| | | ACTTCTCATGTGTTGGCTACAGGTACCACACAAAAGCTAAAACCTCC | | EIKINIKQESADVNVIGNKDVVTE |
| | | ACCTTCTTCACTTCCACCCCCAACCCCCATCTCCGGTCAGAGCCCCA | | EDLDVFKQAQELSWEKIECESGVE |
| | | GTTCACAAAAGTCCAGCACGGCCACTTCTTCTCCCTCTCCCCAGAGT | | DCGRYPSVIEFGKYEIQTWYSSPY |
| | | TCTTCCAGCCAGTGCAGTGTGCCCTCCCTGAGCAGCCTTACCACTAA | | PQEYARLPKLYLCEFCLKYMKSKN |
| | | CAGCCAGCTGAAGGCACTCTTTGATGGGCTTTCTCATACTATACCAC | | ILLRHSKKCGWFHPPANEIYRRKD |
| | | TCAGGGACAGTCTCGCAAAAAGGGACACCCCGAGTTATGCACCACCC | | LSVFEVDGNMSKIYCQNLCLLAKL |
| | | AAACGTATGCGTCGTAAAACTGAATTATCTTCCACGGCAAAATCTAA | | FLDHKTLYYDVEPFLFYVLTKNDE |
| | | AGCCCACTTCTTTGGCAAAAGAGATATTAGAAGTCGGTTTATTTCTC | | KGCHLVGYLSKEKLCQQKYNVSCT |
| | | ACTCCTCCTCCTCTAGCTGGGGGATGGCTAGAGGAAGTATTTTTAAA | | MIMPQHQRQGFGRFLIDFSYLLSR |
| | | GCAATTGCTCACTTCAAGCGAACAACTTTCCTTAAAAAGCACAGGAT | | REGQAGSPEKPLSDLGRLSYLAYW |
| | | GCTAGGCAGATTAAAATATAAAGTGACCCCTCAGATGGGGACCCCCT | | KSVILEYLYHHHERHISIKAISRA |
| | | CACCAGGGAAGGGGAGCTTGACAGACGGAAGGATTAAACCTGATCAG | | TGMCPHDIATTLQHLHMIDKRDGR |
| | | GATGATGATACTGAAATAAAAATAAACATCAAACAAGAAAGTGCAGA | | FVIIRREKLILSHMEKLKTCSRAN |
| | | TGTAAATGTGATTGGAAACAAGGATGTCGTTACTGAAGAGGATTTGG | | ELDPDSLRWTPILISNAAVSEEER |
| | | ATGTTTTTAAGCAGGCCCAGGAACTTTCTTGGGAGAAAATAGAGTGT | | EAEKEAERLMEQASCWEKEEQEIL |
| | | GAGAGTGGGGTGGAAGACTGTGGCCGGTACCCTTCTGTGATTGAATT | | STRANSRQSPAKVQSKNKYLHSPE |
| | | TGGTAAATATGAAATCCAAACCTGGTACTCCTCGCCTTACCCACAGG | | SRPVTGERGQLLELSKESSEEEEE |
| | | AATATGCAAGATTACCAAAGCTTTACCTGTGTGAATTCTGTCTTAAA | | EEDEEEEEEEEEEEEDEEEEEEEE |
| | | TATATGAAAAGTAAAAATATTTTGCTAAGACACTCCAAGAAGTGTGG | | EEEEEENIQSSPPRLTKPQSVAIK |
| | | ATGGTTTCATCCTCCAGCAAATGAAATTTACCGAAGGAAAGACCTTT | | RKRPFVLKKKRGRKRRRINSSVTT |
| | | CAGTATTTGAGGTTGATGGGAATATGAGCAAAATTTATTGCCAAAAC | | ETISETTEVLNEPFDNSDEERPMP |
| | | CTTTGCTTGTTAGCCAAGCTCTTCCTGGACCACAAAACGTTGTATTA | | QLEPTCEIEVEEDGRKPVLRKAFQ |
| | | TGATGTCGAGCCATTCCTTTTTTATGTCCTTACAAAAAATGATGAAA | | HQPGKKRQTEEEEGKDNHCFKNAD |
| | | AGGGCTGTCATCTGGTTGGATACCTCTCTAAGGAAAAGCTTTGCCAG | | PCRNNMNDDSSNLKEGSKDNPEPL |
| | | CAGAAGTATAATGTCTCCTGCATAATGATCATGCCCCAGCACCAAAG | | KCKQVWPKGTKRGLSKWRQNKERK |
| | | GCAAGGATTTGGACGGTTTCTCATTGATTTCAGCTATTTGCTTTCTA | | TGFKLNLYTPPETPMEPDEQVTVE |
| | | GAAGAGAAGGCCAAGCAGGGTCTCCTGAAAAGCCTCTCTCCGATCTG | | EQKETSEGKTSPSPIRTEEEVKET |
| | | GGCCGTCTCTCCTACCTGGCATATTGGAAGAGCGTCATCTTGGAGTA | | GEALLPQEENRREETCAPVSPNTS |
| | | TCTCTACCACCACCATGAGAGGCACATCAGCATCAAGGCAATTAGCA | | PGEKPEDDLIKPEEEEEEEEEEEE |
| | | GAGCGACGGGCATGTGCCCACATGACATTGCCACCACTCTGCAGCAC | | EEEEEEGEEEEGGGNVEKDPDGAK |
| | | CTCCACATGATCGACAAGAGAGATGGCAGATTTGTCATCATTAGACG | | SQEKEEPEISTEKEDSARLDDHEE |
| | | GGAAAAGTTGATATTGAGCCACATGGAAAAGCTGAAAACCTGTTCCA | | EEEEDEEPSHNEDHDADDEDDSHM |
| | | GAGCCAATGAACTTGATCCAGACAGTCTGAGGTGGACCCCAATTTTA | | ESAEVEKEELPRESFKEVLENQET |
| | | ATTTCTAATGCTGCAGTGTCTGAAGAAGAGCGAGAAGCTGAGAAAGA | | FLDLNVQPGHSNPEVLMDCGVDLT |
| | | GGCTGAGCGGCTAATGGAACAAGCTAGCTGCTGGGAGAAGGAGGAAC | | ASCNSEPKELAGDPEAVPESDEEP |
| | | AAGAAATCCTGTCAACTAGAGCTAACAGTAGGCAATCACCTGCAAAA | | PPGEQAQKQDQKNSKEVDTEFKEC |
| | | GTACAATCGAAAAATAAATATTTGCATTCCCCGGAGAGCCGGCCAGT | | NPATMEIDSETVQAEACRSLQNYT |
| | | CACAGGGGAGCGAGGGCAGCTGCTGGAGCTGTCTAAAGAGAGCAGTG | | RADQSPQIATTLDDCQQSDHSSPV |
| | | AAGAAGAAGAGGAGGAGGAGGACGAGGAGGAGGAAGAAGAGGAGGAA | | SSVHSHPGQSVRSVNSPSVPALEN |
| | | GAAGAGGAAGAGGATGAAGAGGAGGAAGAAGAGGAAGAAGAAGAAGA | | SYAQISPDQSAISVPSLQNMETSP |
| | | AGAAGAAGAAAATATTCAAAGCTCTCCCCCAAGATTGACGAAACCAC | | MMDVPSVSDHSQQVVDSGFSDLGS |
| | | AGTCAGTTGCCATAAAGAGAAAGAGGCCTTTTGTACTAAAGAAGAAA | | IESTTENYENPSSYDSTMGGSICG |
| | | AGGGGTCGTAAACGCAGGAGGATCAACAGCAGTGTAACAACAGAGAC | | NGSSQNSCSYSNLTSSSLTQSSCA |
| | | CATTTCAGAGACGACAGAAGTACTGAATGAGCCCTTTGACAACTCAG | | VTQQMSNISGSCSMLQQTSISSPP |
| | | ATGAAGAGAGGCCAATGCCACAGCTGGAGCCTACCTGTGAGATTGAA | | TCSVKSPQGCVVERPPSSSQQLAQ |
| | | GTGGAGGAAGATGGCAGCAAGCCAGTCCTGAGAAAAGCATTCCAGCA | | CSMAANFTPPMQTAAEIPETSNAN |
| | | TCAGCCTGGGAAGAAAAGACAAACAGAGGAAGAGGAAGGAAAAGACA | | IGLYERMGQSDFGAGHYPQPSATF |
| | | ATCATTGCTTCAAGAATGCTGACCCTTGTAGAAACAATATGAATGAT | | SLAKLQQLTNTLIDHSLPYSHSAA |
| | | GATTCAAGTAACTTGAAAGAACGCAGTAAAGACAATCCCGAACCTCT | | VTSYANSASLSTPLSNTGLVQLSQ |
| | | AAAGTGCAAACAAGTCTGGCCAAAAGGAACAAAGCGCGGTCTATCTA | | SPHSVPGGPQAQATMTPPPNLTPP |
| | | AGTGGAGGCAAAACAAAGAGAGGAAGACCGGATTTAAACTGAATTTG | | PMNLPPPLLQRNMAASNIGISHSQ |
| | | TACACCCCGCCAGAAACACCCATGGAGCCTGACGAGCAGGTAACAGT | | RLQTQIASKGHISMRTKSASLSPA |
| | | GGAAGAACAGAAGGAGACTTCAGAAGGAAAAACCAGCCCCAGTCCCA | | AATRQSQIYGRSQTVAMQGPARTL |
| | | TCAGGATTGAGGAGGAGGTCAAGGAAACTGGGGAAGCCCTGTTGCCT | | TMQRGMNMSVNLMPAPAYNVNSVN |
| | | CAACAGGAAAACAGAAGGGAAGAAACATGTGCCCCTCTAAGTCCAAA | | MNMNTLMNGYSMSQPMMNSGYHSN |
| | | CACATCACCAGGTGAAAAACCAGAAGATGATCTCATCAAACCTGAGG | | HGYMNQTPQYPMQMQMGMMGTQPY |
| | | AAGAGGAAGAGGAGGAGGAGGAGGAAGAGGAAGAAGAGGAAGAAGAG | | AQQPMQTPPHGNMMYTAPGHHGYM |
| | | GAACGGGAAGAAGAAGAAGGAGGAGGAAATGTAGAAAAAGATCCAGA | | NTGMSKQSLNGSYMR |
| | | TGGTGCTAAAAGCCAAGAAAAAGAGGAACCAGAAATCTCCACGGAAA | | |
| | | AAGAAGACTCTGCACGTTTGCATGATCACGAAGAGGAGGAGGAAGAG | | |
| | | GATGAAGAGCCATCCCACAACGAGGACCATGATGCCGATGACGAGGA | | |
| | | TGACAGCCACATGGAGTCTGCCGAAGTGGAGAAGGAAGAGCTGCCCA | | |
| | | GAGAAAGCTTCAAAGAAGTACTGGAAAACCAVQSLTQESSEQDDTFQ | | |
| | | DCAETQGGAGACTTTTTTAGACCTTAATGTGCAGCCTGGTCACTCGA | | |
| | | ACCCAGAGGTCTTAATGGACTGTGGCGTCGACCTGACAGCTTCTTGT | | |
| | | AACAGTGAGCCCAAGGAGCTTGCTGGGGACCCTGAAGCTGTACCCGA | | |
| | | ATCTGACGAGGAGCCACCCCCAGGAGAACAGGCACAGAAGCAGGACC | | |
| | | AAAAGAACAGCAAGGAAGTCGATACAGAGTTCAAAGAGGGAAACCCA | | |
| | | GCAACCATGGAAATCGACTCTGAGACTGTCCAGGCCGTTCAGTCTTT | | |
| | | GACCCACGAGAGCAGCGAACAGGACGACACCTTTCAGGATTGTGCCG | | |
| | | AGACTCAAGAGGCCTGTAGAAGCCTACAGAACTACACCCGTGCAGAC | | |
| | | CAAAGTCCACAGATTGCCACCACGCTCGACGATTGCCAACAGTCGGA | | |
| | | CCACAGTAGCCCAGTTTCATCCGTCCACTCCCATCCTGGCCAGTCCG | | |
| | | TACGTTCTGTCAACAGCCCAGTGTCCCTGCTCTGGAAAACAGCTACG | | |
| | | CCCAAATCAGCCCAGATCAAAGTGCCATCTCAGTGCCATCTCTGCAG | | |
| | | AACATGGAAACCAGTCCCATGATGGATGTCCCATCAGTTTCAGATCA | | |
| | | TTCACAGCAAGTCGTAGACAGTGGATTTAGTGACCTGGGCAGTATCG | | |
| | | AGAGCACAACTGAGAACTACGAAAACCCAAGCAGCTACGATTCTACT | | |
| | | ATGGGAGGCAGCATCTGTGGAAACGGCTCTTCACAGAACAGCTGCTC | | |
| | | CTATAGCAACCTCACCTCCAGCAGTCTGACACAGAGCAGCTGTGCTG | | |
| | | TCACCCAGCAGATGTCCAACATCAGCGGGAGCTGCAGCATGCTGCAG | | |
| | | CAAACCACCATCAGCTCCCCTCCGACCTGCAGCGTCAAGTCTCCTCA | | |
| | | AGGCTGTGTGGTGGAGAGGCCTCCGAGCAGCAGCCAGCAGCTGGCTC | | |
| | | AGTGCAGCATGGCTGCTAACTTCACCCCACCCATGCAGCTGGCTGAA | | |
| | | ATCCCCGAGACGAGCAACGCCAACATTGGCTTATACGAGCGAATGGG | | |
| | | TCAGAGTGATTTTGGGGCTGGGCATTACCCGCAGCCGTCAGCCACCT | | |
| | | TCAGCCTTGCCAAACTGCAGCAGTTAACTAATACACTTATTCATCAT | | |
| | | TCATTGCCTTACAGCCATTCCGCTGCTGTGACTTCCTATGCAAACAG | | |
| | | TGCCTCTTTGTCCACACCATTAAGTAACACAGGGCTTGTTCAACTTT | | |
| | | CTCAGTCTCCACACTCCGTCCCTGGGGGACCCCAAGCACAAGCTACC | | |
| | | ATGACCCCACCCCCCAACCTGACTCCTCCTCCAATGAATCTGCCGCC | | |
| | | GCCTCTTTTGCAACGGAACATGGCTGCATCAAATATTGGCATCTCTC | | |
| | | ACAGCCAAAGACTGCAAACCCAGATTGCCAGCAAGGGCCACATCTCC | | |
| | | ATGAGAACCAAGTCAGCGTCTCTGTCACCAGCCCCTGCCACCCATCA | | |
| | | GTCACAAATCTATGGGCGCTCCCAGACTGTAGCCATGCAGGGTCCTG | | |
| | | CACGGACTTTAACGATGCAAAGAGGCATGAACATGAGTGTGAACCTG | | |
| | | ATGCCACCGCCAGCCTACAATGTCAACTCTGTGAACATGAACATGAC | | |
| | | ACTCTCAACGCCATGAATGGGTACAGCATGTCCCAGCCATGATGACA | | |
| | | GTGCCTACCACAGCAATCATGGCTATATGAATCAAACGCCCCAATAC | | |
| | | CCTATGCAGATGCAGATGGGCATGATGGGCACCCAGCCATATGCCCA | | |
| | | GCAGCCAATGCAGACCCCACCCCACGGTACATGATGTACACGGCCCC | | |
| | | CGGACATCACGGCTACATGAACACAGGCATGTCCAAACAGTCTCTCA | | |
| | | ATGGCTCCTACATGAGAAGGTAG | | |
| prey93188 | 898 | GTTAGTTCACCCCTGCAGAACAAACATTCAAAGGAGGCAAGTTTCAC | 899 | VSSPLQNKHSKEASFTKIINPGHR |
| | | TAAGATAATTAACCCTGGTCATCGCAGGGTGACTCCAAAGAGAATTT | | RVTPKRIWAGAFRCTLTVPYQNCA |
| | | GGGCTGGTGCATTCCGATGCACCTTGACCGTCCCATACCAGAATTGT | | RCTWNFPRQM*ILRGVFCPLVRKS |
| | | GCCAGGTGCACCTGGAATTTTCCAAGACAGATGTGATCTTAAGGGGA | | TLKCAKLRNQSLRKTQTPFWPNLK |
| | | GTATTTTGTCCTCTTGTGAGGAAAAGTACTTTAAAATGTGCAAAACT | | G*SCC*L*LPLAIPHEHQPRVLIX |
| | | GAGGATCAGAGCTTAAGGAAGACTCAGACCCCATTCTGGCCAAACTT | | VG*SCLGTLVFTAGNLIT*AXXP*LK |
| | | GAAAGGCTGATCTTGTTGCTAACTTTGATTGCCACTTGCTATCCCAC | | |
| | | ATGAGCATCAACCCAGAGTTCTCATANGTGTTCGGTGATCTTGTTTG | | |
| | | GGAACCTTGGTTTTCACAGCAGGGAACCTGATTACTTAAGCAANGNG | | |
| | | CCCCTAATTGAAAN | | |
| prey93189 | 900 | ACGGGGCAAGCTACGTTACATTATGGCAGAAAAAAAGTGCAACTGAC | 901 | TGQATLHYGRKKVQLTYYKEIF*V |
| | | ATACTACAAAGAGATTTTTTAAGTTTAAAAAAAGTTTGGATCTTTTG | | *KKFGSFGFLFFFLGFXGLKNP*X |
| | | GATTTCTTTTTTTTTTTTTGGGGTTTNNGGGCCTAAAAAACCCNTAA | | *LPPPKREVFFFFLGPPQNTRXGV |
| | | TTNTAATTACCCCCCCCAAAAAGGGAGGTTTTTTTTTTTTTTTTGGG | | FXFPPLSGFXGXPSPKEXPXFSPQ |
| | | CCCCCCCCAAAACACCCGGNTAGGGGTTTTTNCCTTTCCCCCCCTTT | | GGPLXKPPKKNRLQXGXGGTLPLX |
| | | CCGGNTTTNGNGGGGNCCCATCCCCAAAGGAGNCCCCCTTNTTTTCC | | FWKKKAPPXNHFFXKF |
| | | CCCCAAGGGGCNCCTTTGNNAAACCNCCNAAAAAAAACCGGCTCCAA | | |
| | | NGGGGGNCAGGGGGAACCCTNCCCCTTTTNTTTTGGAAAAAAAAGGC | | |
| | | CCCCCCCNAAAACCACTTTTTTNGNAAATTT | | |
| prey93193 | 902 | CCGGGGCAAGCTACGTTACATTATGGCANGAAAAAAAGTGCAACTGA | 903 | PGQATLHYGXKKSATDILQRDFLS |
| | | CATACTACAAAGAGATTTTTTAAGTTAAAAAAAGTTTGGATCTTTTG | | *KKFGSFGFLFFFWXXXGXKXPXF |
| | | GATTTCTTTTTTTTTTTTGGGNNTTNNGGGGCNAAAAAANCCCNNAA | | XXPPXKXXXFFFFXGPPQXXXXXX |
| | | TTTNAANAACCCCCNNNAAAAANGNNNNTTTTTTTTTTTTTTTNNGG | | XXFXPXXXFXGXXXPXXPPXFXPX |
| | | CCCNCCCCAAAANANCCNNGANNGGGNNNTTNNCTTTNNNCCCCNTN | | XGPLXXXXXKXXXKRGXGXXXPXF |
| | | NNNGNTTTNNNGGNNNNCNNNCCCCAANNAANCCCCCCTTNTTTNNC | | XXKX |
| | | CCCNANNGGGGGCCTTTGGNAAANNNNNNAAANAAAANCNNCNCCAA | | |
| | | AAGGGGGCNNGGGGNIAINCCNNNCCCNNTTTTNTTNANAAAANNAN | | |
| prey93194 | 904 | TTNANTATAACATTATACAGCTCTTTCTGAGTCTTTCTGTACCTCAN | 905 | XXITLYSSF*VFLYLXFIIWK*E* |
| | | TTTCATAATATGGAAATGAGAGTAGTAATACCTATGACGCAGGCTTA | | *YL*RRLTTRLIXVCNSVLISLKK |
| | | CNACGAGGCTTATATANGTTTGTAATTCAGTACTTATATCTCTAAAA | | VIX*FKAIKNLTTSXFHY**LFLE |
| | | AAAGTTATCANGTAATTTAAAGCAATAAAGAACCTAACAACCTCTNC | | FLFQSLIF*LIFHKRLFPSILVDT |
| | | TTTTCATTACTGATAACTTTTCTTAGAGTTTCTGTTTCAGTCGTTAA | | EIQR*MKHSPVPQETSLAXKIDX* |
| | | TTTTCTAATTAATTTTTCATAAACGACTATTTCCGAGTATTCTGGTG | | TIITXCDAL |
| | | GACACTGAGATACAAAGATGAATGAAACACAGTCCTGTTCCCCAAGA | | |
| | | GACTAGTCTAGCANTTAAGATTGACNTGTAAACCATAATAACAGNGT | | |
| | | GTGATGCTTTATT | | |
| prey93197 | 906 | TGGTCTGAATATAACTTTTTGCCTTTTTGCCTTTGGCAGGATCTGGG | 907 | WSEYNFLPFCLWQDLGIVAXSHLQ |
| | | CATAGTATAATNATCACACCTCCAGGAATAGCTATTTATTTAGTCAA | | E*LFI*SKNTVHLLCSRP*T*SC* |
| | | AAAATACAGTGCATCTATTATGTTCCAGGCCCTGAACGTAAAGTTGT | | HTLSRGDYI*SCIADHVFPCP*LE |
| | | TAACACACCTTGTCTCGTGGTGATTATATCTGATCATGTATTGCTGA | | HRNWQTGRMDYCFLFIFIF*NILL |
| | | TCATGTGTTCCCATGTCCTTAACTAGAACATAGAAATTGGCAGACAG | | GQS*FKLASFINTVAYWLGCVHLH |
| | | GTAGAATGGACTACTGCTTCTTATTTATTTTTATTTTTTAAAATATT | | VTSTIWKR |
| | | CTGCTTGGGCAAAGCTAATTCAAGTTAGCATCCTTTATAAATACAGT | | |
| | | AGCATATTGGTTAGGTGGTGTTCACTTACACGTAACTTCAACTATTT | | |
| | | GGAAGAGGA | | |
| prey93201 | 908 | CCTACAACCCCTACAACCATTTCACCTCTTTGAAATTTTATAAAGGC | 909 | PTTPTTISPL*NFIKAYFKLEK*F |
| | | ATATTTTAAACTTGAAAAGTAATTTGTCAACTACCTTACCTTTCTAA | | VNYLTFLTSHIILLTIKLFHRSKL |
| | | CTTCACATATAATTTTGTTAACAATAAAACTATTTCACAGATCAAAG | | *PTETKYRVQQHRTKCFSSSLCRS |
| | | TTGTAACCCACAGAAATCAAATACAGAGTGCAACAGCACAGGACAAA | | IKSTIQQATNPPNTHITFSHLVHK |
| | | ATGCTTCTCTTCCAGCTTGTGCAGATCCATCAAATCTACTATTCAAC | | DSLLLN*IFR*NQKTAFP*LITYL |
| | | AAGCTACAAATCCACCTAATACCCATATCACATTTTCCCATCTTGTT | | NFSESIFKGKQD*HRMTYFSLS*V |
| | | CATAAGGACAGCCTTTTGTTAAACTAAATATTCCGTTGAAATCAAAA | | PLGDLGPLQSLTSLL*EFPMS |
| | | GACAGCATTCCCCTAACTAATAACCTATTTAAACTTTTCAGAATCTA | | |
| | | TCTTTAAAGCAAAACAAGATTAGCACAGAATGACTTATTTTAGTCTC | | |
| | | TCCTAAGTCCCTCTGGGCGACTTAGGACCACTGCAATCCCTAACTTC | | |
| | | ACTCTTGTAAGAGTTTCCTATGTCCCC | | |
| prey93202 | 910 | GAGGGGGTAGACAAAATGTCATTCTATGGGTATCTTTGAGAGACCAG | 911 | EGVDKMSFYGYL*ETS*SYFSKQK |
| | | TTAAAGTTATTTCTCTAAGCAGAAACTGGGTCTTTTATTTCTGTACA | | LGLLFLYRQQFFTRPGGIK*VWAK |
| | | GACAACAGTTTTTCACAAGGCCTGGTGGTATAAAATAGGTCTGGGCC | | SGNRFLRHLVRILKYDERSGPCLT |
| | | AAGAGTGGCAACAGATTTCTGAGACACTTGGTGAGGATTCTTAAGTA | | AIASATGKGLWKRNIKSVYMKQII |
| | | TGATGAGAGAAGTGGGCCATGTTTAACTGCAATGTCTGCCACGGGCA | | GSTQEFENSXTHHFYGQF*DGHKE |
| | | AGGGACTATGGAAACGTAATATCAAATCAGTCTACATGAAACAGATC | | KNKNVSVVLMTWMLYSYYWHAD*F |
| | | ATAGGAACCACCCAAGAATTTGAAAATAGCANTACTCACCATTTTTA | | AC*YNIFS*GSIYSPGNSMV*RKR |
| | | TCGACAATTCTAAGATGGTCATAAAGAAAAAAATAAAAATGTGTCAG | | KGKPLLWNATNSHCIVEYVTELL* |
| | | TGGTGCTAATGACTTGGATGTTATATAGTTACTACTGGCATGCAGAT | | ASISLPMK*AGWTK*LLMSISSSW |
| | | TAATTTGCATGTTGATATAATATATTTTCATGAGGAAGTATATACTC | | TLCTFLSLKKPTFTFAEFTEKHMI |
| | | ACCAGGAAACAGCATGGTGTAACGGAAACGTAAAGGTAAGCCTTTAC | | HKYVNHSNQVRQA |
| | | TCTGGAATGCCACAAACTCTCACTGTATCGTTGAATATGTCATTGAA | | |
| | | CTTCTTTAGGCCTCAATTTCTCTACCCATGAAATGAGCTGGCTGGAC | | |
| | | TAAATGATTGCTAATGTCCATTTCCAGTTCTTGGACACTGTGTACCT | | |
| | | TTCTATCTTTAAAAAAACCTACTTTTACCTTCTAAGAATTTACAGAA | | |
| | | AAACACATGATCCACAAATATGTAAATCATAGTAACCAGGTAAGACA | | |
| | | AGCAC | | |
| prey93207 | 912 | GCGTGGGTTTGTTTTGGTTGGTTTTAGGCAATCCTAGCCACCCTTGG | 913 | AWVCFGWF*AILATLGFPIXLHCL |
| | | CTTTCCCATTANACTGCATTGCCTCTTTACGTTAAGCTTCCTTCATT | | FTLSFLH*IAXARIPK*DQLNFTV |
| | | AAATAGCCNCTGCCAGAATACCTAAGTGAGACCAACTGAATTTTACT | | E*LGSX*LFIRVVMVRSFQFKAES |
| | | GTTGAATAATTGGGCTCANTTTAGCTTTTCATCAGGGTTGTCATGGT | | SQLWEASQA*VQLTXGCTWRLMEK |
| | | ACGGTCATTTCAGTTCAAAGCTGAGTCAAGTCAGCTTTGGGAAGCCT | | TGP*FRSKWFSVDSLSIAILPLRW |
| | | CCCAGGCCTGAGTTCAGTTGACTCANGGCTGTACTTGGCGGCTTATG | | GGLPSYTPXD*XNFFTPTHFPFXN |
| | | GAAAAAACGGGGCCCTGATTCAGGTCTAAGTGGTTCAGTCTGGACAG | | RAXEG |
| | | TTTGTCACACCTCCCTCTTAGGTGGGGTGGGCTTCCCAGCTACACNC | | |
| | | CTTTNGATTGANGAAACTTTTTCACACCAACCCACTTTCCNTTTTTN | | |
| | | AACAGAGCGTNGGAAGGGG | | |
| prey93208 | 914 | GCATGGCCAAAAATGGAATAGGTAAATATTTCTGTGTTGACAGGGAA | 915 | AWPKME*VNISVLTGNNGR*GRMQ |
| | | CAATGGCAGATAGGGAAGGATGCAAACAACGTTGTACTATACTCCAA | | TTLYYTPICD*KINPH*VKKKIQE |
| | | TCTGTGATTAAAAAATTAACCCCCATTAAGTTAAAAAAAAAATACAA | | ESQYIDLIGQLKV*PFKQSIYSXI |
| | | GAATTCTCACAATACATAGATTTGATAGGACAGCTAAAGGTCTAACC | | PEMLFKTSKHSETHQKVI*HVLKD |
| | | TTTTAAGCAATCAATTTATTCANAAATTCCTGAGATGTTATTTAAAA | | LL*SIIFLSVLNSDFLLKQHQLCL |
| | | CTTCCAAACATTCTGAAACACATCAAAAAGTAATTTGACATGTTCTT | | FLLFLLLIFYIALALXSFNSXSVR |
| | | AAACATTTACTATAATCAATTATATTTTTAAGTGTTCTAAATAGCGA | | XEXCSLFMRLVWALNENINKVHQN |
| | | TTTTCTCTTAAAACAGCATCAATTATGCTTATTTCTGCTTTTTCTGT | | GNFSRSNLQSQIFFELITLRNF*V |
| | | TATTAATATTTTATATTGCCCTTGCCCTTNTGAGCTTCAATTCCTNA | | KT*KVFQYMQLKVLSXGQGAPRVA |
| | | TCTGTAAGGTNTGAAATNTGCAGCCTATTCATGCGCCTGGTGTGGGC | | SV*RPGTQ |
| | | NTTAAATGAGAACATAATAAGGTGCACCAAAATGGTAACTTCTCACG | | |
| | | TTCTAATCTTCAAAGCCAGATTTTCTTCGAACTCATAACGTTACGGA | | |
| | | ACTTCTAAGTGAAGACCTGAAAGGTTTTCCAATACATGCAACTTAAA | | |
| | | GTTTTAAGTNTGGGCCAGGGGGCCCCTCGAGTAGCTAGTGTCTAGAG | | |
| | | GCCCGGTACCCAA | | |
| prey93214 | 916 | GGAAGCATAATTTTGGGATTTCTTTAAATCTAATGGCAGCTGATCTC | 917 | GSIILGFL*I*WQLISEF*WDHLG |
| | | AGAATTCTGATGGGATCATCTTGGAGTTACGGAAATCCTATTAAGAG | | VTFILLRAKHSFYLFC*VLGVLFL |
| | | CAAAGCACTCTTTTTATCTCTTTTGCTAGGTCTTAGGTGTGTTATTT | | KNNVLFCVVILCLGLGTRSYLPNI |
| | | TTAAAAAATAATGTGCTTTTTTGTGTGGTGATCCTATGCTTGGGACT | | LVVS*KYILG*TFLTRTYHV*TLH |
| | | TGGCACTAGAAGTTATCTTCCAAATATACTGGTGGTTTCCTAGAAGT | | FGLNS*DM*GYLCHDKLFSYGRNG |
| | | ATATATTGGGTTAAACATTTCTAACTAGAACTTATCATGTATGAACA | | LIFRTNKFFGS**DLKDSSCCI*R |
| | | TTGCATTTTGGATTGAATAGCTAAGATATGTAAGGATATCTCTGCCA | | AFPICFKSGGRK*LCRYVL*LSI* |
| | | TGATAAATTATTTTCTTATGGAAGAAATGGTCTCATTTTCAGAACTA | | IFKVL*YRS*P*AR |
| | | ACAAATTTTTTGGGAGTTGATAGGATTTAAAGGACAGTTCATGCTGT | | |
| | | ATATAGAGAGCATTTCCTATTTGTTTCAAGTCAGGTGGGAGAAAGTA | | |
| | | ACTATGTAGGTATGTATTGTAGTTATCTATTTGAATTTTCAAGGTTC | | |
| | | TTTAGTACCGGTCTTAACCTTAAGCGCGAG | | |
| prey93217 | 918 | TTTCTATGTTATTTATAAAATTTTTTNACTGAAAATGTTTCTCAGAG | 919 | FLCYL*NFXTENVSQRCSGFYLTQ |
| | | ATGTTCTGGATTTTATCTGACTCAGTTTTTCTAGTTTTTATTCAATG | | FF*FLFNDHKEA*GKMPQAM*VRN |
| | | ATCATAAAGAGGCCTAAGGAAAAATGCCACAGGCAATGTGAGTTAGA | | SQVSQAPII*KHLXXXXXXXXXXX |
| | | AACTCTCAGGTTTCACAAAAGGCTCCCATCATTTAAAAACACTTGTA | | XXXXXXXXXXXXXXXXXXXXXXXX |
| | | NNNNNNNNNNNNNNNNNNNNNNNNNNNNNNNNNNNNNNNNNNNNNNN | | XXXXXXXXXXXXXXXXXXXCL*CH |
| | | NNNNNNNNNNNNNNNNNNNNNNNNNNNNNNNNNNNNNNNNNNNNNNN | | *FRKPR*EDP*XQEVEAVXSXXD |
| | | NNNNNNNNNNNNNNNNNNNNNNNNNNNNNNNNNNNNNNNNNNNNNNN | | |
| | | NNNNNNNNNNNNNNNNNNNTGCCTGTAGTGCCACTAATTCAGGAAGC | | |
| | | CAAGGTGAGAGGATCCTTGANCCCAGGAGGTGGAGGCTGTAGNGAGC | | |
| | | TGNGANGAT | | |
| prey78527 | 920 | CTGCCCTGTTCACTCTTGCTCATATCTGGACAATTACATCTTCTACT | 921 | CPVHSCSYLDNYIFYCWL*GSLSS |
| | | GTTGGCTGTAAGGATCCCTCAGTTCCCTAAACTATAGCAGTTCCAGC | | LNYSSSSFAYSPKL*NLIFLL*VD |
| | | TTTGCATACTCTCCTAAGCTCTAGAACCTTATTTTTCTTCTATAGGT | | HGHIVSTCLGLSSFYSPTPIENLL |
| | | GGACCATGGACACATAGTCTCAACATGTCTAGGACTGAGCTCATTCT | | FLTVYLI |
| | | ATTCCCCCACTCCCATTGAAAATTTGCTGTTCCTTACTGTATACCTT | | |
| | | ATCTC | | |
| prey78698 | 922 | AAGCGCCCTAGTTCCCAAAGGGCGCTAATGAATGCTGTTTCGTCCTC | 923 | KRPSSQRALMNAVSSSCSPHPITN |
| | | CTGCTCCCCACACCCCATAACTAATAGACTTCAGTTTTTCGGGTTTT | | RLQFFGFF*TEFRSVAXXXXXXXX |
| | | TTTAGACGGAGTTCCGCTCTGTCGCCAGNNNNNNNNNNNNNNNNNNN | | XXXXXXXXXXXXXXXXXXXXXXXX |
| | | NNNNNNNNNNNNNNNNNNNNNNNNNNNNNNNNNNNNNNNNNNNNNNN | | XXXXXXXXXNFNLLYF*IXXXXXX |
| | | NNNNNNNNNNNNNNNNNNNNNNNNNNNNNNNNNNNNNNNNNNNNNNN | | XXXXXXXXXXXXXXXXXXXXXXXX |
| | | NNNNNNNCAATTTTAATTTACTTTATTTTTAAATTNNNNNNNNNNNN | | XXXXXXXXXX |
| | | NNNNNNNNNNNNNNNNNNNNNNNNNNNNNNNNNNNNNNNNNNNNNNN | | |
| | | NNNNNNNNNNNNNNNNNNNNNNNNNNNNNNNNNNNNNNNNNNNNNNN | | |
| | | NNNNNNNNNNNNNNNN | | |
| prey78492 | 924 | TCAGATACAAAATCAAATACATTTTTATATGGCAGCAATAAACAGAC | 925 | SDTKSNTFLYGSNKQTIYTIYPFI |
| | | TATATACACTATATACCCATTTATTTAATAACTCACAACAAAAATAC | | **LTTKIQRTSE*IF*KDL*DLYL |
| | | AAAGAACCTCGGAGTAAATTTTTTAAAAAGACTTATAAGACCTTTAT | | ENNLNFVKYLNNSKEVSCSEVL*N |
| | | TTAGAAAACAATTTAAACTTTGTAAAATACCTCAATAATAGTAAAGA | | YELSSNLSTDSMQSQIKFIXXXXX |
| | | GGTATCATGTTCAGAGGTATTATAAAACTATGAATTATCTTCAAATT | | XXXXXXXXXXXXXXXXXXXXXXAP |
| | | TATCCACAGATTCAATGCAATCTCAAATAAAATTTATCNNNNNNNNN | | LSXRFRPFSCLHLX |
| | | NNNNNNNNNNNNNNNNNNNNNNNNNNNNNNNNNNNNNNNNNNNNNNN | | |
| | | NNNNNNNNNNNNNNNNNNNNNNNNATGACCTCTGTCTNCTCGTTTCA | | |
| | | GACCATTCTCCTGCCTCCACCTGNTGAG | | |
| prey78494 | 926 | GCGGGGCAAGCTACGTTACATTATGGCAGAAAAAAAGTGCAACTGAC | 927 | AGQATLHYGRKKVQLTYYKEIF*V |
| | | ATACTACAAAGAGATTTTTTAAGTTTAAAAAAAGTTTGGATCTTTTG | | *KKFGSFGFLFFFXGXKXLKNP*L |
| | | GATTTCTTTTTTTTTTTTNGGGCNTTAAGGNCCTAAAAAACCCCTAA | | KXXPPXXXXFFFFXGXXPKXXXGV |
| | | TTAAAANTANCCCCCCCAANTANGNANGNTTTTTTTTTTTTTTTNGG | | FSXXXXSXXXXVPXPKEPPXIXPX |
| | | NCNAANCCCAAAAANCCNGNTNGGGGTTTTTTCCNTTNNCCNCCNTT | | GGXLXKXPKKNRXQRGXXGXLXLX |
| | | CCCNANTTTGNNGGGTCCCANNCCCNAAGGAACCCCCCNTAATTNNN | | XXKNNPXKKTQFF*KFFF |
| | | CCCNNAGGGGGGCNTTTGNTAAAACNNCCNAAAAAAAACCGGNCCCA | | |
| | | AAGGGGGANANGGGGAANCCTNNCCCTTTTNNTTTGNAAAAACAACC | | |
| | | CCNANAAAAAAACCCAATTTTTTTAAAAATTTTTTTTCT | | |
| prey78499 | 928 | GCGGGGCANGCTACGTTACATTATGGNAGANAAAAAGTGCAACTGAC | 929 | AGXATLHYGRXKVQLTYYKEIF*V |
| | | ATACTACAAAGAGATTTTTTAAGTTTAAAAAANGTTTGGATCTTTTG | | *KXFGSFGFLFFFLVXXA(KXPEX |
| | | GATTTCTTTTTTTTTTTTTGGTANTNNCGGCCNTAAAGANNCCTGAA | | XAAXXTXXXXFXFSGSXXXXXXRX |
| | | CNNNNGGCAGCCNCCNNAACANGGGNGNGTTTNTTTTNTTTTTCGGG | | XPFXPLSXXWXXXXPXVXXLXTPX |
| | | CTCANNCCANANAANGNTNNTGCGGGNTNNTCCCTTTCNNCCNCTTT | | XGXFVXPLXKTRVXWGTXGXLXPF |
| | | CTTGNNTNTGGTNNNTNNCCNTCCCCNATGTATNANCNTTGGNCACC | | XXXNXAPPXXXFF |
| | | CCCCNANGGGGGNCNTTTGTNAANCCCCTTCANAAAACNCGGGTNAA | | |
| | | NTGGGGGACANGGGGNANCCTGGNCCCTTTTNGNNTGNAAAATAGNG | | |
| | | CCCCCCCCANANCCCNTTTTTTTTT | | |
| prey78501 | 930 | TCCAATGCTCAATATAGGACAAAAAAGAACAGGAAAGGAGAAAATAT | 931 | SNAQYRTKKKRKGENMKECILVNS |
| | | GAAAGAATGTATATTAGTTAACTCAGATTTTTGGATCTGGGTGGATT | | DFWIWVDFGWC*IGLGTSLLFFLI |
| | | TCGGATGGTGTTAAATTGGCCTGGGGACTTCTTTATTATTTTTTTTA | | VALVCNIHGLA*MT*RIYFLI*RG |
| | | ATCGTAGCATTGGTTTGTAATATTCATGGTTTAGCCTGAATGACTTA | | LKEHEQ*TCQ*LF*NINR*LFVLL |
| | | AAGGATTTATTTTTTAATTTAACGNGGGTTAAAAGAACATGAGCAAT | | KGHIEDSRAHDSGWLFFLEE*FSQ |
| | | AGACCTGCCAATAACTATTTTAAAATATAAACAGATAACTGTTTGTT | | VELNTSYATV |
| | | TTGCTCAAAGGTCACATTGAGGATTCACGAGCTCATCACTCAGGATG | | |
| | | GCTATTTTTCTTAGAGGAATGATTTAGTCAAGTTGAATTGAATACCT | | |
| | | CCTACGCCACTGTC | | |
| prey78736 | 932 | GGGAGCAGTTATAATTATACAGTATATGCCGTTTTATAACTTAACTT | 933 | GSSYNYTVYAVL*LNFFI*HGFIN |
| | | TTTCATATAACATGGCTTTATAAACTTTTTCTGAAAACTTCTTAAGC | | FF*KLLKHNCPISY*IIVKWMCNY |
| | | ATAATTGTCCAATTAGCTACTAAATAATTGTTAAATGGATGTGCAAC | | *LHQFLTLKM*VLSKFLLFSITPP |
| | | TATTGACTTCACCAATTCCTTACTTTAAAAATGTAGGTGCTTTCCAA | | VSSLGIKLFLDFILFCQSLPGAGS |
| | | GTTTTTGCTCTTTTCAATAACACCACCGGTATCCTCTTTGGGAATAA | | TEEEAEALPTLLADLGNCFPFPSG |
| | | AGCTATTTCTAGATTTCATACTATTTTGTCAATCCCTTCCAGGAGCA | | VSSQLCGSP |
| | | GGCTCAACAGAGGAGGAGGCAGAAGCATTGCCAACGCTCTTAGCAGA | | |
| | | TCTGGGCAACTGTTTCCCCTTCCCCTCAGGAGTCTCCTCTCAACTGT | | |
| | | GTGGGAGCCCAC | | |
| prey70344 | 934 | TGCAAACCCAAAGACTAAAGATTATGGAATATTATGAGAAGAAAGAG | 935 | QTQRLKIMEYYEKKEKQIEQQKKI |
| | | AAACAGATTGAGCAGCAGAAGAAAATTCAGATGTCCAATTTGATGAA | | QMSNLMNQARLKVLRARDDLITDL |
| | | TCAAGCGAGACTCAAAGTCCTCAGAGCAAGAGATGACCTTATCACAG | | LNEAKQRLSKVVKDTTRYQVLLDG |
| | | ACCTACTAAATGAAGCAAAACAGAGACTCAGCAAGGTGGTAAAAGAT | | LVLQGLYQLLEFRNIVRCRKQDFP |
| | | ACAACCAGGTACCAAGTGCTGCTGGATGGACTGGTTCTCCAGGGTTT | | LVKAAVQKAIPMYKIATKNDVDVQ |
| | | GTACCAGTTGCTGGAGCCCCGAATGATTGTTCGTTGCAGGAAACAAG | | IDQESYLPEDIAGGVEIYNGDRKI |
| | | ATTTCCCTCTGGTAAAGGCTGCAGTGCAGAAGGCAATTCCTATGTAC | | KVSNTLESRLDLIAQQMMPEVRGA |
| | | AAAATTGCCACCAAAAACGATGTTGATGTCCAAATTGACCAGGAGTC | | LFGANANRKFLD* |
| | | CTACCTGCCTGAAGACATAGCTGGTGGAGTTGAGATCTATAATGGAG | | |
| | | ATCGTAAAATAAAGGTTTCCAACACCCTGGAAAGCCGGCTGGATCTC | | |
| | | ATAGCCCAGCAGATGATGCCAGAAGTCCGGGGAGCCTTGTTTGGTGC | | |
| | | AAATGCCAACAGGAAGTTTTTGGACTAA | | |
| prey78572 | 936 | TTTGTTGTGGGGGGCTGTCCTGTGCATCGTAGGATGCTTAGCATCTG | 937 | FVVGGCPVHRRMLSICSVDANDIT |
| | | CTCAGTAGATGCCAATGATATTATCTCAGATGTGACAACCAAAATGT | | SDVTTKMSPXXAKCSWKTKYTLYS |
| | | CTCCTANTTNTGCCAAATGTTCCTGGAAGACAAAATATACCCTCTAC | | SV*NYCTRISAF*N*LNGYTFSSH |
| | | TCCAGTGTATAGAATTACTGTACTAGAATATCAGCTTTTTGAAATTG | | LLICSWNFKSVVFDLLGRW*LLKI |
| | | ATTAAATGGTTATACTTTCTCTTCCCATCTGCTGATTTGTAGCTGGA | | AGS*AYPTPTESXTLKVEPRXLIE |
| | | ATTTTAAAAGTGTGGTCTTTGACTTACTTGGNAGGTGGTAGCTTCTT | | TRHYLPTPX |
| | | AAAATTGCAGGTTCCTGAGCTTATCCTACACCTACTGAATCANAAAC | | |
| | | GCTGAAGGTAGAACCCAGANATCTGATTTTTACAAGACACTACCTTC | | |
| | | CCACCCCCAGNN | | |
| prey78577 | 938 | GTGANTNCGCGGNCCTANNTGGCACCGTGGANGGCCGTGAGCGACAC | 939 | VXXRXXXAPWXAVSDTRTPGXKXX |
| | | TAGGACCCCAGGGTTNAAAANNGNCNGANCAAATGGAAAGCTCACCG | | XXNGKLTVLPXXVXXXGXHPPXGP |
| | | TGCTACCTGNNNANGTGGNATTNTNNGGNCNACATCCNCCTNGNGGA | | CGWXGPGXTXISNXXXXXCXVSPA |
| | | CCCTGTGGATGGNGTGGTCCTGGTGNAACCTNAATATCTAATANAGN | | PXXIXXXXXXLLDLTFHKDLXXAX |
| | | GNNGAGNNTATGTNACGTCTCNCCTGCCCCTTNNNNTATTNCCGNAA | | XQENPTDPXGXXPP*HQMRERLIK |
| | | NGANCTGNATNCTCCTGGACCTGACCTTTCACAAGGACCTGNTNNNN | | IXXPSTLTLSPX |
| | | GCCNNCGNACAGGAAAATCCNACCGATCCTCNNGGACNANANCCNCC | | |
| | | TTGACACCAGATGAGGGAACGNCTTATCAAGATTANTGNCCCAAGCA | | |
| | | CGCTTACCCTTTCACCTAGN | | |
| prey78599 | 940 | CCAGTCCAACGCCCAGAAGATAATCCCACCTCTGTTCAGCTACCGCC | 941 | QSNAQKIIPPLFSYRLAQQQLKEM |
| | | TTGCCCAGCAGCAGTTAAAGGAGATGAAAAAGAAAGGTCTGACGGAA | | KKKGLTETTKVYHVSQSPLTDTAI |
| | | ACTACCAAAGTGTATCACGTGTCTCAGAGTCCCCTGACAGACACTGC | | DAAPSAPLDLESPEEAAANKFRIK |
| | | CATTGATGCGGCCCCCAGCGCTCCCTTAGATTTGGAAAGCCCGGAAG | | SPFPEQPAVSAGERPPSRLDLNVT |
| | | AAGCTGCAGCAAACAAGTTCCGGATCAAATCCCCATTTCCGGAGCAG | | QASCAISPSQTLIRKSQARHVGSR |
| | | CCCGCGGTCAGTGCCGGGGAAAGGCCTCCCTCCAGGCTGGATCTAAA | | GGPSERSHARN |
| | | TGTGACTCAGGCCAGTTGTGCCATAAGCCCCAGCCAGACTCTGATCC | | |
| | | GCAAGTCACAGGCAAGGCACGTGGGCAGCAGAGGGGGCCCGTCCGAA | | |
| | | AGGAGCCATGCCAGGAAC | | |
| prey78607 | 942 | GGCCGAGATTTCCTACGAAGAGGCCCTGAGGAGGGCCCGGCGCGGTC | 943 | AETSYEEALRRARRGRRENVGLYP |
| | | GCCGGGAGAATGTGGGGCTGTACCCCGCGCCTGTGCCTCTGCCCTAC | | APVPLPYASPYAYVASDSEYSAEC |
| | | GCCAGCCCCTACGCCTACGTGGCTAGCGACTCCGAGTACTCGGCCGA | | ESLFHSTVVDTSEDEQSNYTTNCF |
| | | GTGCGAGTCCCTGTTCCACTCCACCGTGGTGGACACCAGTGAGGACG | | GDSESSVSEGEFVGESTTTSDSEE |
| | | AGCAGAGCAATTACACCACCAACTGCTTCGGGGACAGCGAGTCGAGT | | SGGLIWSQFVQTLPIQTVTAPDLH |
| | | GTGAGCGAGGGCGAGTTCGTGGGGGAGAGCACAACCACCAGCGACTC | | NHPAKTFVKIKASHNLKKKILRFR |
| | | TGAAGAAAGCGGGGGCTTAATTTGGTCCCAGTTTGTCCAGACTCTGC | | SGSLKLNTTV* |
| | | CCATTCAAACGGTAACGGCCCCAGACCTTCACAACCACCCCGCAAAA | | |
| | | ACCTTTGTCAAAATTAAGGCCTCACATAACCTCAAGAAGAAGATCCT | | |
| | | CCGCTTTCGGTCTGGCTCTTTGAAACTGATGACGACGGTTTGA | | |
| prey56846 | 944 | GAAGATTGTAAATGCCAAGAAAGATGTTGTGAACACAAAGATGTTTG | 945 | KIVNAKKDVVNTKMFEELKSRLDT |
| | | AGGAGCTCAAGAGCCGTCTGGACACCCTGGCCCAGGAGGTGGCCCTG | | LAQEVALLKEQQALQTVCLKGTKV |
| | | CTGAAGGAGCAGCAGGCCCTGCAGACGGTCTGCCTGAAGGGGACCAA | | HMKCFLAFTQTKTFHEASEDCISR |
| | | GGTGCACATGAAATGCTTTCTGGCCTTCACCCAGACGAAGACCTTCC | | GGTLSTPQTGSENDALYEYLRQSV |
| | | ACGAGGCCAGCGAGGACTGCATCTCGCGCGGGGGCACCCTGAGCACC | | GNEAEIWLGLNDMAAEGTWVDMTG |
| | | CCTCAGACTGGCTCGGAGAACGACGCCCTGTATGAGTACCTGCGCCA | | ARIAYKNWETEITAQPDGGKTENC |
| | | GAGCGTGGGCAACGAGGCCGAGATCTGGCTGGGCCTCAACGACATGG | | AVLSGAANGKWFDKRCRDQLPYIC |
| | | CGGCCGAGGGCACCTGGGTGGACATGACCGGCGCCCGCATCGCCTAC | | QFAGIV* |
| | | AAGAACTGGCAGACTGAGATCACCGCGCAACCCGATGGCGGCAAGAC | | |
| | | CGAGAACTGCGCGGTCCTGTCAGGCGCGGCCAACGGCAAGTGGTTCG | | |
| | | ACAAGCGCTGCCGCGATCAGCTGCCCTACATCTGCCAGTTCGGGATC | | |
| | | GTGTAG | | |
| prey78627 | 946 | GAGAAGGAAGGAAGGAGAAGGCAGGCAGGAAATCAATATGACAGCAT | 947 | EKEGRRRQAGNQYDSIALELRTHL |
| | | AGCATTGGAGCTAAGAACACACCTTCTAGAAGCAGACTCTATGAATT | | LEADSMNSNPGSTIYKLYFPL*NM |
| | | CAAATCCTGGGTCTACCATTTATAAGCTGTACTTTCCTCTGTAAAAT | | NLQYLLLTDCYKDEMC*YR*SKHS |
| | | ATGAATCTACAGTACCTACTTCTCACAGATTGTTATAAAGATGAGAT | | VNMRH*PEPNKVPSSNIIIECITF |
| | | GTGTTAATATAGATAAAGTAAGCACTCAGTGAATATGAGGCATTAAC | | FSLQSKFTYSISFGSHKTP*ELAK |
| | | CAGAACCCAACAAGGTGCCATCATCAAATATAATAATTGAGTGCATA | | YFPPFSLKL |
| | | ACTTTTTTCAGTTTACAAAGCAAATTTACATATAGCATTTCTTTTGG | | |
| | | ATCTCACAAAACTCCATGAGAGCTGGCAAAATACTTCCCCCCATTTT | | |
| | | CTTTGAAGCTCAT | | |
| prey78635 | 948 | TGTCCATATATACGGGATCCTTTAAATGGCCAAGCAGTCCCTGCAAA | 949 | CPYIRDPLNGQAVPANGTYEFCYQ |
| | | TGGGACTTACGAGTTTGGTTATCAGATGCACTTTATTTGTAATGAGG | | MHFICNEGYYLIGEEILYCELKGS |
| | | GTTATTACTTAATTGGTGAAGAAATTCTATATTGTGAACTTAAAGGA | | VAIWSGKPPICEKVLCTPPPKIKN |
| | | TCAGTAGCAATTTGGAGCGGTAAGCCCCCAATATGTGAAAAGGTTTT | | GKHTFSEVEVFEYLDAVTYSCDPA |
| | | GTGTACACCACCTCCAAAAATAAAAAATGGAAAACACACCTTTAGTG | | PGPDPFSLIGESTIYCGDNSVWSR |
| | | AAGTAGAAGTATTTGAGTATCTTGATGCAGTAACTTATAGTTGTGAT | | AAPECKVVKCRFPVVENGKQISGF |
| | | CCTGCACCTGGACCAGATCCATTTTCACTTATTGGAGAGAGCACGAT | | GKKFYYKATVMFECDKGFYLDGSD |
| | | TTATTGTGGTGACAATTCAGTGTGGAGTCGTGCTGCTCCAGAGTGTA | | TIVCDSNSTWDPPVPKCLKGPRPT |
| | | AAGTGGTCAAATGTCGATTTCCAGTAGTCGAAAATGGAAAACAGATA | | YKPPVSNYPGYPKPEEGILDSLDV |
| | | TCAGGATTTGGAAAAAAATTTTACTACAAAGCAACAGTTATGTTTGA | | WVIAVIVIAIVVGVAVICVVPYRY |
| | | ATGCGATAAGGGTTTTTACCTCGATGGCAGCGACACAATTGTCTGTG | | LQRRKKKGKADGGAEYATYQTKST |
| | | ACAGTAACAGTACTTGGGATCCCCCAGTTCCAAAGTGTCTTAAAGGT | | TP |
| | | CCTAGGCCTACTTACAAGCCTCCAGTCTCAAATTATCCAGGATATCC | | |
| | | TAAACCTGAGGAAGGAATACTTGACAGTTTGGATGTTTGGGTCATTG | | |
| | | CTGTGATTGTTATTGCCATAGTTGTTGGAGTTGCAGTAATTTGTGTT | | |
| | | GTCCCGTACAGATATCTTCAAAGGAGGAAGAAGAAAGGGAAAGCAGA | | |
| | | TGGTGGAGCTGAATATGCCACTTACCAGACTAAATCAACCACTCCA | | |
| prey78644 | 950 | GCGGGGCAAGCTACGTTACATTATGGCAGAAAAAAAGTGCAACTGAC | 951 | AGQATLHYGRKKVQLTYYKEIF*V |
| | | ATACTACAAAGAGATTTTTTAAGTTTAAAAAAAGTTTGGATCTTTTG | | *KKFGSFGFLFFFXGXXGXKNX*X |
| | | GATTTCTTTTTTTTTTTTNGGGCNTTANGGGNCNAAAAAACCNNTAA | | KXXPXXKXXFFFFXGXPQXXXGGX |
| | | TNAAAANNANCCCCCCNAAANAGGNNGGNTTTTTTTTTTTTTTTNGG | | FXXXXXSGXXGVXXPKXPPXXXPX |
| | | NCNACCCCAAANAANCNGGGGNGGCCNTTTTNCCNTTNCCCNNCNTT | | GXPXXXXPXQXRXXXGXXGNPXPF |
| | | CCGGNNTTNGNGGGGTCCNNNNCCCAAAGNNGCCCCCCTTNNTTNNC | | XLEXRXPXXKP |
| | | CCCNANGGGGNNCCNTTNGNNAANNCNCCNGANCAAANCCGNNNGNN | | |
| | | AANGGGGGANNAGGGGAACCCTNTNCCTTTTNGNTTGGAAAANCGGG | | |
| | | NCCCNNNNNAAAAACCC | | |
| prey32308 | 952 | ATGTACGAGCAACTCAAGGGCGAGTGGAACCGTAAAAGCCCCAATCT | 953 | MYEQLKGEWNRKSPNLSKCGEELG |
| | | TAGCAAGTGCGGGGAAGAGCTGGGTCGACTCAAGCTAGTTCTTCTGG | | RLKLVLLELNFLPTTGTKLTKQQL |
| | | AGCTCAACTTCTTGCCAACCACAGGGACCAGCTGACCAAACAGCAGC | | ILARDILEIGAQWSILRKDIPSFE |
| | | TAATTCTGGCCCGTGACATACTGGAGATCGGGGCCCAATGGAGCATC | | RYMAQLKCYYFDYKEQLPESAYMH |
| | | CTACGCAAGGACATCCCCTCCTTCGAGCGCTACATGGCCCAGCTCAA | | QLLGLNLLFLLSQNRVAEFHTELE |
| | | ATGCTACTACTTTGATTACAAGGAGCAGCTCCCCGAGTCAGCCTATA | | RLPAKDIQTNVYIKHPVSLEQYLM |
| | | TGCACCAGCTCTTGGGCCTCACCTCCTCTTCCTGCTGTCCCACAACC | | FGSYNKVFLAKGNIPAESYTFFID |
| | | GGGTGGCTGAGTTCCACACGGAGTTGGAGCGGCTGCCTGCCAAGGAC | | ILLDTIRDEIAGCIEKAYEKILFT |
| | | ATACAGACCAATGTCTACATCAAGCACCCAGTGTCCCTGGAGCAATA | | EATRILFFNTPKKMTDYAKKRGWV |
| | | CCTGATGGAGGGCAGCTACAACAAAGTGTTCCTGGCCAAGGGTAACA | | LGPNNYYSFASQQQKPEDTTIPST |
| | | TCCCCGCCGAGAGCTACACCTTCTTCATTGACATCCTGCTCGACACT | | ELAKQVIEYARQLEMIV* |
| | | ATCAGGGATGAGATCGCTGGGTGCATCGAGAAGGCCTACGAGAAAAT | | |
| | | CCTTTTCACTGAGGCCACCCGGATCCTCTTCTTCAACACACCCAAAA | | |
| | | AGATGACAGACTACGCCAAGAAGCGAGGGTGGGTCCTGCGCCCCAAC | | |
| | | AACTACTACAGTTTTGCCAGCCAGCAGCAGAAGCCGGAAGACACCAC | | |
| | | CATTCCCTCCACAGAACTGGCCAAACAGGTCATCGAGTATGCCCGGC | | |
| | | AGCTGGAGATGATCGTCTGA | | |
| prey78679 | 954 | ACTGCCCACACCTGTAGTAGAACAAGTTGCACAAATAAATGATAATT | 955 | LPTPVVEQVAQINDNSELELTSVV |
| | | CAGAACTCGAGTTGACATCAGTTGTGGAAAATACTTTTCCAGCACAA | | ENTFPAQDIVHTVTVKRKRCKSQP |
| | | GATATTGTGCACACTGTTACAGTGAAACCGAAACGTGGAAAATCACA | | NCALKEHSMSNIASVKSPYEAENS |
| | | GCCAAACTGTGCTCTGAAAGAACACTCTATGTCTAATATAGCCAGCG | | GEELDQRYSKAKPMCNTCGKVFSE |
| | | TCAAGAGTCCTTATGACGCGGAGAACTCCGGGGAAGAGCTGGATCAG | | ASSLRRHMRIHKGVKPYVCHLCGK |
| | | AGGTATTCCAAGGCCAAGCCAATGTGTAACACATGTGGGAAAGTGTT | | AFTQCNQLKTHVRTHTGEKPYKCE |
| | | TTCAGAAGCCAGCAGTTTGAGAAGGCACATGAGAATACATAAAGGAG | | LCDKGFAQKCQLVFHSRMHHGEEK |
| | | TCAAACCTTACGTCTGCCACTTATGTGGAAAGGCATTTACCCAATGT | | PYKCDVCNLQFATSSNLKIHARKH |
| | | AACCAGCTGAAAACGCATGTAAGAACTCATACAGGTGAGAAGCCATA | | SGEKPYVCDRCGQRFAQASTLTYH |
| | | CAAATGTGAATTGTGTGATAAAGGATTTGCTCAGAAATGTCAGCTAG | | VRRHTGEKPYVCDTCGKAFAVSSS |
| | | TCTTCCATAGTCGCATGCATCATGGTGAAGAAAAACCCTATAAATGT | | LITHSRKHTGEKPYICGICGKSFI |
| | | GATGTATGCAACTTACAGTTTGCAACTTCTAGCAATCTCAAGATTCA | | SSGELNKHFRSHTGERPFICELCG |
| | | TGCAAGGAACCATAGTGGAGAGAAGCCATATGTCTGTGATAGGTGTG | | NSYTDIKNLKKHKTKVHSGADKTL |
| | | GACAGAGATTTGCTCAAGCCAGCACACTGACCTATCATGTCCGTAGG | | DSSAEDHTLSEQDSIQKSPLSETM |
| | | CATACTGGAGAAAGCCTTATGTATGTGATACCTGTGGGAAGGCATTT | | DVKPSDMTLPLALPLGTEDHHMLL |
| | | GCTGTCTCTAGTTCTCTTATCACTCATTCTCGAAAACATACAGGTGA | | PVTDTQSPTSDTLLRSTVNGYSEP |
| | | AAAACCATACATATGTGGTATTTGTGGGAAAAGTTTTATTTCCTCAG | | QLIFLQQLY* |
| | | GAGAGCTCAACAAACACTTTCGGTCCCATACAGGAGAAAGACCATTT | | |
| | | ATCTGCGAATTATGTGGAAATTCTTACACAGATATTAAAAATTTAAA | | |
| | | GAACCACAAAACAAAAGTCCATTCTGGTGCAGATAAAACTCTAGACT | | |
| | | CCACTGCAGAGGATCATACTTTGAGTGACAGGATTCCATACAAAAAA | | |
| | | GTCCTTTATCAGAAACTATGGATGTGAAGCCTTCTGATATGACTTTA | | |
| | | CCATTAGCTCTTCCACTTGGGACTGACGACCATCACATGCTTCTGCC | | |
| | | TGTCACGGATACTCAGTCTCCTACATCAGATACATTGTTGAGGTCAA | | |
| | | CTGTGAATGGGTATTCAGACCACAGTTGATTTTTTTACAACAATTAT | | |
| | | ACTGA | | |
| prey78662 | 956 | TGGGGCAAAGGGGAGGAAGGGAGGCAGGAAAGAAGATTTACACCCAA | 957 | WGKGEEGREERRFRPKTNPXSFSP |
| | | AACTAACCCANCATCATTTTCTCCNGCTNAGATTCAATATAANAAAG | | AXIQYXKGLTXXVQLLRGVVAYCI |
| | | GACTAACACANGNAGTACATTATTGAGAGGTGTTGTGGCTTATGGTA | | F*FSYIKHCLFIFLCFKNTSXKAA |
| | | TTTTTTAATTTTCCTACATTAAACATTGTTTATTTATATTTTTAGGG | | XYY*RNC*PYTKYLSVKNYN*VAF |
| | | TTTAAAAATACTTCCNAAAAAGCAGCTNATTATTATTACAGGAACTG | | LXLCSVKHISXEMCIFLFYTPHKQ |
| | | TTAGCCTTATACTAAATATTTATCAGTTAAAAATTATAATTAAGTAG | | GCEVK*IWEKCKVAKAFKMLIPAR |
| | | CATTTCTCANATTGTGTTCTGTGAAACATATTTCTTGNGAAATGTGC | | XXXXXXXXXXXXXXXXXXXXXXXX |
| | | ATATTTCTTTTTTATACTCCCCATAAACAGGGATGTGAGGTCAAATA | | XXXXXXXXXXXXXXXXXXXXXXXG |
| | | AATTTGGGAAAAATGCAAAGTATAAAAAGCATTTAAAATGCTNATAC | | HXXSPPPGRPRX*IA*TWEEDV |
| | | CGGCCAGGCANNNNNNNNNNNNNNNNNNNNNNNNNNNNNNNNNNNNN | | |
| | | NNNNNNNNNNNNNNNNNNNNNNNNNNNNNNNNNNNNNNNNNNNNNNN | | |
| | | NNNNNNNNNNNNNNNNNNNNNNNNNNNNNNNNNNNNNNNNNNNNNNN | | |
| | | NNNNNNNNNNTGGACACNTGTNGTCCCCGCCACCTCGGACGCCGAGA | | |
| | | TCNTGAATCGCTTGAACCTGGGAGGAAGACGTTGN | | |
| prey78664 | 958 | TGGTACTTCATGGCCAACTTGTGAAAGGCCTTCTTGATTTGGCGCTC | 959 | WYFMANL*KAFLIWRSDADFGTPK |
| | | TGATGCCGATTTTGGCACACCTAAAATATCATAGTAGCTTTTTGAGC | | IS**LFEARINSVII*NANCKDEN |
| | | CCAGAATTAATTCTCTTATCATATAATGCAAATTGCAAAGATCAAAA | | *LGSSHF*YPKTEKKKKCYGWS*S |
| | | TTGACTGGGGACTAGCCATTTCTAATATCCTACAGAAAGAAAAAAAA | | *NVFLLKEIQHKVNLMQYQNSKHT |
| | | ATGTTATGGATGGACCTAATCTTAAAACGTTTTTCTTTTAAAAGAAT | | VSITCPHVISLLVL*LSNWLNLIE |
| | | CCAACACAAGCTAAACCTAATGCAGTATCAAAATAGTAAGCATACAG | | NKNNSWLCLSTNTLA |
| | | TATCTATTACCTGTCCACATGTAATTTCACTTTTAGTTTTATAACTC | | |
| | | TCAACTCGCTTAACCTTATTGACACAAAATAATTCTTGGCTTTGTCT | | |
| | | GTCTACCAATACTCTGGCCAT | | |
| prey78669 | 960 | ATACCCTNTATGGNNGGCTCCTGTGNAGANATCTGANGACGTNCNAG | 961 | IPXMXGSCXXI*XRXRX*WHXWRS |
| | | GANTTGATGGCATTNNTGGCGNTCAGTCAGNGCACNNTAACTTCCAT | | VXAX*LA*XIGXXHDXHXLYXXXE |
| | | AACNCATAGGNNATATNCATGATNCNCACNCATTGTATCNATNATAN | | *XGGICLVTSYLLILYXXXXTFFF |
| | | GAATAANGTGGCGGCATTTGTTTGGTAACTTCATACCTTCTNATTCT | | LXXXAXSSXPXXDSHXCAXCSXLH |
| | | CTATTNNGNGNNCANTACATTTTTTTTTCTTTNAANTTNTGCCNACT | | HXXHFFIXAIXLLPXXAVEYNXX* |
| | | CTTCCNNNCCGNAAAANGACTCTCATNACTCTGCCNTNTGTTCAANG | | PXXXXCYXXXXFINR |
| | | CTGCACCACNNNNTTCATTTCTTCATANCTCCTATTNATCTCCTTCC | | |
| | | TNANNCAGCCCTTGAATACAATNGNANATGACCTCNCNNCNTNNGCT | | |
| | | GCTATNANNCATANNCCTTTATTAACCGCT | | |
| prey78672 | 962 | CNNNNNNNNNNNNNNNNNNNNNNNNNNNNNNNNNNNNNNNNNNNNNN | 963 | XXXXXXXXXXXXXXXXXXXXXXXX |
| | | NNNNNNNNNNNNNNNNNNNNNNNNNNNNNNNNNNNNNNNNNNNNNNN | | XXXXXXXXXXX*NYGHALPGLANF |
| | | NNNNNNNNNNGTAGAACTACCCGCATGCACTACCAGGCCTGGCTAAT | | LXXXXXXXXXXXXXXXXXXXXXXX |
| | | TTTTTAATNNNNNNNNNNNNNNNNNNNNNNNNNNNNNNNNNNNNNNN | | XXXXXXXXXXXXXXXXXXXXXXVE |
| | | NNNNNNNNNNNNNNNNNNNNNNNNNNNNNNNNNNNNNNNNNNNNNNN | | KQHSWHIRTTQVNKTGDQINKGKP |
| | | NNNNNNNNNNNNNNNNNNNNNNNNNNNNNNNNNNNNNNNNNNNNNNA | | FCDTVPQAT |
| | | GTGGAGAAGCAGCACTCTTCGCACATAAGGACAACACAAGTCAACAA | | |
| | | GACTGGGGACCAAATTAACAAAGGGAAGCCTTTCTGTGATACAGTCC | | |
| | | CCCAAGCCACCGC | | |
| prey78674 | 964 | ATTACCCTTNATGGAGGGCTCNNGNGAANATTAATGAANCCATGGTT | 965 | ITLXGGLXXXLMXPWXEXEGINXG |
| | | NGAGNTTGAGGGCATTAATTGNGGCNCANGGAGNGCACANTNAATTG | | XXXAXXIAXXIGXIQINXIVXIIX |
| | | CATANNACATAGGTNATATACAAATAAATATNATTGTANTTATAATA | | XXXXXFXVXPXXSFXILXSLXXFF |
| | | ANAANAAANTGNCANNTTTTTNTGGTANCTCCAGNCTNATCATTCTN | | FXLXYSPXXWKXX*XXXIFQCCTT |
| | | TATTCTGTGNTCNTTANATGNTTTTTTCTTTTGNCTTTNGTACTCTC | | XLQVALTPNDLXF*TAXAFNXHIT |
| | | CTNCGANCTGGAAAANGNTCTGANTTNNNNCCATCTTTCAATGCTGT | | XXXFXAILTY |
| | | ACCACAAGNCTNCAGGTGGCTCTAACTCCTAATGATCTCTNCTTCTA | | |
| | | AACNGCCNTTGCATTTAATGNGCATATTACCGNTNCNNCNTTTGNTG | | |
| | | CTATTCTCACATATGC | | |
| prey78675 | 966 | GCCAGCCAGCGTCACCCTCTTCTCCAATTTGTAGGTGTATTTTATGC | 967 | ASQRHPLLQFVCVFYALKASSSPC |
| | | TCTTAAGGCTTCATCTTCTCCCTGTAACTGAGATTTCTACCACACCT | | N*DFYHTFEQCSFPSGYLKTVLKE |
| | | TTGAACAATGTTCTTTCCCTTCTGGTTATCTGAAGACTGTCCTGAAA | | DISVVISRSFLVDHISXGX*XNXL |
| | | GAAGACATAAGTGTTGTGATTAGTACAAGCTTTCTAGTAGACCATAT | | VAPFYFVPVXGKPIFELLIXLALX |
| | | TTCTTNTGGANTGTAANNAAATTGNTTAGTAGCNCCTTTTTACTTTG | | SGXLEKACXHXX*VX**KFRYKGA |
| | | TNCCTGTCNNTGGAAAGCCNATTTTTGAATTGCTGATTNCTTTGGCT | | L*TVELC*EHXSSTTQDL*KYELA |
| | | TTAANNAGTGGTCNCCTAGAAAAAGCTTGTANNCATANCNCATGAGT | | KLNSANRFSCVFSEPPQD*TKCFL |
| | | ANTCTGATAAAAGTTCAGATACAAAGGAGCACTGTAAACTGTAGAGC | | FGHPEEVVLAVNAL*V*KKS***N |
| | | TATGTTAAGAGCATTGNAGTAGTACAACCCAAGATTTGTAGAAGTAT | | LVFDTVHELSS*IFTYSERNSGKG |
| | | GAGTTGGCGAAATTAAATAGTGCAAATAGATTTAGTTGTGTTTTCTC | | ILVMTKSLQM*LSHYKCD*VITVD |
| | | AGAGCCGCCACAGATTGAACAAAATGTTTTCTGTTTGGGCATCCTGA | | PLGALKRIRFISRFQRTWNNNKLW |
| | | GGAAGTTGTATTAGCTGTTAATGCTCTGTGAGTTTAGAAAAAGTCTT | | ILHSV*LDFGTLASVLFTGIGIRV |
| | | GATAGTAAAATCTAGTTTTTGACACAGTGCATGAACTAAGTAGTTAA | | HRQSS*SHTTGDLTKLVSFLCS*A |
| | | ATATTTACATATTCAGAAAGGAATAGTGGAPAAGGTATCTTGGTTAT | | LPFFF*FGACNFLNNDSTSWAITL |
| | | GACAAAGTCATTACAAATGTGACTAAGTCATTACAAATGTGACTGAG | | *CSWPLFCCCFAILKLRPSTNVGC |
| | | TCATTACAGTGGACCCTCTGGGTGCATTGAAAAGAATCCGTTTTATA | | *KGFCSFNMQL*GMEVSSFLSCVV |
| | | TCCAGGTTTCAGAGGACCTGGAATAATAATAAGCTTTGGATTTTGCA | | SRCGV*GSESCKFKFMIR*RVDIE |
| | | TTCAGTGTAGTTGGATTTTGGGACCTTGGCCTCAGTGTTATTTACTG | | IVLFPDQKMNKAFLNKIQTFNQVL |
| | | GGATTGGCATACGTGTTCACAGGCAGAGTAGTTGATCTCACACAACG | | ICMTEKKYTTSR |
| | | GGTGATCTCACAAAACTGGTAAGTTTCTTATGCTCATGAGCCCTCCC | | |
| | | TTTTTTTTTTTAATTTGGTGCCTGCAACTTTCTTAACAATGATTCTA | | |
| | | CTTCCTGGGCTATCACATTATAATGCTCTTGGCCTCTTTTTTGCTGC | | |
| | | TGTTTTGCTATTCTTAAACTTAGGCCAAGTACCAATGTTGGCTGTTA | | |
| | | GAAGGGATTCTGTTCATTCAACATGCAACTTTAGGGAATGGAAGTAA | | |
| | | GTTCATTTTTAAGTTGTGTTCTCAGTAGGTGCGGTGTCTAGGGTAGT | | |
| | | GAATCCTGTAAGTTCAAATTTATGATTAGGTGACGAGTTGACATTGA | | |
| | | GATTGTCCTTTTCCCTGATCAAAAAATGAATAAAGCCTTTTTAAACA | | |
| | | AAATCCAAACTTTTAATCAAGTCTTGATATGTATGACTGAGAAAAAA | | |
| | | TACACTACATCTAGA | | |
| prey69554 | 968 | CTTCCGGGAGGATTTCCATTATAATGACACAGCTGGGTACTTCATTA | 969 | FREDFHYNDTAGYFIIGGSRYVAG |
| | | TTGGAGGGAGCAGGTATGTGGCTGGCATTGAAGGGTTTTTTGGACCC | | IEGFFGPLKYYRLRSLHPAQIFNP |
| | | CTGAAGTACTATCGCCTTCGCAGTCTGCACCCCGCCCAGATTTTTAA | | LLEKQLAEQTKLYYERCAEVQAIV |
| | | TCCCCTCCTTGAGAAGCAACTTGCTGAACAAATCAGTTATATTATGA | | SVYASAAKHGGERQEACHLHNSYL |
| | | AAGGTGTGCTGAGGTTCAAGAAATAGTATCTGTGTATGCATCTGCAG | | DLQRRYGRPSMCRAFPWEKELKDK |
| | | CAAAGCACGGGGGCGAGAGACAAGAAGCATGCCACCTCCACAACTCC | | HPSLFQALLEMDLLTVPRNQNESV |
| | | TACCTGGACCTCCAGCGCAGGTATGGGAGACCCTCGATGTGCAGAGC | | SEIGGKIFEKAVKRLSSIDGLHQI |
| | | CTTCCCCTGGGAGAAGGAGCTGAAAGACAAACACCCCAGCTTGTTCC | | SSIVPFLTDSSCCGYHKASYYLAV |
| | | AGGCATTGCTGGAGATGGATCTGCTGACCGTGCCAAGGAACCAAAAT | | FYETGLNVPRDQLQGMLYSLVGGQ |
| | | GAATCTGTATCAGAAATCGGTGGGAAGATATTTGAGAAGGCTGTAAA | | GSERLSSMNLGYKHYQGIDNYPLD |
| | | GAGACTCTCTAGCATTGATGGTCTTCACCAAATTAGCTCTATCGTCC | | WELSYAYYSNIATKT |
| | | CCTTTCTGACGGATTCCAGCTGCTGTGGATACCATAAAGCATCCTAC | | |
| | | TACCTTGCAGTCTTTTATGAGACTGGATTAAATGTTCCTCGGGATCA | | |
| | | GCTGCAGGGCATGTTGTATAGTTTGGTTGGAGGCCAGGGGAGTGAGA | | |
| | | GGCTGTCTTCAATGAATCTTGGGTATAAACACTACCAGGGTATTGAC | | |
| | | AACTACCCCCTGGACTGGGAACTGTCGTATGCCTACTACAGCAACAT | | |
| | | TGCCACCAAGACACC | | |
| prey2530 | 970 | ATGGGCGACAAAGGGACCCGAGTGTTCAAGAAGGCCAGTCCAAATGG | 971 | MGDKGTRVFKKASPNGKLTVYLGK |
| | | AAAGCTCACCGTCTACCTGGGAAAGCGGGACTTTGTGGACCACATCG | | RDFVDHIDLVDPVDGVVLVDPEYL |
| | | ACCTCGTGGACCCTGTGGATGGTGTGGTCCTGGTGGATCCTGAGTAT | | KERRVYVTLTCAFRYGREDLDVLG |
| | | CTCAAAGAGCGGAGAGTCTATGTGACGCTGACCTGCGCCTTCCGCTA | | LTFRKDLFVANVQSFPPAPEDKKP |
| | | TGGCCGGGAGGACCTGGATGTCCTGGGCCTGACCTTTCGCAAGGACC | | LTRLQERLIKKLGEHAYPFTFEIP |
| | | TGTTTGTGGCCAACGTACAGTCGTTCCCACCGGCCCCCGAGGACAAG | | PNLPCSVTLQPGPEDTGKACGVDY |
| | | AAGCCCCTGACGCGGCTGCAGGAACGCCTCATCAAGAAGCTGGGCGA | | EVKAFCAENLEEKIHKRNSVRLVI |
| | | GCACGCTTACCCTTTCACCTTTGAGATCCCTCCAAACCTTCCATGTT | | RKVQYAPERPGPQPTAETT |
| | | CTGTGACACTGCAGCCGGGGCCCGAAGACACGGGGAAGGCTTGCGGT | | |
| | | GTGGACTATGAAGTCAAAGCCTTCTGCGCGGAGAATTTGGAGCAGAA | | |
| | | GATCCACAAGCGGAATTCTGTGCGTCTGGTCATCCGGAAGGTTCAGT | | |
| | | ATGCCCCAGAGAGGCCTGGCCCCCAGCCCACAGCCGAGACCACCAG | | |
| prey78687 | 972 | TGTATNGNCATGGGTGACTTTGAGAATGCTGTGCAGTGCCNTGAGCA | 973 | CXXMGDFENAVQCXEQHLKIAXDL |
| | | GCATCTGAAGATAGCCANGGACCTGGGGAACAAGCGAGAAGAGGCCC | | GNKREEARXYSXLGSAYHYRRNFD |
| | | GNNCTTATAGCANCCTGGCCAGTGCCTATCACTACCGGAGGAACTTT | | KANXYHXYVLELXQELMEKAIEMR |
| | | GACAAGGCCATGTNTTACCATANCTATGTCCTGGAGCTGGNACAGGA | | AYAGLGHAARCMQDLERAXQYHEQ |
| | | GTTGATGGAGAAGGCTATTGAGATGCGGGCCTATGCTGGACTAGGCC | | QXGIAEIDLKDRAAEGXASSNLXI |
| | | ATGCTGCCAGGTGCATGCAGGATTTGGAGAGAGCTAANCAATACCAT | | IHQMKGDYDXXL |
| | | GAGCAGCAGNTGGGCATTGCTGAGGATCTCAAGGACCGGGCTGCAGA | | |
| | | GGGGCNAGCATCCTCCAATCTANGAATCATACACCAGATGAAAGGNG | | |
| | | ATTATGACNCTGNNCTG | | |
| prey78693 | 974 | CNNNNNNNNNNNNNNNNNNNNNNNNNNNNNNNNNNNNNNNNNNNNNN | 975 | XXXXXXXXXXXXXXXXXXXXXXXX |
| | | NNNNNNNNNNNNNNNNNNNNNNNNNNNNNNNNNNNNNNNNNNNNNNN | | XXXXXXXXXXX*NYGHALPGLANF |
| | | NNNNNNNNNNGTAGAACTACGGGCATGCACTACCAGGCCTGCCTAAT | | LXXXXXXXXXXXXXXXXXXXXXXX |
| | | TTTTTAATNNNNNNNNNNNNNNNNNNNNNNNNNNNNNNNNNNNNNNN | | XXXXXXXXXXXXXXXXXXXXXXVE |
| | | NNNNNNNNNNNNNNNNNNNNNNNNNNNNNNNNNNNNNNNNNNNNNNN | | KQHSWHIRTTQVNKTGDQINKGKP |
| | | NNNNNNNNNNNNNNNNNNNNNNNNNNNNNNNNNNNNNNNNNNNNNNA | | FCDTVPQAT |
| | | GTGGAGAAGCAGCACTCTTGGCACATAAGGACAACACAAGTCAACAA | | |
| | | GACTGGGGACCAAATTAACAAAGGGAAGCCTTTCTGTGATACAGTCC | | |
| | | CCCAAGCCACC | | |
| prey78702 | 976 | TCNGCANTGATTTTGACTTCCATTTGTGGACTTCATCATCAAATCCT | 977 | SAXTLTCICGLHHQIL*IFVRYRG |
| | | ATGAATTTTTGTTAGATATAGAGGCAGAATATACTGAATTGCCATTC | | RIY*IAILHSNLVTWQW*SFIAVF |
| | | TACATAGCAATTTAGTGACTTGGCAGTGGTAAAGTTTTATTGCAGTT | | CAAE*D*NFSE**CPQLLSQILNS |
| | | TTTTGCGCTGCGGAATGAGATTGAAATTTTTCTGAATAATAATGCCC | | FESWLFL*IS*YFVTNSPKITRQN |
| | | TCAACTATTAAGTCAAATACTTAATAGCTTTGAAAGTTGGCTTTTCC | | SDYKSTLYCGKVTSTRIMNHENQE |
| | | TGTAAATTTCATAATATTTTGTAACAAATTCACCTAAAATTACAAGG | | KEQIVGKCLPIPFPSPS |
| | | CAGAACAGTGATTATAAGTCAACCTTATACTGTGGTAAGGTCACTTC | | |
| | | TACAAGAATAATGAATCATGAAAATCAAGAGAAAGAACAAATAGTAG | | |
| | | GAAAATGTCTTCCAATTCCTTTTAGCCCTTCA | | |
| prey78705 | 978 | GGGGCAAGCTACGTTACATTATGGCAGAAAAAAAGTGCAACTGACAT | 979 | GQATLHYGRKKVQLTYYKEIA*V* |
| | | ACTACAAAGAGATTTTTTAAGTTTAAAAAAAGTTTGGATCTTTTGGA | | KKFGSFGFFFFLGXKGLKNP*III |
| | | TTTTTTTTTTTTTTGGGCNTTAAGGGCCTAAAAAACCCCTAAATTAT | | TPXNKEGFFFFFGPXPKNPVGVFS |
| | | AATAACCCCCNCAAATAAGGAGGGTTTTTTTTTTTTTTTTGGNCCAA | | LSPPSRXFRVPIPKEPPLIYPPGG |
| | | NCCCAAAAAACCCGGTGGGGGTTTTTTCCCTTTCCCCCCCTTCCCGA | | PXVKPPKKNRAPRGNRCNLALLVW |
| | | NTTTTCCGGGTNCCCATCCCCAAGGAACCCCCCCTAATTTACCCCCC | | KNNPPKKTPFFQKXFLX |
| | | AGGGGGGCCTTTNGTAAAACCCCCAAAAAAAAACCGGGCCCCAAGGG | | |
| | | GGAACAGGGGAAACCTGGCCCTTTTGGTTTGGAAAAACAACCCCCCA | | |
| | | AAAAAAACCCCATTTTTTCAAAAANTTTTTTTGCNNAT | | |
| prey78706 | 980 | TCCCATCCTGGTCAATGTTGTGAATGGACTTTACTGGGCACATTCTG | 981 | SHPGQCCEWTLLGTF*DPAGK*YI |
| | | AGATCCTGCCGGAAAGTAATATATATACAGTTGGCCCTCTGTATCTA | | YSWPSVSMDSTNLESKVFRKTIKM |
| | | TGGATTCAACCAATCTTGAATCAAAAGTATTTAGGAAAACAATTAAA | | I*SILGGGA*AICKYHIILYKGLE |
| | | ATGATTTAAAGTATATTGGGAGGAGGTGCATAGGCCATATGCAAATA | | HRWISVSKGSPGNNPLQILRDDCI |
| | | CCACATCATTTTATATAAGGGACTTGAGCATCGCTGGATTTCTGTAT | | QKMNHNDSSCFAGPRIALGHRKFN |
| | | CCAAGGGGAGTCCTGGAAACAATCCCCTGCAGATACTGAGGGACGAC | | FFG*EQSTVED |
| | | TGCATACAAAAAATGAACCATAATGATTCTTCTTGTTTTGCTGGGCC | | |
| | | CAGAATTGCTCTTGCTCATAGAAAATTTAACTTCTTTGGCTAAGAAC | | |
| | | AATCCACGGTAGAAGATGG | | |
| prey78709 | 982 | GGGGAATGTGCAGGGCGAGGAGGCCGTAGGGAAGGCGGGGAGTGGTC | 983 | GNVQGEEAVGKAGSGPSTTGLGAE |
| | | CTAGCACCACAGGGTTGGGAGCAGAGGGAGCGGAGGGCGTTTGCCCC | | GAEGVCPCPGHLCPHSLWGPPAVP |
| | | TGCCCCGGGCATCTCTGCCCTCATTCGCTCTGGGGCCCCCCGGCAGT | | LRRPGPVQSQISTQCWVFLLWL*T |
| | | GCCACTAAGGAGGCCAGGCCCTGTCCAGAGTCAAATCTCCACTCAGT | | GCFLSKHLVYSLRKAATGAN |
| | | GTTGGGTGTTTCTCCTCTGGCTGTAAACAGGGTGTTTCTTAAGTAAA | | |
| | | CATTTAGTATACTCCCTCAGAAAGGCAGCCACAGGGGCAAAC | | |
| prey78711 | 984 | GGCCCCCGCCCAGGCCCCTGTCTCCCAGCCTGATGCCCCTGGCCACC | 985 | APAQAPVSQPDAPGHQRKVVSWID |
| | | AGAGGAAAGTGGTGTCATGGATAGATGTGTATACTCGCGCTACCTGC | | VYTRATCQPREVVVPLTVELMGTV |
| | | CAGCCCCGGGAGGTGGTGGTGCCCTTGACTGTGGAGCTCATGGGCAC | | AKQLVPSCVTVQRCGGCCPDDGLE |
| | | CCTGGCCAAACAGCTGGTGCCCAGCTGCGTGACTGTGCAGCGCTGTG | | CVPTGQHQVRMQILMIRYPSSQLG |
| | | GTGGCTGCTGCCCTGACGATGGCCTGGAGTGTGTGCCCACTGGGCAG | | EMSLEEHSQCECRPKKKDSAVKPD |
| | | CACCAAGTCCGGATGCAGATCCTCATGATCCGGTACCCGAGCAGTCA | | RAATPH |
| | | GCTGGGGGAGATGTCCCTGGAAGAACACAGCCAGTGTGAATGCAGAC | | |
| | | CTAAAAAAAAGGACAGTGCTGTGAAGCCAGACAGGGCTGCCACTCCC | | |
| | | CAC | | |
| prey78718 | 986 | CCTCACTCTGTTGCCCAGGCTGAAGTGCGGTGGCATGATTATGGCTC | 987 | PHSVAQAEVRWHDYGSLAPRHPGL |
| | | ACTATAGCCTCGACATCCTGGACTATGCAATCCTCCCACCTTAGCCT | | CNPPTLAS*VRKTTGMHYQAWLIF |
| | | CCTGAGTACGTAAAACTACGGGCATGCACTACCAGGCCTGGCTAATT | | *XXXXXXXXXXXXXXXXXXXXXXX |
| | | TTTTAATNNNNNNNNNNNNNNNNNNNNNNNNNNNNNNNNNNNNNNNN | | XXXXXXXXXXXXXXXXXXXXXXAR |
| | | NNNNNNNNNNNNNNNNNNNNNNNNNNNNNNNNNNNNNNNNNNNNNNN | | XSTLGT*GQHKSTRXGTKLTKGSL |
| | | NNNNNNNNNNNNNNNNNNNNNNNNNNNNNNNNNNNNNNNNNNNNNAG | | SXIQSPX |
| | | TGAAGAANCAGCACTCTTGGCACATAAGGACAACACAAGTCAACAAG | | |
| | | ACNGGGGACCAAATTAACAAAGGGAAGCCTTTCTNTGATACAGTCCC | | |
| | | CCNAGCC | | |
| prey78486 | 988 | CTCCAGTGCTATGGGAGCATCGGCTACTGCTGGTGTGTCTTCCCCAA | 989 | LQCYGSIGYCWCVFPNGTEVPNTK |
| | | CGGCACGGAGGTCCCCAACACCAGAAGCCGCGGGCACCATAACTGCA | | SRGHHNCSESLELEDPSSGLGVTK |
| | | GTGAGTCACTGGAACTGGAGGACCCGTCTTCTGGGCTGGGTGTGACC | | QDLGPVPM* |
| | | AAGCAGGATCTGGGCCCAGTCCCCATGTGA | | |
| prey78723 | 990 | TTGTCTTCATCCCATGTCATTTTATACAGNACTTTGTAGGTTTTGAT | 991 | LSSCHVILYXTL*VLMRKLVFLMH |
| | | GAGAAAGTTGGTATTTTTAATGCACTCAAAAATTGCTTTTTATTGGA | | SKIAFYWRVLIXITF*SVN*DKRH |
| | | GAGTACTTATCANTATAACTTTCTGATCAGTTAATTAAGATAAAAGG | | TLSTGERIKSVXIIFFCL*KEKLI |
| | | CATACATTGTCCACAGGTGAGAGGATTAAATCAGTTANCATTATTTT | | KFLNGVIKQSFTLIAQAGVQIYCG |
| | | TTTCTGTTTGTAAAAGGAAAAACTTATTAAATTTTTAAACGGAGTTA | | KYKIC*KIHLRN*VIREXMVSGEF |
| | | TTAAACAGAGTTTCACTCTTATTGCCCAGGCTGGAGTGCAAATATAT | | *I*FSMI*HNIH |
| | | TGTGGCAAATATAAAATTTGCTAGAAAATACATCTTAGAAATTAAGT | | |
| | | TATAAGAGAGCANATGGTAAGTGGAGAATTTTAAATTTAATTTTCTA | | |
| | | TGATATAGCATAACATACATTN | | |
| prey78724 | 992 | CGGCGGGGATCCGCAGACCGTGTGGTATGAGTACGGCTGTGTGTGA | 993 | GGDPQTVWYEYGCV* |
| prey84247 | 994 | TAGGTATTCAAGGAGCTGGTCCATTTCANCTNAGTTACTGAATTTAT | 995 | *VFKELVHFXXVTEFMDLDLFIVL |
| | | GGACTTAGACTTGTTTATAGTATTACTTGGTACTTTTAACCTCTNTN | | LGTFNLXXVSNDXPSFIXKKSXXV |
| | | GGGTCTCTAATGATNTTCCCTCTTTCATTATNAAAAAATCANAANTC | | KCLCCI*VYKHHFTTGTPSLXASH |
| | | GTGAAATGTTTGTGCTGTATTTAAGTTTATAAACATCATTTTACCAC | | LPLRXVFXSSITVINNIVRFLEHE |
| | | CGGCACGCCNAGTTTAANTGCTTCCCACCTTCCTTTAAGANAAGTNT | | XTHEGLLLFXXYYXSWFTHPVFGW |
| | | TTNAAAGTAGTATCACTGTCATCAATAACATAGTTCGTTTTCTGGAA | | GKGHNPFFKNQXELXKKPXQLLXX |
| | | CATGAATNAACACATGAGGGTCTGCTGCTATTTATNANATATTATCA | | |
| | | NAGTTGGTTTACACACCCTGTGTTCGGGTGGGGTAAAGGNCATAACC | | |
| | | CTTTTTTTAAAAACCAGCANGAACTTNCTAAAAAACCTNCCCAATTA | | |
| | | CTTTNGNCN | | |
| prey84249 | 996 | GCGAGTGGTTGTTGCGGGGAACGCTGGTGCGGGCGGCCGTGCGGCGC | 997 | EWLLRGTLVRAAVRRYLPWALVAS |
| | | TACCTGCCCTGGGCCCTGGTGGCCTCCATGCTGGCGGGCTCCCTCCT | | MLAGSLLKELSPLPESYLSNKRNV |
| | | CAAGGAGTTGTCCCCGTTGCCCGAGAGCTACCTCAGCAACAAGCGCA | | LNVYFVKVAWAWTFCLLLPFIALT |
| | | ACGTCCTCAACGTGTATTTTGTCAAAGTGGCCTGGGCCTGGACGTTC | | NYHLTGKAGLVLRRLSTLLVGTAI |
| | | TGTCTCCTTCTGCCTTTCATTGCCCTCACCAACTACCATCTGACCGG | | WYICTSIFSNIEHYTGSCYQSFAL |
| | | CAAGGCTGGCTTGGTCCTGCGGCGCCTGAGCACCCTGCTTGTGGGCA | | EGVRKEHQSKQQCHQEGGFWHGFD |
| | | CGGCCATCTGGTACATCTGCACCTCCATCTTCTCCAACATCGAACAC | | ISGHSFLLTFCALMIVEEMSVLHE |
| | | TACACGGGCAGCTGCTACCAGTCCCCAGCCCTGGAGGGGGTCAGAAA | | VKTDRSHCLHTAITTLVVALGILT |
| | | GGAACACCAGAGCAAGCACCAGTGCCACCAGGAAGGGGGCTTTTGGC | | FIWVLMFLCTAVYFHNLSQKVFGT |
| | | ATGGCTTTGACATCTCAGGTCACTCCTTCCTGCTGACCTTCTGCGCC | | LFGLLSWYGTYGFWYPKAFSPGLP |
| | | CTCATGATTGTAGAAGAGATGTCTGTGCTGCATGAGGTGAAGACGGA | | P |
| | | CCGAAGCCACTGCCTCCACACCGCCATCACCACCCTGGTTGTGGCCC | | |
| | | TGGGCATTCTGACTTTCATCTGGGTGTTGATGTTTCTGTGCACAGCT | | |
| | | CTTTATTTCCACAACTTGTCCCAGAAGGTGTTTGGCACCTTGTTTGG | | |
| | | TTTGCTGAGCTGGTACGGGACATACGGGTTTTGGTATCCGAAAGCCT | | |
| | | TTTCCCCAGACTTCCTCCC | | |
| prey84253 | 998 | GGGANNNNNNNNNNNNNNNNNNNNNNNNNNNNNNNNNNNNNNNNNNN | 999 | GXXXXXXXXXXXXXXXXXXXXXXX |
| | | NNNNNNNNNNNNNNNNNNNNNNNNNNNNNNNNNNNNNNNNNNNNNNN | | XXXXXXXXXXXXXXXXXVPLYFAN |
| | | NNNNNNNNNNNNNNNNNNNNNNNNNNNATGTACCACTGTACCCAGCT | | LXXXXXXXXXXXXXXXXXXXXXXX |
| | | AATCTTTTNNNNNNNNNNNNNNNNNNNNNNNNNNNNNNNNNNNNNNN | | XXXXXXXXXXXXAHTARLIFDLRW |
| | | NNNNNNNNNNNNNNNNNNNNNNNNNNNNNNNNNNNNNNNNNNNNNNN | | VFGGRNPAVRSICTHIPS*WWTCL |
| | | NNNNNNNNNNNNNNNNNGCACACACAGCCAGATTGATTTTTGATTTA | | YS*FC*VLLSVLSGSI |
| | | CGATGGGTTTTTGGGGGGCGGAACCCTGCTGTCAGGAGCATCTGTAC | | |
| | | TCATATTCCCTCATGATGGTGGACTTGTTTATATTCGTGATTCTGTT | | |
| | | GAGTTTTGCTTTCTGTGCTTTCAGGCTCTATC | | |
| prey70344 | 1000 | AGTTCAACATAGAGAAAGGTCGGCTTGTGCAAACCCAAAGACTAAAG | 1001 | FNIEKGRLVQTQRLKIMEYYEKKE |
| | | ATTATGGAATATTATGAGAAGAAAGAGAAACAGATTGAGCAGCAGAA | | KQIEQQKKIQMSNLMNQARLKVLR |
| | | GAAAATTCAGATGTCCAATTTGATGAATCAAGCGAGACTCAAAGTCC | | ARDDLITDLLNEAKQRLSKVVKDT |
| | | TCACAGCAAGAGATGACCTTATCACAGACCTACTAAATGAAGCAAAA | | TRYQVLLDGLVLQGLYQLLEPRMI |
| | | CAGAGACTCAGCAAGGTGGTAAAAGATACAACCAGGTACCAAGTGCT | | VRCRKQDFPLVKAAVQKAIPMYKI |
| | | GCTGGATGGACTGGTTCTCCAGGGTTTGTACCAGTTGCTGGAGCCCC | | ATKNDVDVQIDQESYLPEDIAGGV |
| | | GAATGATTGTTCGTTGCAGGAAACAAGATTTCCCTCTGGTAAAGGCT | | EIYNGDRKIKVSNTLESRLDLIAQ |
| | | GCAGTGCAGAAGGCAATTCCTATGTACAAAATTGCCACCAAAAACGA | | QMMPEVRGALFGANANRKFLD* |
| | | TGTTGATGTCCAAATTGACCAGGAGTCCTACCTGCCTGAAGACATAG | | |
| | | CTGGTGGAGTTGAGATCTATAATGGAGATCGTAAAATAAAGGTTTCC | | |
| | | AACACCCTGGAAAGCCGGCTGGATCTCATAGCCCAGCAGATGATGCC | | |
| | | AGAAGTCCGGGGAGCCTTGTTTGGTGCAAATGCCAACAGGAAGTTTT | | |
| | | TGGACTAA | | |
| prey84262 | 1002 | TCTTCCACCAAGACAGAGACGTTAGCAACGCATGGCGGGTGGGGACC | 1103 | SSTKTETLATHGGWGPGVLRRGYP |
| | | TGGGGTGCTCAGGAGGGGGTACCCGGGGCCCCGGCCAGAGATACATC | | GPRPEIHQLHPRGGTADGSQHQQD |
| | | AATTACACCCCCGTGGGGGGACAGCCGATGGGAGCCAGCACCAGCAG | | PRAPRTEVCPTHFLPTTCAPESRA |
| | | GATCCGAGGGCGCCCCGGACAGAGGTCTGCCCCACCCACTTCCTCCC | | CPGRWRPGVECTCSHEVLGVF*ES |
| | | CACCACCTGTGCCCCAGAGAGCAGGGCCTGCCCGGGAAGGTGGCGTC | | LSDTTLV*PHRVVSNIHGSGYGMV |
| | | CTGGAGTCGAGTGTACCTGCAGCCATGAGGTTCTGGGTGTTTTTTGA | | YLCNLGCADG*LVS |
| | | GAGAGTCTGAGTGACACCACACTCGTGTGACCCCACAGGGTTGTGTC | | |
| | | CAACATACACGGAAGTGGCTATGGAATGGTGTATTTGTGCAACCTGG | | |
| | | GGTGCGCGGATGCGTGACTTGTATCTAN | | |
| prey84427 | 1004 | NCCACANTGTCCTGCCAAATCCTGCCAAATGTTCCTGGAAGACAAAA | 1005 | XTXSCQILPNVPGRQNIPSTPVYR |
| | | CATACCCTCTACTCCAGTGTATAGAATTACTGTACTAGAATATCAGC | | ITVLEYQLFEID*MVILSLPNC*F |
| | | TTTTTGAAATTGATTAAATGGTTATACTTTCTCTTCCCAACTGCTGA | | VAGILKVWSLTYLGGGSFLKLQVP |
| | | TTTGTAGCTGGAATTTTAAAAGTGTGGTCTTTGACTTACTTGGGAGG | | ELILDLLNQKR*R*NPEI*FLQDT |
| | | TGGTAGCTTCTTAAAATTGCAGGTTCCTGAGCTTATCCTAGACCTAC | | TFPPPTR*FCPRLDPTVAALLHYL |
| | | TGAATCAGAAACGCTGAAGGTAGAACCCAGAGATCTGATTTTTACAA | | HXFSPASLTGVPALLPLXAPMMPN |
| | | GACACTACCTTCCCACCCCCAACCAGGTAATTCTGCCCTAGGCTTGA | | |
| | | CCCTACTGTGGCATAACTGCTGCATTATCTACATTTNTTTTCTCCTG | | |
| | | CAAGTCTTACTGGTGTTCCAGCTTTGCTTCCNCTTTTNGCACCAATG | | |
| | | ATGCCAAACA | | |
| prey84267 | 1006 | ANNATGTGCCTGTANTNAAACNGACCNCNAGCNNGCTGCATGCTNCC | 1007 | XMCLXXNXPXIIXCMLPLXKXXXX |
| | | ACTCNCNAAGNGGNGNNACAGNCNGGACTGGTGGCCTTNGACTTGTC | | XDWWPXTCLCCXLFXPXXXFSXXA |
| | | TNTGTTGCTNATTGTTCNTGCCTTGNNNTTNCTTTAGCNNTNCTGCA | | PXPXTXGAVXXEW*XRTLXSXXXX |
| | | CCGANCCCNTNGACNANTGGNGCNGTGNCCTNGGAGTGGTGANNNAG | | ALTAYVNGXXXXMRXRXMXXX*XR |
| | | AACACTTNGTTCNNTTTNN1AGCCNCTGACTGACCGCATATGTTAAT | | GXPSYVXXRGXNAPGXXG*LGFXX |
| | | GGGNTGGNTNTCNGTATGAGACANAGANCTATGNGGNTTNGCTGATN | | WACVXXTXGXPWXPXGFXAGFRXP |
| | | AAGAGGNNGCCCNAGCTACGTTCNTTNTCGACGTNCTAATGCGCCTC | | XXPN |
| | | GCCANTANGGATGATTGGGGTTTNTTGNCTGGGCTTGCGTTTTNCNA | | |
| | | ACTTANGGGNCCCCCTGGNGCCCCCNTGGGTTTTNNTAGGGNTTTAG | | |
| | | ANGGCCTTNGGNCCCCAATT | | |
| prey84271 | 1008 | GGGANNNNNNNNNNNNNNNNNNNNNNNNNNNNNNNNNNNNNNNNNNN | 1009 | GXXXXXXXXXXXXXXXXXXXXXXX |
| | | NNNNNNNNNNNNNNNNNNNNNNNNNNNNNNNNNNNNNNNNNNNNNNN | | XXXXXXXXXXXXXXXXXVPLYPAN |
| | | NNNNNNNNNNNNNNNNNNNNNNNNNNNATGTACCACTGTACCCAGCT | | LXXXXXXXXXXXXXXXXXXXXXXX |
| | | AATCTTTTNNNNNNNNNNNNNNNNNNNNNNNNNNNNNNNNNNNNNNN | | XXXXXXXXXXXXAHTARLIFDLRW |
| | | NNNNNNNNNNNNNNNNNNNNNNNNNNNNNNNNNNNNNNNNNNNNNNN | | VFGGRNPAVRSICTHIPS*WWTCL |
| | | NNNNNNNNNNNNNNNNNGCACACACAGCCAGATTGATTTTTGATTTA | | YS*FC* |
| | | CGATGGGTTTTTGGGGGGCGGAACCCTGCTGTCAGGAGCATCTGTAC | | |
| | | TCATATTCCCTCATGATGGTGGACTTGTTTATATTCGTGATTCTGTT | | |
| | | GAGT | | |
| prey84276 | 1010 | ACATGAAAGGACTCACACTGGAGAGAAACCCTATGGTTGTCAGCAAT | 1011 | HERTHTGEKPYGCQQCGKALSDLS |
| | | GTGGGAAAGCATTATCTGATCTCTCAAGCTTTCGAAGACACATGATA | | SFRRHDATTHTGNGPHKCKICGKG |
| | | ACACATACTGGAAATGGACCTCATAAATGTAAGATATGTGGGAAAGG | | FDYPSSVQRHERTHTGEKPYECKE |
| | | CTTTGATTATCCCAGTTCAGTGCAAAGACATGAAACAACTCACACTG | | CGKAFSHSSYLRIHERVHTGEKPY |
| | | GAGAGAAACCCTATGAATGCAAGGAATCTGGTAAAGCCTTCAGTCAT | | KCKECGKPFHCPSAFHKHERTHSM |
| | | TTACGAATACACGAAAGAGTTCATACTGGAGAGAAGCCGTATAAATG | | EKPYKCKECGEAFHCISSFHKHEM |
| | | TAAGGAATGTGGGATCCCAGTGCCTTTCATAAACATGAAAGGACCCA | | TH* |
| | | CAGTATGGAGAAACCGAATGTGGGGAAGCATTTCATTGTATCAGTTC | | |
| | | CTTTCATAAACATGAA | | |
| prey84278 | 1012 | TCTTATTTGNNNNNNNNNNNNNNNNNNNNNNNNNNNNNNNNNNNNNN | 1013 | SYLXXXXXXXXXXXXXXXXXXXXX |
| | | NNNNNNNNNNNNNNNNNNNNNNNNNNNNNNNNNNNNNNNNNNNNNNN | | XXXXXXXXXXXXXXXXXXXXXXXX |
| | | NNNNNNNNNNNNNNNNNNNNNCCAGCTGATTTTTAAGTTTTTANTTT | | XXXXXXPADF*VFFFXKQXXXXXP |
| | | NNNTTNGNCCCCNNGGNNGNNNCNCNNNGNNNCAATTTAANGCNNGC | | XXXXXXXI*XNXTLXXXG*XIFXX |
| | | ANGGTTAAANNATTTTTCNNNCNTAACCNTCCNNAGNNNNNCCCNNC | | *PSXXXGXXGXPXHXGXILKFLEK |
| | | CACNTTGGNTNNATTTTAAAGTTTTTGGAAAAAAGGGGNAAGGGGGG | | RGFPXXAKGGXXPXGXKXS |
| | | TNTNNACCCCTNGGGCNTCAAGNANTCCTC | | |
| prey84280 | 1014 | TACTTCCTTTGAAATAGTAAAATTCTATTTTATTTATTTATTTATTT | 1015 | YFL*NSKILFYLFIYLFETESRXX |
| | | ATTTGAGACGGAGTNNNNNNNNNNNNNNNNNNNNNNNNNNNNNNNNG | | XXXXXXXXXXXXGKKKXXXXXXXG |
| | | GAAAAAAAAAANNNCNGGGCANCCCNCCNTTNAAAAAAAAAAAANNN | | XPPXKKKKXETXXGKXXXXGXGTX |
| | | GAAACCNTNNGGNTNGGANNGGGNACCNGACNTCAGNNTTNTTNACC | | XQXXXPGGKXXPDXKKIXPLSXXS |
| | | GGGGGGGAAAAAAAAAATTTTNCCCCTTTCCCNCNGGAGTNTNTAAA | | X*KXXXGAXGGLXGXVFSXXKKKX |
| | | AANAAAANANCCTNNGGGGGNTNGTTTTTTCAAANGNTAAAAAAAAA | | PFFFXKXGXXXFFXXRKKKXFFFF |
| | | ANNCCCTTTGGATNTNCTNGTTTTTTTNNGANCCGAAAAAAAAAGNT | | |
| | | TTTTTTTT | | |
| prey84285 | 1016 | CCGTCCCCTACACGGATGAGGCCCGGCGGTCGCGCCTCAACCGGCTG | 1017 | MPWLAVPYTDEARRSRLNRLYGIQ |
| | | TATCCCCACGCTCATCATGCTGGACCCGCAGGGCGAGGTGATCACGC | | GIPTLIMLDPQGEVITRQGRVEVL |
| | | GAGGTGCTGAACGACGAGGACTGCCGGGAGTTCCCCTGGCACCCCAA | | NDEDCREFPWHPKPVLELSDSNAA |
| | | GCTCCGACTCCAACGCCGCGCAGCTTAACGAGGGCCCCTGCCTCGTC | | QLNEGPCLVLFVDSEDDGESEAAK |
| | | CGAGGATGACGGAGAGTCCGAGGCGGCCAAGCAGCTGATTCAGCCGA | | QLIQPIAEKIIAKYKAKEEEAPLL |
| | | TTTGCCAAGTACAAAGCCAAAGAGGAGGAGGCACCCCTTCTGTTCTT | | FFVAGEDDMTDSLRDYTNLPEAAP |
| | | CTGACATGACTGACTCCCTGCGAGATTACACCAACCTGCCTGAGGCT | | LLTILDMSARAKYVMDVEEITPAI |
| | | GATCCTGGACATGTCAGCCCGGGCCAAGTACGTGATGGACGTGGAGG | | VEAFVNDFLAEKLKPEPI* |
| | | ATCGTGGAGGCCTTTGTGAATGACTTCCTAGCAGAGAAGCTCAAACC | | |
| | | G | | |
| prey84298 | 1018 | AGCTGTCCTGAATAAATGTCACAATCAGACTTGGAAGAAACACTGTC | 1019 | LCFL*LS*INVTIRLGRNTVLGKE |
| | | CTTCCTACTAAGGATAGGCAGTTTAAGGCTCAAGCCCTGAGCTATTT | | *FLLRIGSLRLKP*AISAEPPWDQ |
| | | CGGGATCAGAGAGGAAAATTCAGCACCACAGAGACCTGGCTCAGCTC | | RGKFSTTETWLSSAGSWVLTMVPT |
| | | ACCTCACTATGGTCCCTACCTGGCCCCCACCTGTTGGAGTGGCTCAG | | WPPPVGVAQFREKRHILRSLVCAK |
| | | TCACATCCTGCGCTCTCTTGTCTGTGCCAAGTGATCAGAAGCTCAGT | | *SEAQFWGWGCANYFSTSNQVTLT |
| | | TGTGCAAACTATTTTTCTACAAGCAACCAAGTGACTCTAACATGTAG | | CSQC*KPLPQQIHEPLSTN |
| | | CACTGCCCCAACAGATCCACGAACCTCTAAGCACCAT | | |
| prey84299 | 1020 | CCACCACACCCGGCTCAATTTTGTATATNTTGTAGTNNNNNNNNNNN | 1021 | TVARTTTPGSILYXL*XXXXXXXX |
| | | NNNNNNNNNNNNNNNNNGTTTGAGCATTCCTCCCACCTCGGCCCTCC | | XXXXXXXLSIPPTSALXSARDYKQ |
| | | NATAGGCAAGAGCCACTGTGCCCAGCTAAGATNTATTTTAAAACAAC | | EPLCPAKXYFKTTVSRIFVFWTGI |
| | | ATGTATTTTGGACAGGCATCTCTTGTCAGATGTATATATNGTGAATA | | SCQMYIX*ISLPXXWVLILSXEXK |
| | | TTGGGTCCTCATTCTGTCTTTNGAGGNGAAGGAGTTCTATTAAATCT | | EFY*IFFSLLXILSIIF*RXSNNY |
| | | TCAATTTTGTCCATTATATTTTGAAGATNTTCAAATAATTATTGCTT | | CFLLXNMVRYMVLRE*RTSXP*PT |
| | | TGGTTAGGTATATGGTTCTCAGAGAATAAAGGACTTCTNAACCGTAA | | TK |
| | | C | | |
| prey84304 | 1022 | NNNNNNNNNNNNNNNNNNNNNNNNNNNNNNNNNNNNNNNNNNNNNNN | 1023 | XXXXXXXXXXXXXXXXXXXXXXXX |
| | | NTAGGAAAATTTTAAATTGCATATGTAGTTCACATTCTATTTCTCAC | | TYLGKFAIAYVVHILFLLDSTGLE |
| | | TGGTCTAGAAGTGTAGGTTGGCCATGGGCGGAGCCTACCTCCCAGGC | | V*VGHGRSLPPRLPQTFIQVIVIP |
| | | TTTTCATACAAGTGATTGTCATTCCTATCCCCTCAGTTTTTCAATCA | | IPSVFQSFFLYSSQISEW*SVGHL |
| | | TTCTTCCTCAGAGTGGTAGAGTGTAGGCCACTTGCTGAGCTGGGTGT | | LSWVYHLGKPV*NTGTGEPSP*SL |
| | | ATCAGCCTGTTTAAAATACAGGCACTGGAGAACCCTCTCCATAAAGT | | IXKDWTG |
| | | CTGATTTANGGCAC | | |
| prey9012 | 1024 | AACTTCAGAATGCTGGTGGCAAGAGCTGGGCCCCAGAGATAGCTACT | 1025 | SLHKQLQNAGGKSWAPEIATHPSG |
| | | CATAACCAGCAGAAAAGGCTGTCCTGGGACAAGTTGGATCATCTGAA | | LHNQQKRLSWDKLDHLMNEEQQLL |
| | | CCTGCTTTGGCAAGAGAATGAGAGGTCCCAGACCATGGTACAGAACA | | WQENERSQTMVQNTKAELTHSREK |
| | | CGCACTCCCGGGAGAAGGTCCCTCAATTGGAATCCAATCTTCTTCAT | | VRQLESNLLPKHQKHLNPSGTMNP |
| | | CTAAACCCATCAGGTACCATGAATCCCACAGAGCAAGAAAAATAGTG | | TEQEKLSLKRECDQFQKEQSPANR |
| | | TGATCAGTTTCAGAAAGAACAATCTCCTGCTAACAGGAAGGTCCTTG | | KVSQMNSLEQELETIHLENEGLKK |
| | | AACAAGAATTAGAAACAATTCATTTGGAAAATGAAGGCCTGAAACTG | | KQVKLDEQLMEMQHLRSTATPSPS |
| | | GATGAGCAGCTCATGGAGATGCAGCACCTGAGGTCCACTGCCCCTCA | | PHAWDLQLLQQQACPMVPREQFLQ |
| | | TGCTTGGGATTTGCAGCTGCTCCAGCAGCAAGCCTGTCCGCAGTTTC | | LQRQLLQAERINQHLQEELENRTS |
| | | TGCAGCTTCAACGCCAGCTGCTGCAGGCAGAAAGGATAGAGGAACTT | | ETNTPQGNQEQLVTVMEERMIEVE |
| | | GAAAACAGGACCTCCGAAACCAACACACCACAGGGAATAACTGTCAT | | QKLKLVKRLLQEKVNQLKEQLCKN |
| | | GGAGGAACGAATGATAGAAGTTGAACAGAAACTGAATCTTCAAGAGA | | TKADAMVKDLYVENAQVC* |
| | | AAGTGAATCAGCTCAAAGAACAACTCTGCAAGAACTGAAGGACTTGT | | |
| | | ATGTTGAAAATGCCCAGGTTTGTTGA | | |
| prey84310 | 1026 | ACGGGGCAAGCTACGTTACATTATGGCAGAAAAAAAGTGCAACTGAC | 1027 | TGQATLHYGRKKVQLTYYKEIF*V |
| | | ATACTACAAAGAGATTTTTTAAGTTTAAAAAAGTTTGGATCTTTTGG | | *KKFGSFGFLFFFXGFXXQKXXXX |
| | | ATTTCTTTTTTTTTTTTNGGGNTTTANGGNCCAAAAAANCCNTTANT | | XXPPXXXXXXFFFLGPPQKXXXXX |
| | | TAANAATNACCCCCCCNAANNNGGGNGGNTTTNTTTTTTTTTTTGGG | | FSXXXXSXFXXXXXPXXXSXXXPR |
| | | CCCNCCNCAAAAANCNNGGNANNGGGNNTTTTCCNTNNNCCNCNTTT | | XGXXWXNXXXKTTAPXGGXRGXXX |
| | | CCNGTTTTTCNANGGNGGNNTCCNTTNNGTTNCCCCGGNGGGGNNNT | | XPFXXXXKXXAPXXKNPXFFGXXX |
| | | NNTTGGGNAACAACGGCGCCCNANGGGGGGGNCCGNGGGAANNNCNT | | |
| | | NNCCCCCAAANCGNGGGCCCCNCNCANAAAAAACCCCCNTTTTTTTG | | |
| | | GNCANA | | |
| prey84412 | 1028 | CATTATGGCAGAAAAAAAGTGCAACTGACATACTACAAAGAGATTTA | 1029 | GQATLHYGRKKVQLTYYKETF*V* |
| | | GTTTGGATCTTTTGGATTTCTTTTTTTTTTTTNGGGNTTTANGGNAT | | KKFGSFGFLFFFXGFXXXKNP*LK |
| | | TAAAAATACCCCCCCNAATTAGGGNGGTTTTTTTTTTTTTTTTGANC | | IPPXIRXVFFFFLGXXXKXRXCVF |
| | | CGGGNNGGGGTTTTTNCCNTTNCCCCCCNTTCCGGATTTTGNNAGGA | | XXXPXSGFXXXPXPKXPPLXXPQG |
| | | NCCCCCCTTANTTNNCCCCCAAGGGGGGCCNTTTGGTGAACCCGGNC | | GPFGEPXKKXPGXKGGTGEPGPFL |
| | | NCAAGGGGGGNACGGGGGAACCCGGCCCCTTTTTGNTTTNGNAAAAA | | XXEKKGPXKKPHFFKKXFFSXXGA |
| | | AAACCCCATTTTTTTAAAAAAANTTTTTTTTCTATNNAGGANTCCCC | | PXXSPXX |
| | | ANAAAAN | | |
| prey84314 | 1030 | NNNNNNNNNNNNNNNNNNNNNNNNNNNNNNNNNNNNNNNNNNNNNNN | 1031 | XXXXXXXXXXXXXXXXXXXXXXXX |
| | | NNNNNNNNNNNNNNNNNNNNNNNNNNNNNNNNNNNNNNNNNNNNNNN | | XXXXXXXXXXXXXXXXXXXXXXXX |
| | | NNNNNNNNNNNNNNNNNNNNNNNNNNNTTTTTGGAAAAANGNNGGGN | | XXXXXXXFLEKXXFXXXXXXGXNX |
| | | GGGNTTNAACNNCNGGGNNTAANGGNNTTNGCCCTTNTAACGGGANT | | XX*XXXPX*PXKGXGXXGXXXXPX |
| | | ANCGGGNTNNANNNTTNGCCCCNGNCCGGGCCCGGGGNNNAAAATNG | | PGPGXKXLXXNXXX*KXFXXGXXG |
| | | GANNNTTTAAAAATNTTTTNGGTNNGGGNCCCNGGGGCCCCNTTAAA | | PXXXXXVKXPGPX |
| | | NGNCCCGGNCCCNATTT | | |
| prey84315 | 1032 | TCAGACCNGNTGNATTAAATATTNCNNCTTNNGNTNTNNANAAAGGN | 1033 | XLXSFRPXXLNIXXXXXXKGXXXL |
| | | NTATTCCACCTGTCATCCTAATTTATGCACAAANTATTCAAATATCT | | FHLSS*FMHKXFKYLTHLTSLLPA |
| | | TACTCAATTGCTTCCAGCCGTTCAAACCAGGGCCAGATGAGGTAATC | | VQTRAR*GNQS*R*NCHQRRSSSY |
| | | AATCATAGAGACACCAAAGAAGGTCGTCTTCTTATTAGTCAGAACCT | | *SEPEAH*TA*KYFLLVHVVRLTR |
| | | GAACATTAAACAGCATTCTTACTTGTGCATGTAGTAAGACTCACTAG | | NAEGK*VEQVREAVFTPMYPSWVF |
| | | AAATGCTGAAGGGAAATAAGTGAGAGAAGCAGTTTTTACTCCGATGT | | F*SKSLIKL*NLVVWDEVNNQQXR |
| | | ATCCAAGTTGGGTTTTTTTCTGATCAATTAAGCTTTAAAATCTGGTG | | |
| | | GTCTGGGATGAAGTAAACAATCAACAACNTA | | |
| prey84321 | 1034 | ACGAGGTTTTCACTATGTTGCCCAAGCTGAAATTTCTAGTTTTGATG | 1035 | YFL*RRGFHYVAQAEISSFDDYIL |
| | | TTATTTAGAACAAGTTTCATGTTTTTAAAAAAGCAAAGCAAAACTCA | | HYLEQVSCF*KSKAKLRESPY*AI |
| | | GTGAGCTATTTTGAATAGATTTTTAAGNNNNNNNNNNNNNNNNNNNN | | LNRFLXXXXXXXXXXXXXXXXXXX |
| | | NNNNNNNNNNNNNNNNNNNNNNNNNNNNNNNNNNNNNNNNNNNNNNN | | XXXXXXXXXXXXXXXXXXXHVNHH |
| | | NNNNNNNNNNNNNNNNNNNCATGTAAACCACCATGTGTGCTCTCAAA | | VCSQIDF*MIENH |
| | | TAGAGATTGAGAATCACCT | | |
| prey84323 | 1036 | GGATTTCTTTAAATCTAATGGCAGCTGATCTCANNAATTCTGATGGC | 1037 | GSIILGFL*I*WQLISXILMGSSW |
| | | TACGGAACTCCTATTAAGAGCANAGCNCTNTTTTTATCTNTTTTGCT | | SYGTPIKSXALFLSXLLGLRCVIX |
| | | GTTATTNTNAAAAAATAATGTGCTTTTTTGNGTGGTGATCCTATGCT | | KK*CAFLXGDPMLGTWH*KLSSKY |
| | | TAGAAGTTATCTTCCAAATATACTGGTGGTTTCCTAGAAGTATATAT | | TGGFLEVYIGLNISN*NLSCMNIA |
| | | TGTAACTAGAACTTATCATGTATGAACATTGCATTTTGGATTGAATA | | FWIE*LRYVRISLP**IIFLWKKW |
| | | GCTATATCTCTGCCATGATAAATTATTTTTTTATGGAAGAAATGGTC | | SHFXN*QIFWELIGFKGQFMLYIE |
| | | TCATAAATTTTTTGGGAGTTGATAGGATTTAAAGGACAGTTCATGCT | | SISYLFQVRWEKVTM*VCIVVIYL |
| | | GTATACTATTTGTTTCAAGTCAGGTGGGAGAAAGTAACTATGTAGGT | | NFQGSLVPVLTLSA |
| | | ATGTATTTGAATTTTCAAGGTTCTTTAGTACCGGTCTTAACCTTAAG | | |
| | | CGCGAG | | |
| prey56846 | 1038 | ACACAAAGATGTTTGAGGAGCTCAAGAGCCGTCTGGACACCCTGGCC | 1039 | KDVVNTKMFEELKSRLDTLAQEVA |
| | | TGCTGAAGGAGCAGCAGGCCCTGCAGACGGTCTGCCTGAAGGGGACC | | LLKEQQALQTVCLKGTKVHMKCFL |
| | | AATCCTTTCTGGCCTTCACCCAGACGAAGACCTTCCACGAGGCCAGC | | AFTQTKTFHEASEDCFSRGGTLST |
| | | GACGCGGGGGCACCCTGAGCACCCCTCAGACTGGCTCGGAGAACGAC | | PQTGSENDALYEYLRQSVGNEAEI |
| | | GCCTGCGCCAGAGCGTGGGCAACGAGGCCGAGATCTGGCTGGGCCTC | | WLGLNDMAAEGTWVDMTGARIAYK |
| | | AACGGGGCACCTGGGTGGACATGACCGGCGCCCGCATCGCCTACAAG | | NWETEITAQPDGGKTENCAVLSGA |
| | | AACTGACCGCGCAACCCGATGGCGGCAAGACCGAGAACTGCGCGGTC | | ANGKWFDKRCRDQLPYICQFGIV* |
| | | CTGTCAGCAAGTGGTTCGACAAGCGCTGCCGCGATCAGCTGCCCTAC | | |
| | | ATCTGCCTCGTGTAG | | |
| prey84327 | 1040 | GGGTGGGAACCCGGGTTGTCCCTCTTTCCTGTCCCCACCAGCCCATG | 1041 | EEEVGWEPGLSLFPVPTSPCCLPR |
| | | CTGGACTGTGGCACATGTGGGTGAAGAGCACACTCTGCAGTCAGACT | | TVAHVGEEHTLQSDYGSSSPGSWT |
| | | ACGGGAGCCTGGATCCTGGACACTGCTTGCCGTGATTTAGCTTTCTC | | LLAVI*LSLPKCGRQYW*LPQRLM |
| | | TGCCAAAATGTGGGCTACCACAGAGGCTTATGGTGATATTAGACGPA | | VIIRGNDMCNTRSPVPGK*SCLLN |
| | | ATGACATGTGCACCCAGTGCCTGGCAAATAGTCCTGCCTGCTAATGT | | VSGDDGNDENSC*RLLSRPEVRLL |
| | | CAGTCGTGATGATGGAATTCGTGTTAAAGACTGTTGTCCAGGCCAGA | | XLVIRVR*LYCS*LKKVTHFSNYK |
| | | GGTGAGGCTCCTAGNTCTATGAGATGATTATATTGCTCTTAGCTAAA | | X |
| | | AGTGACACATTTTTCTAATT | | |
| prey84334 | 1042 | GCNTCTGGNATGGNTGTGAANCNTTGAGGCGAANNCGANNACCCNTG | 1043 | XXXXSXWXGCEXLRRXRXPXXAXE |
| | | NAATGCCTACAGTGNNNGNATNAGGAANATGNTGTGCNTCAGANGCC | | MPTVXXXGXXCASXAPXGXXKXXV |
| | | CCCCNAAANNNNCNGTACCTTTCTGAACGAGCANTANCNNGGCGGCC | | PF*TSXXXAAXXXYHXXXXCASWP |
| | | ANNTTNANCNGNCCTNCTGTTAGAGCTGGCCCTNTNNNNACCCNACC | | XXXPT*TXXCXQLHPTDXXXEACP |
| | | TAAAACAGANGNNTTCACCCAACNGATNNTGNGNNCGAAGCCTGTCC | | CSXPNATLVIILLPMVRGCXQTXS |
| | | ATCTTCGGNCCCCAATGCATCCTGCTCCCTATGGTGACAGGATGTTT | | AXXXTLXSWXTLFLLVX |
| | | NCAAACCAGNTCTGCANNGNNCCCTCCTC3GGNCACCCTNTTCCTCC | | |
| | | TAGTGTNC | | |
| prey84337 | 1044 | TACTTANTTTAAGGTCCTTTNGGGTNNCTTNNAGNNNTTTNTGAATN | 1045 | AIAGILXLRSFXVXXXXX*XLNXT |
| | | TNCGTNACCNCCTTANCNTNTTTANTTAAAATTAAANTNGNCGTNAC | | RXXLXXFXLKLXXRXLXTF*KGKP |
| | | CTTTAAAAGGAAAACCTTTTACCCNTTAAAGTTAAANTCCGGNGGGN | | FTX*S*XPXGPPXRGKXLFFSPWG |
| | | CCCCCCCNGNCTTTTTTTTTCCCCTTGGGGGGAANCNTTAAAANTTN | | EXXKXXFXXXGSXXPPKPXKXFXP |
| | | NCTTTTTNNNANNNNCCCCCCAAAACCCCNNAAAANNTTTNCCCCNC | | LXEKRGPXFLKKN |
| | | TTNGNGAAAAAGGGGTTAAAAAAAAATTT | | |
| prey84338 | 1046 | GTTGGTTTTGAGCACATCTTGCCAGTTCTGTGTCTATTTGGGGATCT | 1047 | LRHLGWF*AHLASSVSIWGSFK*N |
| | | TTTCTGATTCATACTCAATATAATTAGGATCTGCAGCCCCAGGCTTC | | SDSYSI*LGSAAPGFMPANGKIHS |
| | | ATGCCAAAATCCATTCATCTAGTTGGAATGAAATGATCAGAAAGGGG | | SSWNEMIRKGKENEWNEIKIL*SH |
| | | AAGGAGAATAAATTAAAATATTATAATCTCACTCCCTGGATGCCACA | | SLDATRECIILESLIS*RAKDSYR |
| | | AGGGAGTGCATCACCTAATTTCCTAGCGAGCTAAGGATTCATACCGA | | ETQI*MSVSPDSNISM*ISPILPS |
| | | GAAACACAAATTTAAATCCTGACAGTAACATAAGCATGTGAATCAGC | | REWWDLRRTGXCLSSLKEFGLX |
| | | CCAATACTGCCCAGCAGGGAGTGCGGAGGACTGGANAGTGCCTGTCT | | |
| | | AGCCTAAAGGAATTCGGATTANAAN | | |
| prey84344 | 1048 | ACATACAGTGACAATCCCCAAACGGTGTTTCCTCGCTACTGTGTTAG | 1049 | DYLLTYSDNPQTVFPRYCVSWMVS |
| | | TCCAGTGGCATGCCAGATTTCCTGGAGAAGCTGCACATGGCCACTCT | | SGMPDFLEKLHMATLKAKNMEIKV |
| | | GAAAGGGAGATTAAAGTAAAGGACTACATCTCAGCTAAGCCTCTGGA | | KDYISAKPLENSSEAKATSQSSERKNEGSCGPARIEYA* |
| | | AATGAGTAGGCCACCAGCCAGTCCTCTGAGCGAAAGAACGAGGGCAC | | |
| | | CTGTGGCCCTGCTATGCTTGA | | |
| prey84354 | 1050 | TTCTGGGCAGGGCTCCATGCCTGCATGGCTGTACGATCTACTTCCTT | 1051 | VNGVILGRAPCLHGCTIYFL*LHP |
| | | TTGCTAAAGGGAATATTTTATACAGACAGCCTTGAATGACTTGAATG | | AKGNILYRNSLE*LECLTQNTDIL |
| | | TCTACAGATATTTTAAATAGAAACATACTGAACATACTGAACATTAG | | NRNILNKPLQH**CPTANKSNRFR |
| | | TAACAAATAAATCAAATCGGTTTAGGTGTCATTTGTTGGATATATTC | | CHLLDIFIRKKTNLRPXXXXXXXX |
| | | ATTAGACTTGAGGCCNNNNNNNNNNNNNNNNNNNNNNNNNNNNNNNN | | XXXXXXXXXXXXXXXXXXXXXXXX |
| | | NNNNNNNNNNNNNNNNNNNNNNNNNNNNNNNNNNNNNNNNNNNNNNN | | XXXXXX |
| | | NNNNNNNNNNNNNNNNN | | |
| prey84355 | 1052 | GAAACCCCTATAGAGTGTACTGTTCTAGCAATTATCTAAACTCTCAG | 1053 | RLTFETPIECTVLAIIL*TLRNLS |
| | | AATTCCAGGGAGTTTTGGGAGTGAGAGGATTGGGATTATTGGGAGAG | | REFWE*EDWD*LGENNYSYIFILF |
| | | AACAATTACAGTCTCTTCATGTGACTCGTTTAGCCTTCCTAGTAAAT | | M*LV*PS*VKCCHCSLRFLKMVDL |
| | | GCTGTCATTGTTTCCTGAAAATGGTTGACTTGCCCCATAAAGGTATT | | PHKGIHSDTVIQLMIIIAVTQQH* |
| | | CACAGTGATACAGTGATGATAATAATAGCAGTTACACAACAGCATTA | | WSVLSLKNYYTFFQLKLRN*ICNL |
| | | ATGGAGTGTTTTAAGTTTAAACACATTCTTTCAGTTGACTCAGATTL | | SYKCGSLLNFV*LVCSQDLIPDLL |
| | | AATATGTAAATCTCTCTTACAAATTGCTCAACTTTGTATAGCTAGTG | | TVKSVYLFIMPECFIAAHWIRMMR |
| | | TGTAGTCAGGACTTGATTCCAGACCTCTAGTCAGTGTACCTTTTCAT | | EC*KRV*TLVLNL |
| | | TATGCCAGAGTGTTTCATTGCAGCCCACTGGATGAGGGAGTGTTAGA | | |
| | | AGAGGGTATACTTTAGTTTTGAATTTAAG | | |
| prey84356 | 1054 | GGAATTTTTAGGCAAGCTCTCAAGACTTGAAGAGTTGGAAATTCTTG | 1055 | YTTMEIFKASSQDLKSWNSWHLND |
| | | GATGCCTGAAGTTTGTTTTCAAAAAGCAATATTTCATTGCTCTTTTC | | A*SLF*KSNISLLFSIVKQLLSKY |
| | | AATTCTGCTCAGCAAATATTACTCATTTTTTTAAGTTGAAATTAAAC | | YSFF*KVELKGNIFLKPKIKMILP |
| | | GGAAATAAACCAAAATAGATCAAAATGATCCCAGTTTTAAGGGATTT | | VLRDFAYFGRIKLKC*NC*ITENR |
| | | TGCCTATTTTGGACTAAAATGTTAAAACTGTTGAATTACAGAGAACA | | CTLRHNFKQIFKIRQVNICV*AQ* |
| | | GATGTACTCTGAGACATCAAATATTTAAAATAAGGCAGGTTAACATT | | ICIKGKHIKEIYTC*IM*LKNIF* |
| | | TGCGTTTAGGCACAATAAATCTGGGAAGCACATCAAGGAAATATATA | | KPLKKKNNLMGTLSIVDRN*CIIV |
| | | CATGTTGAATAATGTAACTGAAAAATATACCACTTAAAAAGAAAAAT | | SSQSLYEKLKLSSPLMSMNLYFYI |
| | | AATTTGATGGGTACTTTAAGCATTGTAGATAGAATAATAGTGTCCTC | | CCLVKLSSVL*VIQIYV*YC*ILH |
| | | CCAGTCTTTGTATGAAAAATTAAAAACTCTCTAGTCCTTTGAATTTA | | QRA*IYCLLYFVTCAYLKKLTT |
| | | TACTTCTACATTTGTTGCTTAGTAAAAATTATCCTCAGTACTTTGGA | | |
| | | TTTATGTTTAATATTGTTAAATTCTTCATCAGAGAGCATAAATATAT | | |
| | | TGTTTTGTAACTTGTGCATATCTAAAGAAACTAACAACGA | | |
| prey93040 | 1056 | TAATTTTATTCTGGGAATTTCAGTCATTCACCATAAAAAGGTGATCA | 1057 | LLGWF*FYSGNFSHSTIKR*KVSV |
| | | GATGTGCGTGTAGTGGTCTGTATAATAAAGACTGAAGGAAGCAACCA | | RCACSGLYNKD*RKQPNQLRDFL* |
| | | AACTTTCTCTAAAGGCAGAGTAATGCTAAGGGCAGTGGCAGTGACAA | | RQSNAKGSGSDKSGGKSLSFGRQN |
| | | AAGTCTGTGATCATTTGGGAGACAGAATAGGAAGTGCTTGGTCATGA | | RKCLVMRCERRGEVEGKQSCSGSA |
| | | GATGTGAAGTAGAAGGAAAACAGAGCTGCTCAGGCTCTGCTGGGGAA | | GEDWVCS |
| | | AGACTGGGT | | |
| prey84362 | 1058 | CTAGGGCGCCCCAGCAATGGGCCACTTTTGCTAGAATATGGTATCTC | 1059 | MSSFSRAPQQWATFARIWYLLDGK |
| | | TAAATGCAGCCACCTGGCAAACTTGCTGCTATGGCATCTATAAGACT | | MQPPGKLAAMASIRLQGLHKPVYH |
| | | TCAGGGACCTGTGTACCATGCACTGAGTGACTGTGGGGATCATGTTG | | ALSDCGDHVVIMNTRHIAFSGNKW |
| | | TTATAATGAACATTGCATTTTCTGGAAACATGGGAAACAAAAGTATA | | EQKVYSSHTGYPGGFRQVTAAQLH |
| | | CTCTTCGCATACTGGTGGATTTAGACAAGTAACAGCTGCTCAGCTTC | | LRDPVAIVKLAIYGMLPKNLHRRT |
| | | ACCTGAGGGATCCAGTGGCACTAGCTATTTATGGCATGCTGCCAAAA | | MMERLHLFPDEYIPEDILKNLVEE |
| | | AACCTTCACAGAAGAACAATGATGATCTTTTTCCAGATGAGTATATT | | LPQPRKIPKRLDEYTQ |
| | | CCAGAGATATTCTTAAAGAAATTTAGTAGCTCAACCACGAALAATAC | | |
| | | CTAAACGTCTAGATGAGTACACACAAAG | | |
| prey84369 | 1060 | ATGCAAAATGCTATTTTACATTTTTTGACTATTTTGGCATTTTATAA | 1061 | YFL*NAKWYFTFFDYFGIL*NKPT |
| | | AACATTTTTGGAGACATTTTTAAAGAAGCCAATCTTTTCTAAAATAA | | FLETFLKKPIFSKIKLIHQKLET* |
| | | AATTAATACTAGAGACATAAATGTCGTATACATCGTTTACTGAATCC | | MSYTSFTESYSNL*SLCTSMLDYL |
| | | TACAGTAATCTGTGTACATCAATGTTAGATTACCTAAACATATAAAG | | KHNKVSILRYI*YTKVQNISRQTK |
| | | TTTCTATTTTACGTTATACAAAAGTCCAATATATCCAGACAGACAAA | | IVAATLKNNVCLANVAHACKPNTLEG |
| | | AAATTGTGGCTGCCACTTTCTTTGTCTGGCCATGGTGGCTCATGCCT | | |
| | | GTAIAGCCCAACACTTTGGIAGGC | | |
| prey84370 | 1062 | AGGAGCCGAATGGCTCCTCCCTGAAGAACATAGAGAAGTATCTCAGA | 1063 | IEGLEEPNGSSLKNIEKYLRSQSD |
| | | ACCACCAACAACCCAGCCTTTCAGCAGCGGCTGCGACTGGATGGGAG | | LTSTTNNPAFQQRLRLGAKRAVNN |
| | | GTTACTGAAAGACGGACCGCAGTACAGGGTCAAAGGGGCACCTCAGT | | GRLLKDGPQYRVNYGSLDGKGAPQ |
| | | ATCCCAGTGCATTCCCATCCTCGCTCCATGAGAAAGACCAGCCCCGT | | YPSAFPSSLPPVSLLPHEKDQPRA |
| | | GCTGATCCCATTCCAATATGAATCAAATCGTGAAAGAACCAGAAGAA | | DPIPICSFCLGTKESNREKKPEEL |
| | | CTCCTCTCTGGACACCCATCCTGTTTGAAATTTTGTCCTGAATTAAC | | LSCADCGSSCHPSCLKFCPELTTN |
| | | ATGGCAGTGCATCGAATGCAAGACATGCACTCCCTGTAGAGATATGC | | VKALRWQCIECKTCSACRVQGRNA |
| | | TTTTTTGTGATTCCTGTGATAGAGGATTTCATATTTCCAGAATGCCA | | DNMLFCDSCDRGFHMECCDPPLSR |
| | | AAAGGGATGTGGATTTGCCAAGTCTGCAAACTACTTCATGAGAAGCT | | MPKGMWICQVCRPKKKGRKLLHEK |
| | | GCACAATAAAACGACGATCGAAAAATAAATTAAAGCAACGATTGTTG | | AAQIKRRYAKPIGRPKNKLKQRLL |
| | | TCTCTAACCAGATTCACAGGAAGGGGGTCACCTGGTAGGGGTCAAAA | | SVTSDEGSMNAFTGRGSPGRGQKT |
| | | GACTTCTGGTCATGCTGCATCTGGGAAGGACTCAAGCAGCAGATCCC | | KVCTTPSSGHAASGKDSSSRLAVT |
| | | TGGTGCCACCACCAAATCACCACCACCTCCACCTAAGTTAACAAGAA | | DPTRPGATTKITTTSTYISASTLK |
| | | AACCAAAGGGCTCATTCATCGCCTTACTATGGTCGCAGATCACGAGG | | VNKKTKGLIDGLTKFFTPSPDGRR |
| | | TGAAATTATAGACTTTTCAAAGGTCTCTCAGAAACAGTCATGCACTT | | SRGEIIDFSKHYRPRKKVSQKQSC |
| | | CTCATGTGTTGGCAAAACCTCCACCTTCTTCACTTCCACCCCCAACC | | TSHVLATGTTQKLKPPPSSLPPPT |
| | | CCCATCCAAAAGTCCAGCACGGCCACTTCTTCTCCCTCTCCCCAGAT | | PISGQSPSSQKSSTATSSPSPQSS |
| | | GCCCTCCCTGAGCAGCCTTACCACTAACAGCCAGCTCAATCATACTA | | SSQCSVPSLSSLTTNSQLKALFDG |
| | | TACCACTCAGGGACAGTCTCGCAAAAAGGGACAAACGTATGCGTCGT | | LSHTIPLRDSLAKRDTPSYAPPKR |
| | | AAAACTGAATTATCTTCCACGGCAAGCAAAAGAGATATTAGAAGTCG | | MRRKTELSSTAKSKAHFFGKRDIR |
| | | GTTTATTTCTCACTCCTCTAGAGGAAGTATTTTTAAAGCAATTGCTC | | SRFISHSSSSSWGMARGSIFKAIA |
| | | ACTTCAAGCGAAGGATGCTACCCAGATTAAAATATAAACTGACCCCT | | HFKRTTFLKKHRMLGRLKYKVTPQ |
| | | CAGAAGGGGAGCTTGACAGACGGAAGGATTAAACCTGATCAGCAAAA | | MGTPSPGKGSLTDGRIKPDQDDDT |
| | | CATCAAACAAGAAAGTGCAGATGTAAATGTGATTCCAGAGGATTTGG | | EIKINIKQESADVNVIGNKDVVTE |
| | | ATGTTTTTAAGCAGGCCCAGGAACTTTCTTGTGGGGTGGAAGACTGT | | EDLDVFKQAQELSWEKIECESCVE |
| | | GGCCGGTACCCTTCTGTGATTGAAACCTGCTACTCCTCGCCTTACCC | | DCCRYPSVIEFGKYEIQTWYSSPY |
| | | ACAGGAATATGCAAGAATTCTGTCTTAAATATATGAAAAAGTAAATA | | PQEYARLPKLYLCEFCLKYMKSKN |
| | | TTTTGCGATGGTTTCATCCTCCAGCAAATGAAATTTACCGAAGGAAT | | TLLRHSKKCGWFHPPANEIYRRKD |
| | | GATGGGAATATGAGCAAAATTTATTGCCAAAACCTTTGCGACCACAA | | LSVFEVDGNMSKIYCQNLCLLAKL |
| | | AACGTTGTATTATGATGTCGAGCCATTCCTTTATGAAAAGGGCTGTC | | FLDHKTLYYDVEPFLFYVLTKNDE |
| | | ATCTGGTTGGATACCTCTCTAAGGATAATGTCTCCTGCATAATGATC | | KGCHLVGYLSKEKLCQQKYNVSCI |
| | | ATGCCCCAGCACCAAAGGTTGATTTCACCTATTTGCTTTCTAGAAGA | | MIMPQHQRQGFGRFLIDFSYLLSR |
| | | GAAGGCCAAGTCTCCGATCTGGGCCGTCTCTCCTACCTGGCATATTG | | RECQAGSPEKPLSDLGRLSYLAYW |
| | | GAATTGGAGTATCTCTACCACCACCATGAGAGGCACATCAGCATCAG | | KSVILEYLYHHHERHISIKAISRA |
| | | GCATTCGGGCATGTGCCCACATGACATTGCCACCACTCTGCAGCACC | | TGMCPHDIATTLQHLHMIDKRDGR |
| | | TCCACATGAAGATGGCAGATTTGTCATCATTACACGGGAAAAGTTGA | | FVIIRREKLILSHMEKLKTCSRAN |
| | | TATTGAGCCACATAAAACCTGTTCCAGAGCCAATGAACTTGATCCAG | | ELDPDSLRWTPILISNAAVSEEER |
| | | ACAGTCTGAGGTGGACCTTTCTAATGCTGCAGTGTCTGAAGAGAGCG | | EAEKEAERLMEQASCWEKEEQEIL |
| | | AGAAGCTGAGAAGAGGCTGGGAACAAGCTAGCTGCTGGGAGAAGGAG | | LSTRANSRQSPAKVQSKNKYLHSP |
| | | GAACAAGAAATCCTGTCAACTAGAGGCAATCACCTGCAAAAGTACAT | | ESRPVTGERGQLLELSKESSEEEE |
| | | CGAATAAATATTTGCATTCCCCGAGTCACAGGGGAGCGAGGGCAGCT | | EEEDEEEEEEEEEEEEDEEEEEEE |
| | | GCTGGAGCTGTCTAAGAGAGCAGTGGGAGGAGGAGGACGAGGAGGAG | | EEEEEEENIQSSPPRLTKPQSVAI |
| | | GAAGAAGAGGAGGAAGAAGAGGAAGAGGAGAAGAAGAGGAAGAAGAA | | KRKRPFVLKKKRGRKRRRINSSVT |
| | | GAAGAAGAAGAAGAAAATATTCAAAGCTCTCCCCGAAACCACAGTCA | | TETISETTEVLNEPFDNSDEERPM |
| | | GTTGCCATAAAGAGAAAAGAGGCCTTTTGTACTAAAGATCGTAAACG | | PQLEPTCEIEVEEDGRKPVLRKAF |
| | | CAGGAGGATCAACAGCAGTGTAACAACAGAGACCATTTCAGAGTACT | | QHQPGKKRQTEEEEGKDNHCFKNA |
| | | GAATGAGCCCTTTGACAACTCAGATGAAGAGAGGCCAATGCCACAGC | | DPCRNNMNDDSSNLKEGSKIDNPE |
| | | CTGTGAGATTGAAGTGGAGGAGATGGCAGGAAGCCAGTCCTGAGAAA | | PLKCKQVWPKGTKRGLSKWRQNKE |
| | | GTCAGCCTGGGAAGAAAAGACAACAGAGGAAGAGGAAGCAAAGACAA | | RKTGFKLNLYTPPETPNEPDEQVT |
| | | TCAAATGCTGACCCTTGTAGAAACAATATGAATGATGATTCAAGTAA | | VEEQKETSEGKTSPSPIRIEEEVK |
| | | CTTGAAAAAGACAATCCCGAACCTCTAAAGTGCAAACAAGTGTGGCC | | ETGEALLPQEENRREETCAPVSPN |
| | | AAAAGGAACAAATCTAAGTGGAGGCACAAGAGAGGAAGACCGGATTT | | TSPGEKPEDDLIKPEEEEEEEEEE |
| | | AAACTGAATTTCCAGAAACACCCATGGAGCCTGACGAGCAGGTACAG | | EEEEEEEEGEEEEGGGNVEKDPDG |
| | | TGGAAGAAACAGAGAAGGAAAAACCAGCCCCAGTCCCATCAGGATTG | | AKSQEKEEPEISTEKEDSARLDDH |
| | | AGGAGGAGGTCAAAGGAAACCTGTTGCCTCAAGAGGAAAACAGAAGG | | EEEEEEDEEPSHNEDHDADDEDDS |
| | | GAAGAAACATGTGCCCCTGTAAGTCACCAGGTGAAAAACCAGAAGAT | | HMESAEVEKEELPRESFKEVLENQ |
| | | GATCTCATCAAACCTGAGGAAGAGGAAAGGAGGAAGAGGAAGAAGAG | | FTFLDLNVQPGHSNPEVLMDCGVD |
| | | GAAGAAGAGGAAGGGGAAGAAGAAGAAGGAGAGAAAAAGATCCAGAT | | LTASCNSEPKELAGDPEAVPESDE |
| | | GGTGCTAAAAGCCAAGAAAAAGAGGAACCAAGAAAAAAGAAGACTCT | | EPPPGEQAQKQDQKNSKEVIDTEF |
| | | GCACGTTTGGATGATCACGAAGAGGAGGACGAAGAGGATCCCACAAC | | KFGNPATMEIDSETVQAVQSLTQE |
| | | GAGGACCATGATGCCGATGACGAGGATGACAGCCACATGGAGTTGGA | | SSEQDDTFQDCAETQEACRSLQNY |
| | | GAAGGAAGAGCTGCCCAGAGAAAGCTTCAAAGAAGTACTGGAAAACC | | TRADQSPQIATTLDDCQQSDHSSP |
| | | ATTAGACCTTAAGTGCAGCCTGGTCACTCGAACCCAGAGGTCTTAAT | | VSSVHSHPGQSVRSVNSPSVPALE |
| | | GGACACCTGACAGCTTCTTGTAACAGTGAGCCCAAGGAGCTTGCTGG | | NSYAQISPDQSAISVPSLQNMETS |
| | | GGACCCTGCGAATCTGACGAGGAGCCACCCCCAGGAGAACAGGCACA | | PMMDVPSVSDHSQQVVDSGFSDLG |
| | | GAAGCAGGACCACAAGGAAGTCGATACAGAGTTCAAAGAGGGAAACC | | SIESTTENYENPSSYDSTMGGSIC |
| | | CAGCAACCATGGAAATCCTGTCCAGGCCGTTCAGTCTTTGACCCAGG | | GNGSSQNSCSYSNLTSSSLTQSSC |
| | | AGAGCAGCCAACAGGACGACATTGTGCCGAGACTCAAGAGGCCTGTA | | AVTQQMSNISGSCSMLQQTSISSP |
| | | GAAGCCTACAGACTACACCCGTGCCCACAGATTGCCACCACGCTCCA | | PTCSVKSPQGCVVERPPSSSQQLA |
| | | CGATTGCCAACAGTCGGACCACAGTAGCCCGTCCACTCCCATCCTGG | | QCSMAANFTPPMQLAEIPETSNAN |
| | | CCAGTCCGTACGTTCTGTCAACAGCCCAAGTGGGAAAACAGCTACGC | | IGLYERNGQSDFGAGHYPQPSATF |
| | | CCAAATCAGCCCAGATCAAAGTGCCATCTCAGTGCCAACATGGAAAC | | SLAKLQQLTNTLIDHSLPYSHSAA |
| | | CAGTCCCATGATGGATGTCCCATCACTTTCAGATCATTCATAGACAG | | VTSYANSASLSTPLSNTGLVQLSQ |
| | | TGGATTTAGTGACCTGGGCAGTATCGAGAGCACAACTGAGAACTAAG | | SPHSVPGGPQAQATMTPPPNLTPP |
| | | CAGCTACGATTCTACTATGGGAGGCAGCATCTGTCGAACGGCTCTTC | | PMNLPPPLLQRNMAASNIGISHSQ |
| | | GCTCCTATAGCAACCTCACCTCCAGCAGTCTGACACAGAGCAGCTGT | | RLQTQIASKGHISMRTKSASLSPA |
| | | CCTTGTCCAACATCAGCGGGAGCTGCAGCATGCTGCAGCAACCAGCA | | AATHQSQIYGRSQTVAMQGPARTL |
| | | TCACTGCAGCGTCAAGTCTCCTCLAIAGGCTGTGTGGTGGAGAGGCC | | TMQRGMNMSVNLMPAPAYNVNSVN |
| | | TCCGAGCTGGCTCAGTGCAGCATGGCTGCTACTTCACCCCACCCATG | | MNMNTLNAMNGYSMSQPMMNSGYH |
| | | CAGCTGAGACGAGCAACGCCPACATTGGCTTATACGAGCGATGGGTC | | SNHGYMNQTPQYPMQMQMGMMGTQ |
| | | AGAGTGGCATTACCCGCAGCCGTCAGCCACCTTCAGCCTTGCCAACT | | PYAQQPMQTPPHGNMMYTAPGHHG |
| | | GCAGCACTTATTGATCATTCATTGCCTTACAGCCATTCCGCTGCTCT | | YMNTGMSKQSLNGSYMRR* |
| | | GACTTCCCTCTTTGTCCACACCATTAAGTACACAGGGCTTGTTCACT | | |
| | | TTCTCCGTCCCTGGGGGACCCCAGCACAGCTACCATGACCCCACCCC | | |
| | | CCACCATGAATCTGCCGCCGCCTCTTTTGCACGGACATGGCTGCATC | | |
| | | ACTCACAGCCAGACTGCAACCCAGATTGCCAGCAAAAGGGCCACATC | | |
| | | TCCAAGTCAGCGTCTCTGTCACCAGCCGCTGCCACCCATCAGTCACA | | |
| | | AAAATCTACCAGACTGTAGCCATGCAGGGTCCTGCACGGACTTTAAA | | |
| | | CGATGCAAGAGGCAGTGTGAAACCTGATGCCAGCGCCAGCCTACAAT | | |
| | | GTCAAACTCTGTGAAACATGACTCTCAACGCCATGAATGGGTACAGC | | |
| | | ATGTCCCAGCCAATGATGAACAGTGGCAATCATGGCTATATGAAATC | | |
| | | AAACGCCCCAATACCCTATGCAGATGCAGATGGGCACCCAGCCATAT | | |
| | | GCCCAGCAGCCAAAATGCAGACCCCACCCCACGGTAACACGGCCCCC | | |
| | | GGACATCACGGCTACATGACACAGGCATGTCCAACAGTCTCTCTACA | | |
| | | TGAGAAAGGTAG | | |
| prey84378 | 1064 | GCAAAGAGAAGGTGATCCGATTGGTGGTAGTGGAGAGAAAGTGGGTG | 1065 | ITVIQAREGDPIGGSGEKWVSYGY |
| | | ATTGAAAGGTAGAGCCAAATGAGATTTGCTGATAGTTTGGATGTGAT | | I*KVEPMRFADSLDVICVWEKRYQ |
| | | ATGAGATATCAGGGTGGATACAAAAAGTTTTTTGCCTACACAGCTCG | | GWIQSFLPTQLEVWNSCQLK*RIL |
| | | AGTTTGTCAACTAAATGAAGATATTGTGCAAAGAGGAAATTTTTTTT | | CKEEFFF*RGKGTKSLCMLSLRCD |
| | | TTAAGGGCCAAGAGCTTGTGCATGTTIAAACTTTGAGATGCGATTAG | | *ASMWRY*IG |
| | | GCATCCATGTGGAGAGGCGG | | |
| prey84380 | 1066 | CTTTNNNGNGGGGAGGGCAGGGGCCAGATTTGCTNCTGTGCANCTTN | 1067 | GXXFFFXXGKGRGQICXCAXLXXX |
| | | AGNACAAGTTAGNTGTGGAGGCCTAGNTAGGTTCAGATTTNNTTACT | | XXTS*XWRPX*VQIXLLIKXSARX |
| | | GCAAGAATNCTTTNNGGTGNTGNCNTNTNTACTTCCATCNGCTANAG | | LXGXXXXTSIXXRHIXXYVSL*XY |
| | | GATGTCTCTCTATGANATTACCANNCACTGATACCATTCTCTATATC | | XXLITIVYIYYXTRDXPGCRXXPX |
| | | TCTAGGGACNTACCTGGTTGTCGAAAATNATNCCATNAAANGNANNA | | KXXIXHEXVXXXTXV*XYYSTRXL |
| | | TATNCCAGATNNTTNTCACCATNGTCTAANCTATTATTCCACTAGGG | | PXLXY |
| | | NCCTTCCCTNGNA | | |
| prey51316 | 1068 | CCCTGTGGGTGCTCCAGGAGATGCAGGACAAAAGAGGAGATCCGGGT | 1069 | PGTPGPVGAPGDAGQRGDPGSRGP |
| | | TAGGACACCTGGGTCGAGCTGGAAAACGTGGATTACCTGGACCCCAA | | IGHLGRAGKRGLPGPQGPRGIDKG |
| | | AGGAAAGGTGATCATGGAGACCGAGGCGACAGAGGTCAGAAGGGCCA | | DHGDRGDRGQKGHRGFTGLQGLPG |
| | | CAGAGGTCTTCAGGGTCTTCCTGGCCCTCCTGGTCCTGGTGAAACAG | | PPGPNGEQGSAGIPGPFGPRGPPG |
| | | GAAGTGCTGGACCATTTGGCCCAAAGAGGTCCTCCAGGCCCAGTTGG | | PVGPSGKEGNPGPLGPLGPPGVRG |
| | | TCCTTCAGGTAAACCCTGGGCCACTTGGGCCATTGGGACCTCCAGGT | | SVGEAGPEGPPGEPGPPGPPGPPG |
| | | GTACGAGGCAGTGTAGGAGGGCCCTCCTGGTGAGCCTGGCCCACCTG | | HLTAALGDIMGHYDESMPDPLPEF |
| | | GCCCTCCGGGTCCCCCTGACAGCTGCTCTTGGGGATATCATGGGGCA | | TEDQAAPDDKNKTDPGVHATLKSL |
| | | CTATGATGAAAGCATGCCAGATCCAGTTTACTGAAGATCAGGCGGCT | | SSQIETMRSPDGSKKHPARTCDDL |
| | | CCTGATGACAAAAACAAAACCGACCCAGGGGAAGTCACTCAGTAGTC | | KLCHSAKQSGEYWIDPNQGSVEDA |
| | | AGATTGAAACCATGCGCAGCCCCGATGGCTCCCGCACGTGTGATGAC | | IKVYCNMETGETCISANPSSVPRK |
| | | CTAAAGCTTTGCCATTCCGCAAAGCAGAGTTGATCCTAACCAAGGAT | | TWWASKSPDNKPVWYGLDMNRGSQ |
| | | CTGTTGAAGATGCCATCAAAGTTTACTGCAACATGTATTTCAGCAAA | | FAYGDHQSPNTAITQMTFLRLLSK |
| | | CCCATCCAGTGTACCACGTAAAACCTCTCCTGACAATAAACCTGTTT | | EASQNITYICKNSVGYMDDQAKNL |
| | | GGTATGGTCTTGATATGAACAGAGGGAGACCACCAATCACCTAATAC | | KKAVVLKGANDLDIKAEGNIRFRY |
| | | AGCCATTACTCAGATGACTTTTAGAACCCTCCCAGAACATCACTTAC | | IVLQDTCSKRNCNVCKTVFEYRTQ |
| | | ATCTGTAAAAACAGTGTAGCTAAGAACCTCAAAAAAGCTGTGGTTCT | | NVARLPIIDLAPVDVGGTDQEFGV |
| | | CAAAGGGGCAAATGAGGAAATATTAGATTCCGGTATATCGTTCTTCA | | EIGPVCFV* |
| | | AGACACTTGCGTGGGCAAGACTGTCTTTGAATATAGAACACAGAATG | | |
| | | TGGCACCATAGATCTTGCTCCTGTGGATGTTGGCGCCACAGACCAGG | | |
| | | AATTCGGCGTCCAGTTTGTTTTGTGTAA | | |
| prey84382 | 1070 | TNAGCTNGNAATNTTTGAGGATGCTNGTNTCACGGANCNNCNGGGAN | 1071 | GEXXYXLXXFEDAXXTXXXGXXWD |
| | | CAGCAAGGANGNTNCTCTTNACTACANNNGGNGCTGTGCCTGNCCNT | | SKXXXLXYXXXCAXPSXG*XFXLX |
| | | CCTTTTCTNACTGNCNTANNNTGCATCNCGCATCNNGGAAGATCACT | | XXASRIXEDH*XFXXLTLCXSF*Q |
| | | GANGNTTTCGCTTTGTNNATCTTTCTGACAAGGAGAATNAACAGACC | | GEXTDXMSXXXXWSRXXCNPXTXT |
| | | NGATGTCTGANNGTCTCGCTNNNTTTGCAACCCCNTGACANACACTN | | XLXXXSXXSXDSXLPXYSXLDTLQ |
| | | ACCTNCNTGNGNTCTCTTNGATTCNTGNCTCCCNCNCTACTCNNCGC | | XXAXPXAGAAXREGQAXRASSPXX |
| | | TTGACACCTTGCAGGNTCNTTNGCGGGNGCAGCCNGACGNGAGGGCC | | DLMEGI |
| | | AGGCTGNAAGGGCCTCNTCNCCNAAATGGAAGGAATTC | | |
| prey84385 | 1072 | CCCATAACTAATAGACTTCAGTTTTTCGGGTTTTTTTAGACGGAGTT | 1073 | CSPHPITNRLQFFGFFATEFRSVA |
| | | CCCAGNNNNNNNNNNNNNNNNNNNNNNNNNNNNNNNNNNNNNNNNNN | | XXXXXXXXXXXXXXXXXXXXXXXX |
| | | NNNNNNNNNNNNNNNNNNNNNNNNNNNNNNNNNNNNNNNNNNNNNNN | | XXXXXXXXXXXXXXXXXNFNLLYF |
| | | NNNNNNNNNNNCAATTTTAATTTACTTTATTTTTAAATTNNNNNNNN | | *IXXXXXXXXXXXXXXXXXXXXXX |
| | | NNNNNNNNNNNNNNNNNNNNNNNNNNNNNNNNNNNNNNNNNNNNNNN | | XXXXXXXXXXXXXXXXXXXYLALF |
| | | NNNNNNNNNNNNNNNNNNNNNNNNNNNNNNNNNNNNNNNNNNNNNNN | | FL*NKVLLXGXGWSAX |
| | | NTACCTGGCCCTTTTTTTTTTATNTTGCTNTGNGGCCNAGGCTGGAG | | |
| | | TGCAGNC | | |
| prey84395 | 1074 | TTCTTCAGGTGTTCACCATGTCCCAGTAGAGCTGAGCCACACGTATC | 1075 | SLAFFFRCSPCPSRAEPHVSHIGA |
| | | ACCACTGTGTTAGCCATTATTTAGCCATGCATGAAAAATCCTTACAT | | TVLAII*PCMKNPYIFSLHQISKF |
| | | CTTCAGATCTCTAAGTTTAATATGTTCTTTATCATGCTTTCTTAAAA | | V*HKCRDFPGIFPSQWLESPILNG |
| | | CAAGGGATACGTGAAGTCTGACACAAATGCAGAGATTTCCCAGGAAT | | ETTLFTIVKTKKVLKDRHYCCFPF |
| | | TTTTCCTAGTCAGTTCCAATTCTAAATGGGGAGACAACATTATTTAC | | *EVMEIPCGFTYI*TYIHIDMQGR |
| | | AATTGTAAAAACCAAAAAGACAGGCATTATTGTTGCTTTCCATTTTG | | H*E*KEEKFKYKKNQNLCLIVKDN |
| | | AGAGGTCATGGAAATCCCATGTATATATAAACATATATACATACAGA | | FIQHKDRTKCHNVTNSRLRFSSTS |
| | | TATGCAGGGGAGACATTAAGAATGAAATTTAAGTACAAAAAAAATCA | | NIWLKGLLLGVFGQILVISFLK*V |
| | | AAACCTATGTTTAATAGTAAAGGACAATTTAAAGATAGAACCAAATG | | IL*FHPMILL*KVEETETRRID*F |
| | | TCACAATGTCACAAATTCAAGATTAAGATTTTCCTTGGTTAAAGGGT | | FKGSRTFKIKVKIF |
| | | TTATTGCTGGGAGTTTTTGGCCAAATTCTAGTGATTAAGTAAGTGAT | | |
| | | CCTCTAGTTCCATCCCATGATTCTTCTCTAAAAAGTAGAAATAAGAA | | |
| | | GGATAGACTGATTTTTTAAGGGATCCAGGACTTTTAAAATCAAATTC | | |
| | | T | | |
| prey84408 | 1076 | TATTTTTTTCATGATACTTGTAGAAAAAATGGGCTTCATGTATAAAA | 1077 | YFL*YFFHDTCRKNGLHV*KAKKY |
| | | AGATATTTTTTTGTATGAAAACGTTTACCAGGAATAGTAATAGTAAT | | IFLYENVYQE****YIXTFPLIPS |
| | | ACATAANCCTTATTCCTTCANATTTTTAAGTTTACCTCATTATAGAG | | XF*VYLIIEAIYSFNGNTNLLVML |
| | | GCAATTTACTCCAATACCAATCTCCTAGTAATGCTGGGTAATTGGTA | | GNWYKDFNSWAQTFRSPLYIF*LH |
| | | TAAAGACTTCAATAGTTCCTTCAGGTCCCCCTTATATATATTTTAGC | | FK*PNATY*ICSDGVSL*NFWRCH |
| | | TGCATTTCAAATGACCGAATCCAATTTGCAGTGATGGTGTGTCTCTG | | LMLLESSAQVLKLL |
| | | TAGAACTTCTGGCGATGCCATTTGATGTCTTCAGCCCAGGTGCTTAA | | |
| | | GTTGCTC | | |
| prey84409 | 1078 | GGCACTGGAGTTTGTTGGATGTGAGGGGAATGCCCACTGGGGCCAGG | 1079 | ATTAGDWSLLDVRGMPTGARVGKG |
| | | GGAGGTTGGTGCCAATGTGGTGCTGGAAGTAGACCCCTCCTGGGTCT | | GWCQCGAGSRPLLGLEHSLPQALS |
| | | GGAACACCAGGCTCTTTCCACCTGGGGATGTTGTGACAACATCCAGC | | TWGCCDNIQLWCLGYLCGDKTDLV |
| | | TGTGGTGTCTGTGTGCTGACAAGACAGACCTGGTCAGGGGGTGGGTG | | RGWVANTSPGWAEATLLFGPRPGE |
| | | GCAAACACCTCACCTGAGGCCACCTTGCTGTTTGGCCCAAGGCCAGG | | APPFPPLPWPRGHSFLGFV*VFHR |
| | | TGAGGCCCCTCCCTTTCCACCGCCCCGTGGCCATTCATTCTTGGGGT | | EQQRDMGVG*SVYLLCRFFWK*PL |
| | | TTGTCTGAGTCTTCCACAGAGAGCAGATGGGAGTTGGGTGATCAGTG | | AAAICKAGIGLPVFSNNQDPILSV |
| | | TATTTACTTTCCAGATTTTTCTGGAAATAACCTGCTATTTGCAAGGC | | PVLLFASALLLSPGWSPASLLAFA |
| | | TGGGATTGGCCTCCCTGTCTTTTCCAACAATCAGGAGTCTGTTCCTG | | CSISSL |
| | | TTCTTCTCCCAGCCTCTGCCCTCCTTCTGTCTCCTGGGTGGCGCTCC | | |
| | | TGGCCTTTGCATGTTCCATCTCTTCTCTTTC | | |
| prey84414 | 1080 | TTTTGTTCTATCACAGAATACCACAGNACTGGGTAATTTATAAAGAA | 1081 | PSNVCFVLSQNTTXLGNL*RTEIY |
| | | ATTTCTCACAGTTCTGGAGGCTGGGAAGTCCAAGATCAAACCTCATC | | FSQFWRLGSPRSNLIKL*ALIA1F |
| | | AAATTGCATTTTGCAGTCCTCTGGAGGTCAAGAGAAGCTACCTGAAG | | AVLWRSREAT*SLFFKGLNPIHVG |
| | | CCTNTTTTTTAATCCCATTCATGTAGGGGTAAGCTCTCATGGGCTGA | | VSSHGLIIL*KPYLLVLSLCQHMN |
| | | TCATCCTTTAAAAGCTAGTACTATCACTTTGCCAACACATGAATTTC | | FKGTHSYCCRCLNSLLLLIXVTTL |
| | | AAAGGCACCCATTCATACTGCTTAAATAGTTTACTTCTTTTAATTNG | | ***KLTFIEHFTLCRXHSICFXI |
| | | AGTGACAACTCTGTAATAATAGAAGTTGAGCATTTTACTTTGTGTCG | | |
| | | CNACCATTCTATATGCTTTTGNATAT | | |
| prey84482 | 1082 | GCATTGTCTAGGATTCTTCTAGGATTTTTATGGTTTTAGGTTAACAT | 1083 | LCPQWHCLGFF*DFYGFRLTFKSL |
| | | TAATCCATTTTGAATTAATTTTTGTGTAAGGTGTAAGGAAGGGATCC | | IHFELIFV*GVRKGSSFSFLHMAS |
| | | AGTTTACATATGGCTAGCCAGTTTTCCTACCACTGTTTGTTAAATAG | | QFSYHCLLNRESFSHFLFLSGLSN |
| | | GGAATCTTTTTTGTTTTTGTCAGGTTTGTCAAACATTAGATGGTTGT | | IRWL*MCVLFLRPLFCSIGLYICF |
| | | AGATGTGTGTGTTATCTCTGTTCTGTTCCATTGGTCTATATATCTGT | | VPVPCHFGYCRIXX*FEVR*CDAS |
| | | TTTGTACCAGTACCATGCCACTGTAGGATTGNAGNATAGTTTGAAGT | | TFHSFCLXLPWQCRL |
| | | CAGGTAGTGTGATGCCTCCACCTTCTCCTTANGATTGCCNTGGCAAT | | |
| | | GCAGGCTC | | |
| prey84416 | 1084 | GNCCACANGTATGGCATTAAANANCAGTGCAGGANGTNCNTAAATAC | 1085 | AGXXAXTXMALXXSAGXX*IXRSX |
| | | TTTTAACNNTAAAAGNNGNTNGGNCCTTNAGCATGAAATTTCTTTNT | | LTXKXXXXXSMKFLXCXXXLA*XA |
| | | TGTNCGGCTTAANGAGCNCTCATTNCATTTCATNACAAAGCNNTGAC | | LIXFHXKAXTXIFXSXXHXXLVXR |
| | | TANNATTTTTCACANNNTTCTTGTACNCCGCTTATATTAGNTNCTNT | | LY*XLXXWXFIXCXWXQPKTXHYV |
| | | TNNAATGGTTNTGTNACAACCCAAAACACNGCATTATGTNNACTGCC | | XCRHXSSLLXPXXERTPFPXXXWG |
| | | GTCATNGGTCATNCCCTTNNTNGAAAGGACTCCTTTTCCANTTNNTN | | MRCXGFLNFPPGXPXXPTLVFPFF |
| | | ATTGGCCGATGCGATTCTGAACTTTCCCCCNGGNNAACCAAGNGANC | | CG |
| | | CTACCCTAGTNTTTCCTTNC | | |
| prey84417 | 1086 | NNNNNNNNNNNNNNNNNNNNNNNNNNNNNNNNNNNNNNNNNNNNNNN | 1087 | XXXXXXXXXXXXXXXXXXXXXXXX |
| | | NNNNNNNNNNNNNNNNNNNNNNNNNNNNNNNNNNNNbINNNNNNNNN | | XXXXXXXXXXXXXXXXXXXXXXXX |
| | | NNNNNNNNNNGAGCCACTGCACCTGGTCAGTCACTTAGCTTATAACA | | SHCTWSVT*LITYIWLFGKYF*ID |
| | | TATGAAAATATTTTTAGATAGATGATATGTGTCATAATAGCTTATAA | | DMCHNSL*DIL*VLNRSFYRCNGE |
| | | GACAAAATAGGTCATTTTATAGGTGCAATGGGGAAAATACTCGATTT | | NTGF*SPKTLVRLQLSSYYLYKAP |
| | | TGAAGGTTAGGCTCCAGCTGTCCACTTACTATCTGTATAAGTGACCT | | HVSFLRFLNHP |
| | | CATGTAGATTTCTGAACCATCCC | | |
| prey84425 | 1088 | GTTACATTATGGCAGAAAAAAAGTGCAACTGACATACTACAAAGAGA | 1089 | TGQATLHYGRKKVQLTYYKEIF*V |
| | | AAAAAAGTTTGGATCTTTTGGATTTCTTTTTTTTTTTTNGGGCTTTA | | *KKFGSFGFLFFFXGFRGXKXXXX |
| | | GCNNNAATTNNAAATACCCCCCCNAAAANGGGNGGNTTTTTTTTTTT | | XIPPXKXXXFFFFXGPPQKXRXXV |
| | | TTAAAAANNNCGGGNNNGGGTTTTTNCCNTNNNCCNCCTTTCCNGGT | | FXXXXLSXFXXXPXPKGXPXXXPQ |
| | | TTTCCCAAAGGGCNNCCCCTTNNTTNNCCCCCAAGGGGNCCCCNTTG | | GXPXGXTXKKKXXXXGXXGNPSPF |
| | | GNNAANCGGNNNNAANGGGGGNNNAGGGGAACCCTTCCCCTTTTNGT | | XXXKKRAPPXNPXFXGXIXFXFXA |
| | | TNGCCCCCCCNNNAAACCCCNNTTTTNNNGGANAAATTTNTTTTTTN | | PX |
| | | TTTATTT | | |
| prey84428 | 1090 | CTATAAGAGAGGAGAACCTATTACCCAAGGGTGGGTATATAGGTTAA | 1091 | ACGTYYKRGEPITQGWVYRLIAWL |
| | | AATGCTACAGAGAAGGTCCTAGGCCAGCTTCTCCACACTGGCTCAAT | | *MLQRRS*ASFSTLAQF*YISALV |
| | | TGCCTTAGTTTAATGCCAAATTATTCCTGCTTTTTTCTCCATCACTC | | *CQIIPAFFSITQASLHSLISL*C |
| | | AGCTCTCATTTCTCTTTAATGTTATTAACACTAAACTGCAATGCTGA | | Y*H*TAMLIYF*KGYFSPMTKQLM |
| | | TATTTATTTCTCTCCTATGACCAAACAACTTATGTTTGTATAAACAC | | FV*TLWKPTNISHLALKKYYRVWK |
| | | TTTGATATCTCATTTGGCCCTCAAAAAATACTACAGGGTTTGGAAAC | | LF*LRI*ERNNQMEYVKPVWVLIR |
| | | TATTTAAGAGAGAAATAACCAAATGGAATATGTGAAACCTGTTTGCG | | KKETIKRHF |
| | | TGTTGAAGAAACCATAAAAAGACATTTCTG | | |
| prey84429 | 1092 | TACATTTTNGNANAIATGTGGATATTATACAATGNTATAATGTATAT | 1093 | GLTIYTFXXNVDIIQXYNVYLF*I |
| | | TAAATATTATGTATGATTTTTTTAACTTGCTTTCTACAATTTACATC | | KNIMYDFFNLLSTIYIIHCEHCYI |
| | | ATTGTTATATATAAATAAATATTCATCTATAACATCATTGTTAATGC | | *INIHL*HHC*CIVFY*RSTTTYL |
| | | ATAGAAGTACCATAACTTATCTACTCAACCTCTTTTTTTTTTTTTNN | | LNLFFFFXKXGXXFSPGGXKXXKK |
| | | AAACTTCCCCCGGGGGGGNAAAANGGGNCAAAAAAANNGTTTATTNC | | XVYXPPPXXGXXXPPXXPXRGXGE |
| | | CCCCCNNAGNNNNCCCCCCNCCCNNCCCCNCCGGGGNNNNGGGGAAC | | PGAPXPXXXGXFFFFFLXKKXXFS |
| | | CCGGGCNNGCCNGGGGTNTTTTTTTTTTTTTTTTTTGNAAAAAAANG | | |
| | | ANATT | | |
| prey84430 | 1094 | AACCAATTTCACCTCTTTGAAATTTTAAAGGCATATTTTAAACTTTT | 1095 | PTTPTTNFTSLKFYKGIF*T*KVI |
| | | GTCAACTACCTTACCTTTCTAACTTCACATATAAATTTGTTAACAAT | | CQLPYLSNFTYKFVNNKTISQIKV |
| | | AACAGATCAAAGTTGTAACCCACAGAAATCAAATACAGAGTGCAACA | | VTHRNQIQSATAQDKMLLFQLVQI |
| | | GCACAGTCTCTTCCAGCTTGTGCAGATCCATCAAATCTACTATTCAA | | HQIYYSTSYKST*YPYHIFPSCS* |
| | | CAAGCTACAATACCCATATCACATTTTCCCATCTTGTTCATAAGGAC | | GQPFVKLNIPLKSKDSIPLTNNLF |
| | | AGCCTTTTGTTAACGTTGAAATCAAAAGACAGCATTCCCCTAACTAA | | KLFRIYL*RKTRLAQNDLF*SLLS |
| | | TAACCTATTTAAACTTTTTAAAGGAAAACAAGATTAGCACAGATGAC | | PSGGLRTTAIPNFTLVRVSYVP |
| | | TTATTTTAGTCTCTCCGGGGGACTTAGGACCACTGCAAATCCCTAAA | | |
| | | CTTCACTCTTGTAACAGTTTC | | |
| prey84431 | 1096 | GTATCTGGGATGGATCTGAAGTTCTGAGGCGAATCCGAGGACCACTG | 1097 | KSEFQYLGWI*SSEANPRTTACSG |
| | | TATTAAATATTTTCTCTTTACATAAAATTTATAATATCCAAAATTTT | | HPFLTEGVYRSFIC*NYVDYCIQH |
| | | AAGCATCCTTTCCTTACTGAAGGGGTTTATAGGTCTTTCATTTGCTA | | LAMPFFSLQGKGCKGAIGI*IC*C |
| | | GAAATTATGCATCCAGCACTTAGCTATGCCTTTCTTTAGCCTCCAGG | | MIVI*IT*KVISEPR |
| | | GTAAAGGTTGCATGGAATTTAAATTTGTTAGTGTATGATTGTGATTT | | |
| | | GATCACTTAAAAAGTCATATCTGAGCCCAGGCC | | |
| prey84432 | 1098 | TCTCTAGACCTAATGTTTTATGTAGGGACCTCTAGCCACTTCAGGGA | 1099 | ILIPFL*T*CFM*GPLATSGKEET |
| | | GAAGTACTATTTATTGAGTCACCTAATCTGTTTTCGATGCCGTATGA | | KYYLLSHLMCFRCRMRCFADFHNP |
| | | GGTGTTCCACAATCCTATGAATAAATACTAATATCCAGATTTTTTTT | | MNKY*YPDFFLS*EAKVQND*VT* |
| | | TGTCATGATTCAGAATGATTAAGTGACATAATGAGCTAGTAATTGTA | | *ASNCKSWNSNPERSACKTQVSSL |
| | | AGAGCTGGAATTACGCTCCGCCTGCAACCCAAGTGTCTTCACTTTTT | | FPVQ*LCYVFSP |
| | | CCAGTCCAAAAACTTTTAGCCCGAC | | |
| prey84433 | 1100 | GTCTCTTTTAGCTNTTTTGTAAATTTGAGATACTTTTCATTAATTAT | 1101 | CHHNWSLLAXL*FEITFH*LLLFC |
| | | GAAGTACTATTTATTGAGTCACCTAATGTGTTTTCGATGCCGTATGA | | RVTKNKNLFLLSXCNTILLKLPFP |
| | | GGTGCTGCCTTTTCCCATCTCCCTGCTTCACTCTCTNGGAACCCCTN | | ISLLQSLGTPXVLFAYXXPFFQVP |
| | | TTGTACTCTNANCCCTTTTTTTCAGGTTCCTCANATGAGTGAGATAA | | XMSEIIXSXXXLGESXWLN*LKKG |
| | | TAANATCAAAANANTTTCTCTTTNTGGCTTAATTAACTTAAAAAGGG | | X*GSSLPFY*KIFSFASLKGFXWA |
| | | ANGCTPAGGGTCATCCCTTAAAAAATTTTTTCCTTTTAAAAGCTTAA | | NLGNFX*PLXLGXX |
| | | AAGGATTTCNCTGGGCAAATTTGCCTAACCCCTTTNTCTGGGGGNGN | | |
| | | AN | | |
| prey84439 | 1102 | NNNNNNNNNNNNNNNNNNNNNNNNNNNNNNNNNNNNNNNNNNNNNNN | 1103 | GXXXXXXXXXXXXXXXXXXXXXXX |
| | | NNNNNNNNNNNNNNNNNNNNNNNNNNNNNNNNNNNNNNNNNNNNNNN | | XXXXXXXXXXXXXXXXXVPLYPAN |
| | | NNNNATACCCAGCTATCTTTTNNNNNNNNNNNNNNNNNNNNNNNNNN | | LXXXXXXXXXXXXXXXXXXXXXXX |
| | | NNNNNNNNNNNNNNNNNNNNNNNNNNNNNNNNNNNNNNNNNNNNNNN | | XXXXXXXXXXXXAHTARLIFDLRW |
| | | NNNNNNNNNNNNNACACACAGCCAGATTGATTTTTGATTTACGATGG | | VFGGRNPAVRSICTHIPS*WWTCL |
| | | GTTTTTGGGGGGCGGAAAAGGAGCATCTGTACTCATATTCCCTCATC | | YS*FC*VLLS |
| | | ATGGTGGACTTGTTTATATTCGGAGTTTTGCTTTCTGT | | |
| prey94442 | 1104 | NNNNNNNNNNNNNNNNNNNNNNNNNNNNNNNNNNNNNNNNNNNNNNN | 1105 | XXXXXXXXXXXXXXXXXXXXXXXX |
| | | NNNNNNNNNNNNNNNNNNNNNNNNNNNNNNNNNNNNNNNNNNNNNNN | | XXXXXXXXXXXXXXXXXXXXXXXX |
| | | NNNNNNNNNNNNNNNNNNNNNNNNNNNNNNNNNNNNGAAAAGATATA | | XXXXXXXKKDIEGTNDYLVTNYFS |
| | | GAAAGGACTTGGTTACCAACTATTTTAGTTGTATTTTGTATAGTACT | | CILYSTIL*IYVHI*HRHEWKAGI |
| | | ATATTATAAACATCTAACACAGGCATGAGTGGAAGGCAGGCATACAA | | QHPLKHFY*WSIQHITK*LYG*LG |
| | | ACATCCTCTTAPACATGGAGTATTCAGCACATCACAAAGTAGCTTTA | | F*IFLNY*VTLILSFILX |
| | | TGGATAGCTTGGCTTCTGACTATTAAGTAACATTAATAATTTCCTTT | | |
| | | ATTCTATGNGC | | |
| prey84447 | 1106 | AGGAAGGAAGGAGAAGGCAGGCAGGAAATCAATATGACAGCATAGCA | 1107 | EGGEKEGRRRQAGNQYDSIALELR |
| | | TACCTTCTAGAAGCAGACTCTATGAATTCAAATCCTGGGTCTACCAT | | THLLEADSMNSNPGSTIYKLYFPL |
| | | GTACTTTCCTCTGTAAAATATGAATCTACAGTACCTACTTCTCACAG | | *NMNLQYLLLTDCYKDEMC*YR*S |
| | | ATTGTGAGATGTGTTAATATAGATAGTAAGCACTCAGTGAATATGAG | | KHSVNMRH*PEPNKVPSSNIIIEC |
| | | GCATTAACACAAGGTGCCATCATCAAATATAATAATTGAGTGCATAA | | ITFFSLQSKFTYSTSFGS |
| | | CTTTTTTCAGTTCATATAGCATTTCTTTTGGATCCC | | |
| prey29055 | 1108 | CATCAACATGTTCCTCTTCTCCCACAGCCCCAGCAACAGGCTGCTCT | 1109 | LIHFINMFLFSHSPSNRLLYPRQE |
| | | ACGAGGTGGTCCAGGCCACGCTGGTGGTCCTGGCCTTGGCCATGGTG | | VVQATLVVLALADIVPILLLGTPL |
| | | CCCATCCGCACACCCCTGCACCTGCTGCACCGCCACCGCCGCCGCCT | | HLLHRHRRRLRRRPADRQEENKAG |
| | | GCGGAGGAGGCCACAGGAGGAAAACAAGGCCGGGTTGCTGGACCTGC | | LLDLPDASVNGWSSDEEKAGGLDD |
| | | CTGACGCATCTGTGAATTCCGATGAGGAAAAGGCAGGGGGCCTGGAT | | EEEAELVPSEVLMHQAIHTIEFCLGCVSN |
| | | GATGAAGAGGAGGCCGAGCTCGAGGTGCTCATGCACCAGGCCATCCA | | |
| | | CACCATCGAGTTCTGCCTGGGCTGCGT | | |
| prey84460 | 1110 | NNNNNNNNNNNNNNNNNNNNNNNNNNNNNNNNNNNNNNNNNNNNNNN | 1111 | GXXXXXXXXXXXXXXXXXXXXXXX |
| | | NNNNNNNNNNNNNNNNNNNNNNNNNNNNNNNNNNNNNNNNNNNNNNN | | XXXXXXXXXXXXXXXXXVPLYPAN |
| | | ACCAGCTAATCTTTTNNNNNNNNNNNNNbINNNNNNNNNNNNNNNNN | | LXXXXXXXXXXXXXXXXXXXXXXX |
| | | NNNNNNNNNNNNNNNNNNNNNNNNNNNNNNNNNNNNNNNNNNNNNNN | | XXXXXXXXXXXXAHTARLIFDLRW |
| | | NNNNCACAGCCAGATTGATTTTTGATTTACGATGGGTTTTTGGGGGG | | VFGGRNPAVRSICTHIPS*WWTCL |
| | | CGGAAAGCATCTGTACTCATATTCCCTCATGATGGTGGACTTGTTTA | | YS*FC*VLLSVLSGSIRHLKV*NC |
| | | TATTCGTTTTGCTTTCTCTGCTTTCAGGCTCTATCCGGCACCTTAAG | | PVF*VKPXXXXXXXXXXXXXXXXX |
| | | GTTTAGATTGAGTCAAACCNNNNNNNNNNNNNNNNNNNNNNNNNNNN | | XXXXXXXXXXXXXXXXXXXXXXXX |
| | | NNNNNNNNNNNNNNNNNNNNNNNNNNNNNNNNNNNNNNNNNNNNNNN | | XXXXXXXXXXTA |
| | | NNNNNNNNNNNNNNNNNNNNNNNNNNNNNNNNNNNNNNNNNNNNNNN | | |
| | | NNNNNNACCG | | |
| prey84461 | 1112 | ATAAGGGAATAATATGGTACTTCTCACCTTACTGGGCAAATTAAATA | 1113 | YII*YKGIIWYFSPYWAN*IPVNN |
| | | CAAGGATTTCCTTGTAATACAAATAACTTTTTTTTAAAATATAGAAT | | LRISL*YK*SFFLKYRSSELDLFS |
| | | AAGACCTGTTTAGCTCAGATAGTGGGCTAAACTACCATAAAATTCTC | | SE*WAKLPANSLYILNGNGSKTPE |
| | | TCTATGGTAATGGGTCAAAAACTCCAGAAAATCATCAGTTGATAAAC | | NHQLITHLQISAWVGGDSQVPMII |
| | | ACACCTACAGAGGGTAGGAGGGGATAGCCAACTCCCCATGATAATTT | | *PQ*IKLGNTHICYSDTALDL*NS |
| | | GACCTCAGTAAATTAATACACATATTTGCTATTCTGATACTGCATTA | | T**AFIKLDLSVYI*LRQG*GKE* |
| | | GACTTATAAATTCCATCTAAAATACTGGACCTCTCTGTATATATCTA | | LKKLAYNS*VSSGLFYPARNHHQR |
| | | GCTTAGACAGGGATAGGGAAAGAGAAACTAGCTTACAATAGCTAGGT | | EAEPFSCLSPLPLFE*SCLGLAAD |
| | | TTCGTCAGGCTTATTCTATCCAGCCAGCCACACAGAAGCTGAGCCAT | | QSLFVRSRKRL*K*KGGGGNQS*K |
| | | TCAGCTGTCTGTCTCCTCTCCCTCTCTTTCAACTAGGCCTTGCTGCA | | KLWGG |
| | | CACCAAAGCCTGTTTGTTAGGAGCAGGAAGAGACTTTAGGGGCGTGG | | |
| | | GGCAAACCAAAGTTAAAAAAAAATTGTGGGCGCCCC | | |
| prey93104 | 1114 | TTCCAAGGTACAGCTCAAATACTACCCCGTTTTATATAACTTTCCTT | 1115 | NSTHYSKVQLKYYPVL*NFP*FSQ |
| | | AGTTAAACGTCTTATATCCTGCCTCTGAAGTTCCCTTAACGTCATAG | | LNVLYPASEVPLTS*ESLLSYLID |
| | | GAGTCTTATCTCATTGACTAGATTACAAGCCTTGACTAACATTTTTC | | AITSLD*HPSSL*AHGSFTLLLIG |
| | | ATCCTTGTGATCTTTTACCTTGCTCCTAATACGTACTCAACTGTTTG | | TQQMFA**MYFVRLEDLIYILTSL |
| | | CTTAATAAATGTGACTCGAAGACTTAATATATATACTGACATCATTG | | LS*KIHLWKCIHFQFFIWGCRLSN |
| | | CTCTCTTAAAAAATACACCATTTCCAATTTTTCATTTGGCGATGCAG | | TSLPPGTSSLPRDRVAEDTAIIII |
| | | GCTTTCCAATACATCCGAACCAGCAGTCTACCCAGAGATAGAGTTGC | | SRDKGEESLRE*SPKVLRNPGFTA |
| | | TGAGGACACTGCAATTATCAAGACAAAGGTGAAGAAAGTCTAAGGGA | | FSP |
| | | ATAAAGCCCAAAGGTACTGAGAAACTTTCTCTCCC | | |
| prey84464 | 1116 | ATAGCAAATTATTTAGATATCTTGATMATTTATATATAACCATAACC | 1117 | WGTQIIAII*IS**FIYNHNHIGD |
| | | GAGATAGTAGAAATATATAGTATAAGGTCTTTCATATTCAGCCATGC | | RR**KYIV*GLSYSAHINTTHTHT |
| | | ACACACACACACACACACAGACACACACGTCCACATACATGTTCATA | | HTDTHVHIHVHKYLGIQIVCILSC |
| | | AAAGATAGTTTGCATCCTTTCCTGCTCTCACCAGCCTGGGTCAAAAA | | SHQPGSKMLSSISFMIXAKXPMFL |
| | | TGTTTTCATGATAAGNGCCAACNCCCCAATGTTTCTGATCATTTTGC | | IILPCH*LKYTPKFFSSDSFVXKS |
| | | CTTGTATACTCCCAAATTCTTCAGCAGTGACTCCTTTGTGGNGAAAT | | CGCIWXQITFGRQLC*ETXL |
| | | CCTGTNGCAAATTACCTTTGGTAGGCAATTGTGTTGAGAGACACANC | | |
| | | TCT | | |
| prey84466 | 1118 | TTTACTANAGGGCCTTACTATTGTTGCATTCAGNTAATAATTGTNCA | 1119 | RNLLHFTXGPYYCCIQXIIVLFGG |
| | | GCTTCCANATGAGGAATCTGCTGCATTTTACTAAAGNGCCTTGCTAA | | *ASX*GICCILLKXLAKMAXVVAK |
| | | GTGGCAAAATGACGTATATGCTCTCAATTATATATACCTGGCACACT | | *RICSQLYIPGTLELPC*EYNKVA |
| | | TAGGAATATAATAAGGTTGCCATTAATAAAAATTTATAACCTTTGAA | | INKNL*PLK*N*NLILFTPCAHFP |
| | | GTAATTTTATTTACTCCATGTGCACATTTTCCTCGTTTAGTCAGNTA | | RLVXYLDSLRRACI**CSHVXHKK |
| | | CTTAGAGCATGTATTTGATAATGTTCTCACGTTNNACATAAAAAAAA | | KVNXXSXXXIXALXTQSXT*NELM |
| | | AGTAGNNTATANATTTGNGCTTTANTAACTCAGTCNCANACATAGAA | | AFTWWXKYLTNKIVGIXXX |
| | | TGAATATGGTGGGNAAAGTATCTTACAAATAAGATAGTAGGCATTTN | | |
| | | GNANNG | | |
| prey84470 | 1120 | GGTTTGCAAGTTCTCTGCATTGTATAGTAACAAATTTTCTTCTTTCT | 1121 | GQAVLGLQVLCIV**QIFFFLYSV |
| | | TGGAGGCAAGTCAAGAACGGAGGGTTTAACTCCATCTCCTGTGAGGG | | LWRQVKNGGFNSISCEGEAYVNLL |
| | | TTATTATTTGGAATTCTTCTTTAAGACATATTTGCCACTTCCACTTC | | FGILL*DIFATSTSLPFI*LFIYI |
| | | TAATTGTTTATTTATATCAATGTGGACTCCTATATGTACACATTTAT | | NVDSYMYTFIF*P*TLLEFYQFFY |
| | | TTTTTATTAGAATTTTACCAGTTTTTCTATGAATGTCCTTTTCTGTT | | ECPFLFLVPIQDTTLHLIVMFS*S |
| | | CCTGACACCACATTACATTTAATTGTAATGTTTTCTTAATCTCCCTC | | FSGL*RXPHSLFIFS*P*QX*GXT |
| | | TGGNCTCATTCTCTTCCTATTTTTTCATAACCTTAACAGNNTTGAGG | | XPI |
| | | ANGAAAT | | |
| prey84471 | 1122 | GGAGGCAAAGGAACAAGAAATGTGCCTTGGGTGGACAGGGAACATTC | 1123 | YKDLKEAKEQEMCLGWTGNILGGI |
| | | AGTGCAGAAATCATTAAGGCAGAAAGATCCCTACTACATTCTAAGAA | | TSAEIIKAERSLLHSKNRKQSRVI |
| | | AGAGTGATAGAAGCAGAGTACAGGAGGGTAAAAGTAGGAAATGAAGT | | EAEYRRVKVGNEVREVRGIQIGHG |
| | | CGAATTCAAATTGGGCATGGACTTACAGGCCATTGTAAGGACTTTGG | | LTGHCKDFGFF*GRWEAIVK*KNS |
| | | CTATGGGAAGCCATCGTTAAGTAAAAAAATAGTGAAATTCTACTTAT | | EILLIYLKNHIDF*FDTRLLRSKG |
| | | ATAATTGACTTCTAGTTTGACACTAGATTATTGAGGAGCAAGGGGGA | | X |
| | | NC | | |
| prey84491 | 1124 | TTCAGTTTGATCTGAGTCTACCCAGGATCAGTTGGATTTATACAGCA | 1125 | WVYLGSV*SESTQDQLDLYSIWA* |
| | | AATTGCTGCTATTTTATGTGTAGTATCAAATTAATCCCTTAGACTGA | | *IAAILCVVSN*SLRLKFLLINF* |
| | | AAACTTTTAATTGGTTTTAGTCAGTGTATTAACATGGAAATAAAAGA | | LVLVSVLTWK*KNTK*F*LFDFYH |
| | | ATAATTATTTGATTTTTATCACTAATGATTGAAAGACCAATGAGAAA | | **LKDQ*ETSDTLFIFQWNKQELL |
| | | CTTCATTTTCCAGTGGAATAAACAAGAATTACTTAGGTTGAGAGAAA | | RLRENLYFCISIFLL*QN*INEIL |
| | | ACCTTCTATATTTCTGCTTTAACAAAATTAAATAAATGAAATTTTAA | | KLDIGTCISMCVGTRISYCFYL*K |
| | | AGTTGCTTGCATATCTATGTGTGTGGGCACACGTATATCCTATTGTT | | SFIGQVXXXXXXXXXXXXXXXXXX |
| | | TTTACTTGTTATTGGCCAAGTNNNNNNNNNNNNNNNNNNNNNNNNNN | | XXXXXXXXXXXXXXXXSPLKQLF* |
| | | NNNNNNNNNNNNNNNNNNNNNNNNNNNNNNNNNNNNNNNNNNNNNNN | | IGWAWWSVPVVLLLRSGGCAAKGLHGGT |
| | | NNNNNNNNNNNNNNNNNTGAAACAATTGTTTTMXATTGGCTGGGCAT | | |
| | | GGTGGTCCGTGCCTGTAGTCCAAGCGGAGGCTGTGCCGCAAAAGGCC | | |
| | | TTCATGGTGGCACC | | |
| prey84492 | 1126 | ACGTTACATTATGGCAGAAAAAAAGTGCAACTGACATACTACAAAGA | 1127 | GQATLHYGRKKVQLTYYKFAIF*V |
| | | GATTAAAAAAAGTTTGGATCTTTTGGATTTCTTTTTTTTTTTTGGGG | | *KKFGSFGFLFFFWGFKALKNP*L |
| | | CTTTAGGCCNTAATTAAAATTACCCCCCCCAATTAAGAGGTTTTTTT | | KLPPPIKKVFFFFFGQXPKTPXGV |
| | | TTTTTTTTTCCAAAAACCCCGNTGGGGGTTTTTTCCTTTTCCCCCCN | | FSFSPXSQFCGVPFPKEPPLINPP |
| | | TTCCCAATTTTGCTCCCAAAGGAACCCCCCTTAATTAACCCCCCAGC | | GGHLLKPPKKNRAQGGKGETLALL |
| | | GGGGCATTTGTTAAAACAAACCGGGCCCAAGGGGGGAAAGGGGAAAC | | VWKNPPQKKTHFFQKLFFLKGAPX |
| | | CCTGGCCCTTTTGGTTTGGAAACAAAAAAAAACCCATTTTTTTCAAA | | |
| | | CTTTTTTTCCTTPAAGGAGCCCCT | | |
| prey72968 | 1128 | TCTCCTGGGATTGGAACGACCCCTATGAGGTGGCCCACAGCATGGCC | 1129 | RSCKVSWDWKDPYEVGHSMAFRVH |
| | | ATTTATTCTATAAGAACGGGCAGCCTTTCCCTGCACATCGGCCTGTG | | LFYKNGQPFPAHRPVGLRVHISHV |
| | | GGACCATCTCTCATGTCGAGCTAGCAGTGGAAATTCCAGTGACCCAG | | ELAVEIPVTQEVLQEPNSNVVKVA |
| | | GAAAGTCCTAATTCCAACGTAGTAAAAGTGGCCTTCACTGTGCGCAA | | FTVRKACRYEITVKLGGLNVAYSP |
| | | GGCTGGGCGTTATTGAAGCTTGGTGGATTAATGTGGCATATAGTCCC | | YYKIFQPGMVVPSKTKIVCHFSTL |
| | | TACTACAAAAATTTTTCGGTGGTTCCTTCTAAGACCAAAATTGTCTG | | VLTCGQP |
| | | CCACTTTTCTACTCTTGTATTCAGCCG | | |
| prey84494 | 1130 | ATCTGATCAGGGATCCAGGGACCCACAAGAGATAGAGTCAGGTAAGT | 1131 | DWKRSDQGSRDPQEIESGKFKKEE |
| | | TCAAGCACAAGGAAGAAAAATCTTGTGTTGGCTCTGAGGTAIACTAA | | AQGRKILCWL*GN*PNTPWNKFSI |
| | | CCAATACAAATTCAGCATTTTAATTGATTGCAAGATTGCACCAGCTA | | LIDCKIAPANTVCIANPSAQHLK* |
| | | TACTGTGTGTCTAGTGCTCAGCACCTGAAGTAGTGCCAGACTTGGGG | | CQTWGASKNQQKI*NITLYFAWKT |
| | | AGCGTCTAAAAATCCTGAAATATAACTTTATACTTTGCCTGGAAACA | | P |
| | | CCAC | | |
| prey84496 | 1132 | TACATTNTGNCACAAAAGTCCNTCTGACATACTACAAAGAGATNNAG | 1133 | GHATLHXXTKKVXLTYYKEXX*VX |
| | | AAANAGTTTGGATCTTTTGGATAATCTTTTTTTTTTTTGGGNCNGCA | | EXVWIFWIIFFFFGXACXLSNRHX |
| | | TGACCGACACANGGGAACAGCCTCCNCATTTGNNCTGGNTTANATTN | | GTASXFXLXXIXFYXXXPRKXVDE |
| | | TTTTTGCCCAAGAAAANCTGTGGACGAAANAGGGCNTTCCTCACTCC | | XGXSSLLLLXGRHDSERDAPSASP |
| | | TTTTACTCATGACAGTGAAAGAGATGCTCCNTCTGCTTCNCCCCCNN | | PXXRXGPXXXEHQLTXXLRGSXAP |
| | | GGNCTCGCTGNGGTANGAACATCAGCTTACTNTGNNNCTNAGGGGAA | | XGXVSSGSPQXXPFFXNSXLX |
| | | GCANNGCCCCTTTNGGNCAGGCTCNCCNCAGGNTNCGCCTTTTTTTA | | |
| | | NAAAAACAGTNCCCTTCNTG | | |
| prey100887 | 1134 | CAGCCTTCTCTTAATAAAATTAAGTTGTATTGTTTCATCTGNNNNNN | 1135 | SSAHQPCLNKIKLYCFIXXXXXXX |
| | | NNNNNNNNNNNNNNNNNNNNNNNNNNNNNNNNNNNNNNNNNTTTTTN | | XXXXXXXXXXXXXFXGPGGPXXXX |
| | | GGGNCCCNTNGNNNTCNNNNCNNNNGGGNCCCCCCCCCNNTTTNCCT | | XXXXPPPXXLXGGVXKNXGGGFXX |
| | | TTTNGGGGGGTNTNGGGGGGGGNTTTNNNNNCGCCGGGGGGGGGAAA | | GGGGKXPFFXXXXCKXPPXFFFL |
| | | NCCCCTTTTTTTTTAAAANCCCCCGNNNTTTTTTTTTTTG | | |
| prey100896 | 1136 | GTGCCAGGAGAGTGGCAGTGCCATGCTGCTGGGAGCTGCCCCGGAGA | 1137 | GRLVCARRVAVPCCWELPRRAAQD |
| | | GGGGGCCGCCCCTCGTCCTCTCTCGTCCCCGACGGGTCGGGCAGGTC | | PGGAAPRPLSSPRGRAGRKIKLYS |
| | | AAACTTTATTCCCCTCTGTGACTTCCTCTGTGTGTGTGCATGCGGAA | | PL*LPLCVCMGNRLPRDGCCIASG |
| | | CCGGATGGATGCTGCATTGCTTCAGGAGGCAAGCTTCATCCTCTTGG | | GKLHPLGSLKVLLGVLSRATVAAC |
| | | GTCCTTAGGGGGTCCTTTCGAGGGCTACTGTATAAGCGTGTTCACGC | | SRVPEAGRVVSRHRRSEYGKLLTE |
| | | GTGCCTGAAAGCGGGAAGGGTGGTCAGCAGGCACAGGAGAAGCGAAT | | AEIWWLY*FVVA |
| | | ATGGTAAGTTGTTGACAGAGGCAGAGATTTGGTGGTTGTATTAACCA | | |
| | | GTTGTTGCTCG | | |
| prey100897 | 1138 | NCATTTCCCAATGACCTAATGATGTGTGAGNATCTTTTTGCGTGCTP | 1139 | XFPNDLMMCEXLFAC*TCHPLSPL |
| | | AAACNTGCCATCCTCTGAGTCCTCTTNCGNGAAATGTGCTTNATGGT | | XXNVLXGLLSYXSI*GLWLFLXXX |
| | | CTTTTGTCNTATTNTTCTATTTAGGGCCTTTGGCTCTTTTTACNANT | | XGRGFFYTXLXXXFPFVXIXGWXL |
| | | GNGGTTNGGGAGAGGTTTTTTTTATACAGNCCTTANATNANANAATT | | EXFLRIIINFVPGEKXSSQFSXPF |
| | | TCCTTTTGTTAANATATGNGGGTGGNTGTTGGAANTTTTTTTAAGAA | | GLAXEXLXXXPWXFRGXPCXRNFA |
| | | TTAACTTTGTACCAGGGGAAAAANTAAGTTCNCAATTTTCCNACCCC | | XXXXF*DLXXLGGXXXKXXXLXNX |
| | | TTTGGATTGGCGNAGGAAANTTTANTTTNANCTCCCTGGNCCTTTAG | | YXXFXXXXPXFFXXXXXLXL |
| | | AGGGNTNCCATGTNAGACAAATTTTGCANNANTTTTNNNGTTTTAAG | | |
| | | ATTTANCNNNNTTGGGGGGNNCTNGCTNGAAANNCNNNNNNCTTTNA | | |
| | | AACATNTATTTNNTTTTTNAAANCNAANACCCCTTNTTTTTCTTNTN | | |
| | | GTNGGNGNTTTTGGNACTNG | | |
| prey100898 | 1140 | CTTTCCTTTGGGGTCTAGAGTGAAAGCTAATTTGCGGGTAGATGAGA | 1141 | LSFGV*SES*FAGR*DCRRNGCPW |
| | | TTGCAGAAGGAATGGATGTCCATGGCTGNGAACACTGCACACTGCAC | | LXTLHTAHPSPVLTLCSYHSLAAC |
| | | ATCCATCTCCAGTGCTCACACTGTGCAGCTACCACTCCCTGGCTGCG | | HAVGLQICPHKFLRKSLHEHHLAI |
| | | TGCCATGCTGTCGGGTTGCAGATTTGCCCACATAATTCCTCAGGAAG | | FCTDQTRDLNVFQHKRITSEWWGX |
| | | AGTTTGCATGAGCATCACCTCGCAATATTCTGTACTGACCAACAAAG | | YAYXQR*W*SSKTKLVFLQLEVSQ |
| | | GGATTTGAACGTTTTTCAACACAAAAGGATAACTTCCGAGTGGTGGG | | GALRIVVXKXIPGLIFFFKPE*PP |
| | | GCNGGTACGCATACTANCAAAAGGTAATGGTGATCTAGCAAAACAAA | | XRIX |
| | | ATTGGTGTTCTTGCAGTTAGAAGTGACCCAGGGAGCACTTCGTATTG | | |
| | | TAGTATTNAAAANAATCCCTGGTTTAATTTTCTTTTTTAAGCCGGAC | | |
| | | TAACCCCCTCNCAGGATTTTNT | | |
| prey100899 | 1142 | TCCANNNNNNNNNNNNNNNNNNNNNNNNNNNNNNNNNNNNNNNNNNN | 1143 | SXXXXXXXXXXXXXXXXXNGKGKX |
| | | NNAAGNAACGGAAAGGAAAAANAANAAAACCATTAGAATTTTTANTN | | XKPLEFLXSXRGXXXXSTPFQXLX |
| | | TCCNTCCGGGGNANTNTAANNNATTCCACCCCNTTCCAAAANCTTTN | | GXVXXXXKFXXXXXXXXXFXXFXX |
| | | NGGNANNGTTNAGNAAANTNGAAAATTTTNNTNCNNNGNANGTNANN | | KXPXXXXFXKCXXXXGXGXVXXXX |
| | | AGTNTNNNTTTTNTTTNTTTNTGCNAAAAAANCCNTNAANCTNCCNC | | XRXXLGXFXX*XPXRXKSXLNXIF |
| | | TTTAGNAAATGTTNTANNGANNCTGGTNGGGGCTTNGTNCNTNANCN | | GFXLXXTSQXXFGTKVAXX |
| | | GGANTNTAGANTAAANCTNGGTTNGTTTNTA1ANTAAANTCCTNCCC | | |
| | | GGTNNAAAAGTNGATTAATTGNATTTTTGGGTTTNATTTGGNANNAA | | |
| | | CNTCNCAANGNTANTTTGGACAAAAGTAGCTTNGTTNC | | |
| prey78825 | 1144 | CCCTAAGCGAATAGCACAACACAGCCAGCTGMATCAAACACCATCAG | 1145 | PKKIAQTQPAESNTISRITANMEN |
| | | TAGGATAACTGCAAACATGGAAAATGGAGAAAATGAAGGAACAATTA | | GANAGTIKIIAPSPVKSFKKAKNE |
| | | AAATTATTGCACCTTCACCAGTAAAAAGCTTTAAGAAAGCAAAGAAT | | NSPDTQRSKSLMHSWEENGPQSGL |
| | | GAAAATAGCCCTGATACCCAAAGAAGCAAATCCCTCATGCACTCGTG | | YNSPSDRTKSPKFPYTRRRNPSCG |
| | | GGAAGAAAATGGCCCCCAGAGTGGACTCTACAATTCTCCCAGTGATC | | SDNDSVQPVRRRKAHNSGEDSDLK |
| | | GCACTAAGTCGCCAAGTTCCCTTACACGCGTCGCCGAACCCCTCCTG | | QRRRSRSRCNTSSGSESENSNREH |
| | | TGGAAGTGACAATGATTCTGTACAGCCTGTGAGGAGGAAAGCCCATA | | RKKRNRIRQENDMVDSAPQWEAVL |
| | | ACAGTGGTGAAGATTCAGATCTTAAGCAAAGGAGGAGGTCACGTTCA | | RRQKEKNQADPNSRRSRHRSRSRS |
| | | CGCTGTAACACCAGCAGTGGTAGTGAATCAGAAAATTCTAATAGAGA | | PDIQAKEELWKHIQKELVDPSGLS |
| | | ACACCGCAAAAGAGAAIACAGAATACGGCAGGAGAATGATATGGTTG | | EEQLKEIPYTKIETQGDPIRITHS |
| | | ATTCAGCGCCTCAGTGGGAAGCTGTATTAAGGAGACAAGGAAAAAAC | | HSPKLY* |
| | | CAAGCCGACCCCAAACAGCAGGCGATCCAGACACAGATCTCGTTCGA | | |
| | | GAAGCCCCCGATATCCAAGCAAAAGAAGAGTTATGGAAGCACATTCA | | |
| | | AAAGAACTTGTGGATCCATCCGGATTGTCCGIGAACAATTAAAAGAG | | |
| | | ATTCCATACACTAAAATAGAGACACAAGGTGACCCAATCCGCATCAC | | |
| | | GCATTCTCATTCGCCAAAGCTTTATTAG | | |
| prey100901 | 1146 | GGACAGCGGTGTGGGGCGGACCGACGAGAGCACCCGTAATGACGAGA | 1147 | DSGVGRTDESTRNDESSEQENNGD |
| | | GCTCGGAGCAAAGAGAACAATGGCGACGACGCCACCGCATCCTCCAA | | DATASSNPLAGQRKLTCSQDTLGS |
| | | CCCGCTGGCGGGGCAGAGGAAGCTCACCTGCAGCCAGGACACCTTGG | | GDLPFSNESFISADCTDADYLGIP |
| | | GCAGCGGCGACCTGCCCTTCAGCAACGAGTCTTTCATTTCGGCCGAC | | VDECERFRELLELKCQVKSATPYG |
| | | TGCACGGACGCCGACTACCTGGGGATCCCGGTGGACGAGTGCGAGCG | | LYYPSGPLDAGKSDPESVDKELEL |
| | | CTTCCGCGAGCTCCTGGAGCTCAAGTGCCAGGTGAAGAGCGCCACCC | | LNEELRTCELECLSIVRAHKMQQL |
| | | CTTACGGCCTGTACTACCCTAGCGGCCCCCTGGACGCCGGCAAGAGT | | KEQYREYWMLHNSGFRNYNTSIDV |
| | | GACCCTGAGAGCGTGGACAAGGAGCTGGAGCTGCTGAACGAAGAGCT | | RRHELSDITELPEKRQGQLERLQH |
| | | GCGCACATGCGAGCTGGAGTGCCTGAGCATCGTGCGCGCCCACAAGA | | RRELPQHPAHPGDLPRQLLEESGG |
| | | TGCAGCAGCTCAAGGAGCAGTACCGCGAGTACTGGATGCTGCACAAC | | GHQLPEQRRGCGDHGSLRASLQES |
| | | AGCGGCTTCCGCAACTACAACACCAGCATCGACGTGCGCAGACACGA | | ALHHGRSRSGHPYL* |
| | | GCTCTCAGATATCACCGAGCTCCCGGAGAAACGACAAGGACAGCTCG | | |
| | | AGCGCCTACAACACAGGCGAGAGCTGCCGCAGCACCCCGCTCACCCT | | |
| | | GGAGATCTCCCCCGACAACTCCTTGAGGAGAGCGGCGGAGGGCATCA | | |
| | | GCTGCCCGAGCAGCGAGGGGCTGTGGGGACCACGGAAGCCTACGGGC | | |
| | | CAGCCTCCAAGAATCTGCTCTCCATCACGGAAGATCCCGAAGTGGGC | | |
| | | ACCCCTACCTATAG | | |
| prey56846 | 1148 | CACCGAGCCACCAACCCAGAAGCCCAAGAAGATTGTAAATGCCAAGA | 1149 | TEPPTQKPKKIVNAKKDVVNTKMF |
| | | AAGATGTTGTGAACACAAAGATGTTTGAGGAGCTCAAGAGCCGTCTG | | EELKSRLDTLAQEVALLKEQQALQ |
| | | GACACCCTGGCCCAGGAGGTGGCCCTGCTGAAGGAGCAGCAGGCCCT | | TVCLKGTKVHMKCFLAFTQTKTFH |
| | | GCAGACGGTCTGCCTGAAGGGGACCAAGGTGCACATGAAATGCTTTC | | EASEDCISRGGTLSTPQTGSENDA |
| | | TGGCCTTCACCCAGACGAAGACCTTCCACGAGGCCAGCGAGGACTGC | | LYEYLRQSVGNEAEIWLGLNDMAA |
| | | ATCTCGCGCGGGGGCACCCTGAGCACCCCTCAGACTGGCTCGGAGAA | | EGTWVDMTGARIAYKNWETEITAQ |
| | | CGACGCCCTGTATGAGTACCTGCGCCAGAGCGTGGGCAACGAGGCCG | | PDGGKTENCAVLSGAANGKWFDKR |
| | | AGATCTGGCTGGGCCTCAACGACATGGCGGCCGAGGGCACCTGGGTG | | CRDQLPYICQFGIV* |
| | | GACATGACCGGCGCCCGCATCGCCTACAAGAACTGGGAGACTGAGAT | | |
| | | CACCGCGCAACCCGATGGCGGCAAGACCGAGAACTGCGCGGTCCTGT | | |
| | | CAGGCGCGGCCPACGGCAAGTGGTTCGACAAGCGCTGCCGCGATCAG | | |
| | | CTGCCCTACATCTGCCAGTTCGGGATCGTGTAG | | |
| prey32308 | 1150 | ATGTACGAGCAACTCAAGGGCGAGTGGAACCGTAAAGCCCCAATCTT | 1151 | MYEQLKGEWNRKSPNLSKCGEELG |
| | | AGCAAGTGCGGGGAAGAGCTGGGTCGACTCAAGCTAGTTCTTCTGGA | | RLKLVLLELNFLPTTGTKLTKQQL |
| | | GCTCAACTTCTTGCCAACCACAGGGACCAAGCTGACCAAACAGCAGC | | ILARDILEIGAQWSILRKDIPSFE |
| | | TAATTCTGGCCCGTGACATACTGGAGATCGGGGCCCAATGGAGCATC | | RYMAQLKCYYFDYKEQLPESAYMH |
| | | CTACGCAAGGACATCCCCTCCTTCGAGCGCTACATGGCCCAGCTCAA | | QLLGLNLLFLLSQNRVAEFHTELE |
| | | ATGCTACTACTTTGATTACAAGGAGCAGCTCCCCGAGTCAGCCTATA | | RLPAKDIQTNVYIKHPVSLEQYLM |
| | | TGCACCAGCTCTTGGGCCTCAACCTCCTCTTCCTGCTGTCCCAGAAC | | EGSYNKVFLAKGNIPAESYTFFID |
| | | CGGGTGGCTGAGTTCCACACGGAGTTGGAGCGGCTGCCTGCCAAGGA | | ILLDTIRDEIAGCIEKAYEKILFT |
| | | CATACAGACCAATGTCTACATCAAGCACCCAGTGTCCTGGAGCAAAT | | EATRILFFNTPKKMTDYAKKRGWV |
| | | ACCTGATGGAGGGCAGCTACAACAAAGTGTTCCTGGCCAAGGGTCAT | | LGPNNYYSFASQQQKPEDTTIPST |
| | | CCCCGCCGAGAGCTACACCTTCTTCATTGACATCCTGCTCGACACTA | | ELAKQVIEYARQLEMIV* |
| | | TCAGGGATGAGATCGCTGGGTGCATCGAGAAGGCCTACGAGAAAATC | | |
| | | CTTTTCACTGAGGCCACCCGGATCCTCTTCTTCAACACACCCAAAAA | | |
| | | GATGACAGACTACGCCAAGAAGCGAGGGTGGGTCCTGGGCCCCAACA | | |
| | | ACTACTACAGTTTTGCCAGCCAGCAGCAGAAGCCGGAAGACACCACC | | |
| | | ATTCCCTCCACAGAACTGGCCAAACAGGTCATCGAGTATGCCCGGCA | | |
| | | GCTGGAGATGATCGTCTGA | | |
| prey2805 | 1152 | CTCTAGTCGAAGTGTAGTAGATATGGATTTAGATGATACAGATGATG | 1153 | SSRSVVDMDLDDTDDGDDNAPLFY |
| | | GTGATGACAATGCCCCTTTGTTTTACCAACCTGGGAAAAGAGGATTT | | QPGKRGFYTFRFGKNTEARLNCFR |
| | | TATACTCCAAGGCCTGGCAAGAACACAGAAGCAAGGTTGAATTGTTT | | NIGRILGLCLLQNELCFITLNRHV |
| | | CAGAAACATTGGCAGGATTCTTGGACTATGTCTGTTACAGAATGAAC | | IKVLLGRKVNWHDFAFFDPVMYES |
| | | TCTGTCCTATCACATTGAATAGACATGTAATTAAAGTATTGCTTGGT | | LRQLILASQSSDADAVFSAMDLAF |
| | | AGAAAAGTCAATTGGCATGATTTTGCTTTTTTTGATCCTGTAATGTA | | ATDLCKEECGGQVEIIPNGVNIPV |
| | | TGAGAGTTTGCGGCAAACTAATCCTCGCGTCTCAGAGTTCAGATGCT | | TPQNVYEYVRKYAEHRMLVVAEQP |
| | | 0ATGCTGTTTTCTCAGCAATGGATTTCGCATTTGCAATTGACCTGTG | | LHANRKGLLDVLPKNSLEDLTAED |
| | | TAAAGAAGAAGGTGGAGGACAGGTTGAACTCATTCCTAATGGTGTAA | | FRLLVNGCGEVNVQMLISFTSFND |
| | | ATATACCAGTCACTCCACAGAATGTATATGAGTATGTGCGG7AAAAT | | ESGENAEKLHQFKRWFWSIVEKMS |
| | | ACGCAGAACACAGAATGTTGGTAGTTGCAGAACACCCCTTACATGCA | | MTERQDLVYFWTSSPSLPASEEGF |
| | | ATGAGGAGGTCTACTAGATGTGCTTCCAAAAATTCATTAGAAGATTT | | QPMPSITIRPPDDQHLPTANTCIS |
| | | AACGGCAGAAGATTTTAGGCTTTTGGTAAATGGCTGCGGTGAAGTCA | | RLYVPLYSSKQILKQKLLLAIKTK |
| | | ATGTGCAAATGCTGATCAGTTTTACCTCTTTCAATGATGAAATCAGG | | NFGFV* |
| | | ACAAATATAAQAACCTTCTGCAGTTCAAGCGTTGGTTCTGGTCAATA | | |
| | | GTAGAGAAGATGAGCATGACAGAACGACAAGATCTTGTTTACTTTTG | | |
| | | GACATAAGCCCATCACTGCCAGCCAGTGAAAGAAGGATTCCAGCCTA | | |
| | | TGCCCTCAATCACAATAAGACCACCAGATGACCAAATCTTCCTACTA | | |
| | | CAAAAAACTTGCATTTCTCGACTTTACCTCCCACTCTATTCCTCTAA | | |
| | | ACAGATTCTCAAACAGAAATTCTTACTCGCCATTAAGACCAAGAATT | | |
| | | TTCGTTTTCTCTAC | | |
| prey100907 | 1154 | ATCTGTTTTTATCCATCTAACTTAAATCTTTTCCCAATATATAAAAA | 1155 | MCFYCSKLNLLAIYKNVNSKLYLL |
| | | ATCTAAATACTAAACTTTATTTATTAACAATCTCATCTTTTTTAATT | | RMSSFLIYIYTIVHLIYFFYTVLN |
| | | TATATTTACACAATTCTTCATCTAATTTATTTTTTCTATACACTTTT | | TQTYFAVHDIFILFSWICFPILFS |
| | | AAATACTCACACATATTTTCCTCTTCATCATATTTTTATCCTCTTCT | | LISITCWISQIIMSETEIFCIAIC |
| | | CATCCATTTCTTTTCCCATACTCTTTTCTCTCATCTCAATTACACCT | | * |
| | | TCCATCTCACAAATAATAATCTCACACACACAAATATTTTCCCACTC | | |
| | | TTCA | | |
| prey100910 | 1156 | CATATTCTATTTAATTATCTATATCCAATATTATPATATTATATATA | 1157 | DIVFNYVYAIL*YYI*YQWYCIES |
| | | ATATCACCTATTCCATTCACACTTTACAAATAACCCCTCACATTTCC | | LEISPHSRQLIFNKCVKIIIXCK* |
| | | ACACAACTTATTTTCAACAAACCTCTCAAAATAATCATTCNCCCCAA | | SSFQQMVXCNWVFRERCCNKNSPK |
| | | ATAATCATCTTTTCAAACAAAATCCTCCNCCCAAACTCCCTTTTCAC | | KKCLEVLSXSIKQW*ATEATFTPH |
| | | ACAAACACCACCCAACAAAATTCACCCAACAPAAAATCTTTCCAACT | | SKFWCYSQVFEIELIPIKALW*TF |
| | | CTTCTCCTANACCATCAAACACTCCTGACCCACTCACCCTACCTTCA | | HPKVFMX |
| | | CCCCACACTCCAACTTCTCCCCCTACTCCCACCTCCCTCACATACAC | | |
| | | CTCATTCCTATCAPACCTTTCTCGTCAAACCTTTCATCCCAAACTTT | | |
| | | TTATCCNTC | | |
| prey100911 | 1158 | CTCCANNNNNNNNNNNNNNNNNNNNNNNNNNNNNNNNNNNNNNNNNN | 1159 | LXXXXXXXXXXXXXXXXXKRKRKK |
| | | NNNNNCNAAAACCAAAACCAAAAAAACCAAACCCNTTACCATTTTTA | | RKPXRIFIFLFCXXXXFHPXPKXX |
| | | TTTTCCTTCCCCCCNANTNTAANNCATTCCACCCNNTTCCAAAANCT | | XXXXEXXKIXXXXXXXXXXXFXXP |
| | | TTNNCCNANNCTTNACAAAAANTCNADAATTTTNNTNCNNNCAANGC | | KKPXRTSXXXXDLRXLXCAXSXXC |
| | | NANNACNNTNNNTTTTNTTTNTNTNTCCNALAAAACCCNTNACAACT | | XXXKXXXVXKXNXSXCXKXIKXXF |
| | | TCCNNNTTNCNANANGATTTAAGGANNCTGGNGGGGGCTTNGTCCNT | | WVXFGXNXTXLFLXKKXXCXXFFX |
| | | CNNNGGGNGTNTANATAAANCTNGCTTNCTTTNTAAANTAAAATNCC | | FR |
| | | TCCNCNCCNNANAAAANNATTPIAANTCNATTTTTCCCTTTNATTTC | | |
| | | CCANNAACNTCACAANCTTATTTTTCANCAAAAANACNTNCCTTNCN | | |
| | | AATTTTTTANTTTCACC | | |
| prey100912 | 1160 | TTAACTTTATTTACCTCTCTCTACAATTAACATCTTTCCAAATATAT | 1161 | LTLESSLANAHLCKYIIQPSICHK |
| | | TATTCAACCAACCATTTCCCATAAACATAACCATCAACTTTCCCATT | | DKHQLSHWTSDSVQAT*LVKNKKP |
| | | CCACAACTCATACTCTTCPACCTACTTCACTTCTCAAAAACAAAAAA | | P*LLNKYILK*NMLRLINKQDIKM |
| | | CCACCATCACTTCTCAAAACAAATACATTTTAAATCPAATATCCTCA | | ETDIGTTASTLEKHKEDNGSYKXD |
| | | GGCTGATAAACAAACAAGATATTAAAATGCAGACTGACATTGGAACT | | XFIGLYXRXYWVYVLPTYGFX |
| | | ACATAGTCAACCTTGGAAAAACACAAGGAAGACAATGGCTCCTATAA | | |
| | | AANTGATTTNTTTATTGGGCTTTACCANAGANCATACTGGGTTTATG | | |
| | | TTTTACCAACTTATGGNTTTNATTT | | |
| prey95593 | 1162 | ATGTCCATAGAGAAGATCTGGGCCCGGGAGATCCTGGACTCCCCCGG | 1163 | MSIEKIIIWAREILDSRGNPTVEV |
| | | GAACCCCACAGTGGAGGTCGATCTCTATACTGCCAAAGGTCTTTTCC | | DLYTAKGLFRAAVPSGASTGIYEA |
| | | GGGCTGCAGTGCCCACTGGAGCCTCTACGGGCATCTATGAGGCCCTG | | LELRDGDKQRYLGKGVLKAVDHIN |
| | | GAGCTGAGGGATGGAGACAACAGCGTTACTTAGGCAGGTGTCCTGAA | | STIAPALTSSGLSVVEQEKLDNLM |
| | | GGCAGTGGACCACATCAACTCCACCATCGCGCCAGCCCTCATCAGCT | | LELDGTENKSKFGANAILGVSLAV |
| | | CAGGTCTCTCTGTGGTGGAGCAAGAGAAACTGGACAACCTGATGCTG | | CKAGAAERELPLYRHIAQLAGNSD |
| | | GAGTTGGATGGGACTGAGAACAAATCCAAGTTTGGGGCCAATGCCAT | | LILPVPAFNVINGGSHAGNKLAMQ |
| | | CCTGGGTGTGTCTCTGGCCGTGTGTAAGGCAGGGGCAGCTGAGCGGG | | EFMILPVGAESFRDAMRLGAEVYH |
| | | AACTGCCCCTGTATCGCCACATTGCTCAGCTGCCCGGGAACTCAGAC | | TLKGVIKDKYGKDATNVGDEGGFA |
| | | CTCATCCTGCCTGTGCCGGCCTTCAACGTGATCAATGGTGGCTCTCA | | PNILENSEALELVKEAIDKAGYTE |
| | | TGCTGGCAACAAGCTGGCCATGCAGGAGTTCATGATCCTCCCAGTGG | | KIVIGMDVAASEFYRDGKYDLDFK |
| | | GAGCTGAGAGCTTTCGGGATGCCATGCGACTAGGTGCAGAGGTCTAC | | SPTDPSRYITGDQLGALYQDFVRD |
| | | CATACACTCAAGGGAGTCATCAAGGACAAATACGGCAAGGATGCCAC | | YPVVSIEDPFDQDDWAAWSKFTAN |
| | | CAATGTGGGGGATGAAGGTGGCTTTGCCCCCAATATCCTGGAGAACA | | VGIQIVGDDLTVTNPKRIERAVEE |
| | | GTGAAGCCTTGGAGCTGGTGAAGGAAGCCATCGACAAGGCTGGCTAC | | KACNCLLLKVNQIGSVTEAIQACK |
| | | ACGGAAAAGATCGTTATTGGCATGGATGTTGCTGCCTCAGAGTTTTA | | LAQENGWGVMVSERSGETEDTFIA |
| | | TCGTGATGGCAAATATGACTTGGACTTCAAGTCTCCCACTGATCCTT | | DLVVGLCTGQIKTGAPCRSERLAK |
| | | CCCGATACATCACTGGGGACCAGCTGGGGGCACTCTACCAGGACTTT | | YNQLMRIEEELGDEARFAGHNFRN |
| | | GTCAGGGACTATCCTGTGGTCTCCATTGAGGACCCATTTGACCAGGA | | PSVL* |
| | | TGATTGGGCTGCCTGGTCCAGTTCACAGCCAATGTAGGGATCCAGAT | | |
| | | TGTGGGTGATGACCTGACAGTGACCAACCCAAAACGTATTCAGCGGG | | |
| | | CAGTGCAAGAAAAGGCCTGCAACTGTCTGCTGCTCAAGGTCAACCAG | | |
| | | ATCGGCTCGGTCACTGAAGCCATCCAAGCGTGCAAGCTGGCCCAGGA | | |
| | | GAATGGCTGGGGGGTCATGGTGAGTCATCGCTCAGGAGAGACTGACG | | |
| | | ACACATTCATTGCTGACCTGGTGGTGGGGCTGTGCACAGGCCAGATC | | |
| | | AAGACTGGTGCCCCGTGCCGTTCTGAACGTCTGGCTAAATACAACCA | | |
| | | GCTCATGAGAATTGAGGAAGAGCTGGGGGATGAGCTCGCTTTGCCGG | | |
| | | ACATAACTTCCGTAATCCCAGTGTGCTGTGA | | |
| prey7155 | 1164 | ATGGCTGCAGGCTCCCGGACGTCCCTGCTCCTGGCTTTTGCCCTGCT | 1165 | MAAGSRTSLLLAFALLCLPWLQEA |
| | | CTGCCTGCCCTGGCTTCAAGAGGCTGGTGCCGTCCAAACCGTTCCGT | | GAVQTVPLSRLFDHAMLQAHRAHQ |
| | | TATCCAGGCTTTTTGACCACGCTATGCTCCAAGCCCATCGCGCGCAC | | LAIDTYQEFEETYIPKDQKYSFLH |
| | | CAGCTGGCCATTGACACCTACCAGGAGTTTGAAGAAACCTATATCCC | | DSQTSFCFSDSIPTPSNMEETQQK |
| | | AAAGGACCAGAAGTATTCATTCCTGCATGACTCCCAGACCTCCTTCT | | SNLELLRISLLLIESWLEPVRFLR |
| | | GCTTCTCAGACTCTATTCCGACACCCTCCAACATGGAGGAAACGCAA | | SMFANNLVYDTSDSDDYHLLKDLE |
| | | CAGAAATCCAATCTAGAGCTGCTCCGCATCTCCCTGCTGCTCATCGA | | EGIQTLMGVRVAPGVANPGTPLAA |
| | | GTCGTGCCTGCAGCCCGTGCGGTTCCTCAGGAGTATGTTCGCCAACA | | |
| | | ACCTGGTGTATGACACCTCGGACAGCGATGACTATCACCTCCTAAAG | | |
| | | GACCTAGAGGAAGGCATCCAAACGCTGATGGGGGTGAGGGTGCCGCC | | |
| | | AGGGGTCGCCAATCCTGGAACCCCACTGGCTTAG | | |
| prey78905 | 1166 | GTTTCTGGGGTGGCTCTAGACCCTAAGAATAACCTGGTGATTTTCCA | 1167 | VSGVALDPKNNLVIFHRGDHVWDG |
| | | CAGAGGTGACCATGTCTGGGATGGAAACTCGTTTGACAGCAAGTTTG | | NSFDSKFVYQQIGLGPIEEDTILV |
| | | TTTACCAGCAAATAGGACTCGGACCAATTGAAGAAGACACTATTCTT | | IDPNNAAVLQSSEKNLFYLPHGLS |
| | | GTCATAGATCCAAATAATGCTGCAGTACTCCAGTCCAGTGAAAAAAA | | HDKDGNYWVTDVALHQVFKLDPNN |
| | | TCTGTTTTACTTGCCACATGGCTTGAGTATAGATAAAGATGGGAATT | | KEGPVLILGRSMQPGSDQNHFCQP |
| | | ATTGGGTCACAGACGTGGCTCTCCATCAGGTGTTCAAACTGGATCCA | | TDVAVDPGTGAIYVSDGYCNSRIV |
| | | AACAATAAAGAAGGCCCTGTATTATCCTGGGAAGGAGCATGCAACCA | | QFSPSGKFITQWGEESSGSSPLPG |
| | | GGCAGTGACCAGAATCACTTCTGTCAACCCACTGATGTGGCTGTGGA | | QFTVPHSLALVPLLGQLCVADREN |
| | | TCCAGGCACTGGAGCCATTTATGTATCAGATGGTTACTGCAACAGCA | | GRIQCFKTDTKEFVREIKHSSFGR |
| | | GGATTCTGCAGTTTTCACCAAGTGGAAAGTTCATCACACAGTGGGGA | | NVFAISYIPGLLFAVNGKPHFGDQ |
| | | GAAGAGTCTTCAGGGAGCAGTCCTCTGCCAGGCCAGTTCACTGTTCC | | EPVQGFVMNFSNGEIIDIFKPVRK |
| | | TCACAGCTTGGCTCTTGTGCCTCTTTTGGGCCAATTATGTGTGGCAG | | HFDMPHDIVASEDGTVYIGDAHTN |
| | | ACCGGGAAAATGGTCGGATCCAGTGTTTTAAAACTGACACCAAAGAA | | TVWKFTLTEKLEHRSVKKAGIEVQ |
| | | TTTGTGAGAGAGATTAAGCATTCATCATTTGGAAGAAATGTATTTGC | | EIKEAEAVVETKMENKPTSSELQK |
| | | AATTTCATATATACCAGGCTTCCTCTTTGCAGTGAATGGGAAGCCTC | | MQSKQKLIKEPGSGVPVVLITTLL |
| | | ATTTTGGGGACCAAGAACCTGTACAAGGATTTGTGATGAACTTTTCC | | VIPVVVLLAIAIFIRWKKSRAFGA |
| | | AATGGGGAAATTATAGACATCTTCAAGCCAGTGCGCAAGCACTTTGA | | DSEHKLETSSGRVLGRFRGKGSGG |
| | | TATGCCTCATGATATTGTTGCATCTGAAGATGGGACCGTGTACATTG | | LNLGNFFASRKGYSRKGFDRLSTE |
| | | GAGATGCTCATACCAACACCGTGTGGAACTTCACCTTGACTGAGAAA | | GSDQEKEDDGSESEEEYSAPLPAL |
| | | TTGGAACATCGATCAGTTAAAAAGGCTGGCATTGAGGTCCAGGAAAT | | APSSSA |
| | | CAAAGAAGCCGAGGCAGTTGTTGAAACCAAAAGGAGAACAAACCCAC | | |
| | | CTCCTCAGAATTGCAGAAGATGCAAGAGAAACAGAAGCTGATCAAAG | | |
| | | AGCCAGGCTCGGGAGTGCCTGTTGTTCTCATTACAACCCTTCTGGTT | | |
| | | ATTCCGGTGGTTGTCCTGCTGGCCATTGCCATATTTATTCGGTGGAA | | |
| | | AAAATCAAGGGCCTTTGGAGCAGATTCTGAACACAAACTCGAGACGA | | |
| | | GTTCAGGAAGAGTACTGGGAAGATTTAGAGGAAIXGGGAAGTGGAGG | | |
| | | CTTAAACCTTGGTAATTTCTTTGCAAGCCGTAAGGGCTACAGTCGAA | | |
| | | AAGGGTTTGACCGGCTTAGCACTGAGGGCAGTGACCAAGAGAAAGAG | | |
| | | GATGATGGAAGTGAATCAGAAGAGGAGTATTCAGCACCTCTGCCTGC | | |
| | | GCTCGCACCTTCCTCCTCCTGA | | |
| prey100918 | 1168 | TTCTTTTGATGATGATCATAAAATTAAATTTCAGACTTCACTAGTTC | 1169 | SFDDDHKIKFQTSLVLRNTTGCLR |
| | | TGCGTAACACGACAGGTTGCCTAAGGAACCTCACGTCCGCGGGGGAA | | NLTSAGEEARKQMRSCEGLVDSLL |
| | | GAAGCTCGGAAGCAAATGCGGTCCTGCGAGGGGCTGGTAGACTCACT | | YVIHTCVNTSDYDSKTVENCVCTL |
| | | GTTGTATGTGATCCACACGTGTGTGAACACATCCGATTACGACAGCA | | RNLSYRLELEVPQARLLGLNELDD |
| | | AGACGGTGGAGAACTGCGTGTGCACCCTGAGGAACCTGTCCTATCGG | | LLGKESPSKDSEPSCWGKKKKKKK |
| | | CAGGAGCTGGAGGTGCCCCAGGCCCGGTTACTGGGACTGAACGAATT | | RTPQEDQWDGVGPIPGLSKSPKGV |
| | | GGATGACTTACTAGGAAAAGAGTCTCCCAGCAAAGACTCTGAGCCAA | | EMLWHPSVVKPYLTLLASSSNPAT |
| | | GTTGCTGGGGGAAGAAGAAGAAAAAGAAAAAGAGGACTCCGCAAGAA | | LEGSAGSLQNLSASNWKFAAYIRG |
| | | GATCAATGGGATGGAGTTGGTCCTATCCCAGGACTGTCGAAGTCCCC | | GRPKRKGLPILVELLRMDNDKVVS |
| | | CAAAAGGGGTTGAGATGCTGTGGCACCCATCGGTGGTAAAACCATAT | | SGATALRNMALDVRNKELIGKYAM |
| | | CTGACTCTTCTAGCAGAAAGTTCCAACCCAGCCACCTTGCAAGGCTC | | RDLVNRLPGGNGPSVLSDETMAAI |
| | | TGCAGGGTCTCTCCAGAACCTCTCTGCTAGCAACTGGAAGTTTGCAG | | CCALHEVTSKNMENAKALADSGGI |
| | | CATATATCCGGGGCGGCCGTCCCAAAAGAAAAGGGCTCCCCATCCTT | | EKLVNITKGRGDRSSLKVVKAAAQ |
| | | GTGGAGCTTCTGAGAATGGATAACGATAGAGTTGTTTCTTCCGGTGC | | VLNTLWQYRDLRSIYKKDGWNQNH |
| | | AACAGCCTTGAGGAATATGGCACTAGATGTTCGCAACAAGGAGCTCA | | FITPVSTLERDRFKSHPSLSTTNQ |
| | | TAGGCAAATACGCCATGCGAGACCTGGTCAACCGGCTCCCCGGCGGC | | QMSPIIQSVGSTSSSPALLGIRDP |
| | | AATGGCCCCAGTGTCTTGTCTGATGAGACCATGGCAGCCATCTGCTG | | RSEYDRTQPPMQYYNSQGDATHKG |
| | | TGCTCTGCACGAGGTCACCAGCAAAAACATGGAGAACGCAIAI&AGC | | LYPGSSKPSPIYISSYSSPAREQN |
| | | CCTGGCCGACTCAGGAGGCATAGAGAAGCTGGTGAACATAACCAAAG | | RRLQHQQLYYSQDDSNRKNFDAYR |
| | | GCAGGGGCGACAGATCATCTCTGAAAGTGGTGAAGGCAGCAGCCCAG | | LYLQSPHSYEDPYFDDRVHFPAST |
| | | GTCTTGAATACATTATGGCAATATCGGGACCTCCGGAGCATTTATAA | | DYSTQYGLKSTTNYVDFYSTKRPS |
| | | AAAGGATGGGTGGAATCAGAACCATTTTATTACACCTGTGTCGACAT | | YRAEQYPGSPDSWVYDQDAQQRNS |
| | | TGGAGCGAGACCGATTCAAATCACATCCTTCCTTGTCTACCACCAAC | | FFLTLFRLR* |
| | | CAACAGATGTCACCCATCATTCAGTCAGTCGGCAGCACCTCTTCCTC | | |
| | | ACCAGCACTGTTAGGAATCAGAGACCCTCGCTCTGAATACGATACGA | | |
| | | CCCAGCCACCTATGCAGTATTACAATAGCCAAGGGGATGCCACACAT | | |
| | | AAAGGCCTGTACCCTGGCTCCAGCAAACCTTCACCAATTTACATCAG | | |
| | | TTCCTATTCCTCACCAGCAAGAGAACAAAATAGACGGCTACAGCATC | | |
| | | AACAGCTGTATTATAGTCAGATGACTCCAACAGAAAGAACTTTGATG | | |
| | | CATACAGATTGTATTTGCAGTCTCCTCATAGCTATGAAGATCCTTAT | | |
| | | TTTGATGACCGAGTTCACTTTCCAGCTTCTACTGATTACTCAACACA | | |
| | | GTATGGACTGAAATCGACCACAAATTATGTAGACTTTTATTCCACTA | | |
| | | AACGACCTTCTTATAGAGCAGAACAGTACCCAGGGTCCCCAGACTCA | | |
| | | TGGGTGTACGATCAAGATGCCCAACAGAGGAACTCTTTCTTTCTAAC | | |
| | | CTTGTTCAGATTGAGGTGA | | |
| prey92918 | 1170 | ATGAACTCCATGAGTCTCTCCAGGGCTGCCTGCAGCACGTCTTTTCC | 1171 | MNSMSLSRAACSTSFPSSLFGFPS |
| | | AAGTAGCCTATTTGGATTCCCATCTCAAATGTCCTGGATGCGAGCCT | | QMSWMRASAAPELGAGEVPFGEPF |
| | | CAGCGGCTCCAGAGCTCGGGGCGGGTGAGGTCCCCTTTGGGGAACCC | | PGHRGRGAAVCGQEDPRGSQERRS |
| | | TTTCCTGGCCATCGAGGTCGGGGGGCTGCCGTCTGTGGGCAGGAGGA | | LHCEKLPGCSAFSFYHGKCRLGPW |
| | | CCCGAGGGGCAGCCAGGAAAGGCGATCTCTTCACTGTGAAAAGTTGC | | GEPAGLWCCPRPPPPPECSSSLGS |
| | | CCGGGTGCAGCGCCTTTTCCTTCTACCATCGGAAATGCAGGCTGGGC | | HKQATQEEQNTASVDFPNFPGKA |
| | | CCTTGGGGTGACCCTGCGGGGCTCTGGTGCTGTCCCCGACCCCCACC | | |
| | | ACCACCAGAATGCAGTTCCAGCTTAGGAAGCCACAAACAAGCCACCC | | |
| | | AGGAGGAACAAAACACCGCCAGCGTGGATTTTCCATTTCCCTGGAAA | | |
| | | GTAA | | |
| prey49397 | 1172 | CCAGACGGGACCCAGCGTCACAGTGACCTGTACAGAGGGCAAGTGGA | 1173 | QTGPSVTVTCTEGKWNKQVACEPV |
| | | ATAAGCAGGTGGCCTGTGAGCCAGTCGACTGCAGCATCCCAGATCAC | | DCSIPDHHQVYAASFSCPEGTTFG |
| | | CATCAAGTCTATGCTGCCTCCTTCTCCTGCCCTGAGGGCACCACCTT | | SQCSFQCRHPAQLKGNNSLLTCME |
| | | TGGCAGTCAATGTTCCTTCCAGTGCCGTCACCCTGCACAATTGAAAG | | DGLWSFPEALCELMCLAPPPVPNA |
| | | GCAACAACAGCCTCCTGACCTGCATGGAGGATGGGCTGTGGTCCTTC | | DLQTARCRENKHKVCSFCKYKCKP |
| | | CCAGAGGCCCTGTGTGAGCTCATGTGCCTCGCTCCACCCCCTGTGCC | | GYHVPGSSRKSKKRAFKTQCTQDG |
| | | CAATGCAGACCTCCAGACCGCCCGGTGCCGAGAGAATAAGCACAAGG | | SWQEGACVPVTCDPPPPKFHGLYQ |
| | | TGGGCTCCTTCTGCAAATACAAATGCAAGCCTGGATACCATGTGCCT | | CTNGFQFNSECRIKCEDSDASQGL |
| | | GGATCCTCTCCCAAGTCAAAGAAACGGGCCTTCAACACTCACATGTA | | GSNVIHCRKDGTWNGSFHVCQEMQ |
| | | CCCAGGATGGCAGCTGGCACGAGGGACCTTGTGTTCCTGTGACCTGT | | GQCSVPNELNSNLKLQCPDGYAIG |
| | | GACCCACCTCCACCAAATTCCATGGGCTCTACCAGTGTACTAATGGC | | SECATSCLDHNSESIILPMNVTVR |
| | | TTCCAGTTCAACAGTGAGTGTAGGATCAAGTGTGAAGACAGTGATGC | | DIPHWLNPTRVERVVCTAGLKWYP |
| | | CTCCCAGGGACTTCGGAGCAATCTCATTCATTGCCGGAAAAGATGGC | | HPALIHCVKGCEPFMGDNYCDAIN |
| | | ACCTGGAACGGCTCCTTCCATGTCTGCCACGACATGCAAGCCCAGTG | | NRAFCNYDGGDCCTSTVKTKKVTP |
| | | CTCGGTTCCAAACGAGCTCAACAGCAACCTCAAACTGCAGTGCCCTG | | FPMSCDLQGDCACRDPQAQEHSRK |
| | | ATGCCTATGCCATAGGGTCGGAGTGTGCCACCTCGTGCCTGGACCAC | | DLRGYSHG* |
| | | AACACCGAGTCCATCATCCTGCCAATCAACGTGACCGTGCGTGACAT | | |
| | | CCCCCACTGGCTGAACCCCACACGGGTAGAGAGAGTTGTCTGCACTC | | |
| | | CTGGTCTCAAGTGGTATCCTCACCCTGCTCTGATTCACTGTGTCAAA | | |
| | | GGCTGTGAGCCCTTCATGCGAGACAATTATTGTGATGCCATCAACAA | | |
| | | CCGAGCCTTTTGCAACTATGACGGTGGGGATTCCTCCACCTCCACAG | | |
| | | TGAAGACCAAAAAGGTCACCCCATTCCCTATGTCCTGTGACCTACAA | | |
| | | GGTCACTCTGCTTGTCGGGACCCCCAGGCCCAAGAACACAGCCGGAA | | |
| | | AGACCTCCGGGGATACACCCATGGCTAA | | |

1. A complex between two polypeptides of adipocytes as defined in columns 1 and 4 in Table 2.

2. A complex between two polynucleotides of adipocytes, said polynucleotides encoding two polypeptides as defined in columns 1 and 4 in Table 2.

3. A recombinant host cell expressing a polynucleotide encoding an adipocyte polypeptide as defined in columns 1 and 4 in Table 2.

4. A method for selecting a modulating compound that inhibits or activates interactions between two polypeptides of adipocytes comprising:
cultivating a recombinant host cell containing a reporter gene the expression of which is toxic for said recombinant host cell, on a selective medium comprising a modulating compound, wherein said recombinant host cell is transformed with two vectors:
wherein said first vector comprises a polynucleotide encoding a first hybrid polypeptide and a DNA binding domain;
wherein said second vector comprises a polynucleotide encoding a second hybrid polypeptide and an activating domain that activates said reporter gene when the first and second hybrid polypeptides interact; and
selecting said modulating compound which inhibits the growth of said recombinant host cell.

5. A modulating compound obtained from the method of claim 4.

6. A SID polypeptide comprising SEQ ID NOS: 28, 30 or 32, or odd sequences starting from SEQ ID NOS: 49 to 1173, inclusive, in column 4 of Table 3, or a fragment or a variant thereof.

7. A variant of the SID polypeptide of claim 6.

8. A fragment of the SID polypeptide of claim 6.

9. A SID polynucleotide comprising SEQ ID NOS: 27, 29 or 31, or even sequences starting from SEQ ID NOS: 48 to 1172, inclusive, in column 2 of Table 3, or a fragment or a variant thereof.

10. A fragment of the SID polynucleotide of claim 9.

11. A variant of the SID polynucleotide of claim 9.

12. A vector comprising the SID polynucleotide of claim 9.

13. A vector comprising the fragment of the SID polynucleotide of claim 10.

14. A recombinant host cell comprising the vector of claim 12.

15. A pharmaceutical composition comprising a modulating compound that inhibits or activates interactions between polypeptides of adipocytes, and a pharmaceutically acceptable carrier.

16. A pharmaceutical composition comprising a SID polypeptide designated by SEQ ID NOS: 28, 30 or 32, or odd sequences starting from SEQ ID NOS: 49 to 1173, inclusive, in column 4 of Table 3, and a pharmaceutically acceptable carrier.

17. A pharmaceutical composition comprising the recombinant host cell of claim 14 and a pharmaceutically acceptable carrier.

18. A protein chip comprising a polypeptide of adipocytes as defined in columns 1 and 2 of Table 1.

19. A record comprising all or part of the data set forth in Tables 1 and 2.
